(19) United States
(12) Patent Application Publication
(10) Pub. No.: US 2021/0317092 A1
(43) Pub. Date: Oct. 14, 2021

(54) TETRAZOLE CONTAINING COMPOUNDS

(71) Applicant: BAYER PHARMA AKTIENGESELLSCHAFT, Berlin (DE)

(72) Inventors: Stefan BÄEURLE, Berlin (DE); Adam James DAVENPORT, Abingdon Oxfordshire (GB); Christopher STIMSON, Abingdon Oxfordshire (GB); James Lindsay CARR, Abingdon Oxfordshire (GB); Christian Abingdon BUBERT, Abingdon Oxfordshire (GB); Frédéric Jacques MARLIN, Abingdon Oxfordshire (GB); Jens NAGEL, Daxweiler (DE); Nicole SCHMIDT, San Francisco, CA (US); Andrea ROTGERI, Berlin (DE); Horst IRLBACHER, Berlin (DE)

(21) Appl. No.: 16/472,281

(22) PCT Filed: Dec. 18, 2017
(86) PCT No.: PCT/EP2017/083284

§ 371 Date: Jun. 21, 2019

(30) Foreign Application Priority Data
Dec. 23, 2016 (EP) 16206746.6

(51) Int. Cl. C07D 257/04; A61P 19/02; A61P 29/00; C07D 401/12; C07D 401/10; C07D 401/14; C07D 403/10; C07D 491/048; C07D 409/10

(52) U.S. Cl. CPC C07D 257/04; A61P 19/02; A61P 29/00; C07D 401/12; C07D 409/10; C07D 401/14; C07D 403/10; C07D 491/048; C07D 401/10

(57) ABSTRACT

The present invention relates to tetrazole containing compounds of general formula (I) as described and defined herein, to pharmaceutical compositions and combinations comprising said compounds and to the use of said compounds for manufacturing a pharmaceutical composition for the treatment or prophylaxis of a disease syndrome, condition, or symptoms, in particular related to chronic pain and inflammation, as a sole agent or in combination with other active ingredients.

The present invention relates to tetrazole containing phenyl or pyridinyl compounds of general formula (I) as described and defined herein, to pharmaceutical compositions and combinations comprising said compounds and to the use of said compounds for manufacturing a pharmaceutical composition for the treatment or prophylaxis of a disease, in particular of Bradykinin B1 receptor associated disorders which are related to inflammation or at least partially driven by neurogenic events like diseases related to chronic pain or frequent pain conditions like but not restricted to osteoarthritis, rheumatoid arthritis, gout, inflammatory bowel disease, and endometriosis and diseases related to Bradykinin B1 receptor activation and/or up-regulation in affected tissue like but not restricted to asthma, fibrosis in various tissues or diabetes as a sole agent or in combination with other active ingredients.

### BACKGROUND OF THE INVENTION

The present invention relates to chemical compounds that antagonize the effects of human Bradykinin B1 receptor (Gene Name BDKRB1, Gene ID 623).

The Bradykinin B1 receptor is a membrane-bound G-protein coupled receptor, which is linked to a second messenger system that triggers increase of intracellular calcium concentrations.

The main signalling pathway is linked to Gq protein and phospholipase C (Leeb-Lundberg, L. M. et al. (2005), Pharmacol Rev 57(1): 27-77). Activation of Bradykinin B1 receptor has been shown to be pro-algesic, pro-fibrotic, and proinflammatory while Bradykinin B1 receptor antagonists had clear anti-inflammatory and analgesic effects in various animal models (Gougat, J. B. et al. (2004), J Pharmacol Exp Ther 309(2): 661-669; Dias, J. P. et al. (2007), Br J Pharmacol 152(2): 280-287; Schuelert, N. et al. (2015), Eur J Pain 19(1): 132-142). As consequence of Bradykinin B1 receptor activity increased gene expression and protein levels of proinflammatory cytokines like e.g. Il-6 and Il-8 that attract and activate inflammatory leucocytes, increase of PGE2 (Prostaglandin 2) levels and therefore activation of the inflammation related prostaglandin pathway, phosphorylation and upregulation of TRPV1 (Transient Receptor Potential Vanilloid 1) receptors which are important mediators of pain transduction and induction of neurogenic inflammation (neuropeptide release in inflamed tissue) were observed (Phagoo, S. B. et al. (1999). Mol Pharmacol 56(2): 325-333; Westermann, D. et al. (2009), Diabetes 58(6): 1373-1381; Walsh, D. A. et al. (2006), Curr Drug Targets 7(8): 1031-1042; Farkas S. et al. (2011), Drugs of the Future 36(4): 301-319). Bradykinin B1 receptor agonists are endogenously produced by the activated kallikrein-kinin system. This system consists of circulating kininogens, the ubiquitous expressed proteolytic enzymes kallikreins which are activated by tissue damage, and kinins which are formed by activated kallikreins out of kininogens (Review Fincham, C. I. et al. (2009), Expert Opin Ther Pat 19(7): 919-941). These kinins (e.g. bradykinin, kalidin, des-Arg9-bradykinin, des-Arg10-kalidin) are proinflammatory peptides that mediate vascular and pain responses to tissue injury, with functions in cardiovascular homeostasis, contraction or relaxation of smooth muscle, inflammation and nociception. They exert most of their effects by interacting with two classes of G-protein-coupled receptors called Bradykinin receptor 1 and 2. The classification of the kinin receptors was originally achieved by means of pharmacological studies originally carried out at the end of the 1970s. During the 1990s, the existence of Bradykinin B1 receptor and B2 receptors was further confirmed through cloning and genetic deletion studies (Menke, J. G. et al. (1994), J Biol Chem 269(34): 21583-21586). The past 30 years of research on the kinin system has indicated that both Bradykinin B1 receptor and B2 receptor are involved in pain and inflammation (Leeb-Lundberg, L. M. et al. (2005), Pharmacol Rev 57(1): 27-77; Marceau, F. (2005), Trends Pharmacol Sci 26(3): 116-118; Marceau, F. (2004), Nat Rev Drug Discov 3(10): 845-852; Chen, J. J. et al. (2007), Expert Opin Ther Targets 11(1): 21-35).

It has been demonstrated that the B2 receptor is widely expressed in a constitutive manner throughout most mammalian tissues. In contrast, the Bradykinin B1 receptor is not constitutively expressed to a great extent under normal conditions, but is up-regulated under various inflammatory conditions such as asthma, arthritis and osteoarthritis, sepsis and type-1 diabetes, as well as by some neuropathological diseases such as epilepsy, stroke and multiple sclerosis. Bradykinin B1 receptor up-regulation can be induced for example by Il-1beta (Phagoo, S. B. et al. (1999), Mol Pharmacol 56(2): 325-333) and Bradykinin B2 receptor activation (NF-kB activation leading to IL1 b expression in fibroblasts) (Leeb-Lundberg, L. M. et al. (2005), Pharmacol Rev 57(1): 27-77).

Once upregulated, the Bradykinin B1 receptor is expressed on neurons, macrophages, neutrophils, fibroblasts, smooth muscle cells and the vascular endothelium (Fincham, C. I. et al. (2009), Expert Opin Ther Pat 19(7): 919-941). Recent findings suggest that the Bradykinin B1 receptor expressed in the peripheral and in the central nervous system is involved in processing of inflammatory pain (Schuelert, N. et al. (2015). Eur J Pain 19(1): 132-142).

In contrast to Bradykinin B2 receptor and many other GPCRs (G protein-coupled receptors), the Bradykinin B1 receptor does not show agonist induced internalization or relevant desensitization (Prado, G. N. et al. (2002), J Cell Physiol 193(3): 275-286; Eisenbarth, H. et al. (2004), Pain 110(1-2): 197-204). Activation of Bradykinin B1 receptor triggers auto-induction of the receptor. This might lead to an augmentation of the inflammatory or pain-inducing processes.

Therefore, Bradykinin B1 receptor has been suggested to have a pivotal role including but not limited to several chronic diseases involving diabetes, fibrosis, inflammation, neuroinflammation, neurodegeneration, inflammatory pain, and neuropathic pain (Campos, M. M. et al. (2006), Trends Pharmacol Sci 27(12): 646-651; Wang, P. H. et al. (2009), Int Immunopharmacol 9(6): 653-657; Passos, G. F. et al. (2013), Am J Pathol 182(5): 1740-1749; Gobeil, F. et al. (2014), Peptides 52: 82-89; Huart, A. (2015), Front Pharmacol 6: 8). The contribution of Bradykinin B1 receptor activation in inflammation and pain processes is supported by the demonstration that Bradykinin B1 receptor knockout mice have a largely decreased response to nociceptive and proinflammatory stimuli (Ferreira, J. et al. (2001), Neuropharmacology 41(8): 1006-1012; Ferreira, J. et al. (2005), J Neurosci 25(9): 2405-2412.). The therapeutic impact of Bradykinin B1 receptor blockage for inflammation related diseases is supported further by the pharmacological properties of Bradykinin B1 receptor antagonists shown in many inflammatory and neuropathic pain models (Gougat, J. B. et al. (2004), J Pharmacol Exp Ther 309(2): 661-669; Fox, A. et al. (2005), Br J Pharmacol 144(7): 889-899).

The fact that Bradykinin B1 receptor expression is induced under disease conditions clearly raises the possibility that therapeutic use of Bradykinin B1 receptor antagonists should be devoid of undesired adverse effects. This property supports the suitability of Bradykinin B1 receptor antagonists for treatment of benign diseases like endometriosis due the expected positive risk benefit ratio. The patient populations for nociceptive pain and neuropathic pain are large, and are driven by separate disease trends that necessitate pain relief. Chronic pain of moderate to severe intensity occurs in 19% of adult Europeans, seriously affecting the quality of their social and working lives (Breivik et al., Eur J Pain. 2006 May; 10(4):287-333.). Unfortunately, current treatments for pain are only partially effective, and many cause life-style altering, debilitating, and/or dangerous side effects. For example, non-steroidal anti-inflammatory drugs (NSAIDS) such as aspirin, ibuprofen, and indomethacin are moderately effective against inflammatory pain but they are also renally toxic, and high doses tend to cause gastrointestinal irritation, ulceration, bleeding, confusion and increased cardiovascular risk. Notably, Vioxx was withdrawn from the market in 2004 due to a risk of myocardial infarction and stroke. Patients treated with opioids frequently experience confusion and constipation, and long-term opioid use is associated with tolerance and addiction. Local anaesthetics such as lidocaine and mexiletine simultaneously inhibit pain and cause loss of normal sensation. In addition, when used systemically, local anaesthetics are associated with adverse cardiovascular effects. Thus, there is currently an unmet need in the treatment of chronic pain in general.

Especially in gynaecological therapy field, endometriosis is the diseases associated with chronic pelvic pain severely affecting quality of life of the patients. Globally, approximately 11% of women aged 15-49 years are affected by endometriosis and additional 6% of women suffer from symptoms suggestive for endometriosis. Main symptoms of endometriosis are chronic or frequent pelvic pain, dyspareunia, dyschezia, dysuria and sub- or infertility. These symptoms severely impair quality of life of patients. Diagnosis of the disease involves a complete medical history, a physical examination and a laparoscopy. As an ultimate confirmation of endometriosis can only be made invasively and symptoms are often unspecific, the mean time from initial symptoms to diagnosis of endometriosis is about 7-10 years. Therefore, endometriosis is under-diagnosed and the number of affected women might be much higher than anticipated. Recently published EndoCost study demonstrated that cost of productivity loss of €6,298 per woman were double the healthcare cost of €3,113 per women, driven mainly by surgery and monitoring visit (Gao, X. et al. (2006), Fertil Steril 86(6): 1561-1572; Simoens 5, et al. Hum Reprod (2012), 27(5):1292-9; De Graaff A, et al. (2013), Hum Reprod; 28(10): 2677-85).

Endometriosis is characterized by growth of endometrial tissue outside of the uterine cavity forming benign tumours (lesions) in the affected part of the body. Depending on lesion location and innervation severity of pain symptoms is observed. Up-regulation of various inflammation markers observed in the affected tissue and in the peritoneal tissue underline the inflammatory character of the disease (Stratton, P. et al. (2011), Hum Reprod Update 17(3): 327-346; Gao, X. et al. (2006), Fertil Steril 86(6): 1561-1572; Laux-Biehlmann et al. (2015), Trends Pharmacol Sci 36(5): 270-276).

The Bradykinin B1 receptor was identified in endometriosis lesion by immune-histological-chemical (IHC) staining (Yoshino et al. Journal of Reproductive Immunology 112 (2015) 121-140; www.proteinatlas.org) and analysis of m-RNA expression of Bradykinin B1 receptor in affected tissue shows a positive correlation to pain severity reported by endometriosis patients. Data describing a role of Bradykinin B1 receptors in affecting the outcome of an endometriosis mouse model (Jingwei, C. et al. (2015), J Tradit Chin Med 35(2): 184-191) further support the concept to treat endometriosis with Bradykinin B1 receptor antagonists.

Suspected endometriosis is initially treated with non-steroidal anti-inflammatory drugs (NSAID) or combined oral contraceptives (COC) which are used off label. This procedure delays endometriosis diagnosis. Laparoscopy is the gold standard for endometriosis diagnosis which is performed when the initial treatment options fail. During laparoscopy, endometriotic lesions are ablated. However, this procedure is accompanied by a high recurrence rate. Approximately, 70% of treated patients have persistent symptoms that are not managed. Currently, there is no long-term medication available in COC/P (Combined Oral Contraceptives/Progestin) non-responder endometriosis patients in which COCs and progestins failed. Treatment with Gonadotropin Releasing Hormone (GnRH) agonists, which are used as second line therapy (without proof of being superior versus first line) are only approved for short-term treatment (6 months). After GnRH agonist application, systemic estradiol levels are suppressed up to 90% leading to chemical castration with menopausal side effects like bone mass loss and hot flushes. Therefore, new and long-term treatment options with reduced side-effects and high efficacy for patients with COC/P non-responder endometriosis are urgently needed.

On this background the Bradykinin B1 receptor antagonists are of value for treatment of disorders which are related to inflammation or at least partially driven by neurogenic events like diseases related to chronic pain or frequent pain conditions like but not restricted to osteoarthritis (Kaufman, G. N. et al. (2011), Arthritis Res Ther 13(3): R⁷⁶), rheumatoid arthritis (Cassim, B. et al. (2009), Rheumatology 48(5): 490-496), gout (Silva, C. R. et al. (2016), Ann Rheum Dis 75(1): 260-268), burn injuries and sunburn (Eisenbarth, H. et al. (2004), Pain 110(1-2): 197-204), inflammatory bowel disease, endometriosis (Yoshino et al. Journal of Reproductive Immunology 112 (2015) 121-140; Laux-Biehlmann et al. (2015), Trends Pharmacol Sci 36(5): 270-276; Jingwei, C. et al. (2015), J Tradit Chin Med 35(2): 184-191), pre-eclampsia (Moyes, A. J. et al. (2014), Hypertens Pregnancy 33(2): 177-190), diabetic neuropathy (Dias, J. P. et al. (2007), Br J Pharmacol 152(2): 280-287) including neuropathy related to diabetes type 1 and diabetes type 2, cardiac inflammation (Westermann, D. et al. (2009), Diabetes 58(6): 1373-1381), renal inflammation (Bascands, J. et al. (2009), Biochem Biophys Res Commun 386(2): 407-412), pancreatitis and diseases related to Bradykinin B1 receptor activation and/or up-regulation in affected tissue like but not restricted to asthma and cough (Bertram, C. M. et al. (2009), J Leukoc Biol 85(3): 544-552), atherosclerosis, diabetes (Dias, J. P. et al. (2012), J Cardiovasc Pharmacol 60(1): 61-69), adipositas including metabolic syndrome (Dias, J. P. et al. (2012), Diabetes Obes Metab 14(3): 244-253), diseases related to muscle atrophy including cachexia (Parreiras, E. S. L. T. et al. (2014), Clin Sci 127(3): 185-194) not limited to cancer cachexia, neuropathic pain (Luiz, A. P. et al. (2015), Neuroscience 300: 189-200), pruritus or itch (Hosogi, M. et al. (2006), Pain 126(1-3): 16-23), cancer (da Costa, P. L. et al. (2014), Cancer Lett 345(1): 27-38), neurodegenerative diseases such as amyotrophic lateral sclerosis (ALS) or Alzheimer's disease (Lacoste, et al. (2013) J Neuroinflammation 10: 57), fibrosis in cardiacs (Westermann, D. et al. (2009), Diabetes 58(6): 1373-1381), fibrosis in renal (Huart, A. et al. (2015), Front Pharmacol 6: 8) and fibrosis in lung tissues, overactive urinary bladder syndrome and cystitis (Forner, S. et al. (2012), Br J Pharmacol 167(8): 1737-1752 and Belichard, P. etat (1999), Br J Pharmacol 128(1):213-219), impaired or painful wound heating (Schremmer-Danninger, E. et al. (2004), Biol Chem 385(11): 1069-1076) and sepsis (Murugesan, P et al. (2016), J Infect Dis 213(4): 532-540).

Several new Bradykinin B1 receptor antagonists are known from prior art (Expert Opinion on Therapeutic Patents (2012), 22:12, 1443-1452). Various approaches for finding new Bradykinin B1 receptor antagonists are described, in particular peptidic structures and small molecules. Especially, arylsulfonamides and so-called cyclopropyl-carboxamides as the two main types of small molecules were investigated during the last decade.

WO2003/065789 (Merck) disclose bradykinin B1 receptor antagonists or inverse agonists of the following general formula

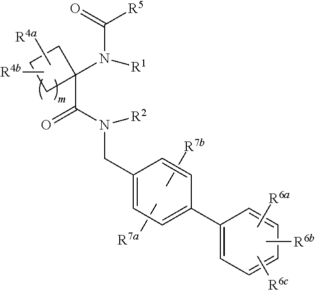

which are useful in the treatment or prevention of symptoms such as pain and inflammation associated with the bradykinin B1 pathway.

Merck was developing the bradykinin B1 receptor antagonist MK-0686 (structure shown below)

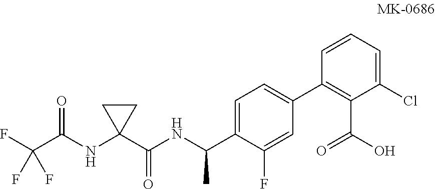 [SMILES: C[C@@H](NC(=O)C1(NC(=O)C(F)(F)F)CC1)c1ccc(-c2cccc(Cl)c2C(=O)O)cc1F]

for the potential treatment of pain and inflammation. Several phase II trials in subjects with osteoarthritis and with post-herpetic neuralgia were initiated. Merck accounted that the compound has a suboptimal pharmacokinetic profile due to metabolic tability.

Jerini AG, now Shire Group, investigated active Bradykinin B1 receptor antagonists, for example (see WO2009/036996)

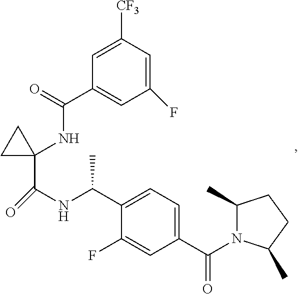 [SMILES: C[C@@H]1CC[C@H](C)N1C(=O)c1ccc([C@@H](C)NC(=O)C2(NC(=O)c3cc(F)cc(C(F)(F)F)c3)CC2)c(F)c1]

which was reported to have in addition to its activity and acceptable penetration profile reasonable aqueous solubility and pharmacokinetic profile in rat, whereas its human metabolic stability was still poor (Schaudt M, Locardi E, Zischinsky G, et al., Bioorg Med Chem Lett 2010; 20:1225-8). Jerini exchanged the cyclopropyl-carboxamide moiety to a semicarbazide or to a five-membered diamino-heterocyclic ring or even to hydroxyureas without any explanation.

Starting with arylsulfonamide compounds as Bradykinin B1 receptor antagonists, Boehringer Ingelheim reported about several cyclopropyl-carboxamides out of their further development compounds like of the following structure

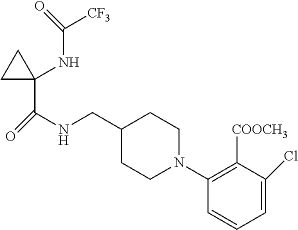 [SMILES: COC(=O)c1c(Cl)cccc1N1CCC(CNC(=O)C2(NC(=O)C(F)(F)F)CC2)CC1]

or related to that emerged with the highest binding affinity measured on human B1R-expressing CHO cells (Expert Opinion on Therapeutic Patents (2012), 22:12, 1443-1452).

In WO2012059776 Gedeon Richter reported about cyclopropyl-carboxamides of the following formula

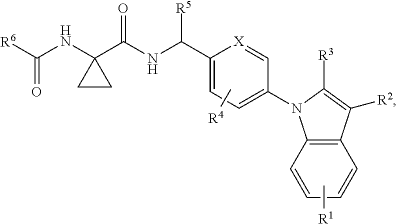

wherein R³ is selected from (1) —COOR; (2) —CN; (3) —CONR^{a}R^{b};

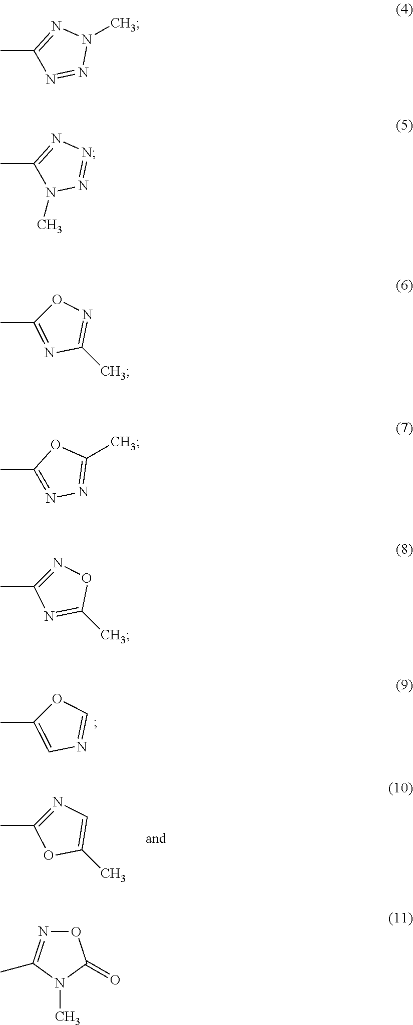

A majority of the compounds have a Kᵢ value below 20 nM on human recombinant Bradykinin receptors (expressed in CHO cells). Several indolyl compounds substituted with a tetrazol moiety are disclosed and represented by the following compound:

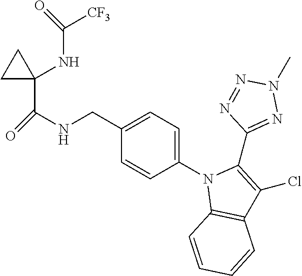 [SMILES: Cn1nnc(-c2c(Cl)c3ccccc3n2-c2ccc(CNC(=O)C3(NC(=O)C(F)(F)F)CC3)cc2)n1]

WO2005085227 (Smith Kline Beecham) discloses inhibitors of protein kinase B (PKB/Akt, PKB or Akt) of the formula

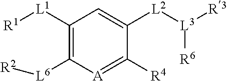

wherein
A is selected from: nitrogen, —C-halogen and —CH;
R¹ is selected from the group consisting of aryl, substituted aryl, cycloalkyl, substituted cycloalkyl, heterocycle and substituted heterocycle;
R² is selected from alkyl, substituted alkyl, cycloalkyl, substituted cycloalkyl, heterocycle, substituted heterocycle, and a cyclic or polycyclic aromatic ring,
L² is selected from the group consisting of bond, —O—, heterocycle, —N(R⁵)—, —N(R⁵)C(O)—, —S—, —S(O)—, —S(O₂)—, and —C(O)N(R⁵)—; and
L¹ as well as L⁶ can be a bond, —O—, —N(R⁵)—, —S—, —S(O), —S(O₂)—, alkyl, and —N(R⁵)C(O)—. Neither
L¹ nor L⁶ can be a heteroaryl or heterocyclic group. R⁴ is defined as hydrogen or halogen. The compounds are suitable for the treatment of cancer and arthritis. Tetrazole-substituted phenyl or pyridinyl compounds are not specifically disclosed.

In WO2012112567 (Georgetown University) small molecule inhibitors of ATP/GTP binding protein like 2 (AGBL2) of the formula

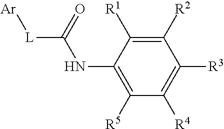

are disclosed wherein R² as well as R⁴ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, substituted or unsubstituted carbonyl, or substituted or unsubstituted carboxyl.

The compounds can be used in methods for treating or preventing cancer and neurologic disorders were described. A tetrazole moiety as substituent at the benzene core structure is not specifically disclosed.

WO2009005638 (Merck) discloses a novel class of pyridinyl and pyrimidinyl derivatives of the formula

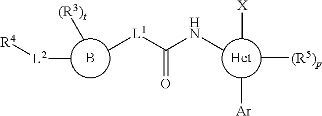

wherein the substituent Ar is aryl or heteroaryl, optionally substituted with halo, methyl, methoxy, halomethyl, amino, hydroxyl, C(O)OCH₃ or C(O)NHCH₃, X can be OH, SH or NH₂ and R⁵ is selected from H, OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-S O₂NH—, C₁-C₇ alkylsulfonyl, C₁-C₇ alkylamino or di(C₁-C₇)alkylamino. Neither X nor R⁵ can be a heteroaryl or heterocyclic group. Tetrazolyl is not specifically disclosed as substituent Ar. The compounds can be used to treat cancer.

WO2012103583 (Bionomics) discloses 1,2-cyclopropyl-carboxamide compounds of formula (I)

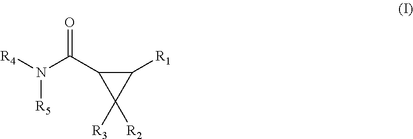

wherein R⁴ is selected from optionally substituted heteroaryl, optionally substituted heterocyclyl, or optionally substituted aryl, and R⁵ is selected from hydrogen or optionally substituted alkyl. Such compounds are useful in the positive modulation of the alpha 7 nicotinic acetylcholine receptor (□7nAChR). The disclosure of WO2012103583 also relates to the use of these compounds in the treatment or prevention of a broad range of diseases in which the positive modulation of □7nAChR is advantageous, including neurodegenerative and neuropsychiatric diseases and inflammatory diseases.

WO2007087066 (Vertex) discloses novel compounds and pharmaceutically acceptable compositions thereof, which are useful as modulators of ATP-Binding Cassette (“ABC”) transporters or fragments thereof, including Cystic Fibrosis Transmembrane Conductance Regulator (“CFTR”), having a benzamide core structure (I)

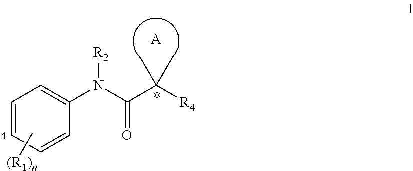

wherein ring A is an optionally substituted cycloaliphatic or an optionally substituted heterocycloaliphatic where the atoms of ring A adjacent to C* are carbon atoms. R₄ is an optionally substituted aryl or an optionally substituted heteroaryl. R¹ is independently an optionally substituted C₁-C₆ aliphatic, an optionally substituted aryl, an optionally substituted heteroaryl, an optionally substituted C₃-C₁₀ membered cycloaliphatic or an optionally substituted 4 to 10 membered heterocycloaliphatic, carboxy, amido, amino, halo, or hydroxy, provided that at least one R¹ is an optionally substituted aryl or an optionally substituted heteroaryl and said R¹ is attached to the 3- or 4-position of the phenyl ring. Compounds in which the phenyl ring of the benzamide core structure is substituted with tetrazolyl are not disclosed.

So, the state of the art described above does not describe the specific compounds of general formula (I) of the present invention containing a tetrazol moiety as defined herein or an isomer, enantiomer, diastereomer, racemate, hydrate, solvate, or a salt thereof, or a mixture of same, as described and defined herein, and as hereinafter referred to as “compounds of the present invention”, or their pharmacological activity.

### SUMMARY OF THE INVENTION

The present invention covers tetrazole containing compounds of general formula (I):

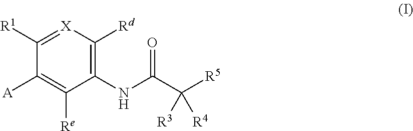

in which
A represents tetrazolyl which is attached to the rest of the molecule by the carbon atom;
R¹ represents
   phenyl,
   C₃-C₇-cycloalkyl,
   5- to 7-membered lactam,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 8- to 10-membered heteroaryl containing 1, 2 or 3 heteroatoms or heteroatom-containing groups independently selected from NH, N, O, S, SO and SO₂,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, benzyl, NHR², N(R²)₂, NH(C₃-C₅-cycloalkyl), halogen, CN, NHSO₂R², SO₂R² or 4- to 7-membered heterocycloalkyl containing 1 or 2 heteroatoms or heteroatom-containing groups selected from NH, —NR², N, O, S, SO and SO₂, and
   wherein independently, if R¹ represents 5- to 7-membered lactam, 5-membered heteroaryl or bicyclic 8- to 10-membered heteroaryl, each ring nitrogen atom, if present, of said R¹ is optionally substituted with a substituent R^{1b} wherein R^{1b}represents C₁-C₅-alkyl, C₃-C₅-cycloalkyl, —(C₁-C₃-alkyl)-(C₃-C₅-cycloalkyl), benzyl, SO₂R² or 4- to 7-membered heterocycloalkyl containing 1 or 2 heteroatoms or heteroatom-containing groups selected from NH, —NR², N, O, S, SO and SO₂, and
      if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F, and
      if R^{1a} and/or R^{1b} represent 4- to 7-membered heterocycloalkyl, each carbon atom of said 4- to 7-membered heterocycloalkyl is optionally substituted with one or more substituents independently selected from the group consisting of OH, OR² and F;
R² represents C₁-C₅-alkyl optionally substituted with 1 to 5 fluorine atoms;
X represents CR^{c} or N;
R^{c} represents H, halogen, CN, C₁-C₅-alkyl, or —OC₁-C₅-alkyl wherein said C₁-C₅-alkyl and —OC₁-C₅-alkyl are optionally substituted with 1 to 5 fluorine atoms;
R^{d} represents H, halogen, CN, OH, C₁-C₅-alkyl, or —OC₁-C₅-alkyl wherein said C₁-C₅-alkyl and —OC₁-C₅-alkyl are optionally substituted with 1 to 5 fluorine atoms;
R^{e} represents H, halogen or OH;
R³ and R⁴ are the same or different and represent C₁-C₃-alkyl, or
R³ and R⁴ may be conjoined together with the carbon atom to which R³ and R⁴ are attached to form a C₃-C₅-cycloalkyl, which is optionally substituted with one or two C₁-C₃-alkyls wherein said C₁-C₃-alkyls are optionally substituted with 1 to 5 fluorine atoms;
R⁵ represents
   C₁-C₅-alkyl,
   (CH₂)ₚ-phenyl,
   —(CH₂)ₚ-(C₅-C₇-cycloalkyl),
   5- or 6-membered heteroaryl, wherein said 5-membered heteroaryl contains 1, 2 or 3 heteroatoms or heteroatom-containing-groups independently selected from the group consisting of S, N, NH, and O, and wherein said 6-membered heteroaryl contains 1 or 2 nitrogen atoms, or
   bicyclic 8- to 10-membered heteroaryl containing 1, 2 or 3 heteroatoms or heteroatom containing groups independently selected from NH, N, O, S, SO and SO₂,
   wherein said R⁵ is optionally substituted at one or more carbon atoms with 1 to 3 substituents which are the same or different and selected from the group consisting of C₁-C₅-alkyl, —OC₁-C₅-alkyl, halogen, OH and CN, wherein said C₁-C₅-alkyl and —OC₁-C₅-alkyl independently are optionally substituted with (a) substituent(s) independently selected from the group consisting of OH, OR², and 1 to 5 fluorine atoms, and
   wherein independently, if R⁵ represents 5-membered heteroaryl or bicyclic 8- to 10-membered heteroaryl, each ring nitrogen atom, if present, of said R⁵ is optionally substituted with C₁-C₅-alkyl, which is optionally substituted with OH, OR² or 1 to 5 fluorine atoms; and
p is 0 or 1;
   or an isomer, enantiomer, diastereomer, racemate, hydrate, solvate, or a salt thereof, or a mixture of the same.

The present invention further relates to pharmaceutical compositions and combinations comprising said compounds, to use of said compounds for manufacturing a medicament for the treatment or prophylaxis of diseases or disorders and for the treatment of pains, which are associated with such diseases.

It has now been found, and this constitutes the basis of the present invention, that said compounds of the present invention have surprising and advantageous properties.

In particular, said compounds of the present invention have surprisingly been found to effectively inhibit Bradykinin B1 receptor and may therefore be used for the treatment or prophylaxis of following diseases and disorders:
Pain and inflammation, in particular any one of
   visceral pain e.g. related to pancreatitis, interstitial cystitis, renal colic, or prostatitis, chronic pelvic pain, or pain related to infiltrating endometriosis;
   neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
   central pain syndromes potentially caused by virtually any lesion at any level of the nervous system including but not limited to pain related to stroke, multiple sclerosis, and spinal cord injury; and
   postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative and perioperative pain (including but not limited to general surgery, orthopaedic, and gynaecological surgery); and
   acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
and diseases selected from or related to any one of:
   gynaecological disorders and/or diseases, or effects and/or symptoms which negatively influence women health including endometriosis, uterine fibroids, pre-eclampsia, hormonal deficiency, spasms of the uterus, or heavy menstrual bleeding;
   the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
   dermatology including pruritus, itch, inflammatory skin disorders including psoriasis, eczema, and atopic dermatitis;
   affection of the joints or bones including rheumatoid arthritis, gout, osteoporosis, osteoarthritis, and ankylosing spondylitis;
   affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
   infection including HIV infection, and tuberculosis;
   trauma associated with oedema including cerebral oedema, burns, sunburns, and sprains or fracture;
   poisoning including aluminosis, anthracosis, asbestosis, chalicosis, ptilosis, siderosis, silicosis, tabacosis, and byssinosis uveitis, a diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, fat or muscle metabolism,
   cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
   cardio-vascular system including congestive heart failure, atherosclerosis, congestive heart failure, myocardial infarct, and heart fibrosis; and
   a other conditions including primary peritonitis, secondary peritonitis, septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

Additionally, compounds of the present invention reduce the release of inflammation related cytokines like IL-6 and IL-8.

### FIGURE LEGEND

FIG. 1: Dose-response curve of compound example 253 on inhibition of des-Arg9-Bradykinin-induced contractions of bladder stripes from CYP-treated rats

### DETAILED DESCRIPTION OF THE INVENTION

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or sulfur atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “one or a plurality up to the maximum possible amount”, e.g. if the term refers to the carbon atoms of a C₇-cycloalkyl, it relates to “1, 2, 3, 4, 5, 6 or 7”. In particular, “one or more” means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

The term “comprising” when used in the specification includes but is not restricted to “consisting of”.

The terms as mentioned in the present text have preferably the following meanings:

The term “halogen atom”, “halogen”, “halo-” or “Hal-” is to be understood as meaning a fluorine, chlorine, bromine or iodine atom, preferably a fluorine or a chlorine atom.

The term “C₁-C₅-alkyl” means a linear or branched, saturated, monovalent hydrocarbon group having 1, 2, 3, 4 or 5 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl or 1,1-dimethylpropyl group, or an isomer thereof.

The term “C₁-C₃-alkyl” means a linear or branched, saturated, monovalent hydrocarbon group having 1, 2 or 3 carbon atoms (“C₁-C₃-alkyl”), e.g. a methyl, ethyl, n-propyl or isopropyl group.

The term “—OC₁-C₅-alkyl” means a linear or branched, saturated, monovalent group which is attached through an oxygen atom, and in which the term “C₁-C₅-alkyl” is as defined supra, e.g. a methoxy, ethoxy, n-propoxy, isopropoxy, n-butoxy, sec-butoxy, isobutoxy, tert-butoxy, pentyloxy or isopentyloxy, or an isomer thereof. The hyphen at the beginning of the group indicates the point of attachment of said OC₁-C₅-alkyl group to the rest of the molecule.

“C₃-C₇-cycloalkyl” is to be understood as meaning a saturated, monovalent, monocyclic or bicyclic hydrocarbon ring, which contains 3, 4, 5, 6 or 7 carbon atoms. Said C₃-C₇-cycloalkyl group is for example a monocyclic hydrocarbon ring, e.g. a cyclopropyl, cyclobutyl, cyclopentyl, cyclohexyl or cycloheptyl group, or a bicyclic hydrocarbon ring, e.g. a bicyclo[2.2.1]heptanyl or bicyclo[3.2.0]heptanyl group. Particularly, said ring contains 3, 4 or 5 carbon atoms (“C₃-C₅-cycloalkyl”) or 5, 6 or 7 carbon atoms (“C₅-C₇-cycloalkyl”).

The term “bicyclic cycloalkyl” includes by definition spirocycloalkyl, bridged and fused bicycloalkyl groups.

The term “spirocycloalkyl” means a saturated, monovalent bicyclic hydrocarbon group in which the two rings share one common ring carbon atom, and wherein said bicyclic hydrocarbon group contains 5, 6, or 7 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl or spiro[2.4]heptyl.

The term “fused bicycloalkyl” means a bicyclic, saturated hydrocarbon ring with 6 or 7 ring atoms in total, in which the two rings share two adjacent ring atoms.

Said fused cycloalkyl group is, for example, a bicyclo[3.1.0]hexanyl or bicyclo[3.2.0]heptanyl group.

The term “bridged bicycloalkyl” means a bicyclic, saturated hydrocarbon ring with 6 or 7 ring atoms in total, in which the two rings share two common ring atoms which are not adjacent.

Said bridged cycloalkyl group is, for example, bicyclo[2.1.1]hexanyl or bicyclo[2.2.1]heptanyl group.

The term “—(C₁-C₃-alkyl)-(C₃-C₅-cycloalkyl)” is to be understood as a C₃-C₅-cycloalkyl group as defined above which is attached through any carbon atom of said C₃-C₅-cycloalkyl group to any atom of the C₁-C₃-alkyl group as defined above. The hyphen at the beginning of the group indicates the point of attachment of said (C₁-C₃-alkyl)-(C₃-C₅-cycloalkyl) group to the rest of the molecule. Said (C₁-C₃-alkyl)-(C₃-C₅-cycloalkyl) groups are, for example, cyclopropylmethyl, cyclobutylmethyl, cyclopentylmethyl, 2-cyclopropylethyl, 1-cyclopropylethyl, 2-cyclobutylethyl or 1-cyclobutylethyl.

The term “—OC₃-C₅-cycloalkyl” means a saturated, monovalent, monocyclic group, which contains 3, 4 or 5 carbon atoms, in which the term “C₃-C₅-cycloalkyl” is defined supra, e.g. a cyclopropyloxy, cyclobutyloxy or cyclopentyloxy group.

The term “heterocycloalkyl” is to be understood as meaning a saturated, monovalent, monocyclic or bicyclic hydrocarbon ring with the number of ring atoms as specified in which one or two ring atoms of the hydrocarbon ring is/are replaced by one or two heteroatoms or heteroatom-containing groups independently selected from NH, —NR², N, O, S, SO and SO₂, wherein R² represents C₁-C₅-alkyl optionally substituted with 1 to 5 fluorine atoms.

4- to 7-membered heterocycloalkyl in the context of the invention means a monocyclic or bicyclic, saturated heterocycle with 4, 5, 6 or 7 ring atoms in total, which contains one or two identical or different ring heteroatoms or heteroatom-containing groups from the series NH, —NR², N, O, S, SO and SO₂, wherein R² represents C₁-C₅-alkyl optionally substituted with 1 to 5 fluorine atoms. Said 4- to 7-membered heterocycloalkyl can be bound via a ring carbon or nitrogen atom to the rest of the molecule.

Said heterocycloalkyl can be connected through a carbon or a nitrogen atom, if said nitrogen atom is present.

Examples for monocyclic heterocycloalkyl groups are azetidinyl, oxetanyl, thietanyl, pyrrolidinyl, pyrazolidinyl, imidazolidinyl, tetrahydrofuranyl, thiolanyl, 1,1-dioxidothiolanyl, 1,2-oxazolidinyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, 1,2-oxazinanyl, morpholinyl, thiomorpholinyl, 1,1-dioxidothiomorpholinyl, azepanyl, 1,4-diazepanyl, and 1,4-oxazepanyl.

Particularly, without being limited thereto, said heterocycloalkyl can be a 4-membered ring, such as an azetidinyl, oxetanyl or thietanyl, or a 5-membered ring, such as tetrahydrofuranyl, dioxolinyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, or a 7-membered ring, such as a azepanyl, 1,4-diazepanyl or 1,4-oxazepanyl, for example.

The term “bicyclic heterocycloalkyl” includes by definition heterospirocycloalkyl, fused and bridged heterobicycloalkyl groups.

The term “heterospirocycloalkyl” means a bicyclic, saturated heterocycle with 6 or 7 ring atoms in total, in which the two rings share one common ring carbon atom, which “heterospirocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[2.4]heptanyl, azaspiro[3.3]heptyl, oxazaspiro[3.3]heptyl, thiazaspiro[3.3]heptyl, oxaspiro[3.3]heptyl, diazaspiro[3.3]heptyl or thiazaspiro[3.3]heptyl, or one of the further homologous scaffolds such as spiro[2.3]-, spiro[2.4]-, spiro[3.3]-.

The term “fused heterocycloalkyl” means a bicyclic, saturated heterocycle with 6 or 7 ring atoms in total, in which the two rings share two adjacent ring atoms, which “fused heterocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said fused heterocycloalkyl group to be attached to the rest of the molecule via any one of the carbon atoms or, if present, a nitrogen atom.

Said fused heterocycloalkyl group is, for example, 3-azabicyclo[3.1.0]hexanyl or 3-azabicyclo[3.2.0]heptanyl.

The term “bridged heterocycloalkyl” means a bicyclic, saturated heterocycle with 6 or 7 ring atoms in total, in which the two rings share two common ring atoms which are not adjacent, which “bridged heterocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said bridged heterocycloalkyl group to be attached to the rest of the molecule via any one of the carbon atoms, except the bridgehead carbon atoms, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl or diazabicyclo[2.2.1]heptyl.

The term “5- to 7-membered lactam” means cyclic amides of amino carboxylic acids, having a 1-azacycloalkan-2-one structure, or analogues having unsaturation or heteroatoms replacing one or more carbon atoms of the ring having a ring size of 5, 6 or 7 ring system atoms. In particular said “5- to 7-membered lactam” means a γ-lactam (gamma-lactam), a 6-lactam (delta-lactam), and an ε-lactam (epsilon-lactam).

The term “heteroaryl” is understood as meaning a monovalent, monocyclic or bicyclic hydrocarbon ring system with at least one aromatic ring and wherein one, two or three ring atoms of the monovalent, monocyclic or bicyclic hydrocarbon ring system is/are replaced by one, two or three heteroatoms or heteroatom-containing groups independently selected from NH, N, O, S, SO and SO₂. The number of ring system atoms is as specified.

“5- or 6-membered heteroaryl” is understood as meaning a heteroaryl having 5 or 6 ring atoms and wherein one, two or three ring atoms of a monovalent 5-membered hydrocarbon ring system is/are replaced by one, two or three heteroatoms or heteroatom-containing groups independently selected from 5, N, NH and O; and wherein one or two ring atoms of a monovalent 6-membered hydrocarbon ring system is/are replaced by one or two nitrogens.

The said 5-membered heteroaryl can be connected through a carbon or a nitrogen atom, if said nitrogen atom is present.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl or thiadiazolyl; or a 6-membered heteroaryl group, such as, for example, pyridinyl, pyridazinyl, pyrimidinyl or pyrazinyl.

In general, and unless otherwise mentioned, the term “heteroaryl” includes all possible isomeric forms thereof, e.g. tautomers and positional isomers with respect to the point of linkage to the rest of the molecule. Thus, to give some illustrative non-restricting examples, the term pyridinyl includes pyridin-2-yl, pyridin-3-yl, and pyridin-4-yl; or the term pyrimidinyl includes pyrimidin-2-yl, pyrimidin-4-yl and pyrimidin-5-yl; or the term pyrazolyl includes 1H-pyrazolyl; or the term imidazolyl includes 1H-imidazolyl and 4H-imidazolyl; the term thiophenyl includes 2-thiophenyl and 3-thiophenyl; or the term thiazolyl includes 1,3-thiazol-5-yl, 1,3-thiazol-4-yl and 1,3-thiazol-2-yl.

“Bicyclic 8- to 10-membered heteroaryl” is understood as meaning a bicyclic heteroaryl having 8, 9 or 10 ring atoms with at least one aromatic ring and wherein one, two or three ring atoms of a monovalent, 8- to 10-membered bicyclic hydrocarbon ring system is/are replaced by one, two or three heteroatoms or heteroatom-containing groups independently selected from NH, N, O, S, SO and SO₂.

The said bicyclic 8- to 10-membered heteroaryl can be connected through a carbon or a nitrogen atom, if said nitrogen atom is present.

Particularly, bicyclic heteroaryl is selected from for example, benzofuranyl, benzothienyl, benzothiazolyl, thienopyridinyl, thienopyrimidinyl, benzoxazolyl, benzisoxazolyl, benzimidazolyl, benzotriazolyl, benzothiadiazolyl, indazolyl, indolyl, isoindolyl, etc. or for example, quinolinyl, quinazolinyl, isoquinolinyl, etc.; indolizinyl, or cinnolinyl, phthalazinyl, quinazolinyl, quinoxalinyl, etc.

The term “C₁-C₃” as used throughout this text is to be understood as meaning a group having a finite number of carbon atoms of 1 to 3, i.e. 1, 2, or 3 carbon atoms, e.g. in the context of the definition of “C₁-C₃-alkyl”, it is to be understood as meaning an alkyl group having a finite number of carbon atoms of 1 to 3, i.e. 1, 2, or 3 carbon atoms. It is to be understood further that said term “C₁-C₃” is to be interpreted as any sub-range comprised therein, e.g. C₁-C₂, or C₂-C₃.

The term “C₁-C₅” as used throughout this text is to be understood as meaning a group having a finite number of carbon atoms of 1 to 5, i.e. 1, 2, 3, 4, or 5 carbon atoms, e.g. in the context of the definition of “C₁-C₅-alkyl”, it is to be understood as meaning an alkyl group having a finite number of carbon atoms of 1 to 5, i.e. 1, 2, 3, 4, or 5 carbon atoms. It is to be understood further that said term “C₁-C₅” is to be interpreted as any sub-range comprised therein, e.g. C₁-C₅, C₂-C₅, C₃-C₄, C₂-C₃, C₂-C₄, or C₁-C₄.

The term “C₁-C₃” as used in the context of the definition “—OC₁-C₃-alkyl” is to be understood as meaning an alkyl group, having a finite number of carbon atoms of 1 to 3, i.e. 1, 2 or 3 carbon atoms.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy”.

Further, as used herein, the term “C₃-C₇”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, or C₅-C₇; particularly C₃-C₆.

Furthermore, as used herein, the term “C₃-C₅”, as used in the present text, e.g. in the context of the definition of “C₃-C₅-cycloalkyl”, means a cycloalkyl group having a finite number of carbon atoms of 3 to 5, i.e. 3, 4 or 5 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethyl-phenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998, which is incorporated herein by reference.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271] and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases, deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102; both incorporated herein by reference). In other cases, the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208; incorporated herein by reference) and Odanacatib (K. Kassahun et al., WO2012/112363; incorporated herein by reference) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993; incorporated herein by reference). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid.

Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976), thereby incorporated herein.

Further, the compounds of the present invention may exist as tautomers.

The present invention includes all possible tautomers of the compounds of the present invention as single tautomers, or as any mixture of said tautomers, in any ratio.

The term “tetrazolyl” as used in the context of the definition A in the general formula (I) is to be understood as both 1H- and 2H-tautomers.

The present invention also relates to useful forms of the compounds as disclosed herein, such as hydrates, solvates, and salts, in particular pharmaceutically acceptable salts.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19, incorporated herein by reference. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Unless otherwise indicated, the compounds of the present invention are also referred to isomers, enantiomers, diastereomers, racemates, hydrates, solvates, a salt thereof, or a mixture of same.

As used herein, the term “in vivo hydrolysable ester” is understood as meaning an in vivo hydrolysable ester of a compound of the present invention containing a carboxy or hydroxy group, for example, a pharmaceutically acceptable ester that is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₅ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorph, in any ratio.

In accordance with a first aspect, the present invention covers compounds of general formula (I),

wherein
A represents tetrazolyl which is attached to the rest of the molecule by the carbon atom.

In accordance with a further aspect, the present invention covers compounds of general formula (I),

wherein
R¹ represents
   phenyl,
   5- to 7-membered lactam,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 9- or 10-membered heteroaryl containing 1, 2 or 3 heteroatoms or heteroatom-containing groups independently selected from NH, N, O, S, SO and SO₂,
   wherein R¹ is optionally substituted as defined in formula (I).

Also preferred are compounds of general formula (I), wherein
R¹ represents
   phenyl,
   5- to 7-membered lactam,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 9- or 10-membered heteroaryl containing 1, 2 or 3 heteroatoms or heteroatom-containing groups independently selected from NH, N, O, S, SO and SO₂,
      wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
      wherein independently, if R¹ represents 5- to 7-membered lactam, 5-membered heteroaryl or bicyclic 9- or 10-membered heteroaryl, each ring nitrogen atom, if present, of said R¹ is optionally substituted with 1 substituent R^{1b} wherein R^{1b} represents C₁-C₅-alkyl, C₃-C₅-cycloalkyl, or —(C₁-C₃-alkyl)-(C₃-C₅-cycloalkyl), and
      if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl),
      said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents
   6-membered heteroaryl containing 1 or 2 N atoms, in particular pyridinyl, pyrimidinyl or pyrazinyl,
      wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
      wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents pyridinyl, in particular pyridin-3-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein the substituent or at least one of said substituents R^{1a} is preferably positioned para to the carbon atom which links the pyridinyl, in particular pyridin-3-yl, to the rest of the molecule, and
   wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents pyridinyl, in particular pyridin-3-yl, substituted at one or more carbon atoms with 1 or 2 substituents which are the same or different selected from the group consisting of methyl, ethyl, methoxy, ethoxy, trifluoromethyl, difluoromethyl, 1,1-difluoroethyl, 1,1-difluoropropyl and 2,2,2-trifluoroethyl, and
   wherein the substituent or at least one of said substituents is preferably positioned para to the carbon atom which links the pyridinyl, in particular pyridin-3-yl, to the rest of the molecule.

Also preferred are compounds of general formula (I), wherein
R¹ represents
   5-membered heteroaryl, wherein said 5-membered heteroaryl contains 1, 2 or 3 heteroatoms or heteroatom-containing groups independently selected from the group consisting of S, N, NH, and O, in particular pyrazolyl, imidazolyl or thiophenyl,
      wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
      wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents pyrazolyl, in particular pyrazol-4-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently any ring nitrogen atom of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents pyrazol-4-yl, optionally substituted at any ring nitrogen atom with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   wherein said R^{1b} is optionally substituted with one or more substituents independently selected from the group consisting of OH, OR² and F.

Additionally preferred are compounds of general formula (I), wherein
R¹ represents pyrazolyl, in particular pyrazol-4-yl, optionally substituted at a ring nitrogen atom with 1 substituent R^{1b} wherein R^{1b} represents C₁-C₅-alkyl, C₃-C₅-cycloalkyl or —(C₁-C₃-alkyl)-(C₃-C₅-cycloalkyl), and
   wherein said R^{1b} is attached to the pyrazolyl nitrogen atom at position 1, preferably attached to the pyrazol-4-yl nitrogen atom at position 1; and
   wherein said C₁-C₅-alkyl and C₃-C₅-cycloalkyl independently are optionally substituted with one or more substituents independently selected from the group consisting of OH, OR² and F.

Additionally preferred are compounds of general formula (I), wherein
R¹ represents pyrazol-4-yl substituted at any ring nitrogen atom with C₃-C₅-cycloalkyl; and wherein said C₃-C₅-cycloalkyl is unsubstituted or substituted with one or more substituents independently selected from the group consisting of OH, OR² and F.

Also preferred are compounds of general formula (I), wherein
R¹ represents pyrazol-4-yl, optionally substituted at any nitrogen atom with 1 substituent selected from the group consisting of methyl, ethyl, propyl, propan-2-yl, 2-methylpropyl, tertbutyl, butan-2-yl, cyclobutyl, 2,2-dimethylpropyl, 3-methylbutan-2-yl, cyclopentyl, 1-cyclopropylmethyl, 1-cyclopropylethyl, 1-cyclobutylmethyl and 2,2,2-trifluoroethyl, in particular propan-2-yl, 2-methylpropyl, butan-2-yl, cyclobutyl, 2,2-dimethylpropyl, 1-cyclopropylmethyl, 1-cyclopropylethyl, 1-cyclobutylmethyl.

Also preferred are compounds of general formula (I), wherein
R¹ represents pyrazol-4-yl, optionally substituted at any nitrogen atom with cyclobutyl.

Also preferred are compounds of general formula (I), wherein
R¹ represents
   9-membered bicyclic heteroaryl containing 1, 2 or 3 heteroatoms or heteroatom-containing groups independently selected from NH, N, O, S, SO and SO₂, in particular benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents
   benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl, in particular thienopyridynyl, benzopyrazolyl, benzothiazolyl or benzothiophenyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents
   phenyl, pyridinyl, pyrimidinyl, pyrazolyl, imidazolyl, thiophenyl, benzothiophenyl, benzothiazolyl, thienopyridinyl, indolyl or indazolyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently, if R¹ represents pyrazolyl, imidazolyl, benzothiazolyl, thienopyridinyl, indolyl or indazolyl any ring nitrogen atom of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

Also preferred are compounds of general formula (I), wherein
R¹ represents
   pyridinyl, pyrimidinyl, pyrazolyl, thiophenyl, benzothiophenyl, thienopyridinyl, benzothiazolyl, indolyl or indazolyl, in particular pyridinyl, pyrimidinyl or pyrazolyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently, if R¹ represents said pyrazolyl, thienopyridinyl, benzothiazolyl, indolyl or indazolyl, any ring nitrogen atom of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

In accordance with a further aspect, the present invention covers compounds of general formula (I), wherein
R² represents C₁-C₅-alkyl optionally substituted with 1 to 3 fluorine atoms; in particular methyl, ethyl, propyl, or butyl, each optionally substituted with 1 to 3 fluorine atoms.

Also preferred are compounds of general formula (I), wherein
R² represents methyl, difluoromethyl or trifluoromethyl.

In accordance with a further aspect, the present invention covers compounds of general formula (I), wherein
X is CR^{c}.

Also preferred are compounds of general formula (I), wherein
R^{c} represents H, F, Cl or methyl, in particular H or F.

Also preferred are compounds of general formula (I), wherein
R^{c} represents H or F, in particular H.

Also preferred are compounds of general formula (I), wherein
R^{c} represents H or F, in particular F.

In accordance with a further aspect, the present invention covers compounds of general formula (I), wherein
R³ and R⁴ form together a cyclopropyl ring, which is optionally substituted with one or two methyl groups.

Also preferred are compounds of general formula (I), wherein
R³ and R⁴ form together an unsubstituted cyclopropyl ring.

In accordance with a further aspect, the present invention covers compounds of general formula (I), wherein
R⁵ represents
   phenyl,
   —(CH₂)ₚ-(C₅-C₇-cycloalkyl), wherein p is 0 or 1, in particular 0,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
   bicyclic 8- to 10-membered heteroaryl containing 1, 2 or 3 heteroatoms or heteroatom-containing groups independently selected from NH, N, O, S, SO and SO₂,
   wherein R⁵ is optionally substituted as defined in general formula (I).

In accordance with a further aspect, the present invention covers compounds of general formula (I), wherein
p represents 0.

Also preferred are compounds of general formula (I), wherein
R⁵ represents
   phenyl,
   —(CH₂)ₚ-(C₅-C₇-cycloalkyl), wherein p represents 0 or 1, in particular 0,
   6-membered heteroaryl containing 1 or 2 nitrogen atoms, or
   bicyclic 9- or 10-membered heteroaryl containing 1, 2 or 3 heteroatoms or heteroatom-containing groups independently selected from NH, N, O, S, SO and SO₂,
   wherein said R⁵ is optionally substituted at one or more carbon atoms with 1 or 2 substituents which are the same or different and selected from the group consisting of C₁-C₅-alkyl, —OC₁-C₅-alkyl, halogen, OH and CN, wherein said C₁-C₅-alkyl and —OC₁-C₅-alkyl independently are optionally substituted with (a) substituent(s) independently selected from the group consisting of OH, OR²; and 1 to 5 fluorine atoms, and
   wherein independently, if said R⁵ represents bicyclic 9- or 10-membered heteroaryl, each ring nitrogen atom, if present, of said R⁵ is optionally substituted with C₁-C₅-alkyl, which is optionally substituted with OH, OR² or 1 to 5 fluorine atoms.

Also preferred are compounds of general formula (I), wherein
R⁵ represents
   phenyl, or
   6-membered heteroaryl containing 1 or 2 nitrogen atoms,
   wherein said R⁵ is optionally substituted at one or more carbon atoms with 1 or 2 substituents which are the same or different and selected from the group consisting of C₁-C₅-alkyl, OC₁-C₅-alkyl, fluoro and chloro, wherein said C₁-C₅-alkyl and —OC₁-C₅-alkyl independently are optionally substituted with (a) substituent(s) independently selected from the group consisting of OH, OR², and 1 to 5 fluorine atoms.

Also preferred are compounds of general formula (I), wherein
R⁵ represents phenyl optionally substituted with 1 or 2 substituents which are the same or different and selected from the group consisting of C₁-C₅-alkyl, —OC₁-C₅-alkyl, halogen and CN, wherein said C₁-C₅-alkyl and —OC₁-C₅-alkyl independently are optionally substituted with OH, OR² or 1 to 5 fluorine atoms.

Also preferred are compounds of general formula (I), wherein
R⁵ represents phenyl optionally substituted with 1 or 2 substituents which are the same or different and selected from the group consisting of C₁-C₅-alkyl, —OC₁-C₅-alkyl, F and Cl, wherein said C₁-C₅-alkyl and —OC₁-C₅-alkyl independently are optionally substituted with OH, OR² or 1 to 5 fluorine atoms.

Also preferred are compounds of general formula (I), wherein
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is F preferably positioned ortho to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or Cl, said substitutent or said at least one of said substitutents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R⁵ represents phenyl substituted with 2 substituents which are the same or different and selected from the group consisting of C₁-C₅-alkyl, OC₁-C₅-alkyl, F and Cl,
   wherein the substituent or at least one of said substituents is F, preferably positioned ortho to the carbon atom which links the phenyl to the rest of the molecule;
   wherein the other of said substituents is C₁-C₅-alkyl, OC₁-C₅-alkyl or Cl preferably positioned para to the carbon atom which links the phenyl to the rest of the molecule; and
   wherein said C₁-C₅-alkyl and OC₁-C₅-alkyl independently are optionally substituted with 1 to 5 fluorine atoms.

Also preferred are compounds of general formula (I), wherein
R⁵ represents phenyl optionally substituted with 1 or 2 substituents which are the same or different and selected from the group consisting of methyl, trifluoromethyl, trifluoromethoxy, F and Cl,
   wherein if the substituent or at least one of said substituents is F, it is preferably positioned ortho to the carbon atom which links the phenyl to the rest of the molecule, and
   wherein if the substituent or at least one of said substituents is methyl, trifluoromethyl, trifluoromethoxy or Cl, it is preferably positioned para to the carbon atom which links the phenyl to the rest of the molecule.

In accordance with a further aspect, the present invention covers compounds of general formula (I), wherein
R⁵ represents
   6-membered heteroaryl containing 1 or 2 nitrogen atoms, in particular pyridinyl,
   wherein said R⁵ is optionally substituted at one or more carbon atoms with 1 or 2 substituents which are the same or different and selected from the group consisting of C₁-C₅-alkyl, OC₁-C₅-alkyl, OH, halogen and CN, wherein said C₁-C₅-alkyl and OC₁-C₅-alkyl independently are optionally substituted with OH, OR² or 1 to 5 fluorine atoms.

Also preferred are compounds of general formula (I), wherein
R⁵ represents pyridinyl, in particular pyridin-2-yl, optionally substituted at one or more carbon atoms with 1 or 2 substituents which are the same or different and selected from the group consisting of C₁-C₅-alkyl, OC₁-C₅-alkyl, F and Cl, wherein said C₁-C₅-alkyl and OC₁-C₅-alkyl independently are optionally substituted with (a) substituent(s) selected from the group consisting of OH, OR²; and 1 to 5 fluorine atoms.

In accordance with a further aspect, the present invention covers compounds of general formula (I), wherein
R^{d} represents H, OH, F or methyl; in particular H.

In accordance with a further aspect, the present invention covers compounds of general formula (I), wherein
R^{e} represents H or F; in particular H.

In accordance with a further aspect, the present invention covers compounds of general formula (I), wherein
R¹ represents
   phenyl,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 9- or 10-membered heteroaryl containing 1, 2 or 3 heteroatoms or heteroatom-containing groups independently selected from NH, N, O, S, SO and SO₂,
   wherein R¹ is optionally substituted as defined in formula (I);
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents
   phenyl, or
   6-membered heteroaryl containing 1 or 2 N atoms,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN,
   wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and R^{e} represents H or F, in particular H.

In accordance with a further aspect, the present invention covers compounds of general formula (I), wherein
R¹ represents pyridinyl, in particular pyridin-3-yl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein the substituent or at least one of said substituents R^{1a} is preferably positioned para to the carbon atom which links the pyridinyl, in particular pyridin-3-yl, to the rest of the molecule, and
   wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents pyridinyl, in particular pyridin-3-yl, substituted at one or more carbon atoms with 1 or 2 substituents which are the same or different and selected from the group consisting of methyl, ethyl, methoxy, ethoxy, trifluoromethyl, difluoromethyl, 1,1-difluoroethyl, 1,1-difluoropropyl and 2,2,2-trifluoroethyl, and
   wherein the substituent or at least one of said substituents R^{1a} is preferably positioned para to the carbon atom which links the pyridinyl, in particular pyridin-3-yl, to the rest of the molecule;
R³ and R⁴ form together an unsubstituted cyclopropyl ring,
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents
   5-membered heteroaryl, wherein said 5-membered heteroaryl contains 1, 2 or 3 heteroatoms or heteroatom-containing groups independently selected from the group consisting of S, N, NH, and O, in particular pyrazolyl, imidazolyl or thiophenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents pyrazolyl, in particular pyrazol-4-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently said R¹ is optionally substituted at each ring nitrogen atom with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents pyrazol-4-yl, optionally substituted at a nitrogen atom with 1 substituent R^{1b} wherein R^{1b} represents C₁-C₅-alkyl, —(C₁-C₃-alkyl)-(C₃-C₅-cycloalkyl) or C₃-C₅-cycloalkyl, wherein said C₁-C₅-alkyl, C₃-C₅-cycloalkyl and —(C₁-C₃-alkyl)-(C₃-C₅-cycloalkyl) are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents pyrazol-4-yl optionally substituted at each nitrogen atom with 1 substituent selected from the group consisting of methyl, ethyl, propyl, propan-2-yl, 2-methylpropyl, tertbutyl, butan-2-yl, cyclobutyl, 2,2-dimethylpropyl, 3-methylbutan-2-yl and 2,2,2-trifluoroethyl;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents pyrazol-4-yl optionally substituted at any ring nitrogen atom with cyclobutyl;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R¹ represents
   9-membered bicyclic heteroaryl containing 1, 2 or 3 heteroatoms or heteroatom-containing groups independently selected from NH, N, O, S, SO and SO₂, in particular benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

Also preferred are compounds of general formula (I), wherein
R¹ represents
   benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl, in particular thienopyridynyl, benzopyrazolyl, benzothiazolyl or benzothiophenyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
      if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.

In accordance with a further aspect, the present invention covers compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents
   phenyl,
   —(CH₂)ₚ-(C₅-C₇-cycloalkyl), wherein p is 0 or 1, in particular 0,
   6-membered heteroaryl containing 1 or 2 nitrogen atoms, or
   bicyclic 9- or 10-membered heteroaryl containing 1, 2 or 3 heteroatoms or heteroatom-containing groups independently selected from NH, N, O, S, SO and SO₂,
   wherein R⁵ is optionally substituted as defined in general formula (I).

Also preferred are compounds of general formula (I), wherein
R³ and R⁴ form together an unsubstituted cyclopropyl ring, X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents
   phenyl,
   —(CH₂)ₚ-(C₅-C₇-cycloalkyl), wherein p is 0 or 1, in particular 0, or
   6-membered heteroaryl containing 1 or 2 nitrogen atoms,
   wherein R⁵ is optionally substituted as defined in general formula (I).

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents
   phenyl, or
   6-membered heteroaryl containing 1 or 2 nitrogen atoms,
   wherein said R⁵ is optionally substituted at one or more carbon atoms with 1 or 2 substituents which are the same or different and selected from the group consisting of C₁-C₅-alkyl, OC₁-C₅-alkyl, F and Cl, wherein said C₁-C₅-alkyl and —OC₁-C₅-alkyl independently are optionally substituted with OH, OR² or 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl optionally substituted with 1 or 2 substituents which are the same or different and selected from the group consisting of C₁-C₅-alkyl, —OC₁-C₅-alkyl, halogen and CN,
   wherein said C₁-C₅-alkyl and —OC₁-C₅-alkyl independently are optionally substituted with OH, OR² or 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl optionally substituted with 1 or 2 substituents which are the same or different and selected from the group consisting of C₁-C₅-alkyl, —OC₁-C₅-alkyl, F and C₁, wherein said C₁-C₅-alkyl and —OC₁-C₅-alkyl independently are optionally substituted with OH, OR² or 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl substituted with 1 or 2 substituents which are the same or different and selected from the group consisting of C₁-C₅-alkyl, OC₁-C₅-alkyl, F and Cl,
   wherein the substituent or one of said substituents is F, preferably positioned ortho to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substituent or said least one of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl substituted with 2 substituents which are the same or different and selected from the group consisting of C₁-C₅-alkyl, OC₁-C₅-alkyl, F and Cl,
   wherein the substituent or at least one of said substituents is F preferably positioned ortho to the carbon atom which links the phenyl to the rest of the molecule,
   wherein the other of said substituents is C₁-C₅-alkyl, OC₁-C₅-alkyl or C₁, said other of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R³ and R⁴ form together an unsubstituted cyclopropyl ring;
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents phenyl optionally substituted with 1 or 2 substituents which are the same or different and selected from the group consisting methyl, trifluoromethyl, trifluoromethoxy, F and Cl,
   wherein if the substituent or at least one of said substituents is F, it is preferably positioned ortho to the carbon atom which links the phenyl to the rest of the molecule, and
   wherein if the substituent or at least one of said substituents is methyl, trifluoromethyl, trifluoromethoxy or Cl, it is preferably positioned para to the carbon atom which links the phenyl to the rest of the molecule.

In accordance with a further aspect, the present invention covers compounds of general formula (I), wherein
R³ and R⁴ form together an unsubstituted cyclopropyl ring,
X represents CR^{c} or N, in particular CR^{c};
R^{c} represents H, F, Cl, methyl, trifluoromethyl or CN, in particular H or F;
R^{d} represents H or F, in particular H;
R^{e} represents H or F, in particular H; and
R⁵ represents pyridinyl, in particular pyridin-2-yl;
   wherein said R⁵ is optionally substituted at one or more carbon atoms with 1 or 2 substituents which are the same or different and selected from the group consisting of C₁-C₅-alkyl, OC₁-C₅-alkyl, F and Cl, wherein said C₁-C₅-alkyl and OC₁-C₅-alkyl independently are optionally substituted with OH, OR² or 1 to 5 fluorine atoms.

In accordance with a further aspect, the present invention covers compounds of general formula (I), wherein
R¹ represents
   phenyl,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 9- or 10-membered heteroaryl containing 1, 2 or 3 heteroatoms or heteroatom-containing groups independently selected from NH, N, O, S, SO and SO₂,
   wherein R¹ is optionally substituted as defined in formula (I);
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents
   phenyl,
   —(CH₂)ₚ-(C₅-C₇-cycloalkyl), wherein p is 0 or 1, in particular 0, or
   6-membered heteroaryl containing 1 or 2 nitrogen atoms,
   wherein R⁵ is optionally substituted as defined in general formula (I).

In accordance with a further aspect, the present invention covers compounds of general formula (I), wherein
R¹ represents
   6-membered heteroaryl, in particular pyridinyl, pyrimidinyl or pyrazinyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl optionally substituted with 1 or 2 substituents which are the same or different and selected from the group consisting of C₁-C₅-alkyl, —OC₁-C₅-alkyl, halogen and CN, wherein said C₁-C₅-alkyl and —OC₁-C₅-alkyl independently are optionally substituted with OH, OR² or 1 to 5 fluorine atoms.

In accordance with a further aspect, the present invention covers compounds of general formula (I), wherein
R¹ represents pyridinyl, in particular pyridin-3-yl,
   wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein the substituent or at least one of said substituents R^{1a} is preferably positioned para to the carbon atom which links the pyridinyl, in particular pyridin-3-yl, to the rest of the molecule, and
   wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substituent or said at least one of said substituents is preferably positioned para to the carbon atom which links the phenyl to the rest of the molecule; and
   wherein said C₁-C₅-alkyl and OC₁-C₅-alkyl independently are optionally substituted with 1 to 5 fluorine atoms.

Also preferred are compounds of general formula (I), wherein
R¹ represents pyridinyl, in particular pyridin-3-yl, substituted at one or more carbon atoms with 1 or 2 substituents which are the same or different and selected from the group consisting of methyl, ethyl, methoxy, ethoxy, trifluoromethyl, difluoromethyl, 1,1-difluoroethyl, 1,1-difluoropropyl and 2,2,2-trifluoroethyl, and
   wherein the substituent or at least one of said substituents is preferably positioned para to the carbon atom which links the pyridinyl, in particular pyridin-3-yl, to the rest of the molecule;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substituent or said at least one of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

In accordance with a further aspect, the present invention covers compounds of general formula (I), wherein
R¹ represents
   5-membered heteroaryl, wherein said 5-membered heteroaryl contains 1, 2 or 3 heteroatoms or heteroatom-containing groups independently selected from the group consisting of S, N, NH, and O, in particular pyrazolyl, imidazolyl or thiophenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl optionally substituted with 1 or 2 substituents which are the same or different and selected from the group consisting of C₁-C₅-alkyl, —OC₁-C₅-alkyl, halogen and CN, wherein said C₁-C₅-alkyl and —OC₁-C₅-alkyl independently are optionally substituted with OH, OR² or 1 to 5 fluorine atoms.

In accordance with a further aspect, the present invention covers compounds of general formula (I), wherein
R¹ represents
   5-membered heteroaryl, wherein said 5-membered heteroaryl contains 1, 2 or 3 heteroatoms or heteroatom-containing groups independently selected from the group consisting of S, N, NH, and O, in particular pyrazolyl, imidazolyl or thiophenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substituent or said at least one of said substituents is preferably positioned para to the carbon atom which links the phenyl to the rest of the molecule and
   wherein said C₁-C₅-alkyl and OC₁-C₅-alkyl independently are optionally substituted with 1 to 5 fluorine atoms.

In accordance with a further aspect, the present invention covers compounds of general formula (I), wherein
R¹ represents pyrazolyl, in particular pyrazol-4-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently said R¹ is optionally substituted at each ring nitrogen atom with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substituent or said at least one of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R¹ represents pyrazol-4-yl optionally substituted at each ring nitrogen atom with 1 substituent selected from the group consisting of methyl, ethyl, propyl, propan-2-yl, 2-methylpropyl, tertbutyl, butan-2-yl, cyclobutyl, 2,2-dimethylpropyl, 3-methylbutan-2-yl and 2,2,2-trifluoroethyl;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or chloro, said substituent or said at least one of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Also preferred are compounds of general formula (I), wherein
R¹ represents pyrazol-4-yl optionally substituted at any ring nitrogen atom with cyclobutyl;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or chloro, said substituent or said at least one of said substituents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

In accordance with a further aspect, the present invention covers compounds of general formula (I), wherein
R¹ represents
   9-membered bicyclic heteroaryl containing 1, 2 or 3 heteroatoms or heteroatom-containing groups independently selected from NH, N, O, S, SO and SO₂, in particular benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substitutent or said at least one of said substitutents is preferably positioned para to the carbon atom which links the phenyl to the rest of the molecule; and
   wherein said C₁-C₅-alkyl and OC₁-C₅-alkyl independently are optionally substituted with 1 to 5 fluorine atoms.

Also preferred are compounds of general formula (I), wherein
R¹ represents
   benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl, in particular thienopyridynyl, benzopyrazolyl, benzothiazolyl or benzothiophenyl; wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F;
R³ and R⁴ form together an unsubstituted cyclopropyl ring;
R^{d} represents H or F, in particular H;
R^{e} represents H or F, in particular H;
X represents CR^{c} or N, in particular CR^{c};
R^{c} represents H, F, Cl or methyl, in particular H or F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or C₁, said substitutent or said at least one of said substitutents is preferably positioned para to the carbon atom which links the phenyl to the rest of the molecule, and
   wherein said C₁-C₅-alkyl and OC₁-C₅-alkyl independently are optionally substituted with 1 to 5 fluorine atoms.

Preferred compounds are, namely
1-(3-chlorophenyl)-N-[3′,4′-dimethoxy-2-(1H-tetrazol-5-yl)biphenyl-4-yl]cyclopropanecarboxamide;
N-[3′,4′-dimethoxy-2-(1H-tetrazol-5-yl)biphenyl-4-yl]-1-(3-fluoro-4-methylphenyl)cyclopropanecarboxamide;
N-[3′,4′-dimethoxy-2-(1H-tetrazol-5-yl)biphenyl-4-yl]-1-[3-(trifluoromethyl)phenyl]cyclopropanecarboxamide;
1-(4-chlorophenyl)-N-[3′,4′-dimethoxy-2-(1H-tetrazol-5-yl)biphenyl-4-yl]cyclopropanecarboxamide;
N-[3′,4′-dimethoxy-2-(1H-tetrazol-5-yl)biphenyl-4-yl]-1-[4-(trifluoromethyl)phenyl]cyclopropanecarboxamide;
N-[3′,4′-dimethoxy-2-(1H-tetrazol-5-yl)biphenyl-4-yl]-1-(4,4-dimethylcyclohexyl)cyclopropanecarboxamide;
N-[4-(6-ethoxypyridin-3-yl)-3-(1H-tetrazol-5-yl)phenyl]-1-(4-ethylphenyl)cyclopropanecarboxamide;
1-(4-chloro-3-fluorophenyl)-N-[4-(6-ethoxypyridin-3-yl)-3-(1H-tetrazol-5-yl)phenyl]cyclopropanecarboxamide;
1-(3-chloro-4-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[2-fluoro-4-(trifluoromethyl)phenyl]cyclopropanecarboxamide;
1-(3-chloro-2-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[4-(trifluoromethoxy)phenyl]cyclopropanecarboxamide;
1-(4-chloro-2-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-(3-fluoro-4-methylphenyl)cyclopropanecarboxamide;
1-(3-chloro-5-fluorophenyl)-N-[4-(6-ethoxypyridin-3-yl)-3-(1H-tetrazol-5-yl)phenyl]cyclopropanecarboxamide;
1-(3,5-difluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-(3-ethylphenyl)cyclopropanecarboxamide;
N-[4-(6-ethoxypyridin-3-yl)-3-(1H-tetrazol-5-yl)phenyl]-1-(3-propylphenyl)cyclopropanecarboxamide;
N-[4-(6-ethoxypyridin-3-yl)-3-(1H-tetrazol-5-yl)phenyl]-1-[3-(trifluoromethyl)phenyl]cyclopropanecarboxamide;
1-(4-chlorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[4-(trifluoromethyl)phenyl]cyclopropanecarboxamide;
1-(4,4-dimethylcyclohexyl)-N-[4-(6-ethoxypyridin-3-yl)-3-(1H-tetrazol-5-yl)phenyl]cyclopropanecarboxamide;
1-(3-chlorophenyl)-N-[4-(6-propoxypyridin-3-yl)-3-(1H-tetrazol-5-yl)phenyl]cyclopropanecarboxamide;
N-[4-(6-propoxypyridin-3-yl)-3-(1H-tetrazol-5-yl)phenyl]-1-[4-(trifluoromethyl)phenyl]cyclopropanecarboxamide;
1-(2,4-difluorophenyl)-N-[4-(6-propoxypyridin-3-yl)-3-(1H-tetrazol-5-yl)phenyl]cyclopropanecarboxamide;
1-(3-chlorophenyl)-N-[4′-ethoxy-3′-methoxy-2-(1H-tetrazol-5-yl)biphenyl-4-yl]cyclopropanecarboxamide;
1-(3-chlorophenyl)-N-{4-[6-(methoxymethyl)pyridin-3-yl]-3-(1H-tetrazol-5-yl)phenyl}cyclopropanecarboxamide;
1-(3-chlorophenyl)-N-[2-(1H-tetrazol-5-yl)-4′-(trifluoromethyl)biphenyl-4-yl]cyclopropanecarboxamide;
1-(4-chloro-3-fluorophenyl)-N-{3-(1H-tetrazol-5-yl)-4-[6-(trifluoromethyl)pyridin-3-yl]phenyl}cyclopropanecarboxamide;
1-(3-chlorophenyl)-N-{4-[6-(propan-2-yloxy)pyridin-3-yl]-3-(1H-tetrazol-5-yl)phenyl}cyclopropanecarboxamide;
N-{4-[6-(propan-2-yloxy)pyridin-3-yl]-3-(1H-tetrazol-5-yl)phenyl}-1-[4-(trifluoromethyl)phenyl]cyclopropanecarboxamide;
1-(3-chlorophenyl)-N-[4-(5-methoxypyridin-3-yl)-3-(1H-tetrazol-5-yl)phenyl]cyclopropanecarboxamide;
1-(3-chlorophenyl)-N-[4-(6-ethoxy-5-methylpyridin-3-yl)-3-(1H-tetrazol-5-yl)phenyl]cyclopropanecarboxamide;
1-(3-chlorophenyl)-N-[4-(6-ethoxy-5-fluoropyridin-3-yl)-3-(1H-tetrazol-5-yl)phenyl]cyclopropanecarboxamide;
N-[4-(6-ethoxy-5-fluoropyridin-3-yl)-3-(1H-tetrazol-5-yl)phenyl]-1-[4-(trifluoromethyl)phenyl]cyclopropanecarboxamide;
1-(3-chlorophenyl)-N-[4-(2-ethylpyridin-4-yl)-3-(1H-tetrazol-5-yl)phenyl]cyclopropanecarboxamide;
1-[2-fluoro-4-(trifluoromethyl)phenyl]-N-{3-(1H-tetrazol-5-yl)-4-[6-(trifluoromethyl)pyridin-3-yl]phenyl}cyclopropanecarboxamide;
1-(3-chlorophenyl)-N-[4-(6-methylpyridin-3-yl)-3-(1H-tetrazol-5-yl)phenyl]cyclopropanecarboxamide;
1-[2-fluoro-4-(trifluoromethyl)phenyl]-N-[4-(6-methylpyridin-3-yl)-3-(1H-tetrazol-5-yl)phenyl]cyclopropanecarboxamide;
1-(3-chloro-2-fluorophenyl)-N-[4-(2-methylpyridin-4-yl)-3-(1H-tetrazol-5-yl)phenyl]cyclopropanecarboxamide;
1-[2-fluoro-4-(trifluoromethyl)phenyl]-N-[4-(2-methylpyridin-4-yl)-3-(1H-tetrazol-5-yl)phenyl]cyclopropanecarboxamide;
1-(3-chlorophenyl)-N-[4-(1-methyl-1H-indazol-6-yl)-3-(1H-tetrazol-5-yl)phenyl]cyclopropanecarboxamide;
1-(3-chloro-2-fluorophenyl)-N-[4-(1-methyl-1H-indazol-6-yl)-3-(1H-tetrazol-5-yl)phenyl]cyclopropanecarboxamide;
1-[2-fluoro-4-(trifluoromethyl)phenyl]-N-[4-(1-methyl-1H-indazol-6-yl)-3-(1H-tetrazol-5-yl)phenyl]cyclopropanecarboxamide;
N-[2-fluoro-3′,4′-dimethoxy-6-(1H-tetrazol-5-yl)biphenyl-4-yl]-1-[4-(trifluoromethyl)phenyl]cyclopropanecarboxamide;
1-(3-chlorophenyl)-N-[3′-methoxy-2-(1H-tetrazol-5-yl)-4′-(trifluoromethyl)biphenyl-4-yl]cyclopropanecarboxamide;
1-(3-chlorophenyl)-N-[2-fluoro-3′,4′-dimethoxy-6-(1H-tetrazol-5-yl)biphenyl-4-yl]cyclopropanecarboxamide;
1-(3-chlorophenyl)-N-[4-(6-ethoxypyridin-3-yl)-3-fluoro-5-(1H-tetrazol-5-yl)phenyl]cyclopropanecarboxamide;
N-[4-(6-ethoxypyridin-3-yl)-3-fluoro-5-(1H-tetrazol-5-yl)phenyl]-1-[2-fluoro-4-(trifluoromethyl)phenyl]cyclopropanecarboxamide;
N-{3-fluoro-5-(1H-tetrazol-5-yl)-4-[6-(trifluoromethyl)pyridin-3-yl]phenyl}-1-[2-fluoro-4-(trifluoromethyl)phenyl]cyclopropanecarboxamide;
1-(3-chlorophenyl)-N-[3-fluoro-4-(6-methylpyridin-3-yl)-5-(1H-tetrazol-5-yl)phenyl]cyclopropanecarboxamide;
1-(3-chlorophenyl)-N-[3-fluoro-4-(2-methylpyridin-4-yl)-5-(1H-tetrazol-5-yl)phenyl]cyclopropanecarboxamide;
N-[3-chloro-4-(6-ethoxypyridin-3-yl)-5-(1H-tetrazol-5-yl)phenyl]-1-(3-chlorophenyl)cyclopropanecarboxamide;
N-[3-chloro-4-(6-ethoxypyridin-3-yl)-5-(1H-tetrazol-5-yl)phenyl]-1-(3-chloro-2-fluorophenyl)cyclopropanecarboxamide;
1-(3-chlorophenyl)-N-[4-(6-ethoxypyridin-3-yl)-3-(1H-tetrazol-5-yl)phenyl]cyclopropanecarboxamide;
N-{4-[6-(difluoromethyl)pyridin-3-yl]-3-(1H-tetrazol-5-yl)phenyl}-1-[2-fluoro-4-(trifluoromethyl)phenyl]cyclopropanecarboxamide;
1-(3-chlorophenyl)-N-{4-[6-(difluoromethyl)pyridin-3-yl]-3-(1H-tetrazol-5-yl)phenyl}cyclopropanecarboxamide;
1-(3-chlorophenyl)-N-[4-(6-ethoxypyridin-3-yl)-2-hydroxy-5-(1H-tetrazol-5-yl)phenyl]cyclopropanecarboxamide;
N-[3-fluoro-4-(2-methylpyridin-4-yl)-5-(1H-tetrazol-5-yl)phenyl]-1-[2-fluoro-4-(trifluoromethyl)phenyl]cyclopropanecarboxamide;
N-[3-fluoro-4-(6-methylpyridin-3-yl)-5-(1H-tetrazol-5-yl)phenyl]-1-[2-fluoro-4-(trifluoromethyl)phenyl]cyclopropanecarboxamide;
N-{3-fluoro-5-(1H-tetrazol-5-yl)-4-[2-(trifluoromethyl)pyridin-4-yl]phenyl}-1-[2-fluoro-4-(trifluoromethyl)phenyl]cyclopropanecarboxamide;
1-(4-chlorophenyl)-N-{4-[6-(difluoromethyl)pyridin-3-yl]-3-(1H-tetrazol-5-yl)phenyl}cyclopropanecarboxamide;
N-{4-[6-(difluoromethyl)pyridin-3-yl]-3-(1H-tetrazol-5-yl)phenyl}-1-[4-(trifluoromethyl)phenyl]cyclopropanecarboxamide;
N-{4-[6-(difluoromethyl)pyridin-3-yl]-3-(1H-tetrazol-5-yl)phenyl}-1-[4-(trifluoromethoxy)phenyl]cyclopropanecarboxamide;
1-(4-chloro-3-fluorophenyl)-N-{4-[6-(difluoromethyl)pyridin-3-yl]-3-(1H-tetrazol-5-yl)phenyl}cyclopropanecarboxamide;
1-(4-chloro-2-fluorophenyl)-N-{4-[6-(difluoromethyl)pyridin-3-yl]-3-(1H-tetrazol-5-yl)phenyl}cyclopropanecarboxamide;
N-{4-[6-(difluoromethyl)pyridin-3-yl]-3-(1H-tetrazol-5-yl)phenyl}-1-(2-fluoro-4-methylphenyl)cyctopropanecarboxamide;
N-{4-[6-(difluoromethyl)pyridin-3-yl]-3-(1H-tetrazol-5-yl)phenyl}-1-[2-fluoro-4-(trifluoromethoxy)phenyl]cyclopropanecarboxamide;
N-{4-[6-(1,1-difluoroethyl)pyridin-3-yl]-3-(1H-tetrazol-5-yl)phenyl}-1-[4-(trifluoromethyl)phenyl]cyclopropanecarboxamide;
N-{4-[6-(1,1-difluoroethyl)pyridin-3-yl]-3-(1H-tetrazol-5-yl)phenyl}-1-[4-(trifluoromethoxy)phenyl]cyclopropanecarboxamide;
N-{4-[6-(1,1-difluoroethyl)pyridin-3-yl]-3-(1H-tetrazol-5-yl)phenyl}-1-(2-fluoro-4-methylphenyl)cyctopropanecarboxamide;
N-{4-[6-(1,1-difluoroethyl)pyridin-3-yl]-3-(1H-tetrazol-5-yl)phenyl}-1-[2-fluoro-4-(trifluoromethyl)phenyl]cyclopropanecarboxamide;
N-{4-[6-(1,1-difluoroethyl)pyridin-3-yl]-3-(1H-tetrazol-5-yl)phenyl}-1-[2-fluoro-4-(trifluoromethoxy)phenyl]cyclopropanecarboxamide;
1-(3-chlorophenyl)-N-{4-[6-(1,1-difluoropropyl)pyridin-3-yl]-3-(1H-tetrazol-5-yl)phenyl}cyclopropanecarboxamide;
1-(3,4-difluorophenyl)-N-{4-[6-(1,1-difluoropropyl)pyridin-3-yl]-3-(1H-tetrazol-5-yl)phenyl}cyclopropanecarboxamide;
N-{4-[6-(1,1-difluoropropyl)pyridin-3-yl]-3-(1H-tetrazol-5-yl)phenyl}-1-(2-fluoro-4-methylphenyl)cyclopropanecarboxamide;
N-{4-[6-(1,1-difluoropropyl)pyridin-3-yl]-3-(1H-tetrazol-5-yl)phenyl}-1-[2-fluoro-4-(trifluoromethyl)phenyl]cyclopropanecarboxamide;
N-{4-[6-(1,1-difluoropropyl)pyridin-3-yl]-3-(1H-tetrazol-5-yl)phenyl}-1-[2-fluoro-4-(trifluoromethoxy)phenyl]cyclopropanecarboxamide;
1-[3-fluoro-4-(trifluoromethyl)phenyl]-N-{3-(1H-tetrazol-5-yl)-4-[6-(trifluoromethyl)pyridin-3-yl]phenyl}cyclopropanecarboxamide;
1-[2-chloro-4-(trifluoromethyl)phenyl]-N-{3-(1H-tetrazol-5-yl)-4-[6-(trifluoromethyl)pyridin-3-yl]phenyl}cyctopropanecarboxamide;
1-[2-fluoro-4-(trifluoromethoxy)phenyl]-N-{3-(1H-tetrazol-5-yl)-4-[6-(trifluoromethyl)pyridin-3-yl]phenyl}cyctopropanecarboxamide;
N-{3-chloro-5-(1H-tetrazol-5-yl)-4-[6-(trifluoromethyl)pyridin-3-yl]phenyl}-1-[2-fluoro-4-(trifluoromethyl)phenyl]cyclopropanecarboxamide;
1-(3-chlorophenyl)-N-[4-(5-methylpyridin-3-yl)-3-(1H-tetrazol-5-yl)phenyl]cyclopropanecarboxamide;
N-[4-(5-chloro-6-ethoxypyridin-3-yl)-3-(1H-tetrazol-5-yl)phenyl]-1-(3-chlorophenyl)cyclopropanecarboxamide;
1-(3-chlorophenyl)-N-[4-(6-ethylpyridin-3-yl)-3-(1H-tetrazol-5-yl)phenyl]cyclopropanecarboxamide;
1-[2-fluoro-4-(trifluoromethoxy)phenyl]-N-{3-(1H-tetrazol-5-yl)-4-[4-(trifluoromethyl)-1H-pyrazol-1-yl]phenyl}cyclopropanecarboxamide;
N-[4-(4-ethyl-1H-pyrazol-1-yl)-3-(1H-tetrazol-5-yl)phenyl]-1-[2-fluoro-4-(trifluoromethyl)phenyl]cyclopropanecarboxamide;
N-[4-(4-ethyl-1H-pyrazol-1-yl)-3-(1H-tetrazol-5-yl)phenyl]-1-[2-fluoro-4-(trifluoromethoxy)phenyl]cyclopropanecarboxamide;
N-[4-(1-benzothiophen-2-yl)-3-(1H-tetrazol-5-yl)phenyl]-1-[2-fluoro-4-(trifluoromethyl)phenyl]cyclopropanecarboxamide;
1-[2-fluoro-4-(trifluoromethyl)phenyl]-N-{3-(1H-tetrazol-5-yl)-4-[3-(2,2,2-trifluoroethyl)-1H-pyrazol-1-yl]phenyl}cyclopropanecarboxamide;
1-[2-fluoro-4-(trifluoromethoxy)phenyl]-N-{3-(1H-tetrazol-5-yl)-4-[3-(2,2,2-trifluoroethyl)-1H-pyrazol-1-yl]phenyl}cyclopropanecarboxamide;
N-[4-(4-chloro-1H-pyrazol-1-yl)-3-(1H-tetrazol-5-yl)phenyl]-1-[2-fluoro-4-(trifluoromethoxy)phenyl]cyclopropanecarboxamide;
1-[2-fluoro-4-(trifluoromethyl)phenyl]-N-{4-[4-(propan-2-yl)-1H-pyrazol-1-yl]-3-(1H-tetrazol-5-yl)phenyl}cyclopropanecarboxamide;
N-[4-(4-tert-butyl-1H-imidazol-1-yl)-3-(1H-tetrazol-5-yl)phenyl]-1-[2-fluoro-4-(trifluoromethoxy)phenyl]cyclopropanecarboxamide;
N-[4-(4-tert-butyl-1H-pyrazol-1-yl)-3-(1H-tetrazol-5-yl)phenyl]-1-[2-fluoro-4-(trifluoromethyl)phenyl]cyclopropanecarboxamide;
N-[4-(4-tert-butyl-1H-pyrazol-1-yl)-3-(1H-tetrazol-5-yl)phenyl]-1-[2-fluoro-4-(trifluoromethoxy)phenyl]cyclopropanecarboxamide; and
1-[2-fluoro-4-(trifluoromethoxy)phenyl]-N-{4-[4-(propan-2-yl)-1H-pyrazol-1-yl]-3-(1H-tetrazol-5-yl)phenyl}cyclopropanecarboxamide;
   or an isomer, enantiomer, diastereomer, tautomer, racemate, hydrate, solvate, or a salt thereof, or a mixture of same.

It is to be understood that the present invention relates also to any combination of the preferred embodiments described above.

As mentioned above, compounds of the present invention effectively inhibit Bradykinin B1 receptor and may therefore be used for the treatment or prophylaxis of diseases which are related to pain and to inflammation.

Additionally, compounds of the present invention reduce the release of inflammation related cytokines like IL-6 and IL-8.

Pharmaceutical compositions of the compounds of the invention

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations.

For example, the compounds of this invention can be combined with known hormonal therapeutical agents.

In particular, the compounds of the present invention can be administered in combination or as comedication with hormonal contraceptives. Hormonal contraceptives are for example Combined Oral Contraceptives (COCs) or Progestin-Only-Pills (POPs) or hormone-containing devices.

COCs include but are not limited to birth control pills or a birth control method that includes a combination of an estrogen (estradiol) and a progestogen (progestin). The estrogenic part is in most of the COCs ethinyl estradiol. Some COCs contain estradiol or estradiol valerate.

Said COCs contain the progestins norethynodrel, norethindrone, norethindrone acetate, ethynodiol acetate, norgestrel, levonorgestrel, norgestimate, desogestrel, gestodene, drospirenone, dienogest, or nomegestrol acetate.

Birth control pills include for example but are not limited to Yasmin, Yaz, both containing ethinyl estradiol and drospirenone; Microgynon or Miranova containing levonorgestrel and ethinyl estradiol; Marvelon containing ethinyl estradiol and desogestrel; Valette containing ethinyl estradiol and dienogest; Belara and Enriqa containing ethinyl estradiol and chlormadinonacetate; Qlaira containing estradiol valerate and dienogest as active ingredients; and Zoely containing estradiol and normegestrol.

POPs are contraceptive pills that contain only synthetic progestogens (progestins) and do not contain estrogen. They are colloquially known as mini pills.

POPs include but are not limited to Cerazette containing desogestrel; and Micronor containing norethindrone.

Other Progeston-Only forms are intrauterine devices (IUDs), for example Mirena containing levonorgestrel or injectables, for example Depo-Provera containing medroxyprogesterone acetate, or implants, for example Implanon containing etonogestrel.

Other hormone-containing devices with contraceptive effect which are suitable for a combination with the compounds of the present invention are vaginal rings like Nuvaring containing ethinyl estradiol and etonogestrel, or transdermal systems like contraceptive patches, for example Ortho-Evra containing ethinyl estradiol and norelgestromin or Apleek (Lisvy) containing ethinyl estradiol and gestodene.

A preferred embodiment of the present invention is the administration of a compound of general formula (I) in combination with a COC or a POP or other Progestin-Only forms, as well as in combination with vaginal rings or contraceptive patches as mentioned above.

Furthermore, the compounds of the present invention can be combined with therapeutic agents or active ingredients, that are already approved or that are still under development for the treatment and/or prophylaxis of diseases which are related to or mediated by the Bradykinin B1 receptor.

For the treatment and/or prophylaxis of urinary tract diseases, the compounds of the present invention can be administered in combination or as co-medication with any substance that can be applied as therapeutic agent in the following indications: Urinary tract disease states associated with the bladder outlet obstruction; urinary incontinence conditions such as reduced bladder capacity, increased frequency of micturition, urge incontinence, stress incontinence, or bladder hyperreactivity; benign prostatic hypertrophy; prostatic hyperplasia; prostatitis; detrusor hyperreflexia; overactive bladder and symptoms related to overactive bladder wherein said symptoms are in particular increased urinary frequency, nocturia, urinary urgency or urge incontinence; pelvic hypersensitivity; urethritis; prostatitis; prostatodynia; cystitis, in particular interstitial cystitis; idiopathic bladder hypersensitivity.

For the treatment and/or prophylaxis of overactive bladder and symptoms related to overactive bladder, the compounds of the present invention can be administered in combination or as co-medication in addition to behavioural therapy like diet, lifestyle or bladder training with anticholinergics like oxybutynin, tolterodine, propiverine, solifenacin, darifenacin, trospium, fesoterdine; β-3 agonists like mirabegron; neurotoxins like onabutolinumtoxin A; or antidepressants like imipramine, duloxetine.

For the treatment and/or prophylaxis of interstitial cystitis, the compounds of the present invention can be administered in combination or as co-medication in addition to behavioural therapy like diet, lifestyle or bladder training with pentosans like elmiron; antidepressants like amitriptyline, imipramine; or antihistamines like loratadine.

For the treatment and/or prophylaxis of gynaecological diseases, the compounds of the present invention can be administered in combination or as co-medication with any substance that can be applied as therapeutic agent in the following indications: dysmenorrhea, including primary and secondary; dyspareunia; endometriosis; endometriosis-associated pain; endometriosis-associated symptoms, such as and in particular dysmenorrhea, dyspareunia, dysuria, or dyschezia.

For the treatment and/or prophylaxis of dysmenorrhea, including primary and secondary; dyspareunia; endometriosis and endometriosis-associated pain, the compounds of the present invention can be administered in in combination with ovulation inhibiting treatment, in particular COCs as mentioned above or contraceptive patches like Ortho-Evra or Apleek (Lisvy); or with progestogenes like dienogest (Visanne); or with GnRH analogous, in particular GnRH agonists and antagonists, for example leuprorelin, nafarelin, goserelin, cetrorelix, abarelix, ganirelix, degarelix; or with androgens: danazol.

For the treatment and/or prophylaxis of diseases, which are associated with pain, or pain syndromes, the compounds of the present invention can be administered in combination or as co-medication with any substance that can be applied as therapeutic agent in the following indications:

pain-associated diseases or disorders like hyperalgesia, allodynia, functional bowel disorders (such as irritable bowel syndrome) and arthritis (such as osteoarthritis, rheumatoid arthritis and ankylosing spondylitis), burning mouth syndrome, burns, migraine or cluster headache, nerve injury, traumatic nerve injury, post-traumatic injuries (including fractures and sport injuries), neuritis, neuralgia, poisoning, ischemic injury, interstitial cystitis, viral, trigeminal neuralgia, small fiber neuropathy, diabetic neuropathy, chronic arthritis and related neuralgias, HIV and HIV treatment-induced neuropathy.

The compounds of the present invention can be combined with other pharmacological agents and compounds that are intended to treat inflammatory diseases, inflammatory pain or general pain conditions.

In addition to well-known medicaments which are already approved and on the market, the compounds of the present invention can be administered in combination with inhibitors of the P2X purinoceptor family (P2X3, P2X4), with inhibitors of IRAK4 and with antagonists of the prostanoid EP4 receptor.

In particular, the compounds of the present invention can be administered in combination with pharmacological endometriosis agents, intended to treat inflammatory diseases, inflammatory pain or general pain conditions and/or interfering with endometriotic proliferation and endometriosis associated symptoms, namely with inhibitors of Aldo-keto-reductase1C3 (AKR1C3) and with functional blocking antibodies of the prolactin receptor.

The compounds of the present invention can be combined with other pharmacological agents and compounds that are intended for the treatment, prevention or management of cancer.

In particular, the compounds of the present invention can be administered in combination with 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, or zorubicin.

Furthermore, the compounds of the present invention can be combined with active ingredients, which are well known for the treatment of cancer-related pain and chronic pain.

Such combinations include, but are not limited to step II opiods like codeine phosphate, dextropropoxyphene, dihydro-codeine, Tramadol), step III opiods like morphine, fentanyl, buprenorphine, oxymorphone, oxycodone and hydromorphone; and other medications used for the treatment of cancer pain like steroids as Dexamethasone and methylprednisolone; bisphosphonates like Etidronate, Clodronate, Alendronate, Risedronate, and Zoledronate; tricyclic antidepressants like Amitriptyline, Clomipramine, Desipramine, Imipramine and Doxepin; class I antiarrhythmics like mexiletine and lidocaine; anticonvulsants like carbamazepine, Gabapentin, oxcarbazepine, phenytoin, pregabalin, topiramate, alprazolam, diazepam, flurazepam, pentobarbital and phenobarbital.

In addition to those mentioned above, the inventive Bradykinin B1 inhibitors can also be combined with any of the following active ingredients: active ingredients for Alzheimer's therapy, for example acetylcholinesterase inhibitors (e.g. donepezil, rivastigmine, galantamine, tacrine), NMDA (N-methyl-D-aspartate) receptor antagonists (e.g. memantine); L-DOPA/carbidopa (L-3,4-dihydroxyphenylalanine), COMT (catechol-O-methyltransferase) inhibitors (e.g. entacapone), dopamine agonists (e.g. ropinrole, pramipexole, bromocriptine), MAO-B (monoaminooxidase-B) inhibitors (e.g. selegiline), anticholinergics (e.g. trihexyphenidyl) and NMDA antagonists (e.g. amantadine) for treatment of Parkinson's; beta-interferon (IFN-beta) (e.g. IFN beta-1b, IFN beta-1a Avonex® and Betaferon®), glatiramer acetate, immunoglobulins, natalizumab, fingolimod and immunosuppressants such as mitoxantrone, azathioprine and cyclophosphamide for treatment of multiple sclerosis; substances for treatment of pulmonary disorders, for example beta-2-sympathomimetics (e.g. salbutamol), anticholinergics (e.g. glycopyrronium), methylxanthines (e.g. theophylline), leukotriene receptor antagonists (e.g. montelukast), PDE-4 (phosphodiesterase type 4) inhibitors (e.g. roflumilast), methotrexate, IgE antibodies, azathioprine and cyclophosphamide, cortisol-containing preparations; substances for treatment of osteoarthritis such as non-steroidal anti-inflammatory substances (NSAIDs). In addition to the two therapies mentioned, methotrexate and biologics for B-cell and T-cell therapy (e.g. rituximab, abatacept) should be mentioned for rheumatoid disorders such as rheumatoid arthritis and juvenile idiopathic arthritis. Neurotrophic substances such as acetylcholinesterase inhibitors (e.g. donepezil), MAO (monoaminooxidase) inhibitors (e.g. selegiline), interferons und anticonvulsives (e.g. gabapentin); active ingredients for treatment of cardiovascular disorders such as beta-blockers (e.g. metoprolol), ACE inhibitors (e.g. benazepril), diuretics (e.g. hydrochlorothiazide), calcium channel blockers (e.g. nifedipine), statins (e.g. simvastatin); anti-diabetic drugs, for example metformin and glibenclamide, sulphonylureas (e.g. tolbutamide) and insulin therapy for treatment of diabetes and metabolic syndrome. Active ingredients such as mesalazine, sulfasalazine, azathioprine, 6-mercaptopurine or methotrexate, probiotic bacteria (Mutaflor, VSL #3®, Lactobacillus GG, Lactobacillus plantarum, L. acidophilus, L. casei, Bifidobacterium infantis 35624, Enterococcus fecium SF68, Bifidobacterium longum, Escherichia coli Nissle 1917), antibiotics, for example ciprofloxacin and metronidazole, anti-diarrhoea drugs, for example loperamide, or laxatives (bisacodyl) for treatment of chronic-inflammatory bowel disorders. Immunosuppressants such as glucocorticoids and non-steroidale anti-inflammatory substances (NSAIDs), cortisone, chloroquine, cyclosporine, azathioprine, belimumab, rituximab, cyclophosphamide for treatment of lupus erythematosus. By way of example but not exclusively, calcineurin inhibitors (e.g. tacrolimus and ciclosporin), cell division inhibitors (e.g. azathioprine, mycophenolate mofetil, mycophenolic acid, everolimus or sirolimus), rapamycin, basiliximab, daclizumab, anti-CD3 antibodies, anti-T-lymphocyte globulin/anti-lymphocyte globulin for organ transplants, Vitamin D3 analogues, for example calcipotriol, tacalcitol or calcitriol, salicylic acid, urea, ciclosporine, methotrexate, or efalizumab for dermatological disorders.

### Methods of Treating

The present invention relates to a method for using the compounds of the present invention and compositions thereof, to inhibit the Bradykinin B1 receptor.

The present invention relates to a method for using the compounds of the present invention and compositions thereof, to treat mammalian disorders and diseases which include but are not limited to:

Diseases related to pain and inflammation, in particular selected from the group consisting of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
   and diseases like or related to a disease selected from related to the group consisting of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
a diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including primary peritonitis, secondary peritonitis, septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

A preferred embodiment of the present invention relates to a method for using the compounds of the present invention and compositions thereof, to treat a gynaecological disease, preferably dysmenorrhea, dyspareunia or endometriosis, endometriosis-associated pain, or other endometriosis-associated symptoms, wherein said symptoms include dysmenorrhea, dyspareunia, dysuria, or dyschezia. Additionally the present invention relates to a method for using the compounds of the present invention and compositions thereof, to treat osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a gynaecological disease.

### Dose and Administration

Based upon standard laboratory techniques known to evaluate compounds useful for the treatment of disorders and/or diseases which are mediated by Bradykinin B1 receptor, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 20 mg/kg body weight per day. A preferred administration of the compound of the present invention includes but is not limited to 0.1 mg/kg to about 10 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. A preferred oral unit dosage for administration of the compounds of the present invention includes but is not limited to 0.1 mg/kg to about 10 mg/kg body weight one to three times a day to once a week. The average daily dosage for administration by injection, including intravenous, intramuscular, subcutaneous and parenteral injections, and use of infusion techniques will preferably be from 0.01 to 200 mg/kg of total body weight.

The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg of total body weight. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases treated with said method are gynaecological disorders, more preferably dysmenorrhea, dyspareunia or endometriosis, endometriosis-associated pain, or other endometriosis-associated symptoms, wherein said symptoms include dysmenorrhea, dyspareunia, dysuria, or dyschezia. Further diseases which can be treated with said method are osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

Preferably, the method of treating the diseases mentioned above is not limited to the treatment of said disease but also includes the treatment of pain related to or associated with said diseases.

The compounds of the present invention can be used in particular in therapy and prevention, i.e. prophylaxis, of genitourinary, gastrointestinal, respiratory or pain-related disease, condition or disorder.

Methods of testing for a particular pharmacological or pharmaceutical property are well known to persons skilled in the art.

The example testing experiments described herein serve to illustrate the present invention and the invention is not limited to the examples given.

### Synthesis of Compounds of General Formula (I) of the Present Invention

Compounds of general formula (I) with the meaning of R¹, R³, R⁴, R⁵ and A, X, R^{d} and R^{e} as defined in general formula (I), can be synthesised according to various general procedures.

Scheme 1 depicts the synthesis starting from synthons of the formula (II), wherein Hal stands for Cl, Br or I, Br being preferred. The aryl halides of the general formula (II) can be cross-coupled with boronic acids of the general formula (III) or alternatively with their respective pinacol esters to yield compounds of general formula (IV) by Pd-mediated reactions (Suzuki coupling) known to those skilled in the art. A suitable solvent (for example N,N-dimethylformamide, tetrahydrofuran, 1,4-dioxane, dimethoxyethane and optionally water) is used and a base (such as triethylamine, potassium carbonate, caesium carbonate) and a catalyst-ligand mixture, for example of palladium(II) acetate/triphenylphosphine, tetrakis-(triphenylphosphine)palladium(O), bis(triphenylphosphine)-palladium(II) dichloride, bis(diphenylphosphino)ferrocenedichloropalladium (II) is utilised at temperatures between 20° C. and 120° C., preferred at 100° C.

The nitrile moiety of formula (IV) is converted to tetrazoles of the general formula (V) by reaction with 1-4 equivalents of trimethylsilyl azide in the presence of 1-2 equivalents of dibutyltinoxide in toluene or xylene as solvent at temperatures between 50° C. and 160° C. Any tetrazole moieties shown in chemical formulas herein are for illustrative purposes and have to be understood as both 1H- and 2H-tautomers.

Aromatic amines of formula (V) may react with carboxylic acid of formula (VI) by methods known to those skilled in the art to give the compounds of the general formula (I). The reaction is mediated by activating a carboxylic acid of formula (VI) with reagents such as dicyclohexylcarbodiimide (DCC), 1-(3-dimethylaminopropyl)-3-ethylcarbodiimide (EDCI), N-hydroxybenzotriazole (HOBT), N-[(dimethylamino)-(3H-[1,2,3]triazolo[4,5-b]pyridin-3-yloxy)methyliden]-N-methylmethanaminium hexafluorophosphate (HATU) or propylphosphonic anhydride (T3P). For example, the reaction with HATU takes place in an inert solvent, such as N,N-dimethylformamide, dichloromethane or dimethyl sulfoxide in the presence of the appropriate amine formula (V) and a tertiary amine (such as triethylamine or diisopropylethylamine) at temperatures between −30° C. and +60° C.

It is also possible to convert a carboxylic acid of the formula (VI) into the corresponding carboxylic acid chloride with an inorganic acid chloride (such as phosphorus pentachloride, phosphorus trichloride or thionyl chloride) and then into the target compounds of the general formula (I), in pyridine or an inert solvent (such as N,N-dimethylformamide), in the presence of the appropriate amine formula (V) and a tertiary amine (for example triethylamine) at temperatures between −30° C. and +60° C.

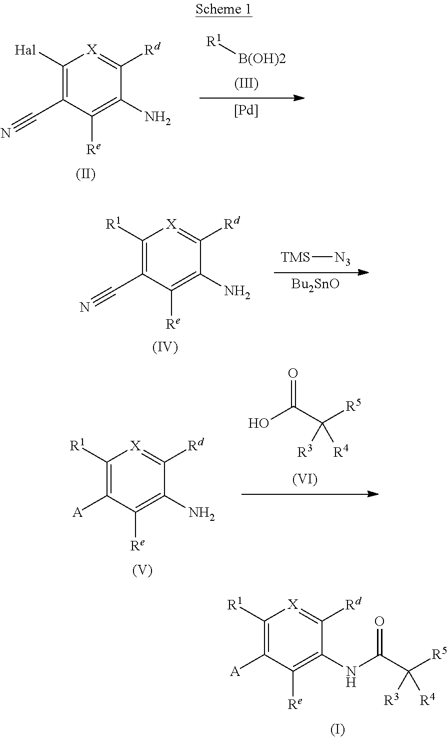

Scheme 2 shows an alternative approach in which the sequence of reaction steps is changed. Also starting from synthons of the general formula (II), first the tetrazole is formed, yielding compounds of the general formula (VII). To the NH of the tetrazole group A, a protecting group is attached. A suitable protecting group is e.g. the 2-(trimethylsilyl)ethoxymethyl group (SEM). To attach the SEM-group, the tetrazole compound (VII) is reacted with 1-1.5 equivalents 2-(trimethylsilyl)ethoxymethyl chloride in the presence of a base, e.g. N,N-diisopropylethylamine (1-2 equivalents) in a solvent like e.g. N,N-dimethylformamide. A separable mixture of both possible SEM-regioisomers is obtained.

In analogy to the procedures described for Scheme 1, amide coupling gives compounds of the general formula (IX), followed by the Suzuki reaction which yields SEM-protected compounds of the general formula (X). Cleavage of the SEM group can be accomplished either by reaction with 1.2 equivalents of tetra-n-butylammonium fluoride in dichloromethane or alternatively by heating to 60° C. in 3 M hydrochloric acid/methanol 1:1 for ca. 1 h, to yield the target compounds of general formula (I).

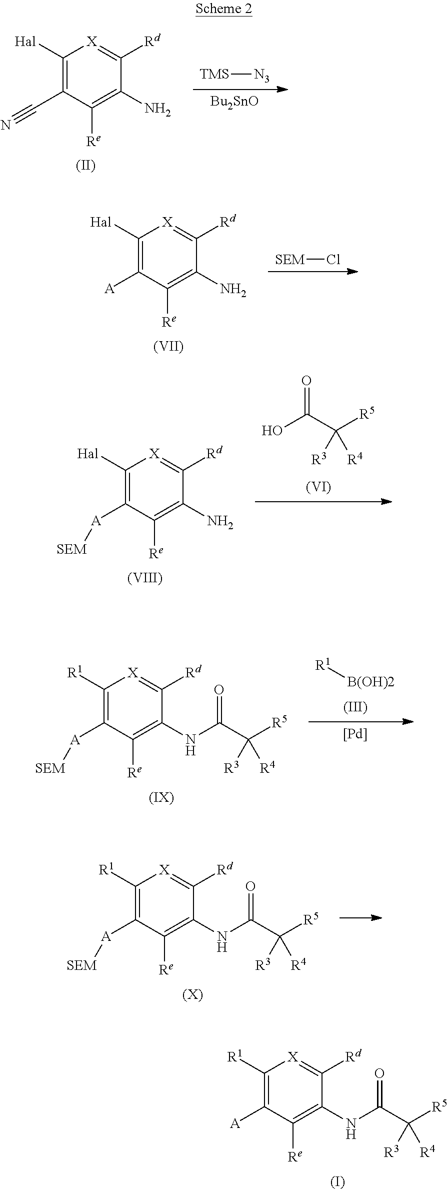

The starting materials of the general formula (II) are either commercially available or can be synthesized via methods known to those skilled in the art from appropriate precursors. For example, the amino group may be obtained by reduction of the corresponding nitro group with hydrogen in the presence of a palladium catalyst in solvents like ethanol, ethyl acetate or mixtures thereof. The nitro group may be introduced by classical methods like treatment with nitric acid/sulphuric acid (with appropriate concentration and volume ratio) at temperatures between 0° C. and 25° C. The sequence of reactions steps (nitro reduction, Suzuki reaction, tetrazole formation) may be changed as appropriate.

The carboxylic acids of the general formula (VI) are either commercially available or can be synthesized via methods known to those skilled in the art from appropriate precursors. For example, arylcyclopropanecarboxylic acids may be prepared from the corresponding arylacetonitrile by cyclopropanation with 1-bromo-2-chloroethane (1.5 eq) in aqueous sodium hydroxide solution in the presence of 0.02 eq. benzyltriethylammonium chloride and subsequent acidic or basic hydrolysis of the nitrile with e.g. lithium hydroxide in water or concentrated hydrochloric acid at temperatures between 20° C. and 100° C.

Scheme 3 shows an alternative approach to synthesise a subset of compounds of general formula (I) wherein R¹ is a substituted cyclohexyl group, herewith defined as compounds of formula (Ia). Starting from synthons of the general formula (XI) (wherein Hal stands for Cl, Br or I; Br being preferred) the aryl halide can first be reacted with a cross-coupling partner of general formula (XII) (wherein X³ is SnBu₃, B(OH)₂ or the respective pinacol boronic ester) to yield a compound of general formula (XIII). A suitable solvent (for example N,N-dimethylformamide, tetrahydrofuran, 1,4-dioxane, dimethoxyethane, toluene and optionally water) is used and a catalyst-ligand mixture, for example of palladium(II) acetate/triphenylphosphine, tetrakis(triphenylphosphine)palladium(O), tris(dibenzylideneacetone)dipalladium, bis(triphenylphosphine)palladium(II) dichloride, bis(diphenylphosphino)ferrocenedichloropalladium (II) is utilised at temperatures between 10° C. and 120° C. The resulting styrene of general formula (XIII) can then be converted into a corresponding cyclohexanone of general formula (XV) by Diels-Alder reaction with a suitable diene (for example 2-trimethylsiloxy-1,3-butadiene) in a suitable solvent (for example toluene or xylene) at temperatures between 10° C. and 180° C. Subsequent hydrolysis of the resulting silyl enol ether is achieved with aqueous acid (i.e. 1-6 molar aqueous hydrochloric acid) where applicable. Reduction of a ketone of general formula (XV) is achieved using a reducing agent (for example sodium borohydride) in a suitable solvent (such as methanol or tetrahydrofuran) at temperatures between −40° C. to 100° C. The resulting alcohol of general formula (XVI) can then be alkylated with an alkyl halide of general formula (XVII) (wherein Hal stands for Cl, Br or I and R^{x} is C₁-C₄-alkyl, optionally substituted with OH, OR^{x} or 1-3 fluorine atoms) in the presence of a suitable base (for example sodium hydride or potassium tert-butoxide) in an appropriate solvent (such as dimethylformamide or dioxane) at temperature between −40° C. and 100° C. The nitrile moiety of formula (XVIII) can subsequently be converted to a tetrazole of the general formula (XIX) by reaction with 1-4 equivalents of trimethylsilyl azide in the presence of 1-2 equivalents of dibutyltinoxide in toluene or xylene solvent at temperatures between 50° C. and 160° C. Any tetrazole moieties shown in chemical formulas herein are for illustrative purposes and have to be understood as both 1H- and 2H-tautomers. The nitro group of a compound of general formula (XIX) is then reduced to the corresponding aniline of general formula (XX) by reaction under a hydrogen atmosphere in the presence of a palladium catalyst (for example 5-10% palladium on carbon) in an appropriate solvent (for example ethanol or ethyl acetate) at temperatures between 0° C. and 100° C. In analogy to the procedures described for Scheme 1, amide coupling gives compounds of the general formula (Ia).

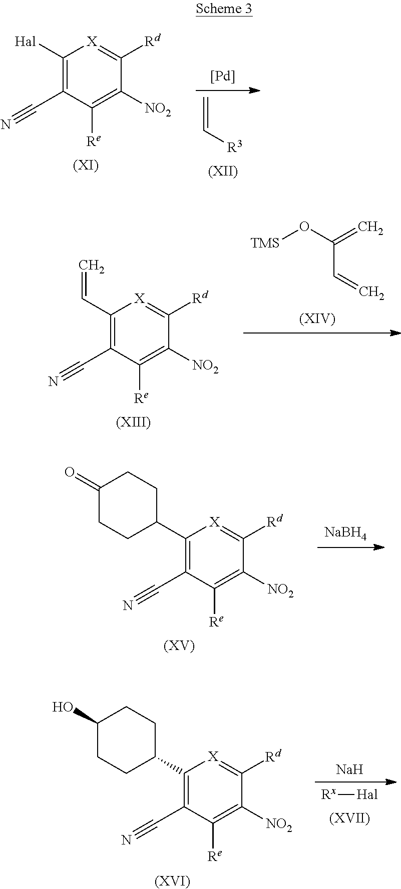

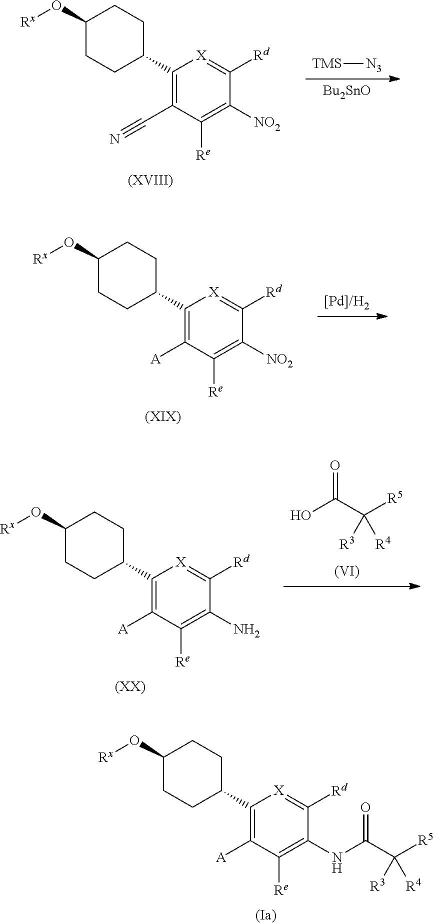

Scheme 4 shows an alternative approach to synthesise a subset of compounds of general formula (I) wherein R¹ is either an N-linked optionally substituted 5-membered heteroaryl group, for example pyrazolyl or imidazolyl, or alternatively R¹ is an N-linked optionally substituted 5- to 7-membered lactam, for example gamma-lactam. Starting from synthons of the general formula (XI) (wherein Hal stands for Cl, Br or I) the aryl halide can first be substituted by a nucleophile of general formula (XXI) to yield a compound of general formula (XXII). The substitution takes place in a dipolar aprotic solvent such as acetonitrile, DMSO or DMF and in the presence of an appropriate base (for example potassium carbonate) at temperatures between RT and 100° C., preferably at 60° C. The nitrile group of formula (XXII) can subsequently be converted to a tetrazole of the general formula (XXIII) by reaction with 1-4 equivalents of trimethylsilyl azide in the presence of 1-2 equivalents of dibutyltinoxide in toluene or xylene solvent at temperatures between 50° C. and 160° C. Any tetrazole moieties shown in chemical formulas herein are for illustrative purposes and have to be understood as both 1H- and 2H-tautomers. The nitro group of a compound of general formula (XXIII) is then reduced to the corresponding aniline of general formula (V) by reaction under a hydrogen atmosphere in the presence of a palladium catalyst (for example 5-10% palladium on carbon) in an appropriate solvent (for example ethanol or ethyl acetate) at temperatures between 0° C. and 100° C. In analogy to the procedures described for Scheme 1, amide coupling gives compounds of the general formula (I).

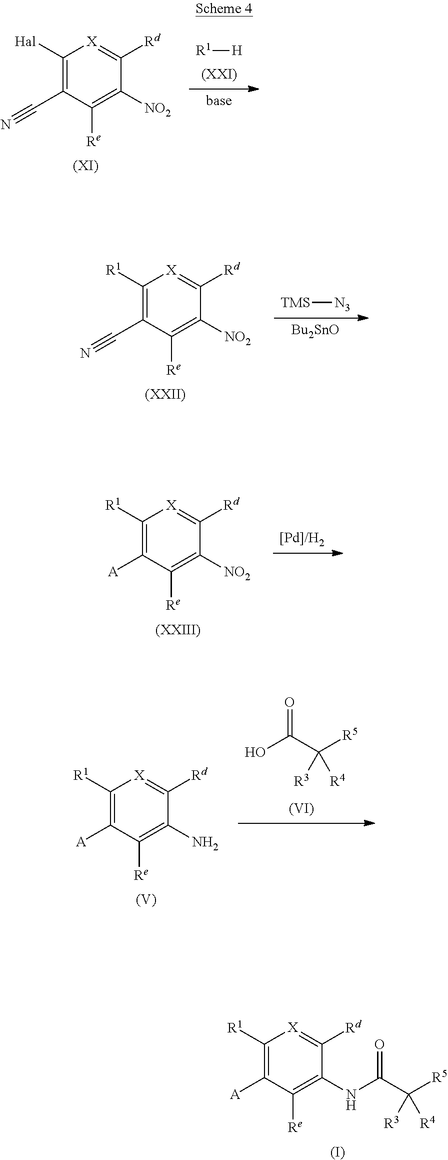

In Scheme 4 general formula XXI represents R¹—H wherein R¹ is an optionally substituted 5- to 7-membered lactam linked through the nitrogen atom or an optionally substituted 5-membered heteroaryl linked through a ring nitrogen atom.

Alternatively to Scheme 4, the reaction sequence can be modified as depicted in Scheme 5 to synthesise compounds of general formula (I), wherein R¹ is either an N-linked optionally substituted 5-membered heteroaryl group, for example pyrazolyl or imidazolyl, or alternatively R¹ is an N-linked optionally substituted 5- to 7-membered lactam, for example gamma-lactam.

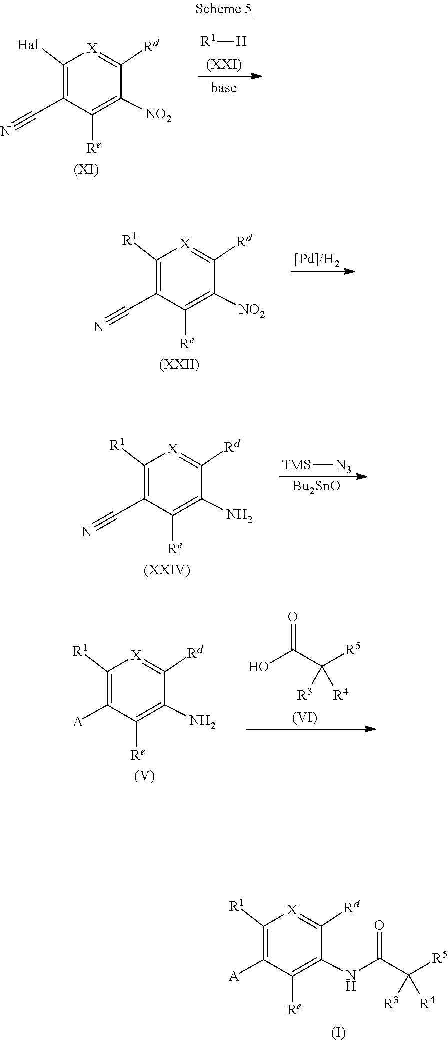

In Scheme 5 general formula XXI represents R¹—H wherein R¹ is an optionally substituted 5- to 7-membered lactam linked through the nitrogen atom or an optionally substituted 5-membered heteroaryl linked through a nitrogen atom to the rest of the molecule.

### EXPERIMENTAL SECTION

The example testing experiments described herein serve to illustrate the present invention and the invention is not limited to the examples given.

The following table lists the abbreviations used in this paragraph, and in the examples section.

| Abbreviation | Meaning |
|---|---|
| Bu₂SnO | Dibutyltinoxide |
| Cs₂CO₃ | Cesium carbonate |
| Cu(I)Cl | Copper(I) chloride |
| ca. | circa |
| DCE | 1,2-Dichloroethane |
| DCM | Dichloromethane |
| DIAD | Diisopropylazodicarboxylate |
| DIPEA | N-Ethyl-N-isopropylpropan-2-amine |
| DMA | Dimethylacetamide |
| DMAP | N,N-Dimethylpyridin-4-amine |
| DME | 1,2-Dimethoxyethane |
| DMF | N,N-Dimethylformamide |
| DMSO | Dimethyl sulfoxide |
| DP | Desired product |
| EE | Ethyl acetate |
| EEDQ | N-Ethoxycarbonyl-2-ethoxy-1,2-dihydroquinoline |
| h | Hour |
| HATU | N-[(Dimethylamino)(3H-[1,2,3]triazolo[4,5- |
| | b]pyridin-3-yloxy)methylene]-N-methylmeth- |
| | anaminium hexafluorophosphate |
| HBr | Hydrogen bromide |
| HCl | Hydrochloric acid |
| HPLC | high performance liquid chromatography |
| IPC | In process check |
| K₂CO₃ | Potassium carbonate |
| LC-MS | liquid chromatography - mass spectrometry |
| LCMS | liquid chromatography - mass spectrometry |
| LiOH | Lithium hydroxide |
| M | Molar |
| μW | Microwave |
| MeCN | Acetonitrile |
| MeOH | Methanol |
| MgSO₄ | Magnesium sulfate |
| min | Minute(s) |
| N | Normal |
| Na₂CO₃ | Sodium carbonate |
| NaH | Sodium hydride |
| NaHCO₃ | Sodium bicarbonate |
| NaI | Sodium iodide |
| NaOH | Sodium hydroxide |
| Na₂SO₄ | Sodium sulfate |
| NH₄Cl | Ammonium chloride |
| NMP | N-Methyl-2-pyrrolidinone |
| NMR | nuclear magnetic resonance spectroscopy |
| PdCl₂(PPh₃)₂ | Bis(triphenylphosphine)palladium(II) dichloride |
| Pd(dppf)Cl₂ | [1,1′-Bis(diphenylphosphino)ferrocene]dichloro- |
| | palladium(II) |
| Pd(dppf)Cl₂•CH₂Cl₂ | 1,1′-Bis(diphenylphosphino)ferrocene-palla- |
| | dium(II)dichloride dichloromethane complex |
| PPh₃ | Triphenylphosphine |
| ppm | parts per million |
| Py | Pyridine |
| RT | Room temperature |
| rt | Retention time |
| Rt | Retention time |
| sat. | Saturated |
| SEM | 2-(trimethylsilyl(ethoxymethyl |
| SM | Starting material |
| STAB | Sodium triacetoxyborohydride |
| HSnBu₃ | Tributyltin hydride |
| TMS-azide | Azidotrimethylsilane |
| TMS-N₃ | Azidotrimethylsilane |
| T3P | Propylphosphonic anhydride |
| TBAB | Tetra-N-butylammonium bromide |
| TBAI | Tetra-N-butylammonium iodide |
| TBME | tert-Butyl methyl ether |
| TEA | Triethylamine |
| TFA | Trifluoroacetic acid |
| THF | Tetrahydrofuran |

### Analysis Methods

### Analytical LCMS Methods

Method 1: Instrument: Waters Acquity Platform ZQ4000; column: Waters BEHC 18, 50 mm×2.1 mm, 1.7 μm; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

Method 2: Instrument: Waters Acquity LCT; column: Phenomenex Kinetex C18, 50 mm×2.1 mm, 2.6 μm; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

Method 3: Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method 4: Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Method 5: Instrument MS: Waters ZQ; instrument HPLC: Waters UPLC Acquity; column: Acquity BEH C18 (Waters), 50 mm×2.1 mm, 1.7 μm; eluent A: water+0, 1% formic acid, eluent B: acetonitrile (Lichrosolv Merck); gradient: 0.0 min 99% A→1.6 min 1% A→1.8 min 1% A→1.81 min 99% A→2.0 min 99% A; oven: 60° C.; flow: 0.800 ml/min; UV-detection PDA 210-400 nm.

### LC-MS, Analytical Method A: Routine High Throughput Analysis

Column: Kinetex Core-Shell C18, 2.1×50 mm, 5 μm; Eluent A: Water+0.1% Formic acid, Eluent B: Acetonitrile+0.1% Formic acid; Gradient 0.00 mins 95% A→1.20 mins 100% B+1.30 mins 100% B+1.31 mins 95% A; column temperature: 40° C.; flow rate 1.2 ml/min; injection volume: 3 μl; UV-detection range: 210-420 nm.

### LC-MS, Analytical Method C: Routine High Throughput Analysis at High pH

Column: Phenomenex Gemini-NX C18, 2.0×50 mm, 3 μm; Eluent A: 2 mM ammonium bicarbonate, buffered to pH10, Eluent B: Acetonitrile; Gradient 0.00 mins 99% A→1.80 mins 100% B+2.10 mins 100% B+2.30 mins 99% A→3.50 mins 99% A; column temperature: 40° C.; flow rate 1.0 ml/min; injection volume: 3 μl; UV-detection range: 210-420 nm.

### LC-MS, Analytical Method D:

Column: Waters Atlantis dC18, 2.1×100 mm, 3 μm; Eluent A: Water+0.1% Formic acid, Eluent B: Acetonitrile+0.1% Formic acid; Gradient 0.00 mins 95% A→5.00 mins 100% B+5.40 mins 100% B+5.42 mins 95% A→7.00 mins 95% A; column temperature: 40° C.; flow rate 0.6 ml/min; injection volume: 3 μl; UV-detection range: 210-420 nm.

### LC-MS, Analytical Method E: High pH

Column: Phenomenex Gemini—NX C18, 2.0×100 mm, 3 μm; Eluent A: 2 mM ammonium bicarbonate, buffered to pH10, Eluent B: Acetonitrile; Gradient 0.00 mins 95% A→5.50 mins 100% B+5.90 mins 100% B+5.92 mins 95% A→7.00 mins 95% A; column temperature: 40° C.; flow rate 0.5 ml/min; injection volume: 3 μl; UV-detection range: 210-420 nm.

### LC-MS, Analytical Method F:

Column: Phenomenex Kinetix-XB C18, 2.1×100 mm, 1.7 μm; Eluent A: Water+0.1% Formic acid, Eluent B: Acetonitrile+0.1% Formic acid; Gradient 0.00 mins 95% A→5.30 mins 100% B+5.80 mins 100% B+5.82 mins 95% A→7.00 mins 95% A; column temperature: 40° C.; flow rate 0.6 ml/min; injection volume: 1 μl; UV-detection range: 200-400 nm.

### Purification Methods:

Biotage Isolera™ chromatography system using pre-packed silica and pre-packed modified silica cartridges.

### Preparative HPLC, Method A: High pH

Column: Waters Xbridge C18, 30×100 mm, 10 μm; Solvent A: Water+0.2% Ammonium hydroxide, Solvent B: Acetonitrile+0.2% Ammonium hydroxide; Gradient 0.00 mins 90% A→0.55 mins 90% A→14.44 mins 95% B+16.55 mins 95% B+16.75 90% A; column temperature: room temperature; flow rate 40 ml/min; injection volume: 1500 μl; Detection: UV 215 nm.

### Preparative HPLC, Method B: Low pH

Column: Waters Sunfire C18, 30×100 mm, 10 μm; Solvent A: Water+0.1% Formic acid, Solvent B: Acetonitrile+0.1% Formic acid; Gradient 0.00 mins 90% A→0.55 mins 90% A→14.44 mins 95% B+16.55 mins 95% B+16.75 90% A; column temperature: room temperature; flow rate 40 ml/min; injection volume: 1500 μl; Detection: UV 215 nm.

### Examples

Chemical naming of the Examples and Intermediates was performed using ACD software by ACD/LABS or Marvin software by ChemAxon.

Reaction times are either specified explicitly in the protocols of the experimental section, or reactions were run until completion. Chemical reactions were monitored and their completion was judged using methods well known to the person skilled in the art, such as thin layer chromatography, e.g. on plates coated with silica gel, or by LCMS methods.

### Intermediate 1A: 4-amino-3′,4′-dimethoxybiphenyl-2-carbonitrile

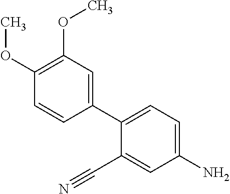 [SMILES: COc1ccc(-c2ccc(N)cc2C#N)cc1OC]

A solution of 5-amino-2-bromobenzonitrile (5.0 g, 25.3 mmol), (3,4-dimethoxyphenyl)boronic acid (5.1 g, 27.8 mmol) and potassium carbonate (11.6 g, 84 mmol) in dimethoxyethane (75 mL) and water (30 mL) was degassed with a stream of nitrogen gas for 5 mins. Dichlorobis(triphenylphosphine)palladium(II) (178 mg, 0.25 mmol) was added, and the reaction heated at 100° C. for 60 min. The reaction was then cooled to RT and diluted with EE (50 mL) and the aqueous layer was removed. The organics were washed with brine (2×40 mL), dried (Na₂SO₄), filtered and concentrated to give the desired product (6.4 g, quant. yield) as an orange solid, which was used without further purification.

¹H NMR (500 MHz, Chloroform-d) 6 [ppm] 7.29 (d, J=8.4 Hz, 1H), 7.08-7.04 (m, 2H), 7.00 (d, J=2.5 Hz, 1H), 6.94 (d, J=8.0 Hz, 1H), 6.91 (dd, J=8.4, 2.5 Hz, 1H), 3.94 (s, 3H), 3.92 (s, 3H), 3.87 (s, 2H); LCMS (Analytical Method A): Rt=1.13 mins; MS (ESIpos) m/z=255 (M+H)⁺.

In analogy to the procedure described for Intermediate 1A, the following intermediates were prepared using 5-amino-2-bromobenzonitrile and the appropriate boronic acids or, respectively, the corresponding pinacol boronic esters as starting materials.

| Int. | Structure | Name | Analytical Data |
|---|---|---|---|
| 2A | 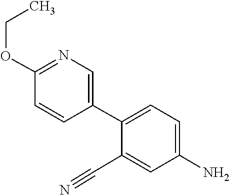 [SMILES: CCOc1ccc(-c2ccc(N)cc2C#N)cn1] | 5-amino-2-(6- ethoxypyridin-3- yl)benzonitrile | ¹H NMR (400 MHz, DMSO-d6) δ [ppm] 1.34 (t, 3H), 4.34 (q, 2H), 5.75 (s, 2H), 6.88 (d, 1H), 6.93 (dd, 1H), 6.98 (d, 1H), 7.27 (d, 1H), 7.81 (dd, 1H), 8.24 (d, 1H). LCMS (method 1): Rₜ = 0.95 mins, MS (ESIpos) m/z = 240 (M + H)⁺. |
| 3A | 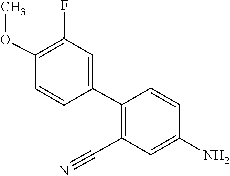 [SMILES: COc1ccc(-c2ccc(N)cc2C#N)cc1F] | 4-amino-3′- fluoro-4′- methoxybiphenyl- 2-carbonitrile | ¹H NMR (500 MHz, DMSO-d6) δ [ppm] 7.35 (s, 1H), 7.26 (m, 3H), 6.95 (d, J = 2.4 Hz, 1H), 6.91 (dd, J = 8.5, 2.5 Hz, 1H), 5.70 (s, 2H), 3.89 (s, 3H). LCMS (Analytical Method A): Rt = 1.23 mins, MS (ESIpos) m/z = 243 (M + H)⁺ |
| 4A | 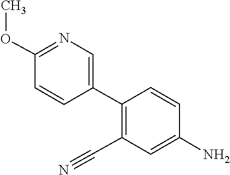 [SMILES: COc1ccc(-c2ccc(N)cc2C#N)cn1] | 5-amino-2-(6- methoxypyridin-3- yl)benzonitrile | ¹H NMR (250 MHz, CDCl₃) δ [ppm] 8.26 (d, J = 2.5 Hz, 1H), 7.75 (dd, J = 8.6, 2.6 Hz, 1H), 7.23 (s, 1H), 7.01 (d, J = 2.5 Hz, 1H), 6.92 (dd, J = 8.4, 2.5 Hz, 1H), 6.82 (d, J = 8.6 Hz, 1H), 3.98 (s, 3H), 3.92 (s, 2H). LCMS (Analytical Method A): Rt = 1.01 mins, MS (ESIpos) m/z = 226 (M + H)⁺. |
| 5A | 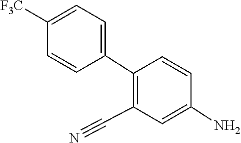 [SMILES: N#Cc1cc(N)ccc1-c1ccc(C(F)(F)F)cc1] | 4-amino-4′- (trifluoromethyl) biphenyl-2- carbonitrile | ¹H NMR (250 MHz, DMSO-d6) δ [ppm] 5.86 (s, 1H), 6.73-7.19 (m, 1H), 7.19-7.54 (m, 1H), 7.54-8.07 (m, 4H). LCMS (Analytical Method A): Rt = 1.25 mins; MS (ESIpos) m/z = 303.95 (M + H)⁺. |
| 6A | 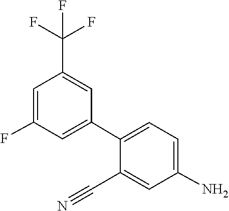 [SMILES: N#Cc1cc(N)ccc1-c1cc(F)cc(C(F)(F)F)c1] | 4-amino-3′- fluoro-5′- (trifluoromethyl) biphenyl-2- carbonitrile | ¹H NMR (400 MHz, DMSO-d6) δ = 5.92 (s, 2H), 6.95 (dd, 1H), 7.01 (d, 1H), 7.40 (d, 1H), 7.70-7.72 (m, 3H). LCMS (method 1): Rt = 1.20 mins, m/z = 281 (M + H)⁺ |
| 7A | 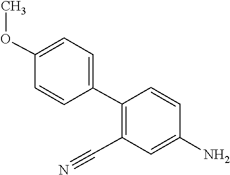 [SMILES: COc1ccc(-c2ccc(N)cc2C#N)cc1] | 4-amino-4′- methoxybiphenyl- 2-carbonitrile | ¹H NMR (500 MHz, DMSO-d6) δ [ppm] 7.44-7.35 (m, 2H), 7.22 (d, J = 8.4 Hz, 1H), 7.06-6.98 (m, 2H), 6.96-6.88 (m, 2H), 5.62 (s, 2H), 3.79 (s, 3H). LCMS (Analytical Method A): Rt = 1.07 mins, MS (ESIpos) m/z = 225 (M + H)⁺. |
| 8A | 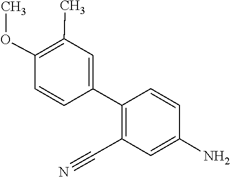 [SMILES: COc1ccc(-c2ccc(N)cc2C#N)cc1C] | 4-amino-4′- methoxy-3′- methylbiphenyl- 2-carbonitrile | ¹H NMR (500 MHz, DMSO-d6) δ [ppm] 2.19 (s, 3H), 3.82 (s, 3H), 5.60 (s, 2H), 6.89 (dd, J = 8.4, 2.4 Hz, 1H), 6.93 (d, J = 2.4 Hz, 1H), 7.00 (d, J = 8.4 Hz, 1H), 7.19-7.28 (m, 3H). LCMS (Analytical Method A): Rt = 1.14 mins, MS (ESIpos) m/z = 239 (M + H)⁺. |
| 9A | 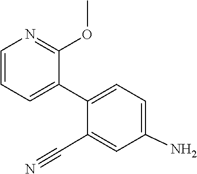 [SMILES: COc1ncccc1-c1ccc(N)cc1C#N] | 5-amino-2-(2- methoxypyridin- 3- yl)benzonitrile | ¹H NMR (400 MHz, DMSO-d6) δ = 3.85 (s, 3H), 5.73 (s, 2H), 6.90 (dd, 1H), 6.95 (d, 1H), 7.09 (dd, 1H), 7.18 (d, 1H). 7.64 (dd, 1H), 8.20 (dd, 1H). LCMS (method 2): Rt = 0.79 mins, m/z = 226 (M + H)⁺ |
| 10A | 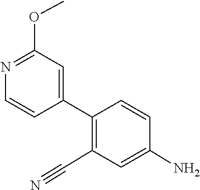 [SMILES: COc1cc(-c2ccc(N)cc2C#N)ccn1] | 5-amino-2-(2- methoxypyridin- 4- yl)benzonitrile | ¹H NMR (500 MHz, DMSO-d6) δ [ppm] 8.22 (d, J = 5.3 Hz, 1H), 7.36 (d, J = 8.5 Hz, 1H), 7.11 (dd, J = 5.3, 1.5 Hz, 1H), 7.00 (d, J = 2.4 Hz, 1H), 6.94 (dd, J = 8.5, 2.4 Hz, 1H), 6.92- 6.89 (m, 1H), 5.91 (s, 2H), 3.89 (s, 3H). LCMS (Analytical Method A): Rt = 1.08 min, MS (ESIpos) m/z = 226 (M + H)⁺. |
| 11A | 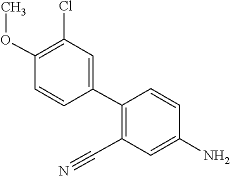 [SMILES: COc1ccc(-c2ccc(N)cc2C#N)cc1Cl] | 4-amino-3′- chloro-4′- methoxybiphenyl-2- carbonitrile | ¹H NMR (250 MHz, CDCl₃) δ [ppm] 7.48 (d, J = 2.3 Hz, 1H), 7.42 (dd, J = 8.5, 2.3 Hz, 1H), 7.24 (d, J = 8.4 Hz, 1H), 7.04-6.97 (m, 2H), 6.90 (dd, J = 8.4, 2.6 Hz, 1H), 3.95 (s, 3H), 3.90 (s, 2H). LCMS (Analytical Method A): Rt = 1.14 min, MS (ESIpos) m/z = 259 (M + H)⁺. |
| 12A | 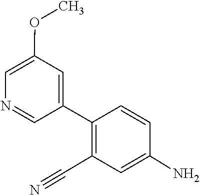 [SMILES: COc1cncc(-c2ccc(N)cc2C#N)c1] | 5-amino-2-(5- methoxypyridin- 3- yl)benzonitrile | ¹H NMR (500 MHz, DMSO-d6) δ [ppm] 8.28 (m, 2H), 7.51-7.44 (m, 1H), 7.36 (d, J = 8.5 Hz, 1H), 7.00 (d, J = 2.4 Hz, 1H), 6.95 (dd, J = 8.5, 2.4 Hz, 1H), 5.82 (s, 2H), 3.88 (s, 3H). LCMS (Analytical Method A): Rt = 0.81 min, MS (ESIpos) m/z = 226 (M + H)⁺. |
| 13A | 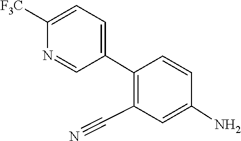 [SMILES: N#Cc1cc(N)ccc1-c1ccc(C(F)(F)F)nc1] | 5-amino-2-[6- (trifluoromethyl) pyridin-3- yl]benzonitrile | ¹H NMR (500 MHz, DMSO-d6) δ [ppm] 5.96 (s, 2H), 6.98 (dd, J = 8.5, 2.4 Hz, 1H), 7.05 (d, J = 2.4 Hz, 1H), 7.43 (d, J = 8.5 Hz, 1H), 8.00 (d, J = 8.2 Hz, 1H), 8.21 (dd, J = 8.2, 2.2 Hz, 1H), 8.88 (d, J = 2.1 Hz, 1H). LCMS (Analytical Method A): Rt = 1.10 min, MS (ESIpos) m/z = 264 (M + H)⁺. |
| 14A | 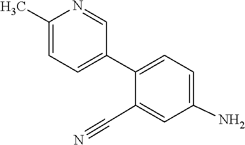 [SMILES: Cc1ccc(-c2ccc(N)cc2C#N)cn1] | 5-amino-2-(6- methylpyridin- 3- yl)benzonitrile | ¹H NMR (500 MHz, DMSO-d6) δ [ppm] 8.56-8.47 (m, 1H), 7.81- 7.73 (m, 1H), 7.38-7.24 (m, 2H), 7.03-6.87 (m, 2H), 5.78 (s, 2H), 3.32 (s, 3H). LCMS (Analytical Method A); Rt = 0.62 min, MS (ESIpos) m/z = 210 (M + H)⁺. |
| 15A | 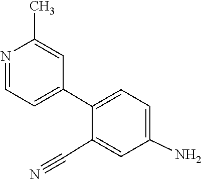 [SMILES: Cc1cc(-c2ccc(N)cc2C#N)ccn1] | 5-amino-2-(2- methylpyridin- 4- yl)benzonitrile | ¹H NMR (500 MHz, DMSO-d6) δ [ppm] 8.51 (d, J = 5.2 Hz, 1H), 7.43-7.30 (m, 3H), 7.01 (d, J = 2.3 Hz, 1H), 6.98-6.91 (m, 1H), 5.93 (s, 2H), 2.52 (s, 3H). |
| 16A | 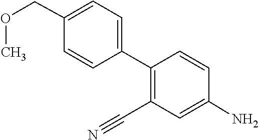 [SMILES: COCc1ccc(-c2ccc(N)cc2C#N)cc1] | 4-amino-4′- (methoxymethyl) biphenyl-2- carbonitrile | ¹H NMR (500 MHz, Methanol-d4) δ [ppm] 7.49 (d, J = 8.2 Hz, 2H), 7.43 (d, J = 8.2 Hz, 2H), 7.29 (d, J = 8.4 Hz, 1H), 7.05 (d, J = 2.4 Hz, 1H), 7.00 (dd, J = 8.4, 2.5 Hz, 1H), 4.53 (s, 2H), 3.42 (s, 3H). LCMS (Analytical Method A): Rt = 1.10 min, MS (ESIpos) m/z = 239 (M + H)⁺. |
| 17A | 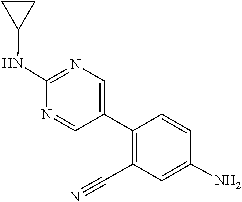 [SMILES: N#Cc1cc(N)ccc1-c1cnc(NC2CC2)nc1] | 5-amino-2-[2- (cyclopropylamino) pyrimidin-5- yl]benzonitrile | ¹H NMR (400 MHz, DMSO-d6) δ [ppm] 0.48-0.52 (m, 2H), 0.67- 0.71 (m, 2H), 2.70-2.77 (m, 1H), 5.72 (s, 2H), 6.93 (dd, 1H), 6.97 (d, 1H), 7.26 (d, 1H), 7.60 (d, 1H), 8.41 (s, 2H). LCMS (method 1): Rₜ = 0.66 mins, MS (ESIpos) m/z = 252 (M + H)⁺. |
| 18A | 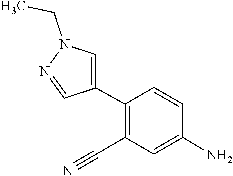 [SMILES: CCn1cc(-c2ccc(N)cc2C#N)cn1] | 5-amino-2-(1- ethyl-1H- pyrazol-4- yl)benzonitrile | ¹H NMR (400 MHz, DMSO-d6) δ [ppm] 1.39 (t, 3H), 4.17 (q, 2H), 5.58 (s, 2H), 6.86-6.89 (m, 2H), 7.34 (d, 1H), 7.76 (s, 1H), 8.05 (s, 1H). LCMS (method 4): Rt = 0.83 mins, MS (ESIpos) m/z = 213 (M + H)⁺. |
| 19A | 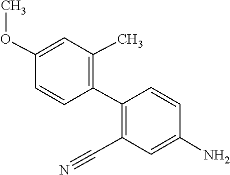 [SMILES: COc1ccc(-c2ccc(N)cc2C#N)c(C)c1] | 4-amino-4′- methoxy-2′- methylbiphenyl- 2-carbonitrile | ¹H NMR (500 MHz, DMSO-d6) δ [ppm] 2.10 (s, 3H), 3.77 (s, 3H), 5.60 (s, 2H), 6.76-6.94 (m, 4H), 7.05 (m, 2H). LCMS (Analytical Method A): Rt = 1.16 min, MS (ESIpos) m/z = 239 (M + H)⁺. |
| 20A | 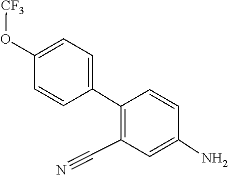 [SMILES: N#Cc1cc(N)ccc1-c1ccc(OC(F)(F)F)cc1] | 4-amino-4′- (trifluoromethoxy) biphenyl-2- carbonitrile | ¹H NMR (500 MHz, DMSO-d6) δ [ppm] 5.78 (s, 2H), 6.84-7.04 (m, 2H), 7.29 (d, J = 8.5 Hz, 1H), 7.45 (d, J = 8.1 Hz, 2H), 7.59 (d, J = 8.6 Hz, 2H). LCMS (Analytical Method A): Rt = 1.20 min, MS (ESIpos) m/z = 279 (M + H)⁺. |
| 21A | 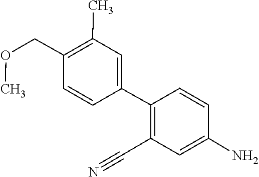 [SMILES: COCc1ccc(-c2ccc(N)cc2C#N)cc1C] | 4-amino-4′- (methoxymethyl)- 3′- methylbiphenyl- 2-carbonitrile | ¹H NMR (500 MHz, DMSO-d6) δ [ppm] 2.31 (s, 3H), 3.34 (s, 3H), 4.44 (s, 2H), 5.68 (s, 2H), 6.91 (dd, J = 8.5, 2.5 Hz, 1H), 6.95 (d, J = 2.4 Hz, 1H), 7.23-7.28 (m, 3H), 7.35 (d, J = 7.9 Hz, 1H). LCMS (Analytical Method A): Rt = 1.12 min, MS (ESIpos) m/z = 253 (M + H)⁺. |
| 22A | 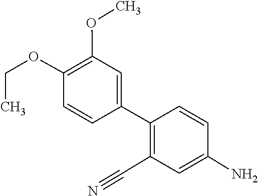 [SMILES: CCOc1ccc(-c2ccc(N)cc2C#N)cc1OC] | 4-amino-4′- ethoxy-3′- methoxybiphenyl- 2-carbonitrile | ¹H NMR (500 MHz, DMSO-d6) δ [ppm] 1.34 (t, J = 7.0 Hz, 3H), 3.80 (s, 3H), 4.04 (q, J = 7.0 Hz, 2H), 5.63 (s, 2H), 6.88-7.03 (m, 4H), 7.05 (d, J = 2.0 Hz, 1H), 7.27 (d, J = 8.4 Hz, 1H). LCMS (Analytical Method A): Rt = 1.08 min, MS (ESIpos) m/z = 269 (M + H)⁺. |
| 23A | 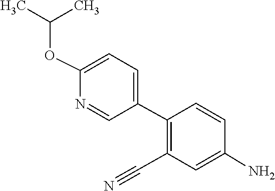 [SMILES: CC(C)Oc1ccc(-c2ccc(N)cc2C#N)cn1] | 5-amino-2-(6- isopropoxypyridin- 3- yl)benzonitrile | ¹H NMR (500 MHz, DMSO-d6) δ [ppm] 1.31 (d, J = 6.2 Hz, 6H), 5.28 (hept, J = 6.2 Hz, 1H), 5.71 (s, 2H), 6.82 (dd, J = 8.6, 0.6 Hz, 1H), 6.92 (dd, J = 8.5, 2.5 Hz, 7.26 (d, J = 8.4 Hz, 1H), 7.78 (dd, J = 8.6, 2.6 Hz, 1H), 8.22 (dd, J = 2.6, 0.6 Hz, 1H). LCMS (Analytical Method A): Rt = 1.14 min, MS (ESIpos) m/z = 254 (M + H)⁺. |
| 24A | 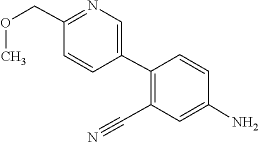 [SMILES: COCc1ccc(-c2ccc(N)cc2C#N)cn1] | 5-amino-2-[6- (methoxymethyl) pyridin-3- yl]benzonitrile | ¹H NMR (250 MHz, DMSO-d6) δ [ppm] 3.39 (s, 3H), 4.54 (s, 2H), 5.81 (s, 2H), 6.86-7.11 (m, 2H), 7.33 (d, J = 8.4 Hz, 1H), 7.41- 7.56 (m, 1H), 7.91 (dd, J = 8.1, 2.4 Hz, 1H), 8.57-8.66 (m, 1H). LCMS (Analytical Method A): Rt = 0.82 min, MS (ESIpos) m/z = 240 (M + H)⁺. |
| 25A | 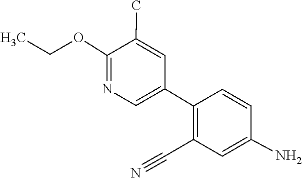 | 5-amino-2-(6- ethoxy-5- methylpyridin- 3- yl)benzonitrile | ¹H NMR (500 MHz, Chloroform-d) δ [ppm] 8.07 (d, J = 2.1 Hz, 1H), 7.55 (dd, J = 2.4, 0.8 Hz, 1H), 7.23 (d, J = 8.4 Hz, 1H), 7.00 (d, J = 2.5 Hz, 1H), 6.91 (dd, J = 8.3, 2.5 Hz, 1H), 4.42 (q, J = 7.1 Hz, 2H), 3.89 (s, 2H), 2.24 (s, 3H), 1.42 (t, J = 7.1 Hz, 3H). LCMS (Analytical Method A): Rt = 1.19 mins; MS (ESIpos) m/z = 254 (M + H)⁺. |
| 26A | 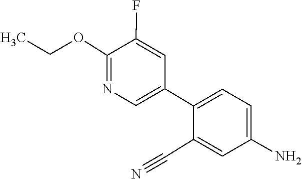 [SMILES: CCOc1ncc(-c2ccc(N)cc2C#N)cc1F] | 5-amino-2-(6- ethoxy-5- fluoropyridin-3- yl)benzonitrile | ¹H NMR (500 MHz, DMSO-d6) δ [ppm] 8.07 (d, J = 2.1 Hz, 1H), 7.83 (dd, J = 11.6, 2.1 Hz, 1H), 7.30 (d, J = 8.5 Hz, 1H), 6.98 (d, J = 2.4 Hz, 1H), 6.92 (dd, J = 8.5, 2.4 Hz, 1H), 5.78 (s, 2H), 4.45 (q, J = 7.0 Hz, 2H), 1.37 (t, J = 7.0 Hz, 3H). LCMS (Analytical Method A): Rt = 1.15 mins; MS (ESIpos) m/z = 258 (M + H)⁺. |
| 27A | 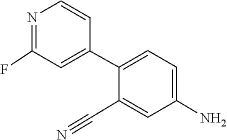 [SMILES: N#Cc1cc(N)ccc1-c1ccnc(F)c1] | 5-amino-2-(2- fluoropyridin-4- yl)benzonitrile | ¹H NMR (400 MHz, DMSO-d6) δ [ppm] 6.04 (s, 2H), 6.96 (dd, 1H), 7.03 (d, 1H), 7.32 (s, 1H), 7.44 (d, 1H), 7.50-7.52 (m, 1H), 8.31 (d, 1H). LCMS (method 1): Rt = 0.76 (ESIpos) m/z = 214 (M + H)⁺. |
| 28A | 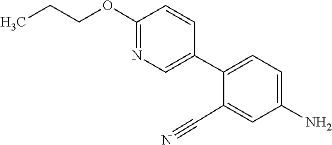 [SMILES: CCCOc1ccc(-c2ccc(N)cc2C#N)cn1] | 5-amino-2-(6- propoxypyridin- 3- yl)benzonitrile | ¹H NMR (250 MHz, Chloroform-d) δ [ppm] 8.24 (dd, J = 2.6, 0.6 Hz, 1H), 7.74 (dd, J = 8.6, 2.6 Hz, 1H), 7.26-7.21 (m, 1H), 7.01 (d, J = 2.4 Hz, 1H), 6.95- 6.89 (m, 1H), 6.80 (dd, J = 8.6, 0.7 Hz, 1H), 4.29 (t, J = 6.7 Hz, 2H), 3.92 (s, 2H), 1.82 (m, 2H), 1.04 (t, J = 7.4 Hz, 3H). |
| | | | LCMS (Analytical Method A): Rt = |
| | | | 1.12 mins; MS (ESIpos) m/z = 254 |
| | | | (M + H)⁺. |
| 29A | 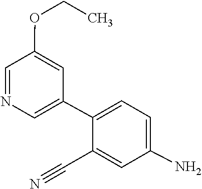 [SMILES: CCOc1cncc(-c2ccc(N)cc2C#N)c1] | 5-amino-2-(5- ethoxypyridin-3- yl)benzonitrile | ¹H NMR (500 MHz, DMSO-d6) δ [ppm] 8.27 (d, J = 2.7 Hz, 1H), 8.25 (d, J = 1.8 Hz, 1H), 7.48- 7.42 (m, 1H), 7.35 (d, J = 8.5 Hz, 1H), 7.00 (d, J = 2.4 Hz, 1H), 6.94 (dd, J = 8.5, 2.4 Hz, 1H), 5.81 (s, 2H), 4.16 (q, J = 7.0 Hz, 2H), 1.36 (t, J = 7.0 Hz, 3H). LCMS (Analytical Method A): Rt = 0.92 mins; MS (ESIpos) m/z = 240 (M + H)⁺. |
| 30A | 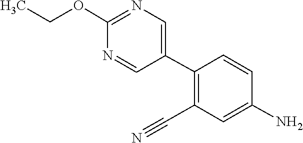 [SMILES: CCOc1ncc(-c2ccc(N)cc2C#N)cn1] | 5-amino-2-(2- ethoxypyrimidin- 5-yl)benzonitrile | ¹H NMR (500 MHz, DMSO-d6) δ [ppm] 8.70 (s, 2H), 7.33 (d, J = 8.4 Hz, 1H), 7.00 (d, J = 2.4 Hz, 1H), 6.95 (dd, J = 8.4, 2.4 Hz, 1H), 5.82 (s, 2H), 4.40 (q, J = 7.0 Hz, 2H), 1.36 (t, J = 7.0 Hz, 3H). LCMS (Analytical Method A): Rt = 1.02 mins; MS (ESIpos) m/z = 240.95 (M + H)⁺. |
| 31A | 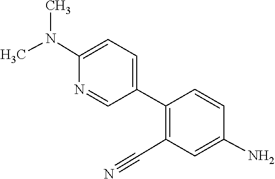 [SMILES: CN(C)c1ccc(-c2ccc(N)cc2C#N)cn1] | 5-amino-2-[6- (dimethylamino) pyridin-3- yl]benzonitrile | ¹H NMR (500 MHz, DMSO-d6) δ [ppm] 8.18 (d, J = 2.4 Hz, 1H), 7.61 (dd, J = 8.8, 2.6 Hz, 1H), 7.22 (d, J = 8.4 Hz, 1H), 6.94 (d, J = 2.4 Hz, 1H), 6.91 (dd, J = 8.4, 2.5 Hz, 1H), 6.70 (d, J = 8.7 Hz, 1H), 5.61 (s, 2H), 3.06 (s, 6H). LCMS (Analytical Method A): Rt = 0.67 mins; MS (ESIpos) m/z = 238.95 (M + H)⁺. |
| 32A | 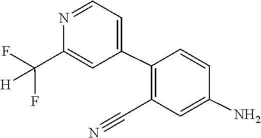 [SMILES: N#Cc1cc(N)ccc1-c1ccnc(C(F)F)c1] | 5-amino-2-[2- (difluoromethyl) pyridin-4- yl]benzonitrile | ¹H NMR (250 MHz, DMSO-d6) δ [ppm] 8.74 (d, J = 5.1 Hz, 1H), 7.84-7.76 (m, 1H), 7.75-7.67 (m, 1H), 7.44 (d, J = 8.5 Hz, 1H), 7.26-6.74 (m, 3H), 6.00 (s, 2H). LCMS (Analytical Method A): Rt = 1.02 mins, MS (ESIpos): m/z = 246 (M + H)⁺ |
| 33A | 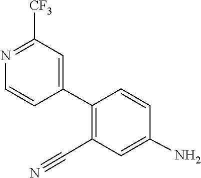 [SMILES: N#Cc1cc(N)ccc1-c1ccnc(C(F)(F)F)c1] | 5-amino-2-[2- (trifluoromethyl) pyridin-4- yl]benzonitrile | ¹H NMR (500 MHz, DMSO-d6) δ [ppm] 8.82 (d, J = 5.1 Hz, 1H), 8.02-7.98 (m, 1H), 7.85 (dd, J = 5.1, 1.4 Hz, 1H), 7.49 (d, J = 8.6 Hz, 1H), 7.05 (d, J = 2.4 Hz, 1H), 6.97 (dd, J = 8.6, 2.4 Hz, 1H), 6.05 (s, 2H). LCMS (Analytical Method A): Rt = 1.19 mins; MS (ESIpos) m/z = 263.90 (M + H)⁺. |
| 34A | 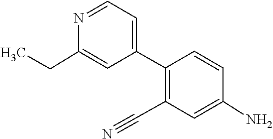 [SMILES: CCc1cc(-c2ccc(N)cc2C#N)ccn1] | 5-amino-2-(2- ethylpyridin-4- yl)benzonitrile | LCMS (method 4): Rt = 0.90 min, MS (ESIpos) m/z = 224 (M + H)⁺. |
| 35A | 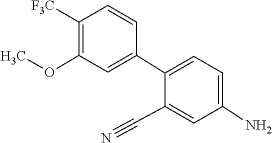 [SMILES: COc1cc(-c2ccc(N)cc2C#N)ccc1C(F)(F)F] | 4-amino-3′- methoxy-4′- (trifluoromethyl) biphenyl-2- carbonitrile | LCMS (method 3): Rt = 1.21 min, MS (ESIpos) m/z = 293 (M + H)⁺. |
| 36A | 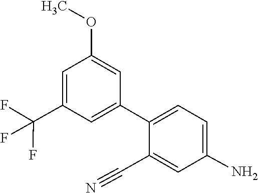 [SMILES: COc1cc(-c2ccc(N)cc2C#N)cc(C(F)(F)F)c1] | 4-amino-3′- methoxy-5′- (trifluoromethyl) biphenyl-2- carbonitrile | ¹H NMR (400 MHz, DMSO-d6) δ [ppm] 3.89 (s, 3H), 5.85 (s, 2H), 6.94 (dd, 1H), 7.00 (d, 1H), 7.25 (m, 1H), 7.34-7.38 (m, 3H). LCMS (method 1): Rt = 1.19 min, MS (ESIpos) m/z = 293 (M + H)⁺. |
| 37A | 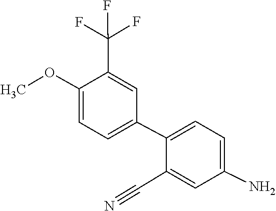 [SMILES: COc1ccc(-c2ccc(N)cc2C#N)cc1C(F)(F)F] | 4-amino-4′- methoxy-3′- (trifluoromethyl) biphenyl-2- carbonitrile | ¹H NMR (400 MHz, DMSO-d6) δ [ppm] 3.94 (s, 3H), 5.75 (s, 2H), 6.93 (dd, 1H), 6.97 (d, 1H), 7.30 (d, 1H), 7.37 (d, 1H), 7.67 (d, 1H), 7.75 (dd, 1H). LCMS (method 1): Rt = 1.13 min, MS (ESIpos) m/z = 293 (M + H)⁺. |
| 38A | 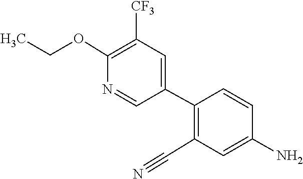 [SMILES: CCOc1ncc(-c2ccc(N)cc2C#N)cc1C(F)(F)F] | 5-amino-2-[6- ethoxy-5- (trifluoromethyl) pyridin-3- yl]benzonitrile | ¹H NMR (500 MHz, DMSO-d6) δ [ppm] 8.52 (d, J = 2.3 Hz, 1H), 8.16 (d, J = 2.3 Hz, 1H), 7.36 (d, J = 8.5 Hz, 1H), 7.00 (d, J = 2.4 Hz, 1H), 6.94 (dd, J = 8.5, 2.4 Hz, 1H), 5.81 (s, 2H), 4.51 (q, J = 7.0 Hz, 2H), 1.36 (t, J = 7.0 Hz, 3H). LCMS (Analytical Method A): Rt = 1.34 mins; MS (ESIpos) m/z = 307.95 (M + H)⁺. |
| 39A | 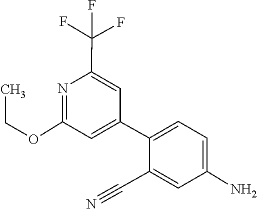 [SMILES: CCOc1cc(-c2ccc(N)cc2C#N)cc(C(F)(F)F)n1] | 5-amino-2-[2- ethoxy-6- (trifluoromethyl) pyridin-4- yl]benzonitrile | LCMS (method 4): Rt = 1.31 min, MS (ESIpos) m/z = 308 (M + H)⁺. |
| 40A | 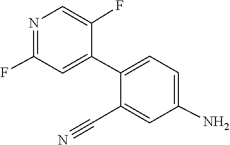 [SMILES: N#Cc1cc(N)ccc1-c1cc(F)ncc1F] | 5-amino-2-(2,5- difluoropyridin- 4- yl)benzonitrile | LCMS (method 1): Rt = 0.83 min, MS (ESIpos) m/z = 232 (M + H)⁺. |
| 41A | 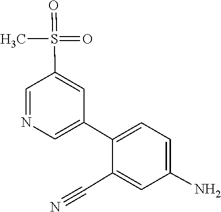 [SMILES: CS(=O)(=O)c1cncc(-c2ccc(N)cc2C#N)c1] | 5-Amino-2-[5- (methylsulfonyl) pyridin-3- yl]benzonitrile | LCMS (method 2): Rt = 0.60 min, MS (ESIpos) m/z = 274 (M + H)⁺. |
| 42A | 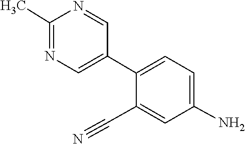 [SMILES: Cc1ncc(-c2ccc(N)cc2C#N)cn1] | 5-amino-2-(2- methylpyrimidin- 5- yl)benzonitrile | ¹H NMR (250 MHz, DMSO-d6) δ [ppm] 8.83 (s, 2H), 7.38 (d, J = 8.4 Hz, 1H), 7.05-6.91 (m, 2H), 5.89 (s, 2H), 2.68 (s, 3H). LCMS (Analytical Method A): Rt = 0.86 mins; MS (ESIpos) m/z = 210.9 (M + H)⁺. |
| 43A | 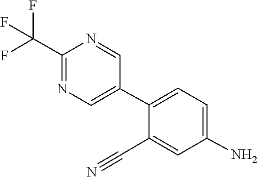 [SMILES: N#Cc1cc(N)ccc1-c1cnc(C(F)(F)F)nc1] | 5-Amino-2-[2- (trifluoromethyl) pyrimidin-5- yl]benzonitrile | ¹H NMR (500 MHz, Chloroform-d) δ [ppm] 9.05 (s, 2H), 7.32 (d, J = 8.5 Hz, 1H), 7.09 (d, J = 2.5 Hz, 1H), 7.00 (dd, J = 8.5, 2.5 Hz, 1H), 4.18 (s, 2H). LCMS (Analytical Method A): Rt = 1.22 mins; MS (ESIpos) m/z = 264.95 (M + H)⁺. |
| 44A | 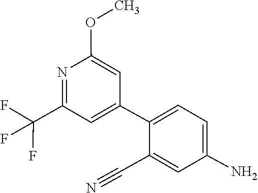 [SMILES: COc1cc(-c2ccc(N)cc2C#N)cc(C(F)(F)F)n1] | 5-amino-2-[2- methoxy-6- (trifluoromethyl) pyridin-4- yl]benzonitrile | ¹H NMR (500 MHz, DMSO-d6) δ [ppm] 7.59 (d, J = 1.2 Hz, 1H), 7.46 (d, J = 8.6 Hz, 1H), 7.41 (d, J = 8.8 Hz, 1H), 7.24 (s, 1H), 7.06-7.01 (m, 1H), 6.02 (s, 2H), 3.94 (s, 3H) LCMS (Analytical Method A): Rt = 1.23 mins; MS (ESIpos) m/z = 294 (M + H)⁺. |
| 45A | 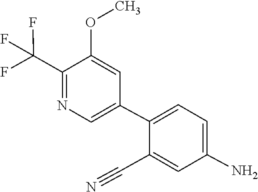 [SMILES: COc1cc(-c2ccc(N)cc2C#N)cnc1C(F)(F)F] | 5-amino-2-[5- methoxy-6- (trifluoromethyl) pyridin-3- yl]benzonitrile | ¹H NMR (400 MHz, DMSO-d6) δ [ppm] 4.00 (s, 3H), 5.99 (s, 2H), 6.99 (dd, 1H), 7.05 (d, 1H), 7.48 (d, 1H), 7.90 (d, 1H), 8.38 (d, 1H). LCMS (method 2): Rt = 0.97 min; MS (ESIpos) m/z = 294 (M + H)⁺. |
| 46A | 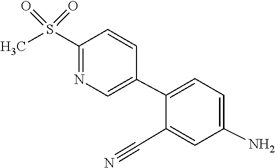 [SMILES: CS(=O)(=O)c1ccc(-c2ccc(N)cc2C#N)cn1] | 5-amino-2-[6- (methylsulfonyl) pyridin-3- yl]benzonitrile | ¹H NMR (500 MHz, DMSO-d6) δ [ppm] 8.92-8.86 (m, 1H), 8.26 (dd, J = 8.2, 2.3 Hz, 1H), 8.12 (dd, J = 8.2, 0.6 Hz, 1H), 7.43 (d, J = 8.5 Hz, 1H), 7.05 (d, J = 2.4 Hz, 1H), 6.98 (dd, J = 8.5, 2.4 Hz, 1H), 5.99 (s, 2H), 3.34 (s, 3H). LCMS (Analytical Method A): Rt = 0.93 min; MS (ESIpos) m/z = 274 (M + H)⁺. |
| 47A | 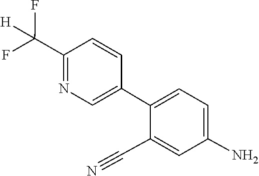 [SMILES: N#Cc1cc(N)ccc1-c1ccc(C(F)F)nc1] | 5-amino-2-[6- (difluoromethyl) pyridin-3- yl)benzonitrile | ¹H NMR (500 MHz, DMSO-d6) δ [ppm] 8.79 (d, J = 2.1 Hz, 1H), 8.11 (dd, J = 8.1, 2.3 Hz, 1H), 7.79 (d, J = 8.1 Hz, 1H), 7.39 (d, J = 8.5 Hz, 1H), 7.03 (d, J = 2.4 Hz, 1H), 7.01 (t, J = 55.0 Hz, 1H), 6.97 (dd, J = 8.5, 2.4 Hz, 1H), 5.90 (s, 2H). LCMS (Analytical Method A): Rt = 1.12 mins; MS (ESIpos) m/z = 246 (M + H)⁺. |
| 48A | 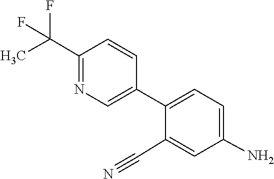 [SMILES: CC(F)(F)c1ccc(-c2ccc(N)cc2C#N)cn1] | 5-amino-2-[6- (1,1- difluoroethyl) pyridin-3- yl]benzonitrile | ¹H NMR (250 MHz, DMSO-d6) δ [ppm] 8.77 (d, J = 1.6 Hz, 1H), 8.15-8.01 (m, 1H), 7.79 (dd, J = 8.2, 0.7 Hz, 1H), 7.38 (d, J = 8.5 Hz, 1H), 7.11-6.87 (m, 2H), 5.89 (s, 2H), 2.04 (t, J = 19.1 Hz, 3H). LCMS (Analytical Method A): Rt = 1.07 mins; MS (ESIpos) m/z = 260.0 (M + H)⁺. |
| 49A | 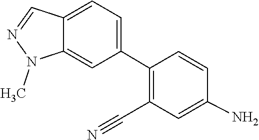 [SMILES: Cn1ncc2ccc(-c3ccc(N)cc3C#N)cc21] | 5-amino-2-(1- methyl-1H- indazol-6- yl)benzonitrile | ¹H NMR (500 MHz, DMSO-d6) δ [ppm] 8.07 (s, 1H), 7.80 (s, 1H), 7.69 (s, 1H), 7.36 (d, J = 8.4 Hz, 1H), 7.26-7.22 (m, 1H), 7.01 (d, J = 2.4 Hz, 1H), 6.99-6.93 (m, 1H), 5.74 (s, 2H), 4.06 (s, 3H) LCMS (Analytical Method A): Rt = 1.05 mins; MS (ESIpos) m/z = 249 (M + H)⁺. |
| 50A | 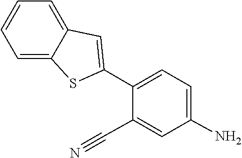 [SMILES: N#Cc1cc(N)ccc1-c1cc2ccccc2s1] | 5-amino-2-(1- benzothiophen- 2- yl)benzonitrile | ¹H NMR (500 MHz, DMSO-d6) δ [ppm] 5.96 (s, 2H), 6.95 (dd, 1H), 7.02 (d, 1H), 7.40 (m, 2H), 7.47 (d, 1H), 7.67 (s, 1H), 7.88 (d, 1H), 7.98 (d, 1H). LCMS (Analytical Method A) Rt = 1.21 min; MS (ESIpos) m/z = 250.9 (M + H)⁺. |
| 51A | 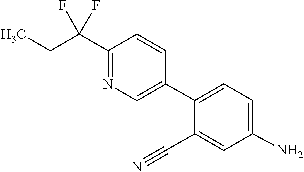 [SMILES: CCC(F)(F)c1ccc(-c2ccc(N)cc2C#N)cn1] | 5-amino-2-[6- (1,1- difluoropropyl) yl]benzonitrile | ¹H NMR (500 MHz, DMSO-d6) δ 8.78 (d, J = 1.9 Hz, 1H), 8.09 (dd, J = 8.2, 2.3 Hz, 1H), 7.77 (d, J = 8.2 Hz, 1H), 7.39 (d, J = 8.5 Hz, 1H), 7.03 (d, J = 2.4 Hz, 1H), 6.97 (dd, J = 8.5, 2.4 Hz, 1H), 5.89 (s, 2H), 2.42-2.29 (m, 2H), 0.95 (t, J = 7.5 Hz, 3H). LCMS (Analytical Method A) Rt = 1.13 min; MS (ESIpos) m/z = 274 (M + H)⁺. |

### Intermediate 52A: 5-amino-6′-(trifluoromethyl)-2,3′-bipyridine-3-carbonitrile

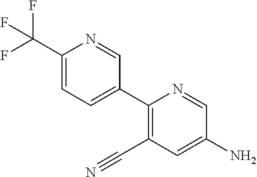 [SMILES: N#Cc1cc(N)cnc1-c1ccc(C(F)(F)F)nc1]

2-Chloro-5-nitronicotinonitrile (870 mg, 4.74 mmol) was dissolved in ethanol/ethyl acetate (20 mL, 2:1) at room temperature. Palladium on carbon (10%, 100 mg) was added and the reaction stirred under an atmosphere of hydrogen at room temperature for 6 hours. The reaction mixture was filtered through a Celite® pad, washed with ethyl acetate and the filtrate concentrated under reduced pressure to give 5-amino-2-chloronicotinonitrile (620 mg, 85% yield).

LCMS (method 4): Rt=0.72, MS (ESipos) m/z=154 (M+H)⁺.

To 5-amino-2-chloronicotinonitrile (205 mg, 1.34 mmol), 2-trifluoromethylpyridine-5-boronic acid (510 mg, 2.67 mmol), potassium carbonate (610 mg, 4.41 mmol) and dichlorobis(triphenylphosphine) palladium(II) (10 mg, 13 μmol) was added degassed water (2 mL) and 1,2-dimethoxyethane (5 mL). The mixture was stirred for 3 h at 90° C. Further 2-trifluoromethylpyridine-5-boronic acid (250 mg) and dichlorobis(triphenylphosphine) palladium(II) (9 mg) were added and the mixture heated to 90° C. for further 2 h and stirred at RT for further 16 h. The cooled reaction mixture was poured into water and extracted with ethyl acetate (2×) and the combined organic layer washed with brine, dried (Na₂SO₄), filtered and concentrated under reduced pressure. The desired title compound was used without further purification.

LCMS (method 4): Rt=0.99 min, MS (ESipos) m/z=265 (M+H)⁺.

In analogy to the procedure described for Intermediate 52A, the following intermediate was prepared using the corresponding nitro starting material:

| Int. | Structure | Name | Analytical Data |
|---|---|---|---|
| 53A | 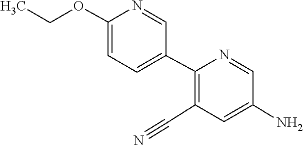 [SMILES: CCOc1ccc(-c2ncc(N)cc2C#N)cn1] | 5-amino-6′- ethoxy-2,3′- bipyridine-3- carbonitrile | LC (method 3): Rt = 0.91 min, MS (ESIpos) m/z = 241 (M + H)⁺. |

### Intermediate 54A: 3′,4′-dimethoxy-2-(1H-tetrazol-5-yl)biphenyl-4-amine

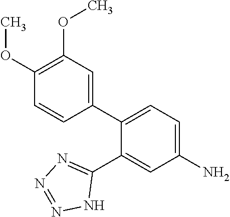 [SMILES: COc1ccc(-c2ccc(N)cc2-c2nnn[nH]2)cc1OC]

4-Amino-3′,4′-dimethoxybiphenyl-2-carbonitrile (12.9 g, 50.7 mmol) was dissolved in toluene (400 mL) and azidotrimethylsilane (26.9 mL, 203 mmol) and di-n-butyl tin oxide (18.9 g, 76.1 mmol) were added at RT. The resulting dark brown mixture was heated to 130° C. (bath temperature) for 14 h. The mixture was cooled and diluted with 250 ml methanol and 100 ml water. The mixture was extracted 3 times with ethyl acetate, the combined organic layers washed with water, brine, dried (Na₂SO₄) and filtered. Purification by chromatography (SiO₂, DCM/MeOH 5-22%) gave the title compound (13.6 g, 90% yield) as a light brown foam.

¹H NMR (400 MHz, DMSO-d6) δ[ppm] 3.55 (s, 3H), 3.71 (s, 3H), 6.47 (d, 1H), 6.75 (d, 1H), 6.81-6.83 (m, 2H), 7.24 (d, 1H).

LCMS (method 1): Rt=0.62 min, MS (ESipos) m/z=298 (M+H)⁺.

In analogy to the procedure described for Intermediate 54A, the following intermediates were prepared using the corresponding nitriles as starting materials.

| Int. | Structure | Name | Analytical Data |
|---|---|---|---|
| 55A | 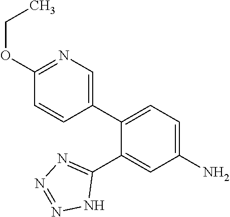 [SMILES: CCOc1ccc(-c2ccc(N)cc2-c2nnn[nH]2)cn1] | 4-(6- ethoxypyridin- 3-yl)-3-(1H- tetrazol-5- yl)aniline | ¹H NMR (400 MHz, DMSO-d6) δ [ppm] 1.30 (t, 3H), 4.26 (q, 2H), 6.64 (d, 1H), 6.81-6.84 (m, 2H), 7.19-7.22 (m, 2H), 7.80 (d, 1H). LCMS (method 1): Rt = 0.68 min, MS (ESIpos) m/z = 283 (M + H)⁺. |
| 56A | 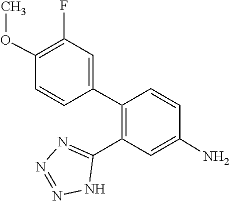 [SMILES: COc1ccc(-c2ccc(N)cc2-c2nnn[nH]2)cc1F] | 3′-fluoro-4′- methoxy-2-(1H- tetrazol-5- yl)biphenyl-4- amine | ¹H NMR (400 MHz, DMSO-d6) δ [ppm] 3.80 (s, 3H), 6.62-6.65 (m, 1H), 6.77 (d, 1H), 6.80-6.86 (m, 2H), 7.01 (t, 1H), 7.20 (d, 1H). LCMS (method 1): Rₜ = 0.72 min, MS (ESIpos) m/z = 286 (M + H)⁺. |
| 57A | 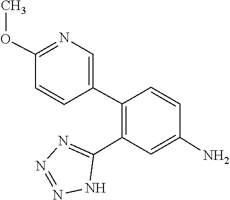 [SMILES: COc1ccc(-c2ccc(N)cc2-c2nnn[nH]2)cn1] | 4-(6- methoxypyridin- 3-yl)-3-(1H- tetrazol-5- yl)aniline | ¹H NMR (500 MHz, Methanol-d4) δ [ppm] 7.81 (d, J = 2.5 Hz, 1H), 7.31 (dd, J = 8.6, 2.5 Hz, 1H), 7.22 (d, J = 8.3 Hz, 1H), 6.96 (d, J = 2.4 Hz, 1H), 6.91 (dd, J = 8.3, 2.5 Hz, 1H), 6.65 (dd, J = 8.6, 0.6 Hz, 1H), 3.86 (s, 3H). LCMS (Analytical Method A): Rₜ = 0.81 min, MS (ESIpos) m/z = 269 (M + H)⁺. |
| 58A | 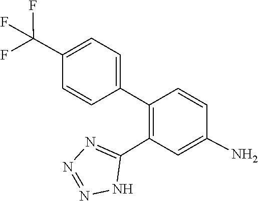 [SMILES: Nc1ccc(-c2ccc(C(F)(F)F)cc2)c(-c2nnn[nH]2)c1] | 2-(1H-tetrazol- 5-yl)-4′- (trifluoromethyl) biphenyl-4- amine | ¹H NMR (400 MHz, DMSO-d6) δ [ppm] 6.84-6.87 (m, 2H), 7.16 (d, 2H), 7.27 (d, 1H), 7.59 (d, 2H). LCMS (method 1): Rₜ = 0.93 min, MS (ESIpos) m/z = 306 (M + H)⁺. |
| 59A | 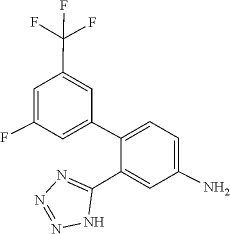 [SMILES: Nc1ccc(-c2cc(F)cc(C(F)(F)F)c2)c(-c2nnn[nH]2)c1] | 3′-fluoro-2-(1H- tetrazol-5-yl)- 5′- (trifluoromethyl) biphenyl-4- amine | ¹H NMR (400 MHz, DMSO-d6) δ = 5.77 (s, br, 2H), 6.85-6.87 (m, 2H), 7.01 (s, 1H), 7.19 (d, 1H), 7.33 (d, 1H), 7.52 (d, 1H). LCMS (method 2): Rt = 0.96 min, MS (ESIpos) m/z = 324 (M + H)⁺ |
| 60A | 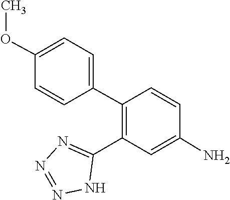 [SMILES: COc1ccc(-c2ccc(N)cc2-c2nnn[nH]2)cc1] | 4′-methoxy-2- (1H-tetrazol-5- yl)biphenyl-4- amine | ¹H NMR (500 MHz, DMSO-d6) δ [ppm] 7.17 (d, J = 8.3 Hz, 1H), 6.89-6.85 (m, 2H), 6.83-6.77 (m, 3H), 6.75 (d, J = 2.4 Hz, 1H), 5.47 (s, 2H), 3.71 (s, 3H). LCMS (Analytical Method A): Rt = 0.89 mins, MS (ESIpos) m/z = 268 (M + H)⁺. |
| 61A | 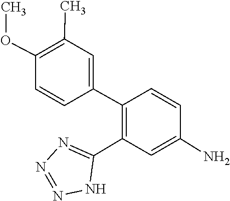 [SMILES: COc1ccc(-c2ccc(N)cc2-c2nnn[nH]2)cc1C] | 4′-methoxy-3′- methyl-2-(1H- tetrazol-5- yl)biphenyl-4- amine | ¹H NMR (500 MHz, DMSO-d6) δ [ppm] 2.05 (s, 3H), 3.73 (s, 3H), 5.44 (s, 2H), 6.66 (dd, J = 8.4, 2.3 Hz, 1H), 6.72-6.83 (m, 4H), 7.16 (d, J = 8.3 Hz, 1H). LCMS (Analytical Method A): Rt = 0.99 mins, MS (ESIpos) m/z = 282 (M + H)⁺. |
| 62A | 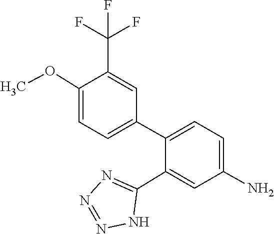 [SMILES: COc1ccc(-c2ccc(N)cc2-c2nnn[nH]2)cc1C(F)(F)F] | 4′-methoxy-2- (1H-tetrazol-5- yl)-3′- (trifluoromethyl) biphenyl-4- amine | ¹H NMR (400 MHz, DMSO-d6) δ = 3.86 (s, 3H), 5.60 (s, br, 2H), 6.80 (d, 1H), 6.84 (dd, 1H), 7.12- 7.19 (m, 3H), 7.24 (d, 1H). LCMS (method 2): Rt = 0.91 min, m/z = 336 (M + H)⁺ |
| 63A | 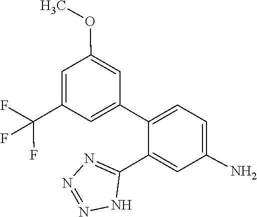 [SMILES: COc1cc(-c2ccc(N)cc2-c2nnn[nH]2)cc(C(F)(F)F)c1] | 3′-methoxy-2- (1H-tetrazol-5-yl)-5′- (trifluoromethyl) biphenyl-4- amine | ¹H NMR (400 MHz, DMSO-d6) δ = 3.73 (s, 3H), 5.70 (s, br, 2H), 6.76 (s, 1H), 6.78 (d, 1H), 6.81 (s, 1H), 6.85 (d, 1H), 6.87 (d, 1H), 7.07 (s, 1H), 7.32 (d, 1H). LCMS (method 2): Rt = 0.95 min, MS (ESIpos) m/z = 336 (M + H)⁺ |
| 64A | 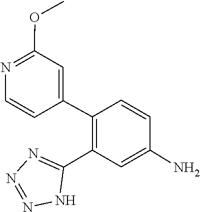 [SMILES: COc1cc(-c2ccc(N)cc2-c2nnn[nH]2)ccn1] | 4-(2- methoxypyridin- 4-yl)-3-(1H- tetrazol-5- yl)aniline | ¹H NMR (500 MHz, DMSO-d6) δ [ppm] 7.95 (d, J = 5.3 Hz, 1H), 7.29 (d, J = 8.4 Hz, 1H), 6.88- 6.78 (m, 2H), 6.46 (m, 1H), 6.38 (s, 1H), 3.80 (s, 3H). LCMS (Analytical Method A): Rₜ = 0.81 min; MS (ESIpos) m/z 269 (M + H)⁺. |
| 65A | 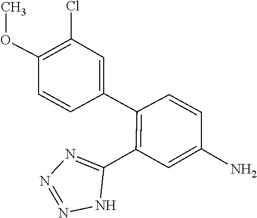 [SMILES: COc1ccc(-c2ccc(N)cc2-c2nnn[nH]2)cc1Cl] | 3′-chloro-4′- methoxy-2-(1H- tetrazol-5- yl)biphenyl-4- amine | ¹H NMR (250 MHz, Methanol-d4) δ [ppm] 7.26 (m, 1H), 7.10 (d, J = 2.1 Hz, 1H), 6.99-6.91 (m, 3H), 6.86 (m, 1H), 3.86 (s, 3H). LCMS (Analytical Method A): Rₜ = 0.97 min; MS (ESIpos) m/z 302 (M + H)⁺. |
| 66A | 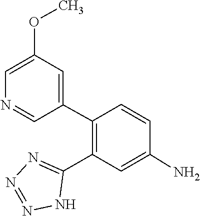 [SMILES: COc1cncc(-c2ccc(N)cc2-c2nnn[nH]2)c1] | 4-(5- methoxypyridin- 3-yl)-3-(1H- tetrazol-5- yl)aniline | ¹H NMR (500 MHz, DMSO-d6) δ [ppm] 8.12 (d, J = 2.8 Hz, 1H), 7.76 (d, J = 1.8 Hz, 1H), 7.30 (d, J = 8.3 Hz, 1H), 6.92 (m, 1H), 6.89-6.80 (m, 2H), 5.67 (s, 2H), 3.72 (s, 3H). |
| 67A | 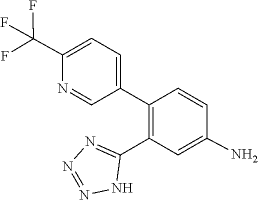 [SMILES: Nc1ccc(-c2ccc(C(F)(F)F)nc2)c(-c2nnn[nH]2)c1] | 3-(1H-tetrazol- 5-yl)-4-[6- (trifluoromethyl) pyridin-3- yl]aniline | ¹H NMR (500 MHz, DMSO-d6) δ [ppm] 5.79 (s, 2H), 6.88 (m, 1H), 6.92 (s, 1H), 7.33 (d, J = 8.4 Hz, 1H), 7.60 (m, 1H), 7.77 (d, J = 8.1 Hz, 1H), 8.40 (d, J = 2.1 Hz, 1H). LCMS (Analytical Method A): Rₜ = 0.97 min; MS (ESIpos) m/z 307 (M + H)⁺. |
| 68A | 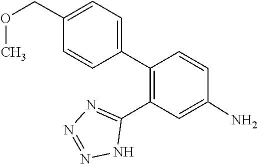 [SMILES: COCc1ccc(-c2ccc(N)cc2-c2nnn[nH]2)cc1] | 4′- (methoxymethyl)- 2-(1H- tetrazol-5- yl)biphenyl-4- amine | ¹H NMR (250 MHz, Methanol-d4) δ [ppm] 7.32-7.20 (m, 3H), 7.06- 6.92 (m, 4H), 4.43 (s, 2H), 3.37 (s, 3H). LCMS (Analytical Method A): Rₜ = 0.89 min; MS (ESIpos) m/z 282 (M + H)⁺. |
| 69A | 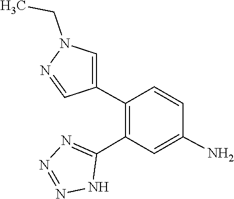 [SMILES: CCn1cc(-c2ccc(N)cc2-c2nnn[nH]2)cn1] | 4-(1-ethyl-1H- pyrazol-4-yl)-3- (1H-tetrazol-5- yl)aniline | ¹H NMR (400 MHz, DMSO-d6) δ = 1.29 (t, 3H), 4.02 (q, 2H), 6.66 1H), (d, 1H), 6.78 (dd, 1H), 6.91 (d, 7.25 (d, 1H), 7.33 (d, 1H). LCMS (method 1): Rt = 0.44 min, m/z = 256 (M + H)⁺. |
| 70A | 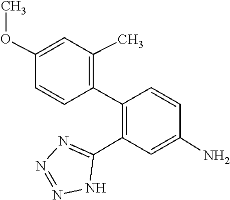 [SMILES: COc1ccc(-c2ccc(N)cc2-c2nnn[nH]2)c(C)c1] | 4′-methoxy-2′- methyl-2-(1H- tetrazol-5- yl)biphenyl-4- amine | ¹H NMR (500 MHz, DMSO-d6) δ [ppm] 1.89 (s, 3H), 3.70 (s, 3H), 5.42 (s, 2H), 6.62 (m, 1H), 6.68 (d, J = 2.6 Hz, 1H), 6.76-6.85 (m, 3H), 6.97 (d, J = 8.2 Hz, 1H). LCMS (Analytical Method A): Rt = 0.94 min; MS (ESIpos) m/z = 282 (M + H)⁺. |
| 71A | 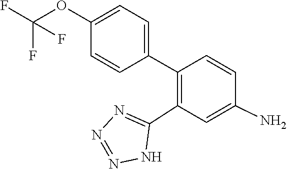 [SMILES: Nc1ccc(-c2ccc(OC(F)(F)F)cc2)c(-c2nnn[nH]2)c1] | 2-(1H-tetrazol- 5-yl)-4′- (trifluoromethoxy) biphenyl-4- amine | ¹H NMR (500 MHz, Methanol-d4) δ [ppm] 6.91-7.00 (m, 2H), 7.07- 7.13 (m, 2H), 7.15 (d, J = 8.3 Hz, 2H), 7.28 (d, J = 8.2 Hz, 1H). LCMS (Analytical Method A): Rt = 1.05 min; MS (ESIpos) m/z = 322 (M + H)⁺. |
| 72A | 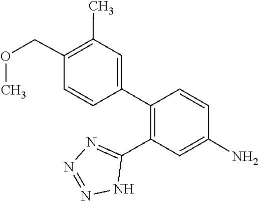 [SMILES: COCc1ccc(-c2ccc(N)cc2-c2nnn[nH]2)cc1C] | 4′- (methoxymethyl)- 3′-methyl-2- (1H-tetrazol-5- yl)biphenyl-4- amine | ¹H NMR (250 MHz, DMSO-d6) δ [ppm] 2.17 (s, 3H), 3.29 (s, 3H), 4.34 (s, 2H), 5.51 (s, 2H), 6.65 (m, 1H), 6.71-6.91 (m, 3H), 7.09 (d, J = 7.8 Hz, 1H), 7.20 (d, J = 8.3 Hz, 1H). LCMS (Analytical Method A): Rₜ = 0.94 min; MS (ESIpos) m/z = 296 (M + H)⁺. |
| 73A | 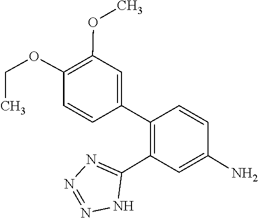 [SMILES: CCOc1ccc(-c2ccc(N)cc2-c2nnn[nH]2)cc1OC] | 4′-ethoxy-3′- methoxy-2-(1H- tetrazol-5- yl)biphenyl-4- amine | ¹H NMR (500 MHz, DMSO-d6) δ [ppm] 1.29 (t, J = 6.9 Hz, 3H), 3.55 (s, 3H), 3.95 (q, J = 6.8 Hz, 2H), 5.75 (s, 2H), 6.38-6.51 (m, 2H), 6.69-6.88 (m, 3H), 7.23 (d, J = 8.3 Hz, 1H). LCMS (Analytical Method A): Rₜ = 1.12 min; MS (ESIpos) m/z = 312 (M + H)⁺. |
| 74A | 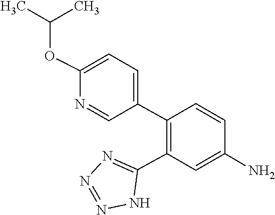 [SMILES: CC(C)Oc1ccc(-c2ccc(N)cc2-c2nnn[nH]2)cn1] | 4-(6- isopropoxypyridin- 3-yl)-3-(1H- tetrazol-5- yl)aniline | ¹H NMR (500 MHz, DMSO-d6) δ [ppm] 1.26 (d, J = 6.2 Hz, 6H), 5.19 (hept, J = 6.2 Hz, 1H), 5.57 (s, 2H), 6.59 (d, J = 8.5 Hz, 1H), 6.77-6.86 (m, 2H), 7.11-7.25 (m, 2H), 7.79 (d, J = 2.5 Hz, 1H). LCMS (Analytical Method A): Rₜ = 0.94 min; MS (ESIpos) m/z = 297 (M + H)⁺. |
| 75A | 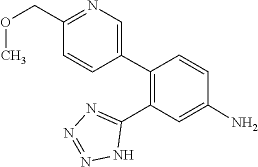 [SMILES: COCc1ccc(-c2ccc(N)cc2-c2nnn[nH]2)cn1] | 4-[6- (methoxymethyl) pyridin-3-yl]-3- (1H-tetrazol-5- yl)aniline | ¹H NMR (500 MHz, DMSO-d6) δ [ppm] 3.35 (s, 3H), 4.45 (s, 2H), 5.64 (s, 2H), 6.81-6.86 (m, 2H), 7.21-7.28 (m, 2H), 7.30-7.36 (m, 1H), 8.13 (d, J = 2.2 Hz, 1H). LCMS (Analytical Method C): Rₜ = 0.84 min; MS (ESIpos) m/z = 283 (M + H)⁺. |
| 76A | 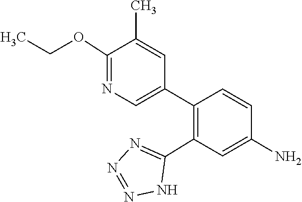 [SMILES: CCOc1ncc(-c2ccc(N)cc2-c2nnn[nH]2)cc1C] | 4-(6-ethoxy-5- methylpyridin- 3-yl)-3-(1H- tetrazol-5- yl)aniline | ¹H NMR (500 MHz, DMSO-d6) δ [ppm] 7.56-7.52 (m, 1H), 7.20- 7.15 (m, 1H), 7.12 (dd, J = 2.4, 0.8 Hz, 1H), 6.84-6.82 (m, 1H), 6.82-6.81 (m, 1H), 5.65 (s, 2H), 4.28 (q, J = 7.0 Hz, 2H), 2.04 (s, 3H), 1.30 (t, J = 7.0 Hz, 3H). LCMS (Analytical Method A): Rt = 1.00 mins; MS (ESIpos) m/z = 297 (M + H)⁺. |
| 77A | 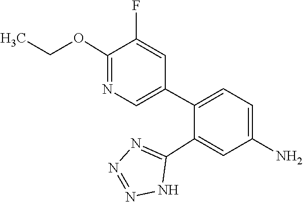 [SMILES: CCOc1ncc(-c2ccc(N)cc2-c2nnn[nH]2)cc1F] | 4-(6-ethoxy-5- fluoropyridin-3- yl)-3-(1H- tetrazol-5- yl)aniline | ¹H NMR (500 MHz, DMSO-d6) δ [ppm] 7.54 (d, J = 2.0 Hz, 1H), 7.28 (dd, J = 11.6, 2.0 Hz, 1H), 7.22 (d, J = 8.3 Hz, 1H), 6.86 (d, J = 2.3 Hz, 1H), 6.83 (dd, J = 8.3, 2.4 Hz, 1H), 4.36 (q, J = 7.0 Hz, 2H), 1.33 (t, J = 7.0 Hz, 3H). LCMS (Analytical Method A): Rt = 1.00 mins; MS (ESIpos) m/z = 301 (M + H)⁺. |
| 78A | 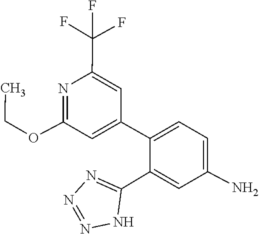 [SMILES: CCOc1cc(-c2ccc(N)cc2-c2nnn[nH]2)cc(C(F)(F)F)n1] | 4-[2-ethoxy-6- (trifluoromethyl) pyridin-4-yl]-3- (1H-tetrazol-5- yl)aniline | 1H NMR (400 MHz, DMSO-d6) δ = 1.31 (t, 3H), 4.29 (q, 2H), 6.66 (s, 1H), 6.81-6.86 (m, 3H), 7.36 (d, 1H). LCMS (method 1): Rt = 1.05 min, MS (ESIpos) m/z = 351 (M + H)+ |
| 79A | 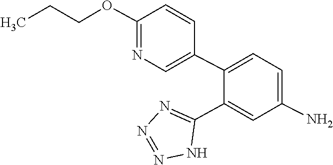 [SMILES: CCCOc1ccc(-c2ccc(N)cc2-c2nnn[nH]2)cn1] | 4-(6- propoxypyridin- 3-yl)-3-(1H- tetrazol-5- yl)aniline | ¹H NMR (500 MHz, Methanol-d4) δ [ppm] 7.79 (dd, J = 2.5, 0.6 Hz, 1H), 7.31 (dd, J = 8.6, 2.6 Hz, 1H), 7.28-7.23 (m, 1H), 6.98- 6.94 (m, 2H), 6.69 (dd, J = 8.6, 0.6 Hz, 1H), 4.18 (t, J = 6.6 Hz, 2H), 1.77 (m, 2H), 1.02 (t, J = 7.4 Hz, 3H). LCMS (Analytical Method A): Rt = 0.98 mins; MS (ESIpos) m/z = 297 (M + H)⁺. |
| 80A | 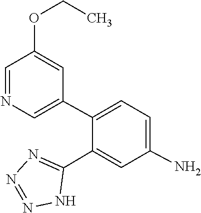 [SMILES: CCOc1cncc(-c2ccc(N)cc2-c2nnn[nH]2)c1] | 4-(5- ethoxypyridin-3- yl)-3-(1H- tetrazol-5- yl)aniline | ¹H NMR (500 MHz, DMSO-d6) δ [ppm] 8.09 (d, J = 2.7 Hz, 1H), 7.75 (d, J = 1.7 Hz, 1H), 7.28 (d, J = 8.2 Hz, 1H), 6.90-6.87 (m, 1H), 6.87-6.82 (m, 2H), 3.96 (q, J = 7.0 Hz, 2H), 1.28 (t, J = 7.0 Hz, 3H). LCMS (Analytical Method A): Rt = 0.98 mins; MS (ESIpos) m/z = 282 (M + H)⁺. |
| 81A | 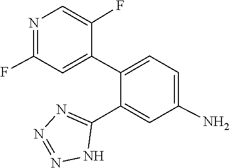 [SMILES: Nc1ccc(-c2cc(F)ncc2F)c(-c2nnn[nH]2)c1] | 4-(2,5- difluoropyridin- 4-yl)-3-(1H- tetrazol-5- yl)aniline | LCMS (method 3): Rt = 0.70 min, MS (ESIpos) m/z = 275 (M + H)⁺ |
| 82A | 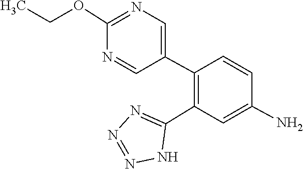 | 4-(2- ethoxypyrimidin- 5-yl)-3-(1H- tetrazol-5- yl)aniline | ¹H NMR (500 MHz, DMSO-d6) δ [ppm] 8.47-8.43 (m, 2H), 6.96 (d, J = 8.3 Hz, 1H), 6.76-6.69 (m, 2H), 3.77 (t, 2H), 3.61 (q, 3H). LCMS (Analytical Method A): Rt = 0.88 mins; MS (ESIpos) m/z = 294 (M + H)⁺. |
| 83A | 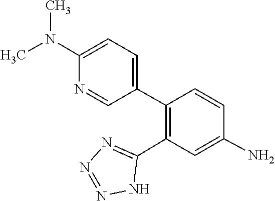 [SMILES: CN(C)c1ccc(-c2ccc(N)cc2-c2nnn[nH]2)cn1] | 5-[4-amino-2- (1H-tetrazol-5- yl)phenyl]-N,N- dimethylpyridin- 2-amine | ¹H NMR (500 MHz, DMSO-d6) δ [ppm] 7.75 (d, J = 2.3 Hz, 1H), 7.16 (d, J = 8.3 Hz, 1H), 7.02 (dd, J = 8.8, 2.5 Hz, 1H), 6.82 (dd, J = 8.3, 2.3 Hz, 1H), 6.78 (d, J = 2.3 Hz, 1H), 6.49 (d, J = 8.8 Hz, 1H), 5.66 (br. s, 2H), 2.98 (s, 6H). LCMS (Analytical Method F): Rt = 2.03 mins; MS (ESIpos) m/z = 282 (M + H)⁺. |
| 84A | 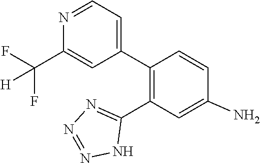 [SMILES: Nc1ccc(-c2ccnc(C(F)F)c2)c(-c2nnn[nH]2)c1] | 4-[2- (difluoromethyl) pyridin-4-yl]-3- (1H-tetrazol-5- yl)aniline | 1H NMR (500 MHz, DMSO-d6) δ [ppm] 8.48 (d, J = 5.1 Hz, 1H), 7.35 (d, J = 9.0 Hz, 1H), 7.19 (s, 1H), 7.09 (d, J = 5.1 Hz, 1H), 7.00-6.70 (m, 3H), 5.83 (s, 2H). LCMS (Analytical Method F): Rt = 1.65 mins, MS (ESIpos) m/z = 289 (M + H)⁺. |
| 85A | 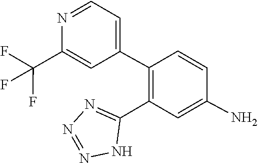 [SMILES: Nc1ccc(-c2ccnc(C(F)(F)F)c2)c(-c2nnn[nH]2)c1] | 3-(1H-tetrazol- 5-yl)-4-[2- (trifluoromethyl) pyridin-4- yl]aniline | ¹H NMR (500 MHz, DMSO-d6) δ [ppm] 8.57 (d, J = 5.1 Hz, 1H), 7.40 (d, J = 9.1 Hz, 1H), 7.36- 7.33 (m, 1H), 7.23 (dd, J = 5.1, 1.3 Hz, 1H), 6.92-6.85 (m, 2H), 5.91 (s, 2H). LCMS (Analytical Method F): Rt = 2.08 mins; MS (ESIpos) m/z = 307 (M + H)⁺. |
| 86A | 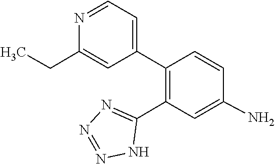 [SMILES: CCc1cc(-c2ccc(N)cc2-c2nnn[nH]2)ccn1] | 4-(2- ethylpyridin-4- yl)-3-(1H- tetrazol-5- yl)aniline | ¹H NMR (400 MHz, DMSO-d6) δ = 1.11 (t, 3H), 2.61 (q, 2H), 7.27- 7.31 (m, 1H), 6.73 (dd, 1H), 6.77- 6.81 (m, 3H), 7.25 (d, 1H), 8.24 (d, 1H). LCMS (method 1): Rt = 0.31 min, MS (ESIpos) m/z = 267 (M + H)⁺. |
| 87A | 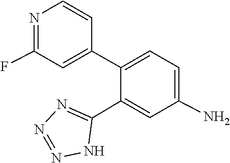 [SMILES: Nc1ccc(-c2ccnc(F)c2)c(-c2nnn[nH]2)c1] | 4-(2- fluoropyridin-4- yl)-3-(1H- tetrazol-5- yl)aniline | ¹H NMR (400 MHz, DMSO-d6) δ = 6.77-6.79 (m, 2H), 6.84-6.87 (m, 2H), 7.35 (d, 1H), 8.04 (d, 1H). LCMS (method 1): Rt = 0.53 min, MS (ESIpos) m/z = 257 (M + H)⁺ |
| 88A | 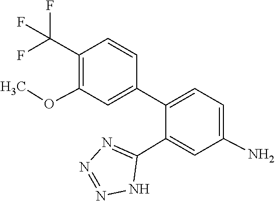 [SMILES: COc1cc(-c2ccc(N)cc2-c2nnn[nH]2)ccc1C(F)(F)F] | 3′-methoxy-2- (1H-tetrazol-5- yl)-4′- (trifluoromethyl) biphenyl-4- amine | ¹H NMR (400 MHz, DMSO-d6) δ = 3.62 (s, 3H), 6.69-6.72 (m, 3H), 6.78 (d, 1H), 7.21 (d, 1H), 7.37 (d, 1H). LCMS (method 1): Rt = 0.96 min, MS (ESIpos) m/z = 336 (M + H)⁺ |
| 89A | 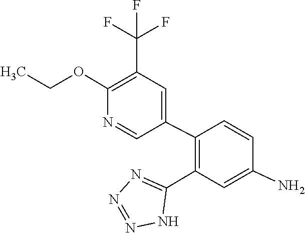 [SMILES: CCOc1ncc(-c2ccc(N)cc2-c2nnn[nH]2)cc1C(F)(F)F] | 4-[6-ethoxy-5- (trifluoromethyl) pyridin-3-yl]-3- (1H-tetrazo-5- yl)aniline | ¹H NMR (500 MHz, DMSO-d6) δ [ppm] 8.03 (d, J = 2.2 Hz, 1H), 7.57 (d, J = 2.2 Hz, 1H), 7.28 (d, J = 8.3 Hz, 1H), 6.90 (d, J = 1.8 Hz, 1H), 6.85 (dd, J = 8.3, 2.3 Hz, 1H), 5.72 (s, 2H), 4.42 (q, J = 7.0 Hz, 2H), 1.32 (t, J = 7.0 Hz, 3H). LCMS (Analytical Method F): Rt = 2.91 mins; MS (ESIpos) m/z = 351 (M + H)⁺. |
| 90A | 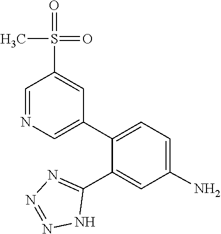 [SMILES: CS(=O)(=O)c1cncc(-c2ccc(N)cc2-c2nnn[nH]2)c1] | 4-[5- (methylsulfonyl) pyridin-3-yl]-3- (1H-tetrazol-5- yl)aniline | ¹H NMR (400 MHz, DMSO-d6) δ [ppm] 3.17 (s, 3H), 6.75 (dd, 1H), 6.89 (d, 1H), 7.26 (d, 1H), 7.82 (dd, 1H), 8.49 (d, 1H), 8.81 (d, 1H). LCMS (method 1): Rₜ = 0.42 min, MS (ESIpos) m/z = 317 (M + H)⁺ |
| 91A | 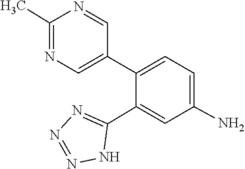 [SMILES: Cc1ncc(-c2ccc(N)cc2-c2nnn[nH]2)cn1] | 4-(2- methylpyrimidin 5-yl)-3-(1H- tetrazol-5- yl)aniline | ¹H NMR (500 MHz, DMSO-d6) δ [ppm] 8.29 (s, 2H), 7.23 (d, J = 8.3 Hz, 1H), 6.94 (d, J = 2.0 Hz, 1H), 6.83 (dd, J = 8.3, 1.9 Hz, 1H), 2.58 (s, 3H). LCMS (Analytical Method F): Rt = 1.06 mins; MS (ESIpos) m/z = 254 (M + H)⁺. |
| 92A | 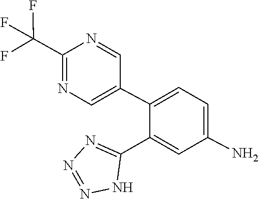 [SMILES: Nc1ccc(-c2cnc(C(F)(F)F)nc2)c(-c2nnn[nH]2)c1] | 3-(1H-tetrazol- 5-yl)-4-[2- (trifluoromethyl) pyrimidin-5- yl]aniline | ¹H NMR (500 MHz, DMSO-d6) δ [ppm] 8.72 (s, 2H), 7.39 (d, J = 8.4 Hz, 1H), 7.03 (d, 1H), 6.90 (dd, J = 8.4, 2.3 Hz, 1H), 5.92 (s, 2H). LCMS (Analytical Method D): Rt = 3.31 mins; MS (ESIpos) m/z = 308 (M + H)⁺. |
| 93A | 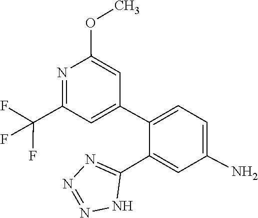 [SMILES: COc1cc(-c2ccc(N)cc2-c2nnn[nH]2)cc(C(F)(F)F)n1] | 4-[2-methoxy-6- (trifluoromethyl) pyridin-4-yl]-3- (1H-tetrazol-5- yl)aniline | ¹H NMR (500 MHz, DMSO-d6) δ [ppm] 7.32 (d, J = 8.4 Hz, 1H), 6.90-6.84 (m, 2H), 6.80 (dd, J = 8.4, 2.3 Hz, 1H), 6.67 (s, 1H), 3.85 (s, 3H). LCMS (Analytical Method F): Rt = 2.73 mins; MS (ESIpos) m/z = 337 (M + H)⁺. |
| 94A | 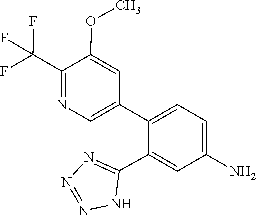 [SMILES: COc1cc(-c2ccc(N)cc2-c2nnn[nH]2)cnc1C(F)(F)F] | 4-[5-methoxy-6- (trifluoromethyl) pyridin-3-yl]-3- (1H-tetrazol-5- yl)aniline | ¹H NMR (400 MHz, DMSO-d6) δ [ppm] 3.80 (s, 3H), 5.84 (s, br, 2H), 6.88-6.92 (m, 2H), 7.34 (s, 1H), 7.40 (d, 1H), 7.80 (d, 1H). LCMS (method 2): Rt = 0.81 min, MS (ESIpos) m/z = 337 (M + H)⁺ |
| 95A | 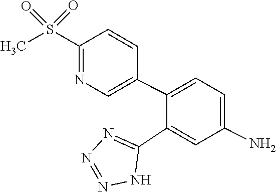 [SMILES: CS(=O)(=O)c1ccc(-c2ccc(N)cc2-c2nnn[nH]2)cn1] | 4-[6- (methylsulfonyl) pyridin-3-yl]-3- (1H-tetrazol-5- yl)aniline | ¹H NMR (500 MHz, DMSO-d6) δ [ppm] 8.42 (d, J = 1.8 Hz, 1H), 7.90 (d, J = 8.2 Hz, 1H), 7.65 (dd, J = 8.1, 2.0 Hz, 1H), 7.33 (d, J = 8.4 Hz, 1H), 6.93 (d, 1H), 6.88 (dd, J = 8.4, 2.2 Hz, 1H), 3.27 (s, 3H). LCMS (Analytical Method F): Rt = 1.32 min; MS (ESIpos) m/z = 317 (M + H)⁺. |
| 96A | 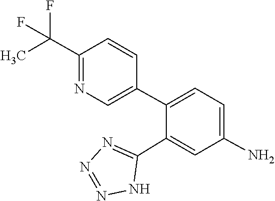 [SMILES: CC(F)(F)c1ccc(-c2ccc(N)cc2-c2nnn[nH]2)cn1] | 4-[6-(1,1- difluoroethyl) pyridin-3-yl]-3- (1H-tetrazol-5- yl)aniline | LCMS (Analytical Method A): Rt = 0.96 mins; MS (ESIpos) m/z = 303 (M + H)⁺. |
| 97A | 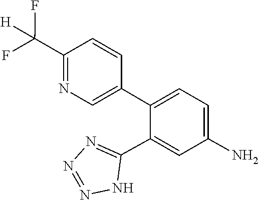 [SMILES: Nc1ccc(-c2ccc(C(F)F)nc2)c(-c2nnn[nH]2)c1] | 4-[6- (difluoromethyl) pyridin-3-yl]-3- (1H-tetrazol-5- yl)aniline | ¹H NMR (500 MHz, DMSO-d6) δ [ppm] 8.30 (d, J = 1.9 Hz, 1H), 7.56 (d, J = 8.1 Hz, 1H), 7.52 (dd, J = 8.1, 2.1 Hz, 1H), 7.30 (d, J = 8.3 Hz, 1H), 7.06-6.79 (m, 3H), 5.76 (br s, 2H). LCMS (Analytical Method F): Rt = 1.78 mins; MS (ESIpos) m/z = 289.1 (M + H)⁺. |
| 98A | 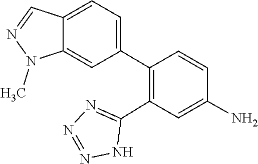 [SMILES: Cn1ncc2ccc(-c3ccc(N)cc3-c3nnn[nH]3)cc21] | 4-(1-methyl-1H- indazol-6-yl)-3- (1H-tetrazol-5- yl)aniline | ¹H NMR (500 MHz, DMSO-d6) δ [ppm] 7.95 (s, 1H), 7.50 (d, J = 8.3 Hz, 1H), 7.35-7.26 (m, 2H), 6.89-6.80 (m, 2H), 6.57 (d, J = 7.6 Hz, 1H), 5.54 (s, 2H), 3.96 (s, 3H). LCMS (Analytical Method F): Rt = 1.73 mins; MS (ESIpos) m/z = 292 (M + H)⁺. |
| 99A | 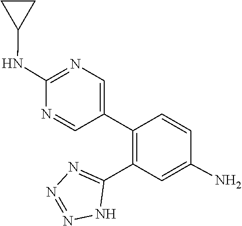 [SMILES: Nc1ccc(-c2cnc(NC3CC3)nc2)c(-c2nnn[nH]2)c1] | 5-[4-amino-2- (1H-tetrazol-5- yl)phenyl]-N- cyclopropylpyrimidin- 2-amine | ¹H NMR (400 MHz, DMSO-d6) δ [ppm] 0.43-0.47 (m, 2H), 0.62- 0.66 (m, 2H), 2.64-2.68 (m, 1H), 6.77 (dd, 1H), 6.87 (d, 1H), 7.13 (d, 1H), 7.29 (d, 1H), 7.91 (s, 2H). LCMS (method 1): Rₜ = 0.45 min, MS (ESIpos) m/z = 295 (M + H)⁺. |
| 100A | 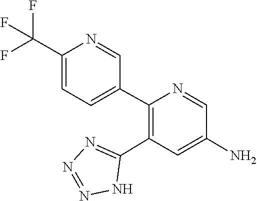 [SMILES: Nc1cnc(-c2ccc(C(F)(F)F)nc2)c(-c2nnn[nH]2)c1] | 3-(1H-tetrazol- 5-yl)-6′- (trifluoromethyl)- 2,3′-bipyridin- 5-amine | ¹H NMR (400 MHz, DMSO-d6) δ [ppm] 7.28 (d, 1H), 7.75 (dd, 1H), 7.80 (dd, 1H), 8.23 (d, 1H), 8.53 (d, 1H). LCMS (method 3): Rt = 0.69 min, MS (ESIpos) m/z = 308 (M + H)⁺. |
| 101A | 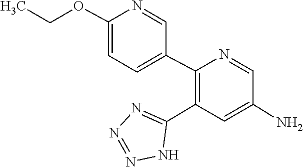 [SMILES: CCOc1ccc(-c2ncc(N)cc2-c2nnn[nH]2)cn1] | 6′-ethoxy-3-(1H- tetrazol-5-yl)- 2,3′-bipyridin-5- amine | ¹H NMR (400 MHz, DMSO-d6) δ [ppm] 1.29 (t, 3H), 4.27 (q, 2H), 5.83 (s, br, 2H), 6.69 (dd, 1H), 7.17 (d, 1H), 7.41 (dd, 1H), 7.85 (d, 1H), 8.19 (d, 1H). LCMS (method 3): Rt = 0.57 min, MS (ESIpos) m/z = 284 (M + H)⁺ |
| 102A | 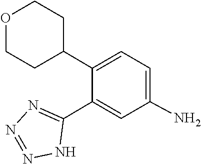 [SMILES: Nc1ccc(C2CCOCC2)c(-c2nnn[nH]2)c1] | 4-(tetrahydro- 2H-pyran-4-yl)- 3-(1H-tetrazol- 5-yl)aniline | ¹H NMR (250 MHz, DMSO-d6) δ [ppm] 1.37-1.75 (m, 4H), 2.81- 3.01 (m, 1H), 3.19-3.26 (m, 2H), 3.76-3.92 (m, 2H), 4.08 (s, 2H), 6.66 (d, J = 2.4 Hz, 1H), 6.76 (dd, J = 8.4, 2.5 Hz, 1H), 7.18 (d, J = 8.5 Hz, 1H). LCMS (Analytical Method A): Rt = 0.66 mins; MS (ESIpos) m/z = 246 (M + H)⁺. |
| 103A | 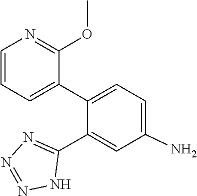 [SMILES: COc1ncccc1-c1ccc(N)cc1-c1nnn[nH]1] | 4-(2- methoxypyridin- 3-yl)-3-(1H- tetrazol-5- yl)aniline | ¹H NMR (400 MHz, DMSO-d6) δ [ppm] 3.40 (s, 3H), 6.81-6.86 (m, 2H), 7.00 (dd, 1H), 7.14 (d, 1H), 7.51 (dd, 1H), 8.04 (dd, 1H). LCMS (method 1): Rₜ = 0.54 min, MS (ESIpos) m/z = 269 (M + H)⁺. |
| 104A | 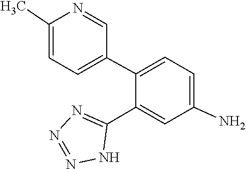 [SMILES: Cc1ccc(-c2ccc(N)cc2-c2nnn[nH]2)cn1] | 4-(6- methylpyridin- 3-yl)-3-(1H- tetrazol-5- yl)aniline | ¹H NMR (500 MHz, DMSO-d6) δ [ppm] 8.02-7.94 (m, 1H), 7.19- 7.08 (m, 2H), 7.08-6.99 (m, 1H), 6.80-6.71 (m, 2H), 5.98- 5.08 (br. s 2H), 2.35 (s, 3H). LCMS (Analytical Method E); Rt = 2.2 min, MS (ESIpos) m/z = 253 (M + H)⁺. |
| 105A | 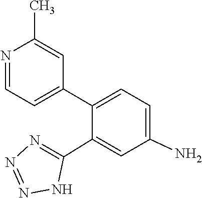 [SMILES: Cc1cc(-c2ccc(N)cc2-c2nnn[nH]2)ccn1] | 4-(2- methylpyridin- 4-yl)-3-(1H- tetrazol-5- yl)aniline | ¹H NMR (500 MHz, DMSO-d6) δ [ppm] 8.20 (d, J = 5.2 Hz, 1H), 7.28-7.20 (m, 1H), 6.87 (s, 1H), 6.84-6.77 (m, 2H), 6.68-6.60 (m, 1H), 5.66 (s, 2H), 2.36 (s, 3H). LCMS (Analytical Method E): Rt = 0.74 and 1.51 min, MS (ESIpos) m/z = 253 (M + H)⁺. |
| 107A | 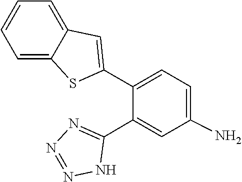 [SMILES: Nc1ccc(-c2cc3ccccc3s2)c(-c2nnn[nH]2)c1] | 4-(1- benzothiophen- 2-yl)-3-(1H- 1,2,3,4- tetrazol-5- yl)aniline | ¹H NMR (500 MHz, DMSO-d6) δ [ppm] 5.79 (s, 2H), 6.77 (d, J = 2.2 Hz, 1H), 6.86 (d, J = 8.5 Hz, 1H), 6.89 (s, 1H), 7.22-7.35 (m, 2H), 7.45 (d, J = 8.4 Hz, 1H), 7.69 (d, J = 8.0 Hz, 1H), 7.83 (d, J = 7.8 Hz, 1H). LCMS (Analytical Method D) Rt = 3.67 min, MS (ESIpos) m/z = 293.9 (M + H)⁺. |
| 108A | 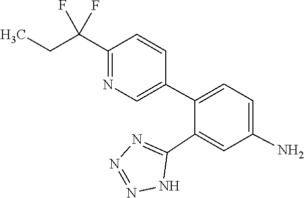 [SMILES: CCC(F)(F)c1ccc(-c2ccc(N)cc2-c2nnn[nH]2)cn1] | 4-[6-(1,1- difluoropropyl) pyridin-3-yl]-3- (1H-tetrazol-5- yl)aniline | ¹H NMR (500 MHz, DMSO-d6) δ 8.33-8.25 (m, 1H), 7.57-7.41 (m, 2H), 7.26 (d, J = 8.3 Hz, 1H), 6.89 (d, J = 2.3 Hz, 1H), 6.85- 6.80 (m, 1H), 2.35-2.23 (m, 2H), 0.91 (t, J = 7.5 Hz, 3H). LCMS (Analytical Method E) Rt = 2.36 min, MS (ESIpos) m/z = 317 (M + H)⁺. |

### Intermediate 109A: 4-bromo-3-(1H-tetrazol-5-yl)aniline

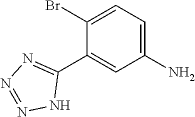 [SMILES: Nc1ccc(Br)c(-c2nnn[nH]2)c1]

5-Amino-2-bromobenzonitrile (3.90 g, 19.8 mmol) was dissolved in toluene (210 mL) and azidotrimethylsilane (10.1 mL, 79.2 mmol) and di-n-butyl tin oxide (7.39 g, 29.7 mmol) were added at RT. The mixture was heated to 130° C. (bath temperature) for 10 h. The mixture was cooled and diluted with methanol. Silica gel was added and the solvents evaporated under reduced pressure. The residue was purified by chromatography p (SiO₂, DCM/MeOH 0-10-30-50%) to give the title compound (4.50 g, 95% yield) as a yellow solid.

¹H NMR (400 MHz, DMSO-d6) δ[ppm] 6.70 (dd, 1H), 6.83 (d, 1H), 7.41 (d, 1H).

LCMS (method 1): Rt=0.47 min; MS (ESipos) m/z=240 (M+H)⁺.

### Intermediate 110A: 4N-[4-bromo-3-(1H-tetrazol-5-yl)phenyl]-1-(3-chlorophenyl)cyclopropanecarboxamide

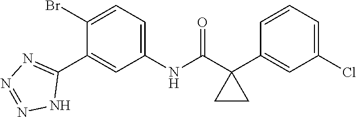 [SMILES: O=C(Nc1ccc(Br)c(-c2nnn[nH]2)c1)C1(c2cccc(Cl)c2)CC1]

a) 1-(3-Chlorophenyl)cyclopropanecarboxylic acid (15 g, 76.3 mmol; CAS 124276-34-2) and thionyl chloride (90.8 g, 763 mmol) were heated to reflux until gas evaporation ceased (1 h). The mixture was cooled to RT and the excess thionyl chloride was evaporated under reduced pressure. The crude acid chloride was used without further purification.

b) 4-Bromo-3-(1H-tetrazol-5-yl)aniline (Intermediate 109A; 500 mg, 2.08 mmol) was dissolved in pyridine (2 mL) and 1-(3-chlorophenyl)cyclopropanecarbonyl chloride (493 mg, 2.29 mmol; prepared in step a) were added dropwise at 0° C. The mixture was stirred at RT for 3 h. The diluted with water and extracted with ethyl acetate (×3). The combined organic layers were washed with brine, dried (Na₂SO₄), filtered and the solvents removed under reduced pressure. The residue was purified by flash chromatography (SiO₂, DCM/MeOH 0-10-20%) to give the title compound (855 mg, 90% yield) as a yellow solid.

¹H NMR (400 MHz, DMSO-d6) δ[ppm] 1.17-1.20 (m, 2H), 1.47-1.50 (m, 2H), 7.35-7.38 (m, 3H), 7.42-7.44 (m, 1H), 7.74-7.76 (m, 2H), 7.95 (d, 1H), 9.52 (s, 1H).

LCMS (method 1): Rt=1.12 min, MS (ESIpos) m/z=418/420/422 (M+H, ClBr isotope pattern)⁺.

### Intermediate 111A: N-[4-bromo-3-(2-{[2-(trimethylsilyl)ethoxy]methyl}-2H-tetrazol-5-yl)phenyl]-1-(3-chlorophenyl)cyclopropanecarboxamide/N-[4-bromo-3-(1-{[2-(trimethyl-silyl)ethoxy]methyl}-1H-tetrazol-5-yl)phenyl]-1-(3-chlorophenyl)cyclopropanecarboxamide

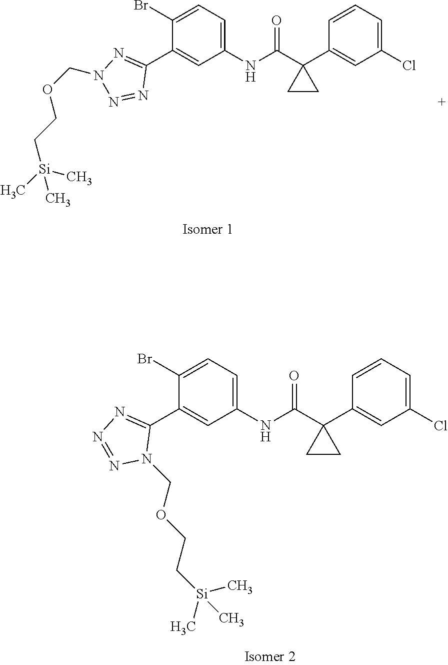 [SMILES: C[Si](C)(C)CCOCn1nnc(-c2cc(NC(=O)C3(c4cccc(Cl)c4)CC3)ccc2Br)n1 ; C[Si](C)(C)CCOCn1nnnc1-c1cc(NC(=O)C2(c3cccc(Cl)c3)CC2)ccc1Br]

To a room temperature solution of 4N-[4-bromo-3-(1H-tetrazol-5-yl)phenyl]-1-(3-chlorophenyl)cyclopropanecarboxamide (Intermediate 110A) (32.5 g, 77.6 mmol) in DMF (300 mL) was added dropwise 2-(trimethylsilyl)ethoxymethyl chloride (12.9 g, 77.6 mmol) and N,N-diisopropylethylamine (20.3 mL, 116 mmol) and stirred for 1h. The mixture was diluted with water and extracted with EE (×3). The combined organic layers were washed with brine, dried (Na₂SO₄), filtered and the solvents removed under reduced pressure. The crude product (1:1 mixture of regioisomers) was purified by flash chromatography (SiO₂, EE/hexane 0-15-305) to give N-[4-bromo-3-(2-{[2-(trimethylsilyl)ethoxy] methyl}-2H-tetrazol-5-yl)phenyl]-1-(3-chlorophenyl)cyclopropanecarboxamide (Isomer 1; 14.0 g, 33% yield) and N-[4-bromo-3-(1-{[2-(trimethyl silyl)ethoxy]methyl}-1H-tetrazol-5-yl)phenyl]-1-(3-chlorophenyl) cyclopropanecarboxamide (Isomer 2; 13.0 g, 31% yield).

### Isomer 1:

¹H NMR (400 MHz, DMSO-d6) δ[ppm]-0.05 (s, 9H), 0.89 (t, 2H), 1.16-1.20 (m, 2H), 1.48-1.51 (m, 2H), 3.74 (t, 2H), 6.07 (s, 2H), 7.36-7.38 (m, 3H), 7.44 (m, 1H), 7.74 (d, 2H), 8.14 (m, 1H), 9.56 (s, 1H).

LCMS (method 1): Rt=1.68 min, MS (ESipos) m/z=548 (M+H)⁺.

### Isomer 2:

¹H NMR (400 MHz, DMSO-d6) δ[ppm]-0.11 (s, 9H), 0.72-0.76 (m, 2H), 1.16-1.20 (m, 2H), 1.46-1.49 (m, 2H), 3.40-3.44 (m, 2H), 5.69 (s, 2H), 7.34-7.39 (m, 3H), 7.42 (m, 1H), 7.74 (dd, 2H), 7.79 (d, 1H), 7.88 (d, 1H), 9.50 (s, 1H).

LCMS (method 1): Rt=1.59 min, MS (ESipos) m/z=548 (M+H)⁺.

### Intermediate 112A: 2-(6-Ethoxypyridin-3-yl)-4-methyl-5-nitrobenzonitrile

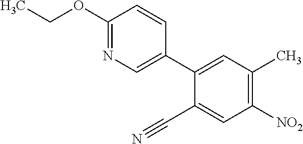 [SMILES: CCOc1ccc(-c2cc(C)c([N+](=O)[O-])cc2C#N)cn1]

A solution of 2-bromo-4-methyl-5-nitrobenzonitrile (2.00 g, 8.3 mmol), 2-ethoxy-5-(4,4,5,5-tetramethyl-1,3,2-dioxaborolan-2-yl)pyridine (2.07 g, 8.3 mmol) and potassium carbonate (3.78 g, 27 mmol) in dimethoxyethane (20 mL) and water (8 mL) was degassed with a stream of nitrogen for 10 mins. Dichlorobis(triphenylphosphine)palladium(II) (58 mg, 0.08 mmol) was added, and the reaction heated at 100° C. for 2 h under nitrogen. The reaction mixture was dissolved into ethyl acetate (250 mL) and washed with water (30 mL) then brine (30 mL), dried (Na₂SO₄), filtered and concentrated under reduced pressure. The residue was triturated with dichloromethane/heptane, and the precipitate collected by vacuum filtration. The mother liqueur was concentrated under reduced pressure then purified by flash chromatography (SiO₂, EE/heptane 0-100%). The batches were combined to give the title compound (1.71 g, 68% yield) as an off-white solid.

¹H NMR (500 MHz, DMSO-d6) δ[ppm] 1.36 (t, J=7.0 Hz, 3H), 2.64 (s, 3H), 4.40 (q, J=7.0 Hz, 2H), 7.00 (d, J=8.6 Hz, 1H), 7.86 (s, 1H), 8.03 (dd, J=8.6, 2.6 Hz, 1H), 8.46 (d, J=2.6 Hz, 1H), 8.64 (s, 1H).

LCMS (Analytical Method A): Rt=1.25 mins; MS (ESipos) m/z=284 (M+H)⁺.

In analogy to the procedure described for Intermediate 112A, the following intermediates were prepared using the corresponding boronic acid as starting material.

| Int. | Structure | Name | Analytical Data |
|---|---|---|---|
| 113A | 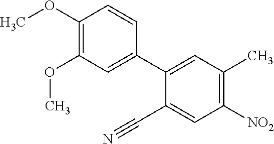 [SMILES: COc1ccc(-c2cc(C)c([N+](=O)[O-])cc2C#N)cc1OC] | 3′,4′-Dimethoxy- 5-methyl-4- nitrobiphenyl-2- carbonitrile | ¹H NMR (500 MHz, DMSO-d6) δ [ppm] 2.65 (s, 3H), 3.84 (s, 6H), 7.15 (d, J = 8.4 Hz, 1H), 7.19- 7.32 (m, 2H), 7.83 (s, 1H), 8.59 (s, 1H). LCMS (Analytical Method A): Rt = 1.19 mins, MS (ESIpos) low ionisation. |
| 114A | 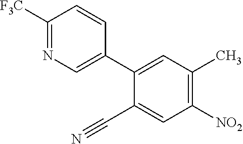 [SMILES: Cc1cc(-c2ccc(C(F)(F)F)nc2)c(C#N)cc1[N+](=O)[O-]] | 4-Methyl-5- nitro-2-[6- (trifluoromethyl) pyridin-3- yl]benzonitrile | ¹H NMR (500 MHz, Chloroform-d) δ [ppm] 8.90 (d, J = 2.0 Hz, 1H), 8.47 (s, 1H), 8.15 (dd, J = 8.1, 2.2 Hz, 1H), 7.88 (d, J = 8.1 Hz, 1H), 7.56 (s, 1H), 2.78 (s, 3H). LCMS (Analytical Method A): Rt = 1.20 mins; MS (ESIpos) m/z = 308 (M + H)⁺. |
| 115A | 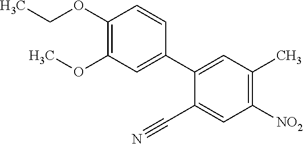 [SMILES: CCOc1ccc(-c2cc(C)c([N+](=O)[O-])cc2C#N)cc1OC] | 4′-Ethoxy-3′- methoxy-5- methyl-4- nitrobiphenyl-2- carbonitrile | ¹H NMR (500 MHz, DMSO-d6) δ [ppm] 1.37 (t, J = 7.0 Hz, 3H), 2.64 (s, 3H), 3.84 (s, 3H), 4.10 (q, J = 7.0 Hz, 2H), 7.13 (d, J = 8.4 Hz, 1H), 7.22 (dd, J = 8.3, 2.2 Hz, 1H), 7.27 (d, J = 2.2 Hz, 1H), 7.82 (s, 1H), 8.58 (s, 1H). LCMS (Analytical Method A): Rt = 1.26 mins, MS (ESIpos) low ionisation. |
| 116A | 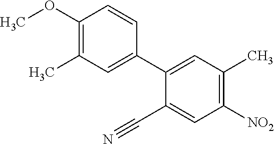 [SMILES: COc1ccc(-c2cc(C)c([N+](=O)[O-])cc2C#N)cc1C] | 4′-Methoxy-3′,5- dimethyl-4- nitrobiphenyl-2- carbonitrile | ¹H NMR (500 MHz, Chloroform-d) δ [ppm] 8.40 (s, 1H), 7.48 (s, 1H), 7.44 (dd, J = 8.4, 2.4 Hz, 1H), 7.35 (d, J = 2.3 Hz, 1H), 6.96 (d, J = 8.5 Hz, 1H), 3.91 (s, 3H), 2.73 (s, 3H), 2.30 (s, 3H). LCMS (Analytical Method A): Rt = 1.31 min, MS (ESIpos) low ionisation. |
| 117A | 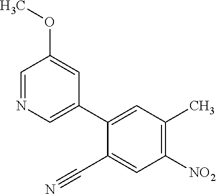 [SMILES: COc1cncc(-c2cc(C)c([N+](=O)[O-])cc2C#N)c1] | 2-(5- methoxypyridin- 3-yl)-4-methyl-5- nitrobenzonitrile | ¹H NMR (500 MHz, Chloroform-d) δ [ppm] 8.46 (d, J = 2.8 Hz, 1H), 8.44 (s, 1H), 8.37 (d, J = 1.8 Hz, 1H), 7.55 (s, 1H), 7.46-7.40 (m, 1H), 3.95 (s, 3H), 2.76 (s, 3H). LCMS (Analytical Method A): Rt = 1.08 min, MS (ESIpos) m/z = 270 [M + H]⁺ |

### Intermediate 118A: 2-Ethoxy-5-[5-methyl-4-nitro-2-(1H-tetrazol-5-yl)phenyl]pyridine

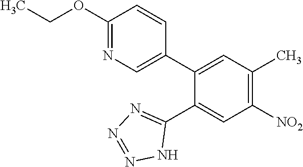 [SMILES: CCOc1ccc(-c2cc(C)c([N+](=O)[O-])cc2-c2nnn[nH]2)cn1]

2-(6-Ethoxypyridin-3-yl)-4-methyl-5-nitrobenzonitrile (1.71 g, 5.6 mmol) was split between two sealed tubes. To both tubes p-xylene (8 mL), di-n-butyltin oxide (700 mg, 2.8 mmol) and azidotrimethylsilane (1.12 mL, 8.4 mmol) were added. The resulting mixtures were heated in a sealed tube at 130° C. for 16 h. The reaction was cooled to RT, MeOH (10 mL) was added to both tubes and the mixture stirred at RT for 1 hour then reaction mixtures combined and concentrated at reduced pressure. The residual material was purified by flash chromatography (SiO₂, MeOH/DCM, 0-15%) to afford the title compound (1.41 g, 72% yield) as a yellow solid.

¹H NMR (500 MHz, DMSO-d6) δ[ppm] 1.32 (t, J=7.0 Hz, 3H), 2.66 (s, 3H), 4.32 (q, J=7.0 Hz, 2H), 6.77 (d, J=8.6 Hz, 1H), 7.44 (dd, J=8.6, 2.4 Hz, 1H), 7.75 (s, 1H), 8.03 (d, J=2.4 Hz, 1H), 8.41 (s, 1H).

LCMS (Analytical Method A): Rt=1.10 mins; MS (ESipos) m/z=327 (M+H)⁺.

In analogy to the procedure described for Intermediate 118A, the following intermediates were prepared using the corresponding nitrile as starting material.

| Int. | Structure | Name | Analytical Data |
|---|---|---|---|
| 119A | 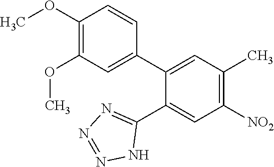 [SMILES: COc1ccc(-c2cc(C)c([N+](=O)[O-])cc2-c2nnn[nH]2)cc1OC] | 5-(3′,4′- Dimethoxy-5- methyl-4- nitrobiphenyl-2- yl)-1H-tetrazole | ¹H NMR (500 MHz, DMSO-d6) δ [ppm] 2.67 (s, 3H), 3.61 (s, 3H), 3.76 (s, 3H), 6.63-6.72 (m, 2H), 6.93 (d, J = 8.2 Hz, 1H), 7.74 (s, 1H), 8.30 (s, 1H). LCMS (Analytical Method A): Rt = 1.06 mins; MS (ESIpos) m/z = 342 (M + H)⁺. |
| 120A | 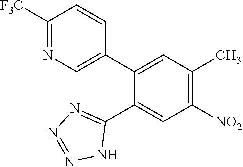 [SMILES: Cc1cc(-c2ccc(C(F)(F)F)nc2)c(-c2nnn[nH]2)cc1[N+](=O)[O-]] | 5-[5-Methyl-4- nitro-2-(1H- tetrazol-5- yl)phenyl]-2- (trifluoromethyl) pyridine | ¹H NMR (500 MHz, Methanol-d4) δ [ppm] 8.56 (d, J = 1.7 Hz, 1H), 8.52 (s, 1H), 7.90 (dd, J = 8.1, 1.7 Hz, 1H), 7.83 (d, J = 8.1 Hz, 1H), 7.71 (s, 1H), 2.74 (s, 3H). LCMS (Analytical Method A): Rt = 1.12 min; MS (ESIpos) m/z = 351 (M + H)⁺. |
| 121A | 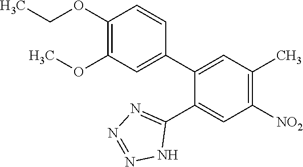 [SMILES: CCOc1ccc(-c2cc(C)c([N+](=O)[O-])cc2-c2nnn[nH]2)cc1OC] | 5-(4′-Ethoxy-3′- methoxy-5- methyl-4- nitrobiphenyl-2- yl)-1H-tetrazole | ¹H NMR (500 MHz, DMSO-d6) δ [ppm] 1.32 (t, J = 6.9 Hz, 3H), 2.67 (s, 3H), 3.61 (s, 3H), 4.01 (q, J = 6.9 Hz, 2H), 6.61-6.74 (m, 2H), 6.91 (d, J = 8.2 Hz, 1H), 7.74 (s, 1H), 8.29 (s, 1H). LCMS (Analytical Method A): Rt = 1.12 mins, MS (ESIpos) m/z = 356. |
| 122A | 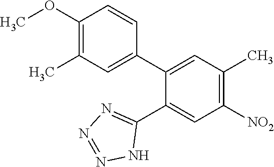 [SMILES: COc1ccc(-c2cc(C)c([N+](=O)[O-])cc2-c2nnn[nH]2)cc1C] | 5-(4′-Methoxy- 3′,5-dimethyl-4- nitrobiphenyl-2- yl)-1H-tetrazole | ¹H NMR (500 MHz, Chloroform-d) δ [ppm] 8.65 (s, 1H), 7.38 (s, 1H), 7.01-6.75 (m, 3H), 3.83 (s, 3H), 2.68 (s, 3H), 2.14 (s, 3H). LCMS (Analytical Method A): Rt = 1.16 min; MS (ESIpos) m/z = 326 (M + H)⁺. |
| 123A | 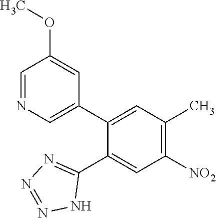 [SMILES: COc1cncc(-c2cc(C)c([N+](=O)[O-])cc2-c2nnn[nH]2)c1] | 3-methoxy-5-[5- methyl-4-nitro- 2-(1H-tetrazol-5- yl)phenyl]pyridine | ¹H NMR (500 MHz, Methanol-d4) δ 8.30 (s, 1H), 8.15 (d, J = 2.6 Hz, 1H), 7.94 (s, 1H), 7.65 (s, 1H), 7.17 (s, 1H), 3.77 (s, 3H), 2.70 (s, 3H). LCMS (Analytical Method A): Rt = 0.93 min, MS (ESIpos) m/z = 313 (M + H)⁺. |

### Intermediate 124A: 4-(6-ethoxypyridin-3-yl)-2-methyl-5-(1H-tetrazol-5-yl)aniline

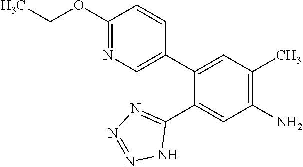 [SMILES: CCOc1ccc(-c2cc(C)c(N)cc2-c2nnn[nH]2)cn1]

To a solution of 2-ethoxy-5-[5-methyl-4-nitro-2-(1H-tetrazol-5-yl)phenyl]pyridine (1.41 g, 4.02 mmol) in ethanol (100 mL) was added 10% palladium on carbon (436 mg, 0.4 mmol) and the reaction stirred under an atmosphere of hydrogen for 4h. The reaction was filtered through Celite®, concentrated under reduced pressure then triturated with DCM/heptane to afford of the title compound (1.3 g, 91% yield).

¹H NMR (500 MHz, DMSO-d6) δ[ppm] 1.29 (t, J=7.0 Hz, 3H), 2.17 (s, 3H), 4.26 (q, J=7.0 Hz, 2H), 5.31 (s, 2H), 6.64 (d, J=8.5 Hz, 1H), 6.87 (s, 1H), 7.11 (s, 1H), 7.22 (dd, J=8.5, 2.5 Hz, 1H), 7.80 (d, J=2.2 Hz, 1H).

LCMS (Analytical Method A): Rt=0.95 mins; MS (ESIpos) m/z=297 (M+H)⁺.

In analogy to the procedure described for Intermediate 124A, the following intermediates were prepared using the corresponding nitro compound as starting material.

| Int. | Structure | Name | Analytical Data |
|---|---|---|---|
| 125A | 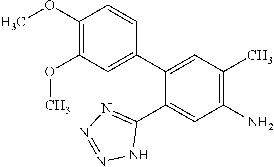 [SMILES: COc1ccc(-c2cc(C)c(N)cc2-c2nnn[nH]2)cc1OC] | 3′,4′-Dimethoxy- 5-methyl-2-(1H- tetrazol-5- yl)biphenyl-4- amine | ¹H NMR (250 MHz, DMSO-d6) δ [ppm] 2.17 (s, 3H), 3.56 (s, 3H), 3.71 (s, 3H), 5.23 (s, 2H), 6.41- 6.55 (m, 2H), 6.76-6.86 (m, 2H), 7.14 (s, 1H). LCMS (Analytical Method A): Rt = 0.91 mins; MS (ESIneg) m/z = 310 (M − H)⁻. |
| 126A | 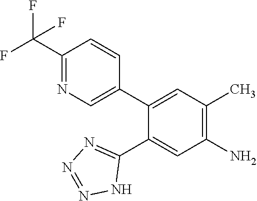 [SMILES: Cc1cc(-c2ccc(C(F)(F)F)nc2)c(-c2nnn[nH]2)cc1N] | 2-Methyl-5-(1H- tetrazol-5-yl)-4- [6-(trifluoromethyl) pyridin-3- yl]aniline | ¹H NMR (500 MHz, Methanol-d4) δ [ppm] 8.36 (s, 1H), 7.71-7.63 (m, 2H), 7.24 (s, 1H), 7.04 (s, 1H), 2.28 (s, 3H). LCMS (Analytical Method D): Rt = 3.54 min; MS (ESIpos) m/z = 321 (M + H)⁺. |
| 127A | 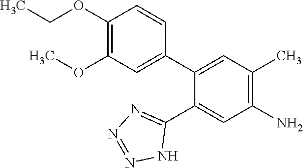 [SMILES: CCOc1ccc(-c2cc(C)c(N)cc2-c2nnn[nH]2)cc1OC] | 4′-Ethoxy-3′- methoxy-5- methyl-2-(1H- tetrazol-5- yl)biphenyl-4- amine | ¹H NMR (500 MHz, DMSO-d6) δ [ppm] 1.29 (t, J = 6.9 Hz, 3H), 3.55 (s, 3H), 3.95 (q, J = 6.8 Hz, 2H), 5.75 (s, 2H), 6.38-6.51 (m, 2H), 6.69-6.88 (m, 3H), 7.23 (d, J = 8.3 Hz, 1H). LCMS (Analytical Method A): Rt = 0.98 mins; MS (ESIpos) m/z = 326 (M + H)⁺. |
| 128A | 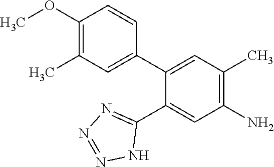 [SMILES: COc1ccc(-c2cc(C)c(N)cc2-c2nnn[nH]2)cc1C] | 4′-Methoxy-3′,5- dimethyl-2-(1H- tetrazol-5- yl)biphenyl-4- amine | ¹H NMR (500 MHz, Methanol-d4) δ [ppm] 7.17 (s, 1H), 6.95 (s, 1H), 6.84 (s, 1H), 6.82-6.74 (m, 2H), 3.81 (s, 3H), 2.28 (s, 3H), 2.12 (s, 3H). LCMS (Analytical Method A): Rt = 1.03 min; MS (ESIpos) m/z = 296 (M + H)⁺. |
| 129A | 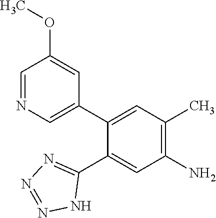 [SMILES: COc1cncc(-c2cc(C)c(N)cc2-c2nnn[nH]2)c1] | 4-(5- methoxypyridin- 3-yl)-2-methyl- 5-(1H-tetrazol- 5-yl)aniline | ¹H NMR (250 MHz, Methanol-d4) δ [ppm] 8.00 (d, J = 2.8 Hz, 1H), 7.82 (d, J = 1.7 Hz, 1H), 7.19 (s, 1H), 7.00 (dd, J = 2.7, 1.8 Hz, 1H), 6.97 (s, 1H), 3.72 (s, 3H), 2.27 (s, 3H). LCMS (Analytical Method A): Rt = 0.68 min; MS (ESIpos) m/z = 283 (M + H)⁺. |

### Intermediate 130A: 2-Bromo-3-methyl-5-nitrobenzonitrile

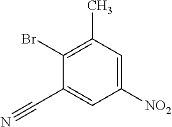

To a solution of 2-bromo-3-methylbenzonitrile (4.94 g, 25.2 mmol) in sulfuric acid (11 mL) was added nitric acid (1.68 mL, 37.8 mmol) dropwise, maintaining the temperature between 5-10° C. The reaction mixture turned into a brown slurry after complete addition. The reaction mixture was allowed to warm up to room temperature and was stirred overnight. The reaction mixture was then poured onto crushed ice and the resultant precipitate was collected by vacuum filtration, washed with water (100 mL) and dried in the vacuum oven at 40° C. for 6 hours to afford the title compound (4.95 g, 77% yield) as a brown solid.

¹H NMR (500 MHz, DMSO-d6) δ[ppm] 8.65 (d, J=2.5 Hz, 1H), 8.54 (d, J=2.5 Hz, 1H), 2.54 (s, 3H).

LCMS (Analytical Method A): Rt=1.15 min.

### Intermediate 131A: 3′,4′-Dimethoxy-6-methyl-4-nitrobiphenyl-2-carbonitrile

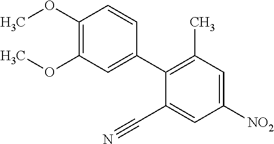 [SMILES: COc1ccc(-c2c(C)cc([N+](=O)[O-])cc2C#N)cc1OC]

A solution of 2-bromo-3-methyl-5-nitrobenzonitrile (3.0 g, 11.8 mmol), (3,4-dimethoxyphenyl)boronic acid (2.4 g, 13.0 mmol) and potassium carbonate (5.4 g, 39.0 mmol) in dimethoxyethane (20 mL) and water (15 mL) was heated at reflux under nitrogen atmosphere for 10 minutes. Dichlorobis(triphenylphosphine) palladium(II) (30 mg, 0.043 mmol) was added to the reaction and the mixture was heated at 100° C. for 2 hours. After cooling to RT, the reaction mixture was diluted with chloroform (100 mL) and water (30 mL). The organic layer was separated and the aqueous layer was extracted with chloroform (2×50 mL). The combined organic extracts were washed with brine (30 ml), dried (MgSO₄), filtered and concentrated under reduced pressure. The residue was suspended in MeOH (80 mL) and the mixture was briefly heated at reflux. After cooling to a RT, a brown precipitate was collected by filtration and dried in the vacuum oven at 40° C. for 4 hours to afford the title compound (4.16 g, 89% yield) as a brown solid.

¹H NMR (500 MHz, DMSO-d6) δ[ppm] 8.60 (d, J=2.3 Hz, 1H), 8.49 (d, J=1.9 Hz, 1H), 7.12 (d, J=8.3 Hz, 1H), 7.01 (d, J=2.0 Hz, 1H), 6.93 (dd, J=8.2, 2.0 Hz, 1H), 3.84 (s, 3H), 3.78 (s, 3H), 2.30 (s, 3H).

LCMS (Analytical Method A): Rt=1.18 mins; MS (ESipos) m/z=299 (M+H)⁺.

### Intermediate 132A: 5-(3′,4′-Dimethoxy-6-methyl-4-nitrobiphenyl-2-yl)-1H-tetrazole

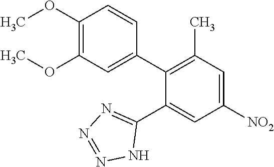 [SMILES: COc1ccc(-c2c(C)cc([N+](=O)[O-])cc2-c2nnn[nH]2)cc1OC]

Two ACE pressure tubes were loaded each with 3′,4′-dimethoxy-6-methyl-4-nitrobiphenyl-2-carbonitrile (1.45 g, 4.62 mmol), p-xylene (8 ml), di-n-butyltin oxide (1.15 g, 4.62 mmol) and azidotrimethylsilane (705 μl, 5.31 mmol). The pressure tubes were sealed and heated at 130° C. for 2 hours. The reaction was cooled to RT, MeOH (10 mL) was added and the mixtures stirred at RT for 1 hour, then combined and concentrated at reduced pressure. The crude material was purified by flash chromatography (SiO₂, MeOH/DCM, 0-20%) to afford the title compound (2.61 g, 79% yield) as a brown solid.

¹H NMR (500 MHz, DMSO-d6) δ[ppm] 8.41 (d, J=2.2 Hz, 1H), 8.36 (d, J=2.4 Hz, 1H), 6.90 (d, J=8.3 Hz, 1H), 6.67 (d, J=2.0 Hz, 1H), 6.56 (dd, J=8.2, 2.0 Hz, 1H), 3.75 (s, 3H), 3.65 (s, 3H), 2.30 (s, 3H).

LCMS (Analytical Method A): Rt=1.05 mins; MS (ESipos) m/z=342 (M+H)⁺.

### Intermediate 133A: 3′,4′-Dimethoxy-2-methyl-6-(1H-tetrazol-5-yl)biphenyl-4-amine

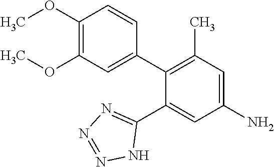 [SMILES: COc1ccc(-c2c(C)cc(N)cc2-c2nnn[nH]2)cc1OC]

To a de-gassed suspension of 5-(3′,4′-dimethoxy-6-methyl-4-nitrobiphenyl-2-yl)-1H-tetrazole (2.60 g, 2.14 mmol) in EtOH (60 mL) was added Pd/C (5% w/w; 250 mg, 0.12 mmol). The mixture was stirred at room temperature under an atmosphere of hydrogen for a total of 16 hours. The catalyst was removed by filtration through Celite®, washing with EtOH (50 mL). The filtrate was concentrated in vacuo, the residue dissolved in DCM (20 ml) and TBME (100 ml) was added slowly with stirring. A yellow precipitate formed and was collected by filtration and dried in the vacuum oven at 40° C. for 6 hours to afford the title compound (2.25 g, 99% yield) as a yellow powder.

¹H NMR (500 MHz, DMSO-d6) δ[ppm] 6.78 (d, J=8.3 Hz, 1H), 6.68 (d, J=2.2 Hz, 1H), 6.57 (d, J=2.3 Hz, 1H), 6.52 (d, J=2.0 Hz, 1H), 6.43 (dd, J=8.2, 2.0 Hz, 1H), 5.35 (s, 2H), 3.70 (s, 3H), 3.61 (s, 3H), 2.03 (s, 3H).

LCMS (Analytical Method F): Rt=1.81 mins; MS (ESIpos) m/z=312 (M+H)⁺.

Intermediate 134A: 5-fluoro-3′,4′-dimethoxy-4-nitrobiphenyl-2-carbonitrile

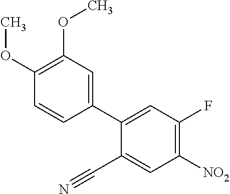 [SMILES: COc1ccc(-c2cc(F)c([N+](=O)[O-])cc2C#N)cc1OC]

Water (37 mL) and 1,2-dimethoxyethane (74 mL) were degassed with a stream of argon for 15 mins. Then 2-bromo-4-fluoro-5-nitrobenzonitrile (5.00 g, 20.4 mmol), 3,4-dimethylbenzeneboronic acid (3.71 g, 20.4 mmol), potassium carbonate (9.31 g, 67.3 mmol) and dichlorobis(triphenylphosphine) palladium(II) (172 mg, 0.245 mmol) were added, and the reaction heated at 90° C. for 5 h. The mixture was cooled and poured into water. Ethyl acetate was added and the layers separated. The aqueous layer was extracted with ethyl acetate (×3). The combined organic layers were washed with brine, dried (Na₂SO₄) and concentrated under reduced pressure. The residue was purified by flash chromatography (SiO₂, EE/hexane 0-30-50%) to yield of the desired product (2.96 g, 38% yield).

¹H NMR (400 MHz, DMSO-d) 6 [ppm] 3.34 (s, 3H), 3.86 (s, 3H), 7.18 (d, 1H), 7.30-7.33 (m, 2H), 8.00 (d, 1H), 8.84 (d, 1H); LCMS (method 1): Rt=1.09 min, MS (ESipos) m/z=303 (M+H)⁺.

In analogy to the procedure described for Intermediate 134A, the following intermediates were prepared using the corresponding boronic acid as starting material.

| Int. | Structure | Name | Analytical Data |
|---|---|---|---|
| 135A | 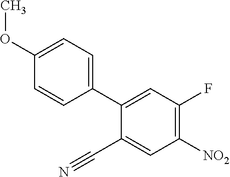 [SMILES: COc1ccc(-c2cc(F)c([N+](=O)[O-])cc2C#N)cc1] | 5-fluoro-4′- methoxy-4- nitrobiphenyl-2- carbonitrile | ¹H NMR (400 MHz, DMSO-d) δ [ppm] 3.86 (s, 3H), 7.16 (d, 2H), 7.69 (d, 2H), 7.94 (d, 1H), 8.84 (d, 1H). LCMS (method 1): Rt = 1.15 min; MS (ESIpos) m/z = 273 (M + H)⁺ |
| 136A | 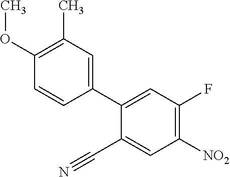 [SMILES: COc1ccc(-c2cc(F)c([N+](=O)[O-])cc2C#N)cc1C] | 5-fluoro-4′- methoxy-3′- methyl-4- nitrobiphenyl-2- carbonitrile | ¹H NMR (400 MHz, DMSO-d) δ [ppm] 2.24 (s, 3H), 3.89 (s, 3H), 7.16 (d, 1H), 7.53 (m, 1H), 7.59 (dd, 1H), 7.92 (d, 1H), 8.83 (d, 1H). LCMS (method 1): Rt = 1.27 min; MS (ESIpos) m/z = 287 (M + H)⁺ |

### Intermediate 137A: 5-(5-fluoro-3′,4′-dimethoxy-4-nitrobiphenyl-2-yl)-1H-tetrazole

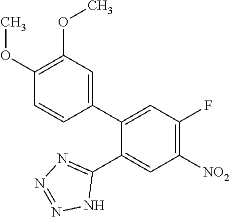 [SMILES: COc1ccc(-c2cc(F)c([N+](=O)[O-])cc2-c2nnn[nH]2)cc1OC]

5-Fluoro-3′,4′-dimethoxy-4-nitrobiphenyl-2-carbonitrile (Intermediate 134A; 710 mg, 2.35 mmol) were dissolved in toluene (25 mL) and azidotrimethylsilane (1.25 mL, 9.37 mmol) and di-n-butyl tin oxide (877 mg, 3.52 mmol) were added at room temperature. The mixture was heated to 130° C. (bath temperature) for 4 h. The mixture was cooled and diluted with methanol. Silica gel was added and the solvents evaporated under reduced pressure. The residue was directly purified by chromatography (SiO₂, MeOH/DCM 0-10-30%) to yield the title compound (720 mg, 89% yield) as a yellow solid.

¹H NMR (400 MHz, DMSO-d6) δ[ppm] 3.63 (s, 3H), 3.77 (s, 3H), 6.69 (dd, 1H), 6.75 (d, 1H), 6.94 (d, 1H), 7.90 (d, 1H), 8.47 (d, 1H).

LCMS (method 2): Rt=0.90 min; MS (ESipos) m/z=346 (M+H)⁺.

In analogy to the procedure described for Intermediate 137A, the following intermediates were prepared using the corresponding nitrile as starting material.

| Int. | Structure | Name | Analytical Data |
|---|---|---|---|
| 138A | 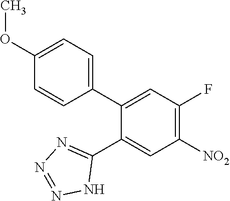 [SMILES: COc1ccc(-c2cc(F)c([N+](=O)[O-])cc2-c2nnn[nH]2)cc1] | 5-(5-fluoro-4′- methoxy-4- nitrobiphenyl-2- yl)-1H-tetrazole | ¹H NMR (400 MHz, DMSO-d6) δ [ppm] 3.77 (s, 3H), 6.90 (d, 2H), 7.12 (d, 2H), 7.72 (d, 1H), 8.39 (d, 1H). LCMS (method 1): Rt = 0.95 min; MS (ESIpos) m/z = 316 (M + H)⁺ |
| 139A | 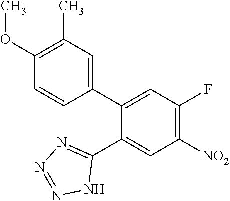 [SMILES: COc1ccc(-c2cc(F)c([N+](=O)[O-])cc2-c2nnn[nH]2)cc1C] | 5-(5-fluoro-4′- methoxy-3′- methyl-4- nitrobiphenyl-2- yl)-1H-tetrazole | ¹H NMR (400 MHz, DMSO-d) δ [ppm] 2.10 (s, 3H), 3.80 (s, 3H), 6.89-6.90 (m, 2H), 7.05 (m, 1H), 7.82 (d, 1H), 8.47 (d, 1H). LCMS (method 1): Rt = 1.06 min; MS (ESIpos) m/z = 330 (M + H)⁺ |

### Intermediate 140A: 5-fluoro-3′,4′-dimethoxy-2-(1H-tetrazol-5-yl)biphenyl-4-amine

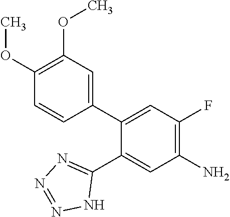 [SMILES: COc1ccc(-c2cc(F)c(N)cc2-c2nnn[nH]2)cc1OC]

5-(5-Fluoro-3′,4′-dimethoxy-4-nitrobiphenyl-2-yl)-1H-tetrazole (Intermediate 137A; 720 mg, 2.09 mmol) was dissolved in ethanol (12 mL) and palladium on carbon (10% w/w, 220 mg) added. The mixture was shaken under hydrogen atmosphere for 3 h. Then the catalyst was removed by filtration through Celite® and washed with EtOH and DCM. The filtrate was evaporated to dryness under reduced pressure. The title compound (640 mg, 90% yield) was afforded as an off-white foam and used without further purification.

¹H NMR (400 MHz, DMSO-d6) δ[ppm] 3.58 (s, 3H), 3.72 (s, 3H), 6.50 (dd, 1H), 6.53 (d, 1H), 6.83 (d, 1H), 6.98 (d, 1H), 7.25 (d, 1H).

LCMS (method 1): Rt=0.74 min; MS (ESipos) m/z=316 (M+H)⁺.

In analogy to the procedure described for Intermediate 140A, the following intermediates were prepared using the corresponding nitro compound as starting material.

| Int. | Structure | Name | Analytical Data |
|---|---|---|---|
| 141A | 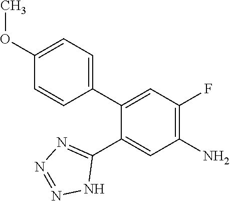 [SMILES: COc1ccc(-c2cc(F)c(N)cc2-c2nnn[nH]2)cc1] | 5-fluoro-4′- methoxy-2-(1H- tetrazol-5- yl)biphenyl-4- amine | ¹H NMR (400 MHz, DMSO-d6) δ [ppm] 3.72 (s, 3H), 6.81 (d, 2H), 6.91 (d, 2H), 6.97 (d, 1H), 7.17 (d, 1H). LCMS (method 1): Rt = 0.79 min; MS (ESIpos) m/z = 286 (M + H)⁺. |
| 142A | 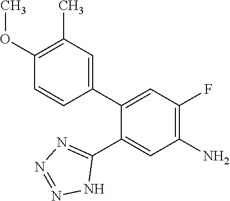 [SMILES: COc1ccc(-c2cc(F)c(N)cc2-c2nnn[nH]2)cc1C] | 5-fluoro-4′- methoxy-3′- methyl-2-(1H- tetrazol-5- yl)biphenyl-4- amine | ¹H NMR (400 MHz, DMSO-d) δ [ppm] 2.06 (s, 3H), 3.75 (s, 3H), 6.69 (dd, 1H), 6.78 (d, 1H), 6.85 (d, 1H), 6.97 (d, 1H), 7.16 (d, 1H). LCMS (method 1): Rt = 0.91 min; MS (ESIpos) m/z = 300 (M + H)⁺. |

### Intermediate 143A: 5-(2-bromo-4-fluoro-5-nitrophenyl)-1H-tetrazole

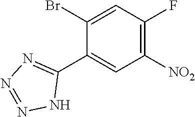 [SMILES: O=[N+]([O-])c1cc(-c2nnn[nH]2)c(Br)cc1F]

5-(2-bromo-4-fluoro-5-nitrophenyl)-1H-tetrazole was prepared in analogy to Intermediates 130A (nitration)/137A (formation of tetrazole).

¹H NMR (400 MHz, DMSO-d6) δ[ppm] 8.19 (d, 1H), 8.48 (d, 1H).

LCMS (method 1): Rt=0.68 min; MS (ESipos) m/z=288 (M+H)⁺.

### Intermediate 144A: 4-bromo-2-fluoro-5-(1H-tetrazol-5-yl)aniline

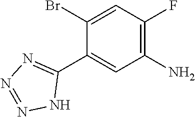 [SMILES: Nc1cc(-c2nnn[nH]2)c(Br)cc1F]

5-(2-Bromo-4-fluoro-5-nitrophenyl)-1H-tetrazole (Intermediate 143A; 18.0 g, 62.5 mmol) were dissolved in ethanol (350 mL), palladium on carbon (10% w/w; 6.65 g) were added and the mixture stirred at room temperature under a hydrogen atmosphere (1 atmosphere) until complete conversion. The catalyst was removed by filtration through Celite®, rinsed with ethanol and DCM and the solvents removed under reduced pressure. The title compound was used without further purification.

¹H NMR (400 MHz, DMSO-d6) δ[ppm] 7.08 (d, 1H), 7.43 (d, 1H).

LCMS (method 1): Rt=0.57 min; MS (ESipos) m/z=258 (M+H)⁺.

### Intermediate 145A: N-[4-bromo-2-fluoro-5-(1H-tetrazol-5-yl)phenyl]-1-(3-chlorophenyl) cyclopropanecarboxamide

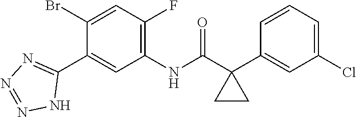 [SMILES: O=C(Nc1cc(-c2nnn[nH]2)c(Br)cc1F)C1(c2cccc(Cl)c2)CC1]

The compound was prepared in analogy to Intermediate 110A.

¹H NMR (400 MHz, DMSO-d6) δ[ppm] 1.18-1.22 (m, 2H), 1.48-1.50 (m, 2H), 7.40-7.52 (m, 4H), 7.78 (d, 1H), 7.86 (d, 1H), 8.90 (s, 1H).

LCMS (method 2): Rt=1.15 min; MS (ESipos) m/z=435/437/439 (M+H, ClBr isotope pattern)⁺.

### Intermediate 146A: N-[4-bromo-2-fluoro-5-(2-{[2-(trimethylsilyl)ethoxy]methyl}-2H-tetrazol-5-yl)phenyl]-1-(3-chlorophenyl)cyclopropanecarboxamide/N-[4-bromo-2-fluoro-5-(1-{[2-(trimethylsilyl)ethoxy]methyl}-1H-tetrazol-5-yl)phenyl]-1-(3-chlorophenyl)cyclopropanecarboxamide

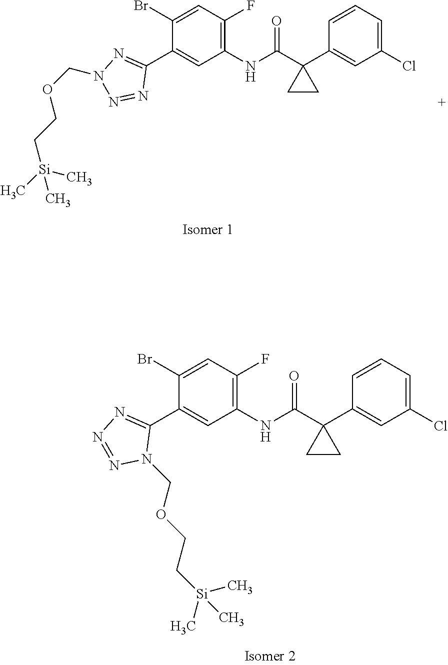 [SMILES: C[Si](C)(C)CCOCn1nnc(-c2cc(NC(=O)C3(c4cccc(Cl)c4)CC3)c(F)cc2Br)n1 ; C[Si](C)(C)CCOCn1nnnc1-c1cc(NC(=O)C2(c3cccc(Cl)c3)CC2)c(F)cc1Br]

The compound was prepared in analogy to Intermediate 111A.

Isomer 1: (N-[4-bromo-2-fluoro-5-(2-{[2-(trimethylsilyl)ethoxy]methyl}-2H-tetrazol-5-yl)phenyl]-1-(3-chlorophenyl)cyclopropanecarboxamide):

¹H NMR (400 MHz, DMSO-d6) δ[ppm]-0.05 (s, 9H), 0.89 (t, 2H), 1.20-1.22 (m, 2H), 1.49-1.51 (m, 2H), 3.74 (t, 2H), 6.08 (s, 2H), 7.40-7.44 (m, 3H), 7.50 (m, 1H), 7.87 (d, 1H), 8.07 (m, 1H), 8.88 (s, 1H).

Isomer 2: N-[4-bromo-2-fluoro-5-(1-{[2-(trimethylsilyl)ethoxy]methyl}-1H-tetrazol-5-yl)phenyl]-1-(3-chlorophenyl)cyclopropanecarboxamide):

¹H NMR (400 MHz, DMSO-d6) δ[ppm]-0.09 (s, 9H), 0.76 (t, 2H), 1.18-1.22 (m, 2H), 1.46-1.49 (m, 2H), 3.44 (t, 2H), 5.67 (s, 2H), 7.40-7.43 (m, 3H), 7.47 (m, 1H), 7.87 (d, 1H), 7.94 (d, 1H), 8.93 (s, 1H).

### Intermediate 147A: 4-bromo-3-(2-{[2-(trimethylsilyl)ethoxy]methyl}-2H-tetrazol-5-yl)aniline/4-bromo-3-(1-{[2-(trimethylsilyl)ethoxy]methyl}-1H-tetrazol-5-yl)aniline

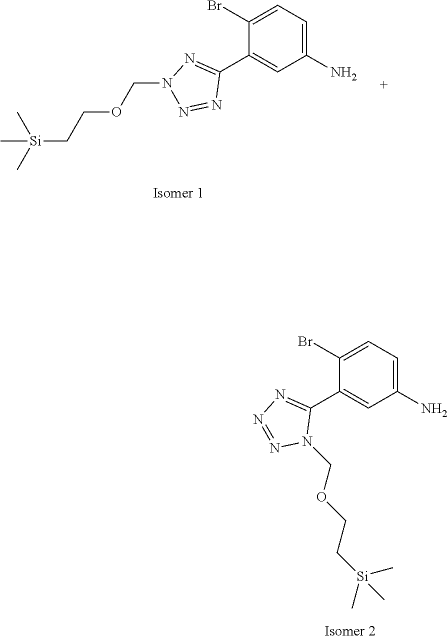 [SMILES: C[Si](C)(C)CCOCn1nnc(-c2cc(N)ccc2Br)n1 ; C[Si](C)(C)CCOCn1nnnc1-c1cc(N)ccc1Br]

The compound was prepared from Intermediate 109A in analogy to Intermediate 111A and used as a mixture of isomers.

Isomer 1:

¹H NMR (400 MHz, DMSO-d6) δ[ppm]-0.05 (s, 9H), 0.89 (t, 2H), 3.74 (t, 2H), 5.60 (s, br, 2H), 6.05 (s, 2H), 6.67 (dd, 1H), 7.02 (d, 1H), 7.39 (d, 1H).

LCMS (method 1): Rt=1.35 min, MS (ESipos) m/z=370 (M+H, Br isotope pattern)⁺.

Isomer 2:

¹H NMR (400 MHz, DMSO-d6) δ[ppm]-0.11 (s, 9H), 0.72-0.80 (m, 2H), 3.43-3.49 (m, 2H), 5.66 (s, 2H), 5.67 (s, br, 2H), 6.70 (d, 1H), 6.75 (dd, 1H), 7.43 (d, 1H).

LCMS (method 1): Rt=1.26 min, MS (ESIpos) m/z=370 (M+H, Br isotope pattern)⁺.

### Intermediate 148A: 4′-amino-3-fluoro-2′-(2H-tetrazol-5-yl)biphenyl-4-carbonitrile

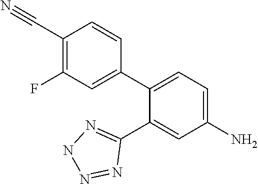 [SMILES: N#Cc1ccc(-c2ccc(N)cc2-c2nn[nH]n2)cc1F]

A solution of 4-bromo-3-(2-{[2-(trimethylsilyl)ethoxy]methyl}-2H-tetrazol-5-yl)aniline (and/or the respective 1H-isomer) (1.0 g, 2.70 mmol), 4-cyano-3-fluorobenzene boronic acid (445 mg, 2.70 mmol) and potassium carbonate (1.23 g, 8.91 mmol) in 1,2-dimethoxyethane (10 mL) and water (5 mL) was degassed with a stream of argon for 5 mins.

Dichlorobis(triphenylphosphine)palladium(II) (23 mg, 0.032 mmol) was added and the reaction heated at 90° C. for 2 h. The reaction was then cooled to RT and diluted with EE (50 mL) and the aqueous layer was removed. The organics were washed with brine (2×40 mL), dried (Na₂SO₄), filtered and concentrated to give the SEM-protected intermediate, which was used without further purification.

LCMS (method 3) Rt=1.32 min and 1.39 min (mixture of isomers), MS (ESipos) m/z=281 (M+H)⁺.

The crude intermediate was re-dissolved in methanol (3 mL) and hydrochloric acid (2 mL, 3M) and stirred at 70° C. for 4 h. After cooling to RT, the mixture was concentrated to dryness and purified by chromatography (SiO₂, DCM/MeOH 0-10-20%) to give 520 mg (66% yield) of the title compound as a yellow foam.

¹H NMR (400 MHz, DMSO-d6) δ[ppm] 6.86-6.90 (m, 3H), 7.21 (dd, 1H), 7.34 (d, 1H) 7.76 (dd, 1H).

LCMS (method 1): Rt=0.73 min, MS (ESipos) m/z=281 (M+H, Br isotope pattern)⁺.

### Intermediate 149A: 2-Bromo-3-fluoro-5-nitrobenzonitrile

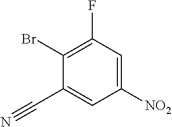 [SMILES: N#Cc1cc([N+](=O)[O-])cc(F)c1Br]

To a solution of 2-bromo-3-fluorobenzonitrile (10.0 g, 50 mmol) in sulfuric acid (22 mL) was added nitric acid (69%, 4.82 mL, 75 mmol) dropwise, maintaining the temperature between 5-10° C. The reaction mixture turned red-orange at the end of the addition. The reaction mixture was allowed to warm up to RT and was stirred for 1 hour. The reaction mixture was then poured onto crushed ice. The products were extracted with EtOAc (2×100 mL). The combined organic layers were washed with brine (50 mL), dried (MgSO₄), filtered and concentrated under reduced pressure. The residue was suspended in MeOH (50 mL) and the mixture was briefly heated at reflux. After cooling to RT, a pale yellow precipitate was collected by vacuum filtration, washed with a small amount of MeOH and dried in the vacuum oven. The material was recrystallized from MeOH to afford 1.77 g (14% yield) of the title compound as a yellow powder.

¹H NMR (500 MHz, DMSO-d6) δ[ppm] 8.77 (dd, J=2.5, 1.4 Hz, 1H), 8.65 (dd, J=8.3, 2.5 Hz, 1H).

LCMS (Analytical Method A): Rt=1.11 min, mass ion not observed.

### Intermediate 150A: 6-Fluoro-3′,4′-dimethoxy-4-nitrobiphenyl-2-carbonitrile

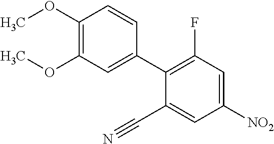 [SMILES: COc1ccc(-c2c(F)cc([N+](=O)[O-])cc2C#N)cc1OC]

A mixture of 2-bromo-3-fluoro-5-nitrobenzonitrile (1.6 g, 6.2 mmol), (3,4-dimethoxyphenyl)boronic acid (1.2 g, 6.8 mmol) and potassium carbonate (2.8 g, 20.5 mmol) in dimethoxyethane (11 mL) and water (8 mL) was heated at reflux under nitrogen atmosphere for 10 minutes. Dichlorobis(triphenyl phosphine) palladium(II) (30 mg, 0.043 mmol) was added to the reaction and the mixture was heated at 100° C. for 2 hours. After cooling to RT, the reaction mixture was diluted with chloroform (100 mL) and water (30 mL).

The organic layer was separated and the aqueous layer extracted with chloroform (2×50 mL). The combined organic extracts were washed with brine (30 mL), dried (MgSO₄), filtered and concentrated under reduced pressure. The crude material was purified by flash chromatography (SiO₂, DCM/EtOAc, 0-100%) to afford 1.71 g (90% yield) of the title compound as an orange solid.

¹H NMR (500 MHz, DMSO-d6) δ[ppm] 8.75-8.69 (m, 1H), 8.58 (dd, J=9.3, 2.2 Hz, 1H), 7.23 (s, 1H), 7.19-7.14 (m, 2H), 3.85 (s, 3H), 3.80 (s, 3H).

LCMS (Analytical Method A): Rt=4.18 mins.

In analogy to the procedure described for Intermediate 150A, the following intermediates were prepared using the corresponding boronic acid as starting material.

| Int. | Structure | Name | Analytical Data |
|---|---|---|---|
| 151A | 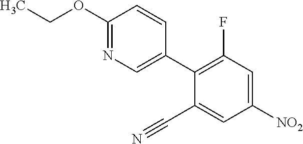 [SMILES: CCOc1ccc(-c2c(F)cc([N+](=O)[O-])cc2C#N)cn1] | 2-(6- ethoxypyridin-3- yl)-3-fluoro-5- nitrobenzonitrile | ¹H NMR (500 MHz, DMSO-d6) δ [ppm] 8.84-8.74 (m, 1H), 8.64 (dd, J = 9.3, 2.2 Hz, 1H), 8.46- 8.38 (m, 1H), 7.98 (ddd, J = 8.6, 2.4, 1.2 Hz, 1H), 7.03 (d, J = 8.6 Hz, 1H), 4.40 (q, J = 7.0 Hz, 2H), 1.36 (t, J = 7.0 Hz, 3H). LCMS (Analytical Method A): Rt = 1.24 mins; MS (ESIpos) m/z = 287.9 (M + H)⁺. |
| 152A | 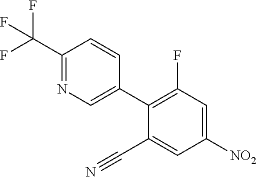 [SMILES: N#Cc1cc([N+](=O)[O-])cc(F)c1-c1ccc(C(F)(F)F)nc1] | 3-fluoro-5-nitro- 2-[6- (trifluoromethyl) pyridin-3- yl]benzonitrile | ¹H NMR (500 MHz, DMSO-d6) δ 9.06 (s, 1H), 8.93-8.85 (m, 1H), 8.75 (dd, J = 9.2, 2.2 Hz, 1H), 8.51-8.40 (m, 1H), 8.22 (d, J = 8.2 Hz, 1H). LCMS (Analytical Method A): Rt = 1.21 mins; MS (ESIpos) m/z = 312 (M + H)⁺. |
| 153A | 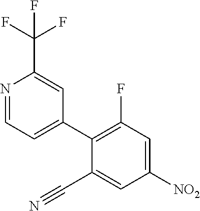 [SMILES: N#Cc1cc([N+](=O)[O-])cc(F)c1-c1ccnc(C(F)(F)F)c1] | 3-fluoro-5-nitro- 2-[2- (trifluoromethyl) pyridin-4- yl]benzonitrile | ¹H NMR (500 MHz, DMSO-d6) δ 9.05 (d, J = 4.9 Hz, 1H), 8.88 (s, 1H), 8.74 (dd, J = 9.3, 2.1 Hz, 1H), 8.30 (s, 1H), 8.28 (d, J = 5.1 Hz, 0H), 8.05 (d, J = 4.9 Hz, 1H). LCMS (Analytical Method A): Rt = 1.21 min; MS (ESIpos) m/z = 312 (M + H)⁺. |
| 154A | 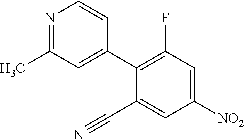 [SMILES: Cc1cc(-c2c(F)cc([N+](=O)[O-])cc2C#N)ccn1] | 3-fluoro-2-(2- methylpyridin- 4-yl)-5- nitrobenzonitrile | ¹H NMR (500 MHz, DMSO-d6) δ [ppm] 8.84-8.80 (m, 1H), 8.71- 8.66 (m, 2H), 7.50 (s, 1H), 7.43 (d, J = 5.0 Hz, 1H), 2.58 (s, 3H). LCMS (Analytical Method A): Rt = 0.91 mins; MS (ESIpos) m/z = 258 (M + H)⁺. |
| 155A | 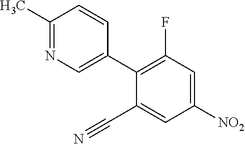 [SMILES: Cc1ccc(-c2c(F)cc([N+](=O)[O-])cc2C#N)cn1] | 3-fluoro-2-(6- methylpyridin- 3-yl)-5- nitrobenzonitrile | ¹H NMR (500 MHz, DMSO-d6) δ [ppm] 8.82-8.79 (m, 1H), 8.68- 8.64 (m, 2H), 7.99-7.95 (m, 1H), 7.51 (d, J = 8.1 Hz, 1H), 2.59 (s, 3H). LCMS (Analytical Method A): Rt = 1.03 mins; MS (ESIpos) m/z = 258 (M + H)⁺. |

### Intermediate 156A: 5-(6-Fluoro-3′,4′-dimethoxy-4-nitrobiphenyl-2-yl)-1H-tetrazole

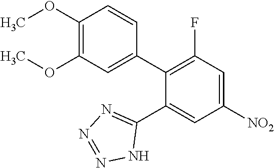 [SMILES: COc1ccc(-c2c(F)cc([N+](=O)[O-])cc2-c2nnn[nH]2)cc1OC]

An ACE pressure tube was loaded with 6-fluoro-3′,4′-dimethoxy-4-nitrobiphenyl-2-carbonitrile (1.7 g, 5.6 mmol), p-xylene (10 mL), di-n-butyltin oxide (1.4 g, 5.5 mmol) and azidotrimethylsilane (850 μL, 6.4 mmol). The pressure tube was sealed and heated at 130° C. for 2 hours. The reaction was cooled to RT, MeOH (10 mL) was added and the mixture stirred at RT for 1 hour, then concentrated at reduced pressure. The crude material was purified by flash chromatography (SiO₂, MeOH/DCM, 0-20%) to afford 1.33 g (68% yield) of the title compound as a yellow solid.

¹H NMR (500 MHz, DMSO-d6) δ[ppm] 8.47-8.43 (m, 2H), 6.96 (d, J=8.3 Hz, 1H), 6.76-6.69 (m, 2H), 3.77 (s, 3H), 3.61 (s, 3H).

LCMS (Analytical Method A): Rt=1.03 mins; MS (ESipos) m/z=346.0 (M+H)⁺.

In analogy to the procedure described for Intermediate 156A, the following intermediates were prepared using the corresponding nitrile as starting material.

| Int. | Structure | Name | Analytical Data |
|---|---|---|---|
| 157A | 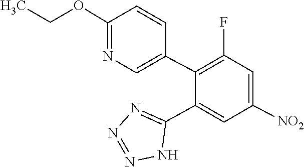 [SMILES: CCOc1ccc(-c2c(F)cc([N+](=O)[O-])cc2-c2nnn[nH]2)cn1] | 2-ethoxy-5-[2- fluoro-4-nitro-6- (1H-tetrazol-5- yl)phenyl] pyridine | ¹H NMR (500 MHz, DMSO-d6) δ [ppm] 8.60-8.49 (m, 1H), 8.39 (dd, J = 9.1, 2.3 Hz, 1H), 8.00 (d, J = 2.3 Hz, 1H), 7.54 (dd, J = 8.6, 2.4 Hz, 1H), 6.82 (d, J = 8.6 Hz, 1H), 4.32 (q, J = 7.0 Hz, 2H), 1.33 (t, J = 7.0 Hz, 3H). LCMS (Analytical Method A): Rt = 1.11 mins; MS (ESIpos) m/z = 331 (M + H)⁺. |
| 158A | 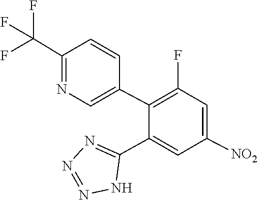 [SMILES: O=[N+]([O-])c1cc(F)c(-c2ccc(C(F)(F)F)nc2)c(-c2nnn[nH]2)c1] | 5-[2-fluoro-4- nitro-6-(1H- tetrazol-5- yl)phenyl]-2- (trifluoromethyl) pyridine | ¹H NMR (500 MHz, DMSO-d6) δ [ppm] 8.69 (s, 2H), 8.49 (d, J = 8.1 Hz, 1H), 8.08 (dd, J = 8.1, 1.7 Hz, 1H), 8.01 (d, J = 8.1 Hz, 1H). LCMS (Analytical Method A): Rt = 1.11 mins; MS (ESIpos) m/z = 355 (M + H)⁺. |
| 159A | 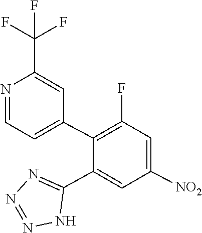 [SMILES: O=[N+]([O-])c1cc(F)c(-c2ccnc(C(F)(F)F)c2)c(-c2nnn[nH]2)c1] | 4-[2-fluoro-4- nitro-6-(1H- tetrazol-5- yl)phenyl]-2- (trifluoromethyl) pyridine | ¹H NMR (500 MHz, DMSO-d6) δ 8.83 (d, J = 4.9 Hz, 1H), 8.73- 8.65 (m, 1H), 8.53-8.42 (m, 1H), 7.92 (s, 1H), 7.67 (d, J = 4.9 Hz, 1H). LCMS (Analytical Method A): Rt = 1.12 min; MS (ESIpos) m/z = 355 (M + H)⁺ |
| 160A | 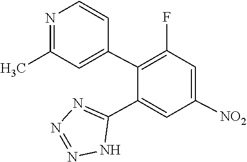 [SMILES: Cc1cc(-c2c(F)cc([N+](=O)[O-])cc2-c2nnn[nH]2)ccn1] | 4-[2-fluoro-4- nitro-6-(1H- tetrazol-5- yl)phenyl]-2- methylpyridine | ¹H NMR (500 MHz, DMSO-d6) δ [ppm] 8.65-8.58 (m, 1H), 8.51 (d, J = 5.2 Hz, 1H), 8.49 (dd, J = 9.1, 2.2 Hz, 1H), 7.26 (s, 1H), 7.11 (d, J = 5.0 Hz, 1H), 2.50 (s, 3H). LCMS (Analytical Method A): Rt = 0.78 mins; MS (ESIpos) m/z = 301.1 (M + H)⁺. |
| 161A | 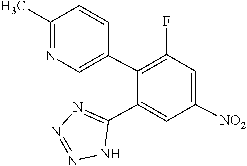 [SMILES: Cc1ccc(-c2c(F)cc([N+](=O)[O-])cc2-c2nnn[nH]2)cn1] | 5-[2-fluoro-4- nitro-6-(1H- tetrazol-5- yl)phenyl]-2- methylpyridine | ¹H NMR (500 MHz, DMSO-d6) δ [ppm] 8.62-8.54 (m, 1H), 8.47 (dd, J = 9.1, 2.1 Hz, 1H), 8.30 (d, J = 1.6 Hz, 1H), 7.61 (dd, J = 8.0, 2.1 Hz, 1H), 7.34 (d, J = 8.0 Hz, 1H), 2.53 (s, 3H). LCMS (Analytical Method A): Rt = 0.79 mins; MS (ESIpos) m/z = 301.1 (M + H)⁺. |

### Intermediate 162A: 2-Fluoro-3′,4′-dimethoxy-6-(1H-tetrazol-5-yl)biphenyl-4-amine

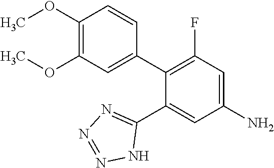 [SMILES: COc1ccc(-c2c(F)cc(N)cc2-c2nnn[nH]2)cc1OC]

To a de-gassed suspension of 5-(6-fluoro-3′,4′-dimethoxy-4-nitrobiphenyl-2-yl)-1H-tetrazole (1.3 g, 3.7 mmol) in EtOH (100 mL) was added Pd/C (5%, 200 mg, 0.094 mmol). The mixture was stirred at room temperature under an atmosphere of hydrogen for a total of 16 hours. The catalyst was removed by filtration through Celite and washed with EtOH (50 mL). The filtrate was concentrated in vacuo to afford 1.15 g (97% yield) of the title compound as yellow solid.

¹H NMR (500 MHz, DMSO-d6) δ[ppm] 6.83 (d, J=8.2 Hz, 1H), 6.65-6.58 (m, 2H), 6.55-6.48 (m, 2H), 5.79 (s, 2H), 3.72 (s, 3H), 3.57 (s, 3H).

LCMS (Analytical Method A): Rt=0.92 mins; MS (ESipos) m/z=316.0 (M+H)⁺.

In analogy to the procedure described for Intermediate 162A, the following intermediates were prepared using the corresponding nitro compound as starting material.

| Int. | Structure | Name | Analytical Data |
|---|---|---|---|
| 163A | 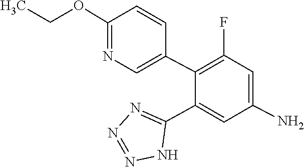 [SMILES: CCOc1ccc(-c2c(F)cc(N)cc2-c2nnn[nH]2)cn1] | 4-(6- ethoxypyridin- 3-yl)-3-fluoro- 5-(1H-tetrazol- 5-yl)aniline | ¹H NMR (500 MHz, DMSO-d6) δ [ppm] 7.80-7.73 (m, 1H), 7.30 (dd, J = 8.5, 2.2 Hz, 1H), 6.72- 6.67 (m, 2H), 6.63 (dd, J = 12.3, 2.1 Hz, 1H), 5.91 (s, 2H), 4.26 (q, J = 7.0 Hz, 2H), 1.29 (t, J = 7.0 Hz, 3H). LCMS (Analytical Method A): Rt = 0.99 mins; MS (ESIpos) m/z = 301 (M + H)⁺. |
| 164A | 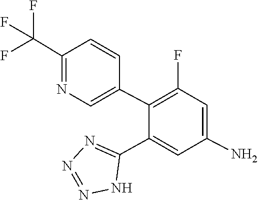 [SMILES: Nc1cc(F)c(-c2ccc(C(F)(F)F)nc2)c(-c2nnn[nH]2)c1] | 3-fluoro-5-(1H- tetrazol-5-yl)- 4-[6- (trifluoromethyl) pyridin-3- yl]aniline | ¹H NMR (500 MHz, DMSO-d6) δ [ppm] 8.43 (s, 1H), 7.83 (d, J = 8.1 Hz, 1H), 7.73 (d, J = 8.0 Hz, 1H), 6.85 (s, 1H), 6.68 (dd, J = 12.6, 2.1 Hz, 1H), 6.10 (s, 2H). LCMS (Analytical Method F): Rt = 2.44 mins; MS (ESIpos) m/z = 325.1 (M + H)⁺. |
| 165A | 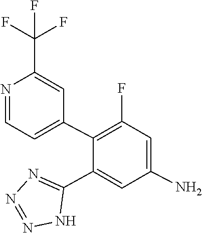 [SMILES: Nc1cc(F)c(-c2ccnc(C(F)(F)F)c2)c(-c2nnn[nH]2)c1] | 3-fluoro-5-(1H- tetrazol-5-yl)- 4-[2- (trifluoromethyl) pyridin-4- yl]aniline | ¹H NMR (500 MHz, DMSO-d6) δ 8.63 (d, J = 5.0 Hz, 1H), 7.48 (s, 1H), 7.31 (d, J = 5.0 Hz, 1H), 6.81 (s, 1H), 6.67 (dd, J = 12.9, 2.1 Hz, 1H), 6.16 (s, 2H); LCMS (Analytical Method F): Rt = 2.36 min, MS (ESIpos) m/z = 325.1 (M + H)⁺. |
| 166A | 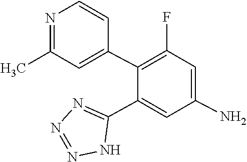 [SMILES: Cc1cc(-c2c(F)cc(N)cc2-c2nnn[nH]2)ccn1] | 3-fluoro-4-(2- methylpyridin- 4-yl)-5-(1H- tetrazol-5- yl)aniline | ¹H NMR (500 MHz, DMSO-d6) δ [ppm] 8.26 (d, J = 5.1 Hz, 1H), 6.90 (s, 1H), 6.77-6.69 (m, 2H), 6.59 (dd, J = 12.8, 2.2 Hz, 1H), 5.92 (s, 3H), 2.37 (s, 3H). LCMS (Analytical Method E): Rt = 2.17 mins; MS (ESIpos) m/z = 271.2 (M + H)⁺. |
| 167A | 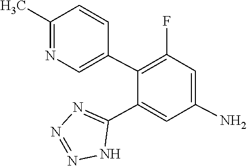 [SMILES: Cc1ccc(-c2c(F)cc(N)cc2-c2nnn[nH]2)cn1] | 3-fluoro-4-(6- methylpyridin- 3-yl)-5-(1H- tetrazol-5- yl)aniline | ¹H NMR (500 MHz, DMSO-d6) δ [ppm] 8.07 (s, 1H), 7.29 (dd, J = 8.0, 1.9 Hz, 1H), 7.15 (d, J = 8.0 Hz, 1H), 6.73 (d, J = 2.1 Hz, 1H), 6.64 (dd, J = 12.5, 2.2 Hz, 1H), 5.93 (s, 2H), 2.43 (s, 3H). LCMS (Analytical Method F): Rt = 0.90 mins; MS (ESIpos) m/z = 271.1 (M + H)⁺. |

### Intermediate 168A: 2-bromo-3-chloro-5-nitrobenzonitrile

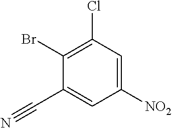 [SMILES: N#Cc1cc([N+](=O)[O-])cc(Cl)c1Br]

To 2-Amino-3-chloro-5-nitrobenzonitrile (5.0 g, 25.3 mmol) and Copper (II) bromide (9.0 g, 38.0 mmol) in acetonitrile (200 mL) was added isopentyl nitrate (5.81 mL, 38.0 mmol). The reaction mixture was heated at 65° C. for two hours. After this time the reaction was allowed to cool to room temperature. 2N HCl solution (50 mL) was added to the reaction mixture and the organics were extracted with DCM (3×40 mL). The organics were then dried over MgSO₄, filtered and concentrated under reduced pressure. The crude material was purified by Biotage Isolera™ chromatography (silica gel, eluting with heptanes-EtOAc, 1:0 to 0:1) to afford 6.3 g (83% yield) of the title compound as a yellow solid.

¹H NMR (250 MHz, DMSO-d6) δ[ppm] 8.74 (d, J=2.1 Hz, 1H), 8.81 (d, J=2.1 Hz, 1H).

LCMS (Analytical Method A): Rt=1.20 mins, no ionisation.

### Intermediate 169A: 3-chloro-2-(6-ethoxypyridin-3-yl)-5-nitrobenzonitrile

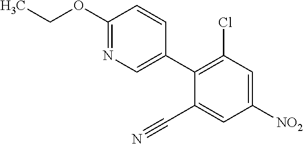 [SMILES: CCOc1ccc(-c2c(Cl)cc([N+](=O)[O-])cc2C#N)cn1]

2-Bromo-3-chloro-5-nitrobenzonitrile (3.84 g, 14.7 mmol), (6-ethoxypyridin-3-yl)boronic acid (2.33 g, 14.0 mmol) and potassium carbonate (3.86 g, 28.0 mmol) were dissolved in DME (60 mL) and water (30 mL). The reaction mixture was degassed under a stream of nitrogen before bis(triphenylphosphine)palladium(II) dichloride (490 mg, 0.70 mmol) was added. The reaction mixture was stirred at 100° C. for 2 hours. The reaction was allowed to cool to room temperature and water (50 mL) was added. The organics were extracted using DCM (3×8 mL) and the combined organics were dried over MgSO₄, filtered and concentrated under reduced pressure. The crude material was purified by Biotage Isolera™ chromatography (silica gel, eluting with heptanes-EtOAc, 1:0 to 0:1) to afford 3.5 g (56% yield) as a yellow oil.

¹H NMR (250 MHz, DMSO-d6) δ 8.88 (d, J=2.3 Hz, 1H), 8.78-8.75 (m, 1H), 8.32 (dd, J=2.5, 0.6 Hz, 1H), 7.90 (dd, J=8.6, 2.5 Hz, 1H), 7.02 (dd, J=8.6, 0.6 Hz, 1H), 4.41 (q, J=7.0 Hz, 2H), 1.37 (t, J=7.1 Hz, 3H).

LCMS (Analytical Method A): Rt=1.28 mins; MS (ESipos) m/z=303.9 (M+H)⁺.

### Intermediate 170A: 5-[2-chloro-4-nitro-6-(1H-tetrazol-5-yl)phenyl]-2-ethoxypyridine

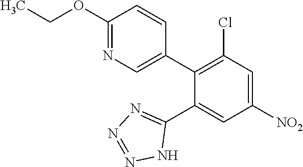 [SMILES: CCOc1ccc(-c2c(Cl)cc([N+](=O)[O-])cc2-c2nnn[nH]2)cn1]

2-Chloro-4-fluoro-5-nitrobenzonitrile (1.0 g, 2.2 mmol) and di-n-butyltin oxide (557 mg, 2.2 mmol) were dissolved in p-xylene (10 mL). The resulting mixture was heated in a sealed tube at 130° C. for an hour. After this time TMS-Azide (0.33 mL, 2.5 mmol) was added and the reaction was stirred for a further 3 hours. After this time LCMS analysis (Analytical Method A) indicated that some starting material remained and thus more TMS-Azide (0.15 mL, 1.13 mmol) was added to the reaction mixture. The reaction was stirred again for two hours at 130° C. MeOH (10 mL) was added to the reaction mixture and the solvent was then removed under reduced pressure. The crude material was purified by Biotage Isolera™ chromatography (silica gel, eluting with DCM-MeOH, 1:0 to 3:20) to afford 694 mg (63% yield) of the title compound as a light brown powder.

¹H NMR (250 MHz, DMSO-d6) δ[ppm] 8.64 (s, 2H), 7.93 (d, J=2.4 Hz, 1H), 7.53 (dd, J=8.6, 2.5 Hz, 1H), 6.83 (d, J=8.6 Hz, 1H), 4.31 (m, 2H), 1.33 (m, 3H).

LCMS (Analytical Method A): Rt=1.15 mins; MS (ESipos) m/z=346.9 (M+H)⁺.

### Intermediate 171A: 3-chloro-4-(6-ethoxypyridin-3-yl)-5-(1H-tetrazol-5-yl)aniline

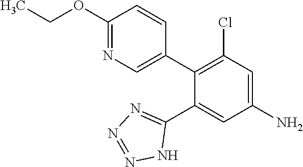 [SMILES: CCOc1ccc(-c2c(Cl)cc(N)cc2-c2nnn[nH]2)cn1]

To a de-gassed solution of 5-[2-chloro-4-nitro-6-(1H-tetrazol-5-yl)phenyl]-2-ethoxypyridine (71%, 200 mg, 0.41 mmol) in EtOH (1 mL) at room temperature was added Pd/C (10%, 9 mg, 0.01 mmol). The reaction mixture was stirred under an atmosphere of hydrogen for 24h. The reaction mixture filtered over a Celite pad, washing with methanol (3×20 mL). The filtrate was then concentrated under reduced pressure to afford 180 mg (94% yield) of the title compound as a light brown solid.

¹H NMR (250 MHz, DMSO-d6) δ[ppm] 1.27-1.32 (m, 3H), 4.21-4.29 (m, 2H), 6.67 (d, J=8.6 Hz, 1H), 6.82 (d, J=2.3 Hz, 1H), 6.91 (d, J=2.2 Hz, 1H), 7.32 (dd, J=8.5, 2.5 Hz, 1H), 7.74 (d, J=2.1 Hz, 1H).

LCMS (Analytical Method A): Rt=1.02 mins; MS (ESipos) m/z=317.0 (M+H)⁺.

### Intermediate 172A: 2-(6-ethoxypyridin-3-yl)-4-hydroxy-5-nitrobenzonitrile

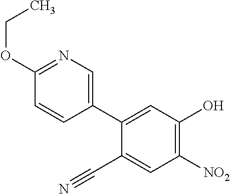 [SMILES: CCOc1ccc(-c2cc(O)c([N+](=O)[O-])cc2C#N)cn1]

Water (37 mL) and 1,2-dimethoxyethane (74 mL) were degassed with a stream of argon for 15 mins. Then 2-bromo-4-fluoro-5-nitrobenzonitrile (5.00 g, 20.4 mmol), 2-ethoxy-5-pyridine boronic acid (3.41 g, 20.4 mmol), potassium carbonate (9.31 g, 67.3 mmol) and dichlorobis(triphenylphosphine) palladium(II) (172 mg, 0.245 mmol) were added, and the reaction heated at 90° C. for 5 h. The mixture was cooled and put into water. Ethyl acetate was added and the layers separated. The aqueous layer was extracted 3× with EE. The combined organic layers were washed with brine, dried with sodium sulfate and concentrated under reduced pressure. The crude product was purified by chromatography (SiO₂, hexane/EE 0-100%-30% MeOH) to yield 1.63 g (25% yield) of the title compound (where the F atom is replaced by hydroxy).

¹H NMR (400 MHz, DMSO-d6) δ[ppm] 1.35 (t, 3H), 4.37 (q, 2H), 6.73 (s, 1H), 6.92 (dd, 1H), 7.87 (dd, 1H), 8.29 (s, 1H), 8.31 (d, 1H).

LCMS (method 1): Rt=1.06 min; MS (ESipos) m/z=286 (M+H)⁺.

In analogy to the procedure described for Intermediate 172A, the following intermediate was prepared using the corresponding pyridine boronic acid or pyridine boronic pinacol ester as starting material.

| Int. | Structure | Name | Analytical Data |
|---|---|---|---|
| 173A | 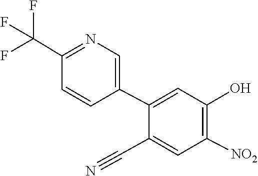 [SMILES: N#Cc1cc([N+](=O)[O-])c(O)cc1-c1ccc(C(F)(F)F)nc1] | 4-hydroxy-5- nitro-2-[6- (trifluoromethyl) pyridin-3- yl]benzonitrile | ¹H NMR (400 MHz, DMSO-d6) δ [ppm] 6.72 (s, 1H), 8.05 (d, 1H), 8.26 (dd, 1H), 8.29 (s, 1H), 8.91 (d, 1H); LCMS (method 1): Rₜ = 1.01 min; MS (ESIpos) m/z = 310 (M + H)⁺. |

### Intermediate 174A: 5-(6-ethoxypyridin-3-yl)-2-nitro-4-(1H-tetrazol-5-yl)phenol

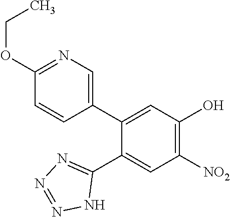 [SMILES: CCOc1ccc(-c2cc(O)c([N+](=O)[O-])cc2-c2nnn[nH]2)cn1]

The compound was prepared in analogy to Intermediate 137A.

850 mg (45% yield) were obtained as yellow solid.

1H NMR (400 MHz, DMSO-d6) δ[ppm] 1.32 (t, 3H), 4.31 (q, 2H), 6.73 (d, 1H), 7.18 (s, 1H), 7.40 (dd, 1H), 7.98 (d, 1H), 8.28 (d, 1H).

LCMS (method 1): Rt=0.85 min; MS (ESipos) m/z=329 (M+H)⁺.

In analogy to the procedure described for Intermediate 174A, the following intermediate was prepared using the corresponding nitrile as starting material.

| Int. | Structure | Name | Analytical Data |
|---|---|---|---|
| 175A | 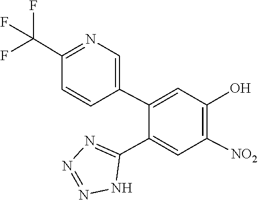 [SMILES: O=[N+]([O-])c1cc(-c2nnn[nH]2)c(-c2ccc(C(F)(F)F)nc2)cc1O] | 2-nitro-4-(1H- tetrazol-5-yl)-5- [6- (trifluoromethyl) pyridin-3- yl]phenol | ¹H NMR (400 MHz, DMSO-d6) δ [ppm] 7.17 (s, 1H), 7.90 (m, 2H), 8.40 (s, 1H), 8.57 (s, 1H); LCMS (method 1): Rₜ = 0.85 min; MS (ESIpos) m/z = 353 (M + H)⁺. |

### Intermediate 176A: 2-amino-5-(6-ethoxypyridin-3-yl)-4-(1H-tetrazol-5-yl)phenol

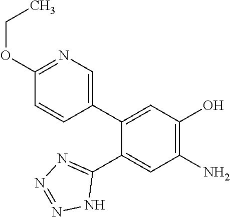 [SMILES: CCOc1ccc(-c2cc(O)c(N)cc2-c2nnn[nH]2)cn1]

The compound was prepared in analogy to Intermediate 140A.

800 mg (100% yield) were obtained as a dark foam.

¹H NMR (400 MHz, DMSO-d6) δ[ppm] 1.30 (t, 3H), 4.26 (q, 2H), 6.62 (d, 1H), 6.72 (s, 1H), 6.88 (s, 1H), 7.22 (dd, 1H), 7.78 (d, 1H).

LCMS (method 1): Rt=0.54 min; MS (ESipos) m/z=299 (M+H)⁺.

In analogy to the procedure described for Intermediate 176A, the following intermediate was prepared using the corresponding nitro compound as starting material.

| Int. | Structure | Name | Analytical Data |
|---|---|---|---|
| 177A | 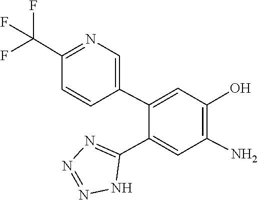 [SMILES: Nc1cc(-c2nnn[nH]2)c(-c2ccc(C(F)(F)F)nc2)cc1O] | 2-amino-4-(1H- tetrazol-5-yl)-5- [6- (trifluoromethyl) pyridin-3- yl]phenol | ¹H NMR (400 MHz, DMSO-d6) δ [ppm] 6.78 (s, 1H), 6.99 (s, 1H), 7.62 (dd, 1H), 7.74 (d, 1H), 8.35 (d, 1H). LCMS (method 1): Rₜ = 0.62 min; MS (ESIpos) m/z = 323 (M + H)⁺. |

### Intermediate 178A: 3-chloro-2-fluoro-4-[6-(trifluoromethyl)pyridin-3-yl]aniline

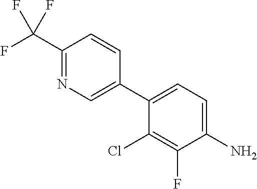 [SMILES: Nc1ccc(-c2ccc(C(F)(F)F)nc2)c(Cl)c1F]

A mixture of [6-(trifluoromethyl)pyridin-3-yl]boronic acid (1.5 g, 7.86 mmol), 4-bromo-3-chloro-2-fluoroaniline (1.18 g, 5.24 mmol), palladium(II) acetate (59 mg, 0.26 mmol), 2-dicyclohexylphosphino-2′,6′-dimethoxybiphenyl (215 mg, 0.52 mmol) and potassium phosphate (3.34 g, 15.71 mmol) in tetrahydrofuran (25 mL) in a round bottomed flask was degassed and placed under an inert nitrogen atmosphere. The reaction mixture was then stirred at 60° C. for 5 hours, where upon a LCMS analysis showed the reaction had completed. The product was then extracted in EtOAc and washed with an aqueous sodium hydrogen carbonate solution. The organic layer was then dried and filtered, with the excess solvent removed under reduced atmosphere resulting in a dark orange oil of crude product (2 g). The residual material was then dissolved in dichloromethane and purified over silica by flash column chromatography (using a gradient of eluents; EtOAc/Heptane 0-50%). The desired fractions were then combined and the excess solvent was removed under reduced pressure to afford the title compound (1.04 g, 45% yield) as orange crystals.

¹H NMR (250 MHz, DMSO-d6) δ[ppm] 5.82 (s, 2H), 6.86 (m, 1H), 7.10 (dd, J=8.4, 1.6 Hz, 1H), 7.97 (d, J=8.2 Hz, 1H), 8.13 (dd, J=7.9, 1.9 Hz, 1H), 8.80 (d, J=2.1 Hz, 1H).

LCMS (Analytical Method A): Rt=1.20 mins, MS (ESIpos): m/z=291 (M+H)⁺.

### Intermediate 179A: 3-amino-2-fluoro-6-[6-(trifluoromethyl)pyridin-3-yl]benzonitrile

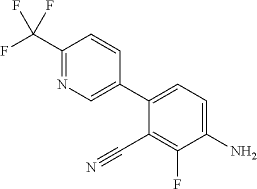 [SMILES: N#Cc1c(-c2ccc(C(F)(F)F)nc2)ccc(N)c1F]

A mixture of 3-chloro-2-fluoro-4-[6-(trifluoromethyl)pyridin-3-yl]aniline (800 mg, 2.75 mmol), potassium ferrocyanide (581 mg, 1.38 mmol), Pd₂(dba)₃ (25 mg, 0.03 mmol), dicyclohexyl[2′,4′,6′-tri(propan-2-yl)biphenyl-2-yl]phosphane (26 mg, 0.06 mmol) and potassium acetate (33.8 mg, 0.34 mmol) in 1,4-dioxane/water (6.9 mL; 1:1 v:v) was degassed via nitrogen-filled balloon for 5 minutes, prior to vial capping and heating at 100° C. for 16 hours. After this time the reaction mixture was allowed to cool to room temperature and partitioned between EtOAc and saturated aqueous sodium hydrogen carbonate solution. The biphasic mixture was filtered through a pad of Celite, washing with EtOAc; the aqueous layer was removed and the organic layer was washed with saturated aqueous sodium chloride solution, dried (MgSO₄), filtered and concentrated in vacuo. The residual material was dissolved in MeOH/DCM, adsorbed onto silica gel and purified by FCC (Biotage, 25 g), eluting with EtOAc/heptane (0-30-100%), with the desired fractions combined and concentrated in vacuo to afford the title compound as an off-white solid (0.71 g, 92% yield).

¹H NMR (250 MHz, Chloroform-d) δ 4.07 (s, 2H), 6.97-7.11 (m, 2H), 7.72 (d, J=8.1 Hz, 1H), 8.01 (dd, J=8.2, 2.2 Hz, 1H), 8.76 (d, J=2.1 Hz, 1H).

LCMS (Analytical Method A): Rt=1.12 mins, MS (ESIpos): m/z=282 (M+H)⁺.

### Intermediate 180A: 2-fluoro-3-(1H-tetrazol-5-yl)-4-[6-(trifluoromethyl)pyridin-3-yl]aniline

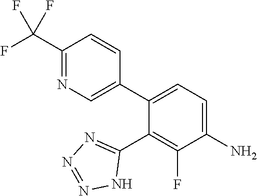 [SMILES: Nc1ccc(-c2ccc(C(F)(F)F)nc2)c(-c2nnn[nH]2)c1F]

To a suspension of 3-amino-2-fluoro-6-[6-(trifluoromethyl)pyridin-3-yl]benzonitrile (0.71 g, 2.53 mmol) and di-n-butyltinoxide (628 mg, 2.53 mmol) in p-xylene (8.4 mL) in a sealed tube was added azidotrimethylsilane (1.0 mL, 7.57 mmol). The reaction vessel was sealed and heated at 130° C. for 3 hours. After this time the reaction mixture was allowed to cool to room temperature, diluted with MeOH and concentrated in vacuo. The residual material was dissolved in MeOH/DCM, adsorbed onto silica gel and purified by flash column chromatography (Biotage, 25 g), eluting with DCM: (DCM/MeOH/Acetic acid/water [90:18:3:2 v/v]); 0->100%), with the desired fractions combined and concentrated in vacuo to afford the title compound as a pale tan solid (1.00 g, 97% yield).

¹H NMR (500 MHz, DMSO-d6) δ 8.39 (d, J=1.8 Hz, 1H), 7.77 (d, J=8.2 Hz, 1H), 7.59 (dd, J=8.1, 2.0 Hz, 1H), 7.25 (d, J=8.4 Hz, 1H), 7.11 (m, 1H), 5.87 (s, 2H).

LCMS (Analytical Method F) Rt=3.36 mins, MS (ESIpos): m/z=325 (M+H)⁺.

### Intermediate 181A: 5-Nitro-2-[4-(trifluoromethyl)-1H-pyrazol-1-yl]benzonitrile

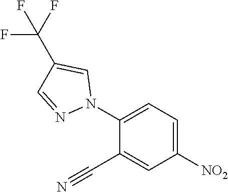 [SMILES: N#Cc1cc([N+](=O)[O-])ccc1-n1cc(C(F)(F)F)cn1]

To a solution of 2-chloro-5-nitrobenzonitrile (1.278 g, 7.0 mmol) and 4-(trifluoromethyl)-1H-pyrazole (1.0 g, 7.35 mmol) in acetonitrile (10 mL) was added potassium carbonate (2.90 g, 21 mmol) at RT. The mixture was heated to 60° C. and stirred at this temperature for 1 hour. The reaction mixture was diluted with EtOAc (50 mL), and the solids were removed by filtration. The filtrate was concentrated in vacuo and the residue was purified by Biotage Isolera™ chromatography (silica gel, eluting with heptanes-EtOAc, 1:0 to 0:1). The product containing fractions were combined and concentrated in vacuo to afford 1.77 g (89% yield) of the title compound as a yellow solid.

¹H NMR (500 MHz, DMSO-d6) δ[ppm] 9.28 (s, 1H), 8.94 (d, J=2.6 Hz, 1H), 8.68 (dd, J=9.0, 2.6 Hz, 1H), 8.47 (s, 1H), 8.19 (d, J=9.0 Hz, 1H).

LCMS (Analytical Method A): Rt=1.16 mins; MS (ESipos) m/z=283.0 (M+H).

In analogy to the procedure described for Intermediate 181A, the following intermediates were prepared:

| Int. | Structure | Name | Analytical Data |
|---|---|---|---|
| 182A | 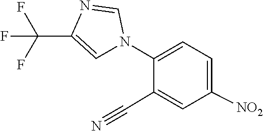 [SMILES: N#Cc1cc([N+](=O)[O-])ccc1-n1cnc(C(F)(F)F)c1] | 5-Nitro-2-[4- (trifluoromethyl)- 1H-imidazol-1- yl]benzonitrile | ¹H NMR (500 MHz, DMSO-d6) δ [ppm] 9.04 (d, J = 2.6 Hz, 1H), 8.70 (dd, J = 8.9, 2.6 Hz, 1H), 8.52-8.49 (m, 1H), 8.47 (s, 1H), 8.10 (d, J = 8.9 Hz, 1H). LCMS (Analytical Method A): Rt = 1.06 mins; MS (ESIpos) m/z = 283 (M + H)⁺. |
| 183A | 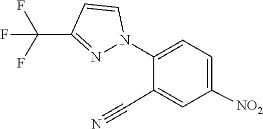 [SMILES: N#Cc1cc([N+](=O)[O-])ccc1-n1ccc(C(F)(F)F)n1] | 5-Nitro-2-[3- (trifluoromethyl)- 1H-pyrazol-1- yl]benzonitrile | ¹H NMR (500 MHz, DMSO-d6) δ [ppm] 8.96 (d, J = 2.6 Hz, 1H), 8.84-8.77 (m, 1H), 8.68 (dd, J = 9.0, 2.6 Hz, 1H), 8.18 (d, J = 9.0 Hz, 1H), 7.23 (d, J = 2.7 Hz, 1H). LCMS (Analytical Method A): Rt = 1.16 mins. |
| 184A | 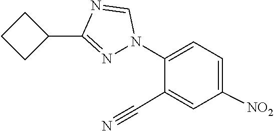 [SMILES: N#Cc1cc([N+](=O)[O-])ccc1-n1cnc(C2CCC2)n1] | 2-(3-cyclobutyl- 1H-1,2,4- triazol-1-yl)-5- nitrobenzonitrile | ¹H NMR (400 MHz, DMSO-d6) δ [ppm] = 1.91-2.11 (m, 2H), 3.20- 2.37 (m, 4H), 3.64-3.72 (m, 1H), 8.18 (d, 1H), 8.68 (dd, 1H), 8.94 (d, 1H), 9.28 (s, 1H). LCMS (method 1): Rt = 0.98 min; MS (ESIpos) m/z = 270 (M + H)⁺. |
| 185A | 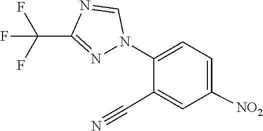 [SMILES: N#Cc1cc([N+](=O)[O-])ccc1-n1cnc(C(F)(F)F)n1] | 5-nitro-2-[3- (trifluoromethyl)- 1H-1,2,4- triazol-1- yl]benzonitrile | ¹H NMR (400 MHz, DMSO-d6) δ [ppm] = 8.27 (d, 1H), 8.77 (dd, 1H), 9.04 (d, 1H), 9.63 (s, 1H). LCMS (method 3): Rt = 1.05 min; MS (ESIpos) m/z = 284 (M + H)⁺. |
| 186A | 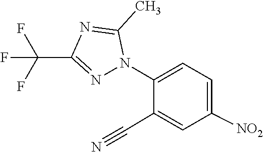 [SMILES: Cc1nc(C(F)(F)F)nn1-c1ccc([N+](=O)[O-])cc1C#N] | 2-[5-methyl-3- (trifluoromethyl)- 1H-1,2,4- triazol-1-yl]-5- nitrobenzonitrile | ¹H NMR (400 MHz, DMSO-d6) δ [ppm] = 2.55 (s, 3H), 8.27 (d, 1H), 8.77 (dd, 1H), 9.08 (d, 1H). LCMS (method 1): Rt = 1.00 min; MS (ESIpos) m/z = 298 (M + H)⁺. |
| 187A | 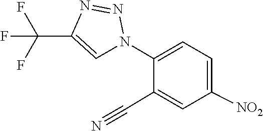 [SMILES: N#Cc1cc([N+](=O)[O-])ccc1-n1cc(C(F)(F)F)nn1] | 5-nitro-2-[4- (trifluoromethyl)- 1H-1,2,3- triazol-1- yl]benzonitrile | ¹H NMR (400 MHz, DMSO-d6) δ [ppm] = 8.27 (d, 1H), 8.80 (dd, 1H), 9.10 (d, 1H), 9.27 (s, 1H). LCMS (method 3): Rt = 1.06 min; MS (ESIpos) m/z = 284 (M + H)⁺. |
| 188A | 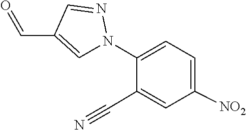 [SMILES: N#Cc1cc([N+](=O)[O-])ccc1-n1cc(C=O)cn1] | 2-(4-Formyl-1H- pyrazol-1-yl)-5- nitrobenzonitrile | ¹H NMR (500 MHz, DMSO-d6) δ [ppm] 9.99 (s, 1H), 9.31 (s, 1H), 8.94 (d, J = 2.4 Hz, 1H), 8.68 (dd, J = 9.0, 2.3 Hz, 1H), 8.47 (s, 1H), 8.19 (d, J = 9.0 Hz, 1H). LCMS (Analytical Method A): Rt = 0.95 mins; MS (ESIpos) m/z = 242.9 (M + H)⁺. |
| 189A | 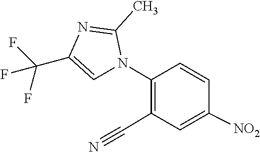 [SMILES: Cc1nc(C(F)(F)F)cn1-c1ccc([N+](=O)[O-])cc1C#N] | 2-[2-methyl-4- (trifluoromethyl)- 1H-imidazol-1- yl]-5- nitrobenzonitrile | ¹H NMR (500 MHz, DMSO-d6) δ [ppm] 9.06 (d, J = 2.6 Hz, 1H), 8.71 (dd, J = 8.8, 2.6 Hz, 1H), 8.28-8.21 (m, 1H), 8.13 (d, J = 8.8 Hz, 1H), 2.29 (s, 3H). LCMS (Analytical Method A): Rt = 1.07 mins; MS (ESIpos) m/z = 297 (M + H)⁺. |
| 190A | 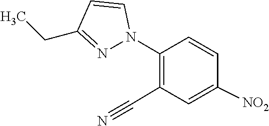 [SMILES: CCc1ccn(-c2ccc([N+](=O)[O-])cc2C#N)n1] | 2-(3-ethyl-1H- pyrazol-1-yl)-5- nitrobenzonitrile | ¹H NMR (500 MHz, DMSO-d6) δ [ppm] 8.80 (d, J = 2.6 Hz, 1H), 8.57 (dd, J = 9.2, 2.7 Hz, 1H), 8.54 (d, J = 2.6 Hz, 1H), 8.09 (d, J = 9.2 Hz, 1H), 6.60 (d, J =2.6 Hz, 1H), 2.70 (q, J = 7.6 Hz, 2H), 1.26 (t, J = 7.6 Hz, 3H). LCMS (Analytical Method A): Rt = 1.16 mins; MS (ESIpos) m/z = 243 (M + H)⁺. |
| 191A | 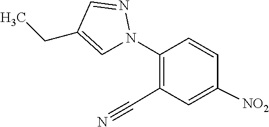 [SMILES: CCc1cnn(-c2ccc([N+](=O)[O-])cc2C#N)c1] | 2-(4-ethyl-1H- pyrazol-1-yl)-5- nitrobenzonitrile | ¹H NMR (500 MHz, DMSO-d6) δ [ppm] 8.80 (d, J = 2.6 Hz, 1H), 8.58 (dd, J = 9.1, 2.6 Hz, 1H), 8.43 (s, 1H), 8.08 (d, J = 9.2 Hz, 1H), 7.88 (s, 1H), 2.55 (q, J = 7.6 Hz, 2H), 1.22 (t, J = 7.6 Hz, 3H). LCMS (Analytical Method A): Rt = 1.15 mins; MS(ESIpos) m/z = 243 (M + H)⁺. |
| 192A | 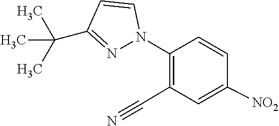 [SMILES: CC(C)(C)c1ccn(-c2ccc([N+](=O)[O-])cc2C#N)n1] | 2-(3-tert-butyl- 1H-pyrazol-1- yl)-5- nitrobenzonitrile | ¹H NMR (500 MHz, DMSO-d6) δ [ppm] 8.77 (d, J = 2.6 Hz, 1H), 8.57 (dd, J = 9.2, 2.6 Hz, 1H), 8.54 (d, J = 2.7 Hz, 1H), 8.10 (d, J = 9.2 Hz, 1H), 6.68 (d, J = 2.7 Hz, 1H), 1.33 (s, 9H). LCMS (Analytical Method A): Rt = 1.28 mins; MS (ESIpos) m/z = 271 (M + H)⁺. |
| 193A | 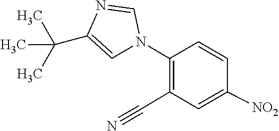 [SMILES: CC(C)(C)c1cn(-c2ccc([N+](=O)[O-])cc2C#N)cn1] | 2-(4-tert-butyl- 1H-imidazol-1- yl)-5- nitrobenzonitrile | ¹H NMR (250 MHz, DMSO-d6) δ [ppm] 8.93 (d, J = 2.6 Hz, 1H), 8.61 (dd, J = 9.0, 2.7 Hz, 1H), 8.18 (d, J = 1.4 Hz, 1H), 7.97 (d, J = 9.0 Hz, 1H), 7.48 (d, J = 1.4 Hz, 1H), 1.28 (s, 9H). LCMS (Analytical Method A): Rt = 0.96 mins; MS (ESIpos) m/z = 271 (M + H)⁺. |
| 194A | 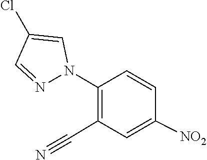 [SMILES: N#Cc1cc([N+](=O)[O-])ccc1-n1cc(Cl)cn1] | 2-(4-chloro-1H- pyrazol-1-yl)-5- nitrobenzonitrile | ¹H NMR (500 MHz, DMSO-d6) δ [ppm] 8.91 (s, 1H), 8.89 (d, J = 2.6 Hz, 1H), 8.64 (dd, J = 9.1, 2.6 Hz, 1H), 8.16 (s, 1H), 8.10 (d, J = 9.1 Hz, 1H). LCMS (Analytical Method A): Rt = 1.12 mins; MS (ESIpos) m/z = 249 (M + H)⁺. |
| 195A | 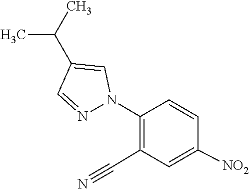 [SMILES: CC(C)c1cnn(-c2ccc([N+](=O)[O-])cc2C#N)c1] | 2-(4-isopropyl- 1H-pyrazol-1- yl)-5- nitrobenzonitrile | ¹H NMR (250 MHz, DMSO-d6) δ [ppm] 1.25 (d, J = 6.9 Hz, 6H), 2.78-3.01 (m, 1H), 7.93 (s, 1H), 8.10 (d, J = 9.2 Hz, 1H), 8.44 (s, 1H), 8.58 (dd, J = 9.1, 2.6 Hz, 1H), 8.80 (d, J = 2.6 Hz, 1H). LCMS (Analytical Method A): Rt = 1.25 mins; MS (ESIpos) m/z = 257 (M + H)⁺. |
| 196A | 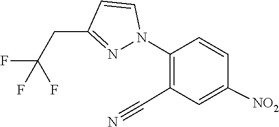 [SMILES: N#Cc1cc([N+](=O)[O-])ccc1-n1ccc(CC(F)(F)F)n1] | 5-nitro-2-[3- (2,2,2- trifluoroethyl)- 1H-pyrazol-1- yl]benzonitrile | ¹H NMR (500 MHz, Chloroform-d) δ [ppm] 8.64 (d, J = 2.6 Hz, 1H), 8.51 (dd, J = 9.1, 2.6 Hz, 1H), 8.37 (d, J = 2.7 Hz, 1H), 8.10 (d, J = 9.1 Hz, 1H), 6.64 (d, J = 2.6 Hz, 1H), 3.59 (q, J = 10.6 Hz, 2H). LCMS (Analytical Method A): Rt = 1.20 mins; MS (ESIpos) m/z = 297 (M + H)⁺. |
| 197A | 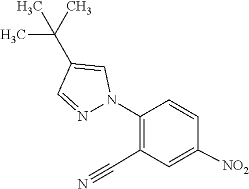 [SMILES: CC(C)(C)c1cnn(-c2ccc([N+](=O)[O-])cc2C#N)c1] | 2-(4-tert-butyl- 1H-pyrazol-1- yl)-5- nitrobenzonitrile | ¹H NMR (500 MHz, DMSO-d6) δ [ppm] 8.80 (d, J = 2.6 Hz, 1H), 8.58 (dd, J = 9.2, 2.6 Hz, 1H), 8.46 (d, J = 0.6 Hz, 1H), 8.12 (d, J = 9.2 Hz, 1H), 7.99 (d, J = 0.5 Hz, 1H), 1.30 (s, 9H). LCMS (Analytical Method A): Rt = 1.26 mins; MS (ESIpos) m/z = 271 [M + H]⁺. |

### Intermediate 198A: 2-[4-(Difluoromethyl)-1H-pyrazol-1-yl]-5-nitrobenzonitrile

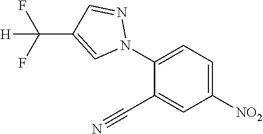 [SMILES: N#Cc1cc([N+](=O)[O-])ccc1-n1cc(C(F)F)cn1]

Two pressure-tubes (ACE) were each loaded with a 2.7M solution of [bis(2-methoxyethyl)amino]sulfur trifluoride (Deoxy Fluor) in toluene (5 mL, 13.5 mmol) and 2-(4-formyl-1H-pyrazol-1-yl)-5-nitrobenzonitrile (1.3 g, 4.75 mmol) at RT. The pressure tubes were sealed and heated with stirring at 80° C. for 5 hours. After cooling to RT, the reaction mixtures were combined and diluted with EtOAc (100 mL), washed with aq. 2M K₂CO₃ solution (2×50 mL) and brine (30 mL), dried (Na₂SO₄), filtered and concentrated at reduced pressure. The residue was purified by Biotage Isolera™ chromatography (silica gel, eluting with heptanes-EtOAc, 1:0 to 1:1). The product containing fractions were combined and concentrated in vacuo to afford 2.12 g (84% yield) of the title compound as a pale yellow crystalline solid.

¹H NMR (500 MHz, DMSO-d6) δ[ppm] 8.97-8.94 (m, 1H), 8.91 (d, J=2.6 Hz, 1H), 8.65 (dd, J=9.1, 2.6 Hz, 1H), 8.25 (s, 1H), 8.15 (d, J=9.1 Hz, 1H), 7.20 (t, J=55.5 Hz, 1H).

LCMS (Analytical Method A): Rt=1.11 mins; MS (ESIpos) m/z=264.9 (M+H)⁺.

### Intermediate 199A: 2-(3-ethyl-2-oxopyrrolidin-1-yl)-5-nitrobenzonitrile

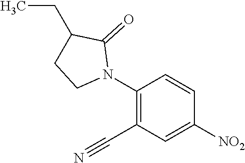 [SMILES: CCC1CCN(c2ccc([N+](=O)[O-])cc2C#N)C1=O]

To a solution of 3-ethylpyrrolidin-2-one (0.89 g, 7.87 mmol) in DMF (25 mL) was added sodium hydride (60% suspension in mineral oil, 346 mg, 8.65 mmol) at 0° C. (ice-bath). The mixture was stirred for 30 minutes at 0° C., then the ice-bath was removed and the mixture stirred for another 30 minutes at room temperature. A solution of 2-fluoro-5-nitrobenzonitrile (1.31 g, 7.87 mmol) in DMF (5 ml) was added dropwise via syringe and the resulting dark red solution was stirred for 1 hour at room temperature. The reaction mixture was poured into a mixture of 2M HCl (50 mL) and crushed ice (˜50 g) and the organics were extracted with EtOAc (3×50 mL). The combined organic extracts were washed with water (80 mL) and brine (50 mL), dried (Na₂SO₄) and concentrated at reduced pressure. The residue was purified was purified by Biotage Isolera™ chromatography [SNAP Cartridge KP-Sil 50 g; 0-50% EtOAc in heptane, 16 column volumes]. The product containing fractions were combined and concentrated in vacuo to afford the title compound (1.18 g, 57% yield) as pale yellow solid.

¹H NMR (500 MHz, DMSO-d6) δ[ppm] 8.74 (d, J=2.7 Hz, 1H), 8.54 (dd, J=9.0, 2.7 Hz, 1H), 7.79 (d, J=9.0 Hz, 1H), 4.05-3.97 (m, 1H), 3.87-3.80 (m, 1H), 2.66-2.57 (m, 1H), 2.37-2.28 (m, 1H), 1.91-1.73 (m, 2H), 1.58-1.46 (m, 1H), 0.97 (t, J=7.5 Hz, 3H).

LCMS (Analytical Method A): Rt=1.06 mins; MS (ESipos) m/z=260 (M+H)⁺.

### Intermediate 200A: 5-{5-Nitro-2-[4-(trifluoromethyl)-1H-pyrazol-1-yl]phenyl}-1H-tetrazole

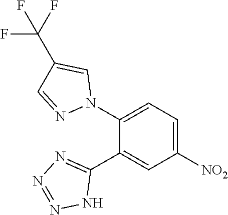 [SMILES: O=[N+]([O-])c1ccc(-n2cc(C(F)(F)F)cn2)c(-c2nnn[nH]2)c1]

A pressure tube (ACE) was loaded with 5-nitro-2-[4-(trifluoromethyl)-1H-pyrazol-1-yl]benzonitrile (1.77 g, 6.21 mmol), p-xylene (8 mL), di-n-butyltin oxide (1.546 g, 6.21 mmol) and azidotrimethylsilane (1.65 mL, 12.42 mmol). The pressure tube was sealed and heated with stirring at 130° C. for 1 hour. The reaction was cooled to RT, MeOH (10 mL) was added and the mixture stirred at RT for 1 hour, then concentrated at reduced pressure. The residue was purified by Biotage Isolera™ chromatography (silica gel, eluting with DCM-MeOH, 1:0 to 4:1) The product containing fractions were combined and concentrated in vacuo to afford 1.7 g (67% yield) of the title compound as a yellow oil.

¹H NMR (500 MHz, DMSO-d6) δ[ppm] 8.84 (s, 1H), 8.72 (d, J=2.6 Hz, 1H), 8.54 (dd, J=8.8, 2.6 Hz, 1H), 8.11 (s, 1H), 8.04 (d, J=8.8 Hz, 1H).

LCMS (Analytical Method A): Rt=1.03 mins; MS (ESipos) m/z=326 (M+H)⁺.

In analogy to the procedure described for Intermediate 200A, the following intermediates were prepared:

| Int. | Structure | Name | Analytical Data |
|---|---|---|---|
| 201A | 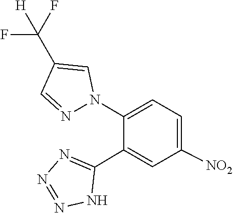 [SMILES: O=[N+]([O-])c1ccc(-n2cc(C(F)F)cn2)c(-c2nnn[nH]2)c1] | 5-{2-[4- (Difluoromethyl)- 1H-pyrazol-1- yl]-5- nitrophenyl}- 1H-tetrazole | ¹H NMR (500 MHz, Methanol-d4) δ [ppm] 8.73 (d, J = 2.6 Hz, 1H), 8.57 (dd, J = 8.8, 2.6 Hz, 1H), 8.27 (s, 1H), 7.97 (d, J = 8.8 Hz, 1H), 7.79 (s, 1H), 6.87 (t, J = 56.0 Hz, 1H). LCMS (Analytical Method A): Rt = 0.95 mins; MS (ESIpos) m/z = 308 (M + H)⁺. |
| 202A | 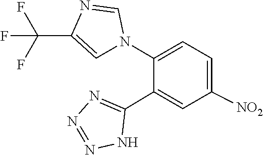 [SMILES: O=[N+]([O-])c1ccc(-n2cnc(C(F)(F)F)c2)c(-c2nnn[nH]2)c1] | 5-{5-Nitro-2-[4- (trifluoromethyl)- 1H-imidazol-1- yl]phenyl}-1H- tetrazole | ¹H NMR (500 MHz, DMSO-d6) δ [ppm] 8.86 (d, J = 2.6 Hz, 1H), 8.57 (dd, J = 8.7, 2.6 Hz, 1H), 8.10-8.06 (m, 1H), 8.04 (s, 1H), 8.02 (d, J = 8.7 Hz, 1H). LCMS (Analytical Method A): Rt = 0.96 mins; MS (ESIpos) m/z = 326.0 (M + H)⁺. |
| 203A | 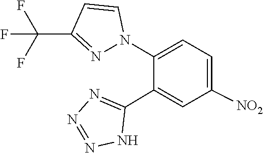 [SMILES: O=[N+]([O-])c1ccc(-n2ccc(C(F)(F)F)n2)c(-c2nnn[nH]2)c1] | 5-{5-Nitro-2-[3- (trifluoromethyl)- 1H-pyrazol-1- yl]phenyl}-1H- tetrazole | ¹H NMR (500 MHz, DMSO-d6) δ [ppm] 8.74 (d, J = 2.6 Hz, 1H), 8.60 (dd, J = 8.8, 2.6 Hz, 1H), 8.41-8.35 (m, 1H), 8.10 (d, J = 8.8 Hz, 1H), 7.01 (d, J = 2.6 Hz, 1H). LCMS (Analytical Method A): Rt = 1.04 mins; MS (ESIpos) m/z = 326.0 (M + H)⁺. |
| 204A | 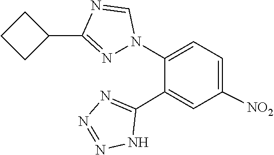 [SMILES: O=[N+]([O-])c1ccc(-n2cnc(C3CCC3)n2)c(-c2nnn[nH]2)c1] | 5-[2-(3- cyclobutyl-1H- 1,2,4-triazol-1- yl)-5- nitrophenyl]- 1H-tetrazole | ¹H NMR (400 MHz, DMSO-d6) δ [ppm] = 1.79-1.89 (m, 1H), 1.91- 2.02 (m, 1H), 2.12-2.29 (m, 4H), 3.47-3.58 (m, 1H), 8.00 (d, 1H), 8.50 (dd, 1H), 8.71 (d, 1H), 8.74 (s, 1H). LCMS (method 1): Rt = 0.78 min; MS (ESIpos) m/z = 313 (M + H)⁺. |
| 205A | 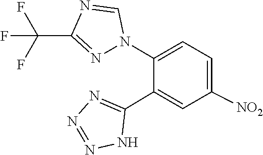 [SMILES: O=[N+]([O-])c1ccc(-n2cnc(C(F)(F)F)n2)c(-c2nnn[nH]2)c1] | 5-{5-nitro-2-[3- (trifluoromethyl)- 1H-1,2,4- triazol-1- yl]phenyl}-1H- tetrazole | LCMS (method 3): Rt = 0.86 min; MS (ESIpos) m/z = 327 (M + H)⁺. |
| 206A | 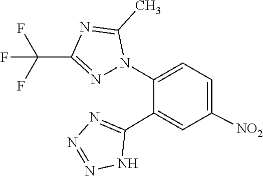 [SMILES: Cc1nc(C(F)(F)F)nn1-c1ccc([N+](=O)[O-])cc1-c1nnn[nH]1] | 5-{2-[5-methyl- 3- (trifluoromethyl)- 1H-1,2,4- triazol-1-yl]-5- nitrophenyl}- 1H-tetrazole | ¹H NMR (400 MHz, DMSO-d6) δ [ppm] = 2.25 (s, 3H), 8.12 (d, 1H), 8.54 (dd, 1H), 8.99 (d, 1H). LCMS (method 1): Rt = 0.84 min; MS (ESIpos) m/z = 341 (M + H)⁺. |
| 207A | 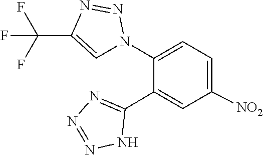 [SMILES: O=[N+]([O-])c1ccc(-n2cc(C(F)(F)F)nn2)c(-c2nnn[nH]2)c1] | 5-{5-nitro-2-[4- (trifluoromethyl)- 1H-1,2,3- triazol-1- yl]phenyl}-1H- tetrazole | LCMS (method 3): Rt = 0.86 min; MS (ESIpos) m/z = 327 (M + H)⁺. |

### Intermediate 208A: 5-amino-2-[2-methyl-4-(trifluoromethyl)-1H-imidazol-1-yl]benzonitrile

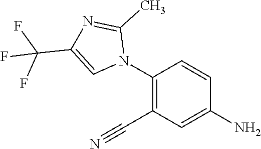 [SMILES: Cc1nc(C(F)(F)F)cn1-c1ccc(N)cc1C#N]

To a de-gassed solution of 2-[2-methyl-4-(trifluoromethyl)-1H-imidazol-1-yl]-5-nitrobenzonitrile (˜90%, 950 mg, 2.886 mmol) in EtOH (25 mL) was added Pd/C (10%, 100 mg, 0.094 mmol). The mixture was stirred at room temperature under an atmosphere of hydrogen for 2 hours. The catalyst was removed by filtration through Celite and washed with EtOH (50 mL). The filtrate was concentrated at reduced pressure and purified by Biotage Isolera™ chromatography (silica gel, eluting with heptanes-EtOAc, 1:0 to 0:1) to afford 0.62 g (72% yield) of the title compound as a yellow solid.

¹H NMR (500 MHz, DMSO-d6) δ[ppm] 7.98-7.92 (m, 1H), 7.33 (d, J=8.7 Hz, 1H), 7.02 (d, J=2.6 Hz, 1H), 6.94 (dd, J=8.7, 2.6 Hz, 1H), 6.04 (s, 2H), 2.17 (s, 3H).

LCMS (Analytical Method A): Rt=0.99 mins; MS (ESipos) m/z=267 (M+H)⁺.

In analogy to the procedure described for Intermediate 208A, the following intermediates were prepared:

| Int. | Structure | Name | Analytical Data |
|---|---|---|---|
| 209A | 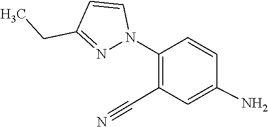 [SMILES: CCc1ccn(-c2ccc(N)cc2C#N)n1] | 5-amino-2-(3- ethyl-1H- pyrazol-1- yl)benzonitrile | ¹H NMR (500 MHz, DMSO-d6) δ [ppm] 7.96 (d, J = 2.4 Hz, 1H), 7.33 (d, J = 8.7 Hz, 1H), 6.94 (d, J = 2.5 Hz, 1H), 6.91 (dd, J = 8.7, 2.6 Hz, 1H), 6.31 (d, J = 2.3 Hz, 1H), 5.77 (s, 2H), 2.62 (q, J = 7.6 Hz, 2H), 1.21 (t, J = 7.6 Hz, 3H). LCMS (Analytical Method A): Rt = 0.99 mins; MS (ESIpos) m/z = 213 (M + H)⁺. |
| 210A | 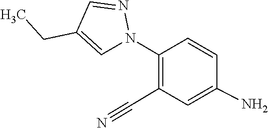 [SMILES: CCc1cnn(-c2ccc(N)cc2C#N)c1] | 5-amino-2-(4- ethyl-1H- pyrazol-1- yl)benzonitrile | ¹H NMR (500 MHz, Chloroform-d) δ [ppm] 7.69 (s, 1H), 7.57 (s, 1H), 7.43 (d, J = 8.7 Hz, 1H), 6.93 (d, J = 2.6 Hz, 1H), 6.91 (dd, J = 8.7, 2.7 Hz, 1H), 3.79 (s, 2H), 2.56 (q, J = 7.6 Hz, 2H), 1.25 (t, J = 7.6 Hz, 3H). LCMS (Analytical Method A): Rt = 1.01 mins; MS (ESIpos) m/z = 213 (M + H)⁺. |
| 211A | 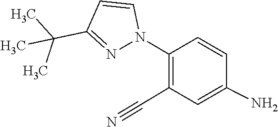 [SMILES: CC(C)(C)c1ccn(-c2ccc(N)cc2C#N)n1] | 5-amino-2-(3- tert-butyl-1H- pyrazol-1- yl)benzonitrile | ¹H NMR (500 MHz, DMSO-d6) δ [ppm] 7.95 (d, J = 2.4 Hz, 1H), 7.35 (d, J = 8.7 Hz, 1H), 6.95 (d, J = 2.5 Hz, 1H), 6.92 (dd, J = 8.7, 2.6 Hz, 1H), 6.37 (d, J = 2.4 Hz, 1H), 5.74 (s, 2H), 1.29 (s, 9H). LCMS (Analytical Method A): Rt = 1.13 mins; MS (ESIpos) m/z = 241 (M + H)⁺. |
| 212A | 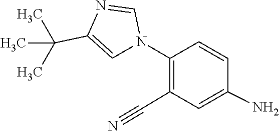 [SMILES: CC(C)(C)c1cn(-c2ccc(N)cc2C#N)cn1] | 5-amino-2-(4- tert-butyl-1H- imidazol-1- yl)benzonitrile | ¹H NMR (500 MHz, DMSO-d6) δ 7.73 (d, J = 1.3 Hz, 1H), 7.26 (d, J = 8.7 Hz, 1H), 7.07 (d, J = 1.4 Hz, 1H), 6.96 (d, J = 2.6 Hz, 1H), 6.92 (dd, J = 8.7, 2.6 Hz, 1H), 5.83 (s, 2H), 1.24 (s, 9H). LCMS (Analytical Method E): Rt = 2.40 mins; MS (ESIpos) m/z = 241 (M + H)⁺. |
| 213A | 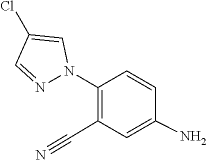 [SMILES: N#Cc1cc(N)ccc1-n1cc(Cl)cn1] | 5-amino-2-(4- chloro-1H- pyrazol-1- yl)benzonitrile | ¹H NMR (250 MHz, DMSO-d6) δ 8.39 (d, J = 0.6 Hz, 1H), 7.84 (d, J = 0.6 Hz, 1H), 7.35 (d, J = 8.7 Hz, 1H), 7.00-6.85 (m, 2H), 5.91 (s, 2H). LCMS (Analytical Method A): Rt = 1.01 mins; MS (ESIpos) m/z = 219 (M + H)⁺. |
| 214A | 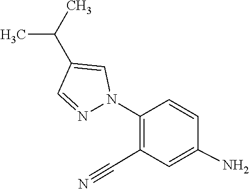 [SMILES: CC(C)c1cnn(-c2ccc(N)cc2C#N)c1] | 5-amino-2-(4- isopropyl-1H- pyrazol-1- yl)benzonitrile | ¹H NMR (250 MHz, DMSO-d6) δ 1.22 (d, J = 6.8 Hz, 6H), 2.76-2.93 (m, 1H), 5.78 (s, 2H), 6.80-7.02 (m, 2H), 7.34 (d, J = 8.6 Hz, 1H), 7.60 (s, 1H), 7.89 (s, 1H). LCMS (Analytical Method F): Rt = 3.85 mins; MS (ESIpos) m/z = 227.0 (M + H)⁺. |
| 215A | 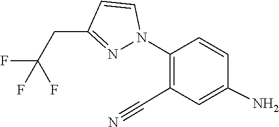 [SMILES: N#Cc1cc(N)ccc1-n1ccc(CC(F)(F)F)n1] | 5-amino-2-[3- (2,2,2- trifluoroethyl)- 1H-pyrazol-1- yl]benzonitrile | ¹H NMR (500 MHz, Chloroform-d) δ 7.87 (d, J = 2.5 Hz, 1H), 7.45 (d, J = 8.6 Hz, 1H), 6.94 (d, J = 2.6 Hz, 1H), 6.92 (dd, J = 8.6, 2.7 Hz, 1H), 6.47 (d, J = 2.3 Hz, 1H), 4.02 (s, 2H), 3.55 (q, J = 10.7 Hz, 2H); LCMS (Analytical Method A): Rt = 1.09 mins; MS (ESIpos) m/z = 267 (M + H)⁺. |
| 216A | 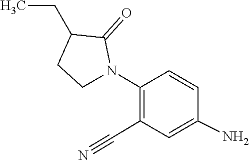 [SMILES: CCC1CCN(c2ccc(N)cc2C#N)C1=O] | 5-amino-2-(3- ethyl-2- oxopyrrolidin-1- yl)benzonitrile | ¹H NMR (500 MHz, Chloroform-d) δ 7.12-7.07 (m, 1H), 6.88-6.81 (m, 2H), 3.95 (s, 2H), 3.80-3.67 (m, 2H), 2.62-2.50 (m, 1H), 2.41- 2.29 (m, 1H), 2.02-1.85 (m, 2H), 1.64-1.48 (m, 1H), 1.03 (t, J = 7.5 Hz, 3H). LCMS (Analytical Method A): Rt = 0.91 mins; MS (ESIpos) m/z = 230 (M + H)⁺. |
| 217A | 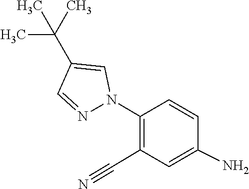 [SMILES: CC(C)(C)c1cnn(-c2ccc(N)cc2C#N)c1] | 5-amino-2-(4- tert-butyl-1H- pyrazol-1- yl)benzonitrile | ¹H NMR (500 MHz, DMSO-d6) δ 7.92- 7.86 (m, 1H), 7.67-7.60 (m, 1H), 7.35 (d, J = 8.7 Hz, 1H), 6.99- 6.88 (m, 2H), 1.26 (s, 9H). LCMS (Analytical Method A): Rt = 1.12 mins; MS (ESIpos) m/z = 241(M + H)⁺. |

### Intermediate 218A: 3-(1H-Tetrazol-5-yl)-4-[4-(trifluoromethyl)-1H-pyrazol-1-yl]aniline

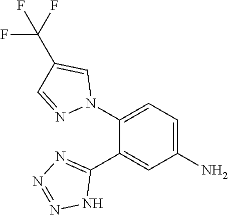 [SMILES: Nc1ccc(-n2cc(C(F)(F)F)cn2)c(-c2nnn[nH]2)c1]

To a degassed solution of 5-{5-nitro-2-[4-(trifluoromethyl)-1H-pyrazol-1-yl]phenyl}-1H-tetrazole (˜80%, 1.7 g, 4.18 mmol) in EtOH (30 mL) was added Pd/C (10%, 100 mg, 0.094 mmol). The mixture was stirred at room temperature under an atmosphere of hydrogen for 18 hours. The catalyst was removed by filtration through Celite and washed with EtOH (50 mL). The filtrate was concentrated in vacuo. The residue was purified by preparative HPLC (Method B) to afford 1.09 g (87% yield) of the title compound as white solid.

¹H NMR (500 MHz, DMSO-d6) δ[ppm] 8.52 (s, 1H), 7.86 (s, 1H), 7.32 (d, J=8.6 Hz, 1H), 6.94 (s, 1H), 6.83 (dd, J=8.6, 2.6 Hz, 1H), 5.86 (s, 2H).

LCMS (Analytical Method F): Rt=2.16 mins; MS (ESipos) m/z=296.1 (M+H)⁺.

In analogy to the procedure described for Intermediate 218A, the following intermediates were prepared:

| Int. | Structure | Name | Analytical Data |
|---|---|---|---|
| 219A | 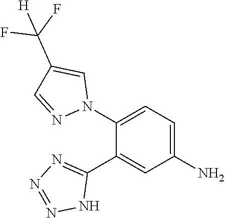 [SMILES: Nc1ccc(-n2cc(C(F)F)cn2)c(-c2nnn[nH]2)c1] | 4-[4- (Difluoromethyl)- 1H-pyrazol-1- yl]-3-(1H- tetrazol-5- yl)aniline | ¹H NMR (500 MHz, DMSO-d6) δ [ppm] 8.14 (s, 1H), 7.67 (s, 1H), 7.29 (d, J = 8.6 Hz, 1H), 7.15- 6.85 (m, 2H), 6.83 (dd, J = 8.6, 2.5 Hz, 1H), 5.80 (s, 2H); LCMS (Analytical Method F): Rt = 1.68 mins; MS (ESIpos) m/z = 278.1 (M + H)⁺. |
| 220A | 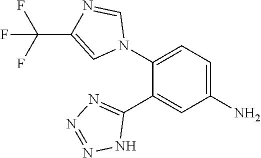 [SMILES: Nc1ccc(-n2cnc(C(F)(F)F)c2)c(-c2nnn[nH]2)c1] | 3-(1H-Tetrazol- 5-yl)-4-[4- (trifluoromethyl)- 1H-imidazol-1- yl]aniline | ¹H NMR (500 MHz, DMSO-d6) δ [ppm] 7.75 (s, 1H), 7.74 (s, 1H), 7.27 (d, J = 8.6 Hz, 1H), 7.04 (s, 1H), 6.82 (dd, J = 8.6, 2.6 Hz, 1H), 5.88 (s, 2H). LCMS (Analytical Method F): Rt = 1.86 mins; MS (ESIpos) m/z = 296.1 (M + H)⁺. |
| 221A | 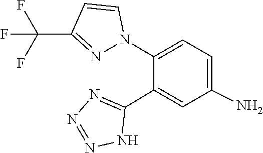 [SMILES: Nc1ccc(-n2ccc(C(F)(F)F)n2)c(-c2nnn[nH]2)c1] | 3-(1H-Tetrazol- 5-yl)-4-[3- (trifluoromethyl)- 1H-pyrazol-1- yl]aniline | ¹H NMR (500 MHz, DMSO-d6) δ [ppm] 8.02-7.94 (m, 1H), 7.34 (d, J = 8.6 Hz, 1H), 6.94 (s, 1H), 6.84 (dd, J = 8.6, 2.6 Hz, 1H), 6.78 (d, J = 2.4 Hz, 1H), 5.87 (s, 2H). LCMS (Analytical Method F): Rt = 2.14 mins; MS (ESIpos) m/z = 296.1 (M + H)⁺. |
| 222A | 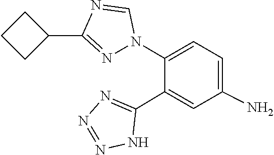 [SMILES: Nc1ccc(-n2cnc(C3CCC3)n2)c(-c2nnn[nH]2)c1] | 4-(3-cyclobutyl- 1H-1,2,4- triazol-1-yl)-3- (1H-tetrazol-5- yl)aniline | ¹H NMR (400 MHz, DMSO-d6) δ [ppm] = 1.74-1.84 (m, 1H), 1.87- 1.98 (m, 1H), 2.06-2.22 (m, 4H), 3.37-3.46 (m, 1H), 5.83 (s, br, 2H), 6.81 (dd, 1H), 6.94 (s, br, 1H), 7.29 (d, 1H), 8.44 (s, 1H). LCMS (method 1): Rt = 0.58 min; MS (ESIpos) m/z = 283 (M + H)⁺. |
| 223A | 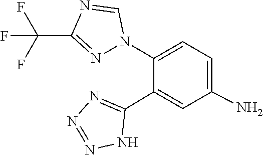 [SMILES: Nc1ccc(-n2cnc(C(F)(F)F)n2)c(-c2nnn[nH]2)c1] | 3-(1H-tetrazol- 5-yl)-4-[3- (trifluoromethyl)- 1H-1,2,4- triazol-1- yl]aniline | ¹H NMR (400 MHz, DMSO-d6) δ [ppm] = 6.64 (dd, 1H), 7.19 (d, 1H), 7.24 (d, 1H), 8.75 (s, 1H). LCMS (method 1): Rt = 0.64 min; MS (ESIpos) m/z = 297 (M + H)⁺. |
| 224A | 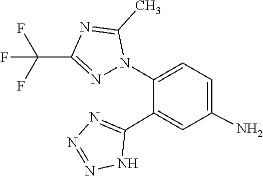 [SMILES: Cc1nc(C(F)(F)F)nn1-c1ccc(N)cc1-c1nnn[nH]1] | 4-[5-methyl-3- (trifluoromethyl)- 1H-1,2,4- triazol-1-yl]-3- (1H-tetrazol-5- yl)aniline | ¹H NMR (400 MHz, DMSO-d6) δ [ppm] = 2.19 (s, 3H), 6.75 (dd, 1H), 7.24 (d, 1H), 7.27 (d, 1H). LCMS (method 1): Rt = 0.67 min; MS (ESIpos) m/z = 311 (M + H)⁺. |
| 225A | 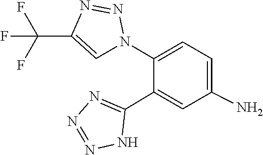 [SMILES: Nc1ccc(-n2cc(C(F)(F)F)nn2)c(-c2nnn[nH]2)c1] | 3-(1H-tetrazol- 5-yl)-4-[4- (trifluoromethyl)- 1H-1,2,3- triazol-1- yl]aniline | ¹H NMR (400 MHz, DMSO-d6) δ [ppm] = 6.69 (dd, 1H), 7.20- 7.22 (m, 2H), 8.91 (s, 1H). LCMS (method 1): Rt = 0.68 min; MS (ESIpos) m/z = 297 (M + H)⁺. |

### Intermediate 226A—4-[2-methyl-4-(trifluoromethyl)-1H-imidazol-1-yl]-3-(1H-tetrazol-5-yl)aniline

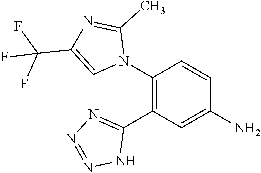 [SMILES: Cc1nc(C(F)(F)F)cn1-c1ccc(N)cc1-c1nnn[nH]1]

A pressure tube (ACE) was loaded with 5-amino-2-[2-methyl-4-(trifluoromethyl)-1H-imidazol-1-yl]benzonitrile (0.60 g, 2.07 mmol), p-xylene (5 mL), di-n-butyltin oxide (0.516 g, 2.07 mmol) and azidotrimethylsilane (413 μL, 3.11 mmol). The pressure tube was sealed and heated with stirring at 130° C. for 2 hours. The reaction was cooled to RT, MeOH (10 mL) was added and the mixture stirred at RT for 1 hour, then concentrated at reduced pressure. The crude material was purified by preparative HPLC (Method B) to afford 450 mg (52% yield) of the title compound as a white solid.

¹H NMR (500 MHz, DMSO-d6) δ[ppm] 7.55-7.47 (m, 1H), 7.40-6.88 (m, 4H), 6.73 (dd, J=8.5, 2.6 Hz, 1H), 1.96 (s, 3H).

LCMS (Analytical Method F): Rt=1.91 mins; MS (ESipos) m/z=310.2 (M+H)⁺.

In analogy to the procedure described for Intermediate 226A, the following intermediates were prepared:

| Int. | Structure | Name | Analytical Data |
|---|---|---|---|
| 227A | 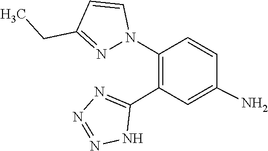 [SMILES: CCc1ccn(-c2ccc(N)cc2-c2nnn[nH]2)n1] | 4-(3-ethyl-1H- pyrazol-1-yl)-3- (1H-tetrazol-5- yl)aniline | ¹H NMR (500 MHz, DMSO-d6) δ [ppm] 7.55 (d, J = 2.3 Hz, 1H), 7.26 (d, J = 9.1 Hz, 1H), 6.85- 6.79 (m, 2H), 6.11 (d, J = 2.3 Hz, 1H), 5.68 (s, 2H), 2.42 (q, J = 7.6 Hz, 2H), 1.03 (t, J = 7.6 Hz, 3H). LCMS (Analytical Method F): Rt = 1.74 mins; MS (ESIpos) m/z = 256.1 (M + H)⁺. |
| 228A | 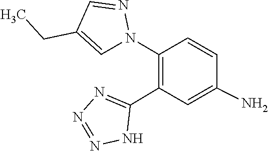 [SMILES: CCc1cnn(-c2ccc(N)cc2-c2nnn[nH]2)c1] | 4-(4-ethyl-1H- pyrazol-1-yl)-3- (1H-tetrazol-5- yl)aniline | ¹H NMR (500 MHz, DMSO-d6) δ [ppm] 7.52-7.49 (m, 1H), 7.27 (s, 1H), 7.24 (d, J = 9.1 Hz, 1H), 6.84-6.79 (m, 2H), 5.71 (s, 2H), 2.41 (q, J = 7.6 Hz, 2H), 1.12 (t, J = 7.6 Hz, 3H). LCMS (Analytical Method F): Rt = 1.88 mins; MS (ESIpos) m/z = 256.1 (M + H)⁺. |
| 229A | 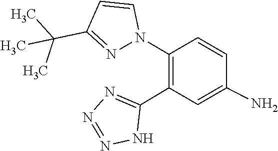 [SMILES: CC(C)(C)c1ccn(-c2ccc(N)cc2-c2nnn[nH]2)n1] | 4-(3-tert-butyl- 1H-pyrazol-1- yl)-3-(1H- tetrazol-5- yl)aniline | ¹H NMR (500 MHz, DMSO-d6)δ [ppm] 7.68 (d, J = 2.3 Hz, 1H), 7.29 (d, J = 8.6 Hz, 1H), 6.82 (dd, J = 8.6, 2.6 Hz, 1H), 6.78 (d, J = 2.5 Hz, 1H), 6.18 (d, J = 2.4 Hz, 1H), 5.65 (s, 2H), 1.04 (s, 9H). LCMS (Analytical Method F): Rt = 2.36 mins; MS (ESIpos) m/z = 284.2 (M + H)⁺. |
| 230A | 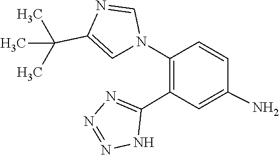 [SMILES: CC(C)(C)c1cn(-c2ccc(N)cc2-c2nnn[nH]2)cn1] | 4-(4-tert-butyl- 1H-imidazol-1- yl)-3-(1H- tetrazol-5- yl)aniline | ¹H NMR (500 MHz, DMSO-d6) δ [ppm] 8.32-8.18 (m, 1H), 7.18 (d, J = 8.5 Hz, 1H), 7.14-7.04 (m, 1H), 6.97-6.89 (m, 1H), 6.70 (dd, J = 8.5, 2.6 Hz, 1H), 1.21 (s, 9H). LCMS (Analytical Method F): Rt = 1.29 mins; MS (ESIpos) m/z = 284 (M + H)⁺. |
| 231A | 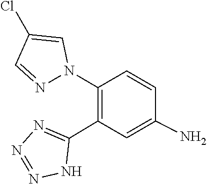 [SMILES: Nc1ccc(-n2cc(Cl)cn2)c(-c2nnn[nH]2)c1] | 4-(4-chloro-1H- pyrazol-1-yl)-3- (1H-tetrazol-5- yl)aniline | ¹H NMR (250 MHz, DMSO-d6) δ [ppm] 8.08 (d, J = 0.6 Hz, 1H), 7.53 (d, J = 0.6 Hz, 1H), 7.26 (d, J = 8.6 Hz, 1H), 6.90 (d, J = 2.5 Hz, 1H), 6.81 (dd, J = 8.6, 2.6 Hz, 1H). LCMS (Analytical Method F): Rt = 0.85 mins; MS (ESIpos) m/z = 262 (M + H)⁺. |
| 232A | 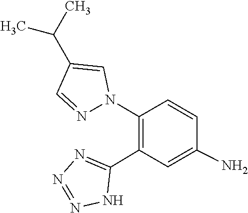 [SMILES: CC(C)c1cnn(-c2ccc(N)cc2-c2nnn[nH]2)c1] | 4-(4-Isopropyl- 1H-pyrazol-1- yl)-3-(1H- tetrazol-5- yl)aniline | ¹H NMR (250 MHz, DMSO-d6) δ [ppm] 1.15 (d, J = 6.9 Hz, 6H), 2.66-2.89 (m, 1H), 6.66-6.90 (m, 2H), 7.22-7.37 (m, 2H), 7.48 (s, 1H). LCMS (Analytical Method E): Rt = 2.40 mins; m/z (ESI) = 270.5 (M + H)⁺. |
| 233A | 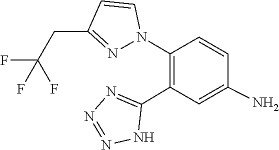 [SMILES: Nc1ccc(-n2ccc(CC(F)(F)F)n2)c(-c2nnn[nH]2)c1] | 3-(1H-Tetrazol- 5-yl)-4-[3- (2,2,2- trifluoroethyl)- 1H-pyrazol-1- yl]aniline | ¹H NMR (500 MHz, DMSO-d6) δ [ppm] 7.72 (d, J = 2.3 Hz, 1H), 7.27 (d, J = 8.6 Hz, 1H), 6.85 (s, 1H), 6.82 (dd, J = 8.5, 2.6 Hz, 1H), 6.29 (d, J = 2.2 Hz, 1H), 5.73 (s, 2H), 3.48 (q, J = 11.4 Hz, 2H). LCMS (Analytical Method F): Rt = 1.98 mins; MS (ESIpos) m/z = 310 (M + H)⁺. |
| 234A | 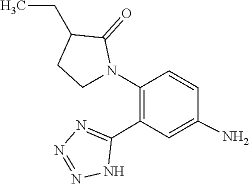 [SMILES: CCC1CCN(c2ccc(N)cc2-c2nnn[nH]2)C1=O] | 1-[4-Amino-2- (1H-tetrazol-5- yl)phenyl]-3- ethylpyrrolidin- 2-one | ¹H NMR (500 MHz, DMSO-d6) δ [ppm] 7.08 (d, J = 8.5 Hz, 1H), 6.88 (s, 1H), 6.76 (dd, J = 8.5, 2.6 Hz, 1H), 5.54 (s, 2H), 3.73- 3.62 (m, 1H), 3.61-3.52 (m, 1H), 2.25-2.13 (m, 2H), 1.82- 1.67 (m, 1H), 1.62-147 (m, 1H), 1.31-1.14 (m, 1H), 0.82 (t, J = 7.4 Hz, 3H). LCMS (Analytical Method F): Rt = 1.61 mins, MS (ESIpos) m/z = 273 (M + H)⁺. |
| 235A | 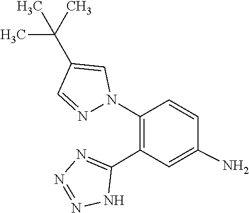 [SMILES: CC(C)(C)c1cnn(-c2ccc(N)cc2-c2nnn[nH]2)c1] | 4-(4-tert-Butyl- 1H-pyrazol-1- yl)-3-(1H- tetrazol-5- yl)aniline | ¹H NMR (500 MHz, DMSO-d6) δ [ppm] 7.45 (s, 1H), 7.34 (s, 1H), 7.28 (d, J = 8.6 Hz, 1H), 6.90- 6.68 (m, 2H), 1.18 (s, 9H). LCMS (Analytical Method F): Rt = 2.43 mins, MS (ESIpos) m/z = 284 (M + H)⁺. |

### Intermediate 236A: 2-[1-(difluoromethyl)-1H-pyrazol-4-yl]-5-nitronicotinonitrile

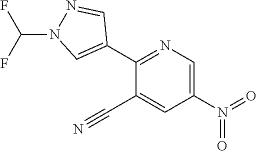 [SMILES: N#Cc1cc([N+](=O)[O-])cnc1-c1cnn(C(F)F)c1]

A mixture of 2-chloro-5-nitropyridine-3-carbonitrile (627 mg, 3.24 mmol), 1-(difluoromethyl)-4-(4,4,5,5-tetramethyl-1,3,2-dioxaborolan-2-yl)-1H-pyrazole (0.95 g, 3.894 mmol) and potassium carbonate (1.48 g, 10.71 mmol) in dimethoxyethane (8 mL) and water (5 ml) was heated at reflux under nitrogen atmosphere for 10 minutes.

Dichlorobis(triphenylphosphine)palladium(II) (30 mg, 0.043 mmol) was added to the reaction and the mixture was heated at reflux for 2 hours. After cooling to room temperature, the reaction mixture was diluted with EtOAc (80 mL) and water (30 mL). The organic layer was separated and the aqueous layer was extracted with EtOAc (50 mL). The combined organic extracts were washed with brine (30 mL), dried (Na₂SO₄) and concentrated at reduced pressure.

The residue was purified by Biotage Isolera™ chromatography [SNAP Cartridge KP-Sil 50 g; 0-50% EtOAc in heptane, 16 column volumes]. The product containing fractions were combined, concentrated in vacuo to afford the title compound (0.415 g, 48% yield) as yellow solid.

¹H NMR (500 MHz, DMSO-d6) δ[ppm] 9.58 (d, J=2.6 Hz, 1H), 9.29 (d, J=2.6 Hz, 1H), 9.13 (s, 1H), 8.57 (s, 1H), 8.02 (t, J=58.3 Hz, 1H).

LCMS (Analytical Method A): Rt=1.07 mins; m/z (ESI)=266 (M+H)⁺.

### Intermediate 237A: 2-[1-(difluoromethyl)-1H-pyrazol-4-yl]-5-nitro-3-(1H-tetrazol-5-yl)pyridine

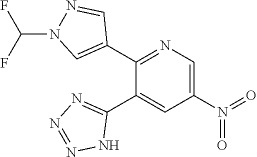 [SMILES: O=[N+]([O-])c1cnc(-c2cnn(C(F)F)c2)c(-c2nnn[nH]2)c1]

A pressure tube was loaded with 2-[1-(difluoromethyl)-1H-pyrazol-4-yl]-5-nitronicotinonitrile (0.41 g, 1.53 mmol), p-xylene (5 mL), di-n-butyltin oxide (0.381 g, 1.53 mmol) and azidotrimethylsilane (0.406 mL, 3.06 mmol). The pressure tube was sealed and heated with stirring at 130° C. for 1 hour. The reaction was then cooled to room temperature and methanol (10 mL) was added and the mixture stirred at room temperature for 1 hour, then concentrated at reduced pressure. The residue was purified by Biotage Isolera™ chromatography [SNAP Cartridge KP-Sil 25 g; 0-50% MeOH in DCM, 16 column volumes]. The product containing fractions were combined and concentrated in vacuo to afford the title compound (340 mg, 65% yield) as brown oil.

¹H NMR (500 MHz, DMSO-d6) δ[ppm] 9.53 (d, J=2.6 Hz, 1H), 8.84 (d, J=2.5 Hz, 1H), 8.39 (s, 1H), 7.84 (t, J=58.8 Hz, 1H), 7.77 (d, J=1.2 Hz, 1H) LCMS (Analytical Method A): Rt=0.89 mins; m/z (ESipos)=309 (M+H)⁺.

### Intermediate 238A: 6-[1-(difluoromethyl)-1H-pyrazol-4-yl]-5-(1H-tetrazol-5-yl)pyridin-3-amine

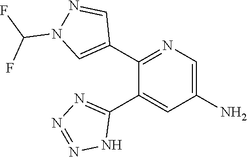 [SMILES: Nc1cnc(-c2cnn(C(F)F)c2)c(-c2nnn[nH]2)c1]

To a de-gassed solution of 2-[1-(difluoromethyl)-1H-pyrazol-4-yl]-5-nitro-3-(1H-tetrazol-5-yl)pyridine (0.330 g, 0.964 mmol) in EtOH (20 mL) was added Pd/C (10%, 100 mg, 0.094 mmol). The mixture was stirred at room temperature under an atmosphere of hydrogen for 18 hours (overnight). The catalyst was removed by filtration (Celite) and washed with EtOH (50 mL). The filtrate was concentrated in vacuo to afford the title compound (251 mg, 84% yield) as greenish-brown solid.

¹H NMR (250 MHz, Methanol-d4) δ [ppm] 8.12 (d, J=2.7 Hz, 1H), 7.75 (s, 1H), 7.40 (s, 1H), 7.38 (t, J=59.7 Hz, 1H), 7.24 (d, J=2.7 Hz, 1H).

LCMS (Analytical Method C): Rt=0.27 mins; m/z (ESipos)=279.2 (M+H)⁺.

### Intermediate 239A: 1-(3-Chlorophenyl)-N-[4-(6-{2-methyl-2-[(trimethylsilyl)oxy]propyl}pyridin-3-yl)-3-(1-{[2-(trimethylsilyl)ethoxy]methyl}-1H-tetrazol-5-yl)phenyl]cyclopropanecarboxamide

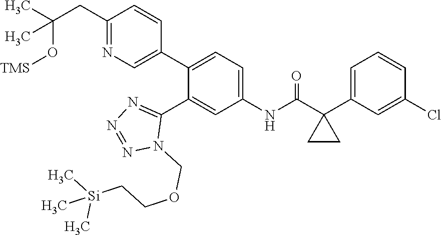 [SMILES: CC(C)(Cc1ccc(-c2ccc(NC(=O)C3(c4cccc(Cl)c4)CC3)cc2-c2nnnn2COCC[Si](C)(C)C)cn1)O[Si](C)(C)C]

Intermediate 111A (110 mg, 0.2 mmol; as a 1:1 mixture of SEM protected regioisomers), (6-{2-methyl-2-[(trimethylsilyl)oxy]propyl}pyridin-3-yl)boronic acid (107 mg, 0.4 mmol) and potassium carbonate (83 mg, 0.6 mmol) were stirred in 1,2-dimethoxyethane (4 mL) and water (2 mL) and degassed for 5 mins. Dichlorobis(triphenylphosphine)palladium(II) (14 mg, 0.02 mmol) was then added and the resulting mixture was heated at 100° C. for 1 hour giving a yellow solution. The organics were diluted with EtOAc (50 mL) and then washed with brine (2×25 mL), dried (Na₂SO₄), filtered and concentrated. The residue was purified via flash chromatography (SiO₂, EtOAc/heptane 10-50%) to afford 110 mg (79% yield) of the title compound as a colourless oil.

NMR showed a 1:1 mixture of SEM protected regioisomers.

¹H NMR (250 MHz, Chloroform-d) 6 [ppm] 8.32-8.14 (m, 1H), 7.87-7.70 (m, 1H), 7.63-7.01 (m, 9H), 5.74-4.92 (m, 2H), 3.63-3.20 (m, 2H), 2.91-2.73 (m, 2H), 1.75-1.60 (m, 2H), 1.28-1.05 (m, 8H), 0.86-0.55 (m, 2H), 0.01-0.05 (m, 9H), −0.08-0.17 (m, 9H).

LCMS (Analytical Method A): Rt=1.42 mins; MS (ESipos) m/z=619 (M-TMS+H)⁺.

### Intermediate 240A: 5-Amino-2-(3,6-dihydro-2H-pyran-4-yl)benzonitrile

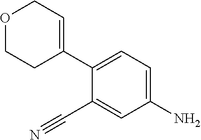 [SMILES: N#Cc1cc(N)ccc1C1=CCOCC1]

5-Amino-2-bromobenzonitrile (500 mg, 2.54 mmol), 4-(4,4,5,5-tetramethyl-1,3,2-dioxaborolan-2-yl)-3,6-dihydro-2H-pyran (559 mg, 2.67 mmol) and potassium carbonate (701 mg, 5.08 mmol) were dissolved in DME (10 mL) and water (5 mL). The reaction mixture was degassed under a stream of nitrogen before bis(triphenylphosphine)palladium(II) dichloride (89 mg, 0.13 mmol) was added. The reaction mixture was stirred at 100 C for 2 hours. After this time the reaction was complete and the reaction mixture was cooled to room temperature. Water (15 mL) was added to the reaction mixture and this was extracted with DCM (3×10 mL). The combined organics were dried (MgSO₄), filtered and concentrated under reduced pressure. The crude material was purified by flash chromatography (SiO₂, EtOAc/heptane 0-100%) to afford 270 mg (50% yield) of the title compound.

¹H NMR (250 MHz, DMSO-d6) δ[ppm] 2.29-2.40 (m, 2H), 3.79 (t, J=5.4 Hz, 2H), 4.18 (q, J=2.8 Hz, 2H), 5.59 (s, 2H), 5.82-5.96 (m, 1H), 6.71-6.90 (m, 2H), 7.14 (d, J=8.3 Hz, 1H).

LCMS (Analytical Method A): Rt=0.92 mins; MS (ESipos) m/z=201 (M+H)⁺.

### Intermediate 241A: 5-Amino-2-(tetrahydro-2H-pyran-4-yl)benzonitrile

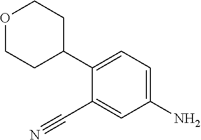 [SMILES: N#Cc1cc(N)ccc1C1CCOCC1]

5-Amino-2-(3,6-dihydro-2H-pyran-4-yl)benzonitrile (270 mg, 1.32 mmol) was dissolved in ethanol (20 mL) at room temperature. Palladium on carbon (10%, 30 mg, 26.4 mmol) was added, and the reaction stirred under an atmosphere of hydrogen at room temperature for 17 hours. Further Palladium on carbon (10%, 30 mg, 26.4 mmol) was added, and the reaction stirred for another 6h. The reaction mixture was filtered under vacuum over a Celite pad, washing with MeOH (3×15 mL). The filtrate was concentrated under reduced pressure to afford 222 mg (81% yield) of the title compound.

¹H NMR (250 MHz, DMSO-d6) δ[ppm] 1.49-1.79 (m, 4H), 2.79-2.99 (m, 1H), 3.35-3.50 (m, 2H), 3.81-4.02 (m, 2H), 5.42 (s, 2H), 6.73-6.93 (m, 2H), 7.08-7.24 (m, 1H).

LCMS (Analytical Method A): Rt=0.92 mins; MS (ESIpos) m/z=202.90 (M+H)⁺.

### Intermediate 242A: 5-Amino-2-(4-methoxycyclohex-1-en-1-yl)benzonitrile

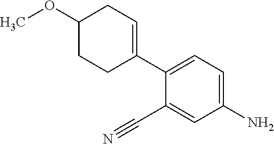 [SMILES: COC1CC=C(c2ccc(N)cc2C#N)CC1]

To a solution of 2-(4-methoxycyclohex-1-en-1-yl)-4,4,5,5-tetramethyl-1,3,2-dioxaborolane (750 mg, 3.15 mmol) and 5-amino-2-bromobenzonitrile (564 mg, 2.86 mmol) in 1,2-dimethoxyethane (13 mL) and water (2.5 mL) in a round bottomed flask equipped with condenser were added Pd(PPh₃)₂Cl₂ (40 mg, 0.057 mmol) and potassium carbonate (1.19 g, 8.59 mmol). The mixture was heated to 100° C. for 1 hour. After cooling to room temperature, the reaction was quenched by pouring onto aqueous saturated NaHCO₃. After extraction with EtOAc (×3), the combined organic extracts were washed with brine, dried (MgSO₄), filtered and concentrated. The residue was purified by Biotage Isolera™ chromatography (using a gradient of eluents; 7-80% EtOAc in heptane) to give the title product (652 mg, 82% yield) as orange oil.

¹H NMR (500 MHz, Chloroform-d) 6 [ppm] 7.09 (d, J=8.4 Hz, 1H), 6.88 (d, J=2.5 Hz, 1H), 6.80 (dd, J=8.4, 2.5 Hz, 1H), 5.83-5.76 (m, 1H), 3.79 (s, 2H), 3.62-3.52 (m, 1H), 3.41 (s, 3H), 2.61-2.53 (m, 1H), 2.52-2.36 (m, 2H), 2.24-2.15 (m, 1H), 2.10-2.03 (m, 1H), 1.83-1.73 (m, 1H).

LCMS (Analytical Method A): Rt=1.04 min; MS (ESipos) m/z=228 (M+H)⁺.

### Intermediate 243A: 5-Amino-2-(cis-4-methoxycyclohexyl)benzonitrile and Intermediate 243B: 5-Amino-2-(trans-4-methoxycyclohexyl) benzonitrile

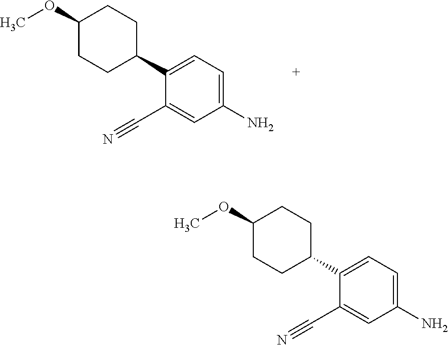 [SMILES: CO[C@H]1CC[C@@H](c2ccc(N)cc2C#N)CC1 ; CO[C@H]1CC[C@H](c2ccc(N)cc2C#N)CC1]

To a solution of racemic 5-amino-2-(4-methoxycyclohex-1-en-1-yl)benzonitrile (Intermediate 242A, 650 mg, 2.85 mmol) in ethanol (20 mL) was added 10% palladium on carbon (30 mg) and the mixture was stirred at room temperature under an atmosphere of hydrogen gas for 18 hours. More 10% palladium on carbon (30 mg) was then added and the mixture was stirred for another 24 hours under an atmosphere of hydrogen gas. More 10% palladium on carbon (30 mg) was added and the mixture was stirred for another 24 hours under an atmosphere of hydrogen gas. The mixture was diluted with ethyl acetate, filtered over Celite and further eluted with ethyl acetate. After concentration of the mother liquors, the residue was purified by Biotage Isolera™ chromatography (using a gradient of eluents; 7-60% EtOAc in heptane) to give 5-amino-2-(cis-4-methoxycyclohexyl)benzonitrile as pale yellow solid (315 mg, 48% yield) and 5-amino-2-(trans-4-methoxycyclohexyl)benzonitrile as pale brown solid (58 mg, 9% yield).

### Intermediate 243A: 5-Amino-2-(cis-4-methoxycyclohexyl)benzonitrile

¹H NMR (500 MHz, Chloroform-d) 6 [ppm] 7.19 (d, J=8.4 Hz, 1H), 6.87 (d, J=2.4 Hz, 1H), 6.84 (dd, J=8.4, 2.6 Hz, 1H), 3.80 (s, 2H), 3.56-3.49 (m, 1H), 3.34 (s, 3H), 2.93-2.84 (m, 1H), 2.10-2.02 (m, 2H), 1.80-1.68 (m, 2H), 1.65-1.52 (m, 4H).

LCMS (Analytical Method A): Rt=1.10 mins; MS (ESipos) m/z=231 (M+H)⁺.

### Intermediate 243B: 5-amino-2-(trans-4-methoxycyclohexyl)benzonitrile

¹H NMR (500 MHz, Chloroform-d) 6 [ppm] 7.10 (d, J=8.5 Hz, 1H), 6.89-6.87 (m, 1H), 6.84-6.81 (m, 1H), 3.82 (s, 2H), 3.38 (s, 3H), 3.24-3.15 (m, 1H), 2.88-2.80 (m, 1H), 2.21-2.13 (m, 2H), 1.96-1.89 (m, 2H), 1.54-1.30 (m, 4H).

LCMS (Analytical Method A): Rt=1.06 min; MS (ESipos) m/z=231 (M+H)⁺.

### Intermediate 244A: 4-(trans-4-methoxycyclohexyl)-3-(1H-tetrazol-5-yl)aniline

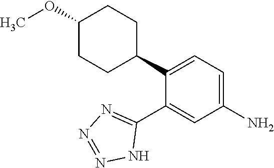 [SMILES: CO[C@H]1CC[C@H](c2ccc(N)cc2-c2nnn[nH]2)CC1]

A suspension of 5-amino-2-(trans-4-methoxycyclohexyl)benzonitrile (58 mg, 252 □mol) and dibutyl(oxo)stannane (63 mg, 252 □mol) in para-xylene (2 mL) in a pressure tube was heated to 60° C. for 15 mins. After cooling to room temperature, trimethylsilylazide (33 □L, 252 □mol) was added and the mixture was heated to 130° C. and was stirred at that temperature for 1.5 hours. More trimethylsilylazide (33 □L, 252 □mol) was added and mixture was stirred at 130° C. for another 1.5 hours. The reaction was still not complete and more trimethylsilylazide (33 □L, 252 □mol) was added and mixture was stirred at 130° C. for another 1.5 hours. Methanol was added (˜4 mL). The mixture was stirred at RT for 30 min then left standing overnight at room temperature. The volatiles were removed under reduced pressure and the residue was purified by Biotage Isolera™ chromatography (using a gradient of eluents; 1-50% methanol in dichloromethane) to give the title compound as brown solid (58 mg, 84% yield).

¹H NMR (500 MHz, Chloroform-d) 6 [ppm] 7.17 (d, J=8.4 Hz, 1H), 6.92 (s, 1H), 6.80 (d, J=7.7 Hz, 1H), 3.49 (s, 1H), 3.37 (s, 3H), 3.28-3.18 (m, 1H), 2.86-2.74 (m, 1H), 2.15-2.08 (m, 2H), 1.86-1.77 (m, 2H), 1.47-1.39 (m, 2H), 1.27-1.18 (m, 2H).

LCMS (Analytical Method A): Rt=0.86 mins; MS (ESIpos) m/z=274 (M+H)⁺.

### Intermediate 245A: 4-(cis-4-methoxycyclohexyl)-3-(1H-tetrazol-5-yl)aniline

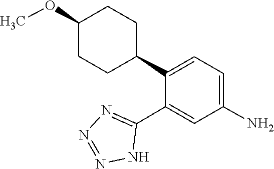 [SMILES: CO[C@H]1CC[C@@H](c2ccc(N)cc2-c2nnn[nH]2)CC1]

A suspension of 5-amino-2-(cis-4-methoxycyclohexyl)benzonitrile (315 mg, 1.37 mmol) and dibutyl(oxo)stannane (340 mg, 1.37 mmol) in para-xylene (8 mL) in a pressure tube was heated to 60° C. for 15 mins. After cooling to room temperature, trimethylsilylazide (180 □L, 1.37 mmol) was added and the mixture was heated to 130° C. and was stirred at that temperature for 1.5 hours. More trimethylsilylazide (180 □L, 1.37 mmol) was then added and mixture was stirred at 130° C. for another 1.5 hours. The reaction was still not complete and more trimethylsilylazide (180 □L, 1.37 mmol) was added and the mixture was stirred at 130° C. for another 1.5 hours. Methanol was added (˜4 mL). The mixture was stirred at room temperature for 30 mins then left standing overnight at room temperature. The volatiles were removed under reduced pressure and the residue was purified by acidic preparative HPLC (method B). The residue was then stirred in methyl tert-butyl ether and the filtrate was separated and concentrated at reduced pressure. The solid obtained was again stirred in methyl tert-butyl ether and the filtrate was separated and concentrated at reduced pressure. The solid obtained was then stirred in diethyl ether and filtered to remove the remaining solid. Concentration of the filtrate gave the title compound (127 mg, 27% yield) as a white solid.

¹H NMR (500 MHz, Methanol-d4) δ [ppm] 7.26 (d, J=8.5 Hz, 1H), 6.92 (dd, J=8.5, 2.5 Hz, 1H), 6.78 (d, J=2.5 Hz, 1H), 3.52-3.41 (m, 1H), 3.33 (s, 3H), 2.76-2.60 (m, 1H), 2.06-1.91 (m, 2H), 1.84-1.63 (m, 2H), 1.53-1.31 (m, 4H).

LCMS (Analytical Method F): Rt=1.60 mins; MS (ESipos) m/z=274 (M+H)⁺.

### Intermediate 246A: 2-(4-Bromopyridin-2-yl)propan-2-ol

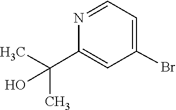

Methyl 4-bromopyridine-2-carboxylate (1.0 g, 4.6 mmol) was stirred in THF (50 mL) and the material remained insoluble. The mixture was cooled to 0° C. and methylmagnesium bromide (7.3 mL of a 1.4M solution in THF/Toluene) was added dropwise over 5 mins. The resulting mixture was allowed to warm to RT and then stirred for overnight giving a cloudy orange solution. The mixture was quenched with water and a thick brown gum formed. The solids were dissolved in EtOAc (100 mL) and water (50 mL) and the organic layer was then separated and washed with brine (2×30 mL), dried (Na₂SO₄), filtered and concentrated at reduced pressure giving the desired product (573 mg, 57% yield) as a pale yellow oil.

¹H NMR (500 MHz, Chloroform-d) 6 [ppm] 8.34 (d, J=5.3 Hz, 1H), 7.61-7.55 (m, 1H), 7.37 (dd, J=5.3, 1.8 Hz, 1H), 4.49 (s, 1H), 1.54 (s, 6H).

LCMS (Analytical Method A): Rt=0.68 mins; MS (ESipos) m/z=217 (M+H)⁺.

### Intermediate 247A: 4-Bromo-2-{2-[(trimethylsilyl)oxy]propan-2-yl}pyridine

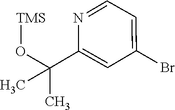

2-(4-Bromopyridin-2-yl)propan-2-ol (456 mg, 2.11 mmol), triethylamine (0.6 mL, 4.22 mmol) and 4-dimethylaminopyridine (13 mg, 0.11 mmol) were dissolved in DCM (15 mL) and cooled to 0° C. To this was added chlorotrimethylsilane (0.40 mL, 3.2 mmol) dropwise and the resulting solution was stirred at 0° C. for 30 mins. The reaction was quenched by dropwise addition of sat. aq. NaHCO₃ (2 mL) and the mixture was warmed to RT. The organics were diluted with DCM (30 mL) and washed with sat. aq. NaHCO₃ (2×20 ml), dried (Na₂SO₄), filtered and concentrated at reduced pressure. The residue was purified by flash chromatography (SiO₂, EtOAc/heptane 5-30%) to afford 373 mg (61% yield) of the title compound as a colourless oil.

¹H NMR (500 MHz, Chloroform-d) 6 [ppm] 8.32 (d, J=5.2 Hz, 1H), 7.85-7.81 (m, 1H), 7.29 (dd, J=5.2, 1.9 Hz, 1H), 1.59 (s, 6H), 0.18 (s, 9H).

LCMS (Analytical Method A): Rt=3.62 mins; MS (ESipos) m/z=289 (M+H)⁺.

### Intermediate 248A: (6-{2-Methyl-2-[(trimethylsilyl)oxy]propyl}pyridin-3-yl)boronic acid

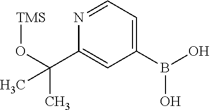 [SMILES: CC(C)(O[Si](C)(C)C)c1cc(B(O)O)ccn1]

4-Bromo-2-{2-[(trimethylsilyl)oxy]propan-2-yl}pyridine (1.0 g, 3.3 mmol) was dissolved in THF (10 mL) and cooled to −78° C. n-Butyllithium (1.46 mL of a 2.5 M solution, 3.63 mmol) was added dropwise giving a yellow solution. This was stirred at −78° C. for 15 minutes and then triisopropyl borate (0.92 mL, 1.29 mmol) was added dropwise. The resulting solution was left to stir for 15 minutes then raised to room temperature and stirred for 1 h giving an orange solution. This was quenched via addition MeOH (2 mL) and the resulting mixture was concentrated at reduced pressure. The residue was purified by flash chromatography (SiO2, MeOH/DCM 5-100%) to afford 290 mg (33% yield) of the title compound as a pale yellow solid.

¹H NMR (500 MHz, Methanol-d4) δ [ppm] 8.50 (s, 1H), 7.80 (dd, J=7.6, 1.7 Hz, 1H), 7.17 (d, J=7.6 Hz, 1H), 2.83 (s, 2H), 1.24 (s, 6H), 0.04 (s, 9H).

LCMS (Analytical Method D): Rt=0.99 mins; MS (ESipos) m/z=268 (M+H)⁺.

### Intermediate 249A: 2-(difluoromethyl)-5-(4,4,5,5-tetramethyl-1,3,2-dioxaborolan-2-yl)-pyridine

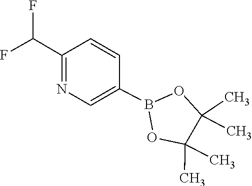 [SMILES: CC1(C)OB(c2ccc(C(F)F)nc2)OC1(C)C]

To a solution of 5-bromo-2-(difluoromethyl)pyridine (1.0 g, 4.8 mmol) and bis(pinacolato)diboron (1.34 g, 5.3 mmol) in dioxane (5 mL) was added potassium acetate (1.4 g, 14.4 mmol) at room temperature. Nitrogen gas was bubbled through the mixture for 5 mins and 1,1′-bis(diphenylphosphino)ferrocenepalladium(II) chloride (264 mg, 0.36 mmol) was then added. The mixture was heated at 100° C. for 1 hour. The reaction mixture was then diluted with EtOAc (50 mL), filtered over Celite and washed with EtOAc (50 mL). The filtrate was concentrated at reduced pressure and the residue was purified by Biotage Isolera™ chromatography [SNAP Cartridge KP-Sil 50 g; 0-100% EtOAc in heptane, 16 column volumes]. The product containing fractions were combined and concentrated in vacuo to afford the title compound (1.15 g, 89% yield) as pale yellow crystalline solid.

¹H NMR (500 MHz, Chloroform-d) 6 [ppm] 8.97 (s, 1H), 8.21 (dd, J=7.7, 1.4 Hz, 1H), 7.62 (d, J=7.7 Hz, 1H), 6.64 (t, J=55.4 Hz, 1H), 1.36 (s, 12H).

LCMS (Analytical Method A): Rt=0.78 mins, MS (ESIpos) m/z=173.9 (Mass of boronic acid+H)⁺.

### Intermediate 250A: 2-(1,1-Difluoroethyl)-5-(4,4,5,5-tetramethyl-1,3,2-dioxaborolan-2-yl)pyridine

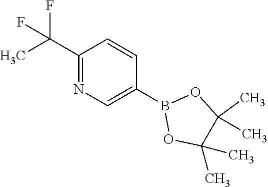 [SMILES: CC(F)(F)c1ccc(B2OC(C)(C)C(C)(C)O2)cn1]

To a solution of 5-bromo-2-(1,1-difluoroethyl)pyridine (675 mg, 2.68 mmol) in 1,4-dioxane (12 mL) was added bis(pinacolato)diboron (747 mg, 2.94 mmol) and potassium acetate (788 mg, 8 mmol). The resulting mixture was degassed for five minutes with nitrogen prior to the addition of Pd(dppf).CH₂Cl₂ (131 mg, 0.16 mmol). The mixture was heated to 100° C. and was stirred at that temperature for 2.5 hours. The reaction was allowed to cool to room temperature, at which point it was filtered through Celite, washing with MeOH (3×10 mL).

The filtrate was concentrated under reduced pressure and purified by Biotage Isolera™ chromatography (silica gel, eluting with heptanes-EtOAc, 1:0 to 0:1) to afford 416 mg (52% yield) of the title compound as an off-white powder.

¹H NMR (250 MHz, DMSO-d6) δ[ppm] 1.32 (s, 12H), 1.99 (t, J=19.1 Hz, 3H), 7.71 (dd, J=7.8, 0.9 Hz, 1H), 8.18 (dd, J=7.8, 1.7 Hz, 1H), 8.79-8.87 (m, 1H).

LCMS (Analytical Method A): Rt=0.87 mins; mass ion not observed.

### Intermediate 251A: 5-bromo-2-(1,1-difluoropropyl)pyridine

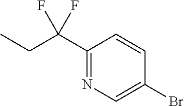

To a solution of 1-(5-bromopyridin-2-yl)propan-1-one (5.25 g, 24.5 mmol) dissolved in 1,2-dichloroethane (61.5 mL) under nitrogen was added diethylaminosulfur trifluoride (12.96 mL, 98.1 mmol) dropwise giving an orange solution. The reaction was then warmed to 60° C. and stirred at this temperature for 20 hours. The mixture was cooled to room temperature and diluted with 2M aqueous sodium hydroxide solution. The organic layer was removed and washed with saturated aqueous sodium chloride solution, dried (MgSO₄), filtered and concentrated at reduced pressure giving a thick dark brown oil. This was purified via silica flash column chromatography (using a gradient or eluents; 98:2 to 80:20 heptane/TBME) giving the title product (3.25 g, 50% yield) as a brown oil.

¹H NMR (500 MHz, DMSO-d6) δ 8.83 (d, J=2.1 Hz, 1H), 8.25 (dd, J=8.4, 2.3 Hz, 1H), 7.66 (d, J=8.4 Hz, 1H), 2.38-2.21 (m, 2H), 0.91 (t, J=7.5 Hz, 3H).

LCMS (Analytical Method A): Rt=1.21 mins, MS (ESIpos): m/z=238 (M+H)⁺.

### Intermediate 252A: 2-(1,1-difluoropropyl)-5-(4,4,5,5-tetramethyl-1,3,2-dioxaborolan-2-yl)pyridine

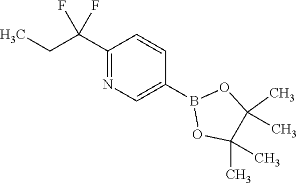 [SMILES: CCC(F)(F)c1ccc(B2OC(C)(C)C(C)(C)O2)cn1]

To a solution of 5-bromo-2-(1,1-difluoropropyl)pyridine (2.1 g, 8.9 mmol) and bis(pinacolato)diboron (2.5 g, 9.8 mmol) in dioxane (50 mL) was added and potassium acetate (2.6 g, 26.7 mmol) at room temperature. Nitrogen gas was bubbled through the mixture for 5 mins and 1,1′-bis(diphenylphosphino)ferrocenepalladium(II) chloride (78 mg, 0.11 mmol) was then added. The mixture was heated at 100° C. for 1 hour. LCMS indicated only starting material remaining. The mixture was cooled to room temperature and degassed again by bubbling nitrogen gas through the mixture for 5 mins. Further 1,1′-bis(diphenylphosphino)ferrocenepalladium(II) chloride (200 mg, 0.27 mmol) was added and the mixture was heated for 45 minutes at 100° C. LCMS showed a 2:1 mixture of product/starting material. 1,1′-bis(diphenylphosphino)ferrocenepalladium(II) chloride (90 mg, 1.23 mmol) was added and the mixture was heated for 30 minutes at 100° C. and −85% conversion was observed. The reaction mixture was cooled to room temperature and diluted with EtOAc (50 mL), filtered over Celite and washed with EtOAc (50 mL). The filtrate was concentrated at reduced pressure and the residue was purified by Biotage Isolera™ chromatography [SNAP Cartridge KP-Sil 50 g; 0-30% TBME in heptane]. The product containing fractions were combined and concentrated in vacuo to afford the desired compound (1.48 g, 41% yield) as a colourless oil. The product crystallised on standing giving a white solid. NMR showed the product contained 1 equivalent of pinacol giving a 70% purity by NMR.

¹H NMR (500 MHz, DMSO-d6) δ[ppm] 8.84 (s, 1H), 8.18 (dd, J=7.8, 1.7 Hz, 1H), 7.77-7.56 (m, 1H), 2.41-2.16 (m, 2H), 1.32 (s, 12H), 0.89 (t, J=7.5 Hz, 3H).

LCMS (Analytical Method A): Rt=0.91 mins, MS (ESIpos): m/z=202 (Mass boronic acid+H)⁺.

### Intermediate 253A: 5,5,5-trifluoro-1-(trimethylsilyl)pent-1-yn-3-one

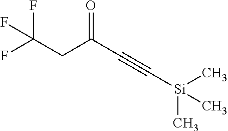

To a stirred solution of ethyne-1,2-diylbis(trimethylsilane) (1.0 g, 5.868 mmol) and 3,3,3-trifluoropropanoyl chloride (0.946 g, 6.455 mmol) in dichloromethane (15 mL) was added aluminium trichloride (0.939 g, 7.042 mmol) at 0° C. The reaction mixture was stirred at 0° C. for 1 hour and then poured into a mixture of 2M HCl (20 mL) and crushed ice (˜20 g). Dichloromethane (20 ml) was added and the mixture was left standing for approximately 2 hours (by then 2 relatively clear layers had formed). The organic layer was separated and the aqueous layer was extracted with dichloromethane (20 mL). The combined organic layers were washed with brine (20 mL), dried (MgSO₄) and concentrated at reduced pressure to afford the title compound (1.18 g, 75% yield) as pale yellow oil.

¹H NMR (500 MHz, Chloroform-d) 6 [ppm] 3.38 (q, J=10.0 Hz, 2H), 0.26 (s, 9H).

### Intermediate 254A: 3-(2,2,2-trifluoroethyl)-1H-pyrazole

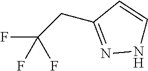

To a solution 5,5,5-trifluoro-1-(trimethylsilyl)pent-1-yn-3-one (1.18 g, 4.82 mmol) in ethanol (10 mL) was added hydrazine hydrate (0.469 mL, 9.63 mmol) at room temperature (an exothermic reaction was observed). The mixture was stirred for 1 hour and then concentrated at reduced pressure (˜50 mbar). The residue was dissolved in dichloromethane (50 mL), washed with 2M K₂CO₃ (20 mL) and brine (20 mL), dried (Na₂SO₄) and concentrated at reduced pressure (50 mbar) to afford the title compound (630 mg, 73% yield) as yellow oil.

¹H NMR (500 MHz, Chloroform-d) 6 [ppm] 7.57 (d, J=2.2 Hz, 1H), 6.35 (d, J=1.8 Hz, 1H), 3.51 (q, J=10.7 Hz, 2H).

LCMS (Analytical Method A): Rt=0.81 mins; MS (ESIpos) m/z=150.9 (M+H)⁺.

### Intermediate 255A: tert-Butyl 1-(1-hydroxy-4,4-dimethylcyclohexyl)cyclopropane carboxylate

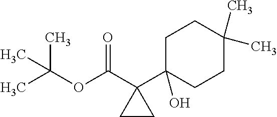 [SMILES: CC1(C)CCC(O)(C2(C(=O)OC(C)(C)C)CC2)CC1]

To a solution of tert-butyl cyclopropanecarboxylate (3.16 g, 22.00 mmol) in THF (50 mL) was added a 2.5M solution of n-butyllithium in hexanes (11.0 mL, 22 mmol) at −78° C. The resulting yellow solution was stirred for 1 hour at this temperature before a solution of 4,4-dimethylcyclohexan-1-one (2.776 g, 22 mmol) in THF (5 mL) was added drop-wise over a period of 10 minutes. The mixture was stirred for another 2 hours at −78° C. and then allowed to warm slowly to RT. The mixture was stirred overnight at RT and monitored by TLC (EtOAc/heptane 1:9). The reaction was quenched by addition of saturated ammonium chloride solution and diluted with TBME (150 mL). The organic layer was separated, washed with brine (2×30 mL), dried (MgSO₄), filtered and concentrated under reduced pressure.

The residue was purified by flash chromatography (SiO₂, EtOAc/heptane 0-20%). The bulk of the starting material was separated, but the desired product and two other (higher running) spots were collected in the same fractions. The product containing fractions were combined and re-purified by flash chromatography (SiO₂, heptane/DCM 0-100%) to afford 2.10 g (34% yield) of the title compound as a colourless oil.

¹H NMR (500 MHz, Chloroform-d) 6 [ppm] 3.88 (s, 1H), 1.61-1.53 (m, 2H), 1.52-1.44 (m, 2H), 1.39-1.30 (m, 11H), 1.10-1.03 (m, 2H), 1.02-0.97 (m, 2H), 0.88-0.85 (m, 2H), 0.85 (s, 3H), 0.77 (s, 3H).

### Intermediate 256A: tert-Butyl 1-(4,4-dimethylcyclohex-1-en-1-yl)cyclopropanecarboxylate

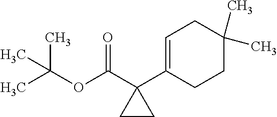 [SMILES: CC1(C)CC=C(C2(C(=O)OC(C)(C)C)CC2)CC1]

To a solution of tert-butyl 1-(1-hydroxy-4,4-dimethylcyclohexyl)cyclopropanecarboxylate (2.0 g, 7.38 mmol) in toluene (15 mL) was added methyl N-(triethylammoniumsulfonyl) carbamate (Burgess reagent) (2.11 g, 8.85 mmol) at room temperature The resulting mixture was heated at reflux for 1 hour (TLC (EtOAc/heptane 1:9) indicated starting material consumed, one major new spot). After cooling to RT, the reaction was diluted with EtOAc (60 ml) and water (40 ml). The organic layer was separated, washed with brine (30 mL), dried (MgSO₄) and concentrated under reduced pressure. The residue was purified by flash chromatography (SiO₂, DCM/heptane 0-100%) to afford 1.65 g (85% yield) of the title compound as a colourless oil.

¹H NMR (500 MHz, Chloroform-d) 6 [ppm] 5.55-5.44 (m, 1H), 2.18-2.07 (m, 2H), 1.83-1.72 (m, 2H), 1.42 (s, 9H), 1.38-1.33 (m, 2H), 1.19 (q, J=3.6 Hz, 2H), 0.91 (s, 6H), 0.83-0.79 (m, 2H).

### Intermediate 257A: tert-Butyl 1-(4,4-dimethylcyclohexyl)cyclopropanecarboxylate and tert-butyl 2-(4,4-dimethylcyclohexyl)butanoate

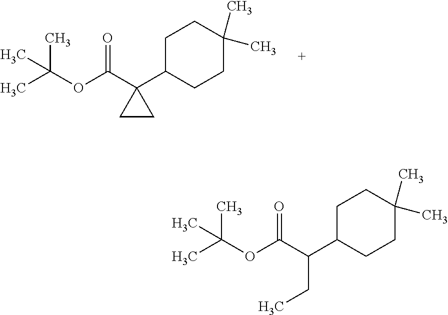 [SMILES: CC1(C)CCC(C2(C(=O)OC(C)(C)C)CC2)CC1 ; CCC(C(=O)OC(C)(C)C)C1CCC(C)(C)CC1]

To a degassed solution of tert-butyl 1-(4,4-dimethylcyclohex-1-en-1-yl)cyclopropane carboxylate (1.50 g, 5.99 mmol) in EtOH (30 mL) was added Pd/C (5%, 200 mg, 0.047 mmol).

The mixture was stirred at room temperature under an atmosphere of hydrogen for 24 hours.

The catalyst was removed by filtration (Celite) and washed with EtOH (50 mL). The filtrate was concentrated in vacuo and the residue was purified by silica flash column chromatography (SiO₂, DCM/heptane 0-100%) giving 1.46 g (48% yield) of a 1:1 mixture of tert-butyl 1-(4,4-dimethylcyclohexyl)cyclopropanecarboxylate and tert-butyl 2-(4,4-dimethylcyclohexyl)butanoate as colourless oil. The mixture was used in the next step without further purification.

### Intermediate 258A: 1-(4,4-Dimethylcyclohexyl)cyclopropanecarboxylic acid and 2-(4,4-dimethylcyclohexyl)butanoic acid

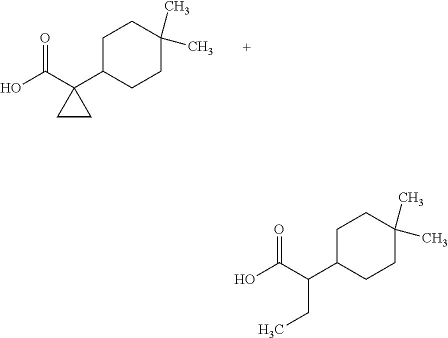

To a solution of a 1:1 mixture of tert-butyl 1-(4,4-dimethylcyclohexyl)cyclopropane carboxylate and tert-butyl 2-(4,4-dimethylcyclohexyl)butanoate (1.46 g, 2.9 mmol) in dichloromethane (3 mL) was added trifluoroacetic acid (3 mL, 38.9 mmol). The mixture was stirred at room temperature for 2 hours. The volatiles were removed under reduced pressure to afford 1.12 g (99% yield) of a 1:1 mixture of 1-(4,4-Dimethylcyclohexyl)cyclopropanecarboxylic acid and 2-(4,4-dimethylcyclohexyl)butanoic acid as colourless oil. The mixture was used in the next step without further purification.

### Intermediate 259A: 1-[2-fluoro-4-(trifluoromethyl)phenyl]cyclopropanecarbonitrile

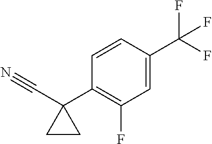 [SMILES: N#CC1(c2ccc(C(F)(F)F)cc2F)CC1]

Potassium hydroxide (22.1 g, 0.39 mol) was dissolved in water (32 mL) and added slowly to a mixture of tetra-n-butylammonium bromide (317 mg, 0.98 mmol), [2-fluoro-4-(trifluoromethyl)phenyl]acetonitrile (10.0 g, 49.2 mmol) and 1-bromo-2-chloroethane (21.2 g, 148 mmol) over 30 mins. An exotherm was observed on addition and the internal temperature was maintained below 80° C. using an ice bath. A dark red solution and white precipitate formed on complete addition and the mixture was allowed to cool to room temperature. The reaction was stirred for 2 hours then the mixture was diluted with water (100 mL) and EtOAc (300 mL) and the organic layer was decanted off then washed with brine (2×75 mL) then dried (Na₂SO₄), filtered and concentrated at reduced pressure. The residue was purified by Biotage Isolera™ chromatography (using a gradient of eluents; 98:2 to 85:15 heptane/EtOAc) giving the title compound (11.1 g, quantitative yield) as a yellow oil.

¹H NMR (500 MHz, DMSO-d6) δ[ppm] 7.85-7.76 (m, 1H), 7.76-7.66 (m, 1H), 7.66-7.55 (m, 1H), 1.80-1.71 (m, 2H), 1.58-1.51 (m, 2H).

LCMS (Analytical method A): Rt=0.62 mins, MS (ESIpos) m/z=210 (M+H)⁺.

### Intermediate 260A: 1-[2-fluoro-4-(trifluoromethyl)phenyl]cyclopropanecarboxylic acid

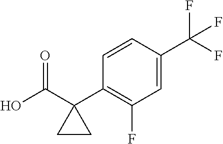 [SMILES: O=C(O)C1(c2ccc(C(F)(F)F)cc2F)CC1]

1-[2-Fluoro-4-(trifluoromethyl)phenyl]cyclopropanecarbonitrile 259A (11.1 g, 48 mmol) was dissolved in concentrated HCl (50 mL) and heated at 90° C. overnight. The mixture was diluted with water (150 mL) and extracted with EtOAc (150 mL). The organics were then separated and washed with brine (2×50 mL), dried (Na₂SO₄), filtered and concentrated at reduced pressure. The residue was crystallised in heptane giving the desired product (8.4 g, 70% yield) as a white solid.

¹H NMR (250 MHz, DMSO-d6) δ[ppm] 12.61 (s, 1H), 7.72-7.40 (m, 3H), 1.64-1.46 (m, 2H), 1.31-1.15 (m, 2H).

LCMS (Analytical method A); RT=1.13 mins, MS (ESIpos) m/z=289 (M+MeCN)⁺.

### Intermediate 261A: 1-[2-Fluoro-4-(trifluoromethyl)phenyl]cyclopropanecarbonyl chloride

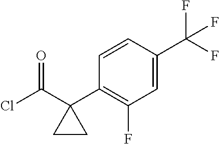 [SMILES: O=C(Cl)C1(c2ccc(C(F)(F)F)cc2F)CC1]

1-[2-Fluoro-4-(trifluoromethyl)phenyl]cyclopropanecarboxylic acid 260A (8.4 g, 33.8 mmol) was stirred in thionyl chloride (20 mL) and DMF (4 drops) was added. The mixture was stirred at room temperature for 1 hour then heated to 90° C. for 1 hour. NMR (sample prepared from MeOH) shows complete conversion to the methyl ester. The mixture was then concentrated at reduced pressure. The residue was diluted with heptane (5 mL) and concentrated twice to removed excess thionyl chloride giving the desired product (8.40 g, 93% yield) as an orange oil.

This was used in the next step without further purification.

¹H NMR (250 MHz, Chloroform-d) 6 [ppm] 7.51-7.30 (m, 3H), 2.11-1.99 (m, 2H), 1.53-1.43 (m, 2H).

### Example 1: 1-(3-Chlorophenyl)-N-[3′,4′-dimethoxy-2-(1H-tetrazol-5-yl)biphenyl-4-yl]cyclopropanecarboxamide

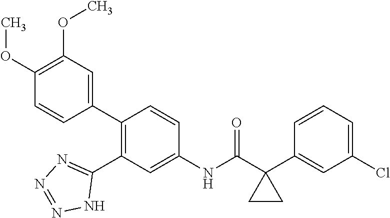 [SMILES: COc1ccc(-c2ccc(NC(=O)C3(c4cccc(Cl)c4)CC3)cc2-c2nnn[nH]2)cc1OC]

3′,4′-Dimethoxy-2-(1H-tetrazol-5-yl)biphenyl-4-amine (Intermediate 54A, 250 mg, 0.81 mmol) and 1-(3-chlorophenyl)cyclopropane-1-carboxylic acid (198 mg, 1.0 mmol) were dissolved in DMF (4 mL) and N,N-diisopropylethylamine (0.29 □L, 1.7 mmol) and HATU (383 mg, 1.0 mmol) was added giving a yellow solution. This was stirred for overnight at RT. The mixture was diluted with EtOAc (50 mL) and washed with 2M aq. HCl (2×2 mL) and brine (2 mL), dried (Na₂SO₄), filtered and concentrated. The residue was purified via silica FCC (using a gradient of eluents; 99:1 to 90:10 DCM/MeOH) then via acidic preparative LC giving the title compound (191 mg, 48% yield) as a white solid.

¹H NMR (500 MHz, DMSO-d6) δ[ppm] 1.12-1.24 (m, 2H), 1.45-1.55 (m, 2H), 3.58 (s, 3H), 3.73 (s, 3H), 6.52-6.61 (m, 2H), 6.87 (d, J=8.2 Hz, 1H), 7.32-7.42 (m, 3H), 7.44 (d, J=1.7 Hz, 1H), 7.50 (d, J=8.4 Hz, 1H), 7.82-7.89 (m, 2H), 9.47 (s, 1H).

LCMS (Analytical Method F): Rt=3.45 mins; MS (ESIpos) m/z=476 (M+H)⁺.

In analogy to Example 1, the following examples were prepared using the corresponding amine and carboxylic acid as starting materials:

| Ex. | Structure | Name | Analytical Data |
|---|---|---|---|
| 2 | 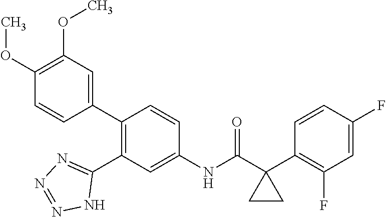 [SMILES: COc1ccc(-c2ccc(NC(=O)C3(c4ccc(F)cc4F)CC3)cc2-c2nnn[nH]2)cc1OC] | 1-(2,4- difluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.11-1.16 (m, 2H), 1.57- 1.60 (m, 2H), 3.54 (s, 3H), 3.72 (s, 3H), 6.57 (d, 1H), 6.62 (dd, 1H), 6.84 (d, 1H), 7.10 (ddt, 1H), 7.26 (ddt, 1H), 7.41 (d, 1H), 7.50- 7.56 (m, 1H), 7.68 (d, 1H), 7.79 (dd, 1H), 9.02 (s, 1H). LCMS (method 4): Rₜ = 0.75 min; MS (ESIpos) m/z = 478 (M + H)⁺. |
| 3 | 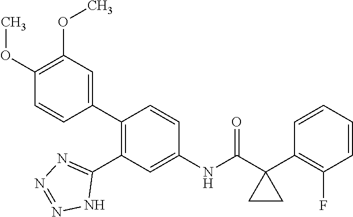 [SMILES: COc1ccc(-c2ccc(NC(=O)C3(c4ccccc4F)CC3)cc2-c2nnn[nH]2)cc1OC] | N-[3′,4′- dimethoxy-2- (1H-tetrazol-5- yl)biphenyl-4- yl]-1-(2- fluorophenyl) cyclopropane- carboxamide | ¹H NMR (400 MHz, DMSO-d6) δ [ppm] 1.15-1.18 (m, 2H), 1.58- 1.61 (m, 2H), 3.57 (s, 3H), 3.73 (s, 3H), 6.55-6.60 (m, 2H), 6.86 (d, 1H), 7.19-7.25 (m, 2H), 7.37- 7.43 (m, 1H), 7.46-7.50 (m, 1H), 7.78 (d, 1H), 7.85 (dd, 1H), 9.09 (s, 1H). LCMS (method 1): Rₜ = 1.09 min; MS (ESIpos) m/z = 460 (M + H)⁺ |
| 4 | 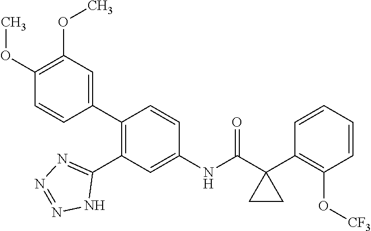 [SMILES: COc1ccc(-c2ccc(NC(=O)C3(c4ccccc4OC(F)(F)F)CC3)cc2-c2nnn[nH]2)cc1OC] | N-[3′,4′- dimethoxy-2- (1H-tetrazol-5- yl)biphenyl-4- yl]-1-[2- (trifluoro- methoxy) phenyl] cyclopropane- carboxamide | ¹H NMR (400 MHz, DMSO-d6) δ [ppm] 1.16-1.18 (m, 2H), 1.49- 1.51 (m, 2H), 3.57 (s, 3H), 3.73 (s, 3H), 6.56-6.60 (m, 2H), 6.87 (d, 1H), 7.36-7.43 (m, 2H, 7.47- 7.51 (m, 2H), 7.58 (dd, 1H), 7.75 (d, 1H), 7.83 (dd, 1H), 9.06 (s, 1H). LCMS (method 1): Rₜ = 1.20 min; MS (ESIpos) m/z = 526 (M + H)⁺ |
| 5 | 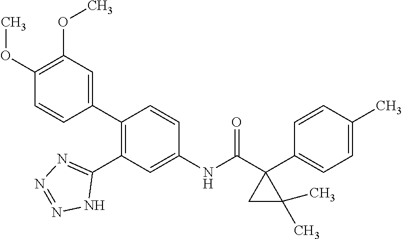 [SMILES: COc1ccc(-c2ccc(NC(=O)C3(c4ccc(C)cc4)CC3(C)C)cc2-c2nnn[nH]2)cc1OC] | N-[3′,4′- dimethoxy-2- (1H-tetrazol-5- yl)biphenyl-4- yl]-2,2- dimethyl-1-(4- methylpheny) cyclopropane carboxamide | ¹H NMR (400 MHz, DMSO-d6) δ [ppm] 0.85 (s, 3H), 1.09 (d, 1H), 1.23 (s, 3H), 1.39 (d, 1H), 2.28 (s, 3H), 3.57 (s, 3H), 3.73 (s, 3H), 6.54-6.57 (m, 2H), 6.86 (d, 1H), 7.08-7.21 (m, 2H), 7.47 (d, 2H), 7.50 (d, 1H), 7.86-7.90 (m, 2H), 9.86 (s, 1H). LCMS (method 3): Rₜ = 1.31 min; MS (ESIpos) m/z = 484 (M + H)⁺ |
| 6 | 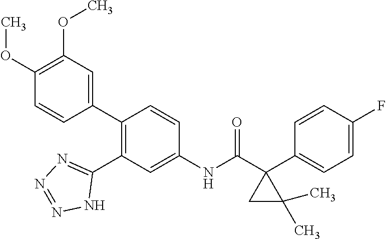 [SMILES: COc1ccc(-c2ccc(NC(=O)C3(c4ccc(F)cc4)CC3(C)C)cc2-c2nnn[nH]2)cc1OC] | N-[3′,4′- dimethoxy-2- (1H-tetrazol-5- yl(biphenyl-4- yl]-1-(4- fluorophenyl)- 2,2- dimethylcyclo- propane- carboxamide | ¹H NMR (400 MHz, DMSO-d6) δ [ppm] 0.85 (s, 3H), 1.13 (d, 1H), 1.23 (s, 3H), 1.44 (d, 1H), 3.57 (s, 3H), 3.73 (s, 3H), 6.54-6.57 (m, 2H), 6.86 (d, 1H), 7.15-7.20 (m, 2H), 7.59-7.63 (m, 2H), 7.86-7.90 (m, 2H), 9.93 (s, 1H). LCMS (method 3): Rₜ = 1.25 min; MS (ESIpos) m/z = 488 (M + H)⁺ |
| 7 | 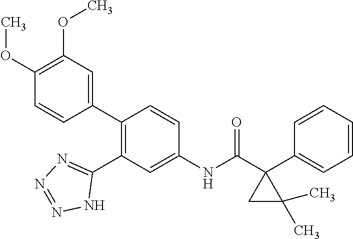 [SMILES: COc1ccc(-c2ccc(NC(=O)C3(c4ccccc4)CC3(C)C)cc2-c2nnn[nH]2)cc1OC] | N-[3′,4′- dimethoxy-2- (1H-tetrazol-5- yl)biphenyl-4- yl]-2,2- dimethyl-1- phenylcyclo- propane- carboxamide | ¹H NMR (400 MHz, DMSO-d6) δ [ppm] 0.85 (s, 3H), 1.14 (d, 1H), 1.24 (s, 3H), 1.42 (d, 1H), 3.56 (s, 3H), 3.72 (s, 3H), 6.54-6.57 (m, 2H), 6.86 (d, 1H), 7.25-7.29 (m, 1H), 7.33-7.36 (m, 2H), 7.49 (d, 1H), 7.57-7.60 (m, 2H), 7.86-7.91 (m, 2H), 9.91 (s, 1H). LCMS (method 1): Rₜ = 1.21 min; MS (ESIpos) m/z = 470 (M + H)⁺ |
| 8 | 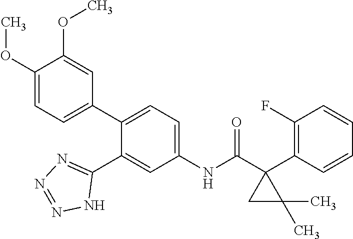 [SMILES: COc1ccc(-c2ccc(NC(=O)C3(c4ccccc4F)CC3(C)C)cc2-c2nnn[nH]2)cc1OC] | N-[3′,4′- dimethoxy-2- (1H-tetrazol-5- yl)biphenyl-4- yl]-1-(2- fluorophenyl)- 2,2- dimethylcyclo- propane- carboxamide | ¹H NMR (400 MHz, DMSO-d6) δ [ppm] 0.98 (s, 3H), 1.02 (d, 1H), 1.27 (s, 3H), 1.68 (d, 1H), 3.56 (s, 3H), 3.72 (s, 3H), 6.54-6.58 (m, 2H), 6.86 (d, 1H), 7.17-7.23 (m, 2H), 7.31-7.37 (m, 1H), 7.48 (d, 1H), 7.68 (dt, 1H), 7.84- 7.87 (m, 2H), 9.57(s, 1H). LCMS (method 4): Rₜ = 0.81 min; MS (ESIpos) m/z = 488 (M + H)⁺ |
| 9 | 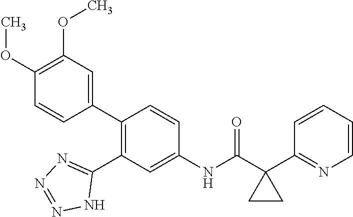 [SMILES: COc1ccc(-c2ccc(NC(=O)C3(c4ccccn4)CC3)cc2-c2nnn[nH]2)cc1OC] | N-[3′,4′- dimethoxy-2- (1H-tetrazol-5- yl)biphenyl-4- yl]-1-(pyridin-2- yl(cyclopropane carboxamide | ¹H NMR (400 MHz, DMSO-d6) δ [ppm] 1.39-1.42 (m, 2H), 1.52- 1.55 (m, 2H), 3.57 (s, 3H), 3.73 (s, 3H), 6.58 (d, 1H), 6.62 (dd, 1H), 6.87 (d, 1H), 7.28 (ddd, 1H), 7.50 (d, 1H), 7.77 (dt, 1H), 7.88 (d, 1H), 7.90 (s, 1H), 8.56 (ddd, 1H), 10.69 (s, 1H). LCMS (method 1): Rₜ = 0.85 min; MS (ESIpos) m/z = 443 (M + H)⁺ |
| 10 | 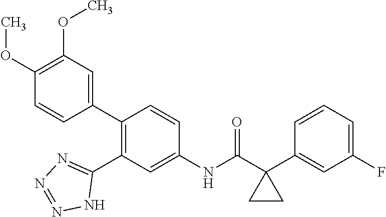 [SMILES: COc1ccc(-c2ccc(NC(=O)C3(c4cccc(F)c4)CC3)cc2-c2nnn[nH]2)cc1OC] | N-[3′,4′- dimethoxy-2- (1H-tetrazol-5- yl)biphenyl-4- yl]-1-(3- fluorophenyl) cyclopropane- carboxamide | ¹H NMR (400 MHz, DMSO-d6) δ [ppm] 1.18-1.21 (m, 2H), 1.48- 1.51 (m, 2H), 3.58 (s, 3H), 3.73 (s, 3H), 6.56-6.59 (m, 2H), 6.89 (d, 1H), 7.10-7.16 (m, 1H), 7.20- 7.27 (m, 2H), 7.38-7.44 (m, 1H), 7.51 (d, 1H), 7.85-7.88 (m, 2H), 9.45 (s, 1H). LCMS (method 3): Rₜ = 1.16 min; MS (ESIpos) m/z = 460 (M + H)⁺ |
| 11 | 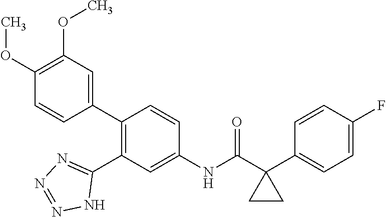 [SMILES: COc1ccc(-c2ccc(NC(=O)C3(c4ccc(F)cc4)CC3)cc2-c2nnn[nH]2)cc1OC] | N-[3′,4′- dimethoxy-2- (1H-tetrazol-5- yl)biphenyl-4- yl]-1-(4- fluorophenyl) cyclopropane- carboxamide | ¹H NMR (400 MHz, DMSO-d6) δ [ppm] 1.11-1.15 (m, 2H), 1.48- 1.50 (m, 2H), 3.57 (s, 3H), 3.73 (s, 3H), 6.54-6.60 (m, 2H), 6.87 (d, 1H), 7.17-7.22 (m, 2H), 7.44- 7.51 (m, 3H), 7.82 (d, 1H), 7.86 (dd, 1H), 9.26 (s, 1H). LCMS (method 3): Rₜ = 1.17 min; MS (ESIpos) m/z = 460 (M + H)⁺ |
| 12 | 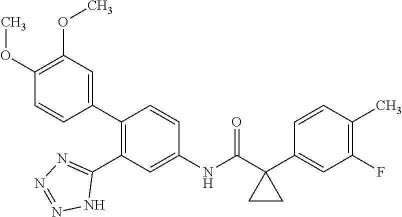 [SMILES: COc1ccc(-c2ccc(NC(=O)C3(c4ccc(C)c(F)c4)CC3)cc2-c2nnn[nH]2)cc1OC] | N-[3′,4′- dimethoxy-2- (1H-tetrazol-5- yl)biphenyl-4- yl]-1-(3-fluoro- 4- methylpheny) cyclopropane- carboxamide | ¹H NMR (400 MHz, DMSO-d6) δ [ppm] 1.14-1.16 (m, 2H), 1.46- 1.48 (m, 2H), 2.24 (d, 3H), 3.58 (s, 3H), 3.73 (s, 3H), 6.55-6.60 (m, 2H), 6.87 (d, 1H), 7.14-7.18 (m, 2H), 7.27 (t, 1H), 7.50 (d, 1H), 7.84 (d, 1H), 7.87 (dd, 1H), 9.33 (s, 1H). LCMS (method 3): Rₜ = 1.24 min; MS (ESIpos) m/z = 474 (M + H)⁺ |
| 13 | 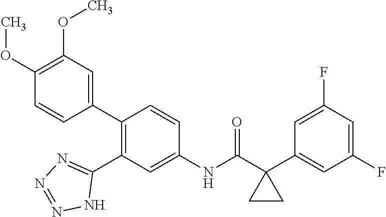 [SMILES: COc1ccc(-c2ccc(NC(=O)C3(c4cc(F)cc(F)c4)CC3)cc2-c2nnn[nH]2)cc1OC] | 1-(3,5- difluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.22-1.25 (m, 2H), 1.50- 1.53 (m, 2H), 3.57 (s, 3H), 3.73 (s, 3H), 6.56-6.60 (m, 2H), 6.87 (d, 1H), 7.11-7.21 (m, 3H), 7.50 (d, 1H), 7.82-7.87 (m, 2H), 9.43 (s, 1H). LCMS (method 3): Rₜ = 1.19 min; MS (ESIpos) m/z = 478 (M + H)⁺ |
| 14 | 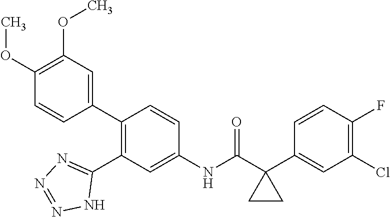 [SMILES: COc1ccc(-c2ccc(NC(=O)C3(c4ccc(F)c(Cl)c4)CC3)cc2-c2nnn[nH]2)cc1OC] | 1-(3-chloro-4- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.17-1.19 (m, 2H), 1.49- 1.51 (m, 2H), 3.56 (s, 3H), 3.73 (s, 3H), 6.56-6.61 (m, 2H), 6.86 (d, 1H), 7.41 (d, 1H), 7.42-7.48 (m, 2H), 7.63 (dd, 1H), 7.76- 7.85 (m, 2H), 9.25 (s, 1H). LCMS (method 3): Rₜ = 1.24 min; MS (ESIpos) m/z = 494 (M + H)⁺ |
| 15 | 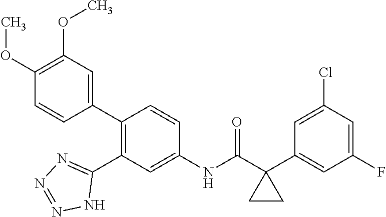 [SMILES: COc1ccc(-c2ccc(NC(=O)C3(c4cc(F)cc(Cl)c4)CC3)cc2-c2nnn[nH]2)cc1OC] | 1-(3-chloro-5- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.22-1.25 (m, 2H), 1.50- 1.52 (m, 2H), 3.57 (s, 3H), 3.73 (s, 3H), 6.56-6.61 (m, 2H), 6.86 (d, 1H), 7.24-7.28 (m, 1H), 7.33 (s, 1H), 7.37 (td, 1H), 7.49 (d, 1H), 7.80-7.86 (m, 2H), 9.43 (s, 1H). LCMS (method 3): Rₜ = 1.25 min; MS (ESIpos) m/z = 494 (M + H) |
| 16 | 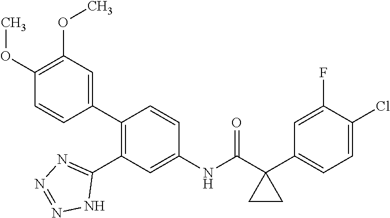 [SMILES: COc1ccc(-c2ccc(NC(=O)C3(c4ccc(Cl)c(F)c4)CC3)cc2-c2nnn[nH]2)cc1OC] | 1-(4-chloro-3- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.19-1.22 (m, 2H), 1.50- 1.53 (m, 2H), 3.57 (s, 3H), 3.73 (s, 3H), 6.56-6.60 (m, 2H), 6.87 (d, 1H), 7.29 (dd, 1H), 7.46 (dd, 1H), 7.50 (d, 1H), 7.57 (t, 1H), 7.81 (d, 1H), 7.86 (dd, 1H), 9.34 (s, 1H). LCMS (method 3): Rₜ = 1.24 min; MS (ESIpos) m/z = 494 (M + H)⁺ |
| 17 | 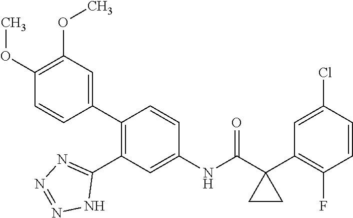 [SMILES: COc1ccc(-c2ccc(NC(=O)C3(c4cc(Cl)ccc4F)CC3)cc2-c2nnn[nH]2)cc1OC] | 1-(5-chloro-2- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.21-1.24 (m, 2H), 1.57- 1.60 (m, 2H), 3.58 (s, 3H), 3.73 (s, 3H), 6.56-6.59 (m, 2H), 6.87 (d, 1H), 7.27 (t, 1H), 7.44-7.48 (m, 1H), 7.50 (d, 1H), 7.55 (dd, 1H), 7.79 (d, 1H), 7.86 (dd, 1H), 9.22 (s, 1H). LCMS (method 3): Rₜ = 1.22 min; MS (ESIpos) m/z = 494 (M + H)⁺ |
| 18 | 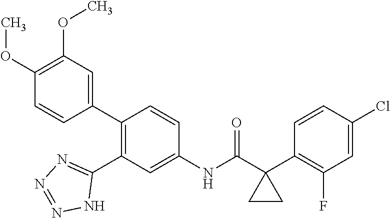 [SMILES: COc1ccc(-c2ccc(NC(=O)C3(c4ccc(Cl)cc4F)CC3)cc2-c2nnn[nH]2)cc1OC] | 1-(4-chloro-2- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.16-1.19 (m, 2H), 1.58- 1.61 (m, 2H), 3.57 (s, 3H), 3.73 (s, 3H), 6.56-6.59 (m, 2H), 6.87 (d, 1H), 7.31 (dd, 1H), 7.45 (dd, 1H), 7.48-7.53 (m, 3H), 7.78 (d, 1H), 7.86 (dd, 1H), 9.13 (s, 1H). LCMS (method 3): Rₜ = 1.23 min; MS (ESIpos) m/z = 494 (M + H)⁺ |
| 19 | 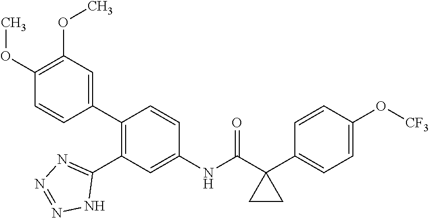 [SMILES: COc1ccc(-c2ccc(NC(=O)C3(c4ccc(OC(F)(F)F)cc4)CC3)cc2-c2nnn[nH]2)cc1OC] | N-[3′,4′- dimethoxy-2- (1H-tetrazol-5- yl)biphenyl-4- yl]-1-[4- (trifluoromethoxy) phenyl] cyclopropane- carboxamide | ¹H NMR (400 MHz, DMSO-d6) δ [ppm] 1.16-1.19 (m, 2H), 1.50- 1.53 (m, 2H), 3.56 (s, 3H), 3.73 (s, 3H), 6.56-6.60 (m, 2H), 6.86 (d, 1H), 7.35 (d, 2H), 7.48 (d, 1H), 7.53 (d, 2H), 7.80-7.85 (m, 2H), 9.45 (s, 1H). LCMS (method 3): Rₜ = 1.29 min; MS (ESIpos) m/z = 526 (M + H)⁺ |
| 20 | 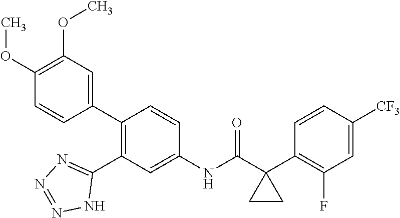 [SMILES: COc1ccc(-c2ccc(NC(=O)C3(c4ccc(C(F)(F)F)cc4F)CC3)cc2-c2nnn[nH]2)cc1OC] | N-[3′,4′- dimethoxy-2- (1H-tetrazol-5- yl)biphenyl-4- yl]-1-[2-fluoro- 4- (trifluoromethyl) phenyl] cyclopropane- carboxamide | ¹H NMR (400 MHz, DMSO-d6) δ [ppm] 1.23-1.26 (m, 2H), 1.63- 1.66 (m, 2H), 3.57 (s, 3H), 3.73 (s, 3H), 6.56-6.59 (m, 2H), 6.87 (d, 1H), 7.49 (d, 1H), 7.59-7.63 (m, 1H), 7.68-7.73 (m, 2H), 7.77 (d, 1H), 7.84 (dd, 1H), 9.21 (s, 1H). LCMS (method 3): Rₜ = 1.27 min; MS (ESIpos) m/z = 528 (M + H)⁺ |
| 21 | 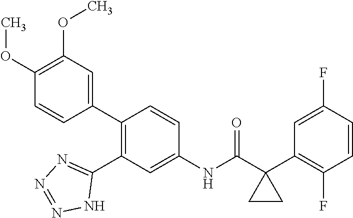 [SMILES: COc1ccc(-c2ccc(NC(=O)C3(c4cc(F)ccc4F)CC3)cc2-c2nnn[nH]2)cc1OC] | 1-(2,5- difluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.20-1.23 (m, 2H), 1.57- 1.60 (m, 2H), 3.58 (s, 3H), 3.73 (s, 3H), 6.56-6.58 (m, 2H), 6.87 (d, 1H), 7.21-7.30 (m, 2H), 7.33- 7.38 (m, 1H), 7.51 (d, 1H), 7.79 (d, 1H), 7.87 (dd, 1H), 9.19 (s, 1H). LCMS (method 3): Rₜ = 1.15 min; MS (ESIpos) m/z = 478 (M + H)⁺ |
| 22 | 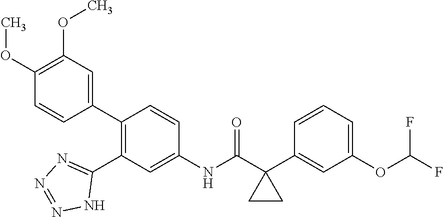 [SMILES: COc1ccc(-c2ccc(NC(=O)C3(c4cccc(OC(F)F)c4)CC3)cc2-c2nnn[nH]2)cc1OC] | 1-[3- (difluoromethoxy) phenyl]-N- [3′,4′- dimethoxy-2- (1H-tetrazol-5- yl)biphenyl-4- yl]cyclopropane carboxamide | ¹H NMR (400 MHz, DMSO-d6) δ [ppm] 1.17-1.20 (m, 2H), 1.45- 1.51 (m, 2H), 3.57 (s, 3H), 3.73 (s, 3H), 6.56-6.59 (m, 2H), 6.87 (d, 1H), 7.07-7.12 (m, 2H), 7.19- 7.31 (m, 2H), 7.40-7.45 (m, 2H), 7.51 (d, 1H), 7.84-8.87 (m, 1H), 9.47 (s, 1H). LCMS (method 3): Rₜ = 1.19 min; MS (ESIpos) m/z = 508 (M + H)⁺ |
| 23 | 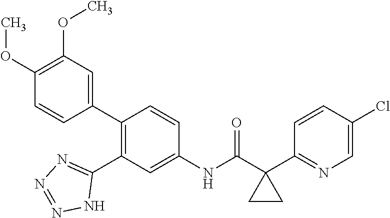 [SMILES: COc1ccc(-c2ccc(NC(=O)C3(c4ccc(Cl)cn4)CC3)cc2-c2nnn[nH]2)cc1OC] | 1-(5- chloropyridin-2- yl)-N-[3′,4′- dimethoxy-2- (1H-tetrazol-5- yl)biphenyl-4- yl]cyclopropane carboxamide | ¹H NMR (400 MHz, DMSO-d6) δ [ppm] 1.38-1.41 (m, 2H), 1.53- 1.56 (m, 2H), 3.58 (s, 3H), 3.74 (s, 3H), 6.57-6.60 (m, 2H), 6.88 (d, 1H), 7.42 (d, 1H), 7.54 (d, 1H), 7.88-7.94 (m, 3H), 8.60 (d, 1H), 10.25 (s, 1H). LCMS (method 1): Rₜ = 1.07 min; MS (ESIpos) m/z = 477 (M + H)⁺ |
| 24 | 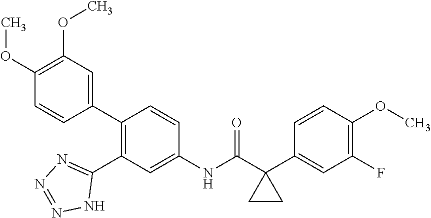 [SMILES: COc1ccc(C2(C(=O)Nc3ccc(-c4ccc(OC)c(OC)c4)c(-c4nnn[nH]4)c3)CC2)cc1F] | N-[3′,4′- dimethoxy-2- (1H-tetrazol-5- yl)biphenyl-4- yl]-1-(3-fluoro- 4- methoxyphenyl) cyclopropane- carboxamide | ¹H NMR (400 MHz, DMSO-d6) δ [ppm] 1.09-1.12 (m, 2H), 1.43- 1.46 (m, 2H), 3.54 (s, 3H), 3.71 (s, 3H), 3.84 (s, 3H), 6.56-6.62 (m, 2H), 6.83 (d, 1H), 7.13-7.27 (m, 3H), 7.43 (d, 1H), 7.70-7.73 (m, 1H), 7.78-7.83 (m, 1H), 9.13 (s, 1H). LCMS (method 3): Rₜ = 1.16 min; MS (ESIpos) m/z = 490 (M + H)⁺ |
| 25 | 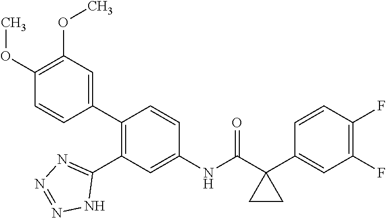 [SMILES: COc1ccc(-c2ccc(NC(=O)C3(c4ccc(F)c(F)c4)CC3)cc2-c2nnn[nH]2)cc1OC] | 1-(3,4- difluorophenyl)- N-[3′,4′- dimethoxy-2- (1H-tetrazol-5- yl)biphenyl-4- yl]cyclopropane- carboxamide | ¹H NMR (400 MHz, DMSO-d6) δ [ppm] 1.16-1.19 (m, 2H), 1.48- 1.51 (m, 2H), 3.56 (s, 3H), 3.73 (s, 3H), 6.55-6.60 (m, 2H), 6.86 (d, 1H), 7.25-7.30 (m, 1H), 7.39- 7.53 (m, 3H), 7.79 (d, 1H), 7.85 (dd, 1H), 9.24 (s, 1H). LCMS (method 3): Rₜ = 1.19 min; MS (ESIpos) m/z = 478 (M + H)⁺ |
| 26 | 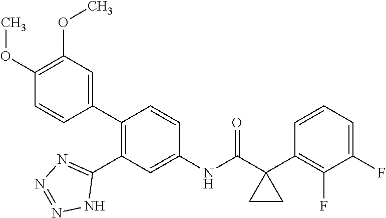 [SMILES: COc1ccc(-c2ccc(NC(=O)C3(c4cccc(F)c4F)CC3)cc2-c2nnn[nH]2)cc1OC] | 1-(2,3- difluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.21-1.23 (m, 2H), 1.62- 1.64 (m, 2H), 3.57 (s, 3H), 3.73 (s, 3H), 6.56-6.59 (m, 2H), 6.87 (d, 1H), 7.20-7.32 (m, 2H), 7.39- 7.46 (m, 1H), 7.50 (d, 1H), 7.78 (d, 1H), 7.86 (dd, 1H), 9.16 (s, 1H). LCMS (method 3): Rₜ = 1.15 min; MS (ESIpos) m/z = 478 (M + H)⁺ |
| 27 | 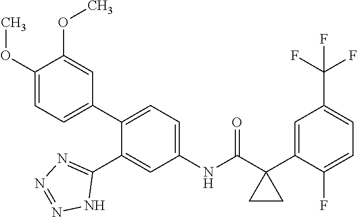 [SMILES: COc1ccc(-c2ccc(NC(=O)C3(c4cc(C(F)(F)F)ccc4F)CC3)cc2-c2nnn[nH]2)cc1OC] | N-[3′,4′- dimethoxy-2- (1H-tetrazol-5- yl)biphenyl-4- yl]-1-[2-fluoro- 5- (trifluoromethyl) phenyl] cyclopropane- carboxamide | ¹H NMR (400 MHz, DMSO-d6) δ [ppm] 1.24-1.29 (m, 2H), 1.62- 1.65 (m, 2H), 3.57 (s, 3H), 3.73 (s, 3H), 6.56-6.60 (m, 2H), 6.86 (d, 1H), 7.44-7.50 (m, 2H), 7.75- 7.89 (m, 4H), 9.23 (s, 1H). LCMS (method 3): Rₜ = 1.25 min; MS (ESIpos) m/z = 528 (M + H)⁺ |
| 28 | 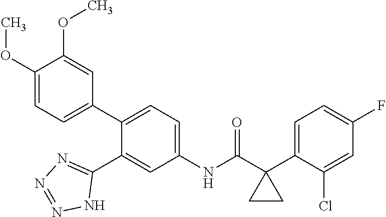 [SMILES: COc1ccc(-c2ccc(NC(=O)C3(c4ccc(F)cc4Cl)CC3)cc2-c2nnn[nH]2)cc1OC] | 1-(2-chloro-4- fluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.14-1.18 (m, 2H), 1.63- 1.66 (m, 2H), 3.56 (s, 3H), 3.72 (s, 3H), 6.56-6.61 (m, 2H), 6.86 (d, 1H), 7.23-7.30 (m, 1H), 7.41- 7.47 (m, 1H), 7.50 (dd, 1H), 7.60 (dd, 1H), 7.71 (s, br, 1H), 7.82 (d, br, 1H), 8.90 (s, 1H). LCMS (method 3): Rₜ = 1.21 min; MS (ESIpos) m/z = 494 (M + H)⁺ |
| 29 | 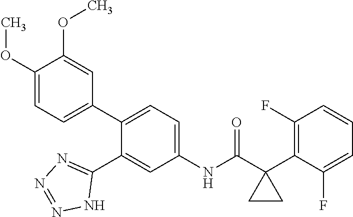 [SMILES: COc1ccc(-c2ccc(NC(=O)C3(c4c(F)cccc4F)CC3)cc2-c2nnn[nH]2)cc1OC] | 1-(2,6- difluorophenyl)- N-[3′,4′- dimethoxy-2- (1H-tetrazol-5- yl)biphenyl-4- yl]cyclopropane carboxamide | ¹H NMR (400 MHz, DMSO-d6) δ [ppm] 1.18-1.21 (m, 2H), 1.72- 1.75 (m, 2H), 3.57 (s, 3H), 3.73 (s, 3H), 6.56-6.59 (m, 2H), 6.87 (d, 1H), 7.11-7.15 (m, 2H), 7.44- 7.51 (m, 2H), 7.78 (d, 1H), 7.87 (dd, 1H), 9.10 (s, 1H). LCMS (method 3): Rₜ = 1.15 min; MS (ESIpos) m/z = 478 (M + H)⁺. |
| 30 | 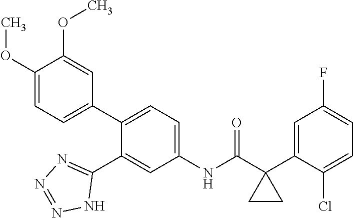 [SMILES: COc1ccc(-c2ccc(NC(=O)C3(c4cc(F)ccc4Cl)CC3)cc2-c2nnn[nH]2)cc1OC] | 1-(2-chloro-5- fluorophenyl)- N-[3′,4′- dimethoxy-2- (1H-tetrazol-5- yl)biphenyl-4- yl]cyclopropane carboxamide | LCMS (method 1): Rₜ = 1.15 min; m/z = 494 (M + H)⁺. |
| 31 | 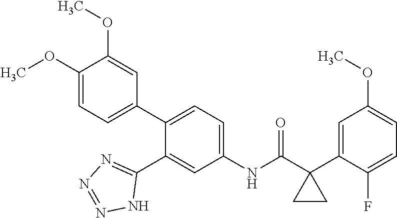 [SMILES: COc1ccc(F)c(C2(C(=O)Nc3ccc(-c4ccc(OC)c(OC)c4)c(-c4nnn[nH]4)c3)CC2)c1] | N-[3′,4′- dimethoxy-2- (1H-tetrazol-5- yl)biphenyl-4- yl]-1-(2-fluoro- 5- methoxyphenyl) cyclopropane- carboxamide | ¹H NMR (400 MHz, DMSO-d6) δ [ppm] 1.15-1.17 (m, 2H), 1.54- 1.57 (m, 2H), 3.54 (s, 3H), 3.72 (s, 3H), 3.78 (s, 3H), 6.56-6.61 (m, 2H), 6.81-6.87 (m, 1H), 6.90-6.94 (m, 1H), 6.99 (dd, 1H), 7.13 (dd, 2H), 7.43 (d, 1H), 7.68-7.72 (m, 1H), 7.80 (d, 1H), 9.02 (s, 1H). LCMS (method 3): Rt = 1.15 min; MS (ESIpos) m/z = 490 (M + H)⁺ |
| 32 | 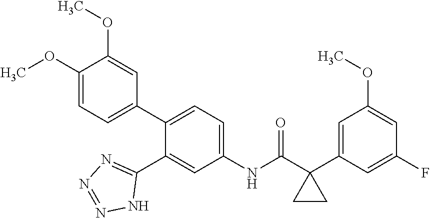 [SMILES: COc1cc(F)cc(C2(C(=O)Nc3ccc(-c4ccc(OC)c(OC)c4)c(-c4nnn[nH]4)c3)CC2)c1] | N-[3′,4′- dimethoxy-2- (1H-tetrazol-5- yl(biphenyl-4- yl]-1-(3-fluoro- 5- methoxyphenyl) cyclopropane- carboxamide | ¹H NMR (400 MHz, DMSO-d6) δ [ppm] 1.17-1.20 (m, 2H), 1.45- 1.48 (m, 2H), 3.58 (s, 3H), 3.73 (s, 3H), 3.78 (s, 3H), 6.55-6.59 (m, 2H), 6.76-6.83 (m, 3H), 6.87 (d, 1H), 7.51 (d, 1H), 7.85 (d, 1H), 7.88 (dd, 1H), 9.40 (s, 1H). LCMS (method 3): Rt = 1.19 min; MS (ESIpos) m/z = 490 (M + H)⁺ |
| 33 | 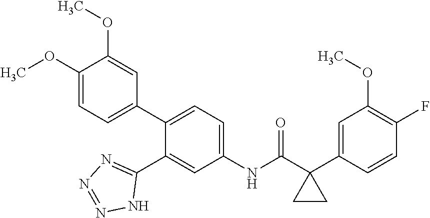 [SMILES: COc1cc(C2(C(=O)Nc3ccc(-c4ccc(OC)c(OC)c4)c(-c4nnn[nH]4)c3)CC2)ccc1F] | N-[3′,4′- dimethoxy-2- (1H-tetrazol-5- yl)biphenyl-4- yl]-1-(4-fluoro- 3- methoxyphenyl) cyclopropane- carboxamide | ¹H NMR (400 MHz, DMSO-d6) δ [ppm] 1.15-1.18 (m, 2H), 1.47- 1.49 (m, 2H), 3.57 (s, 3H), 3.73 (s, 3H), 3.87 (s, 3H), 6.55-6.58 (m, 2H), 6.87 (d, 1H), 6.99-7.02 (m, 1H), 7.17-7.22 (m, 2H), 7.49 (d, 1H), 7.82 (d, 1H), 7.86 (dd, 1H), 9.14 (s, 1H). LCMS (method 1): Rt = 1.13 min; m/z = 490 (M + H)⁺ |
| 34 | 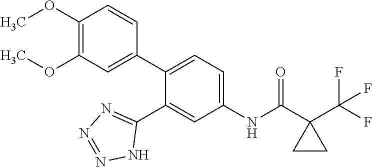 [SMILES: COc1ccc(-c2ccc(NC(=O)C3(C(F)(F)F)CC3)cc2-c2nnn[nH]2)cc1OC] | N-[3′,4′- dimethoxy-2- (1H-tetrazol-5- yl)biphenyl-4- yl]-1- (trifluoromethyl) cyclopropane- carboxamide | ¹H NMR (500 MHz, DMSO-d6) δ [ppm] 9.97 (s, 1H), 7.90 (d, J = 2.2 Hz, 1H), 7.87 (dd, J = 8.5, 2.3 Hz, 1H), 7.56 (d, J = 8.4 Hz, 1H), 6.88 (d, J = 8.0 Hz, 1H), 6.62-6.55 (m, 2H), 3.73 (s, 3H), 3.59 (s, 3H), 1.54-1.48 (m, 2H), 1.37-1.31 (m, 2H). LCMS (Analytical Method D): Rt = 3.79 mins; MS (ESIpos) m/z = 433.95 (M + H)⁺. |
| 35 | 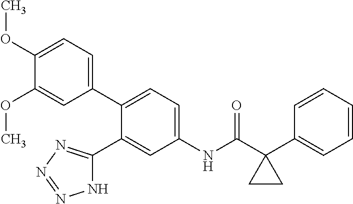 [SMILES: COc1ccc(-c2ccc(NC(=O)C3(c4ccccc4)CC3)cc2-c2nnn[nH]2)cc1OC] | N-[3′,4′- dimethoxy-2- (1H-tetrazol-5- yl)biphenyl-4- yl]-1- phenylcyclo- propane- carboxamide | ¹H NMR (500 MHz, DMSO-d6) δ [ppm] 1.08-1.21 (m, 2H), 1.44- 1.55 (m, 2H), 3.57 (s, 3H), 3.73 (s, 3H), 6.54-6.58 (m, 2H), 6.87 (d, J = 8.2 Hz, 1H), 7.27-7.32 (m, 1H), 7.34-7.43 (m, 4H), 7.50 (d, J = 8.9 Hz, 1H), 7.80- 7.90 (m, 2H), 9.37 (s, 1H). LCMS (Analytical Method F): Rt = 3.25 mins; MS (ESIpos): m/z = 442 (M + H)⁺ |
| 36 | 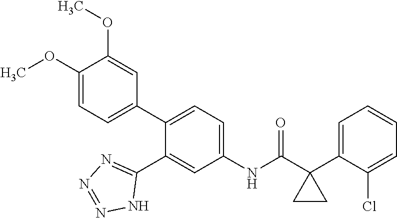 [SMILES: COc1ccc(-c2ccc(NC(=O)C3(c4ccccc4Cl)CC3)cc2-c2nnn[nH]2)cc1OC] | 1-(2- chlorophenyl)- N-[3′,4′- dimethoxy-2- (1H-tetrazol-5- yl)biphenyl-4- yl]cyclopropane carboxamide | ¹H NMR (500 MHz, DMSO-d6) δ [ppm] 1.10-1.23 (m, 2H), 1.59- 1.72 (m, 2H), 3.55 (s, 3H), 3.72 (s, 3H), 6.53-6.62 (m, 2H), 6.84 (d, J = 8.3 Hz, 1H), 7.34-7.41 (m, 2H), 7.43 (d, J = 8.5 Hz, 1H), 7.46-7.52 (m, 1H), 7.54 (dd, J = 6.0, 3.3 Hz, 1H), 7.70 (s, 1H), 7.79 (dd, J = 8.5, 2.0 Hz, 1H), 8.82 (s, 1H). LCMS (Analytical Method D): Rt = 4.12 mins, MS (ESIpos) m/z = 476 (M + H)⁺ |
| 37 | 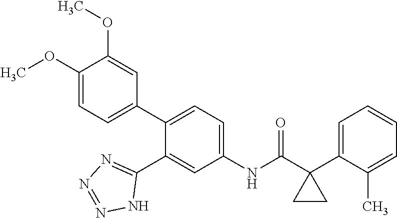 [SMILES: COc1ccc(-c2ccc(NC(=O)C3(c4ccccc4C)CC3)cc2-c2nnn[nH]2)cc1OC] | N-[3′,4′- dimethoxy-2- (1H-tetrazol-5- yl)biphenyl-4- yl]-1-(2- methylphenyl) cyclopropane- carboxamide | ¹H NMR (500 MHz, DMSO-d6) δ [ppm] 1.05-1.12 (m, 2H), 1.55- 1.62 (m, 2H), 2.32 (s, 3H), 3.56 (s, 3H), 3.71 (s, 3H), 6.51-6.59 (m, 2H), 6.85 (d, J = 8.3 Hz, 1H), 7.19-7.27 (m, 3H), 7.37-7.42 (m, 1H), 7.46 (d, J = 8.5 Hz, 1H), 7.74 (d, J = 2.3 Hz, 1H), 7.76- 7.83 (m, 1H). LCMS (Analytical Method F): Rt = 4.35 mins, MS (ESIpos) m/z = 456 (M + H)⁺ |
| 38 | 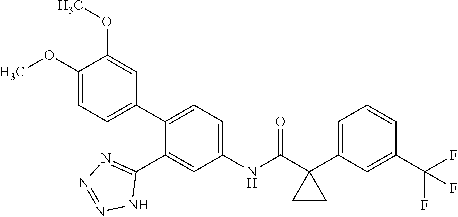 [SMILES: COc1ccc(-c2ccc(NC(=O)C3(c4cccc(C(F)(F)F)c4)CC3)cc2-c2nnn[nH]2)cc1OC] | N-[3′,4′- dimethoxy-2- (1H-tetrazol-5- yl)biphenyl-4- yl]-1-[3- (trifluoromethyl) phenyl] cyclopropane- carboxamide | ¹H NMR (500 MHz, Methanol-d4) δ [ppm] 1.21-1.31 (m, 2H), 1.60- 1.72 (m, 2H), 3.65 (s, 3H), 3.81 (s, 3H), 6.58 (d, J = 2.1 Hz, 1H), 6.67 (dd, J = 8.3, 2.1 Hz, 1H), 6.89 (d, J = 8.3 Hz, 1H), 7.50 (d, J = 8.5 Hz, 1H), 7.56-7.67 (m, 2H), 7.69-7.81 (m, 4H). LCMS (Analytical Method F): Rt = 3.54 mins, MS (ESIpos) m/z = 510 (M + H)⁺. |
| 39 | 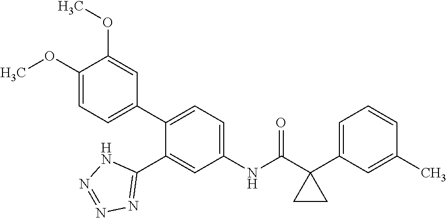 | N-[3′,4′- dimethoxy-2- (1H-tetrazol-5- yl)biphenyl-4- yl]-1-(3- methylphenyl) cyclopropane- carboxamide | ¹H NMR (500 MHz, DMSO-d6) δ [ppm] 9.28 (s, 1H), 7.86-7.81 (m, 2H), 7.48 (d, J = 8.3 Hz, 1H), 7.27-7.16 (m, 3H), 7.10 (d, J = 7.3 Hz, 1H), 6.85 (d, J = 8.3 Hz, 1H), 6.59-6.52 (m, 2H), 3.72 (s, 3H), 3.56 (s, 3H), 2.31 (s, 3H), 1.44 (m, 2H), 1.11 (m, 2H). LCMS (Analytical Method F): Rt = 3.51 min, MS (ESIpos) m/z = 456.2 (M + H)⁺. |
| 40 | 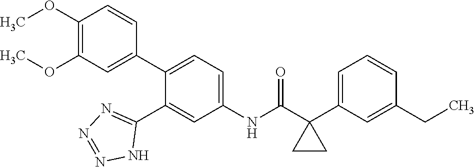 [SMILES: CCc1cccc(C2(C(=O)Nc3ccc(-c4ccc(OC)c(OC)c4)c(-c4nnn[nH]4)c3)CC2)c1] | N-[3′,4′- dimethoxy-2- (1H-tetrazol-5- yl)biphenyl-4- yl]-1-(3- ethylphenyl) cyclopropane- carboxamide | ¹H NMR (500 MHz, DMSO-d6) δ [ppm] 9.34 (s, 1H), 7.88-7.82 (m, 2H), 7.50 (d, J = 8.5 Hz, 1H), 7.30-7.20 (m, 3H), 7.14 (d, J = 7.4 Hz, 1H), 6.87 (d, J = 8.2 Hz, 1H), 6.60-6.53 (m, 2H), 3.73 (s, 3H), 3.57 (s, 3H), 2.62 (q, J = 7.6 Hz, 2H), 1.49-1.42 (m, 2H), 1.19 (t, J = 7.6 Hz, 3H), 1.16- 1.10 (m, 2H). LCMS (Analytical Method D): Rt = 4.50 mins, MS (ESIpos) m/z = 470 (M + H)⁺. |
| 41 | 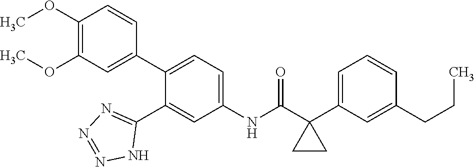 [SMILES: CCCc1cccc(C2(C(=O)Nc3ccc(-c4ccc(OC)c(OC)c4)c(-c4nnn[nH]4)c3)CC2)c1] | N-[3′,4′- dimethoxy-2- (1H-tetrazol-5- yl)biphenyl-4- yl]-1-(3- propylpheny) cyclopropane- carboxamide | ¹H NMR (500 MHz, DMSO-d6) δ [ppm] 9.33 (s, 1H), 7.88-7.80 (m, 2H), 7.50 (d, J = 8.4 Hz, 1H), 7.30-7.24 (m, 1H), 7.24-7.19 (m, 2H), 7.12 (d, J = 7.4 Hz, 1H), 6.87 (d, J = 8.2 Hz, 1H), 6.61- 6.53 (m, 2H), 3.73 (s, 3H), 3.57 (s, 3H), 2.59-2.53 (m, 2H), 1.65- 1.53 (m, 2H), 1.49-1.42 (m, 2H), 1.17-1.10 (m, 2H), 0.89 (t, J = 7.3 Hz, 3H). LCMS (Analytical Method D): Rt = 4.68 mins; MS (ESIpos) m/z = 484.1 (M + H)⁺. |
| 42 | 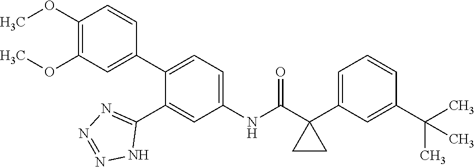 [SMILES: COc1ccc(-c2ccc(NC(=O)C3(c4cccc(C(C)(C)C)c4)CC3)cc2-c2nnn[nH]2)cc1OC] | 1-(3-tert- butylphenyl)-N- [3′,4′- dimethoxy-2- (1H-tetrazol-5- yl)biphenyl-4- yl]cyclopropane carboxamide | ¹H NMR (500 MHz, DMSO-d6) δ [ppm] 9.35 (s, 1H), 7.83-7.74 (m, 2H), 7.47 (d, J = 8.4 Hz, 1H), 7.42 (d, J = 1.7 Hz, 1H), 7.35- 7.25 (m, 2H), 7.22 (dt, J = 7.2, 1.6 Hz, 1H), 6.85 (d, J = 8.4 Hz, 1H), 6.58 (dd, J = 8.2, 2.0 Hz, 1H), 6.55 (d, J = 2.0 Hz, 1H), 3.72 (s, 3H), 3.56 (s, 3H), 1.49- 1.43 (m, 2H), 1.28 (s, 9H), 1.16- 1.11 (m, 2H). LCMS (Analytical Method F): Rt = 3.99 mins; MS (ESIpos) m/z = 498 (M + H)⁺. |
| 43 | 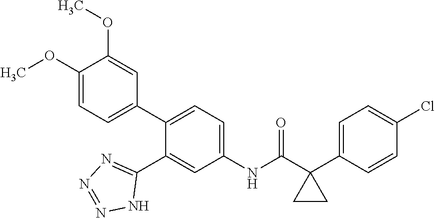 [SMILES: COc1ccc(-c2ccc(NC(=O)C3(c4ccc(Cl)cc4)CC3)cc2-c2nnn[nH]2)cc1OC] | 1-(4- chlorophenyl)- N-[3′,4′- dimethoxy-2- (1H-tetrazol-5- yl)biphenyl-4- yl]cyclopropane carboxamide | ¹H NMR (500 MHz, DMSO-d6) δ [ppm] 1.10-1.18 (m, 2H), 1.46- 1.53 (m, 2H), 3.57 (s, 3H), 3.73 (s, 3H), 6.53-6.61 (m, 2H), 6.86 (d, J = 8.3 Hz, 1H), 7.42 (s, 4H), 7.48 (d, J = 8.5 Hz, 1H), 7.77- 7.88 (m, 2H), 9.35 (s, 1H). LCMS (Analytical Method D): Rt = 3.54 mins; MS (ESIpos): m/z = 476 (M + H)⁺. |
| 44 | 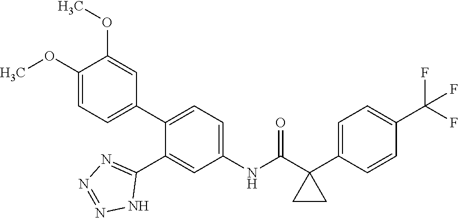 [SMILES: COc1ccc(-c2ccc(NC(=O)C3(c4ccc(C(F)(F)F)cc4)CC3)cc2-c2nnn[nH]2)cc1OC] | N-[3′,4′- dimethoxy-2- (1H-tetrazol-5- yl)biphenyl-4- yl]-1-[4- (trifluoromethyl) phenyl] cyclopropane- carboxamide | ¹H NMR (500 MHz, DMSO-d6) δ [ppm] 1.18-1.29 (m, 2H), 1.48- 1.59 (m, 2H), 3.57 (s, 3H), 3.73 (s, 3H), 6.51-6.61 (m, 2H), 6.87 (d, J = 8.2 Hz, 1H), 7.50 (d, J = 8.3 Hz, 1H), 7.60 (d, J = 8.1 Hz, 2H), 7.71 (d, J = 8.1 Hz, 2H), 7.86 (d, J = 9.4 Hz, 2H), 9.56 (s, 1H). LCMS (Analytical Method D): Rt = 4.48 mins; MS (ESIpos) m/z = 510 (M + H)⁺. |
| 45 | 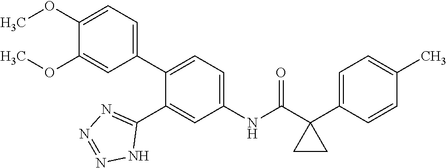 | N-[3′,4′- dimethoxy-2- (1H-tetrazol-5- yl)biphenyl-4- yl]-1-(4- methylphenyl) cyclopropane- carboxamide | ¹H NMR (500 MHz, DMSO) δ [ppm] 9.23 (s, 1H), 7.92-7.79 (m, 2H), 7.49 (d, J = 8.45 Hz, 1H), 7.31 (d, J = 8.08 Hz, 2H), 7.18 (d, J = 8.02 Hz, 2H), 6.87 (d, J = 8.24 Hz, 1H), 6.63-6.51 (m, 2H), 3.73 (s, 3H), 3.58 (s, 3H), 2.31 (s, 3H), 1.45 (m, 2H), 1.10 (m, 2H). LCMS (Analytical Method F): Rt = 3.50 mins; MS (ESIpos) m/z = 456 (M + H)⁺. |
| 46 | 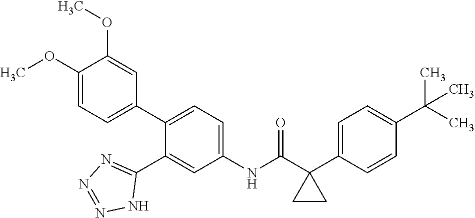 [SMILES: COc1ccc(-c2ccc(NC(=O)C3(c4ccc(C(C)(C)C)cc4)CC3)cc2-c2nnn[nH]2)cc1OC] | 1-(4-tert- butylphenyl)-N- [3′,4′- dimethoxy-2- (1H-tetrazol-5- yl)biphenyl-4- yl]cyclopropane carboxamide | ¹H NMR (500 MHz, DMSO-d6) δ [ppm] 9.42 (s, 1H), 7.91-7.80 (m, 2H), 7.50 (d, J = 9.1 Hz, 1H), 7.40-7.36 (m, 2H), 7.35-7.30 (m, 2H), 6.87 (d, J = 8.2 Hz, 1H), 6.57 (dd, J = 8.1, 2.1 Hz, 1H), 6.55 (d, J = 2.0 Hz, 1H), 3.73 (s, 3H), 3.57 (s, 3H), 1.51-1.40 (m, 2H), 1.28 (s, 9H), 1.16-1.05 (m, 2H). LCMS (Analytical Method D): Rt 4.74 mins; MS (ESIpos) MS (ESIpos) m/z = 498 (M + H)⁺. |
| 47 | 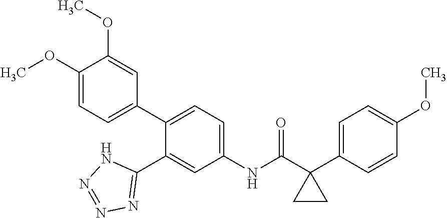 | N-[3′,4′- dimethoxy-2- (1H-tetrazol-5- yl)biphenyl-4- yl]-1-(4- methoxyphenyl) cyclopropane- carboxamide | ¹H NMR (500 MHz, DMSO-d6) δ [ppm] 9.11 (s, 1H), 7.85 (dd, J = 8.5, 2.2 Hz, 1H), 7.82 (d, J = 2.1 Hz, 1H), 7.49 (d, J = 8.5 Hz, 1H), 7.38-7.33 (m, 2H), 6.96-6.91 (m, 2H), 6.86 (d, J = 8.3 Hz, 1H), 6.59-6.53 (m, 2H), 3.76 (s, 3H), 3.72 (s, 3H), 3.57 (s, 3H), 1.44 (m, 2H), 1.08 (m, 2H). LCMS (Analytical Method F): Rt = 3.28 min, MS (ESIpos) MS (ESIpos) m/z = 472.2 (M + H)⁺. |
| 48 | 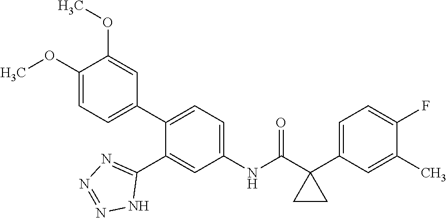 | N-[3′,4′- dimethoxy-2- (1H-tetrazol-5- yl)biphenyl-4- yl]-1-(4-fluoro- 3- methylphenyl) cyclopropane- carboxamide | ¹H NMR (500 MHz, DMSO-d6) δ [ppm] 1.08-1.16 (m, 2H), 1.44- 1.49 (m, 2H), 2.25 (d, J = 1.5 Hz, 3H), 3.58 (s, 3H), 3.73 (s, 3H), 6.53-6.61 (m, 2H), 6.87 (d, J = 8.3 Hz, 1H), 7.09-7.16 (m,1H), 7.25-7.30 (m, 1H), 7.35 (dd, J = 7.5, 1.8 Hz, 1H), 7.49 (d, J = 8.5 Hz, 1H), 7.82 (s, 1H), 7.86 (dd, J = 8.6, 2.1 Hz, 1H), 9.19 (s, 1H). LCMS (Analytical Method D): Rt = 3.50 mins; MS (ESIpos) m/z = 474 (M + H)⁺. |
| 49 | 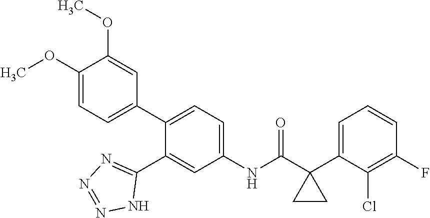 [SMILES: COc1ccc(-c2ccc(NC(=O)C3(c4cccc(F)c4Cl)CC3)cc2-c2nnn[nH]2)cc1OC] | 1-(2-chloro-3- fluorophenyl)- N-[3′,4′- dimethoxy-2- (1H-tetrazol-5- yl)biphenyl-4- yl]cyclopropane carboxamide | ¹H NMR (500 MHz, DMSO-d6) δ [ppm] 1.12-1.23 (m, 2H), 1.62 1.72 (m, 2H), 3.54 (s, 3H), 3.72 (s, 3H), 6.56 (d, J = 2.0 Hz, 1H), 6.60 (dd, J = 8.3, 1.9 Hz, 1H), 6.84 (d, J = 8.3 Hz, 1H), 7.33- 7.53 (m, 4H), 7.67 (s, 1H), 7.72- 7.81 (m, 1H), 8.89 (s, 1H). LCMS (Analytical Method D): Rt = 4.25 mins; MS (ESIpos) m/z = 494 (M + H)⁺. |
| 50 | 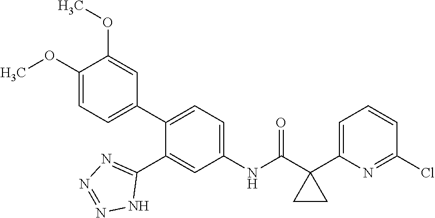 [SMILES: COc1ccc(-c2ccc(NC(=O)C3(c4cccc(Cl)n4)CC3)cc2-c2nnn[nH]2)cc1OC] | 1-(6- chloropyridin-2- yl)-N-[3′,4′- dimethoxy-2- (1H-tetrazol-5- yl)biphenyl-4- yl]cyclopropane carboxamide | ¹H NMR (400 MHz, DMSO-d6) δ [ppm] 1.40-1.42 (m, 2H), 1.54- 1.56 (m, 2H), 3.60 (s, 3H), 3.75 (s, 3H), 6.59-6.61 (m, 2H), 6.90 (d, 1H), 7.40-7.42 (m, 2H), 7.55 (d, 1H), 7.83 (t, 1H), 7.90 (dd, 1H), 7.94 (d, 1H), 10.19 (s, 1H). LCMS (method 1): Rt = 1.06 min; MS (ESIpos) m/z = 477 (M + H)⁺ |
| 51 | 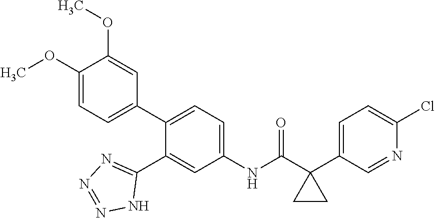 [SMILES: COc1ccc(-c2ccc(NC(=O)C3(c4ccc(Cl)nc4)CC3)cc2-c2nnn[nH]2)cc1OC] | 1-(6- Chloropyridin-3- yl)-N-[3′,4′- dimethoxy-2- (1H-tetrazol-5- yl)biphenyl-4- yl]cyclopropane carboxamide | ¹H NMR (400 MHz, DMSO-d6) δ [ppm] 1.23-1.25 (m, 2H), 1.57- 1.58 (m, 2H), 3.59 (s, 3H), 3.74 (s, 3H), 5.57-6.60 (m, 2H), 6.89 (d, 1H), 7.52 (dd, 2H), 7.81 (d, 1H), 7.88 (dd, 1H), 7.91 (dd, 1H), 8.47 (d, 1H), 9.34 (s, 1H). LCMS (method 1): Rt = 1.00 min; MS (ESIpos) m/z = 477 (M + H)⁺ |
| 52 | 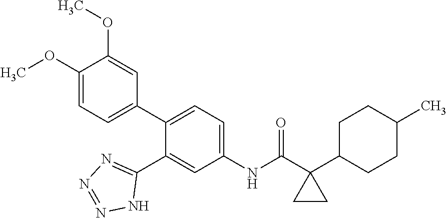 | N-[3′,4′- dimethoxy-2- (1H-tetrazol-5- yl)biphenyl-4- yl]-1-(4- methylcyclohexyl) cyclopropane carboxamide, as a single unknown diastereoisomer | ¹H NMR (500 MHz, Methanol-d4) δ [ppm] 0.74-0.82 (m, 2H), 0.92- 0.99 (m, 5H), 1.37-1.65 (m, 9H), 1.85-1.98 (m, 1H), 3.65 (s, 3H), 3.81 (s, 3H), 6.60 (d, J = 2.1 Hz, 1H), 6.70 (dd, J = 8.3, 2.1 Hz, 1H), 6.89 (d, = 8.3 Hz, 1H), 7.49-7.56 (m, 1H), 7.83-7.90 (m, 2H). LCMS (Analytical Method D): Rt = 4.60 mins; MS (ESIpos): m/z = 462 (M + H)⁺ |
| 53 | 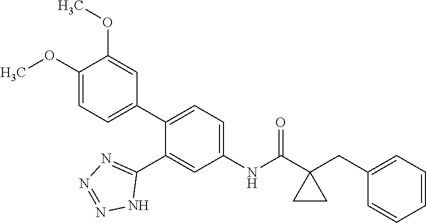 | 1-benzyl-N- [3′,4′- dimethoxy-2- (1H-tetrazol-5- yl)biphenyl-4- yl]cyclopropane carboxamide | ¹H NMR (500 MHz, DMSO-d6) δ [ppm] 9.59 (s, 1H), 7.88 (d, J = 2.2 Hz, 1H), 7.84 (dd, J = 8.5, 2.3 Hz, 1H), 7.49 (d, J = 8.5 Hz, 1H), 7.32-7.23 (m, 4H), 7.21- 7.12 (m, 1H), 6.86 (d, J = 8.2 Hz, 1H), 6.57 (dd, J = 8.2, 2.1 Hz, 1H), 6.55 (d, J = 2.0 Hz, 1H), 3.73 (s, 3H), 3.57 (s, 3H), 3.12 (s, 2H), 1.28-1.13 (m, 2H), 0.89- 0.77 (m, 2H). LCMS (Analytical Method D): Rt = 4.05 mins; MS (ESIpos): m/z = 493 (M + H)⁺. |
| 54 | 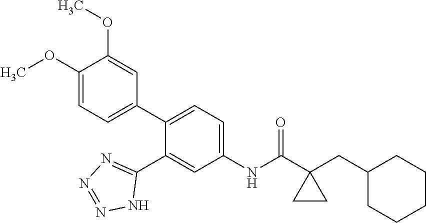 [SMILES: COc1ccc(-c2ccc(NC(=O)C3(CC4CCCCC4)CC3)cc2-c2nnn[nH]2)cc1OC] | 1- (cyclohexyl- methyl)-N-[3′,4′- dimethoxy-2- (1H-tetrazol-5- yl)biphenyl-4- yl]cyclopropane carboxamide | ¹H NMR (500 MHz, DMSO-d6) δ [ppm] 9.51 (s, 1H), 7.93 (d, J = 2.1 Hz, 1H), 7.90 (dd, J = 8.5, 2.2 Hz, 1H), 7.51 (d, J = 8.5 Hz, 1H), 6.88 (d, J = 8.2 Hz, 1H), 6.58 (dd, J = 8.2, 2.0 Hz, 1H), 6.57 (d, J = 1.9 Hz, 1H), 3.73 (s, 3H), 3.58 (s, 3H), 1.76 (d, J = 12.3 Hz, 2H), 1.68-1.60 (m, 4H), 1.60-1.54 (m, 1H), 1.51- 1.39 (m, 1H), 1.21-1.09 (m, 3H), 1.09-1.05 (m, 2H), 0.98- 0.84 (m, 2H), 0.68-0.56 (m, 2H). LCMS (Analytical Method D): Rt = 4.54 mins; MS (ESIpos) m/z = 462 (M + H)⁺. |
| 55 | 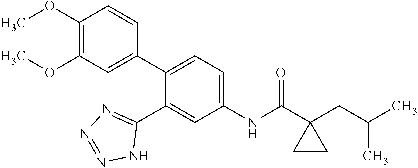 [SMILES: COc1ccc(-c2ccc(NC(=O)C3(CC(C)C)CC3)cc2-c2nnn[nH]2)cc1OC] | N-[3′,4′- dimethoxy-2- (1H-tetrazol-5- yl)biphenyl-4- yl]-1- isobutyl- cyclopropane- carboxamide | ¹H NMR (500 MHz, DMSO-d6) δ [ppm] 9.53 (s, 1H), 7.92 (d, J = 2.1 Hz, 1H), 7.90 (dd, J = 8.5, 2.2 Hz, 1H), 7.51 (d, J = 8.4 Hz, 1H), 6.87 (d, J = 8.2 Hz, 1H), 6.58 (dd, J = 8.2, 2.1 Hz, 1H), 6.57 (d, J = 2.0 Hz, 1H), 3.73 (s, 3H), 3.58 (s, 3H), 1.84-1.70 (m, 1H), 1.63 (d, J = 7.2 Hz, 2H), 1.14-1.02 (m, 2H), 0.91 (d, J = 6.6 Hz, 6H), 0.72-0.55 (m, 2H). LCMS (Analytical Method D): Rt = 4.14 mins; MS (ESIpos) m/z = 422.0 (M + H)⁺. |
| 56 | 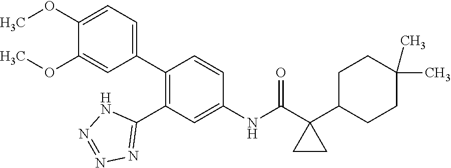 | N-[3′,4′- dimethoxy-2- (1H-tetrazol-5- yl)biphenyl-4- yl]-1-(4,4- dimethyl- cyclohexyl) cyclopropane- carboxamide | ¹H NMR (500 MHz, DMSO-d6) δ 9.51 (s, 1H), 7.84-7.72 (m, 2H), 7.38 (d, J = 8.4 Hz, 1H), 6.82 (d, J = 8.3 Hz, 1H), 6.63 (dd, J = 8.3, 2.0 Hz, 1H), 6.57 (d, J = 2.0 Hz, 1H), 3.71 (s, 3H), 3.52 (s, 3H), 1.63-1.53 (m, 1H), 1.53-1.41 (m, 2H), 1.41-1.31 (m, 2H), 1.31-1.07 (m, 4H), 0.93-0.88 (m, 2H), 0.86 (s, 3H), 0.82 (s, 3H), 0.74-0.64 (m, 2H). LCMS (Analytical Method F): Rt = 3.99 mins; MS (ESIpos) m/z = 476.2 (M + H)⁺. |
| 57 | 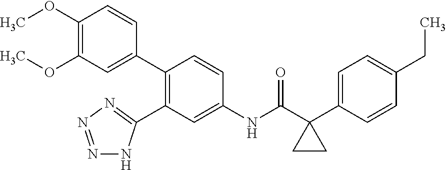 [SMILES: CCc1ccc(C2(C(=O)Nc3ccc(-c4ccc(OC)c(OC)c4)c(-c4nnn[nH]4)c3)CC2)cc1] | N-[3′,4′- dimethoxy-2- (1H-tetrazol-5- yl)biphenyl-4- yl]-1-(4- ethylphenyl) cyclopropane- carboxamide | ¹H NMR (250 MHz, DMSO-d6) δ 1.04-1.25 (m, 5H), 1.37-1.53 (m, 2H), 2.61 (q, J = 7.6 Hz, 2H), 3.58 (s, 3H), 3.73 (s, 3H), 6.43- 6.63 (m, 2H), 6.82-6.93 (m, 1H), 7.12-7.25 (m, 2H), 7.27- 7.37 (m, 2H), 7.44-7.56 (m, 1H), 7.80-7.94 (m, 2H), 9.30 (s, 1H). LCMS (Analytical Method F): Rt = 3.67 mins; MS (ESIpos) m/z = 469.21 (M + H)⁺. |
| 61 | 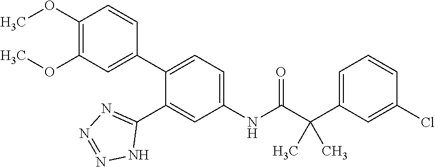 [SMILES: COc1ccc(-c2ccc(NC(=O)C(C)(C)c3cccc(Cl)c3)cc2-c2nnn[nH]2)cc1OC] | 2-(3- chlorophenyl)- N-[3′,4′- dimethoxy-2- (1H-tetrazol-5- yl)biphenyl-4- yl]-2- methyl- propanamide | ¹H NMR (400 MHz, DMSO-d6) δ [ppm] 1.59 (s, 6H), 3.58 (s, 3H), 3.73 (s, 3H), 6.56-6.59 (m, 2H), 6.87 (d, 1H), 7.29-7.42 (m, 4H), 7.52 (d, 1H), 7.89-7.93 (m, 2H), 9.47 (s, 1H). LCMS (method 3): Rt = 1.23 min; MS (ESIpos) m/z = 478 (M + H)⁺ |
| 62 | 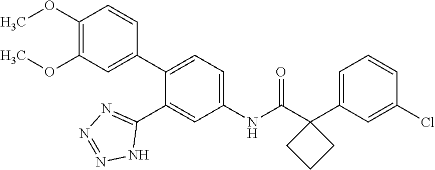 [SMILES: COc1ccc(-c2ccc(NC(=O)C3(c4cccc(Cl)c4)CCC3)cc2-c2nnn[nH]2)cc1OC] | 1-(3- chlorophenyl)- N-[3′,4′- dimethoxy-2- (1H-tetrazol-5- yl)biphenyl-4- yl]cyclobutane- carboxamide | ¹H NMR (400 MHz, DMSO-d6) δ [ppm] 1.18-1.91 (m, 2H), 2.44- 2.54 (m, 2H), 2.83-2.89 (m, 2H), 3.57 (s, 3H), 3.73 (s, 3H), 6.55-6.58 (m, 2H), 6.87 (d, 1H), 7.32-7.35 (m, 1H), 7.38-7.41 (m, 2H), 7.49-7.54 (m, 2H), 7.89-7.91 (m, 2H), 9.78 (s, 1H). LCMS (method 3): Rt = 1.27 min; MS (ESIpos) m/z = 490 (M + H)⁺ |
| 67 | 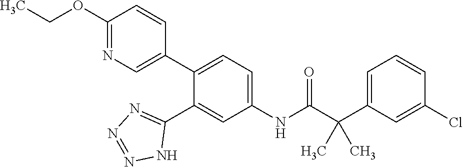 [SMILES: CCOc1ccc(-c2ccc(NC(=O)C(C)(C)c3cccc(Cl)c3)cc2-c2nnn[nH]2)cn1] | 2-(3- chlorophenyl)- N-[4-(6- ethoxypyridin-3- yl)-3-(1H- tetrazol-5- yl)phenyl]-2- methyl- propanamide | ¹H NMR (400 MHz, DMSO-d6) δ [ppm] 1.31 (t, 3H), 1.59 (s, 6H), 4.29 (q, 2H), 6.71 (d, 1H),7.29- 7.43 (m, 5H), 7.50 (d, 1H), 7.90 (dd, 1H), 7.94 (dd, 1H), 8.01 (d, 1H), 9.52 (s, 1H). LCMS (method 3): Rt = 1.27 min; MS (ESIpos) m/z = 463 (M + H)⁺ |
| 68 | 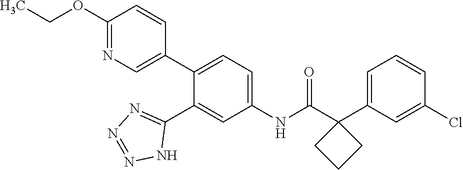 [SMILES: CCOc1ccc(-c2ccc(NC(=O)C3(c4cccc(Cl)c4)CCC3)cc2-c2nnn[nH]2)cn1] | 1-(3- chlorophenyl)- N-[4-(6- ethoxypyridin-3- yl)-3-(1H- tetrazol-5- yl)phenyl] cyclobutane- carboxamide | ¹H NMR (400 MHz, DMSO-d6) δ [ppm] 1.31 (t, 3H), 1.79-1.92 (m, 2H), 1.46-1.52 (m, 2H), 2.83-2.90 (m, 2H), 4.28 (q, 2H), 6.71 (d, 1H), 7.29 (dd, 1H), 7.32- 7.35 (m, 1H), 7.39-7.42 (m, 2H), 7.49 (d, 1H), 7.54 (m, 1H), 7.88 (d, 1H), 7.94 (dd, 1H), 8.01 (d, 1H), 9.84 (s, 1H). LCMS (method 3): Rt = 1.30 min; MS (ESIpos) m/z = 475 (M + H)⁺ |
| 69 | 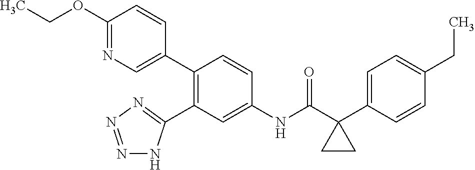 | N-[4-(6- ethoxypyridin-3- yl)-3-(1H- tetrazol-5- yl)phenyl]-1-(4- ethylphenyl) cyclopropane- carboxamide | ¹H NMR (250 MHz, DMSO-d6) δ [ppm] 1.04-1.25 (m, 5H), 1.37- 1.53 (m, 2H), 2.61 (q, J = 7.6 Hz, 2H), 3.58 (s, 3H), 3.73 (s, 3H), 6.43-6.63 (m, 2H), 6.82-6.93 (m, 1H), 7.12-7.25 (m, 2H), 7.27-7.37 (m, 2H), 7.44-7.56 (m, 1H), 7.80-7.94 (m, 2H), 9.30 (s, 1H). LCMS (Analytical Method F): Rt = 3.67 mins; MS (ESIpos) m/z = 469.21 (M + H)⁺. |
| 70 | 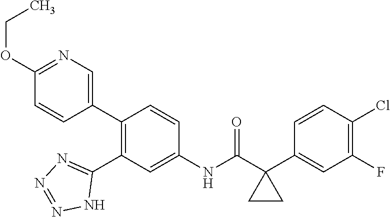 [SMILES: CCOc1ccc(-c2ccc(NC(=O)C3(c4ccc(Cl)c(F)c4)CC3)cc2-c2nnn[nH]2)cn1] | 1-(4-chloro-3- fluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.20-1.23 (m, 2H), 1.31 (t, 3H), 1.50-1.53 (m, 2H), 4.28 (q, 2H), 6.71 (d, 1H), 7.27-7.30 (m, 2H), 7.29-7.32 (m, 2H), 7.45-7.50 (m, 2H), 7.58 (t, 1H), 7.88-7.93 (m, 3H), 9.41 (s, 1H). LCMS (method 1): Rₜ = 1.26 min; MS (ESIpos) m/z = 479 (M + H)⁺ |
| 71 | 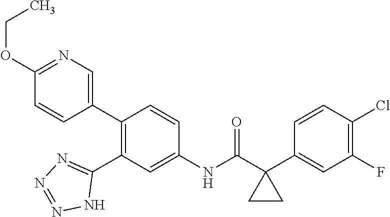 [SMILES: CCOc1ccc(-c2ccc(NC(=O)C3(c4ccc(Cl)c(F)c4)CC3)cc2-c2nnn[nH]2)cn1] | 1-(3-chloro-4- fluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.18-1.21 (m, 2H), 1.31 (t, 3H), 1.49-1.52 (m, 2H), 4.28 (q, 2H), 6.71 (d, 1H), 7.30 (dd, 1H), 7.39-7.50 (m, 3H), 7.64 (dd, 1H), 7.87-7.92 (m, 3H), 9.34 (s, 1H). LCMS (method 1): Rₜ = 1.24 min; MS (ESIpos) m/z = 479 (M + H)⁺ |
| 72 | 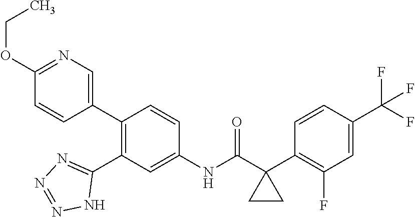 [SMILES: CCOc1ccc(-c2ccc(NC(=O)C3(c4ccc(C(F)(F)F)cc4F)CC3)cc2-c2nnn[nH]2)cn1] | N-[4-(6- ethoxypyridin-3- yl)-3-(1H- tetrazol-5- yl)phenyl]-1-[2- fluoro-4- (trifluoromethyl) phenyl] cyclopropane- carboxamide | ¹H NMR (400 MHz, DMSO-d6) δ [ppm] 1.25-1.28 (m, 2H), 1.31 (t, 3H), 1.64-1.67 (m, 2H), 4.28 (q, 2H), 6.71 (d, 1H), 7.30 (dd, 1H), 7.49 (d, 1H), 7.60-7.63 (m, 1H), 7.68-7.74 (m, 2H), 7.86- 7.90 (m, 3H), 9.27 (s, 1H). LCMS (method 1): Rₜ = 1.27 min; MS (ESIpos) m/z = 513 (M + H)⁺ |
| 73 | 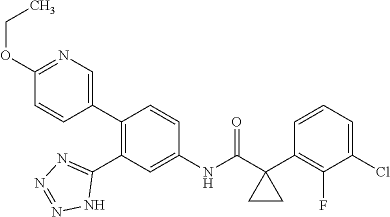 [SMILES: CCOc1ccc(-c2ccc(NC(=O)C3(c4cccc(Cl)c4F)CC3)cc2-c2nnn[nH]2)cn1] | 1-(3-chloro-2- fluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.21-1.24 (m, 2H), 1.31 (t, 3H), 1.62-1.65 (m, 2H), 4.29 (q, 2H), 6.71 (d, 1H), 7.23-7.27 (m, 1H), 7.31 (dd, 1H), 7.44- 7.50 (m, 2H), 7.56-7.60 (m, 1H), 7.86-7.90 (m, 3H), 9.23 (s, 1H). LCMS (method 2): Rₜ = 1.19 min; MS (ESIpos) m/z = 479 (M + H)⁺ |
| 74 | 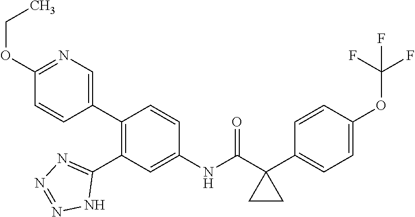 [SMILES: CCOc1ccc(-c2ccc(NC(=O)C3(c4ccc(OC(F)(F)F)cc4)CC3)cc2-c2nnn[nH]2)cn1] | N-[4-(6- ethoxypyridin-3- yl)-3-(1H- tetrazol-5- yl)phenyl]-1-[4- (trifluoromethoxy) phenyl] cyclopropane- carboxamide | ¹H NMR (400 MHz, DMSO-d6) δ [ppm] 1.18-1.21 (m, 2H), 1.31 (t, 3H), 1.50-1.53 (m, 2H), 4.28 (q, 2H), 6.71 (d, 1H), 7.29 (dd, 1H), 7.36 (d, 2H), 7.48 (d, 1H), 7.53 (d, 2H), 7.89-7.90 (m, 2H), 7.95 s, br, 1H), 9.55 (s, 1H). LCMS (method 1): Rₜ = 1.30 min; MS (ESIpos) m/z = 511 (M + H)⁺ |
| 75 | 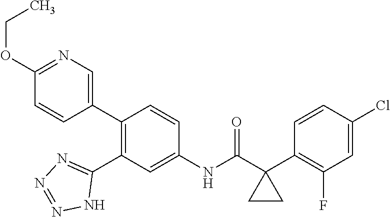 [SMILES: CCOc1ccc(-c2ccc(NC(=O)C3(c4ccc(Cl)cc4F)CC3)cc2-c2nnn[nH]2)cn1] | 1-(4-chloro-2- fluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.17-1.20 (m, 2H), 1.31 (t, 3H), 1.59-1.61 (m, 2H), 4.28 (q, 2H), 6.71 (d, 1H), 7.29-7.33 (m, 2H), 7.44-7.53 (m, 3H), 7.87-7.90 (m, 3H), 9.19 (s, 1H). LCMS (method 1): Rₜ = 1.23 min; MS (ESIpos) m/z = 479 (M + H)⁺ |
| 76 | 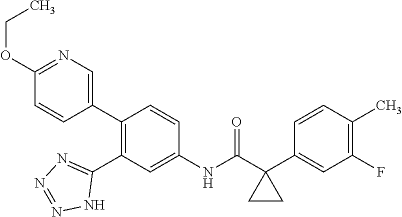 [SMILES: CCOc1ccc(-c2ccc(NC(=O)C3(c4ccc(C)c(F)c4)CC3)cc2-c2nnn[nH]2)cn1] | N-[4-(6- ethoxypyridin-3- yl)-3-(1H- tetrazol-5- yl)phenyl]-1-(3- fluoro-4- methylphenyl) cyclopropane- carboxamide | ¹H NMR (400 MHz, DMSO-d6) δ [ppm] 1.14-1.17 (m, 2H), 1.31 (t, 3H), 1.46-1.49 (m, 2H), 4.24 (d, 3H), 4.28 (q, 2H), 6.71 (d, 1H), 7.14-7.19 (m, 2H), 7.25- 7.31 (m, 2H), 7.48 (d, 1H), 7.89- 7.94 (m, 3H), 9.40 (s, 1H). LCMS (method 2): Rₜ = 1.22 min; MS (ESIpos) m/z = 459 (M + H)⁺ |
| 77 | 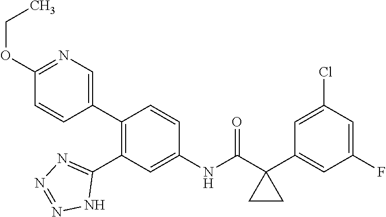 [SMILES: CCOc1ccc(-c2ccc(NC(=O)C3(c4cc(F)cc(Cl)c4)CC3)cc2-c2nnn[nH]2)cn1] | 1-(3-chloro-5- fluorophenyl)- N-[4-(6- ethoxypyridin-3 yl)-3-(1H- tetrazol-5- yl)phenyl] cyclopropane- carboxamide | ¹H NMR (400 MHz, DMSO-d6) δ [ppm] 1.24-1.27 (m, 2H), 1.31 (t, 3H), 1.50-1.53 (m, 2H), 4.29 (q, 2H), 5.71 (d, 1H), 7.25-7.34 (m, 3H), 7.39 (dt, 1H), 7.50 (d, 1H), 7.88-7.95 (m, 3H), 9.51 (s, 1H). LCMS (method 1): Rₜ = 1.25 min; MS (ESIpos) m/z = 479 (M + H)⁺ |
| 78 | 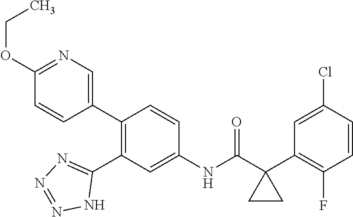 [SMILES: CCOc1ccc(-c2ccc(NC(=O)C3(c4cc(Cl)ccc4F)CC3)cc2-c2nnn[nH]2)cn1] | 1-(5-chloro-2- fluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.22-1.25 (m, 2H), 1.31 (t, 3H), 1.57-1.60 (m, 2H), 4.28 (q, 2H), 6.71 (d, 1H), 7.25-7.31 (m, 2H), 7.44-7.49 (m, 2H), 7.55 (dd, 1H), 7.86-7.90 (m, 3H), 9.28 (s, 1H). LCMS method 1): Rₜ = 1.21 min; MS (ESIpos) m/z = 479 (M + H)⁺ |
| 79 | 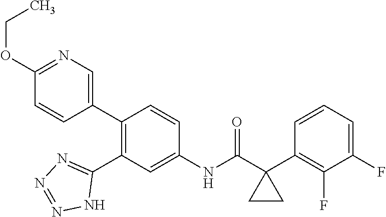 [SMILES: CCOc1ccc(-c2ccc(NC(=O)C3(c4cccc(F)c4F)CC3)cc2-c2nnn[nH]2)cn1] | 1-(2,3- difluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.22-1.25 (m, 2H), 1.31 (t, 3H), 1.62-1.65 (m, 2H), 4.28 (q, 2H), 6.71 (d, 1H), 7.20-7.26 (m, 1H), 7.29-7.32 (m, 2H), 7.39-7.46 (m, 1H), 7.48 (d, 1H), 7.86-7.90 (m, 3H), 9.20 (s, 1H). LCMS (method 1): Rₜ = 1.15 min; MS (ESIpos) m/z = 463 (M + H)⁺ |
| 80 | 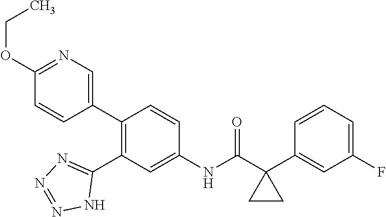 [SMILES: CCOc1ccc(-c2ccc(NC(=O)C3(c4cccc(F)c4)CC3)cc2-c2nnn[nH]2)cn1] | N-[4-(6- ethoxypyridin-3- yl)-3-(1H- tetrazol-5- yl)phenyl]-1-(3- fluorophenyl) cyclopropane- carboxamide | ¹H NMR (400 MHz, DMSO-d6) δ [ppm] 1.19-1.22 (m, 2H), 1.31 (t, 3H), 1.49-1.51 (m, 2H), 4.29 (q, 2H), 6.71 (d, 1H), 7.11-7.16 (m, 1H), 7.21-7.27 (m, 2H), 7.30 (dd, 1H), 7.38-7.44 (m, 1H), 7.49 (d, 1H), 7.88-7.96 (m, 3H), 9.52 (s, 1H). LCMS (method 2): Rₜ = 1.14 min; MS (ESIpos) m/z = 445 (M + H)⁺ |
| 81 | 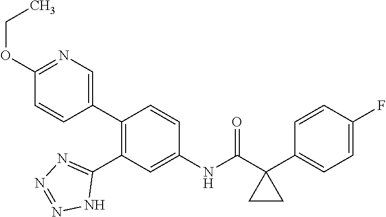 [SMILES: CCOc1ccc(-c2ccc(NC(=O)C3(c4ccc(F)cc4)CC3)cc2-c2nnn[nH]2)cn1] | N-[4-(6- ethoxypyridin-3- yl)-3-(1H- tetrazol-5- yl)phenyl]-1-(4- fluorophenyl) cyclopropane- carboxamide | ¹H NMR (400 MHz, DMSO-d6) δ [ppm] 1.13-1.16 (m, 2H), 1.31 (t, 3H), 1.48-1.51 (m, 2H), 4.28 (q, 2H), 6.70 (d, 1H), 7.18-7.22 (m, 2H), 7.30 (dd, 1H),7.45- 7.49 (m, 3H), 7.88-7.94 (m, 3H), 9.34 (s, 1H). LCMS (method 2): Rₜ = 1.22 min; MS (ESIpos) m/z = 445 (M + H)⁺ |
| 82 | 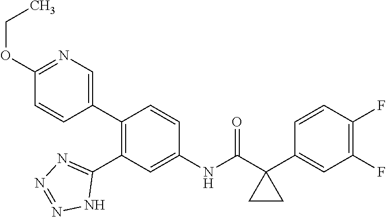 [SMILES: CCOc1ccc(-c2ccc(NC(=O)C3(c4ccc(F)c(F)c4)CC3)cc2-c2nnn[nH]2)cn1] | 1-(3,4- difluorophenyl)- N-[4-(6- ethoxypyridin-3- yl)-3-(1H- tetrazol-5- yl)phenyl] cyclopropane- carboxamide | ¹H NMR (500 MHz, DMSO-d6) δ [ppm] 9.30 (s, 1H), 8.01-7.85 (m, 3H), 7.56-7.46 (m, 2H), 7.46-7.37 (m, 1H), 7.35-7.19 (m, 2H), 6.71 (d, J = 8.5 Hz, 1H), 4.29 (q, J = 7.0 Hz, 2H), 1.55- 1.47 (m, 2H), 1.31 (t, J = 7.0 Hz, 3H), 1.23-1.13 (m, 2H) LCMS (Analytical Method F): Rₜ = 3.45 mins; MS (ESIpos) m/z = 463 (M + H)⁺ |
| 83 | 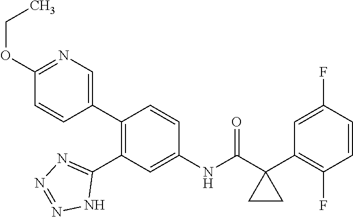 [SMILES: CCOc1ccc(-c2ccc(NC(=O)C3(c4cc(F)ccc4F)CC3)cc2-c2nnn[nH]2)cn1] | 1-(2,5- difluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.21-1.24 (m, 2H), 1.31 (t, 3H), 1.57-1.60 (m, 2H), 4.28 (q, 2H), 6.70 (d, 1H), 7.21-7.31 (m, 3H), 7.37-7.38 (m, 1H), 7.48 (d, 1H), 7.86-7.89 (m, 3H), 9.23 (s, 1H). LCMS (method 1): Rₜ = 1.15 min; MS (ESIpos) m/z = 463 (M + H)⁺ |
| 84 | 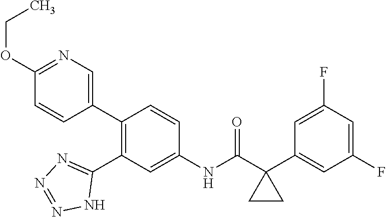 [SMILES: CCOc1ccc(-c2ccc(NC(=O)C3(c4cc(F)cc(F)c4)CC3)cc2-c2nnn[nH]2)cn1] | 1-(3,5- difluorophenyl)- N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.23-1.25 (m, 2H), 1.31 (t, 3H), 1.50-1.53 (m, 2H), 4.29 (q, 2H), 6.71 (d, 1H), 7.12-7.22 (m, 3H), 7.31 (dd, 1H), 7.50 (d, 1H), 7.89-7.95 (m, 3H), 9.50 (s, 1H). LCMS (method 2): Rₜ = 1.17 min; MS (ESIpos) m/z = 463 (M + H)⁺ |
| 85 | 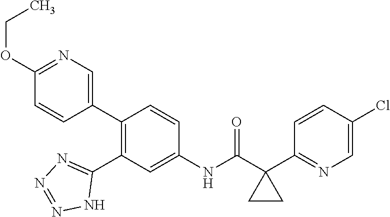 [SMILES: CCOc1ccc(-c2ccc(NC(=O)C3(c4ccc(Cl)cn4)CC3)cc2-c2nnn[nH]2)cn1] | 1-(5- chloropyridin-2- yl)-N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.31 (t, 3H), 1.39-1.42 (m, 2H), 1.54-1.56 (m, 2H), 4.29 (q, 2H), 6.72 (d, 1H), 7.31 (dd, 1H), 7.42 (dd, 1H), 7.88- 7.94 (m, 3H), 8.04 (d, 1H), 8.60 (dd, 1H), 10.30 (s, 1H). LCMS (method 1): Rₜ = 1.11 min; MS (ESIpos) m/z = 462 (M + H)⁺ |
| 86 | 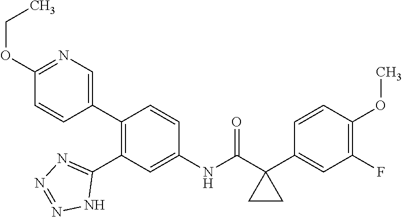 [SMILES: CCOc1ccc(-c2ccc(NC(=O)C3(c4ccc(OC)c(F)c4)CC3)cc2-c2nnn[nH]2)cn1] | N-[4-(6- ethoxypyridin-3- yl)-3-(1H- tetrazol-5- yl)phenyl]-1-(3- fluoro-4- methoxyphenyl) cyclopropane- carboxamide | ¹H NMR (400 MHz, DMSO-d6) δ [ppm] 1.12-1.14 (m, 2H), 1.31 (t, 3H), 1.45-1.47 (m, 2H), 3.85 (s, 3H), 4.28 (q, 2H), 6.70 (d, 1H), 7.13-7.31 (m, 4H), 7.48 (d, 1H), 7.89-7.92 (m, 3H), 9.25 (s, 1H). LCMS (method 1): Rₜ = 1.15 min; MS (ESIpos) m/z = 475 (M + H)⁺ |
| 87 | 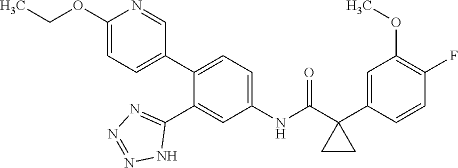 [SMILES: CCOc1ccc(-c2ccc(NC(=O)C3(c4ccc(F)c(OC)c4)CC3)cc2-c2nnn[nH]2)cn1] | N-[4-(6- ethoxypyridin-3- yl)-3-(1H- tetrazol-5- yl)phenyl]-1-(4- fluoro-3- methoxyphenyl) cyclopropane- carboxamide | ¹H NMR (400 MHz, DMSO-d6) δ [ppm] 1.16-1.19 (m, 2H), 1.30 (t, 3H), 1.47-1.50 (m, 2H), 3.87 (s, 3H), 4.28 (q, 2H), 7.00 (d, 1H), 6.99-7.03 (m, 1H), 7.17- 7.22 (m, 2H), 7.29 (dd, 1H), 7.47 (d, 1H), 7.87-7.92 (m, 3H), 9.19 (s, 1H). LCMS (method 1): Rt = 1.17 min; MS (ESIpos) m/z = 475 (M + H)⁺ |
| 88 | 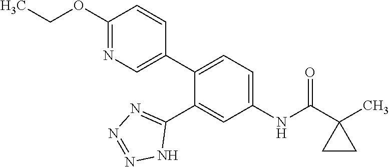 [SMILES: CCOc1ccc(-c2ccc(NC(=O)C3(C)CC3)cc2-c2nnn[nH]2)cn1] | N-[4-(6- ethoxypyridin-3- yl)-3-(1H- tetrazol-5- yl)phenyl]-1- methyl- cyclopropane- carboxamide | ¹H NMR (500 MHz, DMSO-d6) δ [ppm] 9.47 (s, 1H), 8.05 (d, J = 2.1 Hz, 1H), 7.94 (dd, J = 8.5, 2.3 Hz, 1H), 7.92-7.88 (m, 1H), 7.50 (d, J = 8.5 Hz, 1H), 7.31 (dd, J = 8.6, 2.6 Hz, 1H), 6.73- 6.68 (m, 1H), 4.29 (q, J = 7.0 Hz, 2H), 1.43 (s, 3H), 1.31 (t, J = 7.0 Hz, 3H), 1.15-1.09 (m, 2H), 0.72-0.64 (m, 2H). LCMS (Analytical Method D): Rt = 3.77 mins; MS (ESIpos) m/z = 365 (M + H)⁺. |
| 89 | 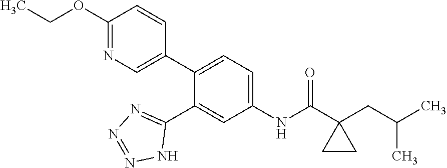 [SMILES: CCOc1ccc(-c2ccc(NC(=O)C3(CC(C)C)CC3)cc2-c2nnn[nH]2)cn1] | N-[4-(6- ethoxypyridin-3- yl)-3-(1H- tetrazol-5- yl)phenyl]-1- isobuty-l cyclopropane- carboxamide | ¹H NMR (500 MHz, DMSO-d6) δ [ppm] 9.68 (s, 1H), 8.04 (d, J = 1.9 Hz, 1H), 7.93 (d, J = 2.2 Hz, 1H), 7.91 (d, J = 2.4 Hz, 1H), 7.50 (d, J = 8.5 Hz, 1H), 7.31 (dd, J = 8.6, 2.5 Hz, 1H), 6.71 (d, J = 8.6 Hz, 1H), 4.29 (q, J = 7.0 Hz, 2H), 1.85-1.72 (m, 1H), 1.64 (d, J = 7.2 Hz, 2H), 1.32 (t, J = 7.0 Hz, 3H), 1.13-1.04 (m, 2H), 0.92 (d, J = 6.6 Hz, 6H), 0.69-0.60 (m, 2H). LCMS (Analytical Method D): Rt = 4.23 mins; MS (ESIpos) m/z = 407.15 (M + H)⁺. |
| 90 | 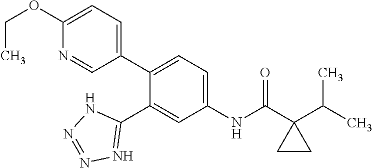 [SMILES: CCOc1ccc(-c2ccc(NC(=O)C3(C(C)C)CC3)cc2-c2nnn[nH]2)cn1] | N-[4-(6- ethoxypyridin-3- yl)-3-(1H- tetrazol-5- yl)phenyl]-1- isopropyl- cyclopropane- carboxamide | ¹H NMR (500 MHz, DMSO-d6) δ [ppm] 9.68 (s, 1H), 8.05 (s, 1H), 7.99-7.84 (m, 2H), 7.49 (d, J = 8.4 Hz, 1H), 7.30 (dd, J = 8.5, 2.3 Hz, 1H), 6.71 (d, J = 8.5 Hz, 1H), 4.29 (q, J = 7.0 Hz, 2H), 2.15-2.00 (m, 1H), 1.31 (t, J = 7.0 Hz, 3H), 0.97-0.87 (m, 8H), 0.75-0.63 (m, 2H). LCMS (Analytical Method F): Rt = 3.06 mins; MS (ESIpos) m/z = 393 (M + H)⁺. |
| 91 | 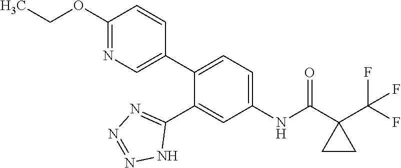 [SMILES: CCOc1ccc(-c2ccc(NC(=O)C3(C(F)(F)F)CC3)cc2-c2nnn[nH]2)cn1] | N-[4-(6- ethoxypyridin-3- yl)-3-(1H- tetrazol-5- yl)phenyl]-1- (trifluoromethyl) cyclopropane- carboxamide | ¹H NMR (500 MHz, DMSO-d6) δ [ppm] 10.03 (s, 1H), 8.01 (d, J = 2.1 Hz, 1H), 7.91 (d, J = 2.6 Hz, 1H), 7.90-7.88 (m, 1H), 7.54 (d, J = 8.5 Hz, 1H), 7.32 (dd, J = 8.6, 2.5 Hz, 1H), 6.71 (d, J = 8.5 Hz, 1H), 4.29 (q, J = 7.0 Hz, 2H), 1.56-1.48 (m, 2H), 1.38-1.33 (m, 2H), 1.31 (t, J = 7.0 Hz, 3H). LCMS (Analytical Method D): Rt = 3.86 mins; MS (ESIpos) m/z = 418.95 (M + H)⁺. |
| 92 | 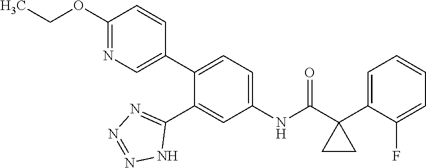 [SMILES: CCOc1ccc(-c2ccc(NC(=O)C3(c4ccccc4F)CC3)cc2-c2nnn[nH]2)cn1] | N-[4-(6- ethoxypyridin-3- yl)-3-(1H- tetrazol-5- yl)phenyl]-1-(2- fluorophenyl) cyclopropane- carboxamide | ¹H NMR (500 MHz, DMSO-d6) δ [ppm] 9.12 (s, 1H), 7.97-7.80 (m, 3H), 7.51-7.44 (m, 2H), 7.43-7.36 (m, 1H), 7.29 (m, 1H), 7.25-7.17 (m, 2H), 6.72- 6.67 (m, 1H), 4.28 (q, J = 7.0 Hz, 2H), 1.63-1.55 (m, 2H), 1.30 (t, J = 7.0 Hz, 3H), 1.21-1.13 (m, 2H). LCMS (Analytical Method D): Rt = 4.16 mins; MS (ESIpos) m/z = 445.0 (M + H)⁺. |
| 93 | 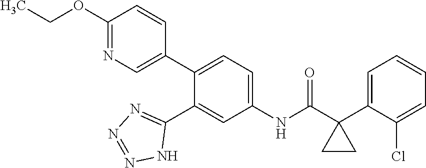 [SMILES: CCOc1ccc(-c2ccc(NC(=O)C3(c4ccccc4Cl)CC3)cc2-c2nnn[nH]2)cn1] | 1-(2- chlorophenyl)- N-[4-(6- ethoxypyridin-3- yl)-3-(1H- tetrazol-5- yl)phenyl] cyclopropane- carboxamide | ¹H NMR (500 MHz, DMSO-d6) δ [ppm] 8.92 (s, 1H), 7.92-7.80 (m, 3H), 7.57-7.52 (m, 1H), 7.51-7.47 (m, 1H), 7.45 (d, J = 8.3 Hz, 1H), 7.41-7.36 (m, 2H), 7.29 (m, 1H), 6.69 (d, J = 8.6 Hz, 1H), 4.28 (q, J = 7.0 Hz, 2H), 1.75-1.56 (m, 2H), 1.30 (t, J = 7.0 Hz, 3H), 1.23-1.09 (m, 2H). LCMS (Analytical Method D): Rt = 4.28 mins; MS (ESIpos) m/z = 461.1 (M + H)⁺. |
| 94 | 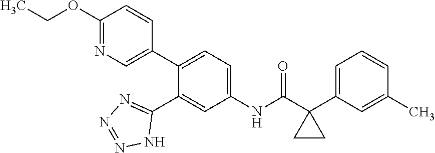 [SMILES: CCOc1ccc(-c2ccc(NC(=O)C3(c4cccc(C)c4)CC3)cc2-c2nnn[nH]2)cn1] | N-[4-(6- ethoxypyridin-3- yl)-3-(1H- tetrazol-5- yl)phenyl]-1-(3- methylphenyl) cyclopropane- carboxamide | ¹H NMR (500 MHz, DMSO-d6) δ [ppm] 9.35 (s, 1H), 7.97-7.93 (m, 1H), 7.90-7.85 (m, 2H), 7.47 (d, J = 8.5 Hz, 1H), 7.29 (dd, J = 8.6, 2.5 Hz, 1H), 7.27- 7.18 (m, 3H), 7.13-7.08 (m, 1H), 6.70 (d, J = 8.6 Hz, 1H), 4.28 (q, J = 7.0 Hz, 2H), 1.48- 1.43 (m, 2H), 1.30 (t, J = 7.0 Hz, 3H), 1.15-1.10 (m, 2H). LCMS (Analytical Method D): Rt = 4.39 mins; MS (ESIpos) m/z = 441.1 (M + H)⁺. |
| 95 | 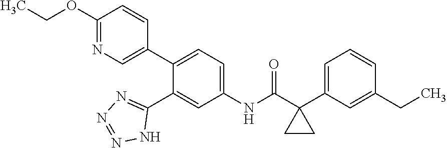 [SMILES: CCOc1ccc(-c2ccc(NC(=O)C3(c4cccc(CC)c4)CC3)cc2-c2nnn[nH]2)cn1] | N-[4-(6- ethoxypyridin-3- yl)-3-(1H- tetrazol-5- yl)phenyl]-1-(3- ethylphenyl) cyclopropane- carboxamide | ¹H NMR (500 MHz, DMSO-d6) δ [ppm] 9.39 (s, 1H), 7.95 (d, J = 1.9 Hz, 1H), 7.88 (d, J = 2.4 Hz, 1H), 7.86 (m, 1H), 7.47 (d, J = 8.5 Hz, 1H), 7.32-7.20 (m, 4H), 7.14 (d, J = 7.5 Hz, 1H), 6.69 (d, J = 8.6 Hz, 1H), 4.28 (q, J = 7.0 Hz, 2H), 2.62 (q, J = 7.5 Hz, 2H), 1.48-1.43 (m, 2H), 1.30 (t, J = 7.0 Hz, 3H), 1.19 (t, J = 7.6 Hz, 3H), 1.16-1.12 (m, 2H). LCMS (Analytical Method D): Rt = 4.64 mins; MS (ESIpos) m/z = 455.1 (M + H)⁺. |
| 96 | 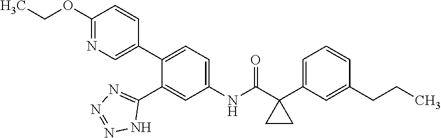 [SMILES: CCCc1cccc(C2(C(=O)Nc3ccc(-c4ccc(OCC)nc4)c(-c4nnn[nH]4)c3)CC2)c1] | N-[4-(6- ethoxypyridin-3- yl)-3-(1H- tetrazol-5- yl)phenyl]-1-(3- propylphenyl) cyclopropane- carboxamide | ¹H NMR (500 MHz, DMSO-d6) δ [ppm] 9.38 (s, 1H), 7.95 (d, J = 2.1 Hz, 1H), 7.88 (d, J = 2.4 Hz, 1H), 7.85 (dd, J = 8.5, 2.2 Hz, 1H), 7.47 (d, J = 8.5 Hz, 1H), 7.32-7.24 (m, 2H), 7.24-7.19 (m, 2H), 7.12 (d, J = 7.4 Hz, 1H), 6.69 (d, J = 8.6 Hz, 1H), 4.28 (q, J = 7.0 Hz, 2H), 2.58-2.53 (m, 2H), 1.65-1.53 (m, 2H), 1.50- 1.41 (m, 2H), 1.30 (t, J = 7.0 Hz, 3H), 1.20-1.08 (m, 2H), 0.90 (t, J = 7.3 Hz, 3H). LCMS (Analytical Method D): Rt = 4.82 mins; MS (ESIpos) m/z = 469.1 (M + H)⁺. |
| 97 | 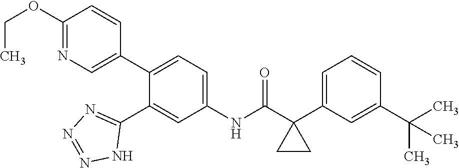 [SMILES: CCOc1ccc(-c2ccc(NC(=O)C3(c4cccc(C(C)(C)C)c4)CC3)cc2-c2nnn[nH]2)cn1] | 1-(3-tert- butylphenyl)-N- [4-(6- ethoxypyridin-3- yl)-3-(1H- tetrazol-5- yl)phenyl] cyclopropane- carboxamide | ¹H NMR (250 MHz, Methanol-d4) δ [ppm] 7.84 (s, 2H), 7.67 (dd, J = 8.4, 2.1 Hz, 1H), 7.56 (s, 1H), 7.52-7.26 (m, 5H), 6.69 (d, J = 8.6 Hz, 1H), 4.29 (q, J = 7.0 Hz, 2H), 1.66-1.54 (m, 2H), 1.42- 1.29 (m, 12H), 1.27-1.16 (m, 2H). LCMS (Analytical Method F): Rt = 4.09 mins; MS (ESIpos) m/z = 483 (M + H)⁺. |
| 98 | 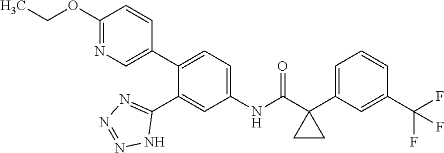 [SMILES: CCOc1ccc(-c2ccc(NC(=O)C3(c4cccc(C(F)(F)F)c4)CC3)cc2-c2nnn[nH]2)cn1] | N-[4-(6- ethoxypyridin-3- yl)-3-(1H- tetrazol-5- yl)phenyl]-1-[3- (trifluoromethyl) phenyl] cyclopropane- carboxamide | ¹H NMR (500 MHz, DMSO-d6) δ [ppm] 9.59 (s, 1H), 7.95 (s, 1H), 7.90-7.85 (m, 2H), 7.75-7.69 (m, 2H), 7.66 (d, J = 7.9 Hz, 1H), 7.63-7.58 (m, 1H), 7.49 (d, J = 8.4 Hz, 1H), 7.34-7.25 (m, 1H), 6.70 (d, J = 8.6 Hz, 1H), 4.28 (q, J = 7.0 Hz, 2H), 1.58-1.52 (m, 2H), 1.30 (t, J = 7.0 Hz, 3H), 1.27-1.22 (m, 2H). LCMS (Analytical Method D): Rt = 4.46 mins; MS (ESIpos) m/z = 495.1 (M + H)⁺. |
| 99 | 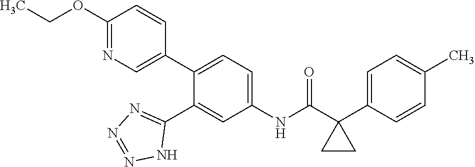 [SMILES: CCOc1ccc(-c2ccc(NC(=O)C3(c4ccc(C)cc4)CC3)cc2-c2nnn[nH]2)cn1] | N-[4-(6- ethoxypyridin-3- yl)-3-(1H- tetrazol-5- yl)phenyl]-1-(4- methylphenyl) cyclopropane- carboxamide | ¹H NMR (500 MHz, DMSO-d6) δ [ppm] 9.28 (s, 1H), 7.93 (s, 1H), 7.90-7.84 (m, 2H), 7.47 (d, J = 8.4 Hz, 1H), 7.33-7.25 (m, 3H), 7.18 (d, J = 7.9 Hz, 2H), 6.74- 6.65 (m, 1H), 4.28 (q, J = 7.0 Hz, 2H), 2.30 (s, 3H), 1.49-1.42 (m, 2H), 1.30 (t, J = 7.0 Hz, 3H), 1.14-1.07 (m, 2H). LCMS (Analytical Method D): Rt = 4.41 mins; MS (ESIpos) m/z = 441.1 (M + H)⁺. |
| 100 | 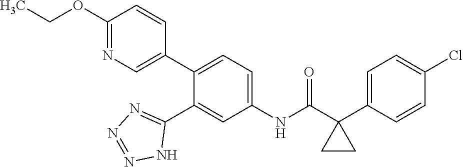 [SMILES: CCOc1ccc(-c2ccc(NC(=O)C3(c4ccc(Cl)cc4)CC3)cc2-c2nnn[nH]2)cn1] | 1-(4- chlorophenyl)- N-[4-(6- ethoxypyridin-3- yl)-3-(1H- tetrazol-5- yl)phenyl] cyclopropane- carboxamide | ¹H NMR (500 MHz, DMSO-d6) δ [ppm] 9.41 (s, 1H), 7.94 (s, 1H), 7.91-7.86 (m, 2H), 7.48 (d, J = 8.4 Hz, 1H), 7.45-7.38 (m, 4H), 7.29 (dd, J = 8.6, 2.3 Hz, 1H), 6.70 (d, J = 8.5 Hz, 1H), 4.28 (q, J = 7.0 Hz, 2H), 1.57-1.43 (m, 2H), 1.30 (t, J = 7.0 Hz, 3H), 1.22-1.07 (m, 2H). LCMS (Analytical Method D): Rt = 4.47 mins; MS (ESIpos) m/z = 461.1 (M + H)⁺. |
| 101 | 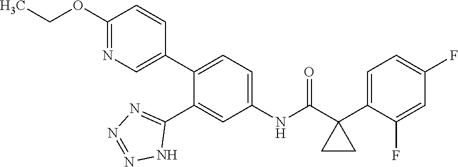 [SMILES: CCOc1ccc(-c2ccc(NC(=O)C3(c4ccc(F)cc4F)CC3)cc2-c2nnn[nH]2)cn1] | 1-(2,4- difluorophenyl)- N-[4-(6- ethoxypyridin-3- yl)-3-(1H- tetrazol-5- yl)phenyl] cyclopropane- carboxamide | ¹H NMR (500 MHz, DMSO-d6) δ [ppm] 9.12 (s, 1H), 7.97-7.81 (m, 3H), 7.57-7.49 (m, 1H), 7.47 (d, J = 8.4 Hz, 1H), 7.30 (dd, J = 8.6, 2.4 Hz, 1H), 7.28- 7.21 (m, 1H), 7.14-7.06 (m, 1H), 6.74-6.64 (m, 1H), 4.28 (q, J = 7.0 Hz, 2H), 1.65-1.54 (m, 2H), 1.30 (t, J = 7.0 Hz, 3H), 1.21-1.11 (m, 2H). LCMS (Analytical Method D): Rt = 4.28 mins; MS (ESIpos) m/z = 463.0 (M + H)⁺. |
| 102 | 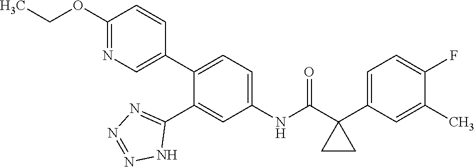 [SMILES: CCOc1ccc(-c2ccc(NC(=O)C3(c4ccc(F)c(C)c4)CC3)cc2-c2nnn[nH]2)cn1] | N-[4-(6- ethoxypyridin-3- yl)-3-(1H- tetrazol-5- yl]phenyl]-1-(4- fluoro-3- methylphenyl) cyclopropane- carboxamide | ¹H NMR (500 MHz, DMSO-d6) δ [ppm] 1.05-1.16 (m, 2H), 1.30 (t, J = 7.0 Hz, 3H), 1.43-1.50 (m, 2H), 2.24 (d, J = 1.5Hz, 3H), 4.28 (q, J = 7.0 Hz, 2H), 6.69 (d, J = 8.6 Hz, 1H), 7.07-7.17 (m, 1H), 7.23-7.32 (m, 2H), 7.34 (dd, J = 7.6, 2.1 Hz, 1H), 7.46 (d, J = 8.5 Hz, 1H), 7.85-7.90 (m, 2H), 7.92 (s, 1H), 9.24 (s, 1H). LCMS (Analytical Method D): Rt = 3.64 mins; MS (ESIpos) m/z = 459.2 (M + H)⁺. |
| 103 | 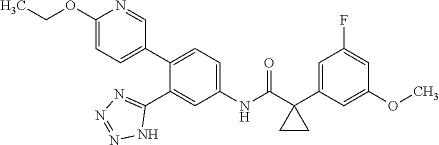 [SMILES: CCOc1ccc(-c2ccc(NC(=O)C3(c4cc(F)cc(OC)c4)CC3)cc2-c2nnn[nH]2)cn1] | N-[4-(6- ethoxypyridin-3- yl)-3-(1H- tetrazol-5- yl)phenyl]-1-(3- fluoro-5- methoxyphenyl) cyclopropane- carboxamide | ¹H NMR (400 MHz, DMSO-d6) δ [ppm] 1.31 (t, 3H), 1.45 (d, 3H), 3.89 (q, 1H), 4.28 (q, 2H), 6.71 (d, 1H), 7.30 (dd, 1H),7.32- 7.41 (m, 3H), 7.46 (m, 1H), 7.51 (d, 1H), 7.84 (dd, 1H), 7.89- 7.91 (m, 2H), 7.95 (d, 1H), 9.45 (s, 1H). LCMS (method 3): Rt = 1.23 min; MS (ESIpos) m/z = 475 (M + H)⁺ |
| 104 | 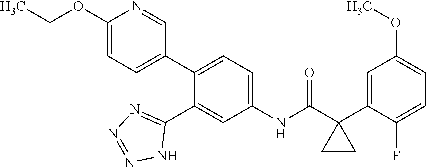 [SMILES: CCOc1ccc(-c2ccc(NC(=O)C3(c4cc(OC)ccc4F)CC3)cc2-c2nnn[nH]2)cn1] | N-[4-(6- ethoxypyridin-3- yl)-3-(1H- tetrazol-5- yl)phenyl]-1-(2- fluoro-5- methoxyphenyl) cyclopropane- carboxamide | ¹H NMR (400 MHz, DMSO-d6) δ [ppm] 1.17-1.20 (m, 2H), 1.30 (t, 3H), 1.55-1.58 (m, 2H), 3.78 (s, 3H), 4.28 (q, 2H), 6.70 (d, 1H), 6.90-6.94 (m, 1H), 7.00 (dd, 1H), 7.11-7.16 (m, 1H), 7.30 (dd, 1H), 7.46 (d, 1H), 7.86- 7.89 (m, 3H), 9.13 (s, 1H). LCMS (method 3): Rt = 1.19 min; MS (ESIpos) m/z = 475 (M + H)⁺ |
| 105 | 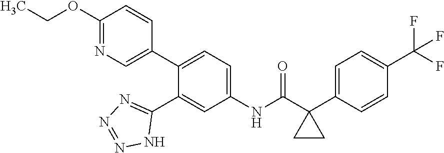 [SMILES: CCOc1ccc(-c2ccc(NC(=O)C3(c4ccc(C(F)(F)F)cc4)CC3)cc2-c2nnn[nH]2)cn1] | N-[4-(6- ethoxypyridin-3- yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.61 (s, 1H), 7.95 (d, J = 2.0 Hz, 1H), 7.91-7.85 (m, 2H), 7.72 (d, J = 8.2 Hz, 2H), 7.60 (d, J = 8.1 Hz, 2H), 7.48 (d, J = 8.5 Hz, 1H), 7.29 (dd, J = 8.6, 2.5 Hz, 1H), 6.70 (d, J = 8.6 Hz, 1H), 4.28 (q, J = 7.0 Hz, 2H), 1.58- 1.54 (m, 2H), 1.30 (t, J = 7.0 Hz, 3H), 1.26-1.22 (m, 2H). LCMS (Analytical Method F): Rt = 3.72 mins, MS (ESIpos): m/z = 495 (M + H)⁺ |
| 106 | 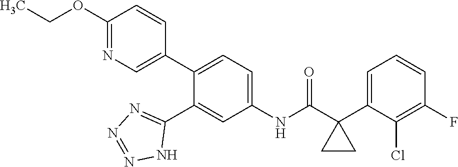 [SMILES: CCOc1ccc(-c2ccc(NC(=O)C3(c4cccc(F)c4Cl)CC3)cc2-c2nnn[nH]2)cn1] | 1-(2-chloro-3- fluorophenyl)- N-[4-(6- ethoxypyridin-3- yl)-3-(1H- tetrazol-5- yl)phenyl] cyclopropane- carboxamide | ¹H NMR (500 MHz, DMSO-d6) δ [ppm] 9.01 (s, 1H), 7.92-7.79 (m, 3H), 7.47 (d, J = 8.9 Hz, 1H), 7.45-7.35 (m, 3H), 7.31 (dd, J = 8.6, 2.5 Hz, 1H), 6.71 (d, J = 8.6 Hz, 1H), 4.28 (q, J = 7.0 Hz, 2H), 1.75-1.59 (m, 2H), 1.30 (t, J = 7.0 Hz, 3H), 1.23-1.14 (m, 2H). LCMS (Analytical Method F): Rt = 3.48 mins; MS (ESIpos): m/z = 479 (M + H)⁺. |
| 107 | 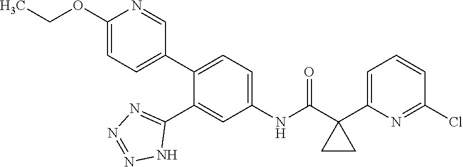 [SMILES: CCOc1ccc(-c2ccc(NC(=O)C3(c4cccc(Cl)n4)CC3)cc2-c2nnn[nH]2)cn1] | 1-(6- chloropyridin-2- yl)-N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.32 (t, 3H), 1.41-1.43 (m, 2H), 1.54-1.56 (m, 2H), 4.31 (q, 2H), 6.73 (d, 1H), 7.33 (dd, 1H), 7.42 (dd, 2H), 7.53 (d, 1H), 7.83 (t, 1H), 7.90-7.92 (m, 2H), 8.05 (d, 1H), 10.25 (s, 1H). LCMS (method 1): Rt = 1.11 min; m/z = 462 (M + H)⁺. |
| 108 | 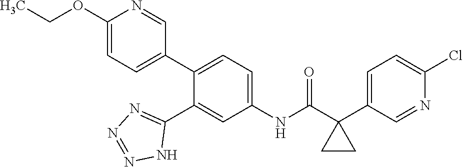 [SMILES: CCOc1ccc(-c2ccc(NC(=O)C3(c4ccc(Cl)nc4)CC3)cc2-c2nnn[nH]2)cn1] | 1-(6- chloropyridin-3- yl)-N-[4-(6- ethoxypyridin-3- yl)-3-(1H- tetrazol-5- yl)phenyl] cyclopropane- carboxamide | ¹H NMR (400 MHz, DMSO-d6) δ [ppm] 1.24-1.26 (m, 2H), 1.32 (t, 3H), 1.57-1.59 (m, 2H), 4.30 (q, 2H), 6.71 (d, 1H), 7.32 (dd, 1H), 7.49 (d, 1H), 7.53 (d, 1H), 7.88-7.92 (m, 4H), 8.47 (d, 1H), 9.38 (s, 1H). LCMS (method 1): Rt = 1.04 min; MS (ESIpos) m/z = 462 (M + H)⁺ |
| 109 | 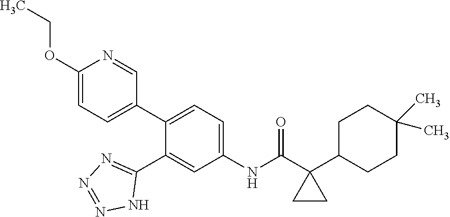 [SMILES: CCOc1ccc(-c2ccc(NC(=O)C3(C4CCC(C)(C)CC4)CC3)cc2-c2nnn[nH]2)cn1] | 1-(4,4- dimethylcyclo- hexyl)-N-[4-(6- ethoxypyridin-3- yl)-3-(1H- tetrazol-5- yl)phenyl] cyclopropane- carboxamide | ¹H NMR (500 MHz, DMSO-d6) δ [ppm] 0.68-0.76 (m, 2H), 0.83 (s, 3H), 0.87 (s, 3H), 0.90-0.94 (m, 2H), 1.09-1.27 (m, 4H), 1.30 (t, J = 7.0 Hz, 3H), 1.33- 1.40 (m, 2H), 1.44-1.53 (m, 2H), 1.55-1.65 (m, 1H), 4.28 (q, J = 7.0 Hz, 2H), 6.70 (d, J = 8.5 Hz, 1H), 7.30 (dd, J = 8.6, 2.5 Hz, 1H), 7.48 (d, J = 8.5 Hz, 1H), 7.79-7.97 (m, 2H), 8.03 (d, J = 1.8 Hz, 1H), 9.67 (s, 1H). LCMS (Analytical Method F): Rt = 4.04 mins; MS (ESIpos): m/z = 461.2 (M + H)⁺. |
| 110 | 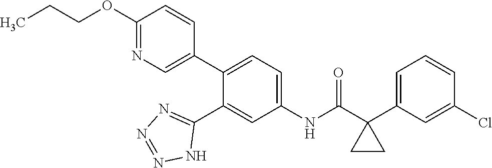 [SMILES: CCCOc1ccc(-c2ccc(NC(=O)C3(c4cccc(Cl)c4)CC3)cc2-c2nnn[nH]2)cn1] | 1-(3- chlorophenyl)- N-[4-(6- propoxypyridin- 3-yl)-3-(1H- tetrazol-5- yl)phenyl] cyclopropane- carboxamide | 1H NMR (500 MHz, DMSO-d6) δ [ppm] 9.52 (s, 1H), 7.95 (d, J = 2.2 Hz, 1H), 7.91-7.85 (m, 2H), 7.48 (d, J = 8.5 Hz, 1H), 7.45- 7.43 (m, 1H), 7.42-7.34 (m, 3H), 7.30 (dd, J = 8.6, 2.6 Hz, 1H), 6.73-6.68 (m, 1H), 4.18 (t, J = 6.7 Hz, 2H), 1.71 (m, 2H), 1.52-1.47 (m, 2H), 1.22-1.17 (m, 2H), 0.95 (t, J = 7.4 Hz, 3H). LCMS (Analytical Method F): Rt = 3.83 mins; MS (ESIpos) m/z = 475, 477 (M + H)+. |
| 111 | 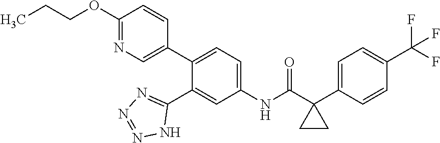 [SMILES: CCCOc1ccc(-c2ccc(NC(=O)C3(c4ccc(C(F)(F)F)cc4)CC3)cc2-c2nnn[nH]2)cn1] | N-[4-(6- propoxypyridin- 3-yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.61 (s, 1H), 7.96 (d, J = 2.0 Hz, 1H), 7.91-7.83 (m, 2H), 7.72 (d, J = 8.2 Hz, 2H), 7.60 (d, J = 8.1 Hz, 2H), 7.48 (d, J = 8.5 Hz, 1H), 7.30 (dd, J = 8.6, 2.6 Hz, 1H), 6.71 (d, J = 8.5 Hz, 1H), 4.18 (t, J = 6.7 Hz, 2H), 1.71 (m, 2H), 1.59-1.53 (m, 2H), 1.27- 1.21 (m, 2H), 0.95 (t, J = 7.4 Hz, 3H). LCMS (Analytical Method F): Rt = 3.94 mins; MS (ESIpos) m/z = 509 (M + H)⁺. |
| 112 | 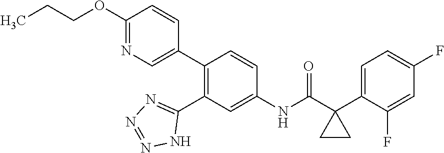 [SMILES: CCCOc1ccc(-c2ccc(NC(=O)C3(c4ccc(F)cc4F)CC3)cc2-c2nnn[nH]2)cn1] | 1-(2,4- difluorophenyl)- N-[4-(6- propoxypyridin- 3-yl)-3-(1H- tetrazol-5- yl)phenyl] cyclopropane- carboxamide | ¹H NMR (500 MHz, DMSO-d6) δ [ppm] 9.12 (s, 1H), 7.88 (dt, J = 5.4, 2.3 Hz, 3H), 7.53 (td, J = 8.7, 6.7 Hz, 1H), 7.47 (d, J = 8.4 Hz, 1H), 7.30 (dd, J = 8.6, 2.5 Hz, 1H), 7.25 (td, J = 10.5, 2.6 Hz, 1H), 7.10 (td, J = 8.5, 2.1 Hz, 1H), 6.74-6.67 (m, 1H), 4.18 (t, J = 6.7 Hz, 2H), 1.71 (m, 2H), 1.62-1.56 (m, 2H), 1.19- 1.13 (m, 2H), 0.95 (t, J = 7.4 Hz, 3H). LCMS (Analytical Method F): Rt = 3.62 mins; MS (ESIpos) m/z = 477 (M + H)⁺. |
| 113 | 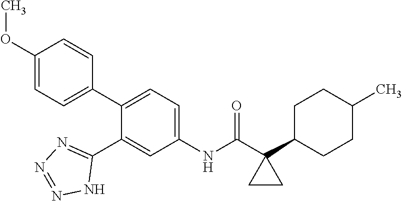 | N-[4′-methoxy- 2-(1H-tetrazol- 5-yl)biphenyl-4- yl]-1-(4- methylcyclo- hexyl) cyclopropane carboxamide, as a single unknown diastereoisomer | ¹H NMR (500 MHz, DMSO-d6) δ [ppm] 0.66-0.74 (m, 2H), 0.85- 0.96 (m, 5H), 1.17-1.37 (m, 2H), 1.37-1.56 (m, 6H), 1.59- 1.71 (m, 1H), 1.83-1.95 (m, 1H), 3.73 (s, 3H), 6.85 (d, J = 8.7 Hz, 2H), 6.96 (d, J = 8.7 Hz, 2H), 7.44 (d, J = 8.6 Hz, 1H), 7.85- 7.91 (m, 1H), 7.92 (s, 1H), 9.63 (s, 1H). LCMS (Analytical Method F): Rt = 3.88 mins; MS (ESIpos) m/z = 432.2 (M + H)⁺. |
| 114 | 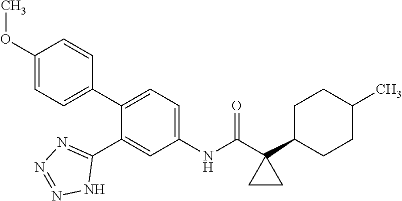 | N-[4′-methoxy- 2-(1H-tetrazol- 5-yl)biphenyl-4- yl]-1-(4- methylcyclo- hexyl) cyclopropane carboxamide, as a 1:1 mixture of cis and trans diastereoisomers | ¹H NMR (500 MHz, DMSO-d6) δ [ppm] 0.65-0.76 (m, 2H), 0.80- 0.98 (m, 6H), 1.01-1.13 (m, 1H), 1.17-1.38 (m, 1.5H), 1.38- 1.57 (m, 3H), 1.58-1.72 (m, 3H), 1.84-1.92 (m, 0.5H), 3.73 (s, 3H), 6.81-6.90 (m, 2H), 6.92- 7.07 (m, 2H), 7.44 (d, J = 8.4 Hz, 1H), 7.89 (dt, J = 8.5, 2.3 Hz, 1H), 7.93 (s, 1H), 9.64 (m, 1H). LCMS (Analytical Method F): Rt = 3.88 and 3.92 mins; MS (ESIpos): m/z = 432.2 (M + H)⁺. |
| 115 | 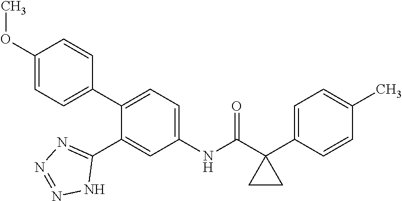 [SMILES: COc1ccc(-c2ccc(NC(=O)C3(c4ccc(C)cc4)CC3)cc2-c2nnn[nH]2)cc1] | N-[4′-methoxy- 2-(1H-tetrazol- 5-yl)biphenyl-4- yl]-1-(4- methylphenyl) cyclopropane- carboxamide | ¹H NMR (500 MHz, DMSO-d6) δ [ppm] 1.06-1.12 (m, 2H), 1.40- 1.50 (m, 2H), 2.31 (s, 3H), 3.74 (s, 3H), 6.83-6.87 (m, 2H), 6.93- 6.98 (m, 2H), 7.18 (d, J = 7.9 Hz, 2H), 7.31 (d, J = 8.1 Hz, 2H), 7.43 (d, J = 8.3 Hz, 1H), 7.80- 7.89 (m, 2H), 9.22 (s, 1H). LCMS (Analytical Method F): Rt = 3.67 mins; MS (ESIpos) m/z = 426.2 (M + H)⁺. |
| 116 | 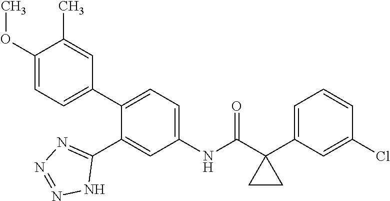 [SMILES: COc1ccc(-c2ccc(NC(=O)C3(c4cccc(Cl)c4)CC3)cc2-c2nnn[nH]2)cc1C] | 1-(3- chlorophenyl)- N-[4′-methoxy- 3′-methyl-2-(1H- tetrazol-5- yl)biphenyl-4- yl]cyclopropane carboxamide | ¹H NMR (500 MHz, DMSO-d6) δ [ppm] 9.44 (s, 1H), 7.89-7.79 (m, 2H), 7.47-7.40 (m, 2H), 7.40-7.32 (m, 3H), 6.88 (d, J = 1.7 Hz, 1H), 6.81 (d, J = 8.5 Hz, 1H), 6.73 (dd, J = 7.6, 2.3 Hz, 1H), 3.74 (s, 3H), 2.06 (s, 3H), 1.48 (m, 2H), 1.18 (m, 2H). LCMS (Analytical Method F): Rt = 4.59 mins; MS (ESIpos) m/z = 460.0 (M + H)⁺. |
| 117 | 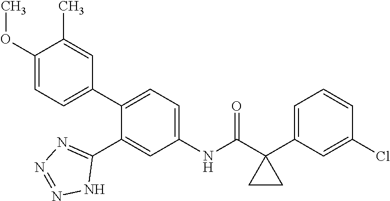 | N-[3′-chloro-4′- methoxy-2-(1H- tetrazol-5- yl)biphenyl-4- yl]-1-(3- chlorophenyl) cyclopropane- carboxamide | ¹H NMR (500 MHz, DMSO-d6) δ [ppm] 9.48 (s, 1H), 7.92-7.81 (m, 2H), 7.48-7.42 (m, 2H), 7.41-7.33 (m, 3H), 7.14 (d, J = 2.2 Hz, 1H), 7.03 (d, J = 8.6 Hz, 1H), 6.87 (d, J = 8.4 Hz, 1H), 3.82 (s, 3H), 1.48 (m, 2H), 1.18 (m, 2H). LCMS (Analytical Method D): Rt = 4.57 mins; MS (ESIpos) m/z = 480.0 (M + H)⁺. |
| 118 | 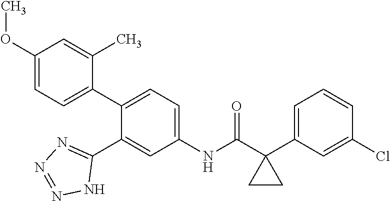 [SMILES: COc1ccc(-c2ccc(NC(=O)C3(c4cccc(Cl)c4)CC3)cc2-c2nnn[nH]2)c(C)c1] | 1-(3- chlorophenyl)- N-[4′-methoxy- 2′-methyl-2-(1H- tetrazol-5- yl)biphenyl-4- yl]cyclopropane carboxamide | ¹H NMR (500 MHz, Methanol-d4) δ [ppm] 1.21-1.30 (m, 2H), 1.59- 1.68 (m, 2H), 1.92 (s, 3H), 3.78 (s, 3H), 6.68-6.75 (m, 2H), 6.93 (d, J = 8.3 Hz, 1H), 7.32 (d, J = 8.4 Hz, 1H), 7.35-7.48 (m, 3H), 7.54 (t, J = 1.7 Hz, 1H), 7.71 (dt, J = 8.4, 2.6 Hz, 1H), 7.87 (t, J = 2.9 Hz, 1H), 8.92 (s, 1H). LCMS (Analytical Method F): Rt = 3.79 mins; MS (ESIpos) m/z = 460.0 (M + H)⁺. |
| 119 | 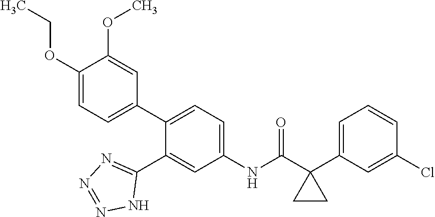 [SMILES: CCOc1ccc(-c2ccc(NC(=O)C3(c4cccc(Cl)c4)CC3)cc2-c2nnn[nH]2)cc1OC] | 1-(3- chlorophenyl)- N-[4′-ethoxy-3′- methoxy-2-(1H- tetrazol-5- yl)biphenyl-4- yl]cyclopropane carboxamide | ¹H NMR (500 MHz, DMSO-d6) δ [ppm] 1.15-1.22 (m, 2H), 1.30 (t, J = 7.0 Hz, 3H), 1.45-1.52 (m, 2H), 3.56 (s, 3H), 3.97 (q, J = 7.0 Hz, 2H), 6.52-6.60 (m, 2H), 6.84 (d, J = 8.3 Hz, 1H), 7.33- 7.41 (m, 3H), 7.41-7.45 (m, 1H), 7.47 (d, J = 8.5 Hz, 1H), 7.80 (d, J = 2.1 Hz, 1H), 7.84 (dd, J = 8.5, 2.2 Hz, 1H), 9.44 (s, 1H). LCMS (Analytical Method F): Rt = 3.65 mins; MS (ESIpos) m/z = 490 (M + H)⁺. |
| 120 | 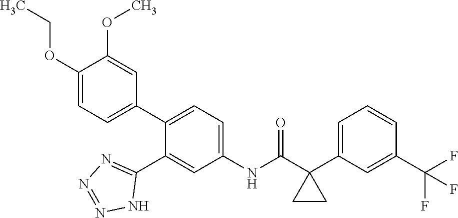 [SMILES: CCOc1ccc(-c2ccc(NC(=O)C3(c4cccc(C(F)(F)F)c4)CC3)cc2-c2nnn[nH]2)cc1OC] | N-[4′-ethoxy-3′- methoxy-2-(1H- tetrazol-5- yl)biphenyl-4- yl]-1-[3- (trifluoromethyl) phenyl] cyclopropane- carboxamide | ¹H NMR (500 MHz, DMSO-d6) δ [ppm] 9.54 (s, 1H), 7.85 (d, J = 8.1 Hz, 2H), 7.75-7.68 (m, 2H), 7.66 (d, J = 7.8 Hz, 1H), 7.60 (t, J = 7.7 Hz, 1H), 7.51 (d, J = 8.1 Hz, 1H), 6.85 (d, J = 8.1 Hz, 1H), 6.59-6.50 (m, 2H), 3.97 (q, J = 7.0 Hz, 2H), 3.57 (s, 3H), 1.60- 1.48 (m, 2H), 1.30 (t, J = 7.0 Hz, 3H), 1.27-1.20 (m, 2H). LCMS (Analytical Method F): Rt = 4.31 mins; MS (ESIpos) m/z = 524 (M + H)⁺. |
| 121 | 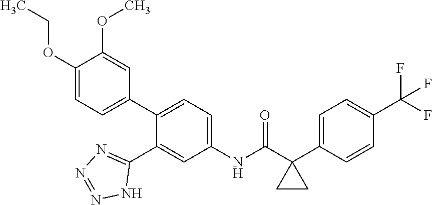 [SMILES: CCOc1ccc(-c2ccc(NC(=O)C3(c4ccc(C(F)(F)F)cc4)CC3)cc2-c2nnn[nH]2)cc1OC] | N-[4′-ethoxy-3′- methoxy-2-(1H- tetrazol-5- yl(biphenyl-4- yl]-1-[4- (trifluoromethyl) phenyl] cyclopropane- carboxamide | ¹H NMR (500 MHz, DMSO-d6) δ [ppm] 9.57 (s, 1H), 7.87 (d, J = 8.8 Hz, 2H), 7.72 (d, J = 8.2 Hz, 2H), 7.61 (d, J = 8.1 Hz, 2H), 7.52 (d, J = 8.3 Hz, 1H), 6.86 (d, J = 8.1 Hz, 1H), 6.59-6.52 (m, 2H), 3.98 (q, J = 7.0 Hz, 2H), 3.58 (s, 3H), 1.61-1.47 (m, 2H), 1.31 (t, J = 7.0 Hz, 3H), 1.27- 1.19 (m, 2H). LCMS (Analytical Method F): Rt = 4.34 mins; MS (ESIpos) m/z = 524 (M + H)⁺. |
| 122 | 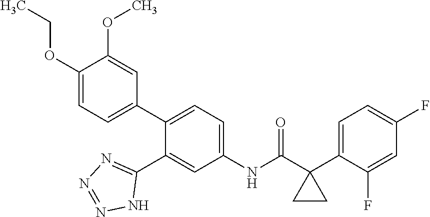 [SMILES: CCOc1ccc(-c2ccc(NC(=O)C3(c4ccc(F)cc4F)CC3)cc2-c2nnn[nH]2)cc1OC] | 1-(2,4- difluorophenyl)- N-[4′-ethoxy-3′- methoxy-2-(1H- tetrazol-5- yl)biphenyl-4- yl]cyclopropane carboxamide | ¹H NMR (500 MHz, DMSO-d6) δ [ppm] 9.07 (s, 1H), 7.86 (d, J = 8.6 Hz, 1H), 7.77 (s, 1H), 7.62- 7.41 (m, 2H), 7.35-7.18 (m, 1H), 7.10 (td, J = 8.6, 2.3 Hz, 1H), 6.85 (d, J = 8.2 Hz, 1H), 6.64-6.51 (m, 2H), 3.98 (q, J = 7.0 Hz, 2H), 3.57 (s, 3H), 1.64- 1.52 (m, 2H), 1.31 (t, J = 7.0 Hz, 3H), 1.21-1.12 (m, 2H). LCMS (Analytical Method F): Rt = 3.44 mins; MS (ESIpos) m/z = 492 (M + H)⁺. |
| 123 | 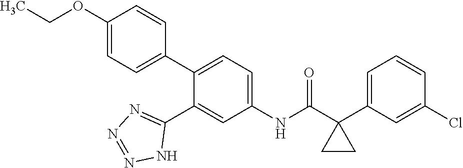 [SMILES: CCOc1ccc(-c2ccc(NC(=O)C3(c4cccc(Cl)c4)CC3)cc2-c2nnn[nH]2)cc1] | 1-(3- chlorophenyl)- N-[4′- (methoxymethyl)- 2-(1H- tetrazol-5- yl(biphenyl-4- yl]cyclopropane carboxamide | ¹H NMR (500 MHz, DMSO-d6) δ [ppm] 9.49 (s, 1H), 7.95-7.81 (m, 2H), 7.47 (d, J = 8.8 Hz, 1H), 7.44 (s, 1H), 7.41-7.33 (m, 3H), 7.22 (d, J = 7.9 Hz, 2H), 7.01 (d, J = 7.9 Hz, 2H), 4.37 (s, 2H), 3.27 (s, 3H), 1.49 (m, 2H), 1.19 (m, 2H). LCMS (Analytical Method F): Rt = 3.57 mins; MS (ESIpos) m/z = 460.1 (M + H)⁺. |
| 124 | 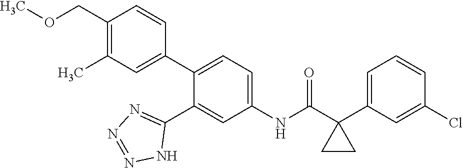 [SMILES: COCc1ccc(-c2ccc(NC(=O)C3(c4cccc(Cl)c4)CC3)cc2-c2nnn[nH]2)cc1C] | 1-(3- chlorophenyl)- N-[4′- (methoxymethyl)- 3′-methyl-2- (1H-tetrazol-5- yl)biphenyl-4- yl]cyclopropane carboxamide | ¹H NMR (500 MHz, DMSO-d6) δ [ppm] 1.11-1.24 (m, 2H), 1.43- 1.54 (m, 2H), 2.19 (s, 3H), 3.30 (s, 3H), 4.37 (s, 2H), 6.68-6.87 (m, 1H), 6.88-7.00 (m, 1H), 7.15 (d, J = 7.9 Hz, 1H), 7.29- 7.55 (m, 5H), 7.81-7.93 (m, 2H), 9.48 (s, 1H). LCMS (Analytical Method F): Rt = 3.75 mins; MS (ESIpos) m/z = 474.2 (M + H)⁺. |
| 125 | 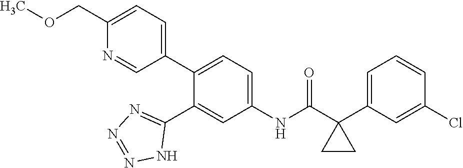 [SMILES: COCc1ccc(-c2ccc(NC(=O)C3(c4cccc(Cl)c4)CC3)cc2-c2nnn[nH]2)cn1] | 1-(3- chlorophenyl)- N-{4-[6- (methoxymethyl) pyridin-3-yl]-3- (1H-tetrazol-5- yl)phenyl} cyclopropane- carboxamide | ¹H NMR (500 MHz, DMSO-d6) δ [ppm] 1.09-1.29 (m, 2H), 1.40- 1.57 (m, 2H), 3.36 (s, 3H), 4.48 (s, 2H), 7.33 (d, J = 8.1 Hz, 1H), 7.34-7.42 (m, 3H), 7.43-7.48 (m, 2H), 7.52 (d, J = 8.5 Hz, 1H), 7.91 (dd, J = 8.5, 2.2 Hz, 1H), 7.99 (d, J = 1.7 Hz, 1H), 8.22 (d, J = 2.1 Hz, 1H), 9.55 (s, 1H). LCMS (Analytical Method F): Rt = 2.86 mins; MS (ESIpos): m/z = 461.0 (M + H)⁺. |
| 126 | 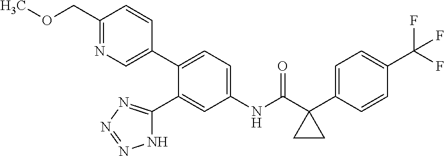 [SMILES: COCc1ccc(-c2ccc(NC(=O)C3(c4ccc(C(F)(F)F)cc4)CC3)cc2-c2nnn[nH]2)cn1] | N-{4-[6- (methoxymethyl) pyridin-3-yl]-3- (1H-tetrazol-5- yl)phenyl}-1-[4- (trifluoromethyl) phenyl] cyclopropane- carboxamide | ¹H NMR (500 MHz, DMSO-d6) δ [ppm] 1.21-1.31 (m, 2H), 1.52- 1.61 (m, 2H), 3.36 (s, 3H), 4.48 (s, 2H), 7.33 (d, J = 8.1 Hz, 1H), 7.45 (dd, J = 8.1, 2.2 Hz, 1H), 7.53 (d, J = 8.5 Hz, 1H), 7.61 (d, J = 8.1 Hz, 2H), 7.72 (d, J = 8.2 Hz, 2H), 7.92 (dd, J = 8.5, 2.2 Hz, 1H), 7.97-8.05 (m, 1H), 8.22 (d, J = 1.8 Hz, 1H), 9.65 (s, 1H). LCMS (Analytical Method F): Rt = 3.04 mins; MS (ESIpos): m/z = 495.1 (M + H)⁺. |
| 127 | 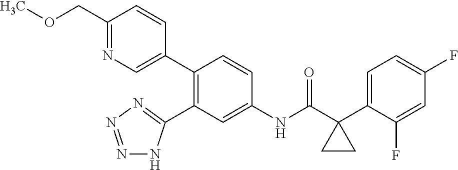 [SMILES: COCc1ccc(-c2ccc(NC(=O)C3(c4ccc(F)cc4F)CC3)cc2-c2nnn[nH]2)cn1] | 1-(2,4- difluorophenyl)- N-{4-[6- (methoxymethyl) pyridin-3-yl]-3- (1H-tetrazol-5- yl)phenyl} cyclopropane- carboxamide | ¹H NMR (250 MHz, DMSO-d6) δ [ppm] 9.20 (s, 1H), 8.40 (d, J = 1.9 Hz, 1H), 8.01 (d, J = 2.0 Hz, 1H), 7.93 (dd, J = 8.5, 2.2 Hz, 1H), 7.75-7.63 (m, 1H), 7.61- 7.46 (m, 3H), 7.33-7.19 (m, 1H), 7.18-7.05 (m, 1H), 4.59 (s, 2H), 3.40 (s, 3H), 1.69-1.54 (m, 2H), 1.23-1.11 (m, 2H). LCMS (Analytical Method F): Rt = 0.97 mins; MS (ESIpos) m/z = 463 (M + H)⁺. |
| 128 | 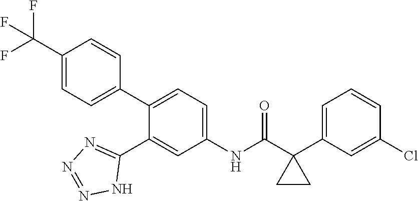 [SMILES: O=C(Nc1ccc(-c2ccc(C(F)(F)F)cc2)c(-c2nnn[nH]2)c1)C1(c2cccc(Cl)c2)CC1] | 1-(3- chlorophenyl)- N-[2-(1H- tetrazol-5-yl)- 4′- (trifluoromethyl) biphenyl-4- yl]cyclopropane carboxamide | ¹H NMR (250 MHz, DMSO-d6) δ [ppm] 1.12-1.28 (m, 2H), 1.40- 1.60 (m, 2H), 7.28 (d, J = 8.0 Hz, 2H), 7.33-7.43 (m, 3H), 7.43- 7.48 (m, 1H), 7.54 (d, J = 8.5 Hz, 1H), 7.67 (d, J = 8.2 Hz, 2H), 7.93 (dd, J = 8.5, 2.2 Hz, 1H), 8.00 (d, J = 2.0 Hz, 1H), 9.57 (s, 1H). LCMS (Analytical Method F): Rt = 3.94 mins; MS (ESIpos): m/z = 484.0 (M + H)⁺. |
| 129 | 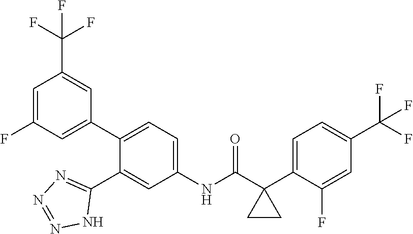 [SMILES: O=C(Nc1ccc(-c2cc(F)cc(C(F)(F)F)c2)c(-c2nnn[nH]2)c1)C1(c2ccc(C(F)(F)F)cc2F)CC1] | N-[3′-fluoro-2- (1H-tetrazol-5- yl)-5′- (trifluoromethyl) biphenyl-4-yl]- 1-[2-fluoro-4- (trifluoromethyl) phenyl] cyclopropane- carboxamide | ¹H NMR (400 MHz, DMSO-d6) δ [ppm] 1.25-1.28 (m, 2H), 1.50- 1.52 (m, 2H), 7.17 (s, 1H), 7.29- 7.33 (m, 1H), 7.55 (d, 1H), 7.61- 7.63 (m, 2H), 7.69-7.74 (m, 2H), 7.89 (dd, 1H), 7.95 (d, 1H), 9.31 (s, 1H). LCMS (method 4): Rt = 0.85 min, m/z = 554 (M + H)⁺. |
| 130 | 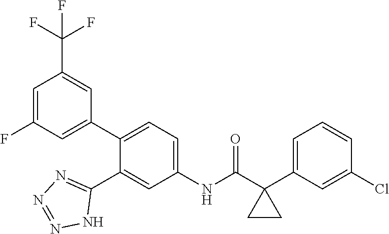 [SMILES: O=C(Nc1ccc(-c2cc(F)cc(C(F)(F)F)c2)c(-c2nnn[nH]2)c1)C1(c2cccc(Cl)c2)CC1] | 1-(3- chlorophenyl)- N-[3′-fluoro-2- (1H-tetrazol-5- yl)-5′- (trifluoromethyl) biphenyl-4- yl]cyclopropane carboxamide | ¹H NMR (400 MHz, DMSO-d6) δ [ppm] 1.19-1.22 (m, 2H), 1.50- 1.52 (m, 2H), 7.17 (s, 1H), 7.27- 7.31 (m, 1H), 7.35-7.42 (m, 3H), 7.45-7.46 (m, 1H), 7.54 (d, 1H), 7.58-7.62 (m, 1H), 7.90 (dd, 1H), 8.00 (d, 1H), 9.57 (s, 1H). LCMS (method 4): Rt = 0.82 min, m/z = 502 (M + H)⁺. |
| 131 | 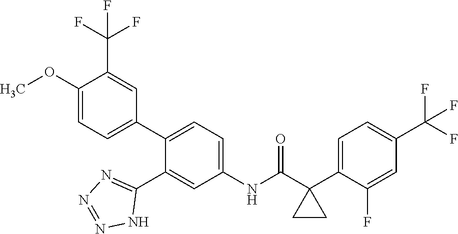 [SMILES: COc1ccc(-c2ccc(NC(=O)C3(c4ccc(C(F)(F)F)cc4F)CC3)cc2-c2nnn[nH]2)cc1C(F)(F)F] | 1-[2-fluoro-4- (trifluoromethyl) phenyl]-N-[4′- methoxy-2-(1H- tetrazol-5-yl)- 3′- (trifluoromethyl) biphenyl-4- yl]cyclopropane carboxamide | ¹H NMR (400 MHz, DMSO-d6) δ [ppm] 1.25-1.27 (m, 2H), 1.64- 1.67 (m, 2H), 3.88 (s, 3H), 7.17- 7.20 (m, 1H), 7.24-7.27 (m, 2H), 7.52 (d, 1H), 7.60-7.74 (m, 3H), 7.86-7.89 (m, 2H), 9.27 (s, 1H). LCMS (method 3): Rt = 1.35 min, m/z = 566 (M + H)⁺. |
| 132 | 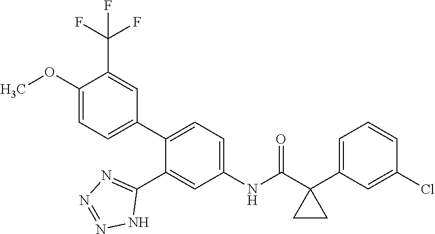 [SMILES: COc1ccc(-c2ccc(NC(=O)C3(c4cccc(Cl)c4)CC3)cc2-c2nnn[nH]2)cc1C(F)(F)F] | 1-(3- chlorophenyl)- N-[4′-methoxy- 2-(1H-tetrazol- 5-yl)-3′- (trifluoromethyl) biphenyl-4- yl]cyclopropane carboxamide | ¹H NMR (400 MHz, DMSO-d6) δ [ppm] 1.19-1.22 (m, 2H), 1.49- 1.52 (m, 2H), 3.88 (s, 3H), 7.17- 7.20 (m, 1H), 7.24-7.27 (m, 2H), 7.35-7.41 (m, 3H), 7.45 (m, 1H), 7.52 (d, 1H), 7.90 (dd, 1H), 7.94 (d, 1H), 9.54 (s, 1H). LCMS (method 3): Rt = 1.33 min, m/z = 514 (M + H)⁺. |
| 133 | 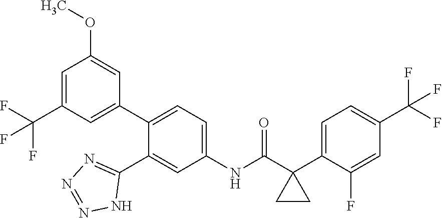 [SMILES: COc1cc(-c2ccc(NC(=O)C3(c4ccc(C(F)(F)F)cc4F)CC3)cc2-c2nnn[nH]2)cc(C(F)(F)F)c1] | 1-[2-fluoro-4- (trifluoromethyl) phenyl]-N-[3′- methoxy-2-(1H- tetrazol-5-yl)- 5′- (trifluoromethyl) biphenyl-4- yl]cyclopropane carboxamide | ¹H NMR (400 MHz, DMSO-d6) δ [ppm] 1.25-1.28 (m, 2H), 1.65- 1.68 (m, 2H), 3.75 (s, 3H), 6.88 (s, 1H), 6.90 (s, 1H), 7.58 (d, 1H), 7.58-7.74 (m, 3H), 7.88- 7.91 (m, 2H), 9.30 (s, 1H). LCMS (method 3): Rt = 1.38 min, m/z = 566 (M + H)⁺. |
| 134 | 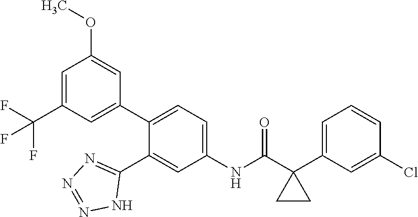 [SMILES: COc1cc(-c2ccc(NC(=O)C3(c4cccc(Cl)c4)CC3)cc2-c2nnn[nH]2)cc(C(F)(F)F)c1] | 1-(3- chlorophenyl)- N-[3′-methoxy- 2-(1H-tetrazol- 5-yl)-5′- (trifluoromethyl) biphenyl-4- yl]cyclopropane carboxamide | ¹H NMR (400 MHz, DMSO-d6) δ [ppm] 1.20-1.22 (m, 2H), 1.50- 1.52 (m, 2H), 3.75 (s, 3H), 6.87 (s, 1H), 6.90 (s, 1H), 7.17 (s, 1H), 7.36-7.41 (m, 3H), 7.45 (m, 1H), 7.59 (d, 1H), 7.92 (dd, 1H), 7.97 (d, 1H), 9.58 (s, 1H). LCMS (method 3): Rt = 1.36 min, m/z = 514 (M + H)⁺. |
| 135 | 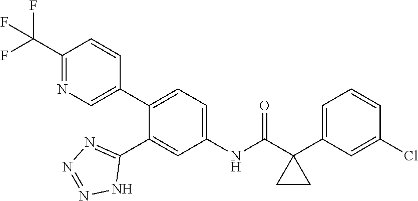 [SMILES: O=C(Nc1ccc(-c2ccc(C(F)(F)F)nc2)c(-c2nnn[nH]2)c1)C1(c2cccc(Cl)c2)CC1] | 1-(3- chlorophenyl)- N-{3-(1H- tetrazol-5-yl)-4- [6- (trifluoromethyl) pyridin-3- yl]phenyl} cyclopropane- carboxamide | ¹H NMR (500 MHz, DMSO-d6) δ [ppm] 9.62 (s, 1H), 8.52 (d, J = 1.9 Hz, 1H), 8.13-8.08 (m, 1H), 7.95 (dd, J = 8.5, 2.2 Hz, 1H), 7.85 (d, J = 8.1 Hz, 1H), 7.76 (d, J = 1.6 Hz, 1H), 7.59 (d, J = 8.5 Hz, 1H), 7.45 (d, J = 1.7 Hz, 1H), 7.43-7.34 (m, 3H), 1.55-1.48 (m, 2H), 1.25-1.19 (m, 2H). LCMS (Analytical Method D): Rt = 4.45 mins; MS (ESIpos) m/z = 485.15 (M + H)⁺. |
| 136 | 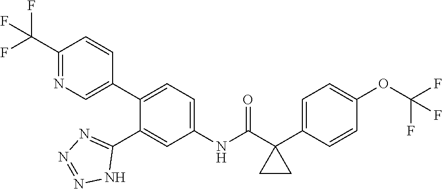 [SMILES: O=C(Nc1ccc(-c2ccc(C(F)(F)F)nc2)c(-c2nnn[nH]2)c1)C1(c2ccc(OC(F)(F)F)cc2)CC1] | N-{3-(1H- tetrazol-5-yl)-4- [6- (trifluoromethyl) pyridin-3- yl]phenyl}-1-[4- (trifluoromethoxy) phenyl] cyclopropane- carboxamide | LCMS (method 1): Rt = 1.33 min, m/z = 535 (M + H)⁺ |
| 137 | 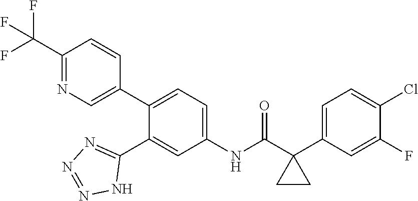 [SMILES: O=C(Nc1ccc(-c2ccc(C(F)(F)F)nc2)c(-c2nnn[nH]2)c1)C1(c2ccc(Cl)c(F)c2)CC1] | 1-(4-Chloro-3- fluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.21-1.23 (m, 2H), 1.52- 1.54 (m, 2H), 7.30 (dd, 1H), 7.47 (dd, 1H), 7.51 (d, 1H), 7.58 (dd, 1H), 7.75 (dd, 1H), 7.83 (d, 1H), 7.90 (dd, 1H), 8.05 (d, 1H), 8.49 (d, 1H), 9.47 (s, 1H). LCMS (method 4): Rt = 0.79 min, m/z = 503 (M + H)+ |
| 138 | 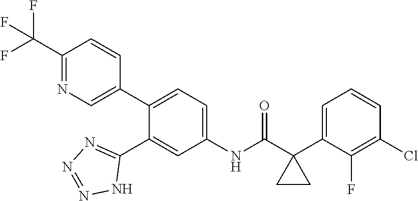 [SMILES: O=C(Nc1ccc(-c2ccc(C(F)(F)F)nc2)c(-c2nnn[nH]2)c1)C1(c2cccc(Cl)c2F)CC1] | 1-(3-chloro-2- fluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.22-1.25 (m, 2H), 1.64- 1.66 (m, 2H), 7.24-7.28 (m, 1H), 7.47 (dt, 1H), 7.52 (d, 1H), 7.58 (dt, 1H), 7.76 (dd, 1H), 7.83 (d, 1H), 7.89 (dd, 1H), 8.02 (d, 1H), 8.50 (d, 1H), 9.28 (s, 1H). LCMS (method 4): Rt = 0.77 min, m/z = 503 (M + H)⁺ |
| 139 | 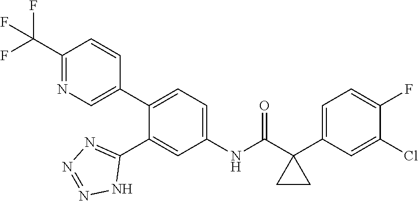 [SMILES: O=C(Nc1ccc(-c2ccc(C(F)(F)F)nc2)c(-c2nnn[nH]2)c1)C1(c2ccc(F)c(Cl)c2)CC1] | 1-(3-chloro-4- fluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.18-1.21 (m, 2H), 1.50- 1.53 (m, 2H), 7.33-7.35 (m, 1H), 7.41-7.50 (m, 2H), 7.64 (dd, 1H), 7.73-7.76 (m, 1H), 7.82 (d, 1H), 7.87-7.90 (m, 1H), 8.02 (d, 1H), 8.48 (m, 1H), 9.39 (s, 1H). LCMS (method 3): Rt = 1.27 min, m/z = 503 (M + H)⁺. |
| 140 | 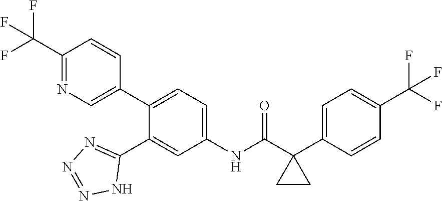 [SMILES: O=C(Nc1ccc(-c2ccc(C(F)(F)F)nc2)c(-c2nnn[nH]2)c1)C1(c2ccc(C(F)(F)F)cc2)CC1] | N-{3-(1H- tetrazol-5-yl)-4- [6- (trifluoromethyl) pyridin-3- yl]phenyl}-1-[4- (trifluoromethyl) phenyl] cyclopropane- carboxamide | ¹H NMR (400 MHz, DMSO-d6) δ [ppm] 1.24-1.27 (m, 2H), 1.56- 1.59 (m, 2H), 7.56 (d, 1H), 7.61 (d, 2H), 7.72-7.77 (m, 3H), 7.84 (d, 1H), 7.94 (dd, 1H), 8.10 (d, 1H), 8.10 (d, 1H), 8.51 (d, 1H), 9.72 (s, 1H). LCMS (method 4): Rt = 0.80 min, m/z = 519 (M + H)⁺. |
| 141 | 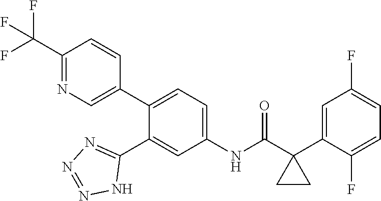 [SMILES: O=C(Nc1ccc(-c2ccc(C(F)(F)F)nc2)c(-c2nnn[nH]2)c1)C1(c2cc(F)ccc2F)CC1] | 1-(2,5- difluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.22-1.25 (m, 2H), 1.59- 1.62 (m, 2H), 7.23-7.31 (m, 2H), 7.34-7.39 (m, 1H), 7.54 (d, 1H), 7.76 (dd, 1H), 7.84 (d, 1H), 7.91 (dd, 1H), 8.03 (d, 1H), 8.51 (d, 1H), 9.30 (s, 1H). LCMS (method 4): Rt = 0.72 min, m/z = 487 (M + H)⁺. |
| 142 | 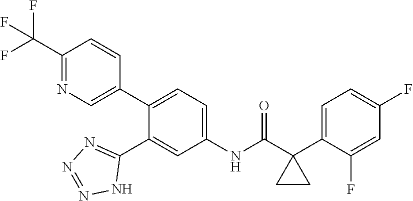 [SMILES: O=C(Nc1ccc(-c2ccc(C(F)(F)F)nc2)c(-c2nnn[nH]2)c1)C1(c2ccc(F)cc2F)CC1] | 1-(2,4- difluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.17-1.20 (m, 2H), 1.60- 1.63 (m, 2H), 7.12 (tdd, 1H), 7.27 (ddd, 1H), 7.51-7.57 (m, 2H), 7.76 (dd, 1H), 7.84 (d, 1H), 7.92 (dd, 1H), 8.03 (d, 1H), 8.51 (d, 1H), 9.21 (s, 1H). LCMS (method 4): Rt = 0.74 min, m/z = 487 (M + H)⁺. |
| 143 | 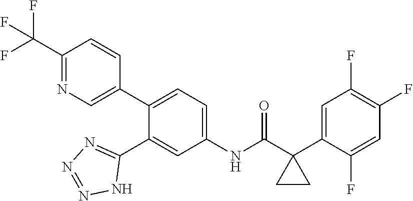 [SMILES: O=C(Nc1ccc(-c2ccc(C(F)(F)F)nc2)c(-c2nnn[nH]2)c1)C1(c2cc(F)c(F)cc2F)CC1] | N-{3-(1H- tetrazol-5-yl)-4- [6- (trifluoromethyl) pyridin-3- yl]phenyl}-1- (2,4,5- trifluorophenyl) cyclopropane- carboxamide | ¹H NMR (400 MHz, DMSO-d6) δ [ppm] 1.22-1.25 (m, 2H), 1.58- 1.61 (m, 2H), 7.51-7.70 (m, 3H), 7.76 (dd, 1H), 7.84 (d, 1H), 7.91 (dd, 1H), 8.02 (d, 1H), 8.51 (d, 1H), 9.24 (s, 1H). LCMS (method 1): Rt = 1.21 min, m/z = 505 (M + H)⁺. |
| 144 | 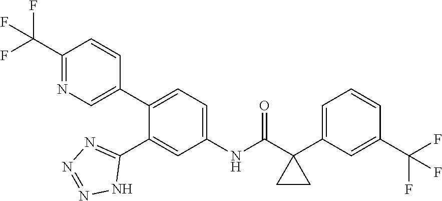 [SMILES: O=C(Nc1ccc(-c2ccc(C(F)(F)F)nc2)c(-c2nnn[nH]2)c1)C1(c2cccc(C(F)(F)F)c2)CC1] | N-{3-(1H- tetrazol-5-yl)-4- [6- (trifluoromethyl) pyridin-3- yl]phenyl}-1-[3- (trifluoromethyl) phenyl] cyclopropane- carboxamide | ¹H NMR (400 MHz, DMSO-d6) δ [ppm] 1.24-1.28 (m, 2H), 1.56- 1.58 (m, 2H), 7.56 (d, 1H), 7.63 (d, 1H), 7.68 (d, 1H), 7.71-7.76 (m, 3H), 7.84 (d, 1H), 7.92 (dd, 1H), 8.08 (d, 1H), 8.51 (d, 1H), 9.68 (s, 1H). LCMS (method 4): Rt = 0.79 min, m/z = 519 (M + H)⁺. |
| 145 | 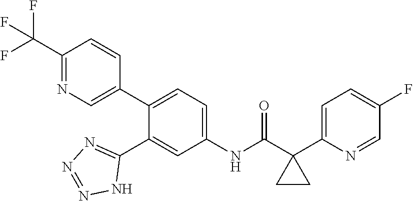 [SMILES: O=C(Nc1ccc(-c2ccc(C(F)(F)F)nc2)c(-c2nnn[nH]2)c1)C1(c2ccc(F)cn2)CC1] | 1-(5- fluoropyridin-2- yl)-N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.37-1.40 (m, 2H), 1.53- 1.56 (m, 2H), 7.47 (dd, 1H), 7.60 (d, 1H), 7.72 (dt, 1H), 7.76 (dd, 1H), 7.86 (d, 1H), 7.97 (dd, 1H), 8.17 (d, 1H), 8.53 (d, 1H), 8.56 (d, 1H), 10.32 (s, 1H). LCMS (method 4): Rt = 0.67 min, m/z = 470 (M + H)⁺. |
| 146 | 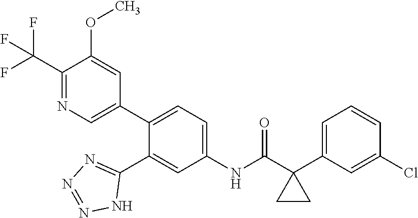 [SMILES: COc1cc(-c2ccc(NC(=O)C3(c4cccc(Cl)c4)CC3)cc2-c2nnn[nH]2)cnc1C(F)(F)F] | 1-(3- chlorophenyl)- N-{4-[5- methoxy-6- (trifluoromethyl) pyridin-3-yl]-3- (1H-tetrazol-5- yl)phenyl} cyclopropane- carboxamide | ¹H NMR (400 MHz, DMSO-d6) δ [ppm] 1.19-1.22 (m, 2H), 1.49- 1.52 (m, 2H), 3.80 (s, 3H), 7.35- 7.40 (m, 3H), 7.45-7.47 (m, 2H), 7.56 (d, 1H), 7.89-7.91 (m, 2H), 8.02 (d, 1H), 8.48 (m, 1H), 9.58 (s, 1H). LCMS (method 4): Rt = 0.76 min, m/z = 515 (M + H)⁺. |
| 147 | 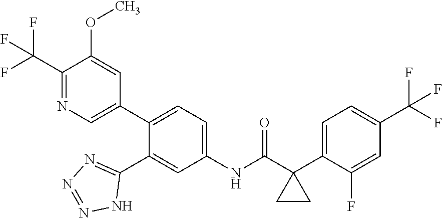 | 1-[2-fluoro-4- (trifluoromethyl) phenyl]-N-{4- [5-methoxy-6- (trifluoromethyl) pyridin-3-yl]-3- (1H-tetrazol-5- yl)phenyl} cyclopropane- carboxamide | ¹H NMR (400 MHz, DMSO-d6) δ [ppm] 1.26-1.28 (m, 2H), 1.65- 1.68 (m, 2H), 3.82 (s, 3H), 7.50 (s, 1H), 7.60-7.63 (m, 2H), 7.69- 7.74 (m, 2H), 7.89 (m, 1H), 7.92 (dd, 1H), 8.01 (d, 1H), 9.34 (s, 1H). LCMS (method 4): Rt = 0.80 min, m/z = 5675 (M + H)⁺. |
| 148 | 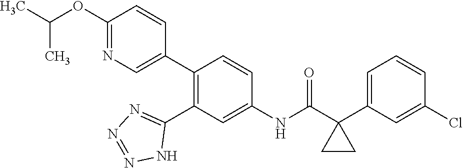 [SMILES: CC(C)Oc1ccc(-c2ccc(NC(=O)C3(c4cccc(Cl)c4)CC3)cc2-c2nnn[nH]2)cn1] | 1-(3- chlorophenyl)- N-[4-(6- isopropoxy- pyridin-3-yl)-3- (1H-tetrazol-5- yl)phenyl] cyclopropane- carboxamide | ¹H NMR (500 MHz, DMSO-d6) δ [ppm] 1.15-1.24 (m, 2H), 1.27 (d, J = 6.2 Hz, 6H), 1.43-1.53 (m, 2H), 5.21 (hept, J = 6.2 Hz, 1H), 6.64 (d, J = 8.6 Hz, 1H), 7.24-7.30 (m, 1H), 7.33-7.42 (m, 3H), 7.43-7.46 (m, 1H), 7.49 (d, J = 8.3 Hz, 1H), 7.86- 7.91 (m, 2H), 7.94 (s, 1H), 9.52 (s, 1H). LCMS (Analytical Method F): Rt = 3.80 mins; MS (ESIpos) m/z = 475.1 (M + H)⁺. |
| 149 | 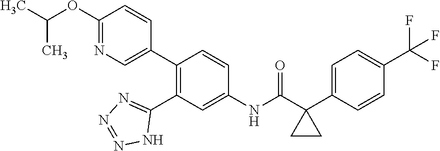 [SMILES: CC(C)Oc1ccc(-c2ccc(NC(=O)C3(c4ccc(C(F)(F)F)cc4)CC3)cc2-c2nnn[nH]2)cn1] | N-[4-(6- isopropoxy- pyridin-3-yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 1.21-1.26 (m, 2H), 1.27 (d, J = 6.2 Hz, 6H), 1.51-1.59 (m, 2H), 5.21 (hept, J = 6.3 Hz, 1H), 6.64 (d, J = 8.5 Hz, 1H), 7.27 (dd, J = 8.5, 2.4 Hz, 1H), 7.49 (d, J = 8.4 Hz, 1H), 7.60 (d, J = 8.1 Hz, 2H), 7.72 (d, J = 8.2 Hz, 2H), 7.89 (dt, J = 5.8, 2.4 Hz, 2H), 7.95 (s, 1H), 9.62 (s, 1H). LCMS (Analytical Method F): Rt = 3.91 mins; MS (ESIpos) m/z = 509.1 (M + H)⁺. |
| 150 | 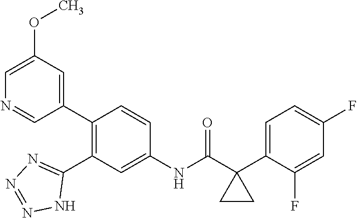 [SMILES: COc1cncc(-c2ccc(NC(=O)C3(c4ccc(F)cc4F)CC3)cc2-c2nnn[nH]2)c1] | 1-(2,4- difluorophenyl)- N-[4-(5- methoxypyridin- 3-yl)-3-(1H- tetrazol-5- yl)phenyl] cyclopropane- carboxamide | ¹H NMR (500 MHz, Methanol-d4) δ [ppm] 8.88 (s, 1H), 8.17 (s, 1H), 7.88 (m, 2H), 7.79 (m, 1H), 7.54 (m, 2H), 7.19-7.11 (m, 1H), 7.07-6.96 (m, 2H), 3.79 (s, 3H), 1.76-1.65 (m, 2H), 1.26-1.14 (m, 2H). LCMS (Analytical Method F): Rt = 2.59 mins; MS (ESIpos) m/z = 449 (M + H)⁺. |
| 151 | 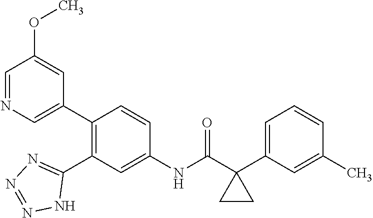 [SMILES: COc1cncc(-c2ccc(NC(=O)C3(c4cccc(C)c4)CC3)cc2-c2nnn[nH]2)c1] | N-[4(5- methoxypyridin- 3-yl)-3-(1H- tetrazol-5- yl)phenyl]-1-(3- methylphenyl) cyclopropane- carboxamide | ¹H NMR (500 MHz, DMSO-d6) δ [ppm] 9.26 (s, 1H), 8.13 (s, 1H), 7.93-7.75 (m, 3H), 7.43 (d, J = 8.4 Hz, 1H), 7.32-7.15 (m, 3H), 7.11 (d, J = 7.4 Hz, 1H), 7.02 (s, 1H), 3.71 (s, 3H), 2.32 (s, 3H), 1.45 (m, 2H), 1.17-1.02 (m, 2H). LCMS (Analytical Method F): Rt = 3.54 mins; MS (ESIpos) m/z = 427 (M + H)⁺. |
| 152 | 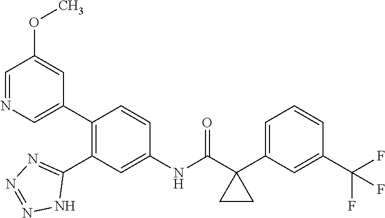 [SMILES: COc1cncc(-c2ccc(NC(=O)C3(c4cccc(C(F)(F)F)c4)CC3)cc2-c2nnn[nH]2)c1] | N-[4-(5- methoxypyridin- 3-yl)-3-(1H- tetrazol-5- yl)phenyl]-1-[3- (trifluoromethyl) phenyl] cyclopropane- carboxamide | ¹H NMR (500 MHz, DMSO-d6) δ [ppm] 9.53 (s, 1H), 8.13 (d, J = 2.6 Hz, 1H), 7.95-7.77 (m, 3H), 7.77-7.67 (m, 2H), 7.67-7.54 (m, 2H), 7.43 (d, J = 8.4 Hz, 1H), 7.09-6.97 (m, 1H), 3.70 (s, 3H), 1.61-1.47 (m, 2H), 1.30-1.15 (m, 2H). LCMS (Analytical Method F): Rt = 3.70 mins; MS (ESIpos) m/z = 481 (M + H)⁺. |
| 153 | 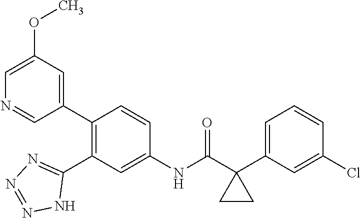 [SMILES: COc1cncc(-c2ccc(NC(=O)C3(c4cccc(Cl)c4)CC3)cc2-c2nnn[nH]2)c1] | 1-(3- chlorophenyl)- N-[4-(5- methoxypyridin- 3-yl)-3-(1H- tetrazol-5- yl)phenyl] cyclopropane- carboxamide | ¹H NMR (500 MHz, DMSO-d6) δ [ppm] 9.54 (s, 1H), 8.18 (d, J = 2.8 Hz, 1H), 7.96 (d, J = 2.2 Hz, 1H), 7.90 (dd, J = 8.5, 2.2 Hz, 1H), 7.84 (d, J = 1.7 Hz, 1H), 7.52 (d, J = 8.5 Hz, 1H), 7.45 (d, J = 1.7 Hz, 1H), 7.43-7.33 (m, 3H), 7.08-7.01 (m, 1H), 6.52 (s, 1H), 3.74 (s, 3H), 1.57-1.45 (m, 2H), 1.28-1.13 (m, 2H) LCMS (Analytical Method F): Rt = 2.81 mins; MS (ESIpos) m/z = 447 (M + H)⁺. |
| 154 | 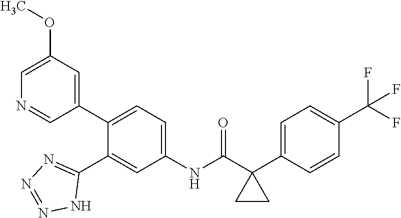 [SMILES: COc1cncc(-c2ccc(NC(=O)C3(c4ccc(C(F)(F)F)cc4)CC3)cc2-c2nnn[nH]2)c1] | N-[4-(5- methoxypyridin- 3-yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.65 (s, 1H), 8.20 (d, J = 2.7 Hz, 1H), 7.99 (d, J = 2.0 Hz, 1H), 7.92 (dd, J = 8.5, 2.2 Hz, 1H), 7.82 (d, J = 1.6 Hz, 1H), 7.72 (d, J = 8.3 Hz, 2H), 7.61 (d, J = 8.2 Hz, 2H), 7.56 (d, J = 8.5 Hz, 1H), 7.08-7.01 (m, 1H), 3.74 (s, 3H), 1.61-1.52 (m, 2H), 1.27-1.22 (m, 2H). LCMS (Analytical Method F): Rt = 2.99 mins; MS (ESIpos) m/z = 481.1 (M + H)⁺. |
| 155 | 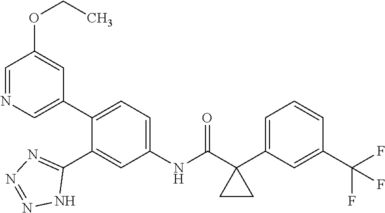 [SMILES: CCOc1cncc(-c2ccc(NC(=O)C3(c4cccc(C(F)(F)F)c4)CC3)cc2-c2nnn[nH]2)c1] | N-[4-(5- ethoxypyridin-3- yl)-3-(1H- tetrazol-5- yl)phenyl]-1-[3- (trifluoromethyl) phenyl] cyclopropane- carboxamide, as a formic acid salt | ¹H NMR (500 MHz, DMSO-d6) δ [ppm] 9.57 (s, 1H), 8.15-8.13 (m, 1H), 8.13 (s, 1H), 7.94-7.90 (m, 1H), 7.87-7.84 (m, 1H), 7.84 (d, J = 1.7 Hz, 1H), 7.72 (d, J = 7.7 Hz, 1H), 7.71 (s, 1H), 7.66 (d, J = 7.8 Hz, 1H), 7.63- 7.58 (m, 1H), 7.47 (d, J = 8.4 Hz, 1H), 7.01-6.98 (m, 1H), 3.97 (q, J = 7.0 Hz, 2H), 1.60-1.51 (m, 2H), 1.28 (t, J = 7.0 Hz, 3H), 1.26-1.22 (m, 2H). LCMS (Analytical Method F): Rt = 3.10 mins; MS (ESIpos) m/z = 495 (M + H)⁺. |
| 156 | 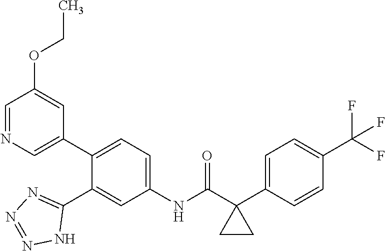 [SMILES: CCOc1cncc(-c2ccc(NC(=O)C3(c4ccc(C(F)(F)F)cc4)CC3)cc2-c2nnn[nH]2)c1] | N-[4-(5- ethoxypyridin-3- yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.65 (s, 1H), 8.18 (s, 1H), 7.99 (s, 1H), 7.92 (dd, J = 8.5, 2.2 Hz, 1H), 7.83 (s, 1H), 7.72 (d, J = 8.2 Hz, 2H), 7.61 (d, J = 8.1 Hz, 2H), 7.56 (d, J = 8.5 Hz, 1H), 7.02 (s, 1H), 4.00 (q, J = 7.0 Hz, 2H), 1.59-1.54 (m, 2H), 1.29 (t, J = 7.0 Hz, 3H), 1.26- 1.23 (m, 2H). LCMS (Analytical Method F): Rt = 3.15 mins; MS (ESIpos) m/z = 495 (M + H)⁺. |
| 157 | 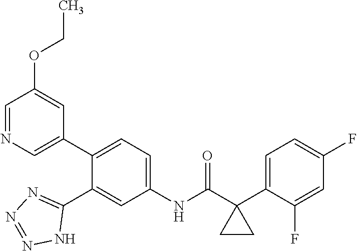 [SMILES: CCOc1cncc(-c2ccc(NC(=O)C3(c4ccc(F)cc4F)CC3)cc2-c2nnn[nH]2)c1] | 1-(2,4- difluorophenyl)- N-[4-(5- ethoxypyridin-3- yl)-3-(1H- tetrazol-5- yl)phenyl] cyclopropane- carboxamide | ¹H NMR (500 MHz, DMSO-d6) δ [ppm] 9.15 (s, 1H), 8.18 (s, 1H), 7.91 (d, J = 8.6 Hz, 2H), 7.82 (s, 1H), 7.57-7.49 (m, 2H), 7.25 (td, J = 10.4, 2.5 Hz, 1H), 7.10 (td, J = 8.5, 2.4 Hz, 1H), 7.02 (s, 1H), 3.99 (q, J = 6.9 Hz, 2H), 1.64-1.57 (m, 2H), 1.28 (t, J = 6.9 Hz, 3H), 1.21-1.11 (m, 2H). LCMS (Analytical Method F): Rt = 2.77 mins; MS (ESIpos) m/z = 463 (M + H)⁺. |
| 158 | 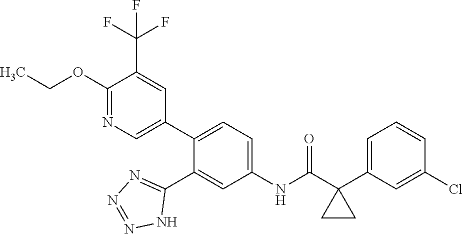 [SMILES: CCOc1ncc(-c2ccc(NC(=O)C3(c4cccc(Cl)c4)CC3)cc2-c2nnn[nH]2)cc1C(F)(F)F] | 1-(3- chlorophenyl)- N-{4-[6-ethoxy- 5- (trifluoromethyl) pyridin-3-yl]-3- (1H-tetrazol-5- yl)phenyl} cyclopropane- carboxamide | ¹H NMR (500 MHz, DMSO-d6) δ [ppm] 9.56 (s, 1H), 8.12 (d, J = 2.2 Hz, 1H), 8.08-8.01 (m, 1H), 7.90 (dd, J = 8.5, 2.2 Hz, 1H), 7.71 (d, J = 2.2 Hz, 1H), 7.56 (d, J = 8.5 Hz, 1H), 7.47-7.43 (m, 1H), 7.42-7.33 (m, 3H), 4.45 (q, J = 7.0 Hz, 2H), 1.54-1.48 (m, 2H), 1.33 (t, J = 7.0 Hz, 3H), 1.23-1.18 (m, 2H). LCMS (Analytical Method F): Rt = 4.14 mins; MS (ESIpos) m/z = 529.0 (M + H)⁺. |
| 159 | 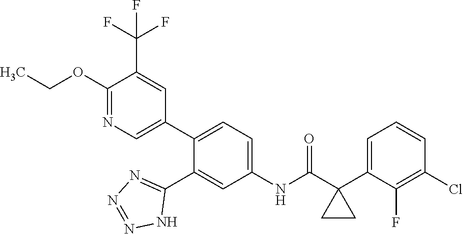 [SMILES: CCOc1ncc(-c2ccc(NC(=O)C3(c4cccc(Cl)c4F)CC3)cc2-c2nnn[nH]2)cc1C(F)(F)F] | 1-(3-chloro-2- fluorophenyl)- N-{4-[6-ethoxy- 5- (trifluoromethyl) pyridin-3-yl]-3- (1H-tetrazol-5- yl)phenyl} cyclopropane- carboxamide | ¹H NMR (500 MHz, DMSO-d6) δ [ppm] 9.25 (s, 1H), 8.13 (d, J = 2.1 Hz, 1H), 7.99 (s, 1H), 7.88 (dd, J = 8.5, 2.2 Hz, 1H), 7.72 (d, J = 2.2 Hz, 1H), 7.62-7.52 (m, 2H), 7.50-7.41 (m, 1H), 7.30- 7.20 (m, 1H), 4.45 (q, J = 7.0 Hz, 2H), 1.70-1.57 (m, 2H), 1.33 (t, J = 7.0 Hz, 3H), 1.29-1.17 (m, 2H). LCMS (Analytical Method F): Rt = 4.08 mins; MS (ESIpos) m/z = 547.1 (M + H)⁺. |
| 160 | 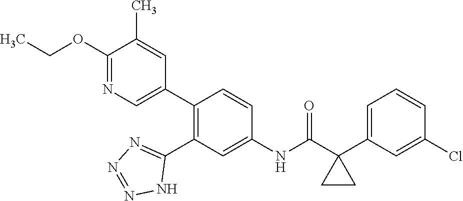 [SMILES: CCOc1ncc(-c2ccc(NC(=O)C3(c4cccc(Cl)c4)CC3)cc2-c2nnn[nH]2)cc1C] | 1-(3- chlorophenyl)- N-[4-(6-ethoxy- 5- methylpyridin- 3-yl)-3-(1H- tetrazol-5- yl)phenyl] cyclopropane- carboxamide | ¹H NMR (500 MHz, DMSO-d6) δ [ppm] 9.50 (s, 1H), 7.93 (s, 1H), 7.88 (dd, J = 8.5, 2.2 Hz, 1H), 7.63 (d, J = 2.3 Hz, 1H), 7.47 (d, J = 8.5 Hz, 1H), 7.44 (d, J = 1.7 Hz, 1H), 7.42-7.34 (m, 3H), 7.22 (d, J = 1.6 Hz, 1H), 4.30 (q, J = 7.0 Hz, 2H), 2.06 (s, 3H), 1.52-1.47 (m, 2H), 1.31 (t, J = 7.0 Hz, 3H), 1.22-1.16 (m, 2H) LCMS (Analytical Method F): Rt = 3.89 mins; MS (ESIpos) m/z = 475 (M + H)⁺. |
| 161 | 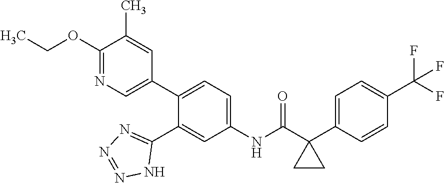 [SMILES: CCOc1ncc(-c2ccc(NC(=O)C3(c4ccc(C(F)(F)F)cc4)CC3)cc2-c2nnn[nH]2)cc1C] | N-[4-(6-ethoxy- 5- methylpyridin- 3-yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.60 (s, 1H), 7.94 (s, 1H), 7.88 (dd, J = 8.5, 2.2 Hz, 1H), 7.72 (d, J = 8.2 Hz, 2H), 7.63 (d, J = 2.3 Hz, 1H), 7.60 (d, J = 8.1 Hz, 2H), 7.47 (d, J = 8.5 Hz, 1H), 7.25-7.17 (m, 1H), 4.30 (q, J = 7.0 Hz, 2H), 2.06 (s, 3H), 1.59- 1.52 (m, 2H), 1.31 (t, J = 7.0 Hz, 3H), 1.25-1.21 (m, 2H). LCMS (Analytical Method F): Rt = 3.98 mins; MS (ESIpos) m/z = 509 (M + H)⁺. |
| 162 | 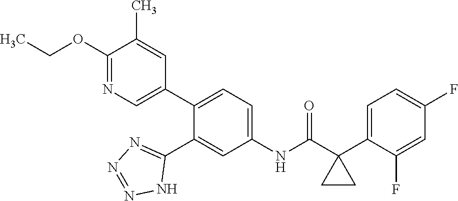 [SMILES: CCOc1ncc(-c2ccc(NC(=O)C3(c4ccc(F)cc4F)CC3)cc2-c2nnn[nH]2)cc1C] | 1-(2,4- difluorophenyl)- N-[4-(6-ethoxy- 5- methylpyridin- 3-yl)-3-(1H- tetrazol-5- yl)phenyl] cyclopropane- carboxamide | ¹H NMR (500 MHz, DMSO-d6) δ [ppm] 9.10 (s, 1H), 7.92-7.81 (m, 2H), 7.62 (d, J = 2.1 Hz, 1H), 7.53 (m, 1H), 7.45 (d, J = 8.9 Hz, 1H), 7.28-7.23 (m, 1H), 7.22 (d, J = 1.6 Hz, 1H), 7.10 (m, 1H), 4.30 (q, J = 7.0 Hz, 2H), 2.06 (s, 3H), 1.59 (m, 2H), 1.31 (t, J = 7.0 Hz, 3H), 1.19-1.12 (m, 2H). LCMS (Analytical Method F): Rt = 3.66 mins; MS (ESIpos) m/z = 477 (M + H)⁺. |
| 163 | 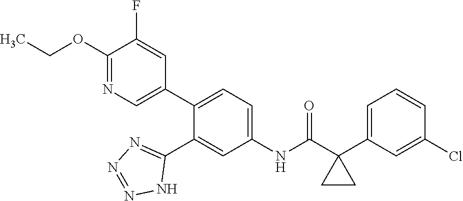 [SMILES: CCOc1ncc(-c2ccc(NC(=O)C3(c4cccc(Cl)c4)CC3)cc2-c2nnn[nH]2)cc1F] | 1-(3- chlorophenyl)- N-[4-(6-ethoxy- 5-fluoropyridin- 3-yl)-3-(1H- tetrazol-5- yl)phenyl] cyclopropane- carboxamide | ¹H NMR (500 MHz, DMSO-d6) δ [ppm] 9.55 (s, 1H), 8.00 (s, 1H), 7.89 (dd, J = 8.5, 2.2 Hz, 1H), 7.64 (d, J = 2.0 Hz, 1H), 7.50 (d, J = 8.5 Hz, 1H), 7.45-7.40 (m, 2H), 7.40-7.34 (m, 3H), 4.38 (q, J = 7.0 Hz, 2H), 1.56-1.45 (m, 2H), 1.34 (t, J = 7.0 Hz, 3H), 1.24-1.13 (m, 2H). LCMS (Analytical Method F): Rt = 3.78 mins; MS (ESIpos) m/z = 479 (M + H)⁺. |
| 164 | 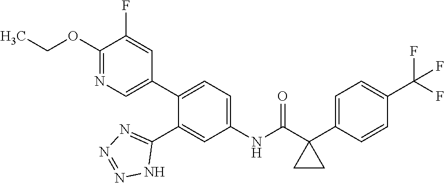 [SMILES: CCOc1ncc(-c2ccc(NC(=O)C3(c4ccc(C(F)(F)F)cc4)CC3)cc2-c2nnn[nH]2)cc1F] | N-[4-(6-ethoxy- 5-fluoropyridin- 3-yl)-3-(1H- tetrazol-5- yl)phenyl]-1-[4- (trifluoromethyl) phenyl] cyclopropane- carboxamide | ¹H NMR (500 MHz, DMSO-d6) δ [ppm] 9.64 (s, 1H), 8.01 (s, 1H), 7.89 (dd, J = 8.5, 2.2 Hz, 1H), 7.72 (d, J = 8.2 Hz, 2H), 7.64 (d, J = 2.0 Hz, 1H), 7.60 (d, J = 8.1 Hz, 2H), 7.51 (d, J = 8.5 Hz, 1H), 7.42 (dd, J = 11.4, 2.0 Hz, 1H), 4.38 (q, J = 7.0 Hz, 2H), 1.60- 1.53 (m, 2H), 1.34 (t, J = 7.0 Hz, 3H), 1.27-1.22 (m, 2H). LCMS (Analytical Method F): Rt = 3.89 mins; MS (ESIpos) m/z = 513 (M + H)⁺. |
| 165 | 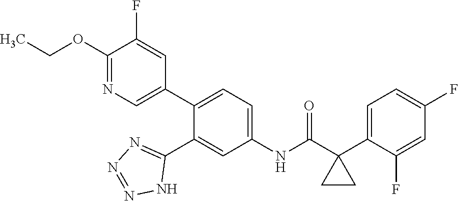 [SMILES: CCOc1ncc(-c2ccc(NC(=O)C3(c4ccc(F)cc4F)CC3)cc2-c2nnn[nH]2)cc1F] | 1-(2,4- difluorophenyl)- N-[4-(6-ethoxy- 5-fluoropyridin- 3-yl)-3-(1H- tetrazol-5- yl)phenyl] cyclopropane- carboxamide | ¹H NMR (500 MHz, DMSO-d6) δ [ppm] 9.14 (s, 1H), 7.94 (s, 1H), 7.88 (dd, J = 8.5, 2.2 Hz, 1H), 7.64 (d, J = 2.0 Hz, 1H),7.56- 7.51 (m, 1H), 7.49 (d, J = 8.6 Hz, 1H), 7.42 (dd, J = 11.4, 2.0 Hz, 1H), 7.25 (td, J = 10.5, 2.6 Hz, 1H), 7.10 (td, J = 8.6, 2.3 Hz, 1H), 4.38 (q, J = 7.0 Hz, 2H), 1.62-1.57 (m, 2H), 1.34 (t, J = 7.0 Hz, 3H), 1.20-1.15 (m, 2H). LCMS (Analytical Method F): Rt = 3.58 mins; MS (ESIpos) m/z = 481 (M + H)⁺. |
| 166 | 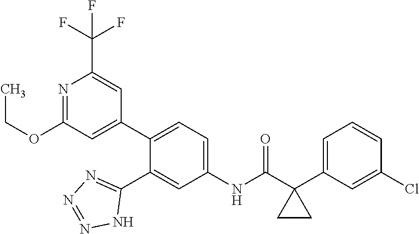 [SMILES: CCOc1cc(-c2ccc(NC(=O)C3(c4cccc(Cl)c4)CC3)cc2-c2nnn[nH]2)cc(C(F)(F)F)n1] | 1-(3- chlorophenyl)- N-{4-[2-ethoxy- 6- (trifluoromethyl) pyridin-4-yl]-3- (1H-tetrazol-5- yl)phenyl} cyclopropane- carboxamide | ¹H NMR (400 MHz, DMSO-d6) δ [ppm] 1.20-1.23 (m, 2H), 1.32 (t, 3H), 1.50-1.53 (m, 2H), 4.32 (q, 2H), 6.83 (s, 1H), 7.01 (d, 1H), 7.37-7.40 (m,3H), 7.45 (m, 1H), 7.59 (d, 1H), 7.93 (dd, 1H), 8.05 (d, 1H), 9.63 (s, 1H). LCMS (method 4): Rt = 0.90 min, m/z = 529 (M + H)⁺ |
| 167 | 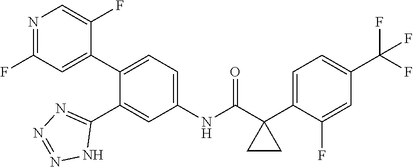 [SMILES: O=C(Nc1ccc(-c2cc(F)ncc2F)c(-c2nnn[nH]2)c1)C1(c2ccc(C(F)(F)F)cc2F)CC1] | N-[4-(2,5- difluoropyridin- 4-yl)-3-(1H- tetrazol-5- yl)phenyl]-1-[2- fluoro-4- (trifluoromethyl) phenyl] cyclopropane- carboxamide | ¹H NMR (400 MHz, DMSO-d6) δ [ppm] 1.27-1.30 (m, 2H), 1.66- 1.69 (m, 2H), 7.28 (dd, 1H), 7.50 (d, 1H), 7.62-7.75 (m, 3H), 7.89 (dd, 1H), 8.13 (s, 1H), 8.15 (d, 1H), 9.38 (s, 1H). LCMS (method 3): Rt = 1.24 min, m/z = 505 (M + H)⁺ |
| 168 | 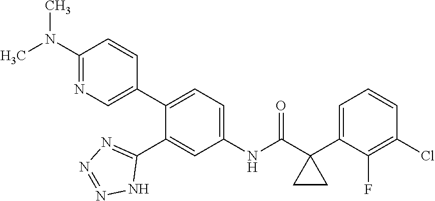 [SMILES: CN(C)c1ccc(-c2ccc(NC(=O)C3(c4cccc(Cl)c4F)CC3)cc2-c2nnn[nH]2)cn1] | 1-(3-chloro-2- fluorophenyl)- N-{4-[6- (dimethylamino) pyridin-3-yl]-3- (1H-tetrazol-5- yl)phenyl} cyclopropane- carboxamide | ¹H NMR (500 MHz, DMSO-d6) δ [ppm] 9.16 (s, 1H), 7.84 (dd, J = 8.5, 2.3 Hz, 1H), 7.82-7.80 (m, 2H), 7.59-7.53 (m, 1H), 7.45 (t, J = 7.3 Hz, 2H), 7.27-7.21 (m, 1H), 7.10 (dd, J = 8.8, 2.5 Hz, 1H), 6.54 (d, J = 8.9 Hz, 1H), 3.00 (s, 6H), 1.69-1.56 (m, 2H), 1.28-1.13 (m, 2H). LCMS (Analytical Method F): Rt = 2.26 mins; MS (ESIpos) m/z = 478.1 (M + H)⁺. |
| 169 | 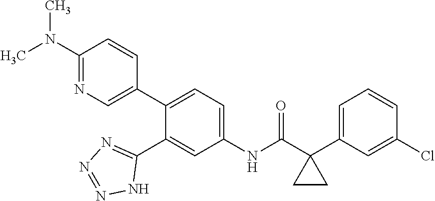 [SMILES: CN(C)c1ccc(-c2ccc(NC(=O)C3(c4cccc(Cl)c4)CC3)cc2-c2nnn[nH]2)cn1] | 1-(3- chlorophenyl)- N-{4-[6- (dimethylamino) pyridin-3-yl]-3- (1H-tetrazol-5- yl)phenyl} cyclopropane- carboxamide | ¹H NMR (500 MHz, DMSO-d6) δ [ppm] 9.47 (s, 1H), 7.87 (d, J = 2.2 Hz, 1H), 7.85 (dd, J = 8.5, 2.3 Hz, 1H), 7.81 (d, J = 2.5 Hz, 1H), 7.46-7.42 (m, 2H), 7.41- 7.34 (m, 3H), 7.09 (dd, J = 8.8, 2.5 Hz, 1H), 6.53 (d, J = 8.9 Hz, 1H), 3.00 (s, 6H), 1.54-1.44 (m, 2H), 1.25-1.12 (m, 2H). LCMS (Analytical Method F): Rt = 2.27 mins; MS (ESIpos) m/z = 460.1 (M + H)⁺. |
| 170 | 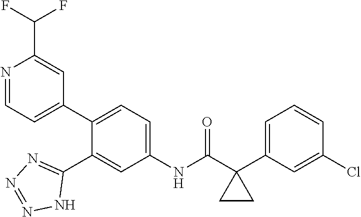 [SMILES: O=C(Nc1ccc(-c2ccnc(C(F)F)c2)c(-c2nnn[nH]2)c1)C1(c2cccc(Cl)c2)CC1] | 1-(3- chlorophenyl)- N-{4-[2- (difluoromethyl) pyridin-4-yl]-3- (1H-tetrazol-5- yl)phenyl} cyclopropane- carboxamide | ¹H NMR (500 MHz, DMSO-d6) δ [ppm] 9.61 (s, 1H), 8.58 (d, J = 5.1 Hz, 1H), 8.05 (s, 1H), 7.98- 7.91 (m, 1H), 7.60 (d, J = 8.5 Hz, 1H), 7.47-7.44 (m, 1H), 7.43- 7.31 (m, 4H), 7.23 (d, J = 4.8 Hz, 1H), 6.91 (t, J = 54.9 Hz, 1H), 1.57-1.44 (m, 2H), 1.24-1.16 (m, 2H). LCMS (Analytical Method F): Rt = 3.39 mins, MS (ESIpos): m/z = 467 (M + H)+ |
| 171 | 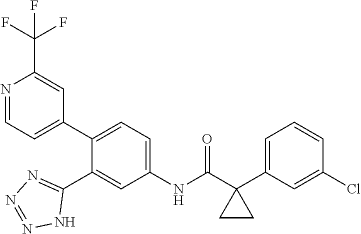 [SMILES: O=C(Nc1ccc(-c2ccnc(C(F)(F)F)c2)c(-c2nnn[nH]2)c1)C1(c2cccc(Cl)c2)CC1] | 1-(3- chlorophenyl)- N-{3-(1H- tetrazol-5-yl)-4- [2- (trifluoromethyl) pyridin-4- yl]phenyl} cyclopropane- carboxamide | ¹H NMR (500 MHz, DMSO-d6) δ [ppm] 9.63 (s, 1H), 8.66 (d, J = 5.0 Hz, 1H), 8.13-8.05 (m, 1H), 7.96 (dd, J = 8.6, 2.1 Hz, 1H), 7.64 (d, J = 8.5 Hz, 1H), 7.56 (s, 1H), 7.47-7.44 (m, 1H), 7.43- 7.34 (m, 4H), 1.55-1.49 (m, 2H), 1.26-1.19 (m, 2H). LCMS (Analytical Method F): Rt = 3.68 mins; MS (ESIpos) m/z = 485.0 (M + H)⁺. |
| 172 | 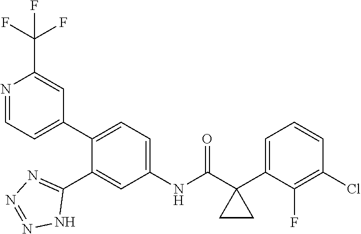 [SMILES: O=C(Nc1ccc(-c2ccnc(C(F)(F)F)c2)c(-c2nnn[nH]2)c1)C1(c2cccc(Cl)c2F)CC1] | 1-(3-chloro-2- fluorophenyl)- N-{3-(1H- tetrazol-5-yl)-4- [2- (trifluoromethyl) pyridin-4- yl]phenyl} cyclopropane- carboxamide | ¹H NMR (500 MHz, DMSO-d6) δ [ppm] 9.32 (s, 1H), 8.66 (d, J = 5.1 Hz, 1H), 8.05 (s, 1H), 7.94 (dd, J = 8.6, 2.1 Hz, 1H), 7.63 (d, J = 8.5 Hz, 1H), 7.60-7.54 (m, 2H), 7.49-7.43 (m, 1H), 7.37 (d, J = 4.6 Hz, 1H), 7.28-7.22 (m, 1H), 1.70-1.60 (m, 2H), 1.30- 1.18 (m, 2H). LCMS (Analytical Method F): Rt = 3.57 mins; MS (ESIpos) m/z = 503.0 (M + H)⁺. |
| 173 | 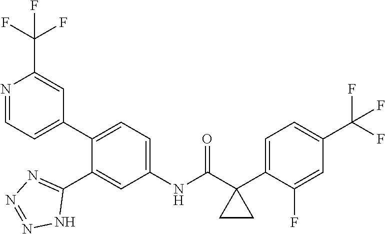 [SMILES: O=C(Nc1ccc(-c2ccnc(C(F)(F)F)c2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[2- (trifluoromethyl) pyridin-4- yl]phenyl} cyclopropane- carboxamide | ¹H NMR (400 MHz, DMSO-d6) δ [ppm] 1.26-1.29 (m, 2H), 1.66- 1.69 (m, 2H), 7.37 (dd, 1H), 7.56- 7.63 (m, 3H), 7.69-7.74 (m, 2H), 7.92 (dd, 1H), 8.02 (d, 1H), 8.65 (d, 1H), 9.35 (s, 1H). LCMS (method 1): Rt = 1.30 min, m/z = 537 (M + H)⁺ |
| 174 | 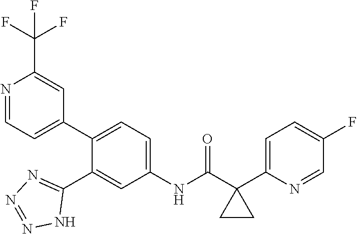 [SMILES: O=C(Nc1ccc(-c2ccnc(C(F)(F)F)c2)c(-c2nnn[nH]2)c1)C1(c2ccc(F)cn2)CC1] | 1-(5- fluoropyridin-2- yl)-N-{3-(1H- tetrazol-5-yl)-4- [2- (trifluoromethyl) pyridin-4- yl]phenyl} cyclopropane- carboxamide | ¹H NMR (400 MHz, DMSO-d6) δ [ppm] 1.37-1.40 (m, 2H), 1.53- 1.56 (m, 2H), 7.38 (dd, 1H), 7.47 (dd, 1H), 7.56 (s, 2H), 7.61 (d, 1H), 7.72 (dt, 1H), 7.97 (dd, 1H), 8.12 (d, 1H), 8.55 (d, 1H), 8.66 (d, 1H), 10.30 (s, 1H). LCMS (method 4): Rt = 0.67 min, m/z = 470 (M + H)⁺ |
| 175 | 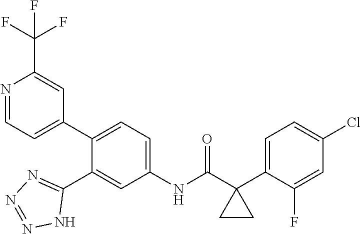 [SMILES: O=C(Nc1ccc(-c2ccnc(C(F)(F)F)c2)c(-c2nnn[nH]2)c1)C1(c2ccc(Cl)cc2F)CC1] | 1-(4-chloro-2- fluorophenyl)- N-{3-(1H- tetrazol-5-yl)-4- [2- (trifluoromethyl) pyridin-4- yl]phenyl} cyclopropane- carboxamide | LCMS (method 1): Rt = 1.24 min, m/z = 503 (M + H)⁺ |
| 176 | 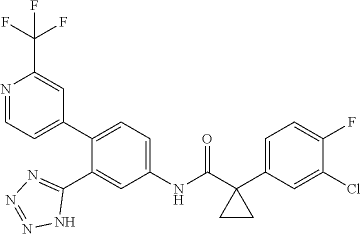 [SMILES: O=C(Nc1ccc(-c2ccnc(C(F)(F)F)c2)c(-c2nnn[nH]2)c1)C1(c2ccc(F)c(Cl)c2)CC1] | 1-(3-chloro-4- fluorophenyl)- N-{3-(1H- tetrazol-5-yl)-4- [2- (trifluoromethyl) pyridin-4- yl]phenyl} cyclopropane- carboxamide | LCMS (method 1): Rt = 1.25 min, m/z = 503 (M + H)⁺ |
| 177 | 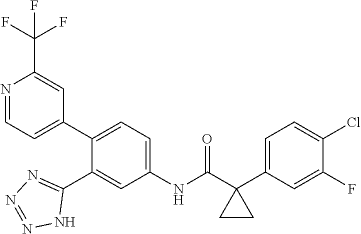 [SMILES: O=C(Nc1ccc(-c2ccnc(C(F)(F)F)c2)c(-c2nnn[nH]2)c1)C1(c2ccc(Cl)c(F)c2)CC1] | 1-(4-chloro-3- fluorophenyl)- N-{3-(1H- tetrazol-5-yl)-4- [2- (trifluoromethyl) pyridin-4- yl]phenyl} cyclopropane- carboxamide | LCMS (method 1): Rt = 1.26 min, m/z = 503 (M + H)⁺ |
| 178 | 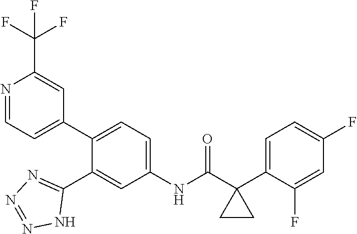 [SMILES: O=C(Nc1ccc(-c2ccnc(C(F)(F)F)c2)c(-c2nnn[nH]2)c1)C1(c2ccc(F)cc2F)CC1] | 1-(2,4- difluorophenyl)- N-{3-(1H- tetrazol-5-yl)-4- [2- (trifluoromethyl) pyridin-4- yl]phenyl} cyclopropane- carboxamide | LCMS (method 1): Rt = 1.17 min, m/z = 487 (M + H)⁺ |
| 179 | 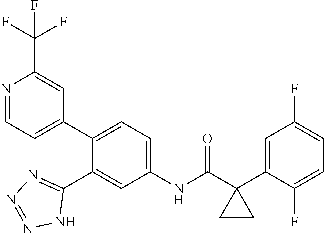 [SMILES: O=C(Nc1ccc(-c2ccnc(C(F)(F)F)c2)c(-c2nnn[nH]2)c1)C1(c2cc(F)ccc2F)CC1] | 1-(2,5- difluorophenyl)- tetrazol-5-yl)-4- [2- (trifluoromethyl) pyridin-4- yl]phenyl} cyclopropane- carboxamide | LCMS (method 2): Rt = 1.14 min, m/z = 487 (M + H)⁺ |
| 180 | 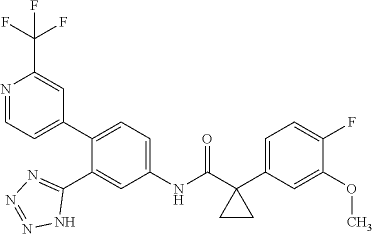 [SMILES: COc1cc(C2(C(=O)Nc3ccc(-c4ccnc(C(F)(F)F)c4)c(-c4nnn[nH]4)c3)CC2)ccc1F] | 1-(4-fluoro-3- methoxyphenyl)- N-{3-(1H- tetrazol-5-yl)-4- [2- (trifluoromethyl) pyridin-4- yl]phenyl} cyclopropane- carboxamide | LCMS (method 1): Rt = 1.17 min, m/z = 499 (M + H)⁺ |
| 181 | 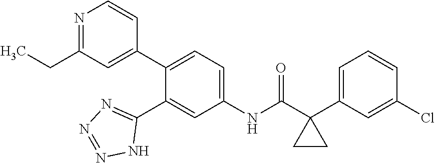 [SMILES: CCc1cc(-c2ccc(NC(=O)C3(c4cccc(Cl)c4)CC3)cc2-c2nnn[nH]2)ccn1] | 1-(3- chlorophenyl)- N-[4-(2- ethylpyridin-4- yl)-3-(1H- tetrazol-5- yl)phenyl] cyclopropane- carboxamide | ¹H NMR (400 MHz, DMSO-d6) δ [ppm] 1.13 (t, 3H), 1.20-1.22 (m, 2H), 1.49-1.52 (m, 2H), 2.66 (q, 2H), 6.86 (dd, 1H), 6.90 (s, 1H), 7.36-7.41 (m, 3H), 7.45- 7.46 (m, 1H), 7.55 (d, 1H), 7.93 (dd, 1H), 7.98 (d, 1H), 8.36 (d, 1H), 9.58 (s, 1H). LCMS (method 4): Rt = 0.75 min, m/z = 445 (M + H)⁺ |
| 182 | 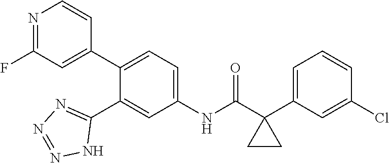 [SMILES: O=C(Nc1ccc(-c2ccnc(F)c2)c(-c2nnn[nH]2)c1)C1(c2cccc(Cl)c2)CC1] | 1-(3- chlorophenyl)- N-[4-(2- fluoropyridin-4- yl)-3-(1H- tetrazol-5- yl)phenyl] cyclopropane- carboxamide | ¹H NMR (400 MHz, DMSO-d6) δ [ppm] 1.19-1.22 (m, 2H), 1.49- 1.52 (m, 2H), 6.93-6.94 (m, 2H), 7.36-7.41 (m, 3H), 7.45 (m, 1H), 7.54 (d, 1H), 7.92 (dd, 1H), 8.01 (d, 1H), 8.10 (d, 1H), 9.61 (s, 1H). LCMS (method 3): Rt = 1.19 min, m/z = 435 (M + H)⁺ |
| 183 | 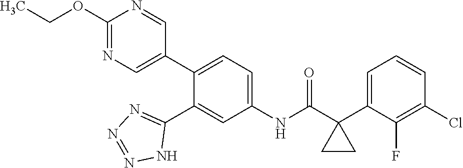 [SMILES: CCOc1ncc(-c2ccc(NC(=O)C3(c4cccc(Cl)c4F)CC3)cc2-c2nnn[nH]2)cn1] | 1-(3-chloro-2- fluorophenyl)- N-[4-(2- ethoxypyrimidin- 5-yl)-3-(1H- tetrazol-5- yl)phenyl] cyclopropane- carboxamide | ¹H NMR (500 MHz, DMSO-d6) δ [ppm] 9.25 (s, 1H), 8.32 (s, 2H), 8.02 (s, 1H), 7.88 (dd, J = 8.5, 2.2 Hz, 1H), 7.60-7.54 (m, 1H), 7.52 (d, J = 8.5 Hz, 1H), 7.48- 7.43 (m, 1H), 7.28-7.22 (m, 1H), 4.35 (q, J = 7.0 Hz, 2H), 1.69-1.59 (m, 2H), 1.33 (t, J = 7.0 Hz, 3H), 1.28-1.17 (m, 2H). LCMS (Analytical Method A): Rt = 3.30 mins; MS (ESIpos) m/z = 480.0 (M + H)⁺. |
| 184 | 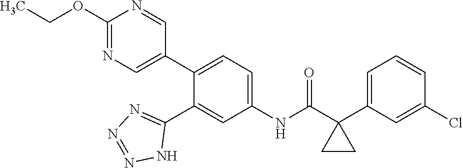 [SMILES: CCOc1ncc(-c2ccc(NC(=O)C3(c4cccc(Cl)c4)CC3)cc2-c2nnn[nH]2)cn1] | 1-(3- chlorophenyl)- N-[4-(2- ethoxypyrimidin- 5-yl)-3-(1H- tetrazol-5- yl)phenyl] cyclopropane- carboxamide | ¹H NMR (500 MHz, DMSO-d6) δ [ppm] 9.57 (s, 1H), 8.32 (s, 2H), 8.11-8.03 (m, 1H), 7.90 (dd, J = 8.5, 2.2 Hz, 1H), 7.52 (d, J = 8.5 Hz, 1H), 7.46-7.43 (m, 1H), 7.42-7.34 (m, 3H), 4.35 (q, J = 7.0 Hz, 2H), 1.53-1.48 (m, 2H), 1.34 (t, J = 7.0 Hz, 3H), 1.23- 1.18 (m, 2H). LCMS (Analytical Method D): Rt = 4.30 mins; MS (ESIpos) m/z = 462.0 (M + H)⁺. |
| 185 | 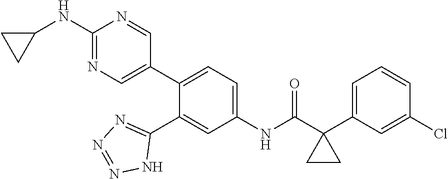 [SMILES: O=C(Nc1ccc(-c2cnc(NC3CC3)nc2)c(-c2nnn[nH]2)c1)C1(c2cccc(Cl)c2)CC1] | 1-(3- chlorophenyl)- N-{4-[2- (cyclopropylamino) pyrimidin-5- yl]-3-(1H- tetrazol-5- yl)phenyl} cyclopropane- carboxamide | LCMS (method 3): Rt = 1.10 min, m/z = 473 (M + H)⁺ |
| 186 | 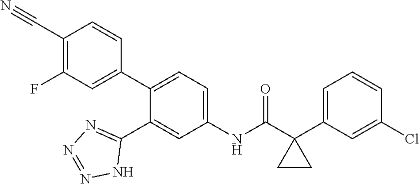 [SMILES: N#Cc1ccc(-c2ccc(NC(=O)C3(c4cccc(Cl)c4)CC3)cc2-c2nnn[nH]2)cc1F] | 1-(3- chlorophenyl)- N-[4′-cyano-3′- fluoro-2-(1H- tetrazol-5- yl)biphenyl-4- yl]cyclopropane carboxamide | ¹H NMR (400 MHz, DMSO-d6) δ [ppm] 1.20-1.23 (m, 2H), 1.49- 1.52 (m, 2H), 7.01 (dd, 1H), 7.34- 7.41 (m, 4H), 7.44-7.45 (m, 1H), 7.53 (d, 1H), 7.83 (dd, 1H), 7.92 (dd, 1H), 8.03 (d, 1H), 9.61 (s, 1H). LCMS (method 2): Rt = 1.21 min, m/z = 459 (M + H)⁺ |
| 187 | 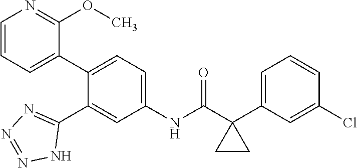 [SMILES: COc1ncccc1-c1ccc(NC(=O)C2(c3cccc(Cl)c3)CC2)cc1-c1nnn[nH]1] | 1-(3- chlorophenyl)- N-[4-(2- methoxypyridin- 3-yl)-3-(1H- tetrazol-5- yl)phenyl] cyclopropane- carboxamide | ¹H NMR (400 MHz, DMSO-d6) δ [ppm] 1.19-1.22 (m, 2H), 1.48- 1.52 (m, 2H), 3.40 (s, 3H), 7.03 (dd, 1H), 7.36-7.41 (m, 4H), 7.45 (m, 1H), 7.56 (dd, 1H), 7.83 (dd, 1H), 8.01 (d, 1H), 8.10 (dd, 1H), 9.54 (s, 1H). UPLC (acid): Rt = 1.19 min, m/z = 447 (M + H)⁺ |
| 189 | 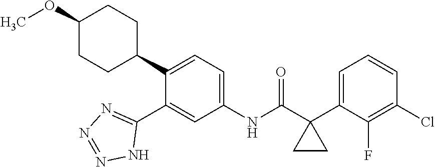 [SMILES: CO[C@H]1CC[C@@H](c2ccc(NC(=O)C3(c4cccc(Cl)c4F)CC3)cc2-c2nnn[nH]2)CC1] | 1-(3-chloro-2- fluorophenyl)- N-[4-(cis-4- methoxy- cyclohexyl)- 3-(1H- tetrazol-5- yl)phenyl] cyclopropane- carboxamide | ¹H NMR (500 MHz, DMSO-d6) δ [ppm] 9.01 (s, 1H), 7.72-7.63 (m, 2H), 7.55 (t, J = 6.9 Hz, 1H), 7.43 (t, J = 6.6 Hz, 1H), 7.38 (d, J = 8.6 Hz, 1H), 7.23 (t, J = 7.9 Hz, 1H), 3.44-3.37 (m, 1H), 3.22 (s, 3H), 2.88-2.72 (m, 1H), 1.95-1.85 (m, 2H), 1.65 (q, J = 13.0, 11.6 Hz, 2H), 1.60-1.55 (m, 2H), 1.47-1.38 (m, 2H), 1.37-1.26 (m, 2H), 1.20-1.15 (m, 2H). LCMS (Analytical Method F): Rt = 3.55 mins; MS (ESIpos) m/z = 470 (M + H)⁺. |
| 190 | 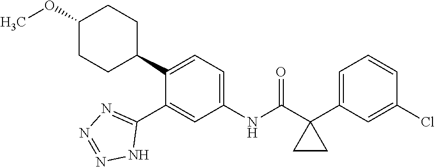 [SMILES: CO[C@H]1CC[C@H](c2ccc(NC(=O)C3(c4cccc(Cl)c4)CC3)cc2-c2nnn[nH]2)CC1] | 1-(3- chlorophenyl)- N-[4-(trans-4- methoxy- cyclohexyl)- 3-(1H- tetrazol-5- yl)phenyl] cyclopropane- carboxamide | ¹H NMR (500 MHz, DMSO-d6) δ [ppm] 9.30 (s, 1H), 7.74 (d, J = 2.3 Hz, 1H), 7.68 (dd, J = 8.6, 2.2 Hz, 1H), 7.43-7.33 (m, 5H), 3.22 (s, 3H), 3.16-3.11 (m, 1H), 2.87-2.78 (m, 1H), 2.06-2.00 (m, 2H), 1.75-1.69 (m, 2H), 1.53-1.42 (m, 4H), 1.18-1.14 (m, 2H), 1.11-1.01 (m, 2H). LCMS (Analytical Method F): Rt = 3.58 mins; MS (ESIpos) m/z = 452 (M + H)⁺. |
| 191 | 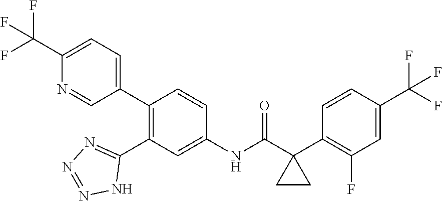 [SMILES: O=C(Nc1ccc(-c2ccc(C(F)(F)F)nc2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.27-1.30 (m, 2H), 1.66- 1.69 (m, 2H), 7.58-7.65 (m, 2H), 7.70-7.77 (m, 3H), 7.86 (d, 1H), 7.94 (d, 1H), 8.05 (s, br, 1H), 8.58 (d, 1H), 9.37 (s, 1H). LCMS (method 1): Rt = 1.30 min, m/z = 537 (M + H)⁺ |
| 192 | 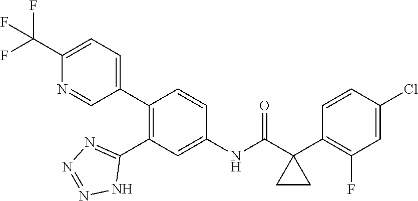 [SMILES: O=C(Nc1ccc(-c2ccc(C(F)(F)F)nc2)c(-c2nnn[nH]2)c1)C1(c2ccc(Cl)cc2F)CC1] | 1-(4-chloro-2- fluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.18-1.21 (m, 2H), 1.60- 1.63 (m, 2H), 7.33 (dd, 1H), 7.35- 7.40 (m, 1H), 7.46 (dd, 1H), 7.50-7.55 (m, 1H), 7.76 (dd, 1H), 7.84 (d, 1H), 7.91 (dd, 1H), 8.03 (d, 1H), 8.51 (d, 1H), 9.26 (s, 1H). LCMS (method 1): Rt = 1.26 min, m/z = 503 (M + H)⁺ |
| 193 | 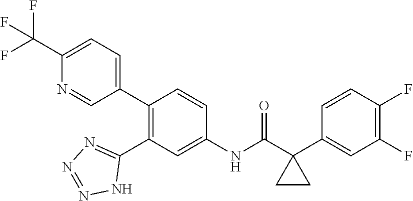 [SMILES: O=C(Nc1ccc(-c2ccc(C(F)(F)F)nc2)c(-c2nnn[nH]2)c1)C1(c2ccc(F)c(F)c2)CC1] | 1-(3,4- difluorophenyl)- N-{3-(1H- tetrazol-5-yl)-4- [6- (trifluoromethyl) pyridin-3- yl]phenyl} cyclopropane- carboxamide | ¹H NMR (400 MHz, DMSO-d6) δ [ppm] 1.18-1.21 (m, 2H), 1.50- 1.53 (m, 2H), 7.27-7.31 (m, 1H), 7.40-7.54 (m, 3H), 7.75 (dd, 1H), 7.82 (d, 1H), 7.89 (dd, 1H), 8.04 (d, 1H), 8.48 (d, 1H), 9.36 (s, 1H). LCMS (method 1): Rt = 1.22 min, m/z = 487 (M + H)⁺ |
| 194 | 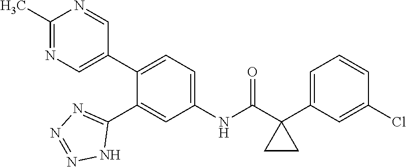 [SMILES: Cc1ncc(-c2ccc(NC(=O)C3(c4cccc(Cl)c4)CC3)cc2-c2nnn[nH]2)cn1] | 1-(3- chlorophenyl)- N-[4-(2- methylpyrimidin- 5-yl)-3-(1H- tetrazol-5- yl)phenyl] cyclopropane- carboxamide | ¹H NMR (500 MHz, DMSO-d6) δ [ppm] 9.59 (s, 1H), 8.41 (s, 2H), 8.08 (d, J = 1.8 Hz, 1H), 7.94- 7.87 (m, 1H), 7.54 (d, J = 8.5 Hz, 1H), 7.45 (d, J = 1.7 Hz, 1H), 7.42-7.32 (m, 3H), 2.62 (s, 3H), 1.54-1.47 (m, 2H), 1.23-1.18 (m, 2H). LCMS (Analytical Method F): Rt = 2.92 mins; MS (ESIpos) m/z = 432 (M + H)⁺. |
| 195 | 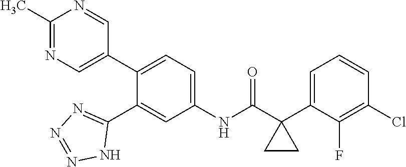 [SMILES: Cc1ncc(-c2ccc(NC(=O)C3(c4cccc(Cl)c4F)CC3)cc2-c2nnn[nH]2)cn1] | 1-(3-chloro-2- fluorophenyl)- N-[4-(2- methylpyrimidin- 5-yl)-3-(1H- tetrazol-5- yl)phenyl] cyclopropane- carboxamide | ¹H NMR (500 MHz, DMSO-d6) δ [ppm] 9.27 (s, 1H), 8.42 (s, 2H), 8.04 (s, 1H), 7.90 (dd, J = 8.5, 2.2 Hz, 1H), 7.60-7.51 (m, 2H), 7.49-7.43 (m, 1H), 7.28-7.21 (m, 1H), 2.62 (s, 3H), 1.68-1.60 (m, 2H), 1.27-1.18 (m, 2H). LCMS (Analytical Method F): Rt = 2.88 mins; MS (ESIpos) m/z = 450 (M + H)⁺. |
| 196 | 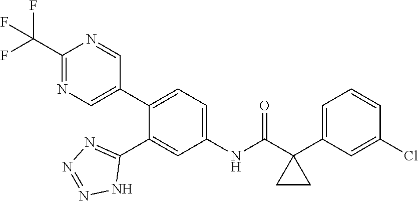 [SMILES: O=C(Nc1ccc(-c2cnc(C(F)(F)F)nc2)c(-c2nnn[nH]2)c1)C1(c2cccc(Cl)c2)CC1] | 1-(3- chlorophenyl)- N-{3-(1H- tetrazol-5-yl)-4- [2- (trifluoromethyl) pyrimidin-5- yl]phenyl} cyclopropane- carboxamide | ¹H NMR (500 MHz, DMSO-d6) δ [ppm] 9.67 (s, 1H), 8.87 (s, 2H), 8.24 (s, 1H), 7.97 (dd, J = 8.5, 2.2 Hz, 1H), 7.65 (d, J = 8.5 Hz, 1H), 7.46 (d, J = 1.6 Hz, 1H), 7.43-7.35 (m, 3H), 1.57-1.47 (m, 2H), 1.28-1.17 (m, 2H). LCMS (Analytical Method D): Rt = 4.43 mins; MS (ESIpos) m/z = 486.0 (M + H)⁺. |
| 197 | 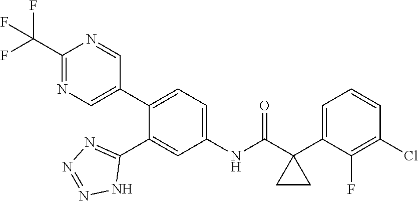 [SMILES: O=C(Nc1ccc(-c2cnc(C(F)(F)F)nc2)c(-c2nnn[nH]2)c1)C1(c2cccc(Cl)c2F)CC1] | 1-(3-chloro-2- fluorophenyl)- N-{3-(1H- tetrazol-5-yl)-4- [2- (trifluoromethyl) pyrimidin-5- yl]phenyl} cyclopropane- carboxamide | ¹H NMR (500 MHz, DMSO-d6) δ [ppm] 9.35 (s, 1H), 8.88 (s, 2H), 8.19 (s, 1H), 7.94 (dd, J = 8.5, 2.2 Hz, 1H), 7.64 (d, J = 8.5 Hz, 1H), 7.61-7.55 (m, 1H), 7.50- 7.44 (m, 1H), 7.29-7.23 (m, 1H), 1.71-1.61 (m, 2H), 1.31- 1.19 (m, 2H). LCMS (Analytical Method F): Rt = 3.51 mins; MS (ESIpos) = m/z = 504.0 (M + H)⁺. |
| 198 | 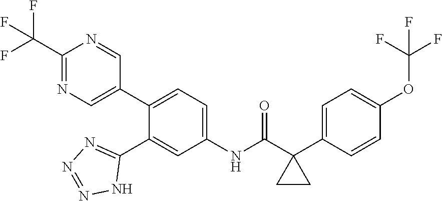 [SMILES: O=C(Nc1ccc(-c2cnc(C(F)(F)F)nc2)c(-c2nnn[nH]2)c1)C1(c2ccc(OC(F)(F)F)cc2)CC1] | N-{3-(1H- tetrazol-5-yl)-4- [2- (trifluoromethyl) pyrimidin-5- yl]phenyl}-1-[4- (trifluoromethoxy) phenyl] cyclopropane- carboxamide | ¹H NMR (250 MHz, DMSO-d6) δ [ppm] 1.13-1.30 (m, 2H), 1.48- 1.60 (m, 2H), 7.29-7.42 (m, 2H), 7.48-7.59 (m, 2H), 7.65 (d, J = 8.5 Hz, 1H), 7.96 (dd, J = 8.5, 2.2 Hz, 1H), 8.24 (d, J = 1.9 Hz, 1H), 8.87 (s, 2H), 9.67 (s, 1H). LCMS (Analytical Method D): Rt = 4.65 mins; MS (ESIpos) m/z = 536.05 (M + H)⁺. |
| 199 | 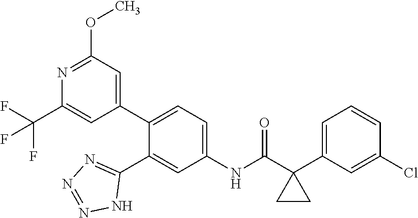 [SMILES: COc1cc(-c2ccc(NC(=O)C3(c4cccc(Cl)c4)CC3)cc2-c2nnn[nH]2)cc(C(F)(F)F)n1] | 1-(3- chlorophenyl)- N-{4-[2- methoxy-6- (trifluoromethyl) pyridin-4-yl]-3- (1H-tetrazol-5- yl)phenyl} cyclopropane- carboxamide | ¹H NMR (500 MHz, DMSO-d6) δ [ppm] 9.56 (s, 1H), 8.02-7.96 (m, 1H), 7.92-7.82 (m, 1H), 7.54-7.46 (m, 1H), 7.46-7.32 (m, 4H), 7.03 (s, 1H), 6.83 (s, 1H), 3.88 (s, 3H), 1.56-1.46 (m, 2H), 1.26-1.12 (m, 2H). LCMS (Analytical Method F): Rt = 4.04 mins; MS (ESIpos) m/z = 515 (M + H)⁺. |
| 200 | 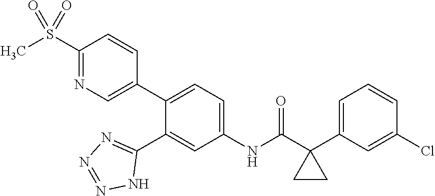 [SMILES: CS(=O)(=O)c1ccc(-c2ccc(NC(=O)C3(c4cccc(Cl)c4)CC3)cc2-c2nnn[nH]2)cn1] | 1-(3- chlorophenyl)- N-{4-[6- (methylsulfonyl) pyridin-3-yl]-3- (1H-tetrazol-5- yl)phenyl} cyclopropane- carboxamide | ¹H NMR (500 MHz, DMSO-d6) δ [ppm] 9.65 (s, 1H), 8.54 (d, J = 1.7 Hz, 1H), 8.12 (s, 1H), 8.00- 7.92 (m, 2H), 7.81 (dd, J = 8.1, 1.9 Hz, 1H), 7.60 (d, J = 8.5 Hz, 1H), 7.45 (d, J = 1.7 Hz, 1H), 7.43-7.34 (m, 3H), 3.30 (s, 3H), 1.54-1.48 (m, 2H), 1.24-1.18 (m, 2H). LCMS (Analytical Method F): Rt = 3.11 mins; m/z = 495 (M + H)⁺. |
| 201 | 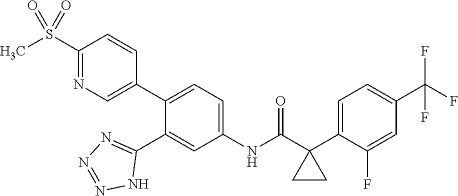 [SMILES: CS(=O)(=O)c1ccc(-c2ccc(NC(=O)C3(c4ccc(C(F)(F)F)cc4F)CC3)cc2-c2nnn[nH]2)cn1] | 1-[2-fluoro-4- (trifluoromethyl) phenyl]-N-{4- [6- (methylsulfonyl) pyridin-3-yl]-3- (1H-tetrazol-5- yl)phenyl} cyclopropane- carboxamide | ¹H NMR (500 MHz, DMSO-d6) δ [ppm] 9.36 (s, 1H), 8.54 (d, J = 2.1 Hz, 1H), 8.06 (s, 1H), 7.97 (d, J = 8.1 Hz, 1H), 7.94 (dd, J = 8.5, 2.2 Hz, 1H), 7.81 (dd, J = 8.1, 2.0 Hz, 1H), 7.73-7.67 (m, 2H), 7.62 (d, J = 8.2 Hz, 1H), 7.59 (d, J = 8.5 Hz, 1H), 3.29 (s, 3H), 1.71-1.64 (m, 2H), 1.31- 1.25 (m, 2H). LCMS (Analytical Method F): Rt = 3.28 mins; MS (ESIpos) m/z = 547 (M + H)⁺. |
| 202 | 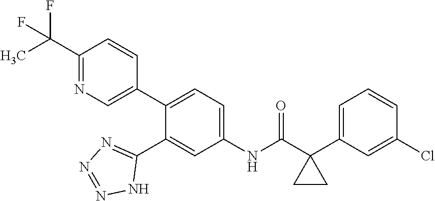 [SMILES: CC(F)(F)c1ccc(-c2ccc(NC(=O)C3(c4cccc(Cl)c4)CC3)cc2-c2nnn[nH]2)cn1] | 1-(3- chlorophenyl)- N-{4-[6-(1,1- difluoroethyl) pyridin-3-yl]-3- (1H-tetrazol-5- yl)phenyl} cyclopropane- carboxamide | ¹H NMR (250 MHz, DMSO-d6) δ [ppm] 1.16-1.24 (m, 2H), 1.43- 1.57 (m, 2H), 2.00 (t, J = 19.1 Hz, 3H), 7.31-7.40 (m, 3H), 7.42-7.48 (m, 1H), 7.56 (d, J = 8.5 Hz, 1H), 7.63 (d, J = 1.3 Hz, 2H), 7.93 (dd, J = 8.5, 2.2 Hz, 1H), 8.05 (d, J = 2.0 Hz, 1H), 8.29-8.48 (m, 1H), 9.59 (s, 1H). LCMS (Analytical Method D): Rt = 4.59 mins; MS (ESIpos) m/z = 481.15 (M + H)⁺. |
| 203 | 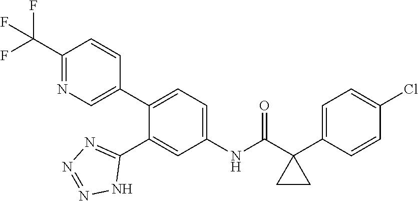 [SMILES: O=C(Nc1ccc(-c2ccc(C(F)(F)F)nc2)c(-c2nnn[nH]2)c1)C1(c2ccc(Cl)cc2)CC1] | 1-(4- chlorophenyl)- N-{3-(1H- tetrazol-5-yl)-4- [6- (trifluoromethyl) pyridin-3- yl]phenyl} cyclopropane- carboxamide | ¹H NMR (500 MHz, DMSO-d6) δ [ppm] 9.34 (s, 1H), 7.79 (s, 1H), 7.71 (d, J = 7.6 Hz, 1H), 7.47- 7.40 (m, 2H), 7.39-7.32 (m, 3H), 4.10-3.95 (m, 2H), 3.04- 2.92 (m, 1H), 2.71-2.58 (m, 2H), 1.70-1.62 (m, 2H), 1.52- 1.44 (m, 4H), 1.40 (s, 9H), 1.20- 1.14 (m, 2H). LCMS (Analytical Method F): Rt = 3.71 mins; MS (ESIpos) m/z = 485 (M + H)⁺. |
| 204 | 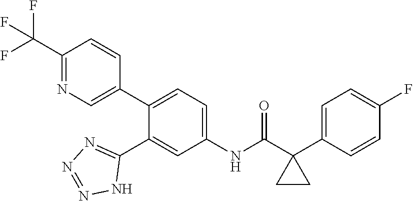 [SMILES: O=C(Nc1ccc(-c2ccc(C(F)(F)F)nc2)c(-c2nnn[nH]2)c1)C1(c2ccc(F)cc2)CC1] | 1-(4- fluorophenyl)- N-{3-(1H- tetrazol-5-yl)-4- [6- (trifluoromethyl) pyridin-3- yl]phenyl} cyclopropane- carboxamide | ¹H NMR (500 MHz, DMSO-d6) δ [ppm] 9.41 (s, 1H), 8.51 (d, J = 2.0 Hz, 1H), 8.09 (d, J = 1.9 Hz, 1H), 7.95 (dd, J = 8.5, 2.2 Hz, 1H), 7.84 (d, J = 8.1 Hz, 1H), 7.74 (dd, J = 8.1, 1.9 Hz, 1H), 7.58 (d, J = 8.5 Hz, 1H), 7.49- 7.44 (m, 2H), 7.22-7.16 (m, 2H), 1.53-1.49 (m, 2H), 1.18- 1.13 (m, 2H). LCMS (Analytical Method F): Rt = 3.48 mins; MS (ESIpos) m/z = 469 (M + H)⁺. |
| 205 | 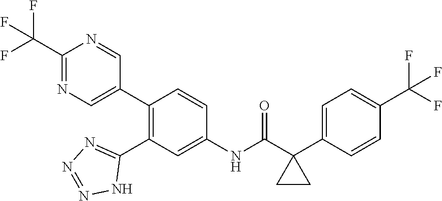 [SMILES: O=C(Nc1ccc(-c2cnc(C(F)(F)F)nc2)c(-c2nnn[nH]2)c1)C1(c2ccc(C(F)(F)F)cc2)CC1] | N-{3-(1H- tetrazol-5-yl)-4- [2- (trifluoromethyl) pyrimidin-5- yl]phenyl)-1-[4- (trifluoromethyl) phenyl] cyclopropane- carboxamide | ¹H NMR (500 MHz, DMSO-d6) δ [ppm] 1.25-1.28 (m, 2H), 1.56- 1.61 (m, 2H), 7.62 (t, J = 7.9 Hz, 3H), 7.73 (d, J = 8.3 Hz, 2H), 7.96 (dd, J = 8.5, 2.2 Hz, 1H), 8.24 (d, J = 1.9 Hz, 1H), 8.86 (s, 2H), 9.76 (s, 1H). LCMS (Analytical Method F): Rt = 3.77 mins; MS (ESIpos) m/z = 520.1 (M + H)⁺. |
| 206 | 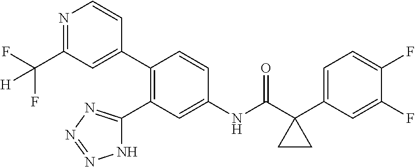 [SMILES: O=C(Nc1ccc(-c2ccnc(C(F)F)c2)c(-c2nnn[nH]2)c1)C1(c2ccc(F)c(F)c2)CC1] | N-{4-[2- (difluoromethyl) pyridin-4-yl]-3- (1H-tetrazol-5- yl)phenyl}-1- (3,4- difluorophenyl) cyclopropane- carboxamide | ¹H NMR (500 MHz, DMSO-d6) δ [ppm] 9.22 (s, 1H), 8.45 (d, J = 5.1 Hz, 1H), 7.85 (d, J = 2.3 Hz, 1H), 7.79-7.74 (m, 1H), 7.52- 7.45 (m, 1H), 7.45-7.36 (m, 1H), 7.36-7.33 (m, 1H), 7.33- 7.25 (m, 2H), 7.19-7.12 (m, 1H), 6.86 (t, J = 55.0 Hz, 1H), 1.53-1.46 (m, 2H), 1.19-1.12 (m, 2H). LCMS (Analytical Method F): Rt = 3.30 mins; MS (ESIpos) m/z = 469 (M + H)⁺. |
| 207 | 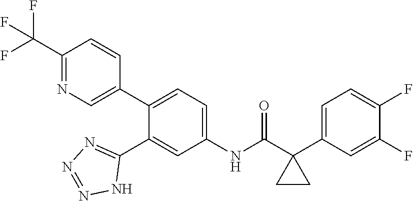 [SMILES: O=C(Nc1ccc(-c2ccc(C(F)(F)F)nc2)c(-c2nnn[nH]2)c1)C1(c2ccc(F)c(F)c2)CC1] | N-{4-[6- (difluoromethyl) pyridin-3-yl]-3- (1H-tetrazol-5- yl)phenyl}-1- (3,4- difluorophenyl) cyclopropane- carboxamide | ¹H NMR (500 MHz, DMSO-d6) δ [ppm] 9.37 (s, 1H), 8.40 (d, J = 1.4 Hz, 1H), 8.06-8.01 (m, 1H), 7.94 (dd, J = 8.5, 2.2 Hz, 1H), 7.66 (dd, J = 8.1, 2.0 Hz, 1H), 7.63 (d, J = 8.0 Hz, 1H), 7.57 (d, J = 8.5 Hz, 1H), 7.50 (ddd, J = 11.8, 7.8, 2.2 Hz, 1H), 7.46- 7.38 (m, 1H), 7.31-7.25 (m, 1H), 6.96 (t, J = 55.0 Hz, 1H), 1.57-1.47 (m, 2H), 1.25-1.15 (m, 2H). LCMS (Analytical Method F): Rt = 3.32 mins; MS (ESIpos) m/z = 469.1 (M + H)⁺ |
| 208 | 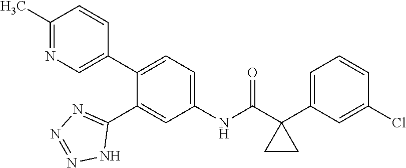 [SMILES: Cc1ccc(-c2ccc(NC(=O)C3(c4cccc(Cl)c4)CC3)cc2-c2nnn[nH]2)cn1] | 1(3- chlorophenyl)- N-[4-(6- methylpyridin- 3-yl)-3-(1H- tetrazol-5- yl)phenyl] cyclopropane- carboxamide | ¹H NMR (500 MHz, DMSO-d6) δ [ppm] 9.55 (s, 1H), 8.19 (d, J = 1.9 Hz, 1H), 8.03-7.95 (m, 1H), 7.91 (dt, J = 8.5, 2.8 Hz, 1H), 7.51 (d, J = 8.5 Hz, 1H), 7.45 (d, J = 1.7 Hz, 1H), 7.42-7.31 (m, 4H), 7.22 (d, J = 8.0 Hz, 1H), 2.46 (s, 3H), 1.53-1.47 (m, 2H), 1.23-1.17 (m, 2H) LCMS (Analytical Method F): Rₜ = 2.31 mins; MS (ESIpos) m/z = 431 (M + H)⁺ |
| 209 | 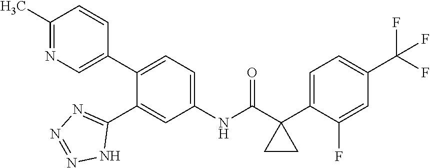 [SMILES: Cc1ccc(-c2ccc(NC(=O)C3(c4ccc(C(F)(F)F)cc4F)CC3)cc2-c2nnn[nH]2)cn1] | 1-[2-fluoro-4- (trifluoromethyl) phenyl]-N-[4- (6- methylpyridin- 3-yl)-3-(1H- tetrazol-5- yl)phenyl] cyclopropane- carboxamide | ¹H NMR (500 MHz, DMSO-d6) δ [ppm] 9.28 (s, 1H), 8.18-8.11 (m, 1H), 7.92-7.83 (m, 2H), 7.75-7.64 (m, 2H), 7.64-7.54 (m, 1H), 7.49 (d, J = 8.4 Hz, 1H), 7.36-7.28 (m, 1H), 7.17 (d, J = 8.0 Hz, 1H), 2.44 (s, 3H), 1.68- 1.62 (m, 2H), 1.29-1.22 (m, 2H). LCMS (Analytical Method F): Rt = 2.52 mins, MS (ESIpos): m/z = 483 (M + H)⁺ |
| 210 | 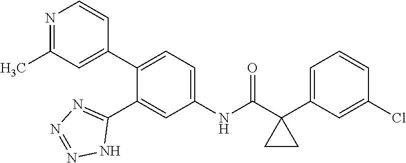 [SMILES: Cc1cc(-c2ccc(NC(=O)C3(c4cccc(Cl)c4)CC3)cc2-c2nnn[nH]2)ccn1] | 1-(3- chlorophenyl)- N-[4-(2- methylpyridin- 4-yl)-3-(1H- tetrazol-5- yl)phenyl] cyclopropane- carboxamide | ¹H NMR (400 MHz, DMSO-d6) δ [ppm] 1.20-1.23 (m, 2H), 1.49- 1.52 (m, 2H), 2.42 (s, 3H), 6.77 (dd, 1H), 7.02 (s, 1H), 7.37-7.40 (m, 3H), 7.45 (dd, 1H), 7.54 (d, 1H), 7.94 (dd, 1H), 7.99 (d, 1H), 8.32 (d, 1H), 9.59 (s, 1H). LCMS (method 1): Rt = 0.84 min, m/z = 431 (M + H)⁺ |
| 211 | 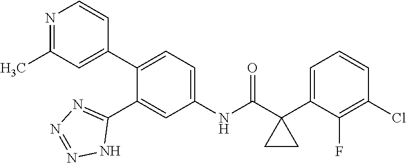 [SMILES: Cc1cc(-c2ccc(NC(=O)C3(c4cccc(Cl)c4F)CC3)cc2-c2nnn[nH]2)ccn1] | 1-(3-chloro-2- fluorophenyl)- N-[4-(2- methylpyridin- 4-yl)-3-(1H- tetrazol-5- yl)phenyl] cyclopropane- carboxamide | ¹H NMR (500 MHz, DMSO-d6) δ [ppm] 9.24 (s, 1H), 8.30 (d, J = 5.1 Hz, 1H), 7.95-7.86 (m, 2H), 7.60-7.54 (m, 1H), 7.51 (d, J = 8.4 Hz, 1H), 7.49-7.41 (m, 1H), 7.28-7.20 (m, 1H), 7.01 (s, 1H), 6.76 (d, J = 4.7 Hz, 1H), 2.41 (s, 3H), 1.70-1.58 (m, 2H), 1.29- 1.17 (m, 2H). LCMS (Analytical Method E): Rt = 3.15 mins; (ESIpos) m/z = 449 (M + H)⁺. |
| 212 | 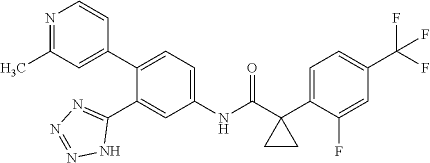 [SMILES: Cc1cc(-c2ccc(NC(=O)C3(c4ccc(C(F)(F)F)cc4F)CC3)cc2-c2nnn[nH]2)ccn1] | 1-[2-fluoro-4- (trifluoromethyl) phenyl]-N-[4- (2- methylpyridin- 4-yl)-3-(1H- tetrazol-5- yl)phenyl] cyclopropane carboxamide | ¹H NMR (500 MHz, DMSO-d6) δ [ppm] 9.29 (s, 1H), 8.29 (d, J = 5.2 Hz, 1H), 7.93-7.86 (m, 2H), 7.75-7.66 (m, 2H), 7.64-7.57 (m, 1H), 7.55-7.45 (m, 1H), 7.00 (s, 1H), 6.79-6.71 (m, 1H), 2.40 (s, 3H), 1.68-1.63 (m, 2H), 1.28-1.22 (m, 2H). LCMS (Analytical Method F): Rt = 2.46 mins, MS (ESIpos): m/z = 483 (M + H)+ |
| 213 | 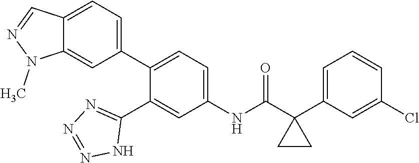 [SMILES: Cn1ncc2ccc(-c3ccc(NC(=O)C4(c5cccc(Cl)c5)CC4)cc3-c3nnn[nH]3)cc21] | 1-(3- chlorophenyl)- N-[4-(1-methyl- 1H-indazol-6- yl)-3-(1H- tetrazol-5- yl)phenyl] cyclopropane- carboxamide | ¹H NMR (500 MHz, DMSO-d6) δ [ppm] 9.52 (s, 1H), 8.01 (d, J = 0.7 Hz, 1H), 7.98-7.88 (m, 2H), 7.58 (m, 2H), 7.50-7.43 (m, 2H), 7.43-7.33 (m, 3H), 6.61 (d, J = 8.3 Hz, 1H), 3.99 (s, 3H), 1.54-1.47 (m, 2H), 1.23-1.16 (m, 2H). LCMS (Analytical Method F): Rt = 3.41 mins; MS (ESIpos) m/z = 470 (M + H)⁺. |
| 214 | 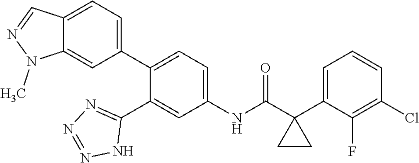 [SMILES: Cn1ncc2ccc(-c3ccc(NC(=O)C4(c5cccc(Cl)c5F)CC4)cc3-c3nnn[nH]3)cc21] | 1-(3-chloro-2- fluorophenyl)- N-[4-(1-methyl- 1H-indazol-6- yl)-3-(1H- tetrazol-5- yl)phenyl] cyclopropane carboxamide | ¹H NMR (500 MHz, DMSO-d6) δ [ppm] 9.18 (s, 1H), 7.99 (d, J = 0.8 Hz, 1H), 7.89-7.82 (m, 2H), 7.61-7.49 (m, 3H), 7.49- 7.42 (m, 2H), 7.28-7.21 (m, 1H), 6.67-6.61 (m, 1H), 3.99 (s, 3H), 1.67-1.60 (m, 2H), 1.27-1.18 (m, 2H). LCMS (Analytical Method F): Rt = 3.35 mins; MS (ESIpos) m/z = 488 (M + H)⁺ |
| 215 | 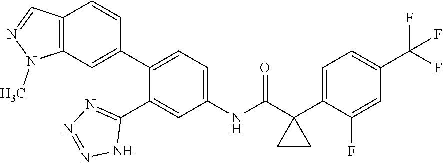 [SMILES: Cn1ncc2ccc(-c3ccc(NC(=O)C4(c5ccc(C(F)(F)F)cc5F)CC4)cc3-c3nnn[nH]3)cc21] | 1-[2-fluoro-4- (trifluoromethyl) phenyl]-N-[4- (1-methyl-1H- indazol-6-yl)-3- (1H-tetrazol-5- yl)phenyl] cyclopropane- carboxamide | ¹H NMR (500 MHz, DMSO-d6) δ [ppm] 9.23 (s, 1H), 7.99 (s, 1H), 7.87-7.81 (m, 2H), 7.75-7.66 (m, 2H), 7.64-7.58 (m, 1H), 7.56-7.49 (m, 2H), 7.46 (s, 1H), 6.67-6.60 (m, 1H), 3.98 (s, 3H), 1.72-1.60 (m, 2H), 1.30-1.18 (m, 2H). LCMS (Analytical Method F): Rt = 3.55 mins, MS (ESIpos): m/z = 522 (M + H)⁺. |
| 216 | 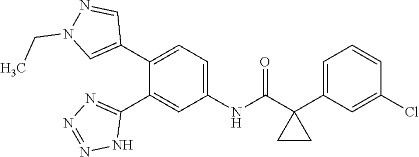 [SMILES: CCn1cc(-c2ccc(NC(=O)C3(c4cccc(Cl)c4)CC3)cc2-c2nnn[nH]2)cn1] | 1-(3- chlorophenyl)- N-[4-(1-ethyl- 1H-pyrazol-4- yl)-3-(1H- tetrazol-5- yl)phenyl] cyclopropane- carboxamide | ¹H NMR (400 MHz, DMSO-d6) δ [ppm] 1.17-1.19 (m, 2H), 1.30 (t, 3H), 1.47-1.50 (m, 2H), 4.05 (q, 2H), 7.35-7.40 (m, 3H), 7.43- 7.44 (m, 1H), 7.49 (d, 1H), 7.55 (d, 1H), 7.74 (d, 1H), 7.82 (dd, 1H), 9.41 (s, 1H). LCMS (method 1): Rt = 1.08 min, m/z = 434 (M + H)⁺ |
| 217 | 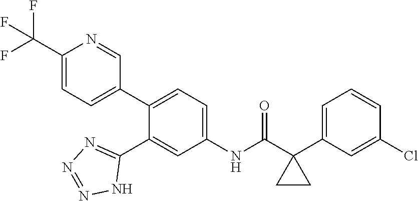 [SMILES: O=C(Nc1ccc(-c2ccc(C(F)(F)F)nc2)c(-c2nnn[nH]2)c1)C1(c2cccc(Cl)c2)CC1] | 1-(3- chlorophenyl)- N-[3-(1H- tetrazol-5-yl)- 6′- (trifluoromethyl)- 2,3′-bipyridin- 5- yl]cyclopropane carboxamide | ¹H NMR (400 MHz, DMSO-d6) δ [ppm] 1.24-1.27 (m, 2H), 1.55- 1.57 (m, 2H), 7.38-7.43 (m, 3H), 7.48 (m, 1H), 7.88 (dd, 1H), 7.93 (dd, 1H), 8.54 (d, 1H), 8.65 (d, 1H), 9.06 (d, 1H), 9.78 (s, 1H). LCMS (method 4): Rt = 0.71 min, m/z = 486 (M + H)+ |
| 218 | 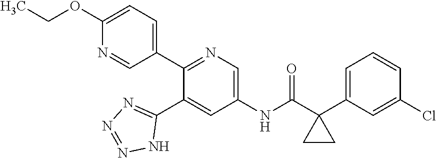 [SMILES: CCOc1ccc(-c2ncc(NC(=O)C3(c4cccc(Cl)c4)CC3)cc2-c2nnn[nH]2)cn1] | 1-(3- chlorophenyl)- N-[6′-ethoxy-3- (1H-tetrazol-5- yl)-2,3′- bipyridin-5- yl]cyclopropane carboxamide | ¹H NMR (400 MHz, DMSO-d6) δ [ppm] 1.21-1.24 (m, 2H), 1.30 (t, 3H), 1.52-1.55 (m, 2H), 4.29 (q, 2H), 6.72 (d, 1H), 7.37-7.40 (m, 3H), 7.46-7.47 (m, 1H), 7.52 (dd, 1H), 7.98 (d, 1H), 8.33 (d, 1H), 8.98 (d, 1H), 9.64 (s, 1H). LCMS (method 3): Rt = 1.15 min, m/z = 462 (M + H)⁺ |
| 219 | 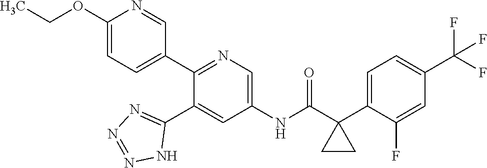 [SMILES: CCOc1ccc(-c2ncc(NC(=O)C3(c4ccc(C(F)(F)F)cc4F)CC3)cc2-c2nnn[nH]2)cn1] | N-[6′-ethoxy-3- (1H-tetrazol-5- yl)-2,3′- bipyridin-5-yl]- 1-[2-fluoro-4- (trifluoromethyl) phenyl] cyclopropane- carboxamide | ¹H NMR (400 MHz, DMSO-d6) δ [ppm] 0.84-0.88 (m, 2H), 1.30 (t, 3H), 1.67-1.70 (m, 2H), 4.29 (q, 2H), 6.71 (d, 1H), 7.49-7.74 (m, 4H), 7.99 (d, 1H), 8.25 (d, 1H), 8.95 (d, 1H), 9.41 (s, 1H). LCMS (method 3): Rt = 1.20 min, m/z = 514 (M + H)⁺ |
| 220 | 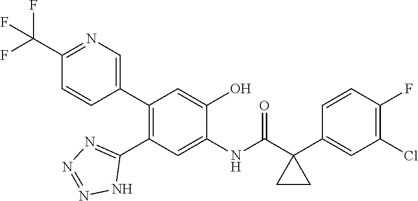 [SMILES: O=C(Nc1cc(-c2nnn[nH]2)c(-c2ccc(C(F)(F)F)nc2)cc1O)C1(c2ccc(F)c(Cl)c2)CC1] | 1-(3-chloro-4- fluorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.24-1.27 (m, 2H), 1.53- 1.56 (m, 2H), 6.92 (s, 1H), 7.54 (t, 1H), 7.59-7.63 (m, 1H), 7.72- 7.75 (m, 1H), 7.84-7.87 (m, 2H), 8.07 (s, 1H), 8.47 (d, 1H), 8.49 (s, br, 1H), 11.03 (s, 1H). LCMS (method 1): Rt = 1.17 min; MS (ESIpos) m/z = 519 (M + H)⁺ |
| 221 | 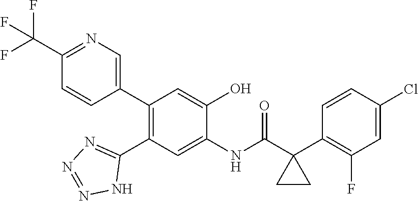 [SMILES: O=C(Nc1cc(-c2nnn[nH]2)c(-c2ccc(C(F)(F)F)nc2)cc1O)C1(c2ccc(Cl)cc2F)CC1] | 1-(4-chloro-2- fluorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.23-1.26 (m, 2H), 1.59- 1.62 (m, 2H), 6.89 (s, 1H), 7.43 (dd, 1H), 7.62 (dd, 1H), 7.64- 7.68 (m, 1H), 7.74 (dd, 1H), 8.06 (s, 1H), 8.45-8.47 (m, 2H), 10.91 (s, 1H). LCMS (method 1): Rt = 1.17 min; MS (ESIpos) m/z = 519 (M + H)⁺ |
| 222 | 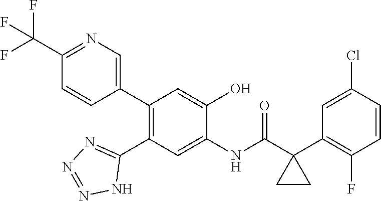 [SMILES: O=C(Nc1cc(-c2nnn[nH]2)c(-c2ccc(C(F)(F)F)nc2)cc1O)C1(c2cc(Cl)ccc2F)CC1] | 1-(5-chloro-2- fluorophenyl)- N-{2-hydroxy-5- (1H-tetrazol-5- yl)-4-[6- (trifluoromethyl- pyridin-3- yl]phenyl} cyclopropane- carboxamide | ¹H NMR (400 MHz, DMSO-d6) δ [ppm] 1.27-1.30 (m, 2H), 1.58- 1.61 (m, 2H), 6.92 (s, 1H), 7.41 (dd, 1H), 7.56-7.60 (m, 1H), 7.71-7.76 (m, 2H), 7.85 (d, 1H), 8.14 (s, 1H), 8.46-8.47 (m, 2H), 11.0 (s, 1H). LCMS (method 1): Rₜ = 1.16 min; MS (ESIpos) m/z = 519 (M + H)⁺ |
| 223 | 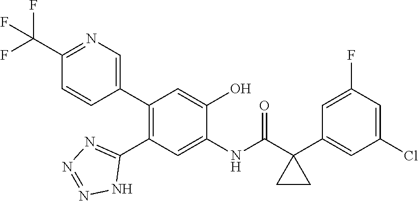 [SMILES: O=C(Nc1cc(-c2nnn[nH]2)c(-c2ccc(C(F)(F)F)nc2)cc1O)C1(c2cc(F)cc(Cl)c2)CC1] | 1-(3-chloro-5- fluorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.27-1.30 (m, 2H), 1.53- 1.56 (m, 2H), 6.94 (s, 1H), 7.47- 7.55 (m, 3H), 7.75 (dd, 1H), 7.85 (d, 1H), 8.18 (s, 1H), 8.47 (d, 1H), 11.03 (s, 1H). LCMS (method 1): Rₜ = 1.18 min; MS (ESIpos) m/z = 519 (M + H)⁺ |
| 224 | 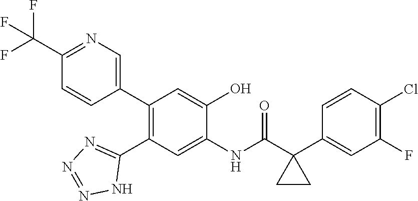 [SMILES: O=C(Nc1cc(-c2nnn[nH]2)c(-c2ccc(C(F)(F)F)nc2)cc1O)C1(c2ccc(Cl)c(F)c2)CC1] | 1-(4-chloro-3- fluorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.24-1.27 (m, 2H), 1.55 1.57 (m, 2H), 6.89 (s, 1H), 7.46 (dd, 1H), 7.67-7.75 (m, 3H), 7.83 (d, 1H), 8.11 (s, 1H), 8.45- 8.46 (m, 2H), 10.86 (s, 1H). LCMS (method 1): Rₜ = 1.18 min; MS (ESIpos) m/z = 519 (M + H)⁺ |
| 225 | 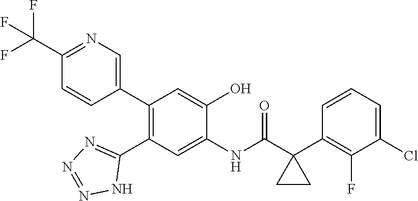 [SMILES: O=C(Nc1cc(-c2nnn[nH]2)c(-c2ccc(C(F)(F)F)nc2)cc1O)C1(c2cccc(Cl)c2F)CC1] | 1-(3-chloro-2- fluorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.27-1.30 (m, 2H), 1.62- 1.65 (m, 2H), 6.91 (s, 1H), 7.34- 7.37 (m, 1H), 7.59-7.63 (m, 1H), 7.67-7.71 (m, 1H), 7.73- 7.75 (m, 1H), 7.83 (d, 1H), 8.09 (s, 1H), 8.46-8.47 (m, 2H), 10.91 (s, 1H). LCMS (method 1): Rₜ = 1.15 min; MS (ESIpos) m/z = 519 (M + H)⁺ |
| 226 | 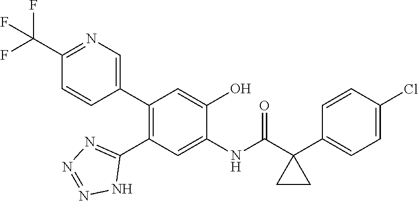 [SMILES: O=C(Nc1cc(-c2nnn[nH]2)c(-c2ccc(C(F)(F)F)nc2)cc1O)C1(c2ccc(Cl)cc2)CC1] | 1-(4- chlorophenyl)- N-{2-hydroxy-5- (1H-tetrazol-5- yl)-4-[6- (trifluoromethyl) pyridin-3- yl]phenyl} cyclopropane- carboxamide | ¹H NMR (400 MHz, DMSO-d6) δ [ppm] 1.20-1.22 (m, 2H), 1.55- 1.57 (m, 2H), 6.86 (s, 1H), 7.55 (d, 2H), 7.61 (d, 2H), 7.72 (dd, 1H), 7.82 (d, 1H), 8.01 (s, 1H), 8.43 (d, 1H), 8.55 (s, 1H), 10.86 (s, 1H). LCMS (method 1): Rₜ = 1.16 min; MS (ESIpos) m/z = 501 (M + H)⁺ |
| 227 | 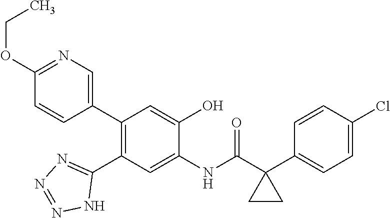 [SMILES: CCOc1ccc(-c2cc(O)c(NC(=O)C3(c4ccc(Cl)cc4)CC3)cc2-c2nnn[nH]2)cn1] | 1-(4- chlorophenyl)- N-[4-(6- ethoxypyridin-3- yl)-2-hydroxy-5- (1H-tetrazol-5- yl)phenyl] cyclopropane- carboxamide | ¹H NMR (400 MHz, DMSO-d6) δ [ppm] 1.18-1.21 (m, 2H), 1.30 (t, 3H), 1.53-1.55 (m, 2H), 4.27 (q, 2H), 6.68 (d, 1H), 6.81 (s, 1H), 7.26 (dd, 1H), 7.54 (d, 2H), 7.60 (d, 2H), 7.82 (d, 1H), 7.96 (s, 1H), 8.41 (s, 1H), 10.80 (s, 1H). LCMS (method 1): Rₜ = 1.13 min; MS (ESIpos) m/z = 477 (M + H)⁺ |
| 228 | 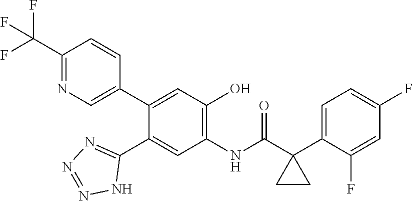 [SMILES: O=C(Nc1cc(-c2nnn[nH]2)c(-c2ccc(C(F)(F)F)nc2)cc1O)C1(c2ccc(F)cc2F)CC1] | 1-(2,4- Difluorophenyl)- N-{2-hydroxy-5- (1H-tetrazol-5- yl)-4-[6- (trifluoromethyl) pyridin-3- yl]phenyl} cyclopropane- carboxamide | ¹H NMR (500 MHz, DMSO-d6) δ [ppm] 11.03 (s, 1H), 8.61-8.36 (m, 2H), 8.06 (s, 1H), 7.84 (d, J = 8.1 Hz, 1H), 7.76-7.72 (m, 1H), 7.71-7.65 (m, 1H), 7.46-7.36 (m, 1H), 7.26-7.18 (m, 1H), 6.92 (s, 1H), 1.68-1.51 (m, 2H), 1.30-1.14 (m, 2H). LCMS (Analytical Method D): Rt = 4.13 mins; MS (ESIpos) m/z = 503.0 (M + H)⁺. |
| 229 | 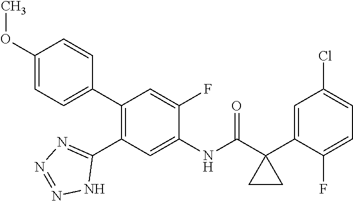 [SMILES: COc1ccc(-c2cc(F)c(NC(=O)C3(c4cc(Cl)ccc4F)CC3)cc2-c2nnn[nH]2)cc1] | 1-(5-Chloro-2- fluorophenyl)- N-[5-fluoro-4′- methoxy-2-(1H- tetrazol-5- yl)biphenyl-4- yl]cyclopropane carboxamide | ¹H NMR (400 MHz, DMSO-d6) δ [ppm] 1.22-1.25 (m, 2H), 1.55- 1.57 (m, 2H), 3.74 (s, 3H), 6.86 (d, 2H), 7.01 (d, 2H), 7.30 (dd, 1H), 7.39 (d, 1H), 7.45-7.49 (m, 1H), 7.56 (dd, 1H), 7.63 (d, 1H), 8.95 (s, 1H). LCMS (method 2): Rt = 1.24 min; MS (ESIpos) m/z = 482 (M + H)⁺. |
| 230 | 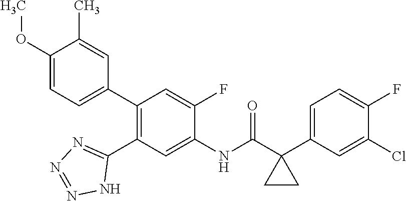 [SMILES: COc1ccc(-c2cc(F)c(NC(=O)C3(c4ccc(F)c(Cl)c4)CC3)cc2-c2nnn[nH]2)cc1C] | 1-(3-Chloro-4- fluorophenyl)- N-[5-fluoro-4′- methoxy-3′- methyl-2-(1H- tetrazol-5- yl)biphenyl-4- yl]cyclopropane- carboxamide | ¹H NMR (400 MHz, DMSO-d6) δ [ppm] 1.19-1.21 (m, 2H), 1.48- 1.51 (m, 2H), 2.07 (s, 3H), 3.76 (s, 3H), 6.77-6.84 (m, 2H), 6.95 (d, 1H), 7.35-7.51 (m, 3H), 7.87- 7.70 (m, 2H), 8.84 (s, 1H). LCMS (method 1): Rt = 1.36 min; MS (ESIpos) m/z = 496 (M + H)⁺. |
| 231 | 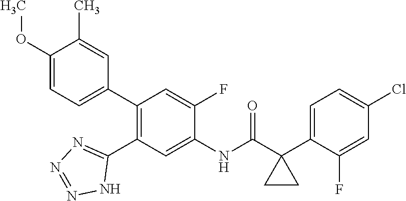 [SMILES: COc1ccc(-c2cc(F)c(NC(=O)C3(c4ccc(Cl)cc4F)CC3)cc2-c2nnn[nH]2)cc1C] | 1-(4-Chloro-2- fluorophenyl)- N-[5-fluoro-4′- methoxy-3′- methyl-2-(1H- tetrazol-5- yl)biphenyl-4- yl]cyclopropane carboxamide | ¹H NMR (400 MHz, DMSO-d6) δ [ppm] 1.16-1.19 (m, 2H), 1.51- 1.55 (m, 2H), 2.08 (s, 3H), 3.76 (s, 3H), 6.78 (dd, 1H), 6.83 (d, 1H), 6.95 (d, 1H), 7.31-7.43 (m, 2H), 7.47-7.59 (m, 3H), 8.89 (s, 1H). LCMS (method 1): Rt = 1.35 min; MS (ESIpos) m/z = 496 (M + H)⁺. |
| 232 | 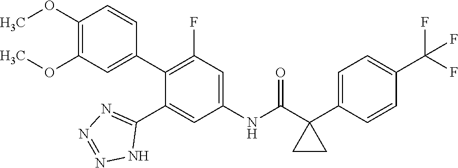 [SMILES: COc1ccc(-c2c(F)cc(NC(=O)C3(c4ccc(C(F)(F)F)cc4)CC3)cc2-c2nnn[nH]2)cc1OC] | N-[2-Fluoro- 3′,4′-dimethoxy- 6-(1H-tetrazol- 5-yl)biphenyl-4- yl]-1-[4- (trifluoromethyl) phenyl] cyclopropane- carboxamide | ¹H NMR (500 MHz, DMSO-d6) δ [ppm] 9.71 (s, 1H), 7.88 (dd, J = 12.3, 2.1 Hz, 1H), 7.75-7.68 (m, 3H), 7.60 (d, J = 8.1 Hz, 2H), 6.88 (d, J = 8.9 Hz, 1H), 6.63- 6.56 (m, 2H), 3.73 (s, 3H), 3.59 (s, 3H), 1.64-1.51 (m, 2H), 1.31- 1.20 (m, 2H). LCMS (Analytical Method D): Rt = 4.47 mins; MS (ESIpos) m/z = 528.0 (M + H)⁺. |
| 233 | 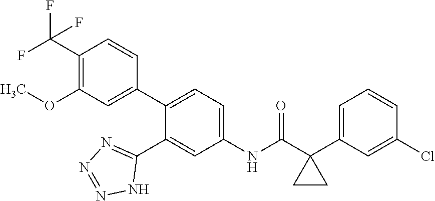 [SMILES: COc1cc(-c2ccc(NC(=O)C3(c4cccc(Cl)c4)CC3)cc2-c2nnn[nH]2)ccc1C(F)(F)F] | 1-(3- chlorophenyl)- N-[3′-methoxy- 2-(1H-tetrazol- 5-yl)-4′- (trifluoromethyl) biphenyl-4- yl]cyclopropane carboxamide | ¹H NMR (500 MHz, DMSO-d6) δ [ppm] 1.20-1.23 (m, 2H), 1.50- 1.52 (m, 2H), 3.75 (s, 3H), 6.71 (d, 1H), 6.91 (s, 1H), 7.36-7.40 (m, 3H), 7.45-7.46 (m, 1H), 7.50 (d, 1H), 7.59 (d, 1H), 7.93 (dd, 1H), 7.97 (d, 1H), 9.58 (s, 1H). LCMS (method 4): Rt = 0.86 min, m/z = 514 (M + H)⁺ |
| 234 | 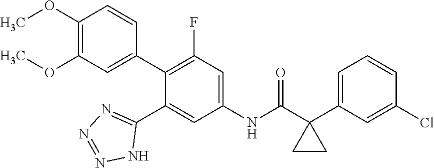 [SMILES: COc1ccc(-c2c(F)cc(NC(=O)C3(c4cccc(Cl)c4)CC3)cc2-c2nnn[nH]2)cc1OC] | 1-(3- Chlorophenyl)- N-[2-fluoro- 3′,4′-dimethoxy- 6-(1H-tetrazol- 5-yl)biphenyl-4- yl]cyclopropane carboxamide | ¹H NMR (500 MHz, DMSO-d6) δ [ppm] 9.62 (s, 1H), 7.87 (dd, J = 12.3, 1.8 Hz, 1H), 7.71 (s, 1H), 7.44 (s, 1H), 7.42-7.34 (m, 3H), 6.92-6.85 (m, 1H), 6.62-6.56 (m, 2H), 3.73 (s, 3H), 3.58 (s, 3H), 1.60-1.41 (m, 2H), 1.32- 1.08 (m, 2H). LCMS (Analytical Method D): Rt = 4.38 mins; MS (ESIpos) m/z = 494.1 (M + H)⁺. |
| 235 | 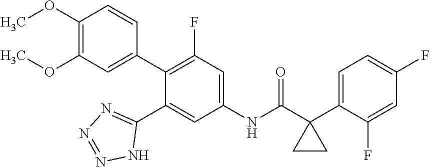 [SMILES: COc1ccc(-c2c(F)cc(NC(=O)C3(c4ccc(F)cc4F)CC3)cc2-c2nnn[nH]2)cc1OC] | 1-(2,4- Difluorophenyl)- N-[2-fluoro- 3′,4′-dimethoxy- 6-(1H-tetrazol- 5-yl)biphenyl-4- yl]cyclopropane carboxamide | ¹H NMR (500 MHz, DMSO-d6) δ [ppm] 9.19 (s, 1H), 7.81 (dd, J = 12.4, 2.0 Hz, 1H), 7.64 (d, J = 1.6 Hz, 1H), 7.57-7.47 (m, 1H), 7.29-7.21 (m, 1H), 7.13-7.05 (m, 1H), 6.86 (d, J = 8.1 Hz, 1H), 6.64-6.56 (m, 2H), 3.73 (s, 3H), 3.57 (s, 3H), 1.63-1.57 (m, 2H), 1.20-1.14 (m, 2H). LCMS (Analytical Method D): Rt = 4.33 mins; MS (ESIpos) m/z = 496.0 (M + H)⁺. |
| 236 | 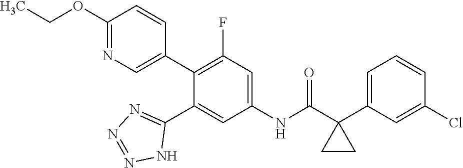 [SMILES: CCOc1ccc(-c2c(F)cc(NC(=O)C3(c4cccc(Cl)c4)CC3)cc2-c2nnn[nH]2)cn1] | 1-(3- chlorophenyl)- N-[4-(6- ethoxypyridin-3- yl)-3-fluoro-5- (1H-tetrazol-5- yl)phenyl] cyclopropane- carboxamide | ¹H NMR (500 MHz, DMSO-d6) δ [ppm] 9.70 (s, 1H), 7.94-7.77 (m, 3H), 7.48-7.32 (m, 5H), 6.75 (d, J = 8.6 Hz, 1H), 4.28 (q, J = 7.0 Hz, 2H), 1.55-1.46 (m, 2H), 1.30 (t, J = 7.0 Hz, 3H), 1.26-1.18 (m, 2H) LCMS (Analytical Method F): Rt = 3.77 mins, MS (ESIpos): m/z = 479 (M + H)⁺. |
| 240 | 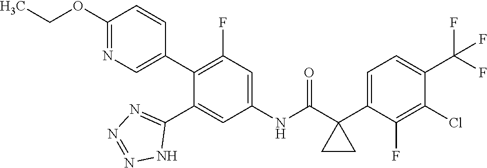 | N-[4-(6- ethoxypyridin-3- yl)-3-fluoro-5- (1H-tetrazol-5- yl)phenyl]-1-[2- fluoro-4- (trifluoromethyl) phenyl] cyclopropane- carboxamide | ¹H NMR (500 MHz, Methanol-d4) δ [ppm] 7.79 (s, 1H), 7.76-7.65 (m, 2H), 7.59-7.46 (m, 3H), 7.40-7.35 (m, 1H), 6.68 (d, J = 8.6 Hz, 1H), 4.26 (q, J = 7.1 Hz, 2H), 1.79-1.69 (m, 2H), 1.35 (t, J = 7.1 Hz, 3H), 1.32-1.23 (m, 2H). LCMS (Analytical Method F): Rt = 3.88 mins, MS (ESIpos): m/z = 531 (M + H)⁺. |
| 241 | 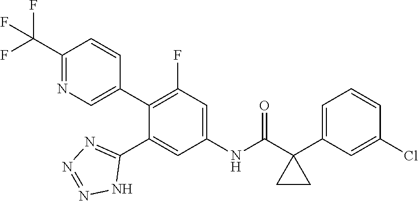 [SMILES: O=C(Nc1cc(F)c(-c2ccc(C(F)(F)F)nc2)c(-c2nnn[nH]2)c1)C1(c2cccc(Cl)c2)CC1] | 1-(3- chlorophenyl)- N-{3-fluoro-5- (1H-tetrazol-5- yl)-4-[6- (trifluoromethyl) pyridin-3- yl]phenyl} cyclopropane- carboxamide | ¹H NMR (500 MHz, DMSO-d6) δ [ppm] 9.78 (s, 1H), 8.55 (s, 1H), 8.00 (s, 1H), 7.95 (dd, J = 12.3, 1.9 Hz, 1H), 7.91 (d, J = 8.1 Hz, 1H), 7.90-7.86 (m, 1H), 7.48- 7.43 (m, 1H), 7.43-7.35 (m, 3H), 1.59-1.47 (m, 2H), 1.29- 1.18 (m, 2H). LCMS (Analytical Method F): Rt = 3.85 mins; MS (ESIpos) m/z = 503.1 (M + H)⁺. |
| 242 | 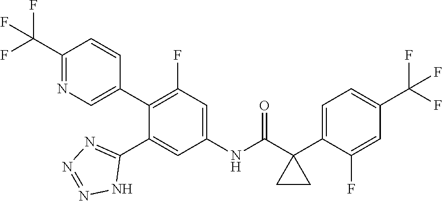 [SMILES: O=C(Nc1cc(F)c(-c2ccc(C(F)(F)F)nc2)c(-c2nnn[nH]2)c1)C1(c2ccc(C(F)(F)F)cc2F)CC1] | N-{3-fluoro-5- (1H-tetrazol-5- yl)-4-[6- (trifluoromethyl) pyridin-3- yl]phenyl}-1-[2- fluoro-4- (trifluoromethyl) phenyl] cyclopropane- carboxamide | ¹H NMR (500 MHz, DMSO-d6) δ [ppm] 9.51 (s, 1H), 8.56 (s, 1H), 7.99 (s, 1H), 7.95-7.86 (m, 3H), 7.76-7.67 (m, 2H), 7.63 (d, J = 8.0 Hz, 1H), 1.75-1.63 (m, 2H), 1.37-1.25 (m, 2H). LCMS (Analytical Method F): Rt = 3.95 mins; MS (ESIpos) m/z = 555.1 (M + H)⁺. |
| 243 | 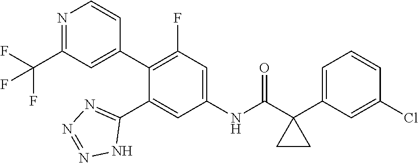 [SMILES: O=C(Nc1cc(F)c(-c2ccnc(C(F)(F)F)c2)c(-c2nnn[nH]2)c1)C1(c2cccc(Cl)c2)CC1] | 1-(3- chlorophenyl)- N-{3-fluoro-5- (1H-tetrazol-5- yl)-4-[2- (trifluoromethyl) pyridin-4- yl]phenyl} cyclopropane- carboxamide | ¹H NMR (500 MHz, DMSO-d6) δ [ppm] 9.78 (s, 1H), 8.73 (d, J = 5.0 Hz, 1H), 7.99 (s, 1H), 7.95 (dd, J = 12.4, 2.0 Hz, 1H), 7.70 (s, 1H), 7.48 (d, J = 4.9 Hz, 1H), 7.46-7.44 (m, 1H), 7.43-7.35 (m, 3H), 1.58-1.48 (m, 2H), 1.29-1.18 (m, 2H). LCMS (Analytical Method F): Rt = 3.86 mins; MS (ESIpos) m/z = 503.0 (M + H)⁺. |
| 244 | 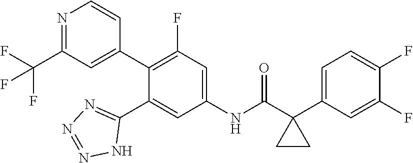 [SMILES: O=C(Nc1cc(F)c(-c2ccnc(C(F)(F)F)c2)c(-c2nnn[nH]2)c1)C1(c2ccc(F)c(F)c2)CC1] | 1-(3,4- difluorophenyl)- N-{3-fluoro-5- (1H-tetrazol-5- yl)-4-[2- (trifluoromethyl) pyridin-4- yl]phenyl} cyclopropane- carboxamide | ¹H NMR (500 MHz, DMSO-d6) δ [ppm] 9.55 (s, 1H), 8.73 (d, J = 5.0 Hz, 1H), 7.98 (s, 1H), 7.95 (dd, J = 12.4, 1.9 Hz, 1H), 7.70 (s, 1H), 7.51 (ddd, J = 11.8, 7.8, 2.2 Hz, 1H), 7.48 (d, J = 4.9 Hz, 1H), 7.46-7.39 (m, 1H), 7.32- 7.24 (m, 1H), 1.58-1.48 (m, 2H), 1.28-1.18 (m, 2H). LCMS (Analytical Method F): Rt = 3.73 mins; MS (ESIpos) m/z = 505.1 (M + H)⁺. |
| 245 | 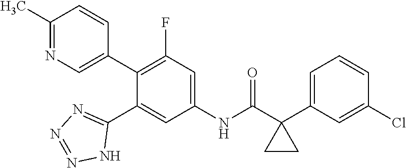 [SMILES: Cc1ccc(-c2c(F)cc(NC(=O)C3(c4cccc(Cl)c4)CC3)cc2-c2nnn[nH]2)cn1] | 1-(3- chlorophenyl)- N-[3-fluoro-4- (6- methylpyridin- 3-yl)-5-(1H- tetrazol-5- yl)phenyl] cyclopropane- carboxamide | ¹H NMR (500 MHz, DMSO-d6) δ [ppm] 9.67 (s, 1H), 8.14 (s, 1H), 7.90-7.77 (m, 2H), 7.44 (d, J = 1.7 Hz, 1H), 7.43-7.34 (m, 4H), 7.20 (d, J = 8.0 Hz, 1H), 2.46 (s, 3H), 1.56-1.44 (m, 2H), 1.28- 1.16 (m, 2H). LCMS (Analytical Method F): Rt = 2.60 mins; MS (ESIpos) m/z = 449 (M + H)⁺. |
| 246 | 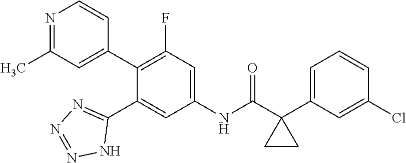 [SMILES: Cc1cc(-c2c(F)cc(NC(=O)C3(c4cccc(Cl)c4)CC3)cc2-c2nnn[nH]2)ccn1] | 1-(3- chlorophenyl)- N-[3-fluoro-4- (2- methylpyridin- 4-yl)-5-(1H- tetrazol-5- yl)phenyl] cyclopropane carboxamide | ¹H NMR (500 MHz, DMSO-d6) δ [ppm] 9.58 (s, 1H), 8.27 (d, J = 5.1 Hz, 1H), 7.76-7.65 (m, 2H), 7.44 (d, J = 1.7 Hz, 1H), 7.42- 7.27 (m, 3H), 6.96 (s, 1H), 6.82 (d, J = 5.0 Hz, 1H), 2.39 (s, 3H), 1.53-1.45 (m, 2H), 1.22-1.13 (m, 2H). LCMS (Analytical Method F): Rt = 2.52 mins; MS (ESIpos) m/z = 449 (M + H)⁺. |
| 247 | 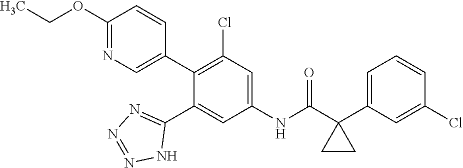 | N-[3-chloro-4- (6- ethoxypyridin-3- yl)-5-(1H- tetrazol-5- yl)phenyl]-1-(3- chlorophenyl) cyclopropane- carboxamide | ¹H NMR (250 MHz, DMSO-d6) δ [ppm] 1.16-1.26 (m, 2H), 1.30 (t, J = 7.0 Hz, 3H), 1.45-1.58 (m, 2H), 4.27 (q, J = 7.0 Hz, 2H), 6.74 (d, J = 8.5 Hz, 1H), 7.35- 7.48 (m, 5H), 7.82 (d, J = 1.9 Hz, 1H), 7.97 (d, J = 2.1 Hz, 1H), 8.10 (d, J = 2.1 Hz, 1H), 9.61 (s, 1H). LCMS (Analytical Method D): Rt = 4.48 mins; MS (ESIpos) m/z = 282.9 (M + H)⁺. |
| 248 | 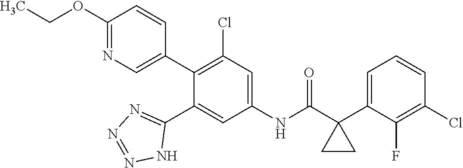 [SMILES: CCOc1ccc(-c2c(Cl)cc(NC(=O)C3(c4cccc(Cl)c4F)CC3)cc2-c2nnn[nH]2)cn1] | N-[3-chloro-4- (6- ethoxypyridin-3- yl)-5-(1H- tetrazol-5- yl)phenyl]-1-(3- chloro-2- fluoropheny) cyclopropane- carboxamide | ¹H NMR (250 MHz, DMSO-d6) δ [ppm] 1.20-1.26 (m, 2H), 1.30 (t, J = 7.1 Hz, 3H), 1.57-1.71 (m, 2H), 4.27 (q, J = 7.0 Hz, 2H), 6.74 (d, J = 8.5 Hz, 1H), 7.17- 7.33 (m, 1H), 7.34-7.50 (m, 2H), 7.50-7.64 (m, 1H), 7.82 (d, J = 2.0 Hz, 1H), 7.97 (s, 1H), 8.08 (d, J = 2.1 Hz, 1H), 9.33 (s, 1H). LCMS (Analytical Method F): Rt = 4.61 mins; MS (ESIpos) m/z = 513.0 (M + H)⁺. |
| 249 | 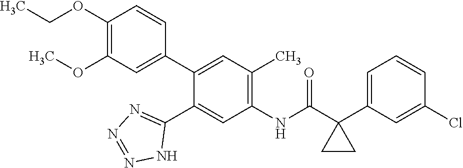 [SMILES: CCOc1ccc(-c2cc(C)c(NC(=O)C3(c4cccc(Cl)c4)CC3)cc2-c2nnn[nH]2)cc1OC] | 1-(3- Chlorophenyl)- N-[4′-ethoxy-3′- methoxy-5- methyl-2-(1H- tetrazol-5- yl)biphenyl-4- yl]cyclopropane carboxamide | ¹H NMR (500 MHz, DMSO-d6) δ [ppm] 1.15-1.23 (m, 2H), 1.30 (t, J = 7.0 Hz, 3H), 1.43-1.54 (m, 2H), 2.14 (s, 3H), 3.58 (s, 3H), 3.98 (q, J = 7.0 Hz, 2H), 6.52-6.61 (m, 2H), 6.85 (d, J = 8.3 Hz, 1H), 7.37-7.49 (m, 4H), 7.54 (s, 1H), 7.58 (s, 1H), 8.57 (s, 1H). LCMS (Analytical Method F): Rt = 3.78 mins; MS (ESIpos) m/z = 504 (M + H)⁺. |
| 250 | 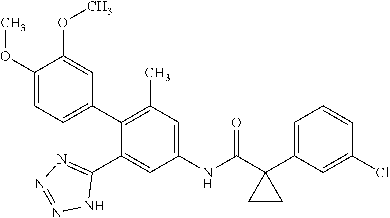 [SMILES: COc1ccc(-c2c(C)cc(NC(=O)C3(c4cccc(Cl)c4)CC3)cc2-c2nnn[nH]2)cc1OC] | 1-(3- Chlorophenyl)- N-[3′,4′- dimethoxy-2- methyl-6-(1H- tetrazol-5- yl)biphenyl-4- yl]cyclopropane carboxamide | ¹H NMR (500 MHz, DMSO-d6) δ [ppm] 9.36 (s, 1H), 7.74-7.66 (m, 2H), 7.45-7.42 (m, 1H), 7.42-7.33 (m, 3H), 6.83 (d, J = 8.3 Hz, 1H), 6.57 (d, J = 1.9 Hz, 1H), 6.47 (dd, J = 8.2, 1.9 Hz, 1H), 3.72 (s, 3H), 3.63 (s, 3H), 2.12 (s, 3H), 1.53-1.44 (m, 2H), 1.24-1.13 (m, 2H). LCMS (Analytical Method D): Rt = 4.40 mins; MS (ESIpos) m/z = 490.15 (M + H)⁺. |
| 251 | 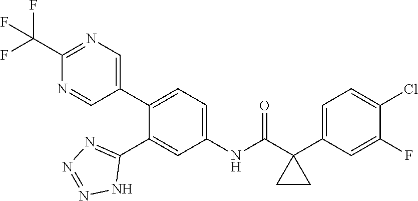 [SMILES: O=C(Nc1ccc(-c2cnc(C(F)(F)F)nc2)c(-c2nnn[nH]2)c1)C1(c2ccc(Cl)c(F)c2)CC1] | 1-(4-chloro-3- fluorophenyl)- N-{3-(1H- tetrazol-5-yl)-4- [2- (trifluoromethyl) pyrimidin-5- yl]phenyl} cyclopropane- carboxamide | ¹H NMR (250 MHz, DMSO-d6) δ [ppm] 1.20-1.28 (m, 2H), 1.48- 1.59 (m, 2H), 7.29 (dd, J = 8.3, 1.5 Hz, 1H), 7.47 (dd, J = 10.6, 2.1 Hz, 1H), 7.51-7.65 (m, 2H), 7.95 (dd, J = 8.5, 2.2 Hz, 1H), 8.20 (d, J = 2.1 Hz, 1H), 8.86 (s, 2H), 9.52 (s, 1H). LCMS (Analytical Method F): Rt = 3.72 mins; m/z (ESI) = 504.0 (M + H)⁺. |
| 252 | 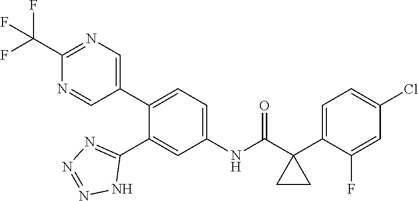 [SMILES: O=C(Nc1ccc(-c2cnc(C(F)(F)F)nc2)c(-c2nnn[nH]2)c1)C1(c2ccc(Cl)cc2F)CC1] | 1-(4-chloro-2- fluorophenyl)- N-{3-{1H- tetrazol-5-yl)-4- [2- (trifluoromethyl) pyrimidin-5- yl]phenyl} cyclopropane- carboxamide | ¹H NMR (250 MHz, DMSO-d6) δ [ppm] 9.32 (s, 1H), 8.88 (s, 2H), 8.19 (d, J = 2.0 Hz, 1H), 7.95 (dd, J = 8.6, 2.2 Hz, 1H), 7.63 (d, J = 8.5 Hz, 1H), 7.58-7.41 (m, 2H), 7.33 (dd, J = 8.2, 1.8 Hz, 1H), 1.69-1.57 (m, 2H), 1.28-1.12 (m, 2H). LCMS (Analytical Method D): Rt = 3.68 mins; m/z (ESI) = 504.1 (M + H)⁺. |

### Example 253: 1-(3-Chlorophenyl)-N-[4-(6-ethoxypyridin-3-yl)-3-(1H-tetrazol-5-yl) phenyl]cyclopropanecarboxamide

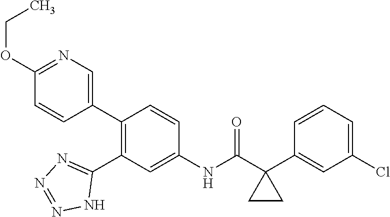 [SMILES: CCOc1ccc(-c2ccc(NC(=O)C3(c4cccc(Cl)c4)CC3)cc2-c2nnn[nH]2)cn1]

a) 1-(3-Chlorophenyl)cyclopropanecarboxylic acid (CAS 124276-34-2) (15 g, 76.3 mmol) and thionyl chloride (90.8 g, 763 mmol) were heated to reflux until gas evaporation ceased (1 h). The mixture was cooled to RT and the excess thionyl chloride was evaporated under reduced pressure. The crude acid chloride was used without further purification.

b) 4-(6-Ethoxypyridin-3-yl)-3-(1H-tetrazol-5-yl)aniline (Intermediate 55A) (720 mg, 2.55 mmol) and triethylamine (310 mg, 3.06 mmol) were dissolved in dichloromethane (16 mL) and cooled to 0° C. 1-(3-Chlorophenyl) cyclopropanecarbonyl chloride (549 mg, 2.55 mmol), which was prepared under a), was added dropwise. After 30 min the cooling bath was removed and the mixture stirred for further 30 min. The mixture was put into water and extracted 3× with dichloromethane. The combined organic layers were washed with brine, dried with sodium sulfate and the solvents removed under reduced pressure. The crude product was purified by flash chromatography (SiO₂, DCM/MeOH 0-5%) to give 712 mg (57% yield) of the title compound as off white solid.

¹H NMR (500 MHz, DMSO-d6) δ[ppm] 9.51 (s, 1H), 7.93 (s, 1H), 7.87 (dd, J=8.4, 2.1 Hz, 2H), 7.47 (d, J=8.5 Hz, 1H), 7.44 (s, 1H), 7.41-7.34 (m, 3H), 7.29 (dd, J=8.6, 2.4 Hz, 1H), 6.69 (d, J=8.6 Hz, 1H), 4.28 (q, J=7.0 Hz, 2H), 1.49 (q, J=4.4 Hz, 2H), 1.30 (t, J=7.0 Hz, 3H), 1.19 (q, J=4.6 Hz, 2H).

LCMS (Analytical Method F): Rt=3.59 min; MS (ESipos) m/z=460.1 (M+H)⁺.

In analogy to Example 253 the following examples were prepared using the corresponding amines and carboxylic acids as starting materials:

| Ex. | Structure | Name | Analytical Data |
|---|---|---|---|
| 254 | 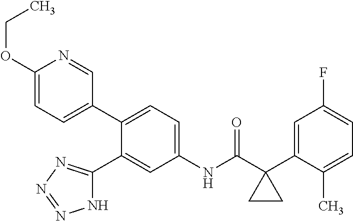 [SMILES: CCOc1ccc(-c2ccc(NC(=O)C3(c4cc(F)ccc4C)CC3)cc2-c2nnn[nH]2)cn1] | N-[4-(6- ethoxypyridin-3- yl)-3-(1H- tetrazol-5- yl)phenyl]-1-(5- fluoro-2- methylphenyl)cy- clopropanecar- boxamide | ¹H NMR (400 MHz, DMSO-d6) δ [ppm] 1.13-1.16 (m, 2H), 1.30 (t, 3H), 1.58-1.61 (m, 2H), 2.29 (s, 3H), 4.28 (q, 2H), 6.70 (d, 1H), 7.09 (td, 1H), 7.22-7.31 (m, 3H), 7.46 (d, 1H), 7.84-7.89 (m, 3H), 8.94 (s, 1H). LCMS (method 1): Rₜ = 1.22 min; MS (ESIpos) m/z = 459 (M + H)⁺. |
| 255 | 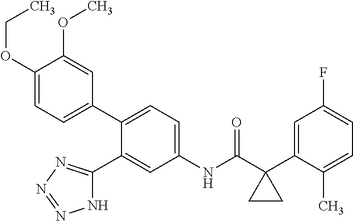 | N-[3′,4′- dimethoxy-2- (1H-tetrazol-5- yl)biphenyl-4- yl]-1-(5-fluoro- 2- methylphenyl)cy- clopropanecar- boxamide | ¹H NMR (400 MHz, DMSO-d6) δ [ppm] 1.13-1.15 (m, 2H), 1.57- 1.60 (m, 2H), 2.28 (s, 3H), 3.57 (s, 3H), 3.73 (s, 3H), 6.55-6.58 (m, 2H), 6.87 (d, 1H), 7.06-7.11 (m, 1H), 7.21-7.32 (m, 2H), 7.48 (d, 1H), 7.76 (d, 1H), 7.84 (dd, 1H), 8.90 (s, 1H). LCMS (method 1): Rₜ = 11.8 min; MS (ESIpos) m/z = 474 (M + H)⁺. |
| 256 | 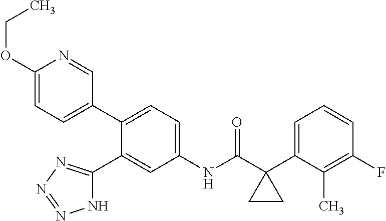 [SMILES: CCOc1ccc(-c2ccc(NC(=O)C3(c4cccc(F)c4C)CC3)cc2-c2nnn[nH]2)cn1] | N-[4-(6- ethoxypyridin-3- yl)-3-(1H- tetrazol-5- yl)phenyl]-1-(3- fluoro-2- methylphenyl)cy- clopropanecar- boxamide | ¹H NMR (400 MHz, DMSO-d6) δ [ppm] 1.13-1.16 (m, 2H), 1.30 (t, 3H), 1.62-1.64 (m, 2H), 2.25 (d, 3H), 4.28 (q, 2H), 6.70 (d, 1H), 7.13-7.18 (m, 1H), 7.26- 7.31 (m, 3H), 7.46 (d, 1H), 7.83- 7.89 (m, 3H), 8.90 (s, 1H). LCMS (method 1): Rₜ = 1.22 min; MS (ESIpos) m/z = 459 (M + H)⁺. |
| 257 | 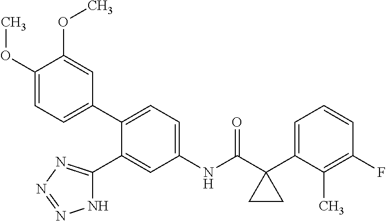 [SMILES: COc1ccc(-c2ccc(NC(=O)C3(c4cccc(F)c4C)CC3)cc2-c2nnn[nH]2)cc1OC] | N-[3′,4′- dimethoxy-2- (1H-tetrazol-5- yl)biphenyl-4- yl]-1-(3-fluoro- 2- methylphenyl)cy- clopropanecar- boxamide | ¹H NMR (400 MHz, DMSO-d6) δ [ppm] 1.13-1.15 (m, 2H), 1.61- 1.64 (m, 2H), 2.25 (d, 3H), 3.57 (s, 3H), 3.73 (s, 3H), 6.55-6.58 (m, 2H), 6.87 (d, 1H), 7.13-7.18 (m, 1H), 7.25-7.28 (m, 2H), 7.49 (d, 1H), 7.76 (d, 1H), 7.84 (dd, 1H), 8.87 (s, 1H). LCMS (method 1): Rₜ = 1.18 min, MS (ESIpos) m/z = 474 (M + H)⁺. |
| 258 | 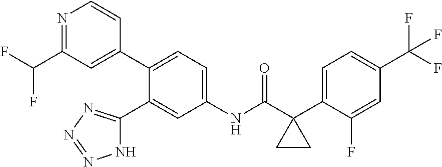 [SMILES: O=C(Nc1ccc(-c2ccnc(C(F)F)c2)c(-c2nnn[nH]2)c1)C1(c2ccc(C(F)(F)F)cc2F)CC1] | N-{4-[2- (difluoromethyl) pyridin-4-yl]-3- (1H-tetrazol-5- yl)phenyl}-1-[2- fluoro-4- (trifluoromethyl) phenyl]cyclopro- panecarboxamide | ¹H NMR (500 MHz, DMSO-d6) δ [ppm] 9.32 (s, 1H), 8.57 (d, J = 5.1 Hz, 1H), 7.98 (d, J = 2.1 Hz, 1H), 7.91 (dd, J = 8.6, 2.2 Hz, 1H), 7.75-7.65 (m, 2H), 7.63- 7.58 (m, 1H), 7.56 (d, J = 8.5 Hz, 1H), 7.34 (s, 1H), 7.26-7.18 (m, 1H), 6.91 (t, J = 54.9 Hz, 1H), 1.70-1.61 (m, 2H), 1.33-1.21 (m, 2H). LCMS (Analytical Method F): Rt = |
| | | | 3.55 mins; MS (ESIpos) m/z = 519 |
| | | | (M + H)⁺. |
| 259 | 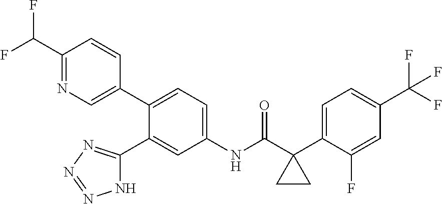 [SMILES: O=C(Nc1ccc(-c2ccc(C(F)F)nc2)c(-c2nnn[nH]2)c1)C1(c2ccc(C(F)(F)F)cc2F)CC1] | N-{4-[6- (difluoromethyl) pyridin-3-yl]-3- (1H-tetrazol-5- yl)phenyl}-1-[2- fluoro-4- (trifluoromethyl) phenyl]cyclopro- panecarboxamide | ¹H NMR (500 MHz, DMSO-d6) δ [ppm] 9.33 (s, 1H), 8.41 (d, J = 1.5 Hz, 1H), 8.01 (s, 1H), 7.92 (dd, J = 8.5, 2.2 Hz, 1H), 7.75- 7.59 (m, 5H), 7.57 (d, J = 8.5 Hz, 1H), 6.96 (t, J = 55.0 Hz, 1H), 1.74-1.59 (m, 2H), 1.34-1.19 (m, 2H). LCMS (Analytical Method F): Rt = 3.59 mins; MS (ESIpos) m/z = 519.1 (M + H)⁺. |
| 260 | 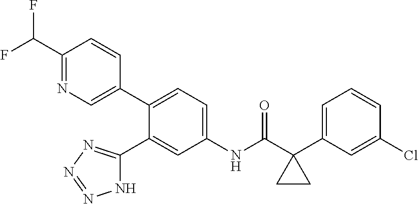 [SMILES: O=C(Nc1ccc(-c2ccc(C(F)F)nc2)c(-c2nnn[nH]2)c1)C1(c2cccc(Cl)c2)CC1] | 1-(3- chlorophenyl)- N-{4-[6- (difluoromethyl) pyridin-3-yl]-3- (1H-tetrazol-5- yl)phenyl}cyclopro- panecarboxa- mide | ¹H NMR (500 MHz, DMSO-d6) δ [ppm] 9.60 (s, 1H), 8.41 (d, J = 1.4 Hz, 1H), 8.07 (s, 1H), 7.94 (dd, J = 8.5, 2.2 Hz, 1H), 7.66 (dd, J = 8.1, 2.0 Hz, 1H), 7.63 (d, J = 8.0 Hz, 1H), 7.57 (d, J = 8.5 Hz, 1H), 7.47-7.43 (m, 1H), 7.42-7.35 (m, 3H), 6.96 (t, J = 55.0 Hz, 1H), 1.57-1.45 (m, 2H), 1.27-1.15 (m, 2H). LCMS (Analytical Method F): Rt = 3.45 mins; MS (ESIpos) m/z = 467.1 (M + H)⁺. |
| 261 | 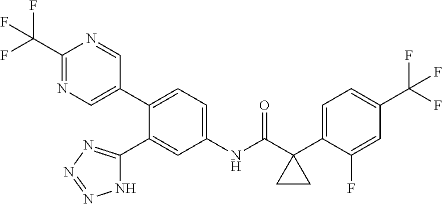 [SMILES: O=C(Nc1ccc(-c2cnc(C(F)(F)F)nc2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[2- (trifluoromethyl) pyrimidin-5- yl]phenyl}cyclopro- panecarboxa- mide | ¹H NMR (500 MHz, DMSO-d6) δ [ppm] 9.34 (s, 1H), 8.83 (s, 2H), 8.14 (d, J = 1.8 Hz, 1H), 7.88 (dd, J = 8.5, 2.1 Hz, 1H), 7.76- 7.66 (m, 2H), 7.62 (d, J = 8.1 Hz, 1H), 7.54 (d, J = 8.5 Hz, 1H), 1.74-1.58 (m, 2H), 1.33-1.23 (m, 2H). LCMS (Analytical Method F): Rt = 3.78 mins; MS (ESIpos) m/z = 538 (M + H)⁺. |
| 262 | 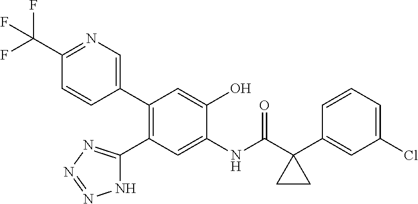 [SMILES: O=C(Nc1cc(-c2nnn[nH]2)c(-c2ccc(C(F)(F)F)nc2)cc1O)C1(c2cccc(Cl)c2)CC1] | 1-(3- chlorophenyl)- N-{2-hydroxy-5- (1H-tetrazol-5- yl)-4-[6- (trifluoromethyl) pyridin-3- yl]phenyl}cyclopro- panecarboxa- mide | ¹H NMR (400 MHz, DMSO-d6) δ [ppm] 1.24-1.27 (m, 2H), 1.54- 1.57 (m, 2H), 6.91 (s, 1H), 7.49- 7.57 (m, 3H), 7.66-7.67 (m, 1H), 7.74 (dd, 1H), 7.84 (d, 1H), 8.07 (s, 1H), 8.47 (d, 1H), 8.55 (s, br, 1H), 11.04 (s, 1H). LCMS (method 1): Rₜ = 1.16 min; MS (ESIpos) m/z = 501 (M + H)⁺ |
| 263 | 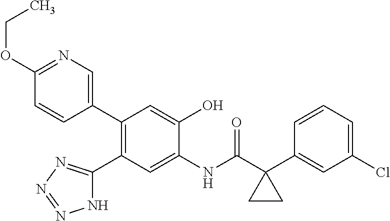 [SMILES: CCOc1ccc(-c2cc(O)c(NC(=O)C3(c4cccc(Cl)c4)CC3)cc2-c2nnn[nH]2)cn1] | 1-(3- chlorophenyl)- N-[4-(6- ethoxypyridin-3- yl)-2-hydroxy-5- (1H-tetrazol-5- yl)phenyl]cyclopro- panecarboxa- mide | ¹H NMR (400 MHz, DMSO-d6) δ [ppm] 1.22-1.25 (m, 2H), 1.30 (t, 3H), 1.52-1.55 (m, 2H), 4.27 (q, 2H), 6.69 (d, 1H), 6.83 (s, 1H), 6.27 (dd, 1H), 7.49-7.56 (m, 3H), 7.65-7.66 (m, 1H), 7.83 (d, 1H), 8.01 (s, 1H), 8.40 (s, 1H), 10.83 (s, 1H). LCMS (method 1): Rₜ = 1.12 min; MS (ESIpos) m/z = 477 (M + H)⁺ |
| 264 | 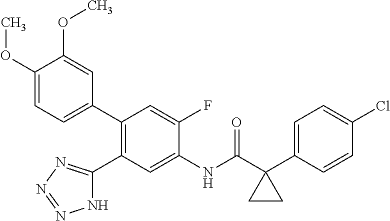 [SMILES: COc1ccc(-c2cc(F)c(NC(=O)C3(c4ccc(Cl)cc4)CC3)cc2-c2nnn[nH]2)cc1OC] | 1-(4- Chlorophenyl)- N-[5-fluoro- 3′,4′-dimethoxy- 2-(1H-tetrazol- 5-yl)biphenyl-4- yl]cyclopropane carboxamide | ¹H NMR (400 MHz, DMSO-d6) δ [ppm] 1.15-1.18 (m, 2H), 1.49- 1.52 (m, 2H), 3.59 (s, 3H), 3.73 (s, 3H), 6.58 (dd, 1H), 6.63 (d, 1H), 6.88 (d, 1H), 7.45-7.51 (m, 5H), 7.77 (d, 1H), 8.79 (s, 1H). LCMS (method 1): Rt = 1.26 min; MS (ESIpos) m/z = 494 (M + H)⁺. |
| 265 | 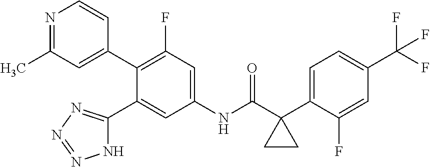 [SMILES: Cc1cc(-c2c(F)cc(NC(=O)C3(c4ccc(C(F)(F)F)cc4F)CC3)cc2-c2nnn[nH]2)ccn1] | N-[3-fluoro-4- (2- methylpyridin- 4-yl)-5-(1H- tetrazol-5- yl)phenyl]-1-[2- fluoro-4- (trifluoromethyl) phenyl]cyclopro- panecarboxamide | ¹H NMR (500 MHz, DMSO-d6) δ [ppm] 9.42 (s, 1H), 8.35 (d, J = 5.1 Hz, 1H), 7.86 (dd, J = 12.4, 2.0 Hz, 1H), 7.82 (d, J = 1.7 Hz, 1H), 7.74-7.66 (m, 2H), 7.64- 7.59 (m, 1H), 7.03 (s, 1H), 6.86 (d, J = 5.1 Hz, 1H), 2.41 (s, 3H), 1.74-1.61 (m, 2H), 1.35-1.21 (m, 2H). LCMS (Analytical Method F): Rt = |
| | | | 2.71 mins; MS (ESIpos) m/z = |
| | | | 501.1 (M + H)⁺. |
| 266 | 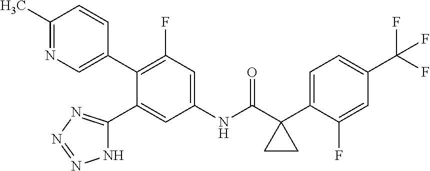 [SMILES: Cc1ccc(-c2c(F)cc(NC(=O)C3(c4ccc(C(F)(F)F)cc4F)CC3)cc2-c2nnn[nH]2)cn1] | N-[3-fluoro-4- (6- methylpyridin- 3-yl)-5-(1H- tetrazol-5- yl)phenyl]-1-[2- fluoro-4- (trifluoromethyl) phenyl]cyclopro- panecarboxamide | 1H NMR (500 MHz, DMSO-d6) δ [ppm] 9.42 (s, 1H), 8.15 (s, 1H), 7.87 (dd, J = 12.2, 1.8 Hz, 1H), 7.82 (s, 1H), 7.75-7.66 (m, 2H), 7.62 (d, J = 8.1 Hz, 1H), 7.41 (dd, J = 8.0, 1.7 Hz, 1H), 7.22 (d, J = 8.0 Hz, 1H), 2.46 (s, 3H), 1.71-1.64 (m, 2H), 1.32-1.25 (m, 2H). LCMS (Analytical Method F): Rt = 2.80 mins; MS (ESIpos) m/z = |
| | | | 501.1 (M + H)⁺. |
| 267 | 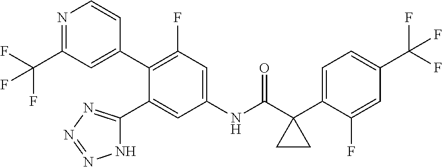 [SMILES: O=C(Nc1cc(F)c(-c2ccnc(C(F)(F)F)c2)c(-c2nnn[nH]2)c1)C1(c2ccc(C(F)(F)F)cc2F)CC1] | N-{3-fluoro-5- (1H-tetrazol-5- yl)-4-[2- (trifluoromethyl) pyridin-4- yl]phenyl}-1-[2- fluoro-4- (trifluoromethyl) phenyl]cyclopro- panecarboxamide | ¹H NMR (500 MHz, DMSO-d6) δ [ppm] 9.51 (s, 1H), 8.73 (d, J = 5.0 Hz, 1H), 7.97 (s, 1H), 7.93 (dd, J = 12.4, 2.0 Hz, 1H), 7.75- 7.67 (m, 3H), 7.65-7.60 (m, 1H), 7.48 (d, J = 4.9 Hz, 1H), 1.75-1.63 (m, 2H), 1.36-1.25 (m, 2H). LCMS (Analytical Method F): Rt 3.96 mins; MS (ESIpos) m/z = 555.1 (M + H)⁺. |
| 268 | 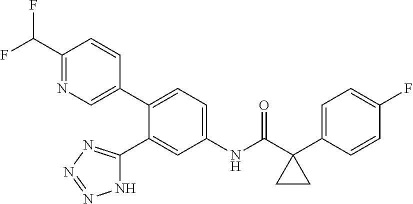 [SMILES: O=C(Nc1ccc(-c2ccc(C(F)F)nc2)c(-c2nnn[nH]2)c1)C1(c2ccc(F)cc2)CC1] | N-{4-[6- (difluoromethyl) pyridin-3-yl]-3- (1H-tetrazol-5- yl)phenyl}-1-(4- fluorophenyl)cy- cloproapnecarbox- amide | ¹H NMR (500 MHz, DMSO-d6) δ 9.39 (s, 1H), 8.44-8.33 (m, 1H), 8.09-7.99 (m, 1H), 7.99-7.91 (m, 1H), 7.70-7.51 (m, 3H), 7.51-7.39 (m, 2H), 7.26-7.13 (m, 2H), 6.96 (t, J = 55.0 Hz, 1H), 1.54-1.40 (m, 2H), 1.19- 1.08 (m, 2H). LCMS (Analytical Method F): Rt = 3.25 mins, MS (ESIpos): m/z = 451 (M + H)⁺. |
| 269 | 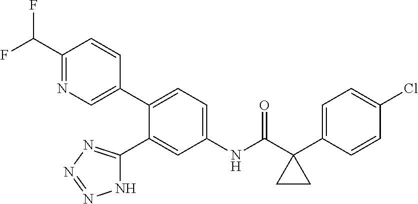 [SMILES: O=C(Nc1ccc(-c2ccc(C(F)F)nc2)c(-c2nnn[nH]2)c1)C1(c2ccc(Cl)cc2)CC1] | 1-(4- chlorophenyl)- N-{4-[6- (difluoromethyl) pyridin-3-yl]-3- (1H-tetrazol-5- yl)phenyl}cyclopro- panecarboxa- mide | ¹H NMR (500 MHz, DMSO-d6) δ 9.49 (s, 1H), 8.44-8.36 (m, 1H), 8.09-8.01 (m, 1H), 7.94 (dd, J = 8.5, 2.2 Hz, 1H), 7.68-7.60 (m, 2H), 7.60-7.50 (m, 1H), 7.43 (s, 4H), 6.96 (t, J = 55.0 Hz, 1H), 1.56-1.44 (m, 2H), 1.21-1.11 (m, 2H). LCMS (Analytical Method F): Rt = 3.49 mins, MS (ESIpos): m/z = 467 (M + H)⁺. |
| 270 | 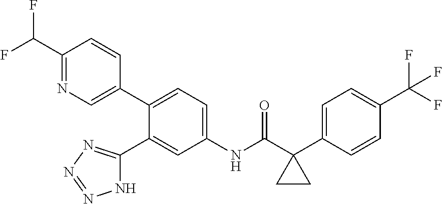 [SMILES: O=C(Nc1ccc(-c2ccc(C(F)F)nc2)c(-c2nnn[nH]2)c1)C1(c2ccc(C(F)(F)F)cc2)CC1] | N-{4-[6- (difluoromethyl) pyridin-3-yl]-3- (1H-tetrazol-5- yl)phenyl}-1-[4- (trifluoromethyl) phenyl]cyclopro- panecarboxamide | ¹H NMR (500 MHz, DMSO-d6) δ 9.57 (s, 1H), 8.39-8.30 (m, 1H), 7.96 (s, 1H), 7.82 (d, J = 8.4 Hz, 1H), 7.72 (d, J = 8.3 Hz, 2H), 7.69-7.64 (m, 1H), 7.61 (d, J = 8.2 Hz, 2H), 7.58-7.52 (m, 1H), 7.41-7.32 (m, 1H), 6.93 (t, J = 55.1 Hz, 1H), 1.58-1.54 (m, 2H), 1.25-1.20 (m, 2H). LCMS (Analytical Method F): Rt = 3.57 mins, MS (ESIpos): m/z = 501 (M + H)⁺. |
| 271 | 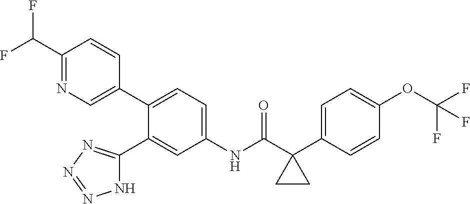 [SMILES: O=C(Nc1ccc(-c2ccc(C(F)F)nc2)c(-c2nnn[nH]2)c1)C1(c2ccc(OC(F)(F)F)cc2)CC1] | N-{4-[6- (difluoromethyl) pyridin-3-yl]-3- (1H-tetrazol-5- yl)phenyl}-1-[4- (trifluoromethoxy) phenyl]cyclo- propanecarboxa- mide | ¹H NMR (500 MHz, DMSO-d6) δ 9.60 (s, 1H), 8.44-8.37 (m, 1H), 8.10-8.03 (m, 1H), 7.98-7.90 (m, 1H), 7.68-7.59 (m, 2H), 7.59-7.49 (m, 3H), 7.40-7.31 (m, 2H), 6.96 (t, J = 55.0 Hz, 1H), 1.59-1.45 (m, 2H), 1.27- 1.14 (m, 2H). LCMS (Analytical Method F): Rt = 3.65 mins, MS (ESIpos): m/z = 517 (M + H)⁺. |
| 272 | 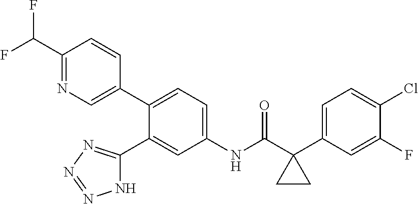 [SMILES: O=C(Nc1ccc(-c2ccc(C(F)F)nc2)c(-c2nnn[nH]2)c1)C1(c2ccc(Cl)c(F)c2)CC1] | 1-(4-chloro-3- fluorophenyl)- N-{4-[6- (difluoromethyl) pyridin-3-yl]-3- (1H-tetrazol-5- yl)phenyl}cyclopro- panecarboxa- mide | ¹H NMR (500 MHz, DMSO-d6) δ 9.47 (s, 1H), 8.41 (s, 1H), 8.09- 8.00 (m, 1H), 8.00-7.87 (m, 1H), 7.68, 7.60 (m, 2H), 7.60-7.52 (m, 2H), 7.50-7.43 (m, 1H), 7.32-7.25 (m, 1H), 6.96 (t, J = 55.0 Hz, 1H), 1.59-1.43 (m, 2H), 1.27-1.15 (m, 2H). LCMS (Analytical Method F): Rt = 3.52 mins, MS (ESIpos): m/z = 485 (M + H)⁺. |
| 273 | 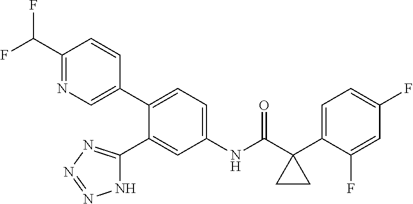 [SMILES: O=C(Nc1ccc(-c2ccc(C(F)F)nc2)c(-c2nnn[nH]2)c1)C1(c2ccc(F)cc2F)CC1] | N-{4-[6- (difluoromethyl) pyridin-3-yl]-3- (1H-tetrazol-5- yl)phenyl}-1- (2,4- difluorophenyl)cy- clopropanecar- boxamide | ¹H NMR (500 MHz, DMSO-d6) δ 9.19 (s, 1H), 8.44-8.34 (m, 1H), 8.04-7.97 (m, 1H), 7.96-7.89 (m, 1H), 7.70-7.59 (m, 2H), 7.60-7.48 (m, 2H), 7.30-7.20 (m, 1H), 7.15-7.08 (m, 1H), 6.96 (t, J = 55.0 Hz, 1H), 1.65- 1.56 (m, 2H), 1.22-1.12 (m, 2H). LCMS (Analytical Method F): Rt = 3.24 mins, MS (ESIpos): m/z = 469 (M + H)⁺. |
| 274 | 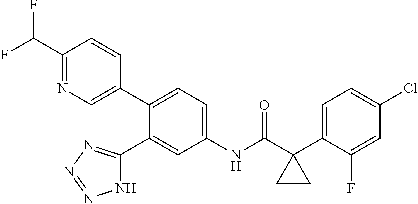 [SMILES: O=C(Nc1ccc(-c2ccc(C(F)F)nc2)c(-c2nnn[nH]2)c1)C1(c2ccc(Cl)cc2F)CC1] | 1-(4-chloro-2- fluorophenyl)- N-{4-[6- (difluoromethyl) pyridin-3-yl]-3- (1H-tetrazol-5- yl)phenyl}cyclopro- panecarboxa- mide | ¹H NMR (500 MHz, DMSO-d6) δ 9.24 (s, 1H), 8.43-8.37 (m, 1H), 8.06-7.97 (m, 1H), 7.97-7.88 (m, 1H), 7.70-7.59 (m, 2H), 7.59-7.54 (m, 1H), 7.54-7.47 (m, 1H), 7.47-7.41 (m, 1H), 7.37-7.28 (m, 1H), 6.96 (t, J = 55.0 Hz, 1H), 1.68-1.55 (m, 2H), 1.26-1.13 (m, 2H). LCMS (Analytical Method F): Rt = 3.47 mins, MS (ESIpos): m/z = 485 (M + H)⁺. |
| 275 | 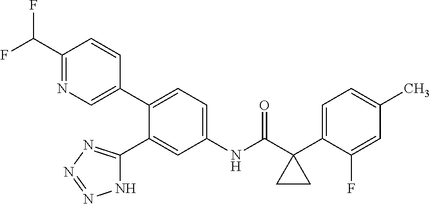 [SMILES: Cc1ccc(C2(C(=O)Nc3ccc(-c4ccc(C(F)F)nc4)c(-c4nnn[nH]4)c3)CC2)c(F)c1] | N-{4-[6- (difluoromethyl) pyridin-3-yl]-3- (1H-tetrazol-5- yl)phenyl}-1-(2- fluoro-4- methylphenyl)cy- clopropanecar- boxamide | ¹H NMR (500 MHz, DMSO-d6) δ 9.14 (s, 1H), 8.44-8.34 (m, 1H), 8.05-7.97 (m, 1H), 7.97-7.88 (m, 1H), 7.70-7.59 (m, 2H), 7.59-7.51 (m, 1H), 7.40-7.28 (m, 1H), 7.14-6.80 (m, 3H), 2.34 (s, 3H), 1.61-1.53 (m, 2H), 1.17-1.09 (m, 2H). LCMS (Analytical Method F): Rt = 3.41 mins, MS (ESIpos): m/z = 465 (M + H)⁺. |
| 276 | 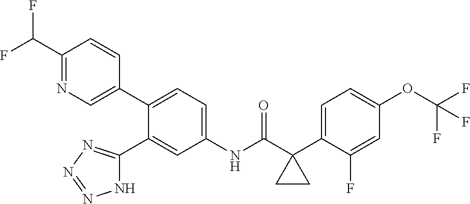 [SMILES: O=C(Nc1ccc(-c2ccc(C(F)F)nc2)c(-c2nnn[nH]2)c1)C1(c2ccc(OC(F)(F)F)cc2F)CC1] | N-{4-[6- (difluoromethyl) pyridin-3-yl]-3- (1H-tetrazol-5- yl)phenyl}-1-[2- fluoro-4- (trifluoromethoxy) phenyl]cyclo- propanecarboxa- mide | ¹H NMR (500 MHz, DMSO-d6) δ 9.29 (s, 1H), 8.43-8.32 (m, 1H), 8.03-7.97 (m, 1H), 7.95-7.86 (m, 1H), 7.70-7.58 (m, 3H), 7.55 (d, J = 8.5 Hz, 1H), 7.44- 7.35 (m, 1H), 7.29-7.21 (m, 1H), 6.96 (t, J = 55.0 Hz, 1H), 1.67-1.57 (m, 2H), 1.26-1.19 (m, 2H). LCMS (Analytical Method F): Rt = 3.67 mins, MS (ESIpos): m/z = 534 (M + H)⁺. |
| 277 | 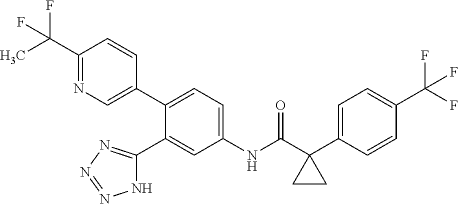 [SMILES: CC(F)(F)c1ccc(-c2ccc(NC(=O)C3(c4ccc(C(F)(F)F)cc4)CC3)cc2-c2nnn[nH]2)cn1] | N-{4-[6-(1,1- difluoroethyl)py- ridin-3-yl]-3- (1H-tetrazol-5- yl)phenyl}-1-[4- (trifluoromethyl) phenyl]cyclopro- panecarboxamide | ¹H NMR (250 MHz, DMSO-d6) δ 1.20-1.31 (m, 2H), 1.52-1.62 (m, 2H), 2.00 (t, J = 19.1 Hz, 3H), 7.52-7.66 (m, 5H), 7.73 (m, 2H), 7.94 (m, 1H), 8.06 (m, 1H), 8.40 (s, 1H), 9.69 (s, 1H). LCMS (Analytical Method B): Rt = 1.12 mins; m/z (ESI) = 514.9 (M + H)⁺. |
| 278 | 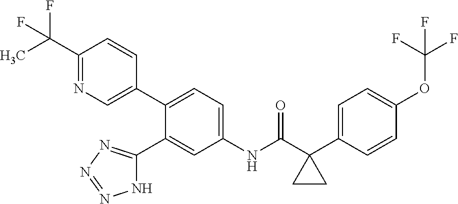 [SMILES: CC(F)(F)c1ccc(-c2ccc(NC(=O)C3(c4ccc(OC(F)(F)F)cc4)CC3)cc2-c2nnn[nH]2)cn1] | N-{4-[6-(1,1- difluroethyl)py- ridin-3-yl]-3- (1H-tetrazol-5- yl)phenyl}-1-[4- (trifluoromethoxy) phenyl]cyclo- propanecarboxa- mide | ¹H NMR (250 MHz, DMSO-d6) δ 1.16-1.24 (m, 2H), 1.48-1.60 (m, 2H), 2.00 (t, J = 19.1 Hz, 3H), 7.22-7.46 (m, 2H), 7.48-7.59 (m, 3H), 7.63 (m, 2H), 7.93 (m, 1H), 8.05 (m, 1H), 8.40 (s, 1H), 9.59 (s, 1H). LCMS (Analytical Method B): Rt = 1.13 mins; m/z (ESI) = 530.1 (M + H)⁺. |
| 279 | 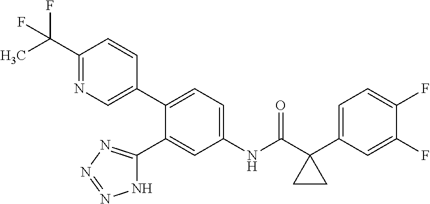 [SMILES: CC(F)(F)c1ccc(-c2ccc(NC(=O)C3(c4ccc(F)c(F)c4)CC3)cc2-c2nnn[nH]2)cn1] | N-{4-[6-(1,1- difluoroethyl)py- ridin-3-yl]-3- (1H-tetrazol-5- yl)phenyl}-1- (3,4- difluorophenyl)cy- clopropanecar- boxamide | ¹H NMR (250 MHz, DMSO-d6) δ 3.50-3.64 (m, 2H), 3.80-3.97 (m, 2H), 4.37 (t, J = 19.1 Hz, 3H), 9.58-10.05 (m, 6H), 10.31 (dd, J = 8.5, 2.2 Hz, 1H), 10.40 (d, J = 2.0 Hz, 1H), 10.77 (s, 1H), 11.74 (s, 1H). LCMS (Analytical Method D): Rt = 4.18 mins; m/z (ESI) = 483.15 (M + H)⁺. |
| 280 | 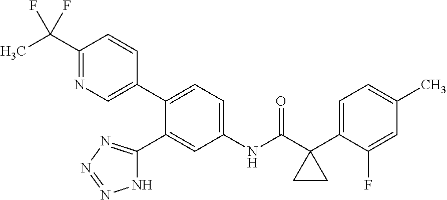 [SMILES: Cc1ccc(C2(C(=O)Nc3ccc(-c4ccc(C(C)(F)F)nc4)c(-c4nnn[nH]4)c3)CC2)c(F)c1] | N-{4-[6-(1,1- difluoroethyl)py- ridin-3-yl]-3- (1H-tetrazol-5- yl)phenyl}-1-(2- fluoro-4- methylphenyl)cy- clopropanecar- boxamide | ¹H NMR (500 MHz, DMSO-d6) δ 9.15 (s, 1H), 8.40 (s, 1H), 8.01 (s, 1H), 7.98-7.91 (m, 1H), 7.67- 7.58 (m, 2H), 7.59-7.51 (m, 1H), 7.40-7.31 (m, 1H), 7.08-7.01 (m, 2H), 2.34 (s, 3H), 2.00 (t, J = 19.1 Hz, 3H), 1.62-1.56 (m, 2H), 1.17-1.11 (m, 2H). LCMS (Analytical Method F): Rt = 3.59 mins, MS (ESIpos): m/z = 479 (M + H)⁺. |
| 281 | 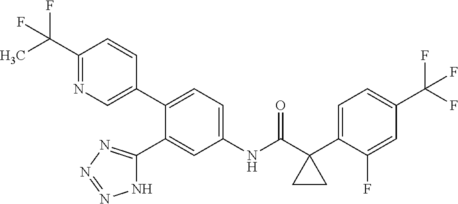 [SMILES: CC(F)(F)c1ccc(-c2ccc(NC(=O)C3(c4ccc(C(F)(F)F)cc4F)CC3)cc2-c2nnn[nH]2)cn1] | N-{4-[6-(1,1- difluoroethyl)py- ridin-3-yl]-3- (1H-tetrazol-5- yl)phenyl}-1-[2- fluoro-4- (trifluoromethyl) phenyl]cyclopro- panecarboxamide | ¹H NMR (250 MHz, DMSO-d6) δ 1.18-1.33 (m, 2H), 1.60-1.72 (m, 2H), 2.00 (t, J = 19.1 Hz, 3H), 7.43-7.77 (m, 6H), 7.88 (dd, J = 8.5, 2.2 Hz, 1H), 7.97 (d, J = 2.1 Hz, 1H), 8.39 (s, 1H), 9.29 (s, 1H). LCMS (Analytical Method F): Rt = 3.73 mins; m/z (ESI) = 533.1 (M + H)⁺. |
| 282 | 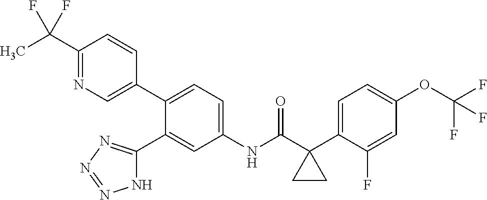 [SMILES: CC(F)(F)c1ccc(-c2ccc(NC(=O)C3(c4ccc(OC(F)(F)F)cc4F)CC3)cc2-c2nnn[nH]2)cn1] | N-{4-[6-(1,1- difluoroethyl)py- ridin-3-yl]-3- (1H-tetrazol-5- yl)phenyl}-1-[2- fluoro-4- (trifluoromethoxy) phenyl]cyclo- propanecarboxa- mide | ¹H NMR (500 MHz, DMSO-d6) δ 9.28 (s, 1H), 8.42-8.38 (m, 1H), 8.03-7.96 (m, 1H), 7.96-7.88 (m, 1H), 7.66-7.53 (m, 4H), 7.43- 7.35 (m, 1H), 7.30-7.22 (m, 1H), 2.00 (t, J = 19.1 Hz, 3H), 1.65-1.59 (m, 2H), 1.25-1.19 (m, 2H). LCMS (Analytical Method F): Rt = 3.83 mins, MS (ESIpos): m/z = 549 (M + H)⁺. |
| 283 | 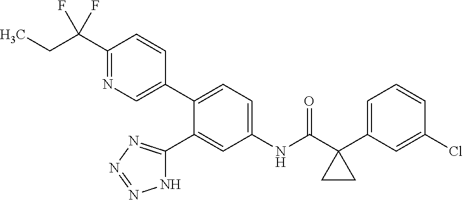 [SMILES: CCC(F)(F)c1ccc(-c2ccc(NC(=O)C3(c4cccc(Cl)c4)CC3)cc2-c2nnn[nH]2)cn1] | 1-(3- chlorophenyl)- N-{4-[6-(1,1- difluoropropyl)py- ridin-3-yl]-3- (1H-tetrazol-5- yl)phenyl}cyclopro- panecarboxa- mide | ¹H NMR (500 MHz, DMSO-d6) δ 9.60 (s, 1H), 8.41 (s, 1H), 8.06 (s, 1H), 8.01-7.89 (m, 1H), 7.68- 7.54 (m, 3H), 7.51-7.30 (m, 4H), 2.36-2.24 (m, 2H), 1.56-1.47 (m, 2H), 1.27-1.18 (m, 2H), 0.93 (t, J = 7.5 Hz, 3H). LCMS (Analytical Method F): Rt = 3.80 mins, MS (ESIpos): m/z = 495 (M + H)⁺. |
| 284 | 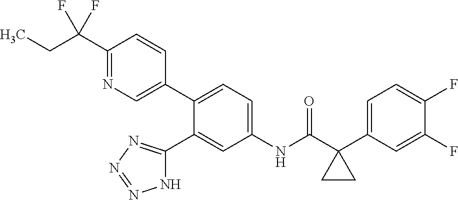 [SMILES: CCC(F)(F)c1ccc(-c2ccc(NC(=O)C3(c4ccc(F)c(F)c4)CC3)cc2-c2nnn[nH]2)cn1] | 1-(3,4- difluorophenyl)- N-{4-[6-(1,1- difluoropropyl)py- ridin-3-yl]-3- (1H-tetrazol-5- yl)phenyl}cyclopro- panecarboxa- mide | ¹H NMR (500 MHz, DMSO-d6) δ 9.37 (s, 1H), 8.40 (s, 1H), 8.06- 7.99 (m, 1H), 7.98-7.90 (m, 1H), 7.64-7.54 (m, 3H), 7.54-7.46 (m, 1H), 7.46-7.38 (m, 1H), 7.32- 7.25 (m, 1H), 2.36-2.24 (m, 2H), 1.51 (m, 2H), 1.23-1.15 (m, 2H), 0.91 (t, J = 7.5 Hz, 3H). LCMS (Analytical Method F): Rt = 3.67 mins, MS (ESIpos): m/z = 497 (M + H)⁺. |
| 285 | 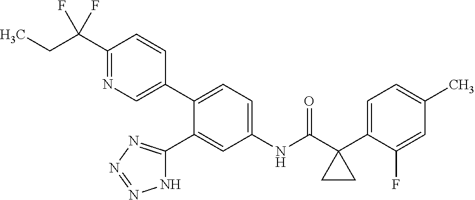 [SMILES: CCC(F)(F)c1ccc(-c2ccc(NC(=O)C3(c4ccc(C)cc4F)CC3)cc2-c2nnn[nH]2)cn1] | N-{4-[6-(1,1- difluoropropyl)py- ridin-3-yl]-3- (1H-tetrazol-5- yl)phenyl}-1-(2- fluoro-4- methylphenyl)cy- clopropanecar- boxamide | ¹H NMR (500 MHz, DMSO-d6) δ 9.14 (s, 1H), 8.45-8.36 (m, 1H), 8.03-7.98 (m, 1H), 7.97-7.90 (m, 1H), 7.64-7.58 (m, 2H), 7.58- 7.50 (m, 1H), 7.38-7.31 (m, 1H), 7.08-7.01 (m, 2H), 2.42- 2.24 (m, 5H), 1.63-1.52 (m, 2H), 1.19-1.09 (m, 2H), 0.91 (t, J = 7.5 Hz, 3H). LCMS (Analytical Method F): Rt = 3.75 mins, MS (ESIpos): m/z = 493 (M + H)⁺. |
| 286 | 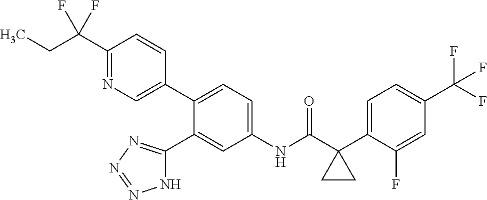 [SMILES: CCC(F)(F)c1ccc(-c2ccc(NC(=O)C3(c4ccc(C(F)(F)F)cc4F)CC3)cc2-c2nnn[nH]2)cn1] | N-{4-[6-(1,1- difluoropropyl)py- ridin-3-yl]-3- (1H-tetrazol-5- yl)phenyl}-1-[2- fluoro-4- (trifluoromethyl) phenyl]cyclopro- panecarboxamide | ¹H NMR (500 MHz, DMSO-d6) δ 9.32 (s, 1H), 8.44-8.35 (m, 1H), 8.03-7.96 (m, 1H), 7.96-7.87 (m, 1H), 7.76-7.66 (m, 2H), 7.65- 7.52 (m, 4H), 2.39-2.23 (m, 2H), 1.74-1.60 (m, 2H), 1.34- 1.18 (m, 2H), 0.91 (t, J = 7.4 Hz, 3H). LCMS (Analytical Method F): Rt = 3.89 mins, MS (ESIpos): m/z = 547 (M + H)⁺. |
| 287 | 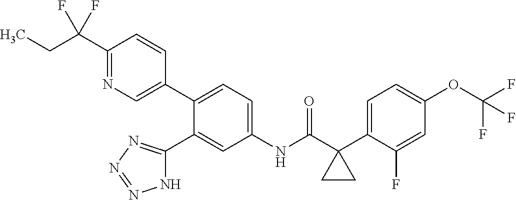 [SMILES: CCC(F)(F)c1ccc(-c2ccc(NC(=O)C3(c4ccc(OC(F)(F)F)cc4F)CC3)cc2-c2nnn[nH]2)cn1] | N-{4-[6-(1,1- difluoropropyl)py- ridin-3-yl]-3- (1H-tetrazol-5- yl)phenyl}-1-[2- fluoro-4- (trifluoromethoxy) phenyl]cyclo- propanecarboxa- mide | ¹H NMR (500 MHz, DMSO-d6) δ 9.28 (s, 1H), 8.45-8.35 (m, 1H), 8.04-7.96 (m, 1H), 7.95-7.88 (m, 1H), 7.66-7.58 (m, 3H), 7.58- 7.53 (m, 1H), 7.43-7.35 (m, 1H), 7.29-7.21 (m, 1H), 2.36- 2.24 (m, 2H), 1.67-1.60 (m, 2H), 1.27-1.19 (m, 2H), 0.92 (t, J = 7.5 Hz, 3H). LCMS (Analytical Method F): Rt = 3.96 mins, MS (ESIpos): m/z = 563 (M + H)⁺. |
| 288 | 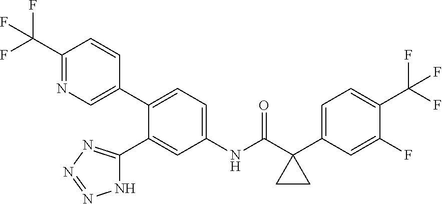 [SMILES: O=C(Nc1ccc(-c2ccc(C(F)(F)F)nc2)c(-c2nnn[nH]2)c1)C1(c2ccc(C(F)(F)F)c(F)c2)CC1] | 1-[3-fluoro-4- (trifluoromethyl) phenyl]-N-{3- (1H-tetrazol-5- yl)-4-[6- (trifluoromethyl) pyridin-3- yl]phenyl}cyclopro- panecarboxa- mide | ¹H NMR (250 MHz, DMSO-d6) δ 1.27-1.35 (m, 2H), 1.53-1.65 (m, 2H), 7.44 (m, 1H), 7.53 (m, 1H), 7.60 (m, 1H), 7.76 (m, 2H), 7.85 (m, 1H), 7.95 (m, 1H), 8.10 (m, 1H), 8.52 (m, 1H), 9.68 (s, 1H). LCMS (Analytical Method F): Rt = 3.84 mins; m/z (ESI) = 537.1 (M + H)⁺. |
| 289 | 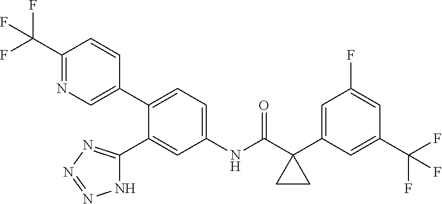 [SMILES: O=C(Nc1ccc(-c2ccc(C(F)(F)F)nc2)c(-c2nnn[nH]2)c1)C1(c2cc(F)cc(C(F)(F)F)c2)CC1] | 1-[3-fluoro-5- (trifluoromethyl) phenyl]-N-{3- (1H-tetrazol-5- yl)-4-[6- (trifluoromethyl) pyridin-3- yl]phenyl}cyclopro- panecarboxa- mide | ¹H NMR (250 MHz, DMSO-d6) δ 1.27-1.37 (m, 2H), 1.54-1.63 (m, 2H), 7.52-7.68 (m, 4H), 7.70- 7.79 (m, 1H), 7.85 (d, J = 8.2 Hz, 1H), 7.93 (dd, J = 8.5, 2.2 Hz, 1H), 8.09 (d, J = 2.0 Hz, 1H), 8.52 (d, J = 1.9 Hz, 1H), 9.62 (s, 1H). LCMS (Analytical Method F): Rt = 3.83 mins; m/z (ESI) = 537.1 (M + H)⁺. |
| 290 | 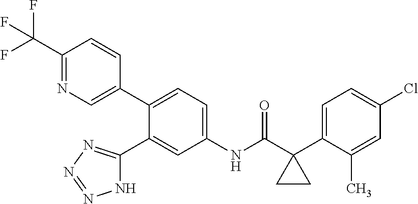 [SMILES: Cc1cc(Cl)ccc1C1(C(=O)Nc2ccc(-c3ccc(C(F)(F)F)nc3)c(-c3nnn[nH]3)c2)CC1] | 1-(4-chloro-2- methylphenyl)- N-{3-(1H- tetrazol-5-yl)-4- [6- (trifluoromethyl) pyridin-3- yl]phenyl}cyclopro- panecarboxa- mide | ¹H NMR (250 MHz, DMSO-d6) δ [ppm] 1.00-1.17 (m, 2H), 1.52- 1.70 (m, 2H), 2.33 (s, 3H), 7.23- 7.35 (m, 2H), 7.42 (d, J = 8.2 Hz, 1H), 7.57 (d, J = 8.5 Hz, 1H), 7.71- 7.79 (m, 1H), 7.84 (d, J = 8.2 Hz, 1H), 7.92 (dd, J = 8.5, 2.2 Hz, 1H), 8.02 (d, J = 2.1 Hz, 1H), 8.51 (d, J = 1.9 Hz, 1H), 8.99 (s, 1H). LCMS (Analytical Method F): Rt = 3.88 mins; m/z (ESI) = 499.1 (M + H)⁺. |
| 291 | 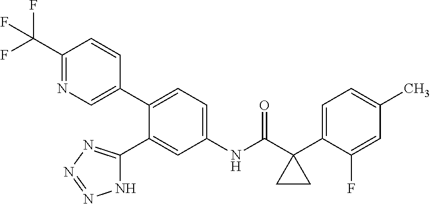 [SMILES: Cc1ccc(C2(C(=O)Nc3ccc(-c4ccc(C(F)(F)F)nc4)c(-c4nnn[nH]4)c3)CC2)c(F)c1] | 1-(2-fluoro-4- methylphenyl)- N-{3-(1H- tetrazol-5-yl)-4- [6- (trifluoromethyl) pyridin-3- yl]phenyl}cyclopro- panecarboxa- mide | ¹H NMR (250 MHz, DMSO-d6) δ [ppm] 1.15 (m, 2H), 1.58 (m, 2H), 2.34 (s, 3H), 6.97-7.11 (m, 2H), 7.27-7.42 (m, 1H), 7.56 (d, J = 8.5 Hz, 1H), 7.75 (dd, J = 8.0, 1.9 Hz, 1H), 7.84 (d, J = 7.8 Hz, 1H), 7.95 (dd, J = 8.5, 2.2 Hz, 1H), 8.05 (d, J = 2.1 Hz, 1H), 8.51 (d, J = 1.9 Hz, 1H), 9.17 (s, 1H). LCMS (Analytical Method F): Rt = 3.63 mins; m/z (ESI) = 483 (M + H)⁺. |
| 292 | 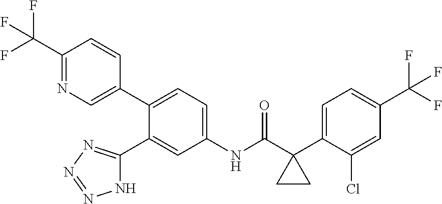 [SMILES: O=C(Nc1ccc(-c2ccc(C(F)(F)F)nc2)c(-c2nnn[nH]2)c1)C1(c2ccc(C(F)(F)F)cc2Cl)CC1] | 1-[2-chloro-4- (trifluoromethyl) phenyl]-N-{3- (1H-tetrazol-5- yl)-4-[6- (trifluoromethyl) pyridin-3- yl]phenyl}cyclopro- panecarboxa- mide | ¹H NMR (250 MHz, DMSO-d6) δ [ppm] 1.17-1.34 (m, 2H), 1.62- 1.80 (m, 2H), 7.58 (d, J = 8.5 Hz, 1H), 7.69-7.80 (m, 3H), 7.85 (d, J = 8.0 Hz, 1H), 7.88-7.96 (m, 2H), 8.03 (d, J = 1.9 Hz, 1H), 8.46- 8.57 (m, 1H), 9.17 (s, 1H). LCMS (Analytical Method F): Rt = 3.94 mins; m/z (ESI) = 553.2 (M + H)⁺. |
| 293 | 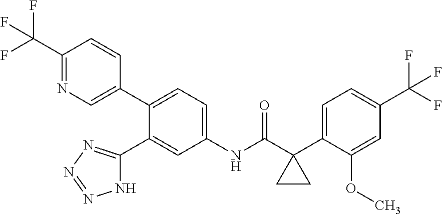 [SMILES: COc1cc(C(F)(F)F)ccc1C1(C(=O)Nc2ccc(-c3ccc(C(F)(F)F)nc3)c(-c3nnn[nH]3)c2)CC1] | 1-[2-methoxy-4- (trifluoromethyl) phenyl]-N-{3- (1H-tetrazol-5- yl)-4-[6- (trifluoromethyl) pyridin-3- yl]phenyl}cyclopro- panecarboxa- mide | ¹H NMR (250 MHz, DMSO-d6) δ [ppm] 1.05-1.17 (m, 2H), 1.51- 1.63 (m, 2H), 3.88 (s, 3H), 7.27- 7.37 (m, 2H), 7.49-7.60 (m, 2H), 7.75 (d, J = 8.1 Hz, 1H), 7.84 (d, J = 8.1 Hz, 1H), 7.92 (dd, J = 8.5, 2.2 Hz, 1H), 8.03 (d, J = 2.0 Hz, 1H), 8.51 (d, J = 1.9 Hz, 1H), 9.04 (s, 1H). LCMS (Analytical Method F): Rt = 3.82 mins; m/z (ESI) = 549.1 (M + H)⁺. |
| 294 | 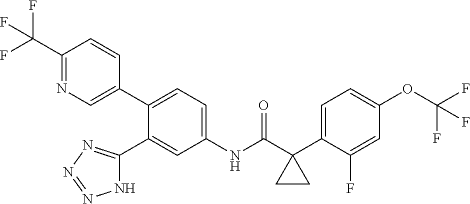 [SMILES: O=C(Nc1ccc(-c2ccc(C(F)(F)F)nc2)c(-c2nnn[nH]2)c1)C1(c2ccc(OC(F)(F)F)cc2F)CC1] | 1-[2-fluoro-4- (trifluoromethoxy) phenyl]-N-{3- (1H-tetrazol-5- yl)-4-[6- (trifluoromethyl) pyridin-3- yl]phenyl}cyclopro- panecarboxa- mide | ¹H NMR (500 MHz, DMSO-d6) δ [ppm] 9.31 (s, 1H), 8.52 (d, J = 1.8 Hz, 1H), 8.06 (s, 1H), 7.93 (dd, J = 8.5, 2.2 Hz, 1H), 7.85 (d, J = 8.2 Hz, 1H), 7.75 (dd, J = 8.1, 1.6 Hz, 1H), 7.66-7.55 (m, 2H), 7.39 (dd, J = 10.3, 1.8 Hz, 1H), 7.26 (d, J = 8.5 Hz, 1H), 1.70- 1.58 (m, 2H), 1.29-1.18 (m, 2H). LCMS (Analytical Method F): Rt = 3.87 mins; m/z (ESI) = 553.1 (M + H)⁺. |
| 295 | 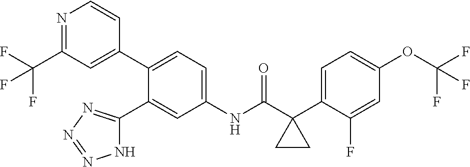 [SMILES: O=C(Nc1ccc(-c2ccnc(C(F)(F)F)c2)c(-c2nnn[nH]2)c1)C1(c2ccc(OC(F)(F)F)cc2F)CC1] | 1-[2-fluoro-4- (trifluoromethoxy) phenyl]-N-{3- (1H-tetrazol-5- yl)-4-[2- (trifluoromethyl) pyridin-4- yl]phenyl}cyclopro- panecarboxa- mide | ¹H NMR (500 MHz, DMSO-d6) δ [ppm] 9.29 (s, 1H), 8.66-8.60 (m, 1H), 8.02-7.98 (m, 1H), 7.93- 7.86 (m, 1H), 7.65-7.59 (m, 1H), 7.59-7.52 (m, 2H), 7.43-7.34 (m, 2H), 7.28-7.22 (m, 1H), 1.67- 1.58 (m, 2H), 1.25-1.20 (m, 2H). LCMS (Analytical Method F): Rt = 3.85 mins, MS (ESIpos): m/z = 553 (M + H)⁺. |
| 296 | 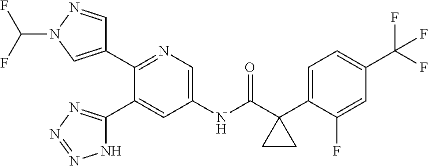 [SMILES: O=C(Nc1cnc(-c2cnn(C(F)F)c2)c(-c2nnn[nH]2)c1)C1(c2ccc(C(F)(F)F)cc2F)CC1] | N-{6-[1- (difluoromethyl)- 1H-pyrazol-4- yl]-5-(1H- tetrazol-5- yl)pyridin-3-yl}- 1-[2-fluoro-4- (trifluoromethyl) phenyl]cyclopro- panecarboxamide | 1H NMR (500 MHz, DMSO-d6) δ [ppm] 9.41 (s, 1H), 8.95 (d, J = 2.2 Hz, 1H), 8.20 (d, J = 2.1 Hz, 1H), 8.08 (s, 1H), 7.77 (t, J = 58.8 Hz, 1H), 7.75-7.68 (m, 2H), 7.64- 7.60 (m, 1H), 7.59 (s, 1H), 1.74- 1.62 (m, 2H), 1.35-1.22 (m, 2H). LCMS (Analytical Method F): Rt = 3.31 mins, m/z (ESIpos) = 509.1 (M + H)⁺. |
| 297 | 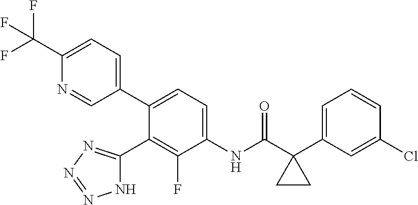 [SMILES: O=C(Nc1ccc(-c2ccc(C(F)(F)F)nc2)c(-c2nnn[nH]2)c1F)C1(c2cccc(Cl)c2)CC1] | 1-(3- chlorophenyl)- N-{2-fluoro-3- (1H-tetrazol-5- yl)-4-[6- (trifluoromethyl) pyridin-3- yl]phenyl}cyclopro- panecarboxa- mide | ¹H NMR (500 MHz, DMSO-d6) δ [ppm] 8.95 (s, 1H), 8.51 (s, 1H), 7.96 (m, 1H), 7.85 (d, J = 8.2 Hz, 1H), 7.76-7.70 (m, 1H), 7.52 (d, J = 6.1 Hz, 2H), 7.47-7.38 (m, 3H), 1.54 (m, 2H), 1.27-1.20 (m, 2H). LCMS (Analytical Method D) Rt = 4.53 min, MS (ESIpos): m/z = 503.0 (M + H)⁺. |
| 298 | 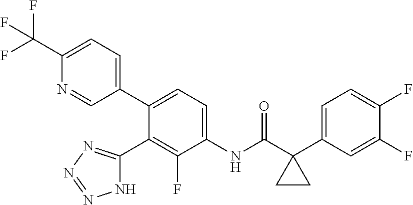 [SMILES: O=C(Nc1ccc(-c2ccc(C(F)(F)F)nc2)c(-c2nnn[nH]2)c1F)C1(c2ccc(F)c(F)c2)CC1] | 1-(3,4- difluorophenyl)- N-{2-fluoro-3- (1H-tetrazol-5- yl)-4-[6- (trifluoromethyl) pyridin-3- yl]phenyl}cyclopro- panecarboxa- mide | ¹H NMR (500 MHz, DMSO-d6) δ 8.89 (s, 1H), 8.50 (s, 1H), 7.90 (m, 1H), 7.85 (d, J = 8.1 Hz, 1H), 7.74 (d, J = 8.9 Hz, 1H), 7.59-7.54 (m, 1H), 7.54-7.49 (m, 1H), 7.49- 7.42 (m, 1H), 7.34 (s, 1H), 1.53 (m, 2H), 1.23 (m, 2H). LCMS (Analytical Method D) Rt = 4.40 min, MS (ESIpos): m/z = 505.25 (M + H)⁺. |
| 299 | 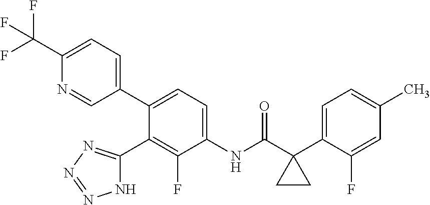 [SMILES: Cc1ccc(C2(C(=O)Nc3ccc(-c4ccc(C(F)(F)F)nc4)c(-c4nnn[nH]4)c3F)CC2)c(F)c1] | 1-(2-fluoro-4- methylphenyl)- N-{2-fluoro-3- (1H-tetrazol-5- yl)-4-[6- (trifluoromethyl) pyridin-3- yl]phenyl}cyclopro- panecarboxa- mide | ¹H NMR (500 MHz, DMSO-d6) δ 8.70 (s, 1H), 8.50 (s, 1H), 7.93 (m, 1H), 7.85 (d, J = 8.3 Hz, 1H), 7.74 (d, J = 8.2 Hz, 1H), 7.51 (d, J = 8.7 Hz, 1H), 7.39 (m, 1H), 7.08 (m, 2H), 2.35 (s, 3H), 1.57 (m, 2H), 1.17 (m, 2H). LCMS (Analytical Method D) Rt = 4.52 min, MS (ESIpos): m/z = 501.25 (M + H)⁺. |
| 300 | 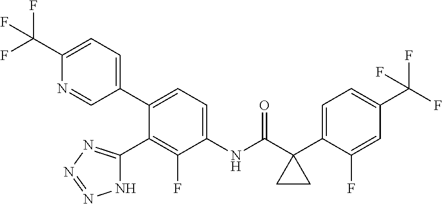 [SMILES: O=C(Nc1ccc(-c2ccc(C(F)(F)F)nc2)c(-c2nnn[nH]2)c1F)C1(c2ccc(C(F)(F)F)cc2F)CC1] | N-{2-fluoro-3- 1H-tetrazol-5- yl)-4-[6- (trifluoromethyl) pyridin-3- yl]phenyl}-1-[2- fluoro-4- (trifluoromethyl) phenyl]cyclopro- panecarboxamide | ¹H NMR (500 MHz, DMSO-d6) δ 9.10 (s, 1H), 8.51 (s, 1H), 7.85 (d, J = 8.1 Hz, 1H), 7.73 (m, 4H), 7.63 (d, J = 8.1 Hz, 1H), 7.51 (d, J = 8.3 Hz, 1H), 1.64 (m, 2H), 1.31- 1.25 (m, 2H). LCMS (Analytical Method D) Rt = 4.56 min, MS (ESIpos): m/z = 555.00 (M + H)⁺. |
| 301 | 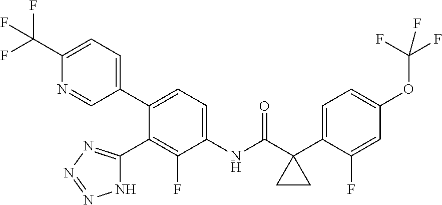 [SMILES: O=C(Nc1ccc(-c2ccc(C(F)(F)F)nc2)c(-c2nnn[nH]2)c1F)C1(c2ccc(OC(F)(F)F)cc2F)CC1] | N-{2-fluoro-3- (1H-tetrazol-5- yl)-4-[6- (trifluoromethyl) pyridin-3- yl]phenyl}-1-[2- fluoro-4- (trifluoromethoxy) phenyl]cyclo- propanecarboxa- mide | ¹H NMR (500 MHz, DMSO-d6) δ 9.02 (s, 1H), 8.51 (s, 1H), 7.85 (d, J = 8.2 Hz, 1H), 7.82-7.72 (m, 2H), 7.64 (m, 1H), 7.51 (d, J = 8.4 Hz, 1H), 7.42 (d, J = 8.6 Hz, 1H), 7.27 (d, J = 7.9 Hz, 1H), 1.61 (m, 2H), 1.24 (m, 2H). LCMS (Analytical Method D) Rt = 4.65 min, MS (ESIpos): m/z = 571.00 (M + H)⁺. |
| 302 | 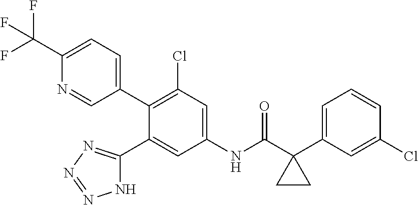 [SMILES: O=C(Nc1cc(Cl)c(-c2ccc(C(F)(F)F)nc2)c(-c2nnn[nH]2)c1)C1(c2cccc(Cl)c2)CC1] | 1-(3- chlorophenyl)- N-{3-chloro-5- (1H-tetrazol-5- yl)-4-[6- (trifluoromethyl) pyridin-3- yl]phenyl}cyclopro- panecarboxa- mide | ¹H NMR (500 MHz, DMSO-d6) δ 9.70 (s, 1H), 8.53 (s, 1H), 8.18- 8.11 (m, 2H), 7.91 (d, J = 8.1 Hz, 1H), 7.86 (d, J = 8.0 Hz, 1H), 7.46 (s, 1H), 7.43-7.35 (m, 3H), 1.56- 1.49 (m, 2H), 1.26-1.20 (m, 2H). LCMS (Analytical Method F): Rt = 4.02 min, MS (ESIpos): m/z = 519 (M + H)⁺. |
| 303 | 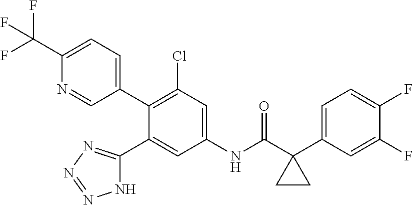 [SMILES: O=C(Nc1cc(Cl)c(-c2ccc(C(F)(F)F)nc2)c(-c2nnn[nH]2)c1)C1(c2ccc(F)c(F)c2)CC1] | N-{3-chloro-5- (1H-tetrazol-5- yl)-4-[6- (trifluoromethyl) pyridin-3- yl]phenyl}-1- (3,4- difluorophenyl)cy- clopropanecar- boxamide | ¹H NMR (500 MHz, DMSO-d6) δ 9.48 (s, 1H), 8.54 (d, J = 1.5 Hz, 1H), 8.15 (m, 2H), 7.91 (d, J = 8.0 Hz, 1H), 7.87 (dd, J = 8.1, 1.8 Hz, 1H), 7.52 (m, 1H), 7.43 (m, 1H), 7.32-7.25 (m, 1H), 1.59-1.47 (m, 2H), 1.29-1.14 (m, 2H). LCMS (Analytical Method F): Rt = 3.89 min; MS (ESIpos) = 521, 523 (M + H)⁺. |
| 304 | 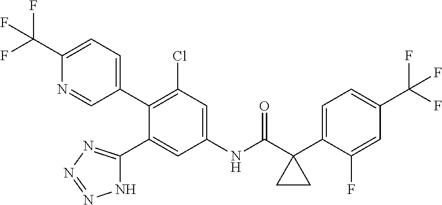 [SMILES: O=C(Nc1cc(Cl)c(-c2ccc(C(F)(F)F)nc2)c(-c2nnn[nH]2)c1)C1(c2ccc(C(F)(F)F)cc2F)CC1] | N-{3-chloro-5- (1H-tetrazol-5- yl)-4-[6- (trifluoromethyl) pyridin-3- yl]phenyl}-1-[2- fluoro-4- (trifluoromethyl) phenyl]cyclopro- panecarboxamide | ¹H NMR (500 MHz, DMSO-d6) δ 9.47 (s, 1H), 8.54 (s, 1H), 8.15 (s, 1H), 8.12 (d, J = 2.0 Hz, 1H), 7.91 (d, J = 8.1 Hz, 1H), 7.89-7.86 (m, 1H), 7.76-7.68 (m, 2H), 7.63 (d, J = 8.1 Hz, 1H), 1.72-1.65 (m, 2H), 1.34-1.27 (m, 2H). LCMS (Analytical Method F): Rt = 4.10 min; MS (ESIpos) m/z = 571 (M + H)⁺. |

In a parallel synthesis approach, 42 mg (150 μmol, 0.25 M in NMP) 4-(6-ethoxypyridin-3-yl)-3-(1H-tetrazol-5-yl)aniline, 2 equivalents of the respective carboxylic acid (0.5 M in NMP), 2 equivalents of HATU (0.5 M in NMP) and 0.2 mL N-methylmorpholine (3 M in NMP, additionally containing 2.5% DMAP) were mixed in a DWP and shaken at RT for ca. 14 h. The reaction mixture was then directly purified by preparative HPLC to yield the respective example compounds:

| Ex. | Structure | Name | Analytical Data |
|---|---|---|---|
| 306 | 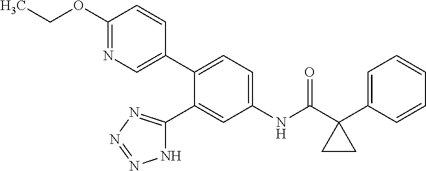 [SMILES: CCOc1ccc(-c2ccc(NC(=O)C3(c4ccccc4)CC3)cc2-c2nnn[nH]2)cn1] | N-[4-(6- ethoxypyridin-3- yl)-3-(1H- tetrazol-5- yl)phenyl]-1- phenylcyclopropane- carboxamide | LCMS (method 5): Rt = 1.29 min, m/z = 427 (M + H)⁺ |
| 309 | 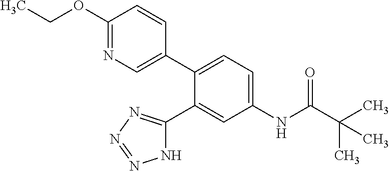 [SMILES: CCOc1ccc(-c2ccc(NC(=O)C(C)(C)C)cc2-c2nnn[nH]2)cn1] | N-[4-(6- ethoxypyridin-3- yl)-3-(1H- tetrazol-5- yl)phenyl]-2,2- dimethylpropan- amide | LCMS (method 5): Rt = 1.15 min, m/z = 367 (M + H)⁺ |

### Example 313: 1-(3-chlorophenyl)-N-{3-(1H-tetrazol-5-yl)-4-[6-(2,2,2-trifluoroethoxy)-pyridin-3-yl]phenyl}cyclopropanecarboxamide

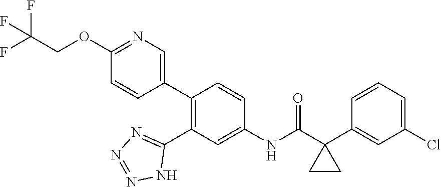 [SMILES: O=C(Nc1ccc(-c2ccc(OCC(F)(F)F)nc2)c(-c2nnn[nH]2)c1)C1(c2cccc(Cl)c2)CC1]

a) Water (0.6 mL) and 1,2-dimethoxyethane (0.74 mL) were degassed with a stream of argon for 15 mins. Then N-[4-bromo-3-(2-{[2-(trimethylsilyl)ethoxy]methyl}-2H-tetrazol-5-yl)phenyl]-1-(3-chlorophenyl)cyclopropanecarboxamide (or/and N-[4-bromo-3-(1-{[2-(trimethylsilyl)ethoxy]methyl}-1H-tetrazol-5-yl)phenyl]-1-(3-chlorophenyl)cyclopropanecarboxamide) (192 g, 0.35 mmol), [6-(2,2,2-trifluoroethoxy)pyridin-3-yl]boronic acid (77 mg, 0.35 mmol), potassium carbonate (160 mg, 1.15 mmol) and dichlorobis(triphenylphosphine) palladium(II) (3.0 mg, 4 μmol) were added, and the reaction heated at 90° C. for 2 h. The mixture was cooled and put into water. Ethyl acetate was added and the layers separated. The aqueous layer was extracted 3× with EE. The combined organic layers were washed with brine, dried with sodium sulfate and concentrated under reduced pressure.

b) The crude intermediate was re-dissolved in MeOH (3 mL) and 3M HCl (2 mL) were added. The mixture was stirred for 1 h at 60° C., then cooled to RT and evaporated to dryness under reduced pressure. The crude product was purified by preparative HPLC to give 39.7 mg (20% yield) of the deprotected Suzuki product.

¹H NMR (400 MHz, DMSO-d6) δ[ppm] 1.19-1.22 (m, 2H), 1.49-1.51 (m, 2H), 4.98 (q, 2H), 6.90 (d, 1H), 7.36-7.41 (m, 3H), 7.42-7.47 (m, 3H), 7.87 (dd, 1H), 7.94-7.96 (m, 2H), 9.54 (s, 1H).

LCMS (method 1): Rt=1.32; MS (ESipos) m/z=515 (M+H)⁺.

In analogy to the procedure described for Example 313 the following examples were prepared using the corresponding boronic acids or, respectively, the corresponding pinacol boronic esters as building blocks.

| Ex. | Structure | Name | Analytical Data |
|---|---|---|---|
| 314 | 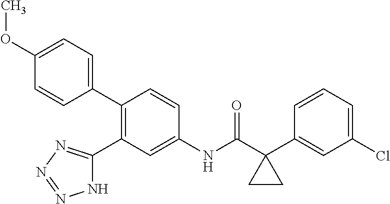 [SMILES: COc1ccc(-c2ccc(NC(=O)C3(c4cccc(Cl)c4)CC3)cc2-c2nnn[nH]2)cc1] | 1-(3- chlorophenyl)- N-[4′-methoxy- 2-(1H-tetrazol- 5-yl)biphenyl-4- yl]cyclopropane- carboxamide | ¹H NMR (400 MHz, DMSO-d6) δ [ppm] 1.18-1.21 (m, 2H), 1.48- 1.51 (m, 2H), 3.73 (s, 3H), 6.84 (d, 2H), 6.97 (d, 2H), 7.36-7.45 (m, 5H), 7.84 (s, 1H), 7.86 (d, 1H), 9.48 (s, 1H). LCMS (method 2): Rₜ = 1.22 min; MS (ESIpos) m/z = 446 (M + H)⁺ |
| 315 | 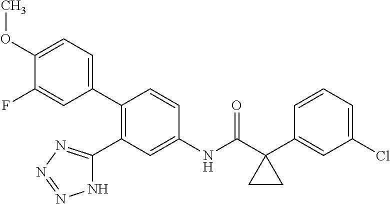 [SMILES: COc1ccc(-c2ccc(NC(=O)C3(c4cccc(Cl)c4)CC3)cc2-c2nnn[nH]2)cc1F] | 1-(3- chlorophenyl)- N-[3′-fluoro-4′- methoxy-2-(1H- tetrazol-5- yl)biphenyl-4- yl]cyclopropane- carboxamide | ¹H NMR (400 MHz, DMSO-d6) δ [ppm] 1.18-1.21 (m, 2H), 1.48- 1.51 (m, 2H), 3.82 (s, 3H), 6.73- 6.77 (m, 1H), 6.94 (dd, 1H), 7.05 (dd, 1H), 7.35-7.45 (m, 5H), 7.50 (d, 1H), 7.83 (d, 1H), 7.85 (s, 1H), 9.48 (s, 1H). LCMS (method 2): Rₜ = 1.23 min; MS (ESIpos) m/z = 464 (M + H)⁺ |
| 316 | 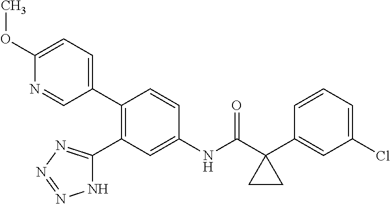 [SMILES: COc1ccc(-c2ccc(NC(=O)C3(c4cccc(Cl)c4)CC3)cc2-c2nnn[nH]2)cn1] | 1-(3- chlorophenyl)- N-[4-(6- methoxypyridin- 3-yl)-3-(1H- tetrazol-5- yl)phenyl]cyclopro- panecarboxa- mide | ¹H NMR (400 MHz, DMSO-d6) δ [ppm] 1.20-1.22 (m, 2H), 1.49- 1.52 (m, 2H), 3.85 (s, 3H), 6.75 (d, 1H), 7.33 (dd, 3H), 7.36- 7.42 (m, 3H), 7.45-7.46 (m, 1H), 7.49 (d, 1H), 7.90 (dd, 1H), 7.92 (dd, 1H), 7.96 (d, 1H), 9.55 (s, 1H). LCMS (method 2): Rₜ = 1.15 min; MS (ESIpos) m/z = 447 (M + H)⁺ |
| 317 | 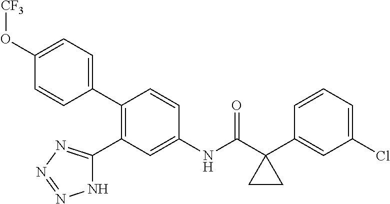 [SMILES: O=C(Nc1ccc(-c2ccc(OC(F)(F)F)cc2)c(-c2nnn[nH]2)c1)C1(c2cccc(Cl)c2)CC1] | 1-(3- chlorophenyl)- N-[2-(1H- tetrazol-5-yl)- 4′- (trifluoromethoxy) biphenyl-4- yl]cyclopropane- carboxamide | ¹H NMR (400 MHz, DMSO-d6) δ [ppm] 1.21 (s, br, 2H), 1.50 (s, br, 2H), 7.17 (d, 2H), 7.30 (d, 2H), 7.36-7.41 (m, 3H), 7.45 (s, br, 1H), 7.51 (d, 1H), 7.90-7.95 (m, 2H), 9.56 (s, 1H). LCMS (method 2): Rₜ = 1.35 min; MS (ESIpos) m/z = 500 (M + H)⁺ |
| 318 | 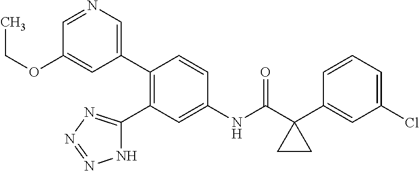 [SMILES: CCOc1cncc(-c2ccc(NC(=O)C3(c4cccc(Cl)c4)CC3)cc2-c2nnn[nH]2)c1] | 1-(3- chlorophenyl)- N-[4-(5- ethoxypyridin-3- yl)-3-(1H- tetrazol-5- yl)phenyl]cyclopro- panecarboxa- mide | ¹H NMR (400 MHz, DMSO-d6) δ [ppm] 1.21 (s, br, 2H), 1.29 (t, 3H), 1.51 (m, 2H), 4.00 (q, 2H), 7.02 (s, 1H), 7.39 (s, br, 3H), 7.45 (s, br, 1H), 7.56 (d, 1H), 7.82 (s, 1H), 7.93 (d, 1H), 7.99 (s, 1H), 8.19 (s, 1H), 9.58 (s, 1H). LCMS (method 2): Rₜ = 1.06 min; MS (ESIpos) m/z = 461 (M + H)⁺. |
| 319 | 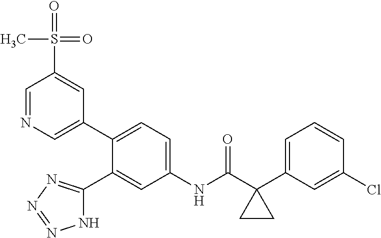 [SMILES: CS(=O)(=O)c1cncc(-c2ccc(NC(=O)C3(c4cccc(Cl)c4)CC3)cc2-c2nnn[nH]2)c1] | 1-(3- Chlorophenyl)- N-{4-[5- (methylsulfonyl) pyridin-3-yl]-3- (1H-tetrazol-5- yl)phenyl}cyclopro- panecarboxa- mide | ¹H NMR (400 MHz, DMSO-d6) δ [ppm] 1.20-1.23 (m, 2H), 1.51- 1.53 (m, 2H), 3.29 (s, 3H), 7.38- 7.41 (m, 3H), 7.46-7.47 (m, 1H), 7.61 (d, 1H), 7.94 (dd, 1H), 8.01 (t, 1H), 8.10 (d, 1H), 8.59 (d, 1H), 8.97 (d, 1H), 9.61 (s, 1H). LCMS (method 1): Rt = 1.05 min; MS (ESIpos) m/z = 495 (M + H)⁺. |
| 320 | 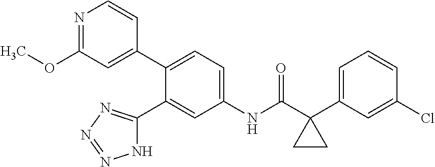 | 1-(3- Chlorophenyl)- N-[4-(2- methoxypyridin- 4-yl)-3-(1H- tetrazol-5- yl)phenyl]cyclopro- panecarboxa- mide | ¹H NMR (400 MHz, DMSO-d6) δ [ppm] 1.21-1.23 (m, 2H), 1.51- 1.53 (m, 2H), 3.84 (s, 3H), 6.54 (d, 1H), 6.57 (dd, 1H), 7.37- 7.46 (m, 4H), 7.54 (d, 1H), 7.93 (dd, 1H), 7.98 (d, 1H), 8.05 (d, 1H), 9.58 (s, 1H). LCMS (method 1): Rt = 1.25 min; MS (ESIpos) m/z = 477 (M + H)⁺. |
| 321 | 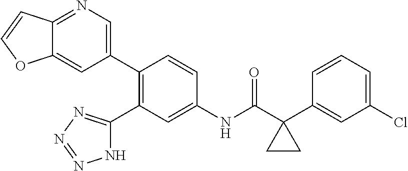 [SMILES: O=C(Nc1ccc(-c2cnc3ccoc3c2)c(-c2nnn[nH]2)c1)C1(c2cccc(Cl)c2)CC1] | 1-(3- chlorophenyl)- N-[4-(furo[3,2- b]pyridin-6-yl)- 3-(1H-tetrazol- 5- yl)phenyl]cyclopro- panecarboxa- mide | ¹H NMR (400 MHz, DMSO-d6) δ [ppm] 1.22 (s, br, 2H), 1.52 (s, br, 2H), 7.15 (s, br, 1H), 7.40 (s, br, 3H), 7.46 (s, br, 1H), 7.59 (d, br, 1H), 7.82 (s, br, 1H), 7.94 (d, br, 1H), 8.04 (s, br, 1H), 8.15 (s, br, 1H), 8.33 (s, br, 1H), 9.59 (s, 1H). LCMS (method 2): Rₜ = 1.08 min; MS (ESIpos) m/z = 457 (M + H)⁺ |
| 322 | 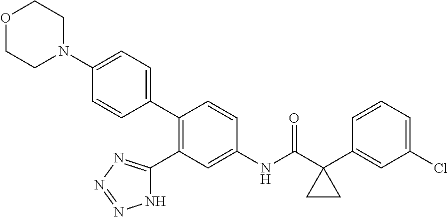 [SMILES: O=C(Nc1ccc(-c2ccc(N3CCOCC3)cc2)c(-c2nnn[nH]2)c1)C1(c2cccc(Cl)c2)CC1] | 1-(3- chlorophenyl)- N-[4′- (morpholin-4- yl)-2-(1H- tetrazol-5- yl)biphenyl-4- yl]cyclopropane carboxamide | ¹H NMR (400 MHz, DMSO-d6) δ [ppm] 1.19 (s, br, 2H), 1.49 (s, br, 2H), 3.10 (s, br, 4H), 3.72 (s, br, 4H), 6.85 (d, 2H), 6.89 (d, 2H), 7.38 (s, br, 3H), 7.44 (s, br, 2H), 7.83-7.86 (m, 2H), 9.47 (s, 1H). LCMS (method 2): Rₜ = 1.18 min; MS (ESIpos) m/z = 501 (M + H)⁺ |
| 323 | 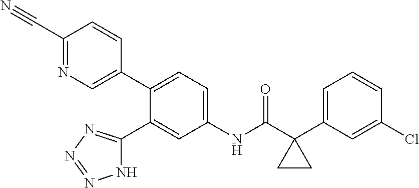 [SMILES: N#Cc1ccc(-c2ccc(NC(=O)C3(c4cccc(Cl)c4)CC3)cc2-c2nnn[nH]2)cn1] | 1-(3- chlorophenyl)- N-[4-(6- cyanopyridin-3- yl)-3-(1H- tetrazol-5- yl)phenyl]cyclopro- panecarboxa- mide | ¹H NMR (400 MHz, DMSO-d6) δ [ppm] 1.21-1.24 (m, 2H), 1.50- 1.53 (m, 2H), 7.36-7.40 (m, 3H), 7.45-7.46 (m, 1H), 7.59 (d, 1H), 7.77 (d, 1H), 7.96 (dd, 1H), 8.01 (dd, 1H), 8.12 (d, 1H), 8.50 (dd, 1H), 9.66 (s, 1H). LCMS (method 1): Rt = 1.14 min; MS (ESIpos) m/z = 442 (M + H)⁺ |
| 325 | 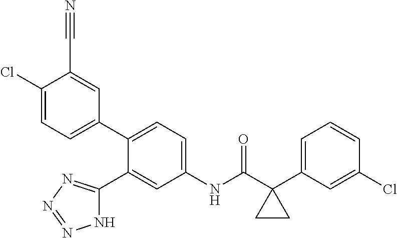 [SMILES: N#Cc1cc(-c2ccc(NC(=O)C3(c4cccc(Cl)c4)CC3)cc2-c2nnn[nH]2)ccc1Cl] | N-[4′-chloro-3′- cyano-2-(1H- tetrazol-5- yl)biphenyl-4- yl]-1-(3- chlorophenyl)cy- clopropanecarbox- amide | ¹H NMR (400 MHz, DMSO-d6) δ [ppm] 1.19-1.24 (m, 2H), 1.50- 1.52 (m, 2H), 7.31 (dd, 1H), 7.37- 7.41 (m, 3H), 7.45-7.46 (m, 1H), 7.53 (d, 1H), 7.66 (d, 1H), 7.84 (d, 1H), 7.91 (dd, 1H), 8.05 (s, br, 1H), 9.60 (s, 1H). LCMS (method 1): Rt = 1.28 min; MS (ESIpos) m/z = 475 (M + H)⁺ |
| 326 | 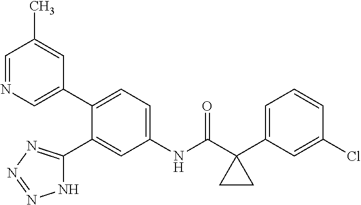 [SMILES: Cc1cncc(-c2ccc(NC(=O)C3(c4cccc(Cl)c4)CC3)cc2-c2nnn[nH]2)c1] | 1-(3- chlorophenyl)- N-[4-(5- methylpyridin- 3-yl)-3-(1H- tetrazol-5- yl)phenyl]cyclopro- panecarboxa- mide | ¹H NMR (400 MHz, DMSO-d6) δ [ppm] 1.20-1.24 (m, 2H), 1.49- 1.52 (m, 2H), 2.25 (s, 3H), 7.34- 7.41 (m, 4H), 7.45-7.46 (m, 1H), 7.52 (d, 1H), 7.92 (dd, 1H), 8.00 (dd, 1H), 8.33 (d, 1H), 9.57 (s, 1H). LCMS (method 1): Rt = 1.02 min; MS (ESIpos) m/z = 431 (M + H)⁺ |
| 327 | 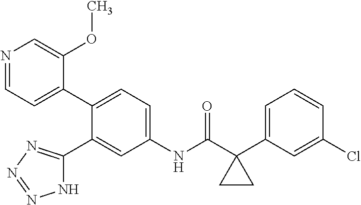 [SMILES: COc1cnccc1-c1ccc(NC(=O)C2(c3cccc(Cl)c3)CC2)cc1-c1nnn[nH]1] | 1-(3- chlorophenyl)- N-[4-(3- methoxypyridin- 4-yl)-3-(1H- tetrazol-5- yl)phenyl]cyclopro- panecarboxa- mide | ¹H NMR (400 MHz, DMSO-d6) δ [ppm] 1.20-1.23 (m, 2H), 1.49- 1.52 (m, 2H), 3.40 (s, 3H), 7.25 (d, 1H), 7.37-7.40 (m, 3H), 7.42 (d, 1H), 7.45-7.46 (m, 1H), 7.88 (dd, 1H), 8.04 (d, 1H), 8.23 (s, 1H), 8.26 (d, 1H), 9.60 (s, 1H). LCMS (method 1): Rₜ = 0.91 min; MS (ESIpos) m/z = 447 (M + H)⁺ |
| 328 | 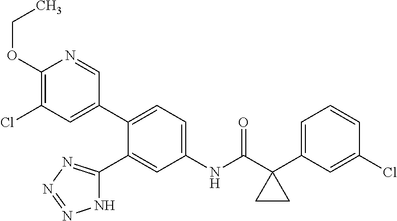 [SMILES: CCOc1ncc(-c2ccc(NC(=O)C3(c4cccc(Cl)c4)CC3)cc2-c2nnn[nH]2)cc1Cl] | N-[4-(5-chloro- 6-ethoxypyridin- 3-yl)-3-(1H- tetrazol-5- yl)phenyl]-1-(3- chlorophenyl)cy- clopropanecarbox- amide | ¹H NMR (400 MHz, DMSO-d6) δ [ppm] 1.20-1.23 (m, 2H), 1.34 (t, 3H), 1.49-1.52 (m, 2H), 4.38 (q, 2H), 7.36-7.41 (m, 3H), 7.44- 7.45 (m, 1H), 7.52 (d, 1H), 7.62 (d, 1H), 7.78 (d, 1H), 7.90 (dd, 1H), 8.02 (s, br, 1H), 9.58 (s, 1H). LCMS (method 1): Rₜ = 1.36 min; MS (ESIpos) m/z = 495 (M + H)⁺ |
| 329 | 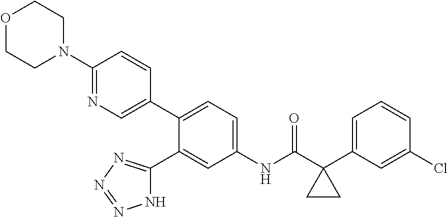 [SMILES: O=C(Nc1ccc(-c2ccc(N3CCOCC3)nc2)c(-c2nnn[nH]2)c1)C1(c2cccc(Cl)c2)CC1] | 1-(3- chlorophenyl)- N-{4-[6- (morpholin-4- yl)pyridin-3-yl]- 3-(1H-tetrazol- 5- yl)phenyl}cyclopro- panecarboxa- mide | ¹H NMR (400 MHz, DMSO-d6) δ [ppm] 1.19-1.22 (m, 2H), 1.48- 1.51 (m, 2H), 3.43-3.46 (m, 4H), 3.67-3.70 (m, 4H), 6.76 (d, 1H), 7.17 (d, 1H), 7.37-7.40 (m, 3H), 7.44-7.45 (m, 1H), 7.47 (d, 1H), 7.86-7.89 (m, 2H), 7.91 (s, br, 1H), 9.52 (s, 1H). LCMS (method 1): Rₜ = 0.99 min; MS (ESIpos) m/z = 502 (M + H)⁺ |
| 330 | 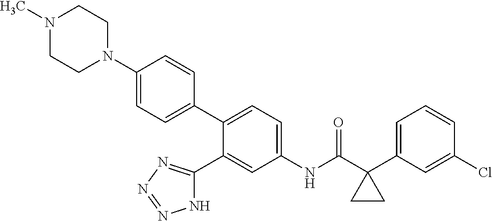 [SMILES: CN1CCN(c2ccc(-c3ccc(NC(=O)C4(c5cccc(Cl)c5)CC4)cc3-c3nnn[nH]3)cc2)CC1] | 1-(3- chlorophenyl)- N-[4′-(4- methylpiperazin- 1-yl)-2-(1H- tetrazol-5- yl)biphenyl-4- yl]cyclopropane carboxamide | ¹H NMR (400 MHz, DMSO-d6) δ [ppm] 1.17-1.20 (m, 2H), 1.47- 1.50 (m, 2H), 2.55 (s, 3H), 2.87 (s, br, 4H), 3.26 (s, br, 4H), 6.86 (d, 2H), 6.91 (d, 2H), 7.35-7.42 (m, 4H), 7.44-7.45 (m, 1H), 7.79 (d, 1H), 7.83 (dd, 1H), 7.91 (s, br, 1H), 9.45 (s, 1H). LCMS (method 1): Rₜ = 0.87 min; MS (ESIpos) m/z = 514 (M + H)⁺ |
| 331 | 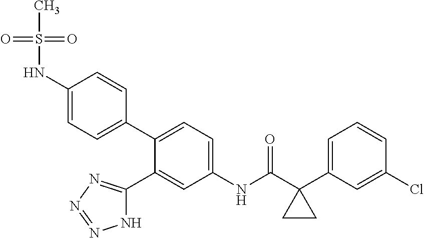 [SMILES: CS(=O)(=O)Nc1ccc(-c2ccc(NC(=O)C3(c4cccc(Cl)c4)CC3)cc2-c2nnn[nH]2)cc1] | 1-(3- chloropehnyl)- N-{4′- [(methylsulfonyl) amino]-2-(1H- tetrazol-5- yl)biphenyl-4- yl}cyclopropane carboxamide | ¹H NMR (400 MHz, DMSO-d6) δ [ppm] 1.19-1.21 (m, 2H), 1.48- 1.51 (m, 2H), 3.00 (s, 3H), 7.00 (d, 2H), 7.11 (d, 2H), 7.35-7.40 (m, 3H), 7.44-7.48 (m, 2H), 7.62-7.65 (m, 1H), 7.89 (dd, 1H), 9.52 (s, 1H), 9.83 (s, 1H). LCMS (method 1): Rₜ = 1.11 min; MS (ESIpos) m/z = 509 (M + H)⁺ |
| 332 | 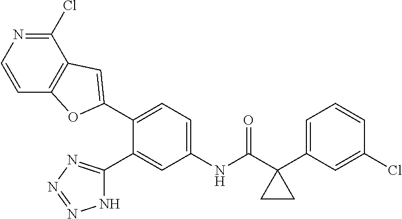 [SMILES: O=C(Nc1ccc(-c2cc3c(Cl)nccc3o2)c(-c2nnn[nH]2)c1)C1(c2cccc(Cl)c2)CC1] | N-[4-(4- chlorofuro[3,2- c]pyridin-2-yl)- 3-(1H-tetrazol- 5-yl)phenyl]-1- (3- chlorophenyl)cy- clopropanecarbox- amide | ¹H NMR (400 MHz, DMSO-d6) δ [ppm] 1.21-1.25 (m, 2H), 1.51- 1.54 (m, 2H), 6.92 (s, 1H), 7.36- 7.41 (m, 4H), 7.45-7.46 (m, 1H), 7.56 (d, 1H), 8.01-8.03 (m, 2H), 8.25 (d, 1H), 9.67 (s, 1H). LCMS (method 1): Rₜ = 1.35 min; MS (ESIpos) m/z = 491 (M + H)⁺ |
| 333 | 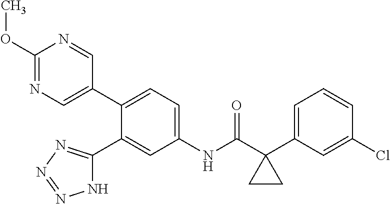 [SMILES: COc1ncc(-c2ccc(NC(=O)C3(c4cccc(Cl)c4)CC3)cc2-c2nnn[nH]2)cn1] | 1-(3- chlorophenyl)- N-[4-(2- methoxypyrimidin- 5-yl)-3-(1H- tetrazol-5- yl)phenyl]cyclopro- panecarboxa- mide | ¹H NMR (400 MHz, DMSO-d6) δ [ppm] 1.19-1.22 (m, 2H), 1.49- 1.52 (m, 2H), 3.93 (d, 3H), 7.37- 7.41 (m, 3H), 7.45-7.46 (m, 1H), 7.48 (d, 1H), 7.88 (dd, 1H), 8.06 (d, 1H), 8.34 (s, 2H), 9.57 (s, 1H). LCMS (method 1): Rₜ = 1.09 min; MS (ESIpos) m/z = 448 (M + H)⁺ |
| 334 | 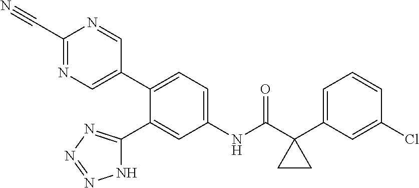 [SMILES: N#Cc1ncc(-c2ccc(NC(=O)C3(c4cccc(Cl)c4)CC3)cc2-c2nnn[nH]2)cn1] | 1-(3- chlorophenyl)- N-[4-(2- cyanopyrimidin- 5-yl)-3-(1H- tetrazol-5- yl)phenyl]cyclopro- panecarboxa- mide | ¹H NMR (400 MHz, DMSO-d6) δ [ppm] 1.22-1.25 (m, 2H), 1.51- 1.54 (m, 2H), 7.37-7.41 (m, 3H), 7.45-7.46 (m, 1H), 7.63 (d, 1H), 7.97 (dd, 1H), 8.24 (s, 1H), 8.86 (s, 2H), 9.70 (s, 1H). LCMS (method 1): Rₜ = 1.15 min; MS (ESIpos) m/z = 443 (M + H)⁺ |
| 335 | 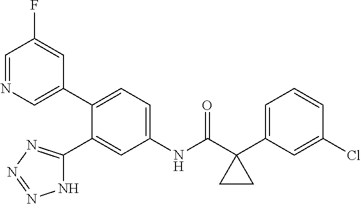 [SMILES: O=C(Nc1ccc(-c2cncc(F)c2)c(-c2nnn[nH]2)c1)C1(c2cccc(Cl)c2)CC1] | 1-(3- chlorophenyl)- N-[4-(5- fluoropyridin-3- yl)-3-(1H- tetrazol-5- yl)phenyl]cyclopro- panecarboxa- mide | ¹H NMR (400 MHz, DMSO-d6) δ [ppm] 1.21-1.23 (m, 2H), 1.50- 1.53 (m, 2H), 7.36-7.41 (m, 3H), 7.45-7.46 (m, 1H), 7.53- 7.58 (m, 2H), 7.94 (dd, 1H), 8.06- 8.10 (m, 2H), 8.52 (d, 1H), 9.62 (s, 1H). LCMS (method 1): Rₜ = 1.12 min; MS (ESIpos) m/z = 435 (M + H)⁺ |
| 336 | 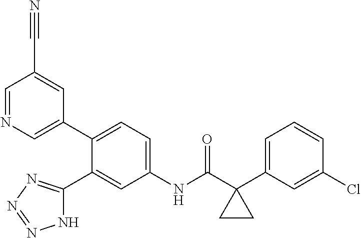 [SMILES: N#Cc1cncc(-c2ccc(NC(=O)C3(c4cccc(Cl)c4)CC3)cc2-c2nnn[nH]2)c1] | 1-(3- chlorophenyl)- N-[4-(5- cyanopyridin-3- yl)-3-(1H- tetrazol-5- yl)phenyl]cyclopro- panecarboxa- mide | ¹H NMR (400 MHz, DMSO-d6) δ [ppm] 1.21-1.24 (m, 2H), 1.50- 1.53 (m, 2H), 7.38-7.40 (m, 3H), 7.45-7.46 (m, 1H), 7.57 (d, 1H), 7.94 (dd, 1H), 8.13 (s, 1H), 8.20 (t, 1H), 8.52 (d, 1H), 8.97 (d, 1H), 9.64 (s, 1H). LCMS (method 1): Rₜ = 1.23 min; MS (ESIpos) m/z = 442 (M + H)⁺ |
| 337 | 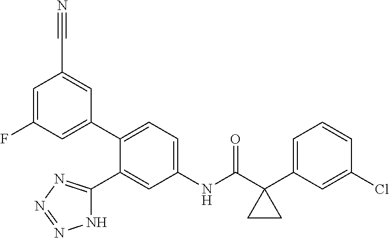 [SMILES: N#Cc1cc(F)cc(-c2ccc(NC(=O)C3(c4cccc(Cl)c4)CC3)cc2-c2nnn[nH]2)c1] | 1-(3- chlorophenyl)- N-[3′-cyano-5′- fluoro-2-(1H- tetrazol-5- yl)biphenyl-4- yl]cyclopropane carboxamide | ¹H NMR (400 MHz, DMSO-d6) δ [ppm] 1.20-1.24 (m, 2H), 1.49- 1.52 (m, 2H), 7.29-7.32 (m, 1H), 7.37-7.41 (m, 3H), 7.44- 7.46 (m, 2H), 7.51 (d, 1H), 7.80 (d, 1H), 7.89 (dd, 1H), 8.02 (d, 1H), 9.58 (s, 1H). LCMS (method 1): Rₜ = 1.24 min; MS (EISpos) m/z = 459 (M + H)⁺ |
| 338 | 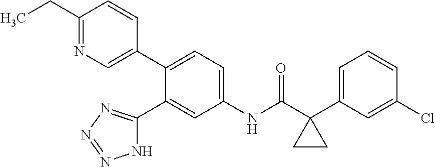 [SMILES: CCc1ccc(-c2ccc(NC(=O)C3(c4cccc(Cl)c4)CC3)cc2-c2nnn[nH]2)cn1] | 1-(3- chlorophenyl)- N-[4-(6- ethylpyridin-3- yl)-3-(1H- tetrazol-5- yl)phenyl]cyclopro- panecarboxa- mide | ¹H NMR (500 MHz, DMOS-d6) δ [ppm] 9.51 (s, 1H), 8.19 (d, J = 2.2 Hz, 1H), 7.93 (d, J = 2.1 Hz, 1H), 7.87 (dd, J = 8.5, 2.2 Hz, 1H), 7.49-7.42 (m, 2H), 7.42- 7.28 (m, 4H), 7.16 (d, J = 8.1 Hz, 1H), 2.72 (q, J = 7.6 Hz, 2H), 1.52-1.45 (m, 2H), 1.27-1.17 (m, 5H) LCMS (Analytical Method F): Rt = 2.46 mins; MS (ESIpos) m/z = 445 |
| | | | (M + H)⁺. |
| 339 | 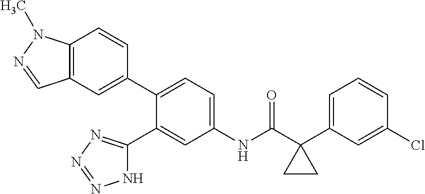 [SMILES: Cn1ncc2cc(-c3ccc(NC(=O)C4(c5cccc(Cl)c5)CC4)cc3-c3nnn[nH]3)ccc21] | 1-(3- chlorophenyl)- N-[4-(1-methyl- 1H-indazol-5- yl)-3-(1H- tetrazol-5- yll)phenyl]cyclopro- panecarboxa- mide | 1H NMR (500 MHz, DMSO-d6) δ [ppm] 9.61 (s, 1H), 8.16 (s, 1H), 8.04-7.97 (m, 2H), 7.68 (s, 1H), 7.65-7.50 (m, 6H), 7.18-7.12 (m, 1H), 4.18 (s, 3H), 1.70-1.64 (m, 2H), 1.39-1.32 (m, 2H). LCMS (Analytical Method F): Rt = 3.39 mins; MS (ESIpos) m/z = 470 (M + H)⁺ |
| 340 | 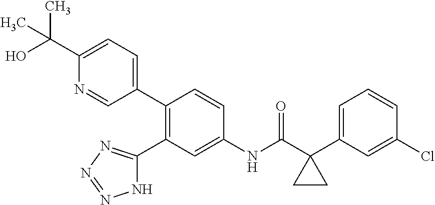 [SMILES: CC(C)(O)c1ccc(-c2ccc(NC(=O)C3(c4cccc(Cl)c4)CC3)cc2-c2nnn[nH]2)cn1] | 1-(3- chlorophenyl)- N-{4-[6-(2- hydroxypropan- 2-yl)pyridin-3- yl]-3-(1H- tetrazol-5- yl)phenyl}cyclopro- panecarboxa- mide | ¹H NMR (500 MHz, Methanol-d4) δ [ppm] 8.25-8.22 (m, 1H), 7.91- 7.87 (m, 1H), 7.84-7.79 (m, 1H), 7.70-7.64 (m, 1H), 7.64- 7.59 (m, 1H), 7.54-7.48 (m, 2H), 7.46-7.33 (m, 3H), 1.66- 1.58 (m, 2H), 1.54 (s, 6H), 1.26- 1.20 (m, 2H) LCMS (Analytical Method F): Rt = 2.55 mins; MS (ESIpos) m/z = 475 (M + H)⁺. |
| 341 | 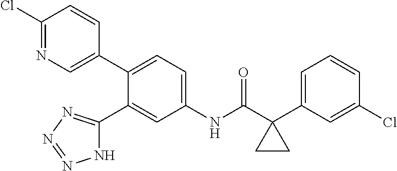 [SMILES: O=C(Nc1ccc(-c2ccc(Cl)nc2)c(-c2nnn[nH]2)c1)C1(c2cccc(Cl)c2)CC1] | 1-(3- chlorophenyl)- N-[4-(6- chloropyridin-3- yl)-3-(1H- tetrazol-5- yl)phenyl]cyclopro- panecarboxa- mide | ¹H NMR (400 MHz, DMSO-d6) δ = 1.20-1.24 (m, 2H), 1.49-1.52 (m, 2H), 7.35-7.55 (m, 7H), 7.92 (dd, 1H), 8.06 (s, br, 1H), 8.17 (d, 1H), 9.61 (s, 1H). LCMS (method 1): Rt = 1.18 min, m/z = 451 (M + H)⁺ |
| 342 | 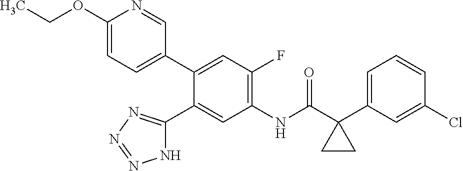 [SMILES: CCOc1ccc(-c2cc(F)c(NC(=O)C3(c4cccc(Cl)c4)CC3)cc2-c2nnn[nH]2)cn1] | 1-(3- chlorophenyl)- N-[4-(6- ethoxypyridin-3- yl)-2-fluoro-5- (1H-tetrazol-5- yl)phenyl]cyclopro- panecarboxa- mide | ¹H NMR (400 MHz, DMSO-d6) δ [ppm] 1.21-1.24 (m, 2H), 1.31 (t, 3H), 1.50-1.52 (m, 2H), 4.29 (q, 2H), 6.72 (d, 1H), 7.34 (dd, 1H), 7.41-7.49 (m, 3H), 7.52 (d, 1H), 7.65-7.67 (m, 1H), 7.90 (d, 1H), 7.94 (dd, 1H), 8.94 (s, 1H). LCMS (method 1): Rt = 1.29 min; MS (ESIpos) m/z = 479 (M + H)⁺. |

### Example 343: 1-(3-Chlorophenyl)-N-{4-[6-(2-hydroxy-2-methylpropyl)pyridin-3-yl]-3-(1H-tetrazol-5-yl) phenyl}cyctopropanecarboxamide

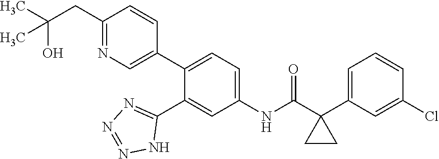 [SMILES: CC(C)(O)Cc1ccc(-c2ccc(NC(=O)C3(c4cccc(Cl)c4)CC3)cc2-c2nnn[nH]2)cn1]

Intermediate 239A (110 mg, 0.159 mmol, as a 6:4 mixture of SEM protected regioisomers) was dissolved in THF (5 mL) and tetra-n-butylammonium fluoride (0.47 mL of a 1M solution in THF, 0.47 mmol) was added and the resulting mixture was heated at 65° C. for 5 hours.

The mixture was diluted with EtOAc (50 mL) and washed with brine (3×20 mL), dried (Na₂SO₄), filtered and concentrated at reduced pressure. The residue was purified by preparative HPLC (Method 1) to give 44 mg (56% yield) of the title compound as a white powder.

¹H NMR (500 MHz, DMSO-d6) δ[ppm] 9.52 (s, 1H), 8.18 (d, J=2.0 Hz, 1H), 7.95 (d, J=1.9 Hz, 1H), 7.89 (dd, J=8.5, 2.1 Hz, 1H), 7.49 (d, J=8.5 Hz, 1H), 7.45 (d, J=1.6 Hz, 1H), 7.43-7.30 (m, 4H), 7.21 (d, J=8.0 Hz, 1H), 2.81 (s, 2H), 1.54-1.47 (m, 2H), 1.23-1.17 (m, 2H), 1.08 (s, 6H).

LCMS (Analytical Method F): Rt=2.38 mins; MS (ESipos) m/z=489 (M+H)⁺.

### Example 345: 1-[2-Fluoro-4-(trifluoromethyl)phenyl]-N-{3-(1H-tetrazol-5-yl)-4-[4-(trifluoromethyl)-1H-pyrazol-1-yl]phenyl}cyclopropanecarboxamide

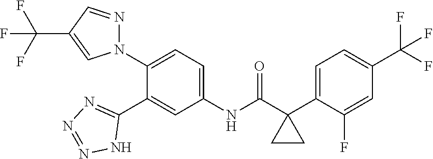 [SMILES: O=C(Nc1ccc(-n2cc(C(F)(F)F)cn2)c(-c2nnn[nH]2)c1)C1(c2ccc(C(F)(F)F)cc2F)CC1]

To a solution of 3-(1H-tetrazol-5-yl)-4-[4-(trifluoromethyl)-1H-pyrazol-1-yl]aniline (60 mg, 0.203 mmol) and 4-dimethylaminopyridine (10 mg, 0.082 mmol) in pyridine (1 mL) was added a solution of 1-[2-fluoro-4-(trifluoromethyl)phenyl]cyclopropanecarbonyl chloride (81 mg, 0.305 mmol) in DCM (0.5 mL) at RT. The mixture was stirred for 1 hour at this temperature.

The volatiles were removed at reduced pressure and the residue was purified by preparative HPLC (Method A). The product containing fractions were combined, concentrated to a small volume (˜5 ml) and acidified with conc. HCL. The resulting precipitate was collected by filtration, washed with water (˜5 ml) and dried in the vacuum oven at 40° C. for 4 hours to afford 79 mg (73%) of the title compound as a white solid.

¹H NMR (500 MHz, DMSO-d6) δ[ppm] 9.40 (s, 1H), 8.74 (s, 1H), 8.07 (s, 1H), 7.96 (s, 1H), 7.94 (dd, J=8.8, 2.3 Hz, 1H), 7.75-7.59 (m, 4H), 1.74-1.61 (m, 2H), 1.34-1.21 (m, 2H).

LCMS (Analytical Method F): Rt=3.75 mins; MS (ESipos) m/z=526.1 (M+H)⁺.

In analogy to the procedure described for Example 345, the following examples were prepared:

| Ex. | Structure | Name | Analytical Data |
|---|---|---|---|
| 346 | 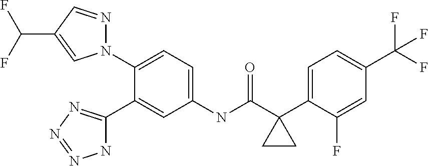 [SMILES: O=C(Nc1ccc(-n2cc(C(F)F)cn2)c(-c2nnn[nH]2)c1)C1(c2ccc(C(F)(F)F)cc2F)CC1] | N-{4-[4- (Difluoromethyl)- 1H-pyrazol-1- yl]-3-(1H- tetrazol-5- yl)phenyl}-1-[2- fluoro-4- (trifluoromethyl) phenyl]cyclopro- panecarboxamide | ¹H NMR (500 MHz, DMSO-d6) δ [ppm] 9.36 (s, 1H), 8.33 (s, 1H), 8.01 (s, 1H), 7.93 (dd, J = 8.8, 2.4 Hz, 1H), 7.75 (s, 1H), 7.74- 7.67 (m, 2H), 7.66-7.59 (m, 2H), 7.04 (t, J = 55.9 Hz, 1H), 1.72-1.62 (m, 2H), 1.34-1.22 (m, 2H). LCMS (Analytical Method F): Rt = 3.51 mins; MS (ESIpos) m/z = 508.1 (M + H)⁺. |
| 347 | 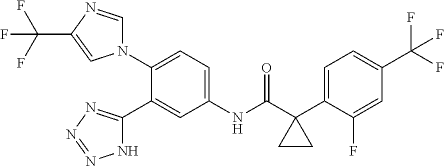 [SMILES: O=C(Nc1ccc(-n2cnc(C(F)(F)F)c2)c(-c2nnn[nH]2)c1)C1(c2ccc(C(F)(F)F)cc2F)CC1] | 1-[2-Fluoro-4- (trifluoromethyl) phenyl]-N-{3- (1H-tetrazol-5- yl)-4-[4- (trifluoromethyl)- 1H-imidazol-1- yl]phenyl}cyclopro- panecarboxa- mide | ¹H NMR (500 MHz, DMSO-d6) δ [ppm] 9.44 (s, 1H), 8.22 (s, 1H), 7.93-7.88 (m, 2H), 7.87 (s, 1H), 7.75-7.68 (m, 2H), 7.65-7.61 (m, 1H), 7.59 (d, J = 8.7 Hz, 1H), 1.74-1.61 (m, 2H), 1.35-1.21 (m, 2H). LCMS (Analytical Method F): Rt = 3.58 mins; MS (ESIpos) m/z = 526.1 (M + H)⁺. |
| 348 | 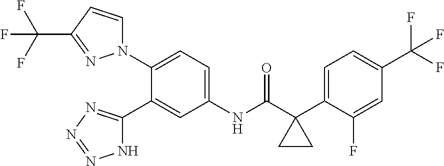 [SMILES: O=C(Nc1ccc(-n2ccc(C(F)(F)F)n2)c(-c2nnn[nH]2)c1)C1(c2ccc(C(F)(F)F)cc2F)CC1] | 1-[2-Fluoro-4- (trifluoromethyl) phenyl]-N-{3- (1H-tetrazol-5- yl)-4-[3- (trifluoromethyl)- 1H-pyrazol-1- yl]phenyl}cyclopro- panecarboxa- mide | ¹H NMR (500 MHz, DMSO-d6) δ [ppm] 9.40 (s, 1H), 8.15 (s, 1H), 8.09 (s, 1H), 7.95 (dd, J = 8.8, 2.4 Hz, 1H), 7.75-7.66 (m, 3H), 7.62 (d, J = 8.0 Hz, 1H), 6.88 (d, J = 2.5 Hz, 1H), 1.73-1.62 (m, 2H), 1.34-1.21 (m, 2H). LCMS (Analytical Method F): Rt = 3.75 mins; MS (ESIpos) m/z = 526.1 (M + H)+. |
| 349 | 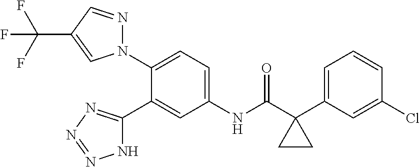 [SMILES: O=C(Nc1ccc(-n2cc(C(F)(F)F)cn2)c(-c2nnn[nH]2)c1)C1(c2cccc(Cl)c2)CC1] | 1-(3- Chlorophenyl)- N-{3-(1H- tetrazol-5-yl)-4- [4- (trifluoromethyl)- 1H-pyrazol-1- yl]phenyl}cyclopro- panecarboxa- mide | ¹H NMR (500 MHz, DMSO-d6) δ [ppm] 9.64 (s, 1H), 8.72 (s, 1H), 8.13 (s, 1H), 8.02-7.91 (m, 2H), 7.66 (d, J = 8.8 Hz, 1H), 7.45 (s, 1H), 7.42-7.34 (m, 3H), 1.58- 1.46 (m, 2H), 1.27-1.16 (m, 2H). LCMS (Analytical Method F): Rt = 3.62 mins; MS (ESIpos) m/z = 474.0 (M + H)⁺. |
| 350 | 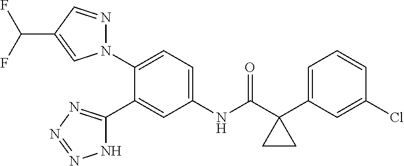 [SMILES: O=C(Nc1ccc(-n2cc(C(F)F)cn2)c(-c2nnn[nH]2)c1)C1(c2cccc(Cl)c2)CC1] | 1-(3- Chlorophenyl)- N-{4-[4- (difluoromethyl)- 1H-pyrazol-1- yl]-3-(1H- tetrazol-5- yl)phenyl}cyclopro- panecarboxa- mide | ¹H NMR (500 MHz, DMSO-d6) δ [ppm] 9.61 (s, 1H), 8.32 (s, 1H), 8.06 (s, 1H), 7.95 (dd, J = 8.8, 2.4 Hz, 1H), 7.75 (s, 1H), 7.64 (d, J = 8.8 Hz, 1H), 7.48-7.43 (m, 1H), 7.43-7.33 (m, 3H), 7.04 (t, J = 55.9 Hz, 1H), 1.58- 1.44 (m, 2H), 1.27-1.14 (m, 2H). LCMS (Analytical Method F): Rt = 3.36 mins; MS (ESIpos) m/z = |
| | | | 456.1 (M + H)⁺. |
| 351 | 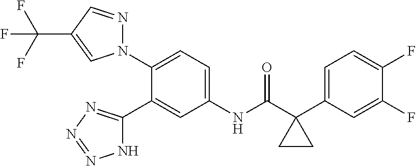 [SMILES: O=C(Nc1ccc(-n2cc(C(F)(F)F)cn2)c(-c2nnn[nH]2)c1)C1(c2ccc(F)c(F)c2)CC1] | 1-(3,4- Difluorophenyl)- N-{3-(1H- tetrazol-5-yl)-4- [4- (trifluoromethyl)- 1H-pyrazol-1- yl]phenyl}cyclopro- panecarboxa- mide | ¹H NMR (500 MHz, DMSO-d6) δ [ppm] 9.43 (s, 1H), 8.73 (s, 1H), 8.10 (s, 1H), 8.02-7.90 (m, 2H), 7.66 (d, J = 8.8 Hz, 1H), 7.52 (ddd, J = 11.7, 7.8, 2.1 Hz, 1H), 7.46-7.38 (m, 1H), 7.31-7.26 (m, 1H), 1.58-1.46 (m, 2H), 1.26-1.14 (m, 2H). LCMS (Analytical Method F): Rt = 3.50 mins; MS (ESIpos) m/z = 476.1 (M + H)⁺. |
| 352 | 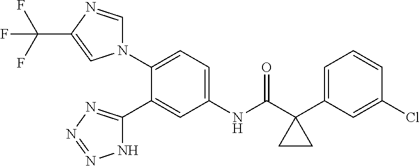 [SMILES: O=C(Nc1ccc(-n2cnc(C(F)(F)F)c2)c(-c2nnn[nH]2)c1)C1(c2cccc(Cl)c2)CC1] | 1-(3- Chlorophenyl)- N-{3-(1H- tetrazol-5-yl)-4- [4- (trifluoromethyl)- 1H-imidazol-1- yl]phenyl}cyclopro- panecarboxa- mide | ¹H NMR (500 MHz, DMSO-d6) δ [ppm] 9.71 (s, 1H), 8.27 (s, 1H), 7.93 (dd, J = 8.7, 2.4 Hz, 1H), 7.90 (s, 1H), 7.87 (s, 1H), 7.60 (d, J = 8.7 Hz, 1H), 7.47-7.44 (m, 1H), 7.43-7.35 (m, 3H), 1.57-1.46 (m, 2H), 1.28-1.17 (m, 2H). LCMS (Analytical Method F): Rt = 3.44 mins; MS MS (ESIpos) m/z = 474.1 (M + H)⁺. |
| 353 | 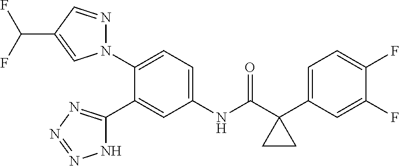 [SMILES: O=C(Nc1ccc(-n2cc(C(F)F)cn2)c(-c2nnn[nH]2)c1)C1(c2ccc(F)c(F)c2)CC1] | N-{4-[4- (Difluoromethyl)- 1H-pyrazol-1- yl]-3-(1H- tetrazol-5- yl)phenyl}-1- (3,4- difluorophenyl)cy- clopropanecar- boxamide | ¹H NMR (500 MHz, DMSO-d6) δ [ppm] 9.38 (s, 1H), 8.33 (s, 1H), 8.04 (s, 1H), 7.95 (dd, J = 8.8, 2.4 Hz, 1H), 7.75 (s, 1H), 7.63 (d, J = 8.8 Hz, 1H), 7.51 (ddd, J = 11.8, 7.8, 2.2 Hz, 1H), 7.46- 7.38 (m, 1H), 7.31-7.25 (m, 1H), 7.04 (t, J = 55.9 Hz, 1H), 1.58-1.45 (m, 2H), 1.26-1.13 (m, 2H). LCMS (Analytical Method F): Rt = |
| | | | 3.24 mins; MS (ESIpos) m/z = |
| | | | 458.1 (M + H)⁺. |
| 354 | 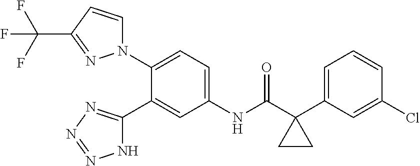 [SMILES: O=C(Nc1ccc(-n2ccc(C(F)(F)F)n2)c(-c2nnn[nH]2)c1)C1(c2cccc(Cl)c2)CC1] | 1-(3- Chlorophenyl)- N-{3-(1H- tetrazol-5-yl)-4- [3- (trifluoromethyl)- 1H-pyrazol-1- yl]phenyl}cyclopro- panecarboxa- mide | ¹H NMR (500 MHz, DMSO-d6) δ [ppm] 9.65 (s, 1H), 8.14 (s, 2H), 7.97 (dd, J = 8.8, 2.4 Hz, 1H), 7.68 (d, J = 8.8 Hz, 1H), 7.46 (d, J = 1.6 Hz, 1H), 7.43-7.34 (m, 3H), 6.88 (d, J = 2.5 Hz, 1H), 1.59-1.46 (m, 2H), 1.28-1.15 (m, 2H). LCMS (Analytical Method F): Rt = 3.62 mins; MS (ESIpos) m/z = 474.1 (M + H)⁺. |
| 355 | 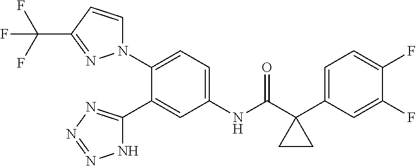 [SMILES: O=C(Nc1ccc(-n2ccc(C(F)(F)F)n2)c(-c2nnn[nH]2)c1)C1(c2ccc(F)c(F)c2)CC1] | 1-(3,4- Difluorophenyl)- N-{3-(1H- tetrazol-5-yl)-4- [3- (trifluoromethyl)- 1H-pyrazol-1- yl]phenyl}cyclopro- panecarboxa- mide | ¹H NMR (500 MHz, DMSO-d6) δ [ppm] 9.42 (s, 1H), 8.18-8.13 (m, 1H), 8.11 (s, 1H), 7.97 (dd, J = 8.8, 2.4 Hz, 1H), 7.68 (d, J = 8.8 Hz, 1H), 7.51 (ddd, J = 11.8, 7.8, 2.2 Hz, 1H), 7.47-7.38 (m, 1H), 7.29 (dq, J = 6.3, 2.2 Hz, 1H), 6.88 (d, J = 2.4 Hz, 1H), 1.59-1.45 (m, 2H), 1.27-1.14 (m, 2H). LCMS (Analytical Method F): Rt = |
| | | | 3.50 mins; MS (ESIpos) m/z = |
| | | | 476.1 (M + H)⁺. |
| 356 | 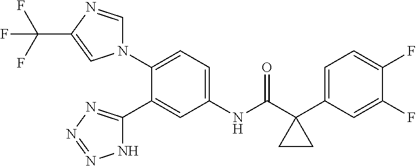 [SMILES: O=C(Nc1ccc(-n2cnc(C(F)(F)F)c2)c(-c2nnn[nH]2)c1)C1(c2ccc(F)c(F)c2)CC1] | 1-(3,4- Difluorophenyl)- N-{3-(1H- tetrazol-5-yl)-4- [4- (trifluoromethyl)- 1H-imidazol-1- yl]phenyl}cyclopro- panecarboxa- mide | ¹H NMR (500 MHz, DMSO-d6) δ [ppm] 9.42 (s, 1H), 8.18-8.13 (m, 1H), 8.11 (s, 1H), 7.97 (dd, J = 8.8, 2.4 Hz, 1H), 7.68 (d, J = 8.8 Hz, 1H), 7.51 (ddd, J = 11.8, 7.8, 2.2 Hz, 1H), 7.47-7.38 (m, 1H), 7.29 (dq, J = 6.3, 2.2 Hz, 1H), 6.88 (d, J = 2.4 Hz, 1H), 1.59-1.45 (m, 2H), 1.27-1.14 (m, 2H). LCMS (Analytical Method F): Rt = |
| | | | 3.50 mins; MS (ESIpos) m/z = |
| | | | 476.1 (M + H)⁺. |
| 357 | 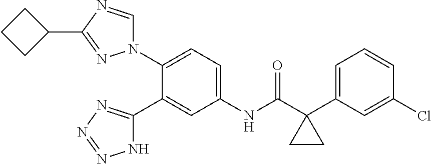 [SMILES: O=C(Nc1ccc(-n2cnc(C3CCC3)n2)c(-c2nnn[nH]2)c1)C1(c2cccc(Cl)c2)CC1] | 1-(3- chlorophenyl)- N-[4-(3- cyclobutyl-1H- 1,2,4-triazol-1- yl)-3-(1H- tetrazol-5- yl)phenyl]cyclopro- panecarboxa- mide | ¹H NMR (400 MHz, DMSO-d6) δ [ppm] = 1.20-1.23 (m, 2H), 1.50- 1.53 (m, 2H), 1.75-1.84 (m, 1H), 1.87-1.98 (m, 1H), 2.06- 2.23 (m, 4H), 3.39-3.48 (m, 1H), 7.35-7.40 (m, 3H), 7.45- 7.46 (m, 1H), 7.65 (d, 1H), 7.95 (dd, 1H), 8.12 (d, 1H), 8.62 (s, 1H), 9.64 (s, 1H). LCMS (method 1): Rt = 1.13 min; MS (ESIpos) m/z = 461. (M + H)⁺. |
| 358 | 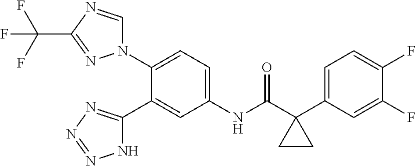 [SMILES: O=C(Nc1ccc(-n2cnc(C(F)(F)F)n2)c(-c2nnn[nH]2)c1)C1(c2ccc(F)c(F)c2)CC1] | 1-(3,4- difluorophenyl)- N-{3-(1H- tetrazol-5-yl)-4- [3- (trifluoromethyl)- 1H-1,2,4- triazol-1- yl]phenyl}cyclopro- panecarboxa- mide | ¹H NMR (400 MHz, DMSO-d6) δ [ppm] = 1.19-1.22 (m, 2H), 1.52- 1.55 (m, 2H), 7.27-7.31 (m, 1H), 7.39-7.46 (m, 1H), 7.50- 7.55 (m, 1H), 7.71 (d, 1H), 7.96 (dd, 1H), 8.31 (d, 1H), 9.07 (s, 1H), 9.50 (s, 1H). LCMS (method 1): Rt = 1.14 min; MS (ESIpos) m/z = 477 (M + H)⁺. |
| 359 | 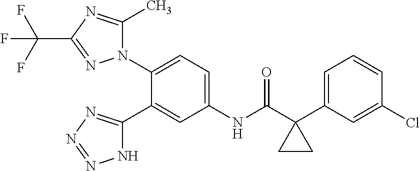 [SMILES: Cc1nc(C(F)(F)F)nn1-c1ccc(NC(=O)C2(c3cccc(Cl)c3)CC2)cc1-c1nnn[nH]1] | 1-(3- chlorophenyl)- N-{4-[5-methyl- 3- (trifluoromethyl)- 1H-1,2,4- triazol-1-yl]-3- (1H-tetrazol-5- yl)phenyl}cyclopro- panecarboxa- mide | ¹H NMR (400 MHz, DMSO-d6) δ [ppm] = 1.21-1.24 (m, 2H), 1.52- 1.55 (m, 2H), 2.23 (s, 3H), 7.36- 7.41 (m, 3H), 7.46-7.47 (m, 1H), 7.70 (d, 1H), 7.97 (dd, 1H), 8.43 (d, 1H), 9.79 (s, 1H). LCMS (method 1): Rt = 1.20 min; MS (ESIpos) m/z = 489 (M + H)⁺. |
| 360 | 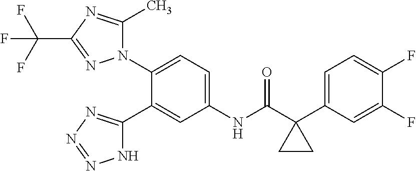 [SMILES: Cc1nc(C(F)(F)F)nn1-c1ccc(NC(=O)C2(c3ccc(F)c(F)c3)CC2)cc1-c1nnn[nH]1] | 1-(3,4- difluorophenyl)- N-{4-[5-methyl- 3- (trifluoromethyl)- 1H-1,2,4- triazol-1-yl]-3- (1H-tetrazol-5- yl)phenyl}cyclopro- panecarboxa- mide | ¹H NMR (400 MHz, DMSO-d6) δ [ppm] = 1.19-1.22 (m, 2H), 1.52- 1.54 (m, 2H), 2.19 (s, 3H), 7.29- 7.32 (m, 1H), 7.40-7.46 (m, 1H), 7.50-7.55 (m, 1H), 7.64 (d, 1H), 7.94 (dd, 1H), 8.39 (d, 1H), 9.53 (s, 1H). LCMS (method 1): Rt = 1.16 min; MS (ESIpos) m/z = 491 (M + H)⁺. |
| 361 | 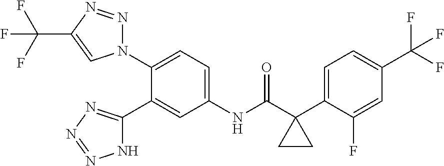 [SMILES: O=C(Nc1ccc(-n2cc(C(F)(F)F)nn2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[4- (trifluoromethyl)- 1H-1,2,3- triazol-1- yl]phenyl}cyclopro- panecarboxa- mide | ¹H NMR (400 MHz, DMSO-d6) δ [ppm] = 1.27-1.30 (m, 2H), 1.67- 1.70 (m, 2H), 7.61-7.64 (m, 2H), 7.69-7.75 (m, 2H), 7.92 (dd, 1H), 8.29 (d, 1H), 9.16 (s, 1H), 9.47 (s, 1H). LCMS (method 1): Rt = 1.25 min; MS (ESIpos) m/z = 527 (M + H)⁺. |
| 362 | 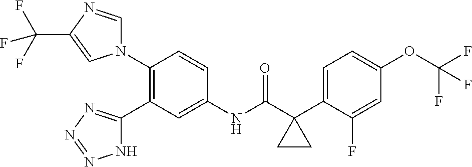 [SMILES: O=C(Nc1ccc(-n2cnc(C(F)(F)F)c2)c(-c2nnn[nH]2)c1)C1(c2ccc(OC(F)(F)F)cc2F)CC1] | 1-[2-Fluoro-4- (trifluoromethoxy) phenyl]-N-{3- (1H-tetrazol-5- yl)-4-[4- (trifluoromethyl)- 1H-imidazol-1- yl]phenyl}cyclopro- panecarboxa- mide | ¹H NMR (500 MHz, DMSO-d6) δ [ppm] 9.39 (s, 1H), 8.22 (s, 1H), 7.93-7.88 (m, 2H), 7.86 (s, 1H), 7.66-7.55 (m, 2H), 7.42-7.37 (m, 1H), 7.29-7.22 (m, 1H), 1.71-1.58 (m, 2H), 1.31-1.19 (m, 2H). LCMS (Analytical Method F): Rt = 3.66 mins; MS (ESIpos) m/z = 542.2 (M + H)⁺. |
| 363 | 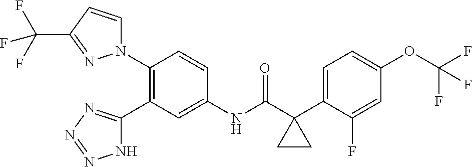 [SMILES: O=C(Nc1ccc(-n2ccc(C(F)(F)F)n2)c(-c2nnn[nH]2)c1)C1(c2ccc(OC(F)(F)F)cc2F)CC1] | 1-[2-Fluoro-4- (trifluoromethoxy) phenyl]-N-{3- (1H-tetrazol-5- yl)-4-[3- (trifluoromethyl)- 1H-pyrazol-1- yl]phenyl}cyclopro- panecarboxa- mide | ¹H NMR (500 MHz, DMSO-d6) δ [ppm] 9.37 (s, 1H), 8.19-8.13 (m, 1H), 8.10 (s, 1H), 7.96 (dd, J = 8.8, 2.4 Hz, 1H), 7.68 (d, J = 8.8 Hz, 1H), 7.65-7.58 (m, 1H), 7.39 (dd, J = 10.4, 1.8 Hz, 1H), 7.26 (d, J = 8.6 Hz, 1H), 6.88 (d, J = 2.4 Hz, 1H), 1.70-1.59 (m, 2H), 1.29-1.18 (m, 2H). LCMS (Analytical Method F): Rt = 3.83 mins; MS (ESIpos) m/z = |
| | | | 542.2 (M + H)⁺. |
| 364 | 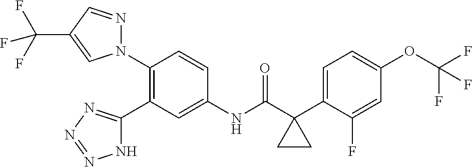 [SMILES: O=C(Nc1ccc(-n2cc(C(F)(F)F)cn2)c(-c2nnn[nH]2)c1)C1(c2ccc(OC(F)(F)F)cc2F)CC1] | 1-[2-fluoro-4- (trifluoromethoxy) phenyl]-N-{3- (1H-tetrazol-5- yl)-4-[4- (trifluoromethyl)- 1H-pyrazol-1- yl]phenyl}cyclopro- panecarboxa- mide | ¹H NMR (500 MHz, DMSO-d6) δ [ppm] 9.36 (s, 1H), 8.72 (s, 1H), 8.08 (s, 1H), 7.96 (s, 1H), 7.94 (dd, J = 8.8, 2.4 Hz, 1H), 7.66 (d, J = 8.8 Hz, 1H), 7.64-7.59 (m, 1H), 7.43-7.36 (m, 1H), 7.29- 7.23 (m, 1H), 1.70-1.59 (m, 2H), 1.29-1.18 (m, 2H). LCMS (Analytical Method F): Rt = 3.83 mins; MS (ESIpos) m/z = 542.2 (M + H)⁺. |
| 365 | 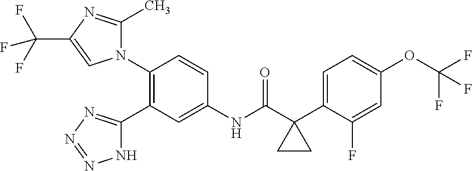 [SMILES: Cc1nc(C(F)(F)F)cn1-c1ccc(NC(=O)C2(c3ccc(OC(F)(F)F)cc3F)CC2)cc1-c1nnn[nH]1] | 1-[2-Fluoro-4- (trifluoromethoxy) phenyl]-N-{4- [2-methyl-4- (trifluoromethyl)- 1H-imidazol-1- yl]-3-(1H- tetrazol-5- yl)phenyl}cyclopro- panecarboxa- mide | ¹H NMR (500 MHz, DMSO-d6) δ [ppm] 9.40 (s, 1H), 8.31 (s, 1H), 7.91 (dd, J = 8.7, 2.4 Hz, 1H), 7.71-7.67 (m, 1H), 7.66-7.59 (m, 1H), 7.56 (d, J = 8.7 Hz, 1H), 7.43-7.37 (m, 1H), 7.29-7.24 (m, 1H), 1.99 (s, 3H), 1.73-1.58 (m, 2H), 1.33-1.17 (m, 2H). LCMS (Analytical Method F): Rt = 3.37 mins; MS (ESIpos) m/z = 556.2 (M + H)⁺. |
| 366 | 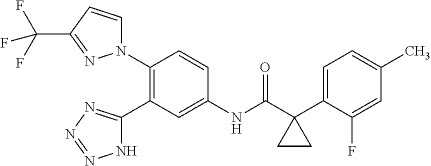 [SMILES: Cc1ccc(C2(C(=O)Nc3ccc(-n4ccc(C(F)(F)F)n4)c(-c4nnn[nH]4)c3)CC2)c(F)c1] | 1-(2-Fluoro-4- methylphenyl)- N-{3-(1H- tetrazol-5-yl)-4- [3- (trifluoromethyl)- 1H-pyrazol-1- yl]phenyl}cyclopro- panecarboxa- mide | ¹H NMR (500 MHz, DMSO-d6) δ [ppm] 9.25 (s, 1H), 8.15 (s, 1H), 8.10 (s, 1H), 7.97 (dd, J = 8.8, 2.4 Hz, 1H), 7.66 (d, J = 8.8 Hz, 1H), 7.38-7.31 (m, 1H), 7.05 (d, J = 3.9 Hz, 1H), 7.03 (s, 1H), 6.87 (d, J = 2.5 Hz, 1H), 2.34 (s, 3H), 1.65-1.53 (m, 2H), 1.21- 1.09 (m, 2H). LCMS (Analytical Method F): Rt = 3.58 mins; MS (ESIpos) m/z = |
| | | | 472.2 (M + H)⁺. |
| 367 | 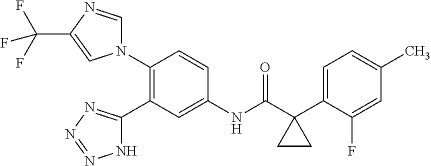 [SMILES: Cc1ccc(C2(C(=O)Nc3ccc(-n4cnc(C(F)(F)F)c4)c(-c4nnn[nH]4)c3)CC2)c(F)c1] | 1-(2-Fluoro-4- methylphenyl)- N-{3-(1H- tetrazol-5-yl)-4- [4- (trifluoromethyl)- 1H-imidazol-1- yl]phenyl}cyclopro- panecarboxa- mide | ¹H NMR (500 MHz, DMSO-d6) δ [ppm] 9.26 (s, 1H), 8.22 (s, 1H), 7.93 (dd, J = 8.8, 2.4 Hz, 1H), 7.89 (s, 1H), 7.86 (s, 1H), 7.57 (d, J = 8.7 Hz, 1H), 7.40-7.29 (m, 1H), 7.07-7.04 (m, 1H), 7.03 (s, 1H), 2.34 (s, 3H), 1.66- 1.53 (m, 2H), 1.21-1.10 (m, 2H). LCMS (Analytical Method F): Rt = 3.40 mins; MS (ESIpos) m/z = |
| | | | 472.2 (M + H)⁺. |
| 368 | 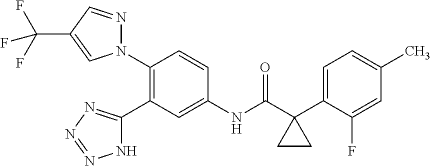 [SMILES: Cc1ccc(C2(C(=O)Nc3ccc(-n4cc(C(F)(F)F)cn4)c(-c4nnn[nH]4)c3)CC2)c(F)c1] | 1-(2-Fluoro-4- methylphenyl)- N-{3-(1H- tetrazol-5-yl)-4- [4- (trifluoromethyl)- 1H-pyrazol-1- yl]phenyl}cyclopro- panecarboxa- mide | ¹H NMR (500 MHz, DMSO-d6) δ [ppm] 9.23 (s, 1H), 8.71 (s, 1H), 8.09 (d, J = 1.8 Hz, 1H), 7.98- 7.93 (m, 2H), 7.64 (d, J = 8.8 Hz, 1H), 7.40-7.28 (m, 1H), 7.07- 7.04 (m, 1H), 7.03 (s, 1H), 2.34 (s, 3H), 1.64-1.53 (m, 2H), 1.20- 1.09 (m, 2H). LCMS (Analytical Method F): Rt = 3.58 mins; MS (ESIpos) m/z = 472.2 (M +H)⁺. |
| 369 | 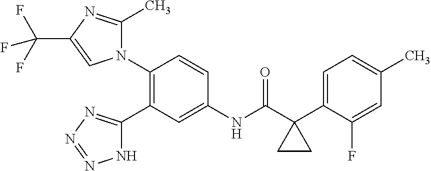 [SMILES: Cc1ccc(C2(C(=O)Nc3ccc(-n4cc(C(F)(F)F)nc4C)c(-c4nnn[nH]4)c3)CC2)c(F)c1] | 1-(2-Fluoro-4- methylphenyl)- N-{4-[2-methyl- 4- (trifluoromethyl)- 1H-imidazol-1- yl]-3-(1H- tetrazol-5- yl)phenyl}cyclopro- panecarboxa- mide | ¹H NMR (500 MHz, DMSO-d6) δ [ppm] 9.28 (s, 1H), 8.31 (s, 1H), 7.93 (dd, J = 8.7, 2.4 Hz, 1H), 7.75-7.64 (m, 1H), 7.55 (d, J = 8.7 Hz, 1H), 7.43-7.29 (m, 1H), 7.05 (s, 1H), 7.04 (s, 1H), 2.34 (s, 3H), 1.99 (s, 3H), 1.70-1.51 (m, 2H), 1.24-1.08 (m, 2H). LCMS (Analytical Method F): Rt = 3.46 mins; MS (ESIpos) m/z = 486.2 (M + H)⁺. |
| 370 | 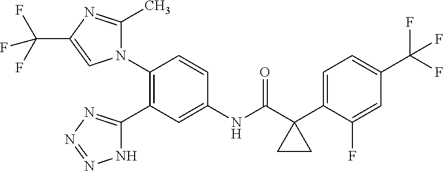 [SMILES: Cc1nc(C(F)(F)F)cn1-c1ccc(NC(=O)C2(c3ccc(C(F)(F)F)cc3F)CC2)cc1-c1nnn[nH]1] | 1-[2-Fluoro-4- (trifluoromethyl) phenyl]-N-{4- [2-methyl-4- (trifluoromethyl)- 1H-imidazol-1- yl]-3-(1H- tetrazol-5- yl)phenyl}cyclopro- panecarboxa- mide | ¹H NMR (500 MHz, DMSO-d6) δ [ppm] 9.44 (s, 1H), 8.31 (s, 1H), 7.92 (dd, J = 8.7, 2.4 Hz, 1H), 7.77-7.66 (m, 3H), 7.66-7.60 (m, 1H), 7.57 (d, J = 8.7 Hz, 1H), 2.00 (s, 3H), 1.75-1.61 (m, 2H), 1.36-1.21 (m, 2H). LCMS (Analytical Method F): Rt = 3.64 mins; MS (ESIpos) m/z = 540.1 (M + H)⁺. |
| 371 | 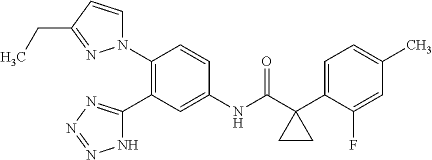 [SMILES: CCc1ccn(-c2ccc(NC(=O)C3(c4ccc(C)cc4F)CC3)cc2-c2nnn[nH]2)n1] | N-[4-(3-ethyl- 1H-pyrazol-1- yl)-3-(1H- tetrazol-5- yl)phenyl]-1-(2- fluoro-4- methylphenyl)cyclo- propanecar- boxamide | ¹H NMR (500 MHz, DMSO-d6) δ [ppm] 9.11 (s, 1H), 7.92 (dd, J = 8.8, 2.4 Hz, 1H), 7.87 (d, J = 2.3 Hz, 1H), 7.72 (d, J = 2.3 Hz, 1H), 7.58 (d, J = 8.8 Hz, 1H), 7.38- 7.29 (m, 1H), 7.08-6.97 (m, 2H), 6.20 (d, J = 2.4 Hz, 1H), 2.42 (q, J = 7.6 Hz, 2H), 2.33 (s, 3H), 1.63-1.51 (m, 2H), 1.18- 1.07 (m, 2H), 1.02 (t, J = 7.6 Hz, |
| | | | 3H). |
| | | | LCMS (Analytical Method F): Rt = |
| | | | 3.37 mins; MS (ESIpos) m/z = |
| | | | 432.1 (M + H)⁺. |
| 372 | 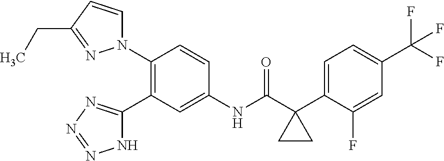 [SMILES: CCc1ccn(-c2ccc(NC(=O)C3(c4ccc(C(F)(F)F)cc4F)CC3)cc2-c2nnn[nH]2)n1] | N-[4-(3-Ethyl- 1H-pyrazol-1- yl)-3-(1H- tetrazol-5- yl)phenyl]-1-[2- fluoro-4- (trifluoromethyl) phenyl]cyclopro- panecarboxamide | ¹H NMR (500 MHz, DMSO-d6) δ [ppm] 9.27 (s, 1H), 7.90 (dd, J = 8.8, 2.4 Hz, 1H), 7.86 (s, 1H), 7.78-7.65 (m, 3H), 7.63-7.56 (m, 2H), 6.21 (d, J = 2.3 Hz, 1H), 2.42 (q, J = 7.6 Hz, 2H), 1.72- 1.59 (m, 2H), 1.32-1.18 (m, 2H), 1.02 (t, J = 7.6 Hz, 3H). LCMS (Analytical Method F): Rt = 3.57 mins; MS (ESIpos) m/z = |
| | | | 486.2 (M + H)⁺. |
| 373 | 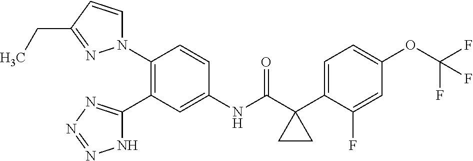 [SMILES: CCc1ccn(-c2ccc(NC(=O)C3(c4ccc(OC(F)(F)F)cc4F)CC3)cc2-c2nnn[nH]2)n1] | N-[4-(3-Ethyl- 1H-pyrazol-1- yl)-3-(1H- tetrazol-5- yl)phenyl]-1-[2- fluoro-4- (trifluoromethoxy) phenyl]cyclo- propanecarboxa- mide | ¹H NMR (500 MHz, DMSO-d6) δ [ppm] 9.24 (s, 1H), 7.90 (dd, J = 8.8, 2.4 Hz, 1H), 7.88-7.84 (m, 1H), 7.73 (d, J = 2.1 Hz, 1H), 7.64-7.57 (m, 2H), 7.41-7.35 (m, 1H), 7.25 (d, J = 8.5 Hz, 1H), 6.21 (d, J = 2.4 Hz, 1H), 2.42 (q, J = 7.6 Hz, 2H), 1.68-1.57 (m, 2H), 1.28-1.16 (m, 2H), 1.02 (t, J = 7.6 Hz, 3H). |
| | | | LCMS (Analytical Method F): Rt = |
| | | | 3.66 mins; MS (ESIpos) m/z = |
| | | | 502.2 (M + H)⁺. |
| 374 | 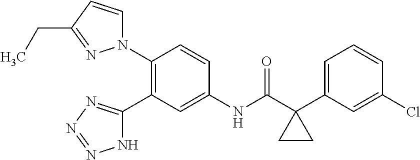 [SMILES: CCc1ccn(-c2ccc(NC(=O)C3(c4cccc(Cl)c4)CC3)cc2-c2nnn[nH]2)n1] | 1-(3- Chlorophenyl)- N-[4-(3-ethyl- 1H-pyrazol-1- yl)-3-(1H- tetrazol-5- yl)phenyl]cyclopro- panecarboxa- mide | ¹H NMR (500 MHz, DMSO-d6) δ [ppm] 9.52 (s, 1H), 7.95-7.88 (m, 2H), 7.73 (d, J = 2.0 Hz, 1H), 7.63-7.57 (m, 1H), 7.47-7.43 (m, 1H), 7.42-7.33 (m, 3H), 6.21 (d, J = 2.4 Hz, 1H), 2.42 (q, J = 7.6 Hz, 2H), 1.56-1.44 (m, 2H), 1.27-1.13 (m, 2H), 1.02 (t, J = 7.6 Hz, 3H). LCMS (Analytical Method F): RT = |
| | | | 3.42 mins; MS (ESIpos) m/z = |
| | | | 434.1 (M + H)⁺. |
| 375 | 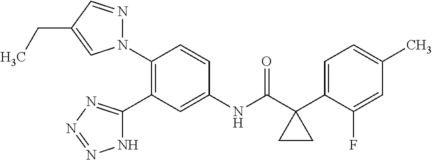 [SMILES: CCc1cnn(-c2ccc(NC(=O)C3(c4ccc(C)cc4F)CC3)cc2-c2nnn[nH]2)c1] | N-[4-(4-Ethyl- 1H-pyrazol-1- yl)-3-(1H- tetrazol-5- yl)phenyl]-1-(2- fluoro-4- methylphenyl)cy- clopropanecar- boxamide | ¹H NMR (500 MHz, DMSO-d6) δ [ppm] 9.12 (s, 1H), 7.93 (dd, J = 8.8, 2.4 Hz, 1H), 7.89 (d, J = 2.1 Hz, 1H), 7.70 (s, 1H), 7.57 (d, J = 8.8 Hz, 1H), 7.38-7.31 (m, 2H), 7.07-7.04 (m, 1H), 7.03 (s, 1H), 2.43 (q, J = 7.5 Hz, 2H), 2.34 (s, 3H), 1.57 (q, J = 4.1 Hz, 2H), 1.17-1.10 (m, 5H). LCMS (Analytical Method F): Rt = |
| | | | 3.46 mins; MS (ESIpos) m/z = |
| | | | 432.2 (M + H)⁺. |
| 376 | 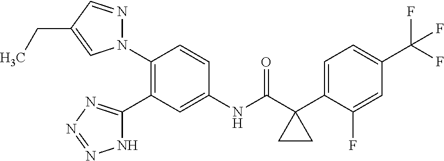 [SMILES: CCc1cnn(-c2ccc(NC(=O)C3(c4ccc(C(F)(F)F)cc4F)CC3)cc2-c2nnn[nH]2)c1] | N-[4-(4-Ethyl- 1H-pyrazol-1- yl)-3-(1H- tetrazol-5- yl)phenyl]-1-[2- fluoro-4- (trifluoromethyl) phenyl]cyclopro- panecarboxamide | ¹H NMR (500 MHz, DMSO-d6) δ [ppm] 9.28 (s, 1H), 7.90 (dd, J = 8.8, 2.4 Hz, 1H), 7.87 (d, J = 2.2 Hz, 1H), 7.75-7.65 (m, 3H), 7.63-7.55 (m, 2H), 7.36 (s, 1H), 2.43 (q, J = 7.5 Hz, 2H), 1.72- 1.59 (m, 2H), 1.31-1.20 (m, 2H), 1.13 (t, J = 7.6 Hz, 3H). LCMS (Analytical Method F): Rt = 3.65 mins; MS (ESIpos) m/z = |
| | | | 486.1 (M + H)⁺. |
| 377 | 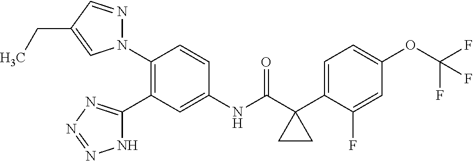 [SMILES: CCc1cnn(-c2ccc(NC(=O)C3(c4ccc(OC(F)(F)F)cc4F)CC3)cc2-c2nnn[nH]2)c1] | N-[4-(4-ethyl- 1H-pyrazol-1- yl)-3-(1H- tetrazol-5- yl)phenyl]-1-[2- fluoro-4- (trifluoromethoxy) phenyl]cyclo- propanecarboxa- mide | ¹H NMR (500 MHz, DMSO-d6) δ [ppm] 9.25 (s, 1H), 7.90 (dd, J = 8.8, 2.4 Hz, 1H), 7.89-7.85 (m, 1H), 7.70 (s, 1H), 7.64-7.54 (m, 2H), 7.41-7.37 (m, 1H), 7.36 (s, 1H), 7.28-7.22 (m, 1H), 2.43 (q, J = 7.5 Hz, 2H), 1.68-1.56 (m, 2H), 1.27-1.17 (m, 2H), 1.13 (t, J = 7.6 Hz, 3H). LCMS (Analytical Method F): Rt = |
| | | | 3.73 mins; MS (ESIpos) m/z = |
| | | | 502.1 (M + H)⁺. |
| 378 | 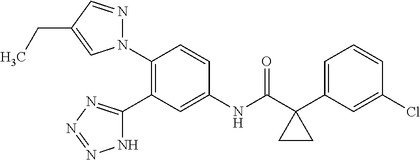 [SMILES: CCc1cnn(-c2ccc(NC(=O)C3(c4cccc(Cl)c4)CC3)cc2-c2nnn[nH]2)c1] | 1-(3- Chlorophenyl)- N-[4-(4-ethyl- 1H-pyrazol-1- yl)-3-(1H- tetrazol-5- yl)phenyl]cyclopro- panecarboxa- mide | ¹H NMR (500 MHz, DMSO-d6) δ [ppm] 9.53 (s, 1H), 7.96-7.89 (m, 2H), 7.70 (s, 1H), 7.59 (d, J = 8.9 Hz, 1H), 7.47-7.43 (m, 1H), 7.42-7.33 (m, 4H), 2.43 (q, J = 7.5 Hz, 2H), 1.55-1.45 (m, 2H), 1.25-1.17 (m, 2H), 1.13 (t, J = 7.6 Hz, 3H). LCMS (Analytical Method F): Rt = 3.50 mins; MS (ESIpos) m/z = |
| | | | 434.1 (M + H)⁺. |
| 379 | 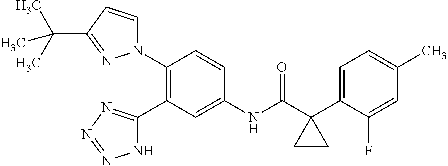 [SMILES: Cc1ccc(C2(C(=O)Nc3ccc(-n4ccc(C(C)(C)C)n4)c(-c4nnn[nH]4)c3)CC2)c(F)c1] | N-[4-(3-tert- Butyl-1H- pyrazol-1-yl)-3- (1H-tetrazol-5- yl)phenyl]-1-(2- fluoro-4- methylphenyl)cy- clopropanecar- boxamide | ¹H NMR (500 MHz, DMSO-d6) δ [ppm] 9.10 (s, 1H), 7.93 (dd, J = 8.9, 2.4 Hz, 1H), 7.89 (s, 1H), 7.85-7.79 (m, 1H), 7.61 (d, J = 8.8 Hz, 1H), 7.37-7.30 (m, 1H), 7.06-7.01 (m, 2H), 6.28 (d, J = 2.4 Hz, 1H), 2.33 (s, 3H), 1.63- 1.50 (m, 2H), 1.17-1.07 (m, 2H), 1.03 (s, 9H). LCMS (Analytical Method F): Rt = 3.76 mins; MS (ESIpos) m/z = |
| | | | 460.2 (M + H)⁺. |
| 380 | 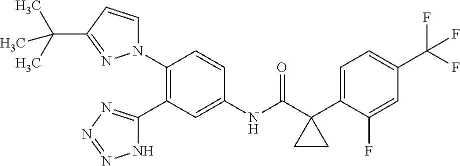 [SMILES: CC(C)(C)c1ccn(-c2ccc(NC(=O)C3(c4ccc(C(F)(F)F)cc4F)CC3)cc2-c2nnn[nH]2)n1] | N-[4-(3-tert- Butyl-1H- pyrazol-1-yl)-3- (1H-tetrazol-5- yl)phenyl]-1-[2- fluoro-4- (trifluoromethyl) phenyl]cyclopro- panecarboxamide | ¹H NMR (500 MHz, DMSO-d6) δ [ppm] 9.26 (s, 1H), 7.95-7.85 (m, 2H), 7.81 (s, 1H), 7.74-7.66 (m, 2H), 7.65-7.57 (m, 2H), 6.28 (d, J = 2.3 Hz, 1H), 1.72- 1.58 (m, 2H), 1.31-1.18 (m, 2H), 1.04 (s, 9H). LCMS (Analytical Method F): Rt = 3.93 mins; MS (ESIpos) m/z = 514.3 (M + H)⁺. |
| 381 | 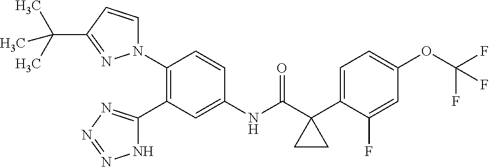 [SMILES: CC(C)(C)c1ccn(-c2ccc(NC(=O)C3(c4ccc(OC(F)(F)F)cc4F)CC3)cc2-c2nnn[nH]2)n1] | N-[4-(3-tert- Butyl-1H- pyrazol-1-yl)-3- (1H-tetrazol-5- yl)phenyl]-1-[2- fluoro-4- (trifluoromethoxy) phenyl]cyclo- propanecarboxa- mide | ¹H NMR (500 MHz, DMSO-d6) δ [ppm] 9.23 (s, 1H), 7.94-7.86 (m, 2H), 7.81 (s, 1H), 7.66-7.56 (m, 2H), 7.38 (dd, J = 10.3, 1.8 Hz, 1H), 7.28-7.21 (m, 1H), 6.28 (d, J = 2.4 Hz, 1H), 1.68- 1.56 (m, 2H), 1.27-1.16 (m, 2H), 1.04 (s, 9H). LCMS (Analytical Method F): Rt = 4.00 mins; MS (ESIpos) m/z = 530.3 (M + H)⁺. |
| 382 | 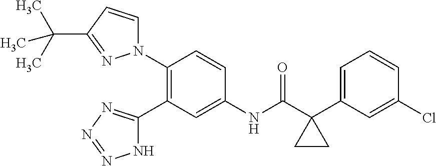 [SMILES: CC(C)(C)c1ccn(-c2ccc(NC(=O)C3(c4cccc(Cl)c4)CC3)cc2-c2nnn[nH]2)n1] | N-[4-(3-tert- Butyl-1H- pyrazol-1-yl)-3- (1H-tetrazol-5- yl)phenyl]-1-(3- chlorophenyl)cyclo- propanecarbox- amide | ¹H NMR (500 MHz, DMSO-d6) δ [ppm] 9.50 (s, 1H), 7.95-7.83 (m, 3H), 7.63 (d, J = 8.8 Hz, 1H), 7.44 (d, J = 1.5 Hz, 1H), 7.42- 7.33 (m, 3H), 6.28 (d, J = 2.4 Hz, 1H), 1.57-1.43 (m, 2H), 1.25- 1.13 (m, 2H), 1.03 (s, 9H). LCMS (Analytical Method F): Rt = 3.81 mins; MS (ESIpos) m/z = 462.2 (M + H)⁺. |
| 383 | 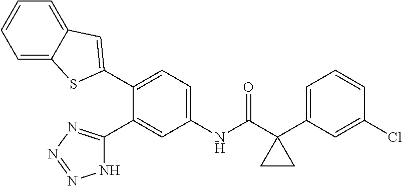 [SMILES: O=C(Nc1ccc(-c2cc3ccccc3s2)c(-c2nnn[nH]2)c1)C1(c2cccc(Cl)c2)CC1] | N-[4-(1- benzothiophen- 2-yl)-3-(1H- tetrazol-5- yl)phenyl]-1-(3- chlorophenyl)cyclo- propanecarbox- amide | ¹H NMR (500 MHz, DMSO-d6) δ [ppm] 1.21 (q, J = 4.5 Hz, 2H), 1.52 (q, J = 4.4 Hz, 2H), 7.14 (s, 1H), 7.28-7.42 (m, 5H), 7.46 (d, J = 1.6 Hz, 1H), 7.75 (dd, J = 17.0, 7.8 Hz, 2H), 7.85-7.99 (m, 3H), 9.58 (s, 1H). LCMS (Analytical Method D) Rt = 4.78 mins; MS (ESIpos) m/z = 472.0 (M + H)⁺. |
| 384 | 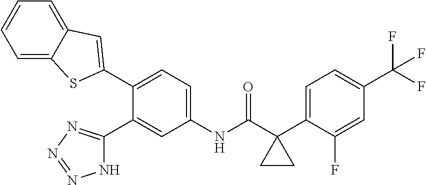 [SMILES: O=C(Nc1ccc(-c2cc3ccccc3s2)c(-c2nnn[nH]2)c1)C1(c2ccc(C(F)(F)F)cc2F)CC1] | N-[4-(1- benzothiophen- 2-yl)-3-(1H- tetrazol-5- yl)phenyl]-1-[2- fluoro-4- (trifluoromethyl) phenyl]cyclopro- panecarboxamide | ¹H NMR (500 MHz, DMSO-d6) δ [ppm] 1.19-1.34 (m, 2H), 1.67 (q, J = 4.3 Hz, 2H), 7.14 (s, 1H), 7.34 (td, J = 7.9, 1.3 Hz, 2H), 7.61 (d, J = 7.9 Hz, 1H), 7.65- 7.79 (m, 4H), 7.85 (d, J = 2.1 Hz, 1H), 7.87-7.96 (m, 2H), 9.31 (s, 1H). LCMS (Analytical Method D) RT = 4.86 mins; MS (ESIpos) m/z = 524.1 (M + H)⁺. |
| 385 | 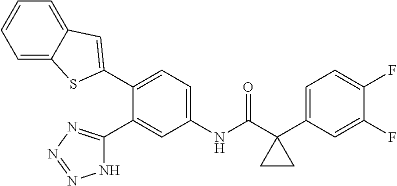 [SMILES: O=C(Nc1ccc(-c2cc3ccccc3s2)c(-c2nnn[nH]2)c1)C1(c2ccc(F)c(F)c2)CC1] | N-[4-(1- benzothiophen- 2-yl)-3-(1H- tetrazol-5- yl)phenyl]-1- (3,4- difluorophenyl)cyclo- propanecar- boxamide | ¹H NMR (500 MHz, DMSO-d6) δ 9.34 (s, 1H), 7.94 (dd, J = 8.6, 2.3 Hz, 1H), 7.91-7.86 (m, 2H), 7.78-7.75 (m, 1H), 7.73 (d, J = 8.6 Hz, 1H), 7.50 (m, 1H), 7.46- 7.38 (m, 1H), 7.37-7.30 (m, 2H), 7.30-7.25 (m, 1H), 7.13 (s, 1H), 1.57-1.45 (m, 2H), 1.25- 1.12 (m, 2H). LCMS (Analytical Method D): Rt = 3.87 min; m/z (ESIpos) = 474.1 (M + H)⁺. |
| 386 | 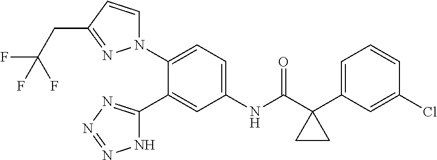 [SMILES: O=C(Nc1ccc(-n2ccc(CC(F)(F)F)n2)c(-c2nnn[nH]2)c1)C1(c2cccc(Cl)c2)CC1] | 1-(3- chlorophenyl)- N-{3-(1H- tetrazol-5-yl)-4- [3-(2,2,2- trifluoroethyl)- 1H-pyrazol-1- yl]phenyl}cyclopro- panecarboxa- mide | ¹H NMR (500 MHz, DMSO-d6) δ 9.56 (s, 1H), 7.99 (d, J = 2.1 Hz, 1H), 7.94 (dd, J = 8.8, 2.4 Hz, 1H), 7.87 (d, J = 2.3 Hz, 1H), 7.61 (d, J = 8.8 Hz, 1H), 7.47-7.43 (m, 1H), 7.42-7.34 (m, 3H), 6.38 (d, J = 2.3 Hz, 1H), 3.51 (q, J = 11.4 Hz, 2H), 1.58-1.44 (m, 2H), 1.28- 1.13 (m, 2H). LCMS (Analytical Method F): Rt = |
| | | | 3.52 mins; MS (ESIpos) m/z = |
| | | | 488.1 (M + H)⁺. |
| 387 | 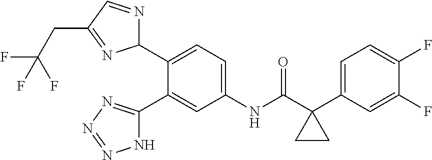 [SMILES: O=C(Nc1ccc(C2N=CC(CC(F)(F)F)=N2)c(-c2nnn[nH]2)c1)C1(c2ccc(F)c(F)c2)CC1] | 1-(3,4- difluorophenyl)- N-{3-(1H- tetrazol-5-yl)-4- [3-(2,2,2- trifluoroethyl)- 1H-pyrazol-1- yl]phenyl}cyclopro- panecarboxa- mide | ¹H NMR (500 MHz, DMSO-d6) δ 9.34 (s, 1H), 7.97 (s, 1H), 7.94 (dd, J = 8.8, 2.4 Hz, 1H), 7.88 (d, J = 2.1 Hz, 1H), 7.61 (d, J = 8.8 Hz, 1H), 7.51 (ddd, J = 11.8, 7.8, 2.1 Hz, 1H), 7.46-7.37 (m, 1H), 7.32-7.24 (m, 1H), 6.38 (d, J = 2.3 Hz, 1H), 3.50 (q, J = 11.4 Hz, 2H), 1.58-1.44 (m, 2H), 1.27- 1.12 (m, 2H). |
| | | | LCMS (Analytical Method F): Rt = |
| | | | 3.41 mins, MS (ESIpos) m/z = |
| | | | 490.2 (M + H)⁺. |
| 388 | 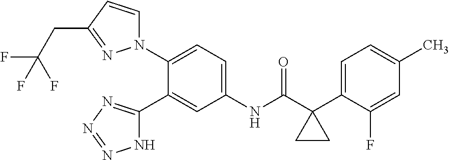 [SMILES: Cc1ccc(C2(C(=O)Nc3ccc(-n4ccc(CC(F)(F)F)n4)c(-c4nnn[nH]4)c3)CC2)c(F)c1] | 1-(2-fluoro-4- methylphenyl)- N-{3-(1H- tetrazol-5-yl)-4- [3-(2,2,2- trifluoroethyl)- 1H-pyrazol-1- yl]phenyl}cyclopro- panecarboxa- mide | ¹H NMR (500 MHz, DMSO-d6) δ 9.16 (s, 1H), 7.99-7.91 (m, 2H), 7.88 (d, J = 2.1 Hz, 1H), 7.59 (d, J = 8.8 Hz, 1H), 7.40-7.28 (m, 1H), 7.07-7.01 (m, 2H), 6.37 (d, J = 2.3 Hz, 1H), 3.50 (q, J = 11.4 Hz, 2H), 2.33 (s, 3H), 1.63-1.51 (m, 2H), 1.19-1.06 (m, 2H). LCMS (Analytical Method F): Rt = 3.48 mins, MS (ESIpos) m/z = |
| | | | 486.1 (M + H)⁺. |
| 389 | 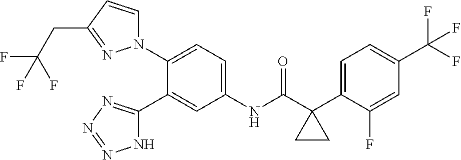 [SMILES: O=C(Nc1ccc(-n2ccc(CC(F)(F)F)n2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[3-(2,2,2- trifluoroethyl)- 1H-pyrazol-1- yl]phenyl}cyclo- propanecarboxa- mide | ¹H NMR (500 MHz, DMSO-d6) δ 9.32 (s, 1H), 7.98-7.85 (m, 3H), 7.75-7.66 (m, 2H), 7.64-7.58 (m, 2H), 6.38 (d, J = 2.1 Hz, 1H), 3.51 (q, J = 11.4 Hz, 2H), 1.74- 1.58 (m, 2H), 1.34-1.17 (m, 2H). LCMS (Analytical Method F): Rt = 3.67 mins, MS (ESIpos) m/z = 540.1 (M + H)⁺. |
| 390 | 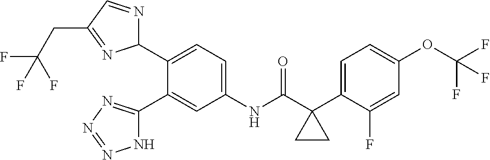 [SMILES: O=C(Nc1ccc(C2N=CC(CC(F)(F)F)=N2)c(-c2nnn[nH]2)c1)C1(c2ccc(OC(F)(F)F)cc2F)CC1] | 1-[2-fluoro-4- (trifluoromethoxy)- phenyl]-N-{3- (1H-tetrazol-5- yl)-4-[3-(2,2,2- trifluoroethyl)- 1H-pyrazol-1- yl]phenyl}cyclopro- panecarboxa- mide | ¹H NMR (500 MHz, DMSO-d6) δ 9.29 (s, 1H), 7.98-7.86 (m, 3H), 7.65-7.57 (m, 2H), 7.39 (dd, J = 10.4, 1.7 Hz, 1H), 7.25 (d, J = 8.6 Hz, 1H), 6.38 (d, J = 2.3 Hz, 1H), 3.51 (q, J = 11.4 Hz, 2H), 1.71- 1.57 (m, 2H), 1.31-1.15 (m, 2H). LCMS (Analytical Method F): Rt = 3.74 mins, MS (ESIpos) m/z = 556.1 (M + H)⁺. |
| 391 | 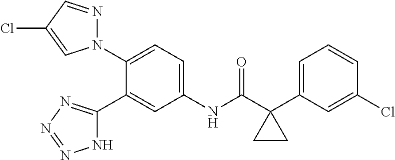 [SMILES: O=C(Nc1ccc(-n2cc(Cl)cn2)c(-c2nnn[nH]2)c1)C1(c2cccc(Cl)c2)CC1] | 1-(3- chlorophenyl)- N-[4-(4-chloro- 1H-pyrazol-1- yl)-3-(1H- tetrazol-5- yl)phenyl]cyclopro- panecarboxa- mide | ¹H NMR (500 MHz, DMSO-d6) δ 9.60 (s, 1H), 8.27 (s, 1H), 8.05 (s, 1H), 7.94 (dd, J = 8.8, 2.4 Hz, 1H), 7.67-7.57 (m, 2H), 7.49-7.32 (m, 4H), 1.56-1.46 (m, 2H), 1.27- 1.13 (m, 2H). LCMS (Analytical Method F): Rt = 3.45 mins, MS (ESIpos): m/z = 440 (M + H)⁺. |
| 393 | 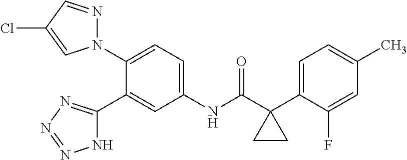 [SMILES: Cc1ccc(C2(C(=O)Nc3ccc(-n4cc(Cl)cn4)c(-c4nnn[nH]4)c3)CC2)c(F)c1] | N-[4-(4-chloro- 1H-pyrazol-1- yl)-3-(1H- tetrazol-5- yl)phenyl]-1-(2- fluoro-4- methylphenyl)cy- clopropanecar- boxamide | ¹H NMR (500 MHz, DMSO-d6) δ 9.19 (s, 1H), 8.32-8.23 (m, 1H), 8.05-7.98 (m, 1H), 7.98-7.89 (m, 1H), 7.67-7.52 (m, 2H), 7.40- 7.27 (m, 1H), 7.09-6.97 (m, 2H), 2.33 (s, 3H), 1.63-1.52 (m, 2H), 1.20-1.08 (m, 2H). LCMS (Analytical Method F): Rt = 3.41 mins, MS (ESIpos): m/z = 438 (M + H)⁺. |
| 394 | 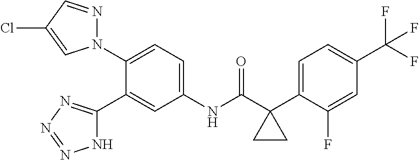 [SMILES: O=C(Nc1ccc(-n2cc(Cl)cn2)c(-c2nnn[nH]2)c1)C1(c2ccc(C(F)(F)F)cc2F)CC1] | N-[4-(4-chloro- 1H-pyrazol-1- yl)-3-(1H- tetrazol-5- yl)phenyl]-1-[2- fluoro-4- (trifluoromethyl) phenyl]cyclopro- panecarboxamide | ¹H NMR (500 MHz, DMSO-d6) δ 9.37-9.29 (m, 1H), 8.33-8.20 (m, 1H), 8.02-7.96 (m, 1H), 7.92 (dd, J = 8.8, 2.4 Hz, 1H), 7.76-7.65 (m, 2H), 7.65-7.56 (m, 3H), 1.72- 1.60 (m, 2H), 1.34-1.18 (m, 2H). LCMS (Analytical Method F): Rt = 3.60 mins, MS (ESIpos): m/z = 492 (M + H)⁺. |
| 395 | 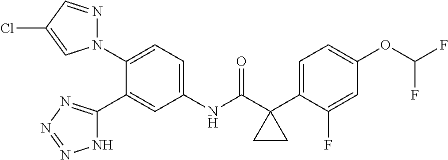 [SMILES: O=C(Nc1ccc(-n2cc(Cl)cn2)c(-c2nnn[nH]2)c1)C1(c2ccc(OC(F)F)cc2F)CC1] | N-[4-(4-chloro- 1H-pyrazol-1- yl)-3-(1H- tetrazol-5- yl)phenyl]-1-[2- fluoro-4- (trifluoromethoxy) phenyl]cyclo- propanecarboxa- mide | ¹H NMR (500 MHz, DMSO-d6) δ 9.32 (s, 1H), 8.32-8.25 (m ,1H), 8.03-7.96 (m, 1H), 7.96-7.87 (m, 1H), 7.66-7.55 (m, 3H), 7.42- 7.34 (m, 1H), 7.28-7.22 (m, 1H), 1.68-1.59 (m, 2H), 1.30- 1.15 (m, 2H). LCMS (Analytical Method F): Rt = 3.68 mins, MS (ESIpos): m/z = 508 (M + H)⁺. |
| 396 | 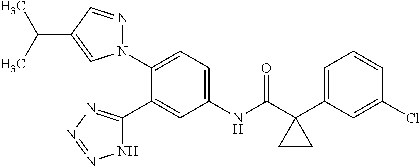 [SMILES: CC(C)c1cnn(-c2ccc(NC(=O)C3(c4cccc(Cl)c4)CC3)cc2-c2nnn[nH]2)c1] | 1-(3- chlorophenyl)- N-[4-(4- isopropyl-1H- pyrazol-1-yl)-3- (1H-tetrazol-5- yl)phenyl]cyclopro- panecarboxa- mide | ¹H NMR (250 MHz, DMSO-d6) δ 1.10-1.24 (m, 8H), 1.44-1.57 (m, 2H), 2.78 (m, 1H), 7.29-7.51 (m, 5H), 7.55-7.73 (m, 2H), 7.83- 8.04 (m, 2H), 9.52 (s, 1H). LCMS (Analytical Method F): Rt = 3.68 mins; MS (ESIpos) = 448.1 (M + H)⁺. |
| 397 | 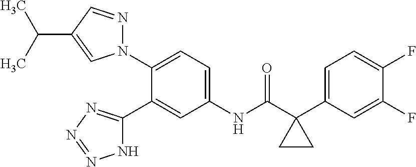 [SMILES: CC(C)c1cnn(-c2ccc(NC(=O)C3(c4ccc(F)c(F)c4)CC3)cc2-c2nnn[nH]2)c1] | 1-(3,4- difluorophenyl)- N-[4-(4- isopropyl-1H- pyrazol-1-yl)-3- (1H-tetrazol-5- yl)phenyl]cyclopro- panecarboxa- mide | ¹H NMR (250 MHz, DMSO-d6) δ 1.09-1.23 (m, 8H), 1.41-1.58 (m, 2H), 2.69-2.87 (m, 1H), 7.22- 7.34 (m, 1H), 7.34-7.44 (m, 2H), 7.45-7.56 (m, 1H), 7.56- 7.63 (m, 1H), 7.63-7.71 (m, 1H), 7.82-7.99 (m, 2H), 9.29 (s, 1H). LCMS (Analytical Method F): Rt = 3.56 mins; MS (ESIpos) m/z = 450.1 (M + H)⁺. |
| 398 | 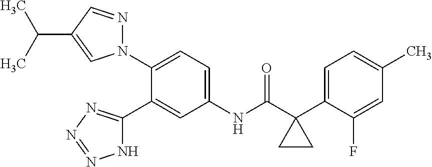 [SMILES: Cc1ccc(C2(C(=O)Nc3ccc(-n4cc(C(C)C)cn4)c(-c4nnn[nH]4)c3)CC2)c(F)c1] | 1-(2-fluoro-4- methylphenyl)- N-[4-(4- isopropyl-1H- pyrazol-1-yl)-3- (1H-tetrazol-5- yl)phenyl]cyclopro- panecarboxa- mide | ¹H NMR (250 MHz, DMSO-d6) δ 1.00-1.24 (m, 8H), 1.49-1.65 (m, 2H), 2.33 (s, 3H), 2.68-2.84 (m, 1H), 6.97-7.15 (m, 2H), 7.26- 7.45 (m, 2H), 7.52-7.75 (m, 2H), 7.83-8.01 (m, 2H), 9.11 (s, 1H). LCMS (Analytical Method D): Rt = 4.46 mins; MS (ESIpos) m/z = 446.45 (M + H)⁺. |
| 399 | 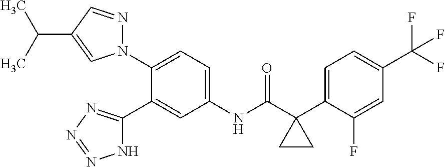 [SMILES: CC(C)c1cnn(-c2ccc(NC(=O)C3(c4ccc(C(F)(F)F)cc4F)CC3)cc2-c2nnn[nH]2)c1] | 1-[2-fluoro-4- (trifluoromethyl) phenyl]-N-[4- (4-isopropyl-1H- pyrazol-1-yl)-3- (1H-tetrazol-5- yl)phenyl]cyclopro- panecarboxa- mide | ¹H NMR (250 MHz, DMSO-d6) δ 1.15 (d, J = 6.9 Hz, 6H), 1.18- 1.26 (m, 2H), 1.62 (m, 2H), 2.63- 2.87 (m, 1H), 7.16-7.47 (m, 3H), 7.53-7.73 (m, 3H), 7.79-7.97 (m, 2H), 9.24 (s, 1H). LCMS (Analytical Method D): Rt = 4.65 mins, MS (ESIpos) m/z = 516.05 (M + H)⁺. |
| 400 | 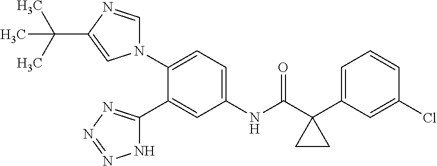 [SMILES: CC(C)(C)c1cn(-c2ccc(NC(=O)C3(c4cccc(Cl)c4)CC3)cc2-c2nnn[nH]2)cn1] | N-[4-(4-tert- butyl-1H- imidazol-1-yl)- 3-(1H-tetrazol- 5-yl)phenyl]-1- (3- chlorophenyl)cy- clopropanecarbox- amide | ¹H NMR (500 MHz, DMSO-d6) δ 9.58 (s, 1H), 8.38-8.21 (m, 1H), 8.18-8.06 (m, 1H), 7.92-7.79 (m, 1H), 7.51-7.42 (m, 2H), 7.42- 7.31 (m, 3H), 7.08-6.93 (m, 1H), 1.53-1.47 (m, 2H), 1.23 (s, 9H), 1.21-1.16 (m, 2H). LCMS (Analytical Method F): Rt = 2.61 mins, MS (ESIpos) m/z = 462 (M + H)⁺. |
| 401 | 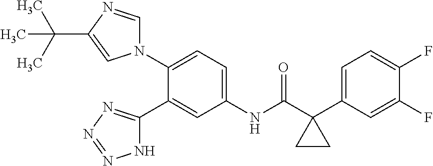 [SMILES: CC(C)(C)c1cn(-c2ccc(NC(=O)C3(c4ccc(F)c(F)c4)CC3)cc2-c2nnn[nH]2)cn1] | N-[4-(4-tert- butyl-1H- imidazol-1-yl)- 3-(1H-tetrazol- 5-yl)phenyl]-1- (3,4- difluorophenyl)cy- clopropanecar- boxamide | ¹H NMR (500 MHz, DMSO-d6) δ 9.31 (s, 1H), 8.13-7.90 (m, 2H), 7.86-7.74 (m, 1H), 7.54-7.45 (m, 1H), 7.45-7.35 (m, 2H), 7.32- 7.23 (m, 1H), 6.99-6.88 (m, 1H), 1.53-1.47 (m, 2H), 1.21 (s, 9H), 1.19-1.14 (m, 2H). LCMS (Analytical Method F): Rt = 2.52 mins, MS (ESIpos): m/z = 464 (M + H)⁺. |
| 402 | 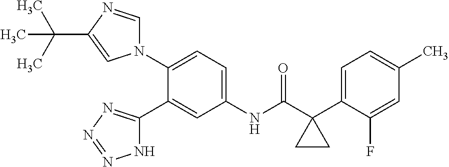 [SMILES: Cc1ccc(C2(C(=O)Nc3ccc(-n4cnc(C(C)(C)C)c4)c(-c4nnn[nH]4)c3)CC2)c(F)c1] | N-[4-(4-tert- butyl-1H- imidazol-1-yl)- 3-(1H-tetrazol- 5-yl)phenyl]-1- (2-fluoro-4- methylphenyl)cy- clopropanecar- boxamide | ¹H NMR (500 MHz, DMSO-d6) δ 9.12 (s, 1H), 8.33-8.14 (m, 1H), 8.09-8.00 (m, 1H), 7.84 (s, 1H), 7.48-7.40 (m, 1H), 7.38-7.30 (m, 1H), 7.09-6.95 (m, 3H), 2.34 (s, 3H), 1.60-1.53 (m, 2H), 1.22 (s, 9H), 1.15-1.08 (m, 2H). LCMS (Analytical Method F): Rt = 2.59 mins, MS (ESIpos): m/z = 460 (M + H)⁺. |
| 403 | 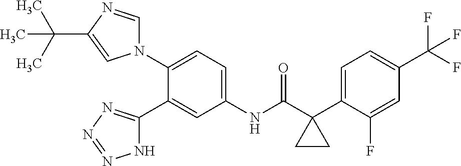 [SMILES: CC(C)(C)c1cn(-c2ccc(NC(=O)C3(c4ccc(C(F)(F)F)cc4F)CC3)cc2-c2nnn[nH]2)cn1] | N-[4-(4-tert- butyl-1H- imidazol-1-yl)- 3-(1H-tetrazol- 5-yl)phenyl]-1- [2-fluoro-4- (trifluoromethyl) phenyl]cyclopro- panecarboxamide | ¹H NMR (500 MHz, DMSO-d6) δ 9.27 (s, 1H), 8.17-7.90 (m, 2H), 7.83-7.75 (m, 1H), 7.75-7.63 (m, 2H), 7.63-7.57 (m, 1H), 7.43- 7.37 (m, 1H), 7.01-6.89 (m, 1H), 1.70-1.60 (m, 2H), 1.31- 1.14 (m, 11H). LCMS (Analytical Method F): Rt = 2.79 mins, MS (ESIpos): m/z = 514 (M + H)⁺. |
| 404 | 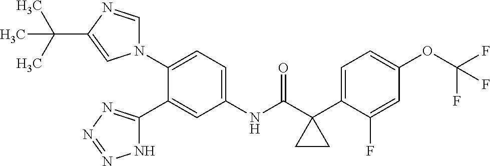 [SMILES: CC(C)(C)c1cn(-c2ccc(NC(=O)C3(c4ccc(OC(F)(F)F)cc4F)CC3)cc2-c2nnn[nH]2)cn1] | N-[4-(4-tert- butyl-1H- imidazol-1-yl)- 3-(1H-tetrazol- 5-yl)phenyl]-1- [2-fluoro-4- (trifluoromethoxy) phenyl]cyclo- propanecarboxa- mide | ¹H NMR (500 MHz, DMSO-d6) δ 9.36-9.23 (m, 1H), 8.42-8.23 (m, 1H), 8.11-8.03 (m, 1H), 7.88- 7.80 (m, 1H), 7.65-7.57 (m, 1H), 7.50-7.43 (m, 1H), 7.42- 7.35 (m, 1H), 7.29-7.22 (m, 1H), 7.11-6.95 (m, 1H), 1.71-1.54 (m, 2H), 1.29-1.16 (m, 11H). LCMS (Analytical Method F): Rt = 2.86 mins, MS (ESIpos): m/z = 530 (M + H)⁺. |
| 405 | 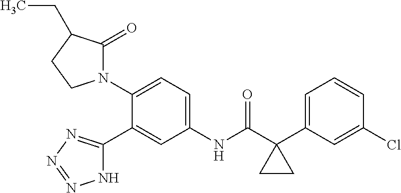 [SMILES: CCC1CCN(c2ccc(NC(=O)C3(c4cccc(Cl)c4)CC3)cc2-c2nnn[nH]2)C1=O] | 1-(3- chlorophenyl)- N-[4-(3-ethyl-2- oxopyrrolidin-1- yl)-3-(1H- tetrazol-5- yl)phenyl]cyclopro- panecarboxa- mide | ¹H NMR (500 MHz, DMSO-d6) δ 9.48 (s, 1H), 8.01 (s, 1H), 7.79 (dd, J = 8.7, 2.5 Hz, 1H), 7.45- 7.41 (m, 1H), 7.41-7.33 (m, 4H), 3.79-3.71 (m, 1H), 3.67-3.59 (m, 1H), 2.30-2.16 (m, 2H), 1.86- 1.72 (m, 1H), 1.61-1.50 (m, 1H), 1.50-1.43 (m, 2H), 1.30- 1.21 (m, 1H), 1.21-1.14 (m, 2H), 0.82 (t, J = 7.4 Hz, 3H). LCMS (Analytical Method F): Rt = 3.25 mins; MS (ESIpos): m/z = |
| | | | 451.2 (M + H)⁺. |
| 406 | 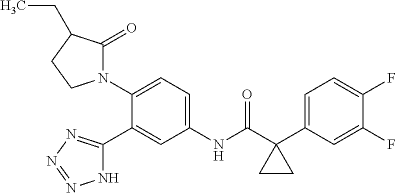 [SMILES: CCC1CCN(c2ccc(NC(=O)C3(c4ccc(F)c(F)c4)CC3)cc2-c2nnn[nH]2)C1=O] | 1-(3,4- difluorophenyl)- N-[4-(3-ethyl-2- oxopyrrolidin-1- yl)-3-(1H- tetrazol-5- yl)phenyl]cyclopro- panecarboxa- mide | ¹H NMR (500 MHz, DMSO-d6) δ 9.27 (s, 1H), 7.99 (s, 1H), 7.78 (dd, J = 8.7, 2.4 Hz, 1H), 7.49 (m, 1H), 7.45-7.36 (m, 2H), 7.29- 7.24 (m, 1H), 3.79-3.71 (m, 1H), 3.67-3.59 (m, 1H), 2.31-2.16 (m, 2H), 1.87-1.72 (m, 1H), 1.60- 1.51 (m, 1H), 1.51-1.44 (m, 2H), 1.30-1.20 (m, 1H), 1.20- 1.13 (m, 2H), 0.83 (t, J = 7.4 Hz, 3H). LCMS (Analytical Method F): Rt = |
| | | | 3.52 mins; MS (ESIpos): m/z = |
| | | | 453.2 (M + H)⁺. |
| 407 | 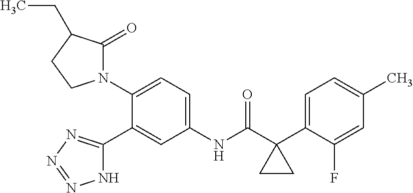 [SMILES: CCC1CCN(c2ccc(NC(=O)C3(c4ccc(C)cc4F)CC3)cc2-c2nnn[nH]2)C1=O] | N-[4-(3-ethyl-2- oxopyrrolidin-1- yl)-3-(1H- tetrazol-5- yl)phenyl]-1-(2- fluoro-4- methylphenyl)cy- clopropanecar- boxamide | ¹H NMR (500 MHz, DMSO-d6) δ 9.04 (s, 1H), 7.96 (s, 1H), 7.77 (dd, J = 8.7, 2.5 Hz, 1H), 7.40- 7.30 (m, 2H), 7.06-7.03 (m, 1H), 7.02 (s, 1H), 3.80-3.71 (m, 1H), 3.67-3.59 (m, 1H), 2.33 (s, 3H), 2.29-2.19 (m, 2H), 1.86-1.72 (m, 1H), 1.63-1.48 (m, 3H), 1.32- 1.19 (m, 1H), 1.17-1.06 (m, 2H), 0.83 (t, J = 7.4 Hz, 3H). LCMS (Analytical Method F): Rt = 3.20 mins; MS (ESIpos): m/z = |
| | | | 449.2 (M + H)⁺. |
| 408 | 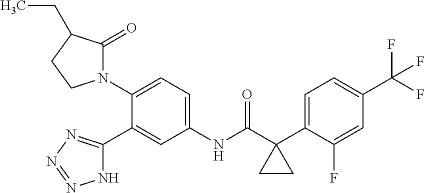 [SMILES: CCC1CCN(c2ccc(NC(=O)C3(c4ccc(C(F)(F)F)cc4F)CC3)cc2-c2nnn[nH]2)C1=O] | N-[4-(3-ethyl-2- oxopyrrolidin-1- yl)-3-(1H- tetrazol-5- yl)phenyl]-1-[2- fluoro-4- (trifluoromethyl) phenyl]cyclopro- panecarboxamide | ¹H NMR (500 MHz, DMSO-d6) δ 9.23 (s, 1H), 7.96 (s, 1H), 7.76 (dd, J = 8.7, 2.5 Hz, 1H), 7.73- 7.65 (m, 2H), 7.63-7.58 (m, 1H), 7.39 (d, J = 8.7 Hz, 1H), 3.83- 3.70 (m, 1H), 3.68-3.56 (m, 1H), 2.31-2.17 (m, 2H), 1.87-1.71 (m, 1H), 1.69-1.61 (m, 2H), 1.60- 1.50 (m, 1H), 1.32-1.18 (m, 3H), 0.83 (t, J = 7.4 Hz, 3H). LCMS (Analytical Method F): Rt = 3.42 mins; MS (ESIpos): m/z = |
| | | | 503.2 (M + H)⁺. |
| 409 | 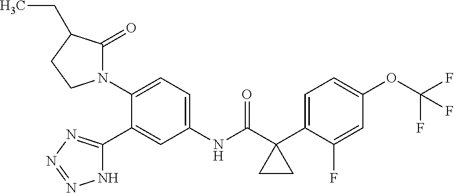 [SMILES: CCC1CCN(c2ccc(NC(=O)C3(c4ccc(OC(F)(F)F)cc4F)CC3)cc2-c2nnn[nH]2)C1=O] | N-[4-(3-ethyl-2- oxopyrrolidin-1- yl)-3-(1H- tetrazol-5- yl)phenyl]-1-[2- fluoro-4- (trifluoromethoxy) phenyl]cyclo- propanecarboxa- mide | ¹H NMR (500 MHz, DMSO-d6) δ 9.19 (s, 1H), 7.96 (s, 1H), 7.76 (dd, J = 8.7, 2.4 Hz, 1H), 7.64- 7.56 (m, 1H), 7.44-7.33 (m, 2H), 7.27-7.21 (m, 1H), 3.81-3.70 (m, 1H), 3.69-3.57 (m, 1H), 2.32- 2.17 (m, 2H), 1.86-1.72 (m, 1H), 1.66-1.49 (m, 3H), 1.33- 1.13 (m, 3H), 0.83 (t, J = 7.4 Hz, 3H). LCMS (Analytical Method F): Rt = 3.50 mins; MS (ESIpos): m/z = |
| | | | 519.2 (M + H)⁺. |
| 410 | 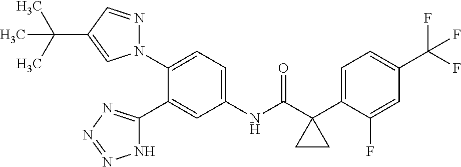 [SMILES: CC(C)(C)c1cnn(-c2ccc(NC(=O)C3(c4ccc(C(F)(F)F)cc4F)CC3)cc2-c2nnn[nH]2)c1] | 1-[2-fluoro-4- (trifluoromethyl) phenyl]-N-[4- (4-tert-butyl- 1H-pyrazol-1- yl)-3-(1H- tetrazol-5- yl)phenyl]cyclopro- panecarboxa- mide | ¹H NMR (500 MHz, DMSO-d6) δ 9.27 (s, 1H), 7.94-7.80 (m, 2H), 7.75-7.65 (m, 3H), 7.64-7.57 (m, 2H), 7.43 (s, 1H), 1.69-1.62 (m, 2H), 1.28-1.23 (m, 2H), 1.19- (s, 9H). LCMS (Analytical Method F): Rt = 3.95 mins, MS (ESIpos) m/z = 514 (M + H)⁺. |
| 411 | 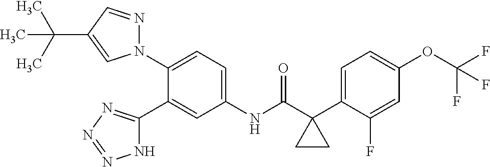 [SMILES: CC(C)(C)c1cnn(-c2ccc(NC(=O)C3(c4ccc(OC(F)(F)F)cc4F)CC3)cc2-c2nnn[nH]2)c1] | 1-[2-fluoro-4- (trifluoromethoxy) phenyl]-N-[4- (4-tert-butyl- 1H-pyrazol-1- yl)-3-(1H- tetrazol-5- yl)phenyl]cyclopro- pranecarboxa- mide | ¹H NMR (500 MHz, DMSO-d6) δ 9.24 (s, 1H), 7.94-7.80 (m, 2H), 7.71-7.55 (m, 3H), 7.43 (s, 1H), 7.38 (d, J = 8.9 Hz, 1H), 7.25 (d, J = 8.5 Hz, 1H), 1.67-1.59 (m, 2H), 1.19 (s, 11H). LCMS (Analytical Method F): Rt = 4.02 mins, MS (ESIpos) m/z = 530 (M + H)⁺. |
| 412 | 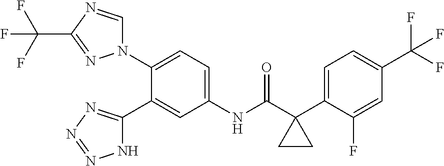 [SMILES: O=C(Nc1ccc(-n2cnc(C(F)(F)F)n2)c(-c2nnn[nH]2)c1)C1(c2ccc(C(F)(F)F)cc2F)CC1] | 1-[2-fluoro-4- (trifluoromethyl) phenyl]-N-{3- (1H-tetrazol-5- yl)-4-[3- (trifluoromethyl)- 1H-1,2,4- triazol-1- yl]phenyl}cyclopro- panecarboxa- mide | ¹H NMR (400 MHz, DMSO-d6) δ [ppm] = 1.26-1.29 (m, 2H), 1.67- 1.70 (m, 2H), 7.91 (dd, 1H), 7.61-7.75 (m, 4H), 8.27 (d, 1H), 9.03 (s, 1H), 9.45 (s, 1H). LCMS (method 1): Rt = 1.24 min; MS (ESIpos) m/z = 527 (M + H)⁺. |
| 413 | 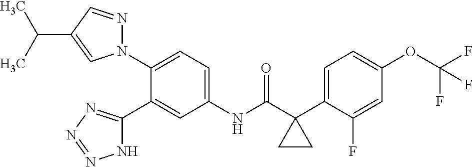 [SMILES: CC(C)c1cnn(-c2ccc(NC(=O)C3(c4ccc(OC(F)(F)F)cc4F)CC3)cc2-c2nnn[nH]2)c1] | 1-[2-fluoro-4- (trifluoromethoxy) phenyl]-N-[4- (4-isopropyl-1H- pyrazol-1-yl)-3- (1H-tetrazol-5- yl)phenyl]cyclopro- panecarboxa- mide | ¹H NMR (250 MHz, DMSO-d6) δ 1.15 (d, J = 6.9 Hz, 6H), 1.18- 1.26 (m, 2H), 1.62 (m, 2H), 2.63- 2.87 (m, 1H), 7.16-7.47 (m, 3H), 7.53-7.73 (m, 3H), 7.79-7.97 (m, 2H), 9.24 (s, 1H). LCMS (Analytical Method D): Rt = 4.65 mins, MS (ESIpos) m/z = 516.05 (M + H)⁺. |

### Biological Assays

Examples were tested in selected biological assays one or more times. When tested more than once, data are reported as either average values or as median values, wherein
The average value, also referred to as the arithmetic mean value, represents the sum of the values obtained divided by the number of times tested, and
The median value represents the middle number of the group of values when ranked in ascending or descending order. If the number of values in the data set is odd, the median is the middle value. If the number of values in the data set is even, the median is the arithmetic mean of the two middle values.

Examples were synthesised one or more times. When synthesised more than once, data from biological assays represent average values or median values calculated utilising data sets obtained from testing of one or more synthetic batch.

The potency to inhibit the Bradykinin B1 receptor was determined for the compounds of this invention in a cell-based fluorescent calcium-mobilisation assay. The assay measures the ability of test compounds to inhibit Bradykinin B1 receptor agonist-induced increase of intracellular free Ca² in the cell line expressing Bradykinin B1 receptor. Specifically, calcium indicator-loaded cells are pre-incubated in the absence or presence of different concentrations of test compounds followed by the stimulation with a selective Bradykinin B1 receptor agonist peptide. The change of the intracellular Ca² concentration is monitored with a fluorescent plate reader FLIPR TETRA® (Molecular Devices).

Calcium flux Assays (FLIPR) with cells expressing human Bradykinin B1 receptor (hB1)

Calcium flux Assay (FLIPR) with recombinant cells for Bradykinin B1 receptor antagonist, either in the presence (hB1 IC₅₀) or absence (hB1 free IC₅₀) of 0.1% Bovine Serum Albumin (BSA) in assay buffer.

CHO-K1 cell line expressing human Bradykinin B1 receptor was purchased from Euroscreen (Gosselies, Belgium, with reference name hB1-D1). The cells were grown in Nutrient Mixture Ham's F12 (Sigma) containing 10% Foetal bovine serum (Sigma) and 400 μg/mL G418 (Sigma), 5 μg/mL puromycim (Sigma).

Notably, compound examples were tested in the FLIPR assays either in the presence (hB1 IC₅₀) or absence (hB1 free IC₅₀) of 0.1% BSA in assay buffer, in order to assess the potency shifts due to serum protein binding of compound examples. The effect of BSA on the potency of endothelin receptor antagonists have been described in the prior art (Wu-Wong, J. R. et al. (1997), JPET 281: 791-798). The teaching can be applied in analogy to testing the potency of Bradykinin B1 receptor antagonist in the FLIPR assays.

For the calcium flux assay, 80% confluent cells were detached from the culture vessels with Versene (Gibco), and seeded into 384-well plates (Cell binding Surface; Corning, N.Y.; #3683) at a density of 15,000 cells per well. Cells were seeded in a volume of 50 μL in medium without antibiotics and incubated overnight in a humidified atmosphere with 5% CO₂ at 37° C. The following day, the medium was replaced with 20 μL of 5 μM Fluo-4AM dye (Molecular Probes) in assay buffer (2.5 mM probenicid, 1 mg/mL pluronic acid, 135 mM NaCl, 5 mM KCl, 1.8 mM CaCl, 1 mM MgCl₂, 10 mM HEPES, 5.6 mM glucose, and 0.05% gelatine, pH 7.4), which contains or lacks 0.1% BSA for determination of compound potency units as hB1 IC₅₀ or hB1 free IC₅₀, respectively. The calcium indicator loaded cells were incubated at 37° C. for 2 hrs. Extracellular dye was then removed and each well was filled with 45 μL of assay buffer. Cell plates were kept in dark until used. Compound examples were assayed at 8 concentrations in triplicate. Serial 10-fold dilutions in 100% DMSO were made at a 100-times higher concentration than the final concentration, and then diluted 1:10 in assay buffer. 5 μL of each diluted compound was added to the well of cell plates (yielding final concentration with 1% DMSO), and incubated for 30 min at 28° C. before the addition of Bradykinin B1 receptor agonist on the FLIPR instrument.

Agonist plates contained the agonist Lys-(Des-Arg)-Bradykinin (Bachem, Brackley) at 3.5×EC₉₀ in assay buffer with 1% DMSO. The addition of agonist 20 μl per well to the assay plate was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 □M was used to determine the full inhibition as control.

Peak fluorescence was used to determine the response to agonist obtained at each concentration of compound examples by the following equation:

% Response=100*(RFU_{(compound)}−RFU_{(control)})/(RFU_{(DMSO)}−RFU_{(control)})
RFU means relative fluorescence units.
Control means full inhibition by the peptide antagonist Lys-(Des-Arg-Leu)-Bradykinin at 20 □M.

The response values were plotted against the logarithm of the compound concentrations. The compound examples were tested in triplicates per plate and mean values were plotted in Excel XLfit to determine IC₅₀ values, percentage of maximal inhibition and the Hill slopes.

Calcium flux Assay (FLIPR) with human fibroblasts expressing Bradykinin B1 receptor (hB1 IMR-90)

The Calcium flux Assay was carried out utilising IMR-90 human foetal lung fibroblasts (American Type Culture Collection, Rockville, Md.; and Coriell Institute, Camden, N.J.), which express native human Bradykinin B1 receptor after induction with human IL-10.

The fibroblasts were cultured in complete growth media comprised of Dulbecco's modified Eagle's medium (DMEM; Sigma) containing 10% foetal bovine serum, 4 mM L-glutamine, and 1% nonessential amino acids. The cells were maintained in a humidified atmosphere with 5% CO₂ at 37° C. and were sub-cultured at a ratio of 1:3, every other day.

For the assay, IMR-90 fibroblasts were harvested using TrypLE Express (GIBCO/Invitrogen) and seeded into 384-well plates (Corning Cellbinding Surface, Cat. 3683) at a density of 15000 cells/well. The following day, cells were treated with 0.35 ng/mL human IL-10 in 10% FBS/MEM for 3h to up-regulate Bradykinin B1 receptor. Induced cells were loaded with fluorescent calcium indicator by incubation with 2.5 μM Fluo-4/AM (Invitrogen) at 37° C., 5% CO₂ for 2 h in the presence of 2.5 mM probenecid in 1% FBS/MEM. Extracellular dye was removed by washing with assay buffer (2.5 mM probenecid and 0.1% BSA in 20 mM HEPES/HBSS without bicarbonate or phenol red, pH 7.5). Compound examples were assayed at 8 concentrations in triplicate. After addition of compound examples to the cell plate and incubation for 30 min at 28° C., the addition of Bradykinin B1 agonist Lys-(Des-Arg)-Bradykinin (Bachem, Brackley) at a final concentration of EC₉₀ was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 □M was used to determine the full inhibition as control. IC₅₀ values were determined by the same way described for the FLIPR assay with recombinant cells.

TABLE 1
IC₅₀ values of compound examples in Calcium flux
Assay (FLIPR) (Blank spaces indicate that the appropriate
IC₅₀ value has not been determined)

| Example | hB1 | hB1 free | hB1 IMR-90 |
|---|---|---|---|
| Number | IC₅₀ [nM] | IC₅₀ [nM] | IC₅₀ [nM] |
| 1 | 22.6 | 3.35 | 14.2 |
| 2 | 58.9 | | |
| 3 | 54.8 | | |
| 4 | 158 | | |
| 5 | 1510 | | |
| 6 | 1370 | | |
| 7 | 1350 | | |
| 8 | 347 | 337 | |
| 9 | 660 | | |
| 10 | 68.8 | | |
| 11 | 43.0 | | |
| 12 | 18.1 | 56.4 | 39.3 |
| 13 | 86.6 | | |
| 14 | 101 | | |
| 15 | 40.9 | | |
| 16 | 31.7 | | 528 |
| 17 | 68.5 | | 263 |
| 18 | 40.2 | | 27.4 |
| 19 | 42.9 | | |
| 20 | 54.4 | 29.2 | |
| 21 | 367 | | |
| 22 | 212 | | |
| 23 | 176 | 145 | 35.2 |
| 24 | 232 | | |
| 25 | 57.4 | 25.9 | |
| 26 | 91.7 | | |
| 27 | 51.5 | | |
| 28 | 138 | | |
| 29 | 167 | | |
| 30 | 201 | | |
| 31 | 212 | | |
| 32 | 41.6 | | |
| 33 | 238 | 284 | |
| 34 | 603 | | |
| 35 | 213 | | |
| 36 | 128 | | 104 |
| 37 | 1830 | | |
| 38 | 38.8 | 13.7 | |
| 39 | 80.3 | | |
| 40 | 31.4 | | |
| 41 | 92.0 | | |
| 42 | 746 | | |
| 43 | 14.0 | | |
| 44 | 23.0 | 5.70 | 5.47 |
| 45 | 49.8 | | |
| 46 | 141 | | |
| 47 | 180 | | |
| 48 | 50.5 | | 29.3 |
| 49 | 213 | | |
| 50 | 310 | | |
| 51 | 342 | | |
| 52 | 122 | | |
| 53 | 514 | | |
| 54 | 544 | | |
| 55 | 2060 | | |
| 56 | 21.7 | 15.3 | 20.2 |
| 57 | 36.6 | | |
| 61 | 2050 | | |
| 62 | 2430 | | |
| 67 | 394 | | |
| 68 | 1410 | | |
| 69 | 30.9 | 6.75 | 16.2 |
| 70 | 12.5 | 9.27 | 6.00 |
| 71 | 15.1 | 22.0 | |
| 72 | 15.5 | 6.82 | 5.72 |
| 73 | 16.2 | 13.9 | 7.98 |
| 74 | 18.7 | 7.76 | |
| 75 | 22.7 | 14.1 | |
| 76 | 24.0 | 12.0 | |
| 77 | 24.0 | 16.1 | 21.7 |
| 78 | 24.5 | 21.2 | 15.8 |
| 79 | 25.1 | | 24.7 |
| 80 | 25.6 | 29.6 | 27.2 |
| 81 | 27.5 | 29.1 | 21.2 |
| 82 | 33.3 | 29.1 | 14.9 |
| 83 | 37.5 | 60.5 | 33.0 |
| 84 | 41.8 | 19.0 | |
| 85 | 63.1 | | |
| 86 | 92.5 | 40.3 | 27.4 |
| 87 | 88.0 | 60.0 | |
| 88 | 111 | | |
| 89 | 615 | 541 | |
| 90 | 1560 | | |
| 91 | 174 | 452 | 161 |
| 92 | 39.8 | 37.7 | 25.3 |
| 93 | 37.1 | 39.3 | |
| 94 | 23.8 | | 25.1 |
| 95 | 14.5 | 10.4 | |
| 96 | 6.58 | 5.32 | 15.3 |
| 97 | 138 | | 101 |
| 98 | 8.67 | | 5.78 |
| 99 | 24.0 | | 11.8 |
| 100 | 20.2 | 5.56 | 8.84 |
| 101 | 38.7 | 24.5 | 22.1 |
| 102 | 34.8 | | 21.0 |
| 103 | 28.1 | | |
| 104 | 46.4 | | |
| 105 | 10.9 | 4.54 | 10.7 |
| 106 | 56.0 | | |
| 107 | 134 | 177 | |
| 108 | 338 | | |
| 109 | 12.2 | | |
| 110 | 12.7 | 5.68 | 11.0 |
| 111 | 16.6 | 4.98 | 10.6 |
| 112 | 44.9 | 16.2 | 20.4 |
| 113 | 1200 | | |
| 114 | 1350 | | |
| 115 | 169 | | |
| 116 | 177 | | |
| 117 | 448 | | |
| 118 | 1820 | | |
| 119 | 22.7 | 2.88 | 13.1 |
| 120 | 30.6 | | 25.1 |
| 121 | 28.2 | | 19.4 |
| 122 | 56.5 | | 28.7 |
| 123 | 645 | | |
| 124 | 38.3 | | |
| 125 | 13.8 | 23.3 | 26.0 |
| 126 | 21.9 | | 13.1 |
| 127 | 90.9 | 88.6 | 27.9 |
| 128 | 137 | 2.68 | 63.3 |
| 129 | | 77.2 | |
| 130 | | 362 | |
| 131 | | 53.0 | |
| 132 | | 234 | |
| 133 | | 217 | |
| 134 | | 572 | |
| 135 | 516 | 58.7 | 91.8 |
| 136 | | 165 | 15.7 |
| 137 | | 17.1 | 43.9 |
| 138 | | 51.3 | 102 |
| 139 | | 22.6 | 91.0 |
| 140 | | 42.1 | 85.1 |
| 141 | | 224 | 141 |
| 142 | | 201 | |
| 143 | | 144 | |
| 144 | | 300 | 289 |
| 145 | | 194 | |
| 146 | | 100 | |
| 147 | | 35.3 | 36.3 |
| 148 | 20.0 | 1.94 | 18.5 |
| 149 | 14.4 | 6.35 | 11.8 |
| 150 | 39.2 | | |
| 151 | 127 | | |
| 152 | 23.5 | | |
| 153 | 26.2 | 3.64 | 18.6 |
| 154 | 27.1 | | |
| 155 | 220 | | |
| 156 | 27.5 | 36.6 | 84.6 |
| 157 | 140 | | |
| 158 | 764 | | |
| 159 | 585 | 40.3 | |
| 160 | 47.7 | 8.13 | 26.7 |
| 161 | 24.9 | | |
| 162 | 59.0 | | |
| 163 | 28.1 | 14.2 | 28.9 |
| 164 | 14.5 | | |
| 165 | 64.1 | | |
| 166 | 2160 | 1130 | |
| 167 | | 634 | |
| 168 | 88.1 | 58.6 | 27.7 |
| 169 | 117 | | |
| 170 | | 968 | |
| 171 | 358 | 422 | 281 |
| 172 | 347 | 433 | |
| 173 | | 86.5 | 92.4 |
| 174 | | 1110 | |
| 175 | | 216 | 687 |
| 176 | | 258 | |
| 177 | | 274 | |
| 178 | | 287 | |
| 179 | | 393 | |
| 180 | | 768 | |
| 181 | 14.8 | 20.8 | 7.29 |
| 182 | 1350 | 185 | |
| 183 | 33.0 | 54.4 | 11.1 |
| 184 | 50.5 | 24.6 | 22.3 |
| 185 | | 251 | |
| 186 | | 252 | |
| 187 | | 1620 | |
| 189 | | 2350 | |
| 190 | 250 | 189 | 340 |
| 191 | | 11.8 | 20.5 |
| 192 | | 33.9 | 29.1 |
| 193 | | 95.3 | 80.6 |
| 194 | 575 | 569 | |
| 195 | 311 | 183 | |
| 196 | 1370 | 759 | 1450 |
| 197 | 993 | 493 | |
| 198 | | 943 | |
| 199 | 2400 | 1580 | |
| 200 | | 1170 | |
| 201 | | 374 | |
| 202 | | 83.2 | 50.9 |
| 203 | | 92.2 | 36.7 |
| 204 | | 125 | |
| 205 | | 170 | |
| 206 | | 485 | |
| 207 | | 22.4 | 14.9 |
| 208 | 15.8 | 22.6 | 11.2 |
| 209 | | 2.96 | 3.05 |
| 210 | 78.0 | 131 | |
| 211 | 23.1 | 10.5 | 19.1 |
| 212 | | 2.77 | 3.92 |
| 213 | 63.2 | 18.0 | 24.7 |
| 214 | 47.2 | 7.93 | 26.9 |
| 215 | | 3.21 | 8.48 |
| 216 | | 40.6 | 38.8 |
| 217 | | 516 | |
| 218 | 75.0 | 264 | 31.6 |
| 219 | | 86.8 | |
| 220 | 49.8 | 27.4 | 23.1 |
| 221 | 160 | | |
| 222 | 75.7 | | 55.6 |
| 223 | 84.0 | | |
| 224 | 49.6 | | 108 |
| 225 | 88.9 | | |
| 226 | 105 | | |
| 227 | 83.8 | | 177 |
| 228 | 88.2 | | |
| 229 | 1280 | | |
| 230 | 2100 | | |
| 231 | 2200 | | |
| 232 | 18.6 | 8.26 | 6.80 |
| 233 | 68.6 | 18.9 | 35.2 |
| 234 | 29.9 | 11.0 | 18.6 |
| 235 | 53.6 | | |
| 236 | | 2.90 | 15.6 |
| 240 | | 2.35 | 2.83 |
| 241 | | 54.2 | 127 |
| 242 | | 9.76 | 22.8 |
| 243 | | 203 | 575 |
| 244 | | 176 | 512 |
| 245 | | 8.11 | 7.85 |
| 246 | | 16.0 | |
| 247 | 61.8 | 20.0 | 37.3 |
| 248 | | 14.4 | 25.2 |
| 249 | 1760 | | |
| 250 | 240 | | |
| 251 | | 227 | |
| 252 | | 92.8 | |
| 253 | 33.5 | 16.1 | 14.9 |
| 254 | 245 | 309 | |
| 255 | 452 | 702 | |
| 256 | 274 | 288 | |
| 257 | 916 | | |
| 258 | | 35.2 | 47.3 |
| 259 | | 3.52 | 10.6 |
| 260 | | 18.8 | 14.9 |
| 261 | | 212 | 204 |
| 262 | 72.3 | | |
| 263 | 34.5 | 7.72 | 33.8 |
| 264 | 834 | 2330 | |
| 265 | | 2.63 | 2.28 |
| 266 | | 3.45 | 2.38 |
| 267 | | 19.0 | 48.3 |
| 268 | | 92.0 | |
| 269 | | 13.2 | |
| 270 | | 17.4 | |
| 271 | | 6.09 | |
| 272 | | 4.72 | |
| 273 | | 24.9 | |
| 274 | | 6.35 | |
| 275 | | 8.48 | |
| 276 | | 1.83 | |
| 277 | | 3.19 | |
| 278 | | 3.62 | |
| 279 | | 23.1 | 16.3 |
| 280 | | 11.3 | |
| 281 | | 6.00 | 6.17 |
| 282 | | 3.49 | |
| 283 | | 8.19 | |
| 284 | | 15.3 | |
| 285 | | 15.8 | |
| 286 | | 4.34 | |
| 287 | | 3.93 | |
| 288 | | 6.81 | |
| 289 | | 50.2 | |
| 290 | | 135 | |
| 291 | | 22.5 | 2.36 |
| 292 | | 7.85 | |
| 293 | | 297 | 266 |
| 294 | | 5.96 | 6.13 |
| 295 | | 24.1 | 33.8 |
| 296 | | 1730 | |
| 297 | | 350 | |
| 298 | | 368 | |
| 299 | | 241 | |
| 300 | | 58.1 | |
| 301 | | 36.1 | |
| 302 | | 44.0 | |
| 303 | | 52.0 | |
| 304 | | 18.5 | |
| 306 | 133 | 45.7 | |
| 309 | | 587 | |
| 313 | 191 | | |
| 314 | 340 | | |
| 315 | 235 | | |
| 316 | 328 | | |
| 317 | 52.9 | | |
| 318 | 59.1 | | |
| 319 | 521 | 436 | |
| 320 | 208 | | |
| 321 | 152 | | |
| 322 | 238 | | |
| 323 | 355 | | |
| 325 | 1740 | | |
| 326 | 22.3 | 20.0 | 45.2 |
| 327 | 70.9 | 71.2 | 164 |
| 328 | 77.9 | 13.5 | 127 |
| 329 | 90.5 | 85.0 | 70.3 |
| 330 | 158 | 141 | |
| 331 | 206 | 162 | |
| 332 | 274 | 191 | |
| 333 | 420 | | |
| 334 | 936 | 99.8 | |
| 335 | 517 | 298 | |
| 336 | 1320 | | |
| 337 | 2030 | | |
| 338 | 20.4 | 8.98 | 6.03 |
| 339 | 46.7 | 23.7 | |
| 340 | 161 | 265 | 108 |
| 341 | 115 | 87.3 | |
| 342 | 1770 | | |
| 343 | 113 | 259 | 110 |
| 345 | | 28.5 | |
| 346 | | 65.9 | |
| 347 | | 96.6 | |
| 348 | | 122 | |
| 349 | | 590 | |
| 350 | | 804 | |
| 351 | | 1060 | |
| 352 | | 1200 | |
| 353 | | 1210 | |
| 354 | | 1880 | |
| 355 | | 1880 | |
| 356 | | 2350 | |
| 357 | | 44.8 | |
| 358 | | 2970 | |
| 359 | | 676 | |
| 360 | | 1700 | |
| 361 | | 419 | |
| 362 | | 26.4 | |
| 363 | | 37.1 | |
| 364 | | 12.7 | |
| 365 | | 535 | |
| 366 | | 94.2 | |
| 367 | | 61.2 | |
| 368 | | 26.0 | |
| 369 | | 1130 | |
| 370 | | 873 | |
| 371 | | 282 | |
| 372 | | 31.1 | |
| 373 | | 24.2 | |
| 374 | | 1890 | |
| 375 | | 60.6 | |
| 376 | | 12.0 | |
| 377 | | 6.57 | |
| 378 | | 343 | |
| 379 | | 257 | |
| 380 | | 30.8 | |
| 381 | | 36.7 | |
| 382 | | 768 | |
| 383 | | 142 | |
| 384 | | 14.9 | |
| 385 | | 54.7 | |
| 386 | | 146 | |
| 387 | | 434 | |
| 388 | | 33.4 | |
| 389 | | 11.0 | |
| 390 | | 14.0 | |
| 391 | | 1890 | |
| 393 | | 187 | |
| 394 | | 68.1 | |
| 395 | | 16.2 | |
| 396 | | 30.7 | |
| 397 | | 38.5 | |
| 398 | | 20.6 | |
| 399 | | 5.16 | |
| 400 | | 25.4 | |
| 401 | | 34.5 | |
| 402 | | 25.3 | |
| 403 | | 20.6 | |
| 404 | | 6.36 | |
| 405 | | 1390 | |
| 406 | | 2160 | |
| 407 | | 474 | |
| 408 | | 131 | |
| 409 | | 54.7 | |
| 410 | | 6.05 | |
| 411 | | 6.84 | |
| 412 | | 504 | |
| 413 | | 5.84 | |

Inhibitory activity on Bradykinin B1 agonist-induced secretion of IL-6 and IL-8 in human IMR-90 cells

The effect of the compound examples on secretion of the cytokine IL-6 and IL-8 has been investigated in the human foetal lung fibroblast IMR-90 cell line. Here the induction of the cytokine secretion was induced by the Bradykinin B1 agonists Lys-[Des-Arg9]Bradykinin (CAS 71800-36-7, Tocris Bioscience) and Sar-[D-Phe8]-des-Arg9-Bradykinin (CAS 126959-88-4, Tocris Bioscience) leading to the activation of the Bradykinin B1-driven signalling pathway. This effect is indicative for a strong anti-inflammatory mode of action in kinin driven inflammation.

IMR-90 cells were cultured in Eagle's Minimum Essential Medium (EMEM) containing 2 mM L-glutamine, 1 g/L glucose, 1.5 g/L NaHCO₃, 1 mM sodium pyruvate and non-essential amino acids (ATCC, 30-2003™) supplemented with 10% FBS (Biochrom, S0615) and 50 U/mL Penicillin, 50 μg/mL Streptomycin (PAA, P11-010). The assay was performed in EMEM and a cell density of 5×10-4 IMR-90 cells/96-well. The compound examples have been serial diluted in 100% DMSO and evaluated at 8 different concentrations within the range of 3 nM and 10 μM and a final DMSO concentration of 0.4%. The IMR-90 cells have been incubated with the respective concentration of the compound for 30 min. The enhanced secretion of IL-6 and IL-8 was induced by the stimulation of these cells with 0.1 μM Lys-[Des-Arg9]Bradykinin (Tocris, catalogue no. 3225) and 0.1 μM Sar-[D-Phe8]-des-Arg9-Bradykinin (Tocris, catalogue no. 3230) for 5 hours at 37° C. and 5% C₀₂. Further, cells have been treated with Lys-[Des-Arg9]Bradykinin and Sar-[D-Phe8]-des-Arg9-Bradykinin as neutral control and with 0.1% DMSO as inhibitor control. The amount of IL-6 and IL-8 in the supernatant was determined using the Human Prolnflammatory Panel II (4-Plex) (MSD, K15025B) according to manufacturer's instruction. Briefly, supernatants were added onto assay plates and incubated at room temperature for 1-2 h with vigorous shaking at 600 rpm. Detection antibodies were then added onto the supernatants and incubated at room temperature for an additional 1-2 h with vigorous shaking at 600 rpm. Plates were washed three times with phosphate-buffered saline (PBS; 137 mM NaCl, 2.7 mM KCl, 6.5 mM Na2HPO4, 1.7 mM KH2P04) containing 0.05% Tween-20 (Bio-Rad, 161-0781) and electrochemiluminescence detected using the MSD Sector Imager 6000 plate reader. The cell viability was measured using the CellTiter-Glo Luminescent Assay (Promega, G7571) following the manufacturers protocol. Briefly, the CellTiter-Glo Reagent was diluted with PBS (1:1) and added directly to cells. After incubation and shaking for 10 minutes luminescent signal was measured which is proportional to the amount of ATP present.

The effect of the compound on the amount of secreted cytokine has been calculated as 100/(measured cytokine concentration of neutral control-measured cytokine concentration of inhibitor control)*(measured cytokine concentration of compound dose-measured cytokine concentration of inhibitor control). IC50 values are determined using 4-parameter-fit.

The cell viability was measured using the CellTiter-Glo Luminescent Assay (Promega, G7571) following the manufacturer's protocol. The homogeneous assay procedure involves adding the single reagent (CellTiter-Glo Reagent) directly to cells cultured in serum-supplemented medium. Cell washing, removal of medium and multiple pipetting steps are not required. The system is able to detect as few as 15 cells/well in a 384-well format in 10 minutes after adding reagent and mixing. The homogeneous add-mix-measure format results in cell lysis and generation of a luminescent signal proportional to the amount of ATP present. The amount of ATP is directly proportional to the number of cells present in culture. The CellTiter-Glo Assay generates a glow-type luminescent signal, which has a half-life generally greater than five hours, depending on cell type and medium used. The extended half-life eliminates the need to use reagent injectors and provides flexibility for continuous or batch mode processing of multiple plates. The unique homogeneous format avoids errors that may be introduced by other ATP measurement methods that require multiple steps.

The compound examples were tested in triplicates per plate and the inhibitory activity was determined as the relation between neutral and inhibitor control in percent. IC50 values were calculated using the 4-parameter logistic model.

The compound examples listed in Table 2 showed no effect on the cell viability of the stimulated IMR-90 cells. The effect on the secretion of Il-6 and IL-8 is shown in Table 2:

TABLE 2
Effect on the secretion of Il-6 and IL-8. (Blank spaces indicate that
the appropriate IC₅₀ value are >10 μM or no secretion)

| | IL-6 secretion | IL-8 secretion |
|---|---|---|
| Example | IC₅₀ (nM) | IC₅₀ (nM) |
| 1 | 225 | 60 |
| 253 | 57 | 75 |
| 380 | — | 345 |
| 404 | 69 | 59 |
| 389 | 253 | 258 |

Rat CFA in vivo model

Male Sprague Dawley rats were used. Mechanical hyperalgesia was induced by injecting 25 μL of Complete Freund's Adjuvant (CFA) into the plantar surface of one hind paw. Mechanical hyperalgesia was measured using the Pressure Application Measurement apparatus (Ugo Basile, Gemonio, Italy). Briefly, a linearly increasing pressure was applied to an area of ˜ 50 mm2 of the plantar side of the hind paw until a behavioural response (paw withdrawal) was observed or until the pressure reached 1000 gf. The pressure at which the behavioural response occurred was recorded as the “Paw Withdrawal Threshold” (PWT). Both CFA-injected and contralateral PWTs were determined for each rat, in each treatment group and for the vehicle group at each time point of the studies. Each compound example was administered orally in the vehicle solution of dimethylsulfoxide (DMSO), Polyethylenglycol (PEG) and 2-Hydroxypropyl-beta-cyclodextrin (HPCD) (v/v/v=3:20:77). Rats received a first dose as specified in Table 3 in a solution of 5 mL/kg bodyweight application volume 1 hour before CFA injection and a second dose 24 hours later. Mechanical hyperalgesia testing was performed approximately 2 hours before CFA injection, then 2 and 4 hours after the second dose of compound example (i.e. 26 and 28 hours after CFA treatment). Animals in the vehicle group were treated in the identical way like the other groups but without compound in the orally applied solution. Data were expressed as the mean±S.D. Area Under the Curve (AUC) of PWTs (defined in table 3 as “AUC of Paw withdrawal threshold (AUC 0-4 hours) post-vehicle” with respect to vehicle group or “AUC of Paw withdrawal threshold (AUC 0-4 hours) post-drug” with respect to the compound example). Data were analysed by performing a one-way ANOVA followed by a Dunnett's post hoc test. For p values less than 0.05 the results were deemed to be statistically significant.

TABLE 3
Paw withdrawal threshold after treatment
with compound example no. 253

| | AUC of Paw | | AUC of Paw |
|---|---|---|---|
| | withdrawal threshold | | withdrawal threshold |
| | (AUC 0-4 hours) | Dose, | (AUC 0-4 hours) |
| Example | post-vehicle | p.o. | post-drug |
| 253 | 1083 ± 93 gf | 3 mg/kg | 1415 ± 274 gf (*) |
| 253 | 1083 ± 93 gf | 10 mg/kg | 1439 ± 310 gf (**) |
| 253 | 1083 ± 93 gf | 30 mg/kg | 1649 ± 301 gf (***) |

(*) p < 0.05,
(**) p < 0.01,
(***) p < 0.0001, Dunnett's post-hoc test, different from vehicle group

### Rat Paw Oedema In Vivo Model

Male Sprague Dawley rats, approximately 250 g body weight, were used. The compound examples were administered orally in the vehicle as described in “Rat CFA in vivo model”, supra. Rats were treated orally (p.o.) with a solution of 5 mL/kg bodyweight of the vehicle only or 5 mL/kg bodyweight of a solution containing the vehicle plus the compound in a dose as indicated in Table 4. Rats received an intraplantar injection of IL-1B (5 μg in 20 μL) and des-Arg9-bradykinin (DABK; 10 μg in 20 μL) at 20 and 40 minutes after compound treatment, respectively. Paw oedema was measured by water displacement using a plethysmometer (Ugo Basile, Gemonio, Italy). Paw oedema measurement was performed before compound administration (baseline) and subsequently at 20, 40 and 60 minutes after DABK injection. Paw oedema was calculated by subtracting the baseline value to post-DABK treatment values for each individual and at each time point. Data were analysed by calculating the area under the paw oedema time curve for each individual (0-60 minutes post-DABK). The effect of the compound example, relative to that of the vehicle, was analysed by performing a one-way ANOVA followed by a Dunnett's post-hoc test. For p values less than 0.05 the results were deemed to be statistically significant. The mean and standard deviation were calculated for each treatment group for graphical representations.

TABLE 4
Rat paw oedema measurement after treatment
with compound example no. 253

| | | AUC of Paw Oedema |
|---|---|---|
| Example | Dose | (AUC 0-1 hour post DABK) |
| Vehicle | — | 6.45 ± 1.14 |
| 253 | 10 mg/kg, p.o. | 3.24 ± 2.73 (*) |
| 253 | 30 mg/kg, p.o. | 3.67 ± 2.35 (*) |

(*) p < 0.05, versus vehicle, Dunnett's post hoc test

### In Vivo Adjuvant-Induced Arthritis Model

To determine the anti-inflammatory activity of the compound examples, they are examined for their in vivo efficacy in an arthritis model. For this purpose, male Lewis rats (about 100-125 g, Charles River Laboratories, Germany) are each administered 100 μl of a complete Freund's adjuvant (CFA) solution (M. tuberculosis H37Ra [Difo Lab, Cat. No. −231141] dissolved in Incomplete Freund's adjuvant [Difco Lab, Cat. No. −263910]) into the tailhead subcutaneously on day 0. There are n=8 rats in each group. Both, a healthy control group and a disease control group are included in the study. Each control group is given p.o. treatment only with the vehicle (5% DMSO/95% PBS) of the compound examples. The treatment with different dosages of the compound examples dissolved in the vehicle is conducted in a preventative manner, i.e. starting from day 0, by oral administration. On day 0, the starting condition of the animals is additionally determined in terms of the disease activity scores (rating of the severity of arthritis based on a points system). Here, points are awarded according to the extent of joint inflammation from 0 to 4 for the presence of an erythema including joint swelling (0=none; 1=slight; 2=moderate; 3=distinct; 4=severe) for both hind paws and are added up. To determine the anti-inflammatory efficacy of the compound examples, the disease activity of the animals is scored by means of disease activity scoring starting from day 8, when the animals first exhibit signs of arthritis, and subsequently 3 times per week, until the end (day 20). Statistical analysis is performed using single-factor variance analysis (ANOVA) and by comparison with the control group by means of multiple comparative analysis (Dunnett's test).

The s.c. administration of CFA in rats leads to acute arthritis with distinct joint inflammation in rats. This induced arthritis is inhibited by the treatment with compound examples.

Isovolumetric Bladder contraction model (rat)

The aim of this study is to test the efficacy of compound examples on bladder function, in particularly on contraction frequency/intercontraction interval and contraction magnitude/contraction amplitude.

The experimental setup for performing the isovolumetric bladder measurements is adapted to a previous descripted protocol (Yoshiyama M, de Groat W C. Am J Physiol Regul Integr Comp Physiol 280: R1414-R1419, 2001).

Briefly, female Sprague Daley rats (˜200 g) are housed under normal conditions for laboratory rats in a 12:12-h light:dark cycle. Experiments are performed on urethane-anesthetized (1.2 g/kg i.p.) rats. For i.v. administration of compound examples a PE-50 catheter is inserted into Vena jugularis. A transurethral bladder catheter (PE-50) is connected to a 3-way tap, which is connected to a pressure transducer on one side and an infusion pump (e.g. B. Braun) on the other. The pressure transducer is connected via an amplifier (e.g. both from ADIstruments) to the data acquisition software program (LabChart, ADInstruments) and a computer to record the bladder pressure isovolumetrically with the urethral outlet ligated. The bladder is filled via the bladder catheter and the infusion pump by incremental volumes of physiological saline until spontaneous bladder contractions occure. For isovolumetric recording, the ureters are tied distally and cut. At least five cycles of isovolumetric bladder contraction are recorded before the compound examples are administered via the catheter in the Vena jugularis at different dosages. 2 minutes after compound administration the next e.g. five contraction cycles are recorded. The change in contraction amplitude and intercontraction interval of the recorded bladder pressure and bladder contractions is calculated by comparing the means before and after compound administration using e.g. GraphPad Prism 6 program.

Cyclophosphamide-induced overactive bladder (rats)

The aim of this study is to test the efficacy of compound examples on overactive bladder as well as on cystitis in cyclophosphamide-treated rats.

The experimental setup is adapted to a previous descripted protocol (Lecci A et al, Br J Pharmacol 130: 331-38, 2000).

Briefly, female Sprague Daley rats (˜200 g) are housed under normal conditions for laboratory rats in a 12:12-h light: dark cycle. Compound examples are dissolved in relevant vehicle (e.g. DMSO/Water) (5/95) (v/v) and are administrated by oral gavage in different concentrations e.g. one hour before application of cyclophosphamide (100 mg/kg) by i.p. injection. Additional 1.5 hours after cyclophosphamide administration each rat is transferred to metabolic cage and voiding frequency is recorded for the next 15 hours. Total amount of urine (ml) is collected via a plastic tube attached to the metabolic cages. The urine collecting tube is connected to a weight-sensitive sensor and a pressure transducer connected via an amplifier (ADIstruments) to the data acquisition software program (LabChart, ADInstruments) and a computer. The micturition per hour is recorded and the AUC during the plateau phase of the micturition (4-10 hours after transfer to metabolic cages) is calculated for each animal with e.g. GraphPad Prism 6 program.

### Isolated Bladder Stripes in the Organbath System (Rat)

The aim of this study was to test the efficacy of compound example 253 on prevention and attenuation of BDKRB1 agonist induced bladder stripe contractions, as a functional model for over active bladder. 5 l of Krebs-Ringer-solution have been freshly prepared by adding NaCl (34.5 g), NaHCO₃ (10.5 g), glucose (9.9 g), MgSO₄ (1.5 g) and KH₂PO₄ (0.8 g) to 4984.65 ml of water, stirring, and adding 8.5 ml of 10% KCL in water (m/v) and 6.85 ml of 20% CaCl₂ in water (m/v). The solution was pre-heated to 37° C. in the organ bath system (DMT, DMT750TOBS) before the start of the experiment.

After removing the bladder of a cyclophosphamide (150 mg/kg, i.p., sigma aldrich) pretreated rats (Sprague Dawley, female, Charles River Sulzfeld), bladder strips (˜2×8 mm) were cut from the bladder and mounted under 1-5 g of resting tension in glass organ baths, while the bath was continuously bubbled with carbogen gas (95% O₂ and 5% CO₂) and thermostated at 37° C. Contractile responses of the bladder stripes were measured using isometric tension transducers (DMT) and recorded using a data acquisition system (PowerLab and LabChart 8 software, ADInstruments).

After two hours of equilibration, tissues were treated with KCl 50 mM to check viability. 1 μM of carbachol was added to organ bath to elicit maximum contractile response and after five minutes of incubation it was washed out.

After 30 min of resting period a single concentration of 0.5 μM des-Arg⁹-Bradykinin, a BDKRB1 agonist (sigma aldrich), was added to the organ baths, incubated for 20 minutes and contractility has been measured and set 100%. After wash-out of the BDKRB1 agonist and additional 60 minutes of restring period different concentrations of compound example 253 (each concentration of compound example 253 was tested on a fresh bladder stripe, no repeated measurement per bladder stripe) have been added to the organ baths and incubated for 15 minutes. 0.5 μM des-Arg⁹-Bradykinin was added, incubated for 20 minutes and washed out. The effect of compound example 253 on inhibition of contraction of the bladder stripes induced by des-Arg⁹-Bradykinin has been measured and expressed as “% inhibition of des-Arg⁹-Bradykinin-induced plateau of contraction”. IC₅₀ value of compound example 253 has been calculated. Data analysis and generation of a dose-response-curve has been performed by GraphPad Prism 7 program.

### Results:

Preventive treatment of tested compound example 253 dose-dependently inhibited des-Arg⁹-Bradykinin (BDKRB1 agonist)-induced contractions of bladder stripes from previously cyclophosphamide (CYP) treated rats.

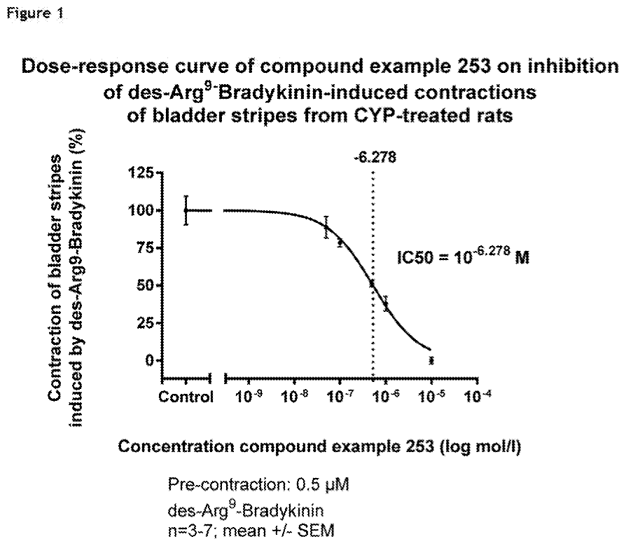

Preventive treatment using different concentrations of compound example 253 showed dose-dependent efficacy on inhibiting BDKRB1 agonist (0.5 μM des-Arg⁹-Bradykinin)-induced contractions of bladder stripes from previously cyclophosphamide (CYP) treated rats with an IC₅₀ of 10^{-6.278} M (FIG. 1). Data are shown as mean+/−SEM of 3-7 bladder stripes tested per concentration of compound example 253.

Diet-induced obese rat model

The diet-induced obese rat model is a relevant model to evaluate compounds targeting insulin sensitivity. In addition glucose-, fat-, muscle- and liver-dependent metabolic changes can be evaluated. Male Sprague-Dawley rats, at 8 weeks of age, are housed in small groups in ventilated and enriched housing cages. After acclimation phase, rats are randomized into groups (n=10-12, each). One group receives normal control chow (e.g. Research Diets, Inc., Research Diet ref #D12489B) during the whole experiment. Other groups receive normal control chow diet during the acclimation period, and then high fat/high sucrose diet (e.g. Research Diets, Inc., Research Diet ref #D12266B) until the end of the experiment.

After feeding the high fat/high sucrose diet for eight weeks, rats are fasted for 6-hours, and bled. Fasting blood glucose (mg/dL) and plasma insulin (μU/mL) are measured to calculate the Homeostatic Model Assessment (HOMA) of Insulin Resistance (IR) HOMA-IR index ([mM×μU/mL]/22.5).

Body weight is measured and rats are randomized to treatment groups according to their body weight and HOMA-IR index. Once daily p.o.-treatment of 5 ml/kg of compound examples dissolved in suitable vehicle (e.g. DMSO/Water) (5/95) (v/v)) starts in the high fat/high sucrose groups. Pioglitazone is chosen as positive control. Treatment in the groups is continued for e.g. 30 days as follows:

Group 1: normal chow, no treatment
Group 2: high fat/high sucrose diet, compound examples vehicle
Group 3: high fat/high sucrose diet, compound examples e.g. 15 mg/kg
Group 4: high fat/high sucrose diet, compound examples of higher dose, e.g. 60 mg/kg
Group 5: high fat/high sucrose diet, 0.5% methyl cellulose vehicle
Group 6: high fat/high sucrose diet, pioglitazone 10 mg/kg

At day 14 and day 26 of treatment, rats are weighed and 6-hour fasted and bled to measure fasting blood glucose and plasma insulin, plasma triglycerides, free fatty acids and total cholesterol. HOMA-IR index is calculated from blood glucose and plasma insulin values. In addition, at treatment day 26, glycated hemoglobin (HbA1C) and leptin are measured and rats undergo an insulin tolerance test with insulin (0.3 U/kg, 0.5 mL/kg) which is injected intraperitoneally. Blood glucose is measured at different timepoints 0 to 120 minutes after insulin injection.

After recovery, at day 28 of treatment, rats are weighed and 6-hour fasted and bled to measure fasting blood glucose and plasma insulin. Rats undergo an oral glucose tolerance test with glucose (2.5 g/kg) which is administered orally. Blood glucose is measured at different timepoints 0 to 150 minutes after glucose load. Plasma insulin levels are measured as well.

At day 30 of treatment, rats are sacrificed; plasma is withdrawn for pharmacokinetic evaluation of compound examples. Fat, muscle, pancreas and liver are taken and processed to allow gene expression analysis and histological evaluation. Liver specimens are histologically processed, e.g. hematoxylin-eosin staining, sirius red staining to evaluate fat and lipid deposition, inflammatory cell infiltration, degree of fibrotic changes, hepatocyte ballooning, apoptosis and necrosis.

1. A compound of formula (I): 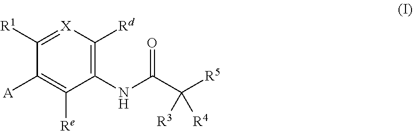
in which
A represents tetrazolyl which is attached to the rest of the molecule by the carbon atom;
R¹ represents
phenyl,
C₃-C₇-cycloalkyl,
5- to 7-membered lactam,
5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
bicyclic 8- to 10-membered heteroaryl containing 1, 2 or 3 heteroatoms or heteroatom-containing groups independently selected from NH, N, O, S, SO and SO₂,
wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, benzyl, NHR², N(R²)₂, NH(C₃-C₅-cycloalkyl), halogen, CN, NHSO₂R², SO₂R² or 4- to 7-membered heterocycloalkyl containing 1 or 2 heteroatoms or heteroatom-containing groups selected from NH, —NR², N, O, S, SO and SO₂, and
wherein independently, if R¹ represents 5- to 7-membered lactam, 5-membered heteroaryl or bicyclic 8- to 10-membered heteroaryl, each ring nitrogen atom, if present, of said R¹ is optionally substituted with a substituent R^{1b} wherein R^{1b} represents C₁-C₅-alkyl, C₃-C₅-cycloalkyl, —(C₁-C₃-alkyl)-(C₃-C₅-cycloalkyl), benzyl, SO₂R² or 4- to 7-membered heterocycloalkyl containing 1 or 2 heteroatoms or heteroatom-containing groups selected from NH, —NR², N, O, S, SO and SO₂, and
if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F, and
if R^{1a} and/or R^{1b} represent 4- to 7-membered heterocycloalkyl, each carbon atom of said 4- to 7-membered heterocycloalkyl is optionally substituted with one or more substituents independently selected from the group consisting of OH, OR² and F;
R² represents C₁-C₅-alkyl optionally substituted with 1 to 5 fluorine atoms;
X represents CR^{c} or N;
R^{c} represents H, halogen, CN, C₁-C₅-alkyl, or —OC₁-C₅-alkyl wherein said C₁-C₅-alkyl and —OC₁-C₅-alkyl are optionally substituted with 1 to 5 fluorine atoms;
R^{d} represents H, halogen, CN, OH, C₁-C₅-alkyl, or —OC₁-C₅-alkyl wherein said C₁-C₅-alkyl and —OC₁-C₅-alkyl are optionally substituted with 1 to 5 fluorine atoms;
R^{e} represents H, halogen or OH;
R³ and R⁴ are the same or different and represent C₁-C₃-alkyl, or R³ and R⁴ may be conjoined together with the carbon atom to which R³ and R⁴ are attached to form a C₃-C₅-cycloalkyl, which is optionally substituted with one or two C₁-C₃-alkyls wherein said C₁-C₃-alkyls are optionally substituted with 1 to 5 fluorine atoms;
R⁵ represents
C₁-C₅-alkyl,
(CH₂)ₚ-phenyl,
—(CH₂)ₚ-(C₅-C₇-cycloalkyl),
5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
bicyclic 8- to 10-membered heteroaryl containing 1, 2 or 3 heteroatoms or heteroatom-containing groups independently selected from NH, N, O, S, SO and SO₂,
wherein said R⁵ is optionally substituted at one or more carbon atoms with 1 to 3 substituents which are the same or different and selected from the group consisting of C₁-C₅-alkyl, —OC₁-C₅-alkyl, halogen, OH and CN, wherein said C₁-C₅-alkyl and —OC₁-C₅-alkyl independently are optionally substituted with substituent(s) selected from the group consisting of OH, OR², and 1 to 5 fluorine atoms, and
wherein independently, if R⁵ represents 5-membered heteroaryl or bicyclic 8- to 10-membered heteroaryl, each ring nitrogen atom, if present, of said R⁵ is optionally substituted with C₁-C₅-alkyl, which is optionally substituted with OH, OR² or 1 to 5 fluorine atoms; and
p is 0 or 1,
or an isomer, enantiomer, diastereomer, racemate, hydrate, solvate, or a salt thereof, or a mixture of the same.

2. The compound according to claim 1, wherein
R¹ represents
6-membered heteroaryl containing 1 or 2 N atoms, in particular pyridinyl, pyrimidinyl or pyrazinyl,
wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

3. The compound according to claim 1, wherein
R¹ represents
5-membered heteroaryl, wherein said 5-membered heteroaryl contains 1, 2 or 3 heteroatoms or heteroatom-containing groups independently selected from the group consisting of S, N, NH, and O, in particular pyrazolyl, imidazolyl or thiophenyl,
wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F.

4. The compound according to claim 1, wherein
R¹ represents phenyl, pyridinyl, pyrimidinyl, pyrazolyl, imidazolyl, thiophenyl, benzothiophenyl, benzothiazolyl, thienopyridinyl, indolyl or indazolyl,
wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
wherein independently, if R¹ represents pyrazolyl, imidazolyl, benzothiazolyl, thienopyridinyl, indolyl or indazolyl, each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR², and F.

5. The compound according to claim 1, wherein
X is CR^{c}; and
R^{c} represents H or F, optionally H.

6. The compound according to claim 1, wherein
R³ and R⁴ form together an unsubstituted cyclopropyl ring.

7. The compound according to claim 1, wherein
R⁵ represents (CH₂)ₚ-phenyl, wherein p is 0 or 1, optionally phenyl, optionally substituted at one or more carbon atoms with 1 or 2 substituents which are the same or different and selected from the group consisting of C₁-C₅-alkyl, —OC₁-C₅-alkyl, F and Cl, wherein independently said C₁-C₅-alkyl and —OC₁-C₅-alkyl are optionally substituted with substituent(s) selected from the group consisting of OH, OR², and 1 to 5 fluorine atoms.

8. The compound according to claim 1, wherein
R^{d} represents H, OH, F or methyl; optionally H.

9. The compound according to claim 1, wherein
R^{e} represents H or F; optionally H.

10. The compound according to claim 1, wherein
R¹ represents pyridinyl, optionally pyridin-3-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN; and
wherein one of said R^{1a} is optionally positioned para to the carbon atom which links the pyridinyl, in particular pyridin-3-yl, to the rest of the molecule; and
wherein independently said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl are optionally substituted with one or more substituents independently selected from the group consisting of OH, OR² and F;
R³ and R⁴ form together an unsubstituted cyclopropyl ring;
X represents CR^{c} or N, optionally CR^{c};
R^{c} represents H, F, Cl, methyl, trifluoromethyl or CN, optionally H or F;
R^{d} represents H or F, optionally H; and
R^{e} represents H or F, optionally H.

11. The compound according to claim 1, wherein
R³ and R⁴ form together an unsubstituted cyclopropyl ring;
X represents CR^{c} or N, optionally CR^{c};
R^{c} represents H, F, Cl, methyl, trifluoromethyl or CN, optionally H or F;
R^{d} represents H or F, optionally H;
R^{e} represents H or F, optionally H; and
R⁵ represents phenyl optionally substituted at one or more carbon atoms with 1 or 2 substituents which are the same or different and selected from the group consisting of methyl, trifluoromethyl, trifluoromethoxy, F and Cl,
wherein if the substituent or at least one of said substituents is F, it is optionally positioned ortho to the carbon atom which links the phenyl to the rest of the molecule, and
wherein if the substituent or at least one of said substituents is methyl, trifluoromethyl, trifluoromethoxy or Cl, said substituent is optionally positioned para to the carbon atom which links the phenyl to the rest of the molecule.

12. The compound according to claim 1, wherein
R¹ represents pyridinyl, optionally pyridin-3-yl,
wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN; and
wherein one of said R^{1a} is optionally positioned para to the carbon atom which links the pyridinyl optionally pyridin-3-yl, to the rest of the molecule; and
wherein independently said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl are optionally substituted with one or more substituents independently selected from the group consisting of OH, OR² and F;
R³ and R⁴ form together an unsubstituted cyclopropyl ring;
R^{d} represents H or F, optionally H;
R^{e} represents H or F, optionally H;
X represents CR^{c} or N, optionally CR^{c};
R^{c} represents H, F, Cl or methyl, optionally H or F; and
R⁵ represents phenyl substituted at one or more carbon atoms with 1 or 2 substituents which are the same or different and selected from the group consisting of C₁-C₅-alkyl, OC₁-C₅-alkyl, F and Cl,
wherein the substituent or at least one of said substituents is C₁-C₅-alkyl, OC₁-C₅-alkyl or Cl, said substitutent or said at least one of said substitutents is optionally positioned para to the carbon atom which links the phenyl to the rest of the molecule; and
wherein independently said C₁-C₅-alkyl and OC₁-C₅-alkyl are optionally substituted with 1 to 5 fluorine atoms.

13. The compound according to claim 1, wherein
R¹ represents 5-membered heteroaryl, wherein said 5-membered heteroaryl contains 1, 2 or 3 heteroatoms or heteroatom-containing groups independently selected from the group consisting of S, N, NH, and O, optionally pyrazolyl, imidazolyl or thiophenyl,
wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, halogen or CN, and
wherein independently each ring nitrogen atom, if present, of said R¹ is optionally substituted with 1 substituent R^{1b} wherein R^{1b} represents C₁-C₅-alkyl, —(C₁-C₃-alkyl)-(C₃-C₅-cycloalkyl) or C₃-C₅-cycloalkyl, and
if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl and/or if R^{1b} represents C₁-C₅-alkyl, C₃-C₅-cycloalkyl or —(C₁-C₃-alkyl)-(C₃-C₅-cycloalkyl), said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR² and F,
R³ and R⁴ form together an unsubstituted cyclopropyl ring;
R^{d} represents H or F, optionally H;
R^{e} represents H or F, optionally H;
X represents CR^{c} or N, optionally CR^{c};
R^{c} represents H, F, Cl or methyl, optionally H or F; and
R⁵ represents phenyl substituted at one or more carbon atoms with 1 or 2 substituents which are the same or different and selected from the group consisting of C₁-C₅-alkyl, OC₁-C₅-alkyl, F and Cl,
wherein the substituent or at least one of said substituents is C₁-C₅-alkyl, OC₁-C₅-alkyl or Cl, said substitutent or said at least one of said substitutents is optionally positioned para to the carbon atom which links the phenyl to the rest of the molecule; and
wherein independently said C₁-C₅-alkyl and OC₁-C₅-alkyl are optionally substituted with 1 to 5 fluorine atoms.

14. The compound according to claim 1, namely
1-(3-chlorophenyl)-N-[3′,4′-dimethoxy-2-(1H-tetrazol-5-yl)biphenyl-4-yl]cyclopropanecarboxamide;
N-[3′,4′-dimethoxy-2-(1H-tetrazol-5-yl)biphenyl-4-yl]-1-(3-fluoro-4-methylphenyl)cyclopropanecarboxamide;
N-[3′,4′-dimethoxy-2-(1H-tetrazol-5-yl)biphenyl-4-yl]-1-[3-(trifluoromethyl)phenyl]cyclopropanecarboxamide;
1-(4-chlorophenyl)-N-[3′,4′-dimethoxy-2-(1H-tetrazol-5-yl)biphenyl-4-yl]cyclopropanecarboxamide;
N-[3′,4′-dimethoxy-2-(1H-tetrazol-5-yl)biphenyl-4-yl]-1-[4-(trifluoromethyl)phenyl]cyclopropanecarboxamide;
N-[3′,4′-dimethoxy-2-(1H-tetrazol-5-yl)biphenyl-4-yl]-1-(4,4-dimethylcyclohexyl)cyclopropanecarboxamide;
N-[4-(6-ethoxypyridin-3-yl)-3-(1H-tetrazol-5-yl)phenyl]-1-(4-ethylphenyl)cyclopropanecarboxamide;
1-(4-chloro-3-fluorophenyl)-N-[4-(6-ethoxypyridin-3-yl)-3-(1H-tetrazol-5-yl)phenyl]cyclopropanecarboxamide;
1-(3-chloro-4-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[2-fluoro-4-(trifluoromethyl)phenyl]cyclopropanecarboxamide;
1-(3-chloro-2-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[4-(trifluoromethoxy)phenyl]cyclopropanecarboxamide;
1-(4-chloro-2-fluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-(3-fluoro-4-methylphenyl)cyclopropanecarboxamide;
1-(3-chloro-5-fluorophenyl)-N-[4-(6-ethoxypyridin-3-yl)-3-(1H-tetrazol-5-yl)phenyl]cyclopropanecarboxamide;
1-(3,5-difluorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-(3-ethylphenyl)cyclopropanecarboxamide;
N-[4-(6-ethoxypyridin-3-yl)-3-(1H-tetrazol-5-yl)phenyl]-1-(3-propylphenyl)cyclopropanecarboxamide;
N-[4-(6-ethoxypyridin-3-yl)-3-(1H-tetrazol-5-yl)phenyl]-1-[3-(trifluoromethyl)phenyl]cyclopropanecarboxamide;
1-(4-chlorophenyl)-N-[4-(6-ethoxypyridin-3-yl)-3-(1H-tetrazol-5-yl)phenyl]cyclopropanecarboxamide;
N-[4-(6-ethoxypyridin-3-yl)-3-(1H-tetrazol-5-yl)phenyl]-1-[4-(trifluoromethyl)phenyl]cyclopropanecarboxamide;
1-(4,4-dimethylcyclohexyl)-N-[4-(6-ethoxypyridin-3-yl)-3-(1H-tetrazol-5-yl)phenyl]cyclopropanecarboxamide;
1-(3-chlorophenyl)-N-[4-(6-propoxypyridin-3-yl)-3-(1H-tetrazol-5-yl)phenyl]cyclopropanecarboxamide;
N-[4-(6-propoxypyridin-3-yl)-3-(1H-tetrazol-5-yl)phenyl]-1-[4-(trifluoromethyl)phenyl]cyclopropanecarboxamide;
1-(2,4-difluorophenyl)-N-[4-(6-propoxypyridin-3-yl)-3-(1H-tetrazol-5-yl)phenyl]cyclopropanecarboxamide;
1-(3-chlorophenyl)-N-[4′-ethoxy-3′-methoxy-2-(1H-tetrazol-5-yl)biphenyl-4-yl]cyclopropanecarboxamide;
1-(3-chlorophenyl)-N-{4-[6-(methoxymethyl)pyridin-3-yl]-3-(1H-tetrazol-5-yl)phenyl}cyclopropanecarboxamide;
1-(3-chlorophenyl)-N-[2-(1H-tetrazol-5-yl)-4′-(trifluoromethyl)biphenyl-4-yl]cyclopropanecarboxamide;
1-(4-chloro-3-fluorophenyl)-N-{3-(1H-tetrazol-5-yl)-4-[6-(trifluoromethyl)pyridin-3-yl]phenyl}cyclopropanecarboxamide;
1-(3-chlorophenyl)-N-{4-[6-(propan-2-yloxy)pyridin-3-yl]-3-(1H-tetrazol-5-yl)phenyl}cyclopropanecarboxamide;
N-{4-[6-(propan-2-yloxy)pyridin-3-yl]-3-(1H-tetrazol-5-yl)phenyl}-1-[4-(trifluoromethyl)phenyl]cyclopropanecarboxamide;
1-(3-chlorophenyl)-N-[4-(5-methoxypyridin-3-yl)-3-(1H-tetrazol-5-yl)phenyl]cyclopropanecarboxamide;
1-(3-chlorophenyl)-N-[4-(6-ethoxy-5-methylpyridin-3-yl)-3-(1H-tetrazol-5-yl)phenyl]cyclopropanecarboxamide;
1-(3-chlorophenyl)-N-[4-(6-ethoxy-5-fluoropyridin-3-yl)-3-(1H-tetrazol-5-yl)phenyl]cyclopropanecarboxamide;
N-[4-(6-ethoxy-5-fluoropyridin-3-yl)-3-(1H-tetrazol-5-yl)phenyl]-1-[4-(trifluoromethyl)phenyl]cyclopropanecarboxamide;
1-(3-chlorophenyl)-N-[4-(2-ethylpyridin-4-yl)-3-(1H-tetrazol-5-yl)phenyl]cyclopropanecarboxamide;
1-[2-fluoro-4-(trifluoromethyl)phenyl]-N-{3-(1H-tetrazol-5-yl)-4-[6-(trifluoromethyl)pyridin-3-yl]phenyl}cyclopropanecarboxamide;
1-(3-chlorophenyl)-N-[4-(6-methylpyridin-3-yl)-3-(1H-tetrazol-5-yl)phenyl]cyclopropanecarboxamide;
1-[2-fluoro-4-(trifluoromethyl)phenyl]-N-[4-(6-methylpyridin-3-yl)-3-(1H-tetrazol-5-yl)phenyl]cyclopropanecarboxamide;
1-(3-chloro-2-fluorophenyl)-N-[4-(2-methylpyridin-4-yl)-3-(1H-tetrazol-5-yl)phenyl]cyclopropanecarboxamide;
1-[2-fluoro-4-(trifluoromethyl)phenyl]-N-[4-(2-methylpyridin-4-yl)-3-(1H-tetrazol-5-yl)phenyl]cyclopropanecarboxamide;
1-(3-chlorophenyl)-N-[4-(1-methyl-1H-indazol-6-yl)-3-(1H-tetrazol-5-yl)phenyl]cyclopropanecarboxamide;
1-(3-chloro-2-fluorophenyl)-N-[4-(1-methyl-1H-indazol-6-yl)-3-(1H-tetrazol-5-yl)phenyl]cyclopropanecarboxamide;
1-[2-fluoro-4-(trifluoromethyl)phenyl]-N-[4-(1-methyl-1H-indazol-6-yl)-3-(1H-tetrazol-5-yl)phenyl]cyclopropanecarboxamide;
N-[2-fluoro-3′,4′-dimethoxy-6-(1H-tetrazol-5-yl)biphenyl-4-yl]-1-[4-(trifluoromethyl)phenyl]cyclopropanecarboxamide;
1-(3-chlorophenyl)-N-[3′-methoxy-2-(1H-tetrazol-5-yl)-4′-(trifluoromethyl)biphenyl-4-yl]cyclopropanecarboxamide;
1-(3-chlorophenyl)-N-[2-fluoro-3′,4′-dimethoxy-6-(1H-tetrazol-5-yl)biphenyl-4-yl]cyclopropanecarboxamide;
1-(3-chlorophenyl)-N-[4-(6-ethoxypyridin-3-yl)-3-fluoro-5-(1H-tetrazol-5-yl)phenyl]cyclopropanecarboxamide;
N-[4-(6-ethoxypyridin-3-yl)-3-fluoro-5-(1H-tetrazol-5-yl)phenyl]-1-[2-fluoro-4-(trifluoromethyl)phenyl]cyclopropanecarboxamide;
N-{3-fluoro-5-(1H-tetrazol-5-yl)-4-[6-(trifluoromethyl)pyridin-3-yl]phenyl}-1-[2-fluoro-4-(trifluoromethyl)phenyl]cyclopropanecarboxamide;
1-(3-chlorophenyl)-N-[3-fluoro-4-(6-methylpyridin-3-yl)-5-(1H-tetrazol-5-yl)phenyl]cyclopropanecarboxamide;
1-(3-chlorophenyl)-N-[3-fluoro-4-(2-methylpyridin-4-yl)-5-(1H-tetrazol-5-yl)phenyl]cyclopropanecarboxamide;
N-[3-chloro-4-(6-ethoxypyridin-3-yl)-5-(1H-tetrazol-5-yl)phenyl]-1-(3-chlorophenyl)cyclopropanecarboxamide;
N-[3-chloro-4-(6-ethoxypyridin-3-yl)-5-(1H-tetrazol-5-yl)phenyl]-1-(3-chloro-2-fluorophenyl)cyclopropanecarboxamide;
1-(3-chlorophenyl)-N-[4-(6-ethoxypyridin-3-yl)-3-(1H-tetrazol-5-yl)phenyl]cyclopropanecarboxamide;
N-{4-[6-(difluoromethyl)pyridin-3-yl]-3-(1H-tetrazol-5-yl)phenyl}-1-[2-fluoro-4-(trifluoromethyl)phenyl]cyclopropanecarboxamide;
1-(3-chlorophenyl)-N-{4-[6-(difluoromethyl)pyridin-3-yl]-3-(1H-tetrazol-5-yl)phenyl}cyclopropanecarboxamide;
1-(3-chlorophenyl)-N-[4-(6-ethoxypyridin-3-yl)-2-hydroxy-5-(1H-tetrazol-5-yl)phenyl]cyclopropanecarboxamide;
N-[3-fluoro-4-(2-methylpyridin-4-yl)-5-(1H-tetrazol-5-yl)phenyl]-1-[2-fluoro-4-(trifluoromethyl)phenyl]cyclopropanecarboxamide;
N-[3-fluoro-4-(6-methylpyridin-3-yl)-5-(1H-tetrazol-5-yl)phenyl]-1-[2-fluoro-4-(trifluoromethyl)phenyl]cyclopropanecarboxamide;
N-{3-fluoro-5-(1H-tetrazol-5-yl)-4-[2-(trifluoromethyl)pyridin-4-yl]phenyl}-1-[2-fluoro-4-(trifluoromethyl)phenyl]cyclopropanecarboxamide;
1-(4-chlorophenyl)-N-{4-[6-(difluoromethyl)pyridin-3-yl]-3-(1H-tetrazol-5-yl)phenyl}cyclopropanecarboxamide;
N-{4-[6-(difluoromethyl)pyridin-3-yl]-3-(1H-tetrazol-5-yl)phenyl}-1-[4-(trifluoromethyl)phenyl]cyclopropanecarboxamide;
N-{4-[6-(difluoromethyl)pyridin-3-yl]-3-(1H-tetrazol-5-yl)phenyl}-1-[4-(trifluoromethoxy)phenyl]cyclopropanecarboxamide;
1-(4-chloro-3-fluorophenyl)-N-{4-[6-(difluoromethyl)pyridin-3-yl]-3-(1H-tetrazol-5-yl)phenyl}cyclopropanecarboxamide;
1-(4-chloro-2-fluorophenyl)-N-{4-[6-(difluoromethyl)pyridin-3-yl]-3-(1H-tetrazol-5-yl)phenyl}cyclopropanecarboxamide;
N-{4-[6-(difluoromethyl)pyridin-3-yl]-3-(1H-tetrazol-5-yl)phenyl}-1-(2-fluoro-4-methylphenyl)cyclopropanecarboxamide;
N-{4-[6-(difluoromethyl)pyridin-3-yl]-3-(1H-tetrazol-5-yl)phenyl}-1-[2-fluoro-4-(trifluoromethoxy)phenyl]cyclopropanecarboxamide;
N-{4-[6-(1,1-difluoroethyl)pyridin-3-yl]-3-(1H-tetrazol-5-yl)phenyl}-1-[4-(trifluoromethyl)phenyl]cyclopropanecarboxamide;
N-{4-[6-(1,1-difluoroethyl)pyridin-3-yl]-3-(1H-tetrazol-5-yl)phenyl}-1-[4-(trifluoromethoxy)phenyl]cyclopropanecarboxamide;
N-{4-[6-(1,1-difluoroethyl)pyridin-3-yl]-3-(1H-tetrazol-5-yl)phenyl}-1-(2-fluoro-4-methylphenyl)cyclopropanecarboxamide;
N-{4-[6-(1,1-difluoroethyl)pyridin-3-yl]-3-(1H-tetrazol-5-yl)phenyl}-1-[2-fluoro-4-(trifluoromethyl)phenyl]cyclopropanecarboxamide;
N-{4-[6-(1,1-difluoroethyl)pyridin-3-yl]-3-(1H-tetrazol-5-yl)phenyl}-1-[2-fluoro-4-(trifluoromethoxy)phenyl]cyclopropanecarboxamide;
1-(3-chlorophenyl)-N-{4-[6-(1,1-difluoropropyl)pyridin-3-yl]-3-(1H-tetrazol-5-yl)phenyl}cyclopropanecarboxamide;
1-(3,4-difluorophenyl)-N-{4-[6-(1,1-difluoropropyl)pyridin-3-yl]-3-(1H-tetrazol-5-yl)phenyl}cyclopropanecarboxamide;
N-{4-[6-(1,1-difluoropropyl)pyridin-3-yl]-3-(1H-tetrazol-5-yl)phenyl}-1-(2-fluoro-4-methylphenyl)cyclopropanecarboxamide;
N-{4-[6-(1,1-difluoropropyl)pyridin-3-yl]-3-(1H-tetrazol-5-yl)phenyl}-1-[2-fluoro-4-(trifluoromethyl)phenyl]cyclopropanecarboxamide;
N-{4-[6-(1,1-difluoropropyl)pyridin-3-yl]-3-(1H-tetrazol-5-yl)phenyl}-1-[2-fluoro-4-(trifluoromethoxy)phenyl]cyclopropanecarboxamide;
1-[3-fluoro-4-(trifluoromethyl)phenyl]-N-{3-(1H-tetrazol-5-yl)-4-[6-(trifluoromethyl)pyridin-3-yl]phenyl}cyclopropanecarboxamide;
1-[2-chloro-4-(trifluoromethyl)phenyl]-N-{3-(1H-tetrazol-5-yl)-4-[6-(trifluoromethyl)pyridin-3-yl]phenyl}cyclopropanecarboxamide;
1-[2-fluoro-4-(trifluoromethoxy)phenyl]-N-{3-(1H-tetrazol-5-yl)-4-[6-(trifluoromethyl)pyridin-3-yl]phenyl}cyclopropanecarboxamide;
N-{3-chloro-5-(1H-tetrazol-5-yl)-4-[6-(trifluoromethyl)pyridin-3-yl]phenyl}-1-[2-fluoro-4-(trifluoromethyl)phenyl]cyclopropanecarboxamide;
1-(3-chlorophenyl)-N-[4-(5-methylpyridin-3-yl)-3-(1H-tetrazol-5-yl)phenyl]cyclopropanecarboxamide;
N-[4-(5-chloro-6-ethoxypyridin-3-yl)-3-(1H-tetrazol-5-yl)phenyl]-1-(3-chlorophenyl)cyclopropanecarboxamide;
1-(3-chlorophenyl)-N-[4-(6-ethylpyridin-3-yl)-3-(1H-tetrazol-5-yl)phenyl]cyclopropanecarboxamide;
1-[2-fluoro-4-(trifluoromethoxy)phenyl]-N-{3-(1H-tetrazol-5-yl)-4-[4-(trifluoromethyl)-1H-pyrazol-1-yl]phenyl}cyclopropanecarboxamide;
N-[4-(4-ethyl-1H-pyrazol-1-yl)-3-(1H-tetrazol-5-yl)phenyl]-1-[2-fluoro-4-(trifluoromethyl)phenyl]cyclopropanecarboxamide;
N-[4-(4-ethyl-1H-pyrazol-1-yl)-3-(1H-tetrazol-5-yl)phenyl]-1-[2-fluoro-4-(trifluoromethoxy)phenyl]cyclopropanecarboxamide;
N-[4-(1-benzothiophen-2-yl)-3-(1H-tetrazol-5-yl)phenyl]-1-[2-fluoro-4-(trifluoromethyl)phenyl]cyclopropanecarboxamide;
1-[2-fluoro-4-(trifluoromethyl)phenyl]-N-{3-(1H-tetrazol-5-yl)-4-[3-(2,2,2-trifluoroethyl)-1H-pyrazol-1-yl]phenyl}cyclopropanecarboxamide;
1-[2-fluoro-4-(trifluoromethoxy)phenyl]-N-{3-(1H-tetrazol-5-yl)-4-[3-(2,2,2-trifluoroethyl)-1H-pyrazol-1-yl]phenyl}cyclopropanecarboxamide;
N-[4-(4-chloro-1H-pyrazol-1-yl)-3-(1H-tetrazol-5-yl)phenyl]-1-[2-fluoro-4-(trifluoromethoxy)phenyl]cyclopropanecarboxamide;
1-[2-fluoro-4-(trifluoromethyl)phenyl]-N-{4-[4-(propan-2-yl)-1H-pyrazol-1-yl]-3-(1H-tetrazol-5-yl)phenyl}cyclopropanecarboxamide;
N-[4-(4-tert-butyl-1H-imidazol-1-yl)-3-(1H-tetrazol-5-yl)phenyl]-1-[2-fluoro-4-(trifluoromethoxy)phenyl]cyclopropanecarboxamide;
N-[4-(4-tert-butyl-1H-pyrazol-1-yl)-3-(1H-tetrazol-5-yl)phenyl]-1-[2-fluoro-4-(trifluoromethyl)phenyl]cyclopropanecarboxamide;
N-[4-(4-tert-butyl-1H-pyrazol-1-yl)-3-(1H-tetrazol-5-yl)phenyl]-1-[2-fluoro-4-(trifluoromethoxy)phenyl]cyclopropanecarboxamide; or
1-[2-fluoro-4-(trifluoromethoxy)phenyl]-N-{4-[4-(propan-2-yl)-1H-pyrazol-1-yl]-3-(1H-tetrazol-5-yl)phenyl}cyclopropanecarboxamide.

15. A pharmaceutical composition comprising a compound according to claim 1, or an isomer, enantiomer, diastereomer, racemate, hydrate, solvate, or salt thereof, optionally a pharmaceutically acceptable salt thereof, or a mixture of same, and a pharmaceutically acceptable diluent or carrier.

16. A product comprising a compound according to claim 1, or an isomer, enantiomer, diastereomer, racemate, hydrate, solvate, or salt thereof, optionally a pharmaceutically acceptable salt thereof, or a mixture of same, or a pharmaceutical composition thereof, for prophylaxis or treatment of a disease or one or more disease syndromes, conditions, or symptoms.

17. A product comprising a compound according to claim 1, or an isomer, enantiomer, diastereomer, racemate, hydrate, solvate, or salt thereof, optionally a pharmaceutically acceptable salt thereof, or a mixture of same, or a pharmaceutical composition thereof, for preparation of a medicament for prophylaxis or treatment of a disease or one or more disease syndromes, conditions, or symptoms.

18. The product according to claim 16, wherein said disease or one or more disease syndromes, conditions, or symptoms are mediated by the Bradykinin B1 receptor and associated with pain and/or inflammation.

19. The product according to claim 16, wherein said disease or one or more disease syndromes, conditions, or symptoms are associated with pain selected from the group consisting of
visceral pain optionally related to pancreatitis, interstitial cystitis, renal colic, or prostatitis, chronic pelvic pain, or pain related to infiltrating endometriosis;
neuropathic pain optionally post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes optionally caused by optionally any lesion at any level of the nervous system optionally including pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis).

20. The product according to claim 16, wherein said disease or one or more disease syndromes, conditions, or symptoms are selected from or related to any one of the group consisting of
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including primary peritonitis, secondary peritonitis, septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

21. The product according to claim 20 wherein said disease or disease syndromes, conditions, or symptoms are selected from or related to endometriosis or endometriosis-associated pain, optionally to dysmenorrhea, dyspareunia, dysuria, or dyschezia.

22. The product according to claim 20 wherein said disease or disease syndromes, conditions, or symptoms are selected from correlated to overactive bladder, fibrosis of lung, kidney, heart and/or liver, diabetes type 1 and/or type 2, metabolic syndrome, gout, rheumatoid arthritis, and osteoarthritis including the related symptoms.

23. A compound according to claim 1, or an isomer, enantiomer, diastereomer, racemate, hydrate, solvate, or salt thereof, optionally a pharmaceutically acceptable salt thereof, or a mixture of same, or a pharmaceutical composition thereof, for manufacture of a medicament.

24. A compound according to claim 1, or an isomer, enantiomer, diastereomer, racemate, hydrate, solvate, or salt thereof, optionally a pharmaceutically acceptable salt thereof, or a mixture of same, or a pharmaceutical composition thereof, for use in treatment of disease mediated by the Bradykinin B1 receptor and associated with pain and inflammation.

25. A compound according to claim 1, or an isomer, enantiomer, diastereomer, racemate, hydrate, solvate, or salt thereof, optionally a pharmaceutically acceptable salt thereof, or a mixture of same, or a pharmaceutical composition thereof, for use in treatment of one or more pain syndromes including
visceral pain optionally related to pancreatitis, interstitial cystitis, renal colic, or prostatitis, chronic pelvic pain, or pain related to infiltrating endometriosis;
neuropathic pain optionally post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes optionally caused by optionally any lesion at any level of the nervous system optionally including pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis).

26. A compound according to claim 1 or an isomer, enantiomer, diastereomer, racemate, hydrate, solvate, or salt thereof, optionally a pharmaceutically acceptable salt thereof, or a mixture of same, or a pharmaceutical composition thereof, for use in treatment of a disease or one or more disease syndromes, conditions, or symptoms which are selected from or related to any one of the group consisting of
gynecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including primary peritonitis, secondary peritonitis, septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.
